SEQUENCE LISTING (243 sequences)

SEQ ID NO: 1-2 - skipped (2 entries)

SEQ ID NO: 3 - RNA, 10 nt, synthetic construct
  misc_feature 1..10: note = Description of Artificial Sequence: Synthetic oligonucleotide
  source 1..10: mol_type = other RNA
tttttttttt

SEQ ID NO: 4 - DNA, 10 bp, synthetic construct
  misc_feature 1..10: note = Description of Artificial Sequence: Synthetic oligonucleotide
  misc_feature 1..10: note = Description of Combined DNA/RNA Molecule: Synthetic oligonucleotide
  source 1..10: mol_type = other DNA
  misc_feature 5..6: note = RNA
  misc_feature 1..4: note = DNA
  misc_feature 7..10: note = DNA
tttttttttt

SEQ ID NO: 5-11 - skipped (7 entries)

SEQ ID NO: 12 - DNA, 10 bp, synthetic construct
  misc_feature 1..10: note = Description of Artificial Sequence: Synthetic oligonucleotide
  source 1..10: mol_type = other DNA
tttttttttt

SEQ ID NO: 13 - DNA, 12 bp, synthetic construct
  misc_feature 1..12: note = Description of Artificial Sequence: Synthetic oligonucleotide
  source 1..12: mol_type = other DNA
tttttttttttt

SEQ ID NO: 14 - DNA, 14 bp, synthetic construct
  misc_feature 1..14: note = Description of Artificial Sequence: Synthetic oligonucleotide
  source 1..14: mol_type = other DNA
tttttttttttttt

SEQ ID NO: 15-17 - skipped (3 entries)

SEQ ID NO: 18 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 19 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 20 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 21 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 22 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 23 - protein, 1285 aa, unidentified
  REGION 1..1285: note = Description of Unknown: Herbinix hemicellulosilytica sequence
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 24 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 25 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 26 - protein, 1224 aa, Bergeyella zoohelcum
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 27 - protein, 1126 aa, Prevotella intermedia
MEDDKKTTDSIRYELKDKHFWAAFLNLARHNVYITVNHINKILEEGEINRDGYETTLKNTWNEIKDINKKDRLSKLIIKHFPFLEAATYRLNPTDTTKQKEEKQAEAQSLESLRKSFFVFIYKLRDLRNHYSHYKHSKSLERPKFEEGLLEKMYNIFNASIRLVKEDYQYNKDINPDEDFKHLDRTEEEFNYYFTKDNEGNITESGLLFFVSLFLEKKDAIWMQQKLRGFKDNRENKKKMTNEVFCRSRMLLPKLRLQSTQTQDWILLDMLNELIRCPKSLYERLREEDREKFRVPIEIADEDYDAEQEPFKNTLVRHQDRFPYFALRYFDYNEIFTNLRFQIDLGTYHFSIYKKQIGDYKESHHLTHKLYGFERIQEFTKQNRPDEWRKFVKTFNSFETSKEPYIPETTPHYHLENQKIGIRFRNDNDKIWPSLKTNSEKNEKSKYKLDKSFQAEAFLSVHELLPMMFYYLLLKTENTDNDNEIETKKKENKNDKQEKHKIEEIIENKITEIYALYDTFANGEIKSIDELEEYCKGKDIEIGHLPKQMIAILKDEHKVMATEAERKQEEMLVDVQKSLESLDNQINEEIENVERKNSSLKSGKIASWLVNDMMRFQPVQKDNEGKPLNNSKANSTEYQLLQRTLAFFGSEHERLAPYFKQTKLIESSNPHPFLKDTEWEKCNNILSFYRSYLEAKKNFLESLKPEDWEKNQYFLKLKEPKTKPKTLVQGWKNGFNLPRGIFTEPIRKWFMKHRENITVAELKRVGLVAKVIPLFFSEEYKDSVQPFYNYHFNVGNINKPDEKNFLNCEERRELLRKKKDEFKKMTDKEKEENPSYLEFKSWNKFERELRLVRNQDIVTWLLCMELFNKKKIKELNVEKIYLKNINTNTTKKEKNTEEKNGEEKNIKEKNNILNRIMPMRLPIKVYGRENFSKNKKKKIRRNTFFTVYIEEKGTKLLKQGNFKALERDRRLGGLFSFVKTPSKAESKSNTISKLRVEYELGEYQKARIEIIKDMLALEKTLIDKYNSLDTDNFNKMLTDWLELKGEPDKASFQNDVDLLIAVRNAFSHNQYPMRNRIAFANINPFSLSSANTSEEKGLGIANQLKDKTHKTIEKIIEIEKPIETKE

SEQ ID NO: 28 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 29 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFAVFFKPDDFVLAKNRKEQLISVADGKECLTVSGFAFFICLFLDREQASGMLSRIRGFKRTDENWARAVHETFCDLCIRHPHDRLESSNTKEALLLDMLNELNRCPRILYDMLPEEERAQFLPALDENSMNNLSENSLDEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMDQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELRLLDPSSGHPFLSATMETAHRYTEGFYKCYLEKKREWLAKIFYRPEQDENTKRRISVFFVPDGEARKLLPLLIRRRMKEQNDLQDWIRNKQAHPIDLPSHLFDSKVMELLKVKDGKKKWNEAFKDWWSTKYPDGMQPFYGLRRELNIHGKSVSYIPSDGKKFADCYTHLMEKTVRDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 30 - protein, 1115 aa, Bacteroides pyogenes
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETAECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTSGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPLPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 31 - protein, 668 aa, Fusobacterium necrophorum
MTEKKSIIFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKIAFGFLFCEFDGNISKFKDEFDASNQEKIIQYHKNGEKYLTYFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKIKKFL

SEQ ID NO: 32 - protein, 796 aa, Tissierella sp.
MEKDKKGEKIDISQEMIEEDLRKILILFSRLRHSMVHYDYEFYQALYSGKDFVISDKNNLENRMISQLLDLNIFKELSKVKLIKDKAISNYLDKNTTIHVLGQDIKAIRLLDIYRDICGSKNGFNKFINTMITISGEEDREYKEKVIEHFNKKMENLSTYLEKLEKQDNAKRNNKRVYNLLKQKLIEQQKLKEWFGGPYVYDIHSSKRYKELYIERKKLVDRHSKLFEEGLDEKNKKELTKINDELSKLNSEMKEMTKLNSKYRLQYKLQLAFGFILEEFDLNIDTFINNFDKDKDLIISNFMKKRDIYLNRVLDRGDNRLKNIIKEYKFRDTEDIFCNDRDNNLVKLYILMYILLPVEIRGDFLGFVKKNYYDMKHVDFIDKKDKEDKDTFFHDLRLFEKNIRKLEITDYSLSSGFLSKEHKVDIEKKINDFINRNGAMKLPEDITIEEFNKSLILPIMKNYQINFKLLNDIEISALFKIAKDRSITFKQAIDEIKNEDIKKNSKKNDKNNHKDKNINFTQLMKRALHEKIPYKAGMYQIRNNISHIDMEQLYIDPLNSYMNSNKNNITISEQIEKIIDVCVTGGVTGKELNNNIINDYYMKKEKLVFNLKLRKQNDIVSIESQEKNKREEFVFKKYGLDYKDGEINIIEVIQKVNSLQEELRNIKETSKEKLKNKETLFRDISLINGTIRKNINFKIKEMVLDIVRMDEIRHINIHIYYKGENYTRSNIIKFKYAIDGENKKYYLKQHEINDINLELKDKFVTLICNMDKHPNKNKQTINLESNYIQNVKFIIP

SEQ ID NO: 33 - protein, 897 aa, Fusobacterium ulcerans
MENKGNNKKIDFDENYNILVAQIKEYFTKEIENYNNRIDNIIDKKELLKYSEKKEESEKNKKLEELNKLKSQKLKILTDEEIKADVIKIIKIFSDLRHSLMHYEYKYFENLFENKKNEELAELLNLNLFKNLTLLRQMKIENKTNYLEGREEFNIIGKNIKAKEVLGHYNLLAEQKNGFNNFINSFFVQDGTENLEFKKLIDEHFVNAKKRLERNIKKSKKLEKELEKMEQHYQRLNCAYVWDIHTSTTYKKLYNKRKSLIEEYNKQINEIKDKEVITAINVELLRIKKEMEEITKSNSLFRLKYKMQIAYAFLEIEFGGNIAKFKDEFDCSKMEEVQKYLKKGVKYLKYYKDKEAQKNYEFPFEEIFENKDTHNEEWLENTSENNLFKFYILTYLLLPMEFKGDFLGVVKKHYYDIKNVDFTDESEKELSQVQLDKMIGDSFFHKIRLFEKNTKRYEIIKYSILTSDEIKRYFRLLELDVPYFEYEKGTDEIGIFNKNIILTIFKYYQIIFRLYNDLEIHGLFNISSDLDKILRDLKSYGNKNINFREFLYVIKQNNNSSTEEEYRKIWENLEAKYLRLHLLTPEKEEIKTKTKEELEKLNEISNLRNGICHLNYKEIIEEILKTEISEKNKEATLNEKIRKVINFIKENELDKVELGFNFINDFFMKKEQFMFGQIKQVKEGNSDSITTERERKEKNNKKLKETYELNCDNLSEFYETSNNLRERANSSSLLEDSAFLKKIGLYKVKNNKVNSKVKDEEKRIENIKRKLLKDSSDIMGMYKAEVVKKLKEKLILIFKHDEEKRIYVTVYDTSKAVPENISKEILVKRNNSKEEYFFEDNNKKYVTEYYTLEITETNELKVIPAKKLEGKEFKTEKNKENKLMLNNHYCFNVKIIY

SEQ ID NO: 34 - protein, 919 aa, Cetobacterium sp.
MEEIKHKKNKSSIIRVIVSNYDMTGIKEIKVLYQKQGGVDTFNLKTIINLESGNLEIISCKPKEREKYRYEFNCKTEINTISITKKDKVLKKEIRKYSLELYFKNEKKDTVVAKVTDLLKAPDKIEGERNHLRKLSSSTERKLLSKTLCKNYSEISKTPIEEIDSIKIYKIKRFLNYRSNFLIYFALINDFLCAGVKEDDINEVWLIQDKEHTAFLENRIEKITDYIFDKLSKDIENKKNQFEKRIKKYKTSLEELKTETLEKNKTFYIDSIKTKITNLENKITELSLYNSKESLKEDLIKIISIFTNLRHSLMHYDYKSFENLFENIENEELKNLLDLNLFKSIRMSDEFKTKNRTNYLDGTESFTIVKKHQNLKKLYTYYNNLCDKKNGFNTFINSFFVTDGIENTDFKNLIILHFEKEMEEYKKSIEYYKIKISNEKNKSKKEKLKEKIDLLQSELINMREHKNLLKQIYFFDIHNSIKYKELYSERKNLIEQYNLQINGVKDVTAINHINTKLLSLKNKMDKITKQNSLYRLKYKLKIAYSFLMIEFDGDVSKFKNNFDPTNLEKRVEYLDKKEEYLNYTAPKNKFNFAKLEEELQKIQSTSEMGADYLNVSPENNLFKFYILTYIMLPVEFKGDFLGFVKNHYYNIKNVDFMDESLLDENEVDSNKLNEKIENLKDSSFFNKIRLFEKNIKKYEIVKYSVSTQENMKEYFKQLNLDIPYLDYKSTDEIGIFNKNMILPIFKYYQNVFKLCNDIEIHALLALANKKQQNLEYAIYCCSKKNSLNYNELLKTFNRKTYQNLSFIRNKIAHLNYKELFSDLFNNELDLNTKVRCLIEFSQNNKFDQIDLGMNFINDYYMKKTRFIFNQRRLRDLNVPSKEKIIDGKRKQQNDSNNELLKKYGLSRTNIKDIFNKAWY

SEQ ID NO: 35 - protein, 1110 aa, Fusobacterium necrophorum
MKVRYRKQAQLDTFIIKTEIVNNDIFIKSIIEKAREKYRYSFLFDGEEKYHFKNKSSVEIVKNDIFSQTPDNMIRNYKITLKISEKNPRVVEAEIEDLMNSTILKDGRRSARREKSMTERKLIEEKVAENYSLLANCPIEEVDSIKIYKIKRFLTYRSNMLLYFASINSFLCEGIKGKDNETEEIWHLKDNDVRKEKVKENFKNKLIQSTENYNSSLKNQIEEKEKLSSKEFKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTKLLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSEKPFEVFLEELKDKMIGKQLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKKSEKEEILRKRYHLINKKNEQIKDEHEAQSQLYEKILSLQKIFSCDKNNFYRRLKEEKLLFLEKQGKKKISMKEIKDKIASDISDLLGILKKEITRDIKDKLTEKFRYCEEKLLNISFYNHQDKKKEEGIRVFLIRDKNSDNFKFESILDDGSNKIFISKNGKEITIQCCDKVLETLMIEKNTLKISSNGKIISLIPHYSYSIDVKY

SEQ ID NO: 36 - protein, 1228 aa, unidentified
  REGION 1..1228: note = Description of Unknown: Lachnospiraceae bacterium sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 37 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 38 - protein, 1300 aa, Francisella sp.
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 39 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 40 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 41 - protein, 1259 aa, Moraxella bovoculi
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 42 - protein, 1269 aa, Moraxella bovoculi
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 43 - protein, 1306 aa, Thiomicrospira sp.
MGIHGVPAATKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 44 - protein, 1214 aa, Butyrivibrio sp.
MGIHGVPAAYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 45 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 46 - protein, 1224 aa, synthetic construct
  REGION 1..1224: note = Description of Artificial Sequence: Synthetic polypeptide
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 47 - protein, 500 aa, synthetic construct
  REGION 1..500: note = Description of Artificial Sequence: Synthetic polypeptide
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 48 - protein, 507 aa, synthetic construct
  REGION 1..507: note = Description of Artificial Sequence: Synthetic polypeptide
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 49 - protein, 529 aa, synthetic construct
  REGION 1..529: note = Description of Artificial Sequence: Synthetic polypeptide
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 50 - protein, 726 aa, synthetic construct
  REGION 1..726: note = Description of Artificial Sequence: Synthetic polypeptide
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 51 - protein, 517 aa, synthetic construct
  REGION 1..517: note = Description of Artificial Sequence: Synthetic polypeptide
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 52 - protein, 481 aa, synthetic construct
  REGION 1..481: note = Description of Artificial Sequence: Synthetic polypeptide
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 53 - protein, 358 aa, synthetic construct
  REGION 1..358: note = Description of Artificial Sequence: Synthetic polypeptide
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 54 - protein, 507 aa, synthetic construct
  REGION 1..507: note = Description of Artificial Sequence: Synthetic polypeptide
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 55 - protein, 534 aa, synthetic construct
  REGION 1..534: note = Description of Artificial Sequence: Synthetic polypeptide
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 56 - protein, 537 aa, synthetic construct
  REGION 1..537: note = Description of Artificial Sequence: Synthetic polypeptide
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 57 - protein, 540 aa, synthetic construct
  REGION 1..540: note = Description of Artificial Sequence: Synthetic polypeptide
LWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 58 - protein, 542 aa, synthetic construct
  REGION 1..542: note = Description of Artificial Sequence: Synthetic polypeptide
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 59 - protein, 564 aa, synthetic construct
  REGION 1..564: note = Description of Artificial Sequence: Synthetic polypeptide
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 60 - protein, 610 aa, synthetic construct
  REGION 1..610: note = Description of Artificial Sequence: Synthetic polypeptide
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 61 - protein, 369 aa, synthetic construct
  REGION 1..369: note = Description of Artificial Sequence: Synthetic polypeptide
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 62 - protein, 552 aa, synthetic construct
  REGION 1..552: note = Description of Artificial Sequence: Synthetic polypeptide
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 63 - protein, 534 aa, synthetic construct
  REGION 1..534: note = Description of Artificial Sequence: Synthetic polypeptide
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 64 - protein, 577 aa, synthetic construct
  REGION 1..577: note = Description of Artificial Sequence: Synthetic polypeptide
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 65 - protein, 613 aa, synthetic construct
  REGION 1..613: note = Description of Artificial Sequence: Synthetic polypeptide
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 66 - protein, 615 aa, synthetic construct
  REGION 1..615: note = Description of Artificial Sequence: Synthetic polypeptide
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 67 - protein, 775 aa, synthetic construct
  REGION 1..775: note = Description of Artificial Sequence: Synthetic polypeptide
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 68 - protein, 777 aa, synthetic construct
  REGION 1..777: note = Description of Artificial Sequence: Synthetic polypeptide
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 69 - protein, 610 aa, synthetic construct
  REGION 1..610: note = Description of Artificial Sequence: Synthetic polypeptide
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 70 - protein, 632 aa, synthetic construct
  REGION 1..632: note = Description of Artificial Sequence: Synthetic polypeptide
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 71 - protein, 625 aa, synthetic construct
  REGION 1..625: note = Description of Artificial Sequence: Synthetic polypeptide
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 72 - protein, 517 aa, synthetic construct
  REGION 1..517: note = Description of Artificial Sequence: Synthetic polypeptide
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 73 - protein, 410 aa, synthetic construct
  REGION 1..410: note = Description of Artificial Sequence: Synthetic polypeptide
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 74 - protein, 486 aa, synthetic construct
  REGION 1..486: note = Description of Artificial Sequence: Synthetic polypeptide
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 75 - protein, 602 aa, synthetic construct
  REGION 1..602: note = Description of Artificial Sequence: Synthetic polypeptide
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 76 - protein, 494 aa, synthetic construct
  REGION 1..494: note = Description of Artificial Sequence: Synthetic polypeptide
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 77 - protein, 526 aa, synthetic construct
  REGION 1..526: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 78 - protein, 649 aa, synthetic construct
  REGION 1..649: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 79 - protein, 414 aa, synthetic construct
  REGION 1..414: note = Description of Artificial Sequence: Synthetic polypeptide
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 80 - protein, 413 aa, synthetic construct
  REGION 1..413: note = Description of Artificial Sequence: Synthetic polypeptide
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 81 - protein, 449 aa, synthetic construct
  REGION 1..449: note = Description of Artificial Sequence: Synthetic polypeptide
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 82 - protein, 711 aa, synthetic construct
  REGION 1..711: note = Description of Artificial Sequence: Synthetic polypeptide
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 83 - protein, 574 aa, synthetic construct
  REGION 1..574: note = Description of Artificial Sequence: Synthetic polypeptide
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 84 - protein, 400 aa, synthetic construct
  REGION 1..400: note = Description of Artificial Sequence: Synthetic polypeptide
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 85 - protein, 743 aa, synthetic construct
  REGION 1..743: note = Description of Artificial Sequence: Synthetic polypeptide
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 86 - protein, 769 aa, synthetic construct
  REGION 1..769: note = Description of Artificial Sequence: Synthetic polypeptide
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 87 - protein, 666 aa, synthetic construct
  REGION 1..666: note = Description of Artificial Sequence: Synthetic polypeptide
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 88 - protein, 564 aa, synthetic construct
  REGION 1..564: note = Description of Artificial Sequence: Synthetic polypeptide
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 89 - protein, 565 aa, synthetic construct
  REGION 1..565: note = Description of Artificial Sequence: Synthetic polypeptide
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 90 - protein, 499 aa, synthetic construct
  REGION 1..499: note = Description of Artificial Sequence: Synthetic polypeptide
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 91 - protein, 560 aa, synthetic construct
  REGION 1..560: note = Description of Artificial Sequence: Synthetic polypeptide
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 92 - protein, 404 aa, synthetic construct
  REGION 1..404: note = Description of Artificial Sequence: Synthetic polypeptide
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 93 - protein, 392 aa, synthetic construct
  REGION 1..392: note = Description of Artificial Sequence: Synthetic polypeptide
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 94 - protein, 500 aa, synthetic construct
  REGION 1..500: note = Description of Artificial Sequence: Synthetic polypeptide
KEPLNIGKTAKAVFKEIDPTSLNRAANYDASIELNCKECKFKPFKNVKRYEFNFYNNWYRCNPNSCLQSTYKAQVRKVEIGYEKLKNEILTQMQYYPWFGRLYQNFFHDERDKMTSLDEIQVIGVQNKVFFNTVEKAWREIIKKRFKDNKETMETIPELKHAAGHGKRKLSNKSLLRRRFAFVQKSFKFVDNSDVSYRSFSNNIACVLPSRIGVDLGGVISRNPKREYIPQEISFNAFWKQHEGLKKGRNIEIQSVQYKGETVKRIEADTGEDKAWGKNRQRRFTSLILKLVPKQGGKKVWKYPEKRNEGNYEYFPIPIEFILDSGETSIRFGGDEGEAGKQKHLVIPFNDSKATPLASQQTLLENSRFNAEVKSCIGLAIYANYFYGYARNYVISSIYHKNSKNGQAITAIYLESIAHNYVKAIERQLQNLLLNLRDFSFMESHKKELKKYFGGDLEGTGGAQKRREKEEKIEKEIEQSYLPRLIRLSLTKMVTKQVEM

SEQ ID NO: 95 - protein, 507 aa, synthetic construct
  REGION 1..507: note = Description of Artificial Sequence: Synthetic polypeptide
ELIVNENKDPLNIGKTAKAVFKEIDPTSINRAANYDASIELACKECKFKPFNNTKRHDFSFYSNWHRCSPNSCLQSTYRAKIRKTEIGYEKLKNEILNQMQYYPWFGRLYQNFFNDQRDKMTSLDEIQVTGVQNKIFFNTVEKAWREIIKKRFRDNKETMRTIPDLKNKSGHGSRKLSNKSLLRRRFAFAQKSFKLVDNSDVSYRAFSNNVACVLPSKIGVDIGGIINKDLKREYIPQEITFNVFWKQHDGLKKGRNIEIHSVQYKGEIVKRIEADTGEDKAWGKNRQRRFTSLILKITPKQGGKKIWKFPEKKNASDYEYFPIPIEFILDNGDASIKFGGEEGEVGKQKHLLIPFNDSKATPLSSKQMLLETSRFNAEVKSTIGLALYANYFVSYARNYVIKSTYHKNSKKGQIVTEIYLESISQNFVRAIQRQLQSLMLNLKDWGFMQTHKKELKKYFGSDLEGSKGGQKRREKEEKIEKEIEASYLPRLIRLSLTKSVTKAEEM

SEQ ID NO: 96 - protein, 529 aa, synthetic construct
  REGION 1..529: note = Description of Artificial Sequence: Synthetic polypeptide
PEEKTSKLKPNSINLAANYDANEKFNCKECKFHPFKNKKRYEFNFYNNLHGCKSCTKSTNNPAVKRIEIGYQKLKFEIKNQMEAYPWFGRLRINFYSDEKRKMSELNEMQVTGVKNKIFFDAIECAWREILKKRFRESKETLITIPKLKNKAGHGARKHRNKKLLIRRRAFMKKNFHFLDNDSISYRSFANNIACVLPSKVGVDIGGIISPDVGKDIKPVDISLNLMWASKEGIKSGRKVEIYSTQYDGNMVKKIEAETGEDKSWGKNRKRRQTSLLLSIPKPSKQVQEFDFKEWPRYKDIEKKVQWRGFPIKIIFDSNHNSIEFGTYQGGKQKVLPIPFNDSKTTPLGSKMNKLEKLRFNSKIKSRLGSAIAANKFLEAARTYCVDSLYHEVSSANAIGKGKIFIEYYLEILSQNYIEAAQKQLQRFIESIEQWFVADPFQGRLKQYFKDDLKRAKCFLCANREVQTTCYAAVKLHKSCAEKVKDKNKELAIKERNNKEDAVIKEVEASNYPRVIRLKLTKTITNKAM

SEQ ID NO: 97 - protein, 726 aa, synthetic construct
  REGION 1..726: note = Description of Artificial Sequence: Synthetic polypeptide
SESENKIIEQYYAFLYSFRDKYEKPEFKNRGDIKRKLQNKWEDFLKEQNLKNDKKLSNYIFSNRNFRRSYDREEENEEGIDEKKSKPKRINCFEKEKNLKDQYDKDAINASANKDGAQKWGCFECIFFPMYKIESGDPNKRIIINKTRFKLFDFYLNLKGCKSCLRSTYHPYRSNVYIESNYDKLKREIGNFLQQKNIFQRMRKAKVSEGKYLTNLDEYRLSCVAMHFKNRWLFFDSIQKVLRETIKQRLKQMRESYDEQAKTKRSKGHGRAKYEDQVRMIRRRAYSAQAHKLLDNGYITLFDYDDKEINKVCLTAINQEGFDIGGYLNSDIDNVMPPIEISFHLKWKYNEPILNIESPFSKAKISDYLRKIREDLNLERGKEGKARSKKNVRRKVLASKGEDGYKKIFTDFFSKWKEELEGNAMERVLSQSSGDIQWSKKKRIHYTTLVLNINLLDKKGVGNLKYYEIAEKTKILSFDKNENKFWPITIQVLLDGYEIGTEYDEIKQLNEKTSKQFTIYDPNTKIIKIPFTDSKAVPLGMLGINIATLKTVKKTERDIKVSKIFKGGLNSKIVSKIGKGIYAGYFPTVDKEILEEVEEDTLDNEFSSKSQRNIFLKSIIKNYDKMLKEQLFDFYSFLVRNDLGVRFLTDRELQNIEDESFNLEKRFFETDRDRIARWFDNTNTDDGKEKFKKLANEIVDSYKPRLIRLPVVRVIKRIQPVKQREM

SEQ ID NO: 98 - protein, 517 aa, synthetic construct
  REGION 1..517: note = Description of Artificial Sequence: Synthetic polypeptide
KYSTRDFSELNEIQVTACKQDEFFKVIQNAWREIIKKRFLENRENFIEKKIFKNKKGRGKRQESDKTIQRNRASVMKNFQLIENEKIILRAPSGHVACVFPVKVGLDIGGFKTDDLEKNIFPPRTITINVFWKNRDRQRKGRKLEVWGIKARTKLIEKVHKWDKLEEVKKKRLKSLEQKQEKSLDNWSEVNNDSFYKVQIDELQEKIDKSLKGRTMNKILDNKAKESKEAEGLYIEWEKDFEGEMLRRIEASTGGEEKWGKRRQRRHTSLLLDIKNNSRGSKEIINFYSYAKQGKKEKKIEFFPFPLTITLDAEEESPLNIKSIPIEDKNATSKYFSIPFTETRATPLSILGDRVQKFKTKNISGAIKRNLGSSISSCKIVQNAETSAKSILSLPNVKEDNNMEIFINTMSKNYFRAMMKQMESFIFEMEPKTLIDPYKEKAIKWFEVAASSRAKRKLKKLSKADIKKSELLLSNTEEFEKEKQEKLEALEKEIEEFYLPRIVRLQLTKTILETPVM

SEQ ID NO: 99 - protein, 481 aa, synthetic construct
  REGION 1..481: note = Description of Artificial Sequence: Synthetic polypeptide
KKLQLLGHKILLKEYDPNAVNAAANFETSTAELCGQCKMKPFKNKRRFQYTFGKNYHGCLSCIQNVYYAKKRIVQIAKEELKHQLTDSIASIPYKYTSLFSNTNSIDELYILKQERAAFFSNTNSIDELYITGIENNIAFKVISAIWDEIIKKRRQRYAESLTDTGTVKANRGHGGTAYKSNTRQEKIRALQKQTLHMVTNPYISLARYKNNYIVATLPRTIGMHIGAIKDRDPQKKLSDYAINFNVFWSDDRQLIELSTVQYTGDMVRKIEAETGENNKWGENMKRTKTSLLLEILTKKTTDELTFKDWAFSTKKEIDSVTKKTYQGFPIGIIFEGNESSVKFGSQNYFPLPFDAKITPPTAEGFRLDWLRKGSFSSQMKTSYGLAIYSNKVTNAIPAYVIKNMFYKIARAENGKQIKAKFLKKYLDIAGNNYVPFIIMQHYRVLDTFEEMPISQPKVIRLSLTKTQHIIIKKDKTDSKM

SEQ ID NO: 100 - protein, 534 aa, synthetic construct
  REGION 1..534: note = Description of Artificial Sequence: Synthetic polypeptide
NTSNLINLGKKAINISANYDANLEVGCKNCKFLSSNGNFPRQTNVKEGCHSCEKSTYEPSIYLVKIGERKAKYDVLDSLKKFTFQSLKYQSKKSMKSRNKKPKELKEFVIFANKNKAFDVIQKSYNHLILQIKKEINRMNSKKRKKNHKRRLFRDREKQLNKLRLIESSNLFLPRENKGNNHVFTYVAIHSVGRDIGVIGSYDEKLNFETELTYQLYFNDDKRLLYAYKPKQNKIIKIKEKLWNLRKEKEPLDLEYEKPLNKSITFSIKNDNLFKVSKDLMLRRAKFNIQGKEKLSKEERKINRDLIKIKGLVNSMSYGRFDELKKEKNIWSPHIYREVRQKEIKPCLIKNGDRIEIFEQLKKKMERLRRFREKRQKKISKDLIFAERIAYNFHTKSIKNTSNKINIDQEAKRGKASYMRKRIGYETFKNKYCEQCLSKGNVYRNVQKGCSCFENPFDWIKKGDENLLPKKNEDLRVKGAFRDEALEKQIVKIAFNIAKGYEDFYDNLGESTEKDLKLKFKVGTTINEQESLKL

SEQ ID NO: 101 - protein, 537 aa, synthetic construct
  REGION 1..537: note = Description of Artificial Sequence: Synthetic polypeptide
TSNPIKLGKKAINISANYDSNLQIGCKNCKFLSYNGNFPRQTNVKEGCHSCEKSTYEPPVYTVRIGERRSKYDVLDSLKKFIFLSLKYRQSKKMKTRSKGIRGLEEFVISANLKKAMDVIQKSYRHLILNIKNEIVRMNGKKRNKNHKRLLFRDREKQLNKLRLIEGSSFFKPPTVKGDNSIFTCVAIHNIGRDIGIAGDYFDKLEPKIELTYQLYYEYNPKKESEINKRLLYAYKPKQNKIIEIKEKLWNLRKEKSPLDLEYEKPLTKSITFLVKRDGVFRISKDLMLRKAKFIIQGKEKLSKEERKINRDLIKIKSNIISLTYGRFDELKKDKTIWSPHIFRDVKQGKITPCIERKGDRMDIFQQLRKKSERLRENRKKRQKKISKDLIFAERIAYNFHTKSIKNTSNLINIKHEAKRGKASYMRKRIGNETFRIKYCEQCFPKNNVYKNVQKGCSCFEDPFEYIKKGNEDLIPNKNQDLKAKGAFRDDALEKQIIKVAFNIAKGYEDFYENLKKTTEKDIRLKFKVGTIISEEM

SEQ ID NO: 102 - protein, 541 aa, synthetic construct
  REGION 1..541: note = Description of Artificial Sequence: Synthetic polypeptide
NNSINLSKKAINISANYDANLQVRCKNCKFLSSNGNFPRQTDVKEGCHSCEKSTYEPPVYDVKIGEIKAKYEVLDSLKKFTFQSLKYQLSKSMKFRSKKIKELKEFVIFAKESKALNVINRSYKHLILNIKNDINRMNSKKRIKNHKGRLFLDRQKQLSKLKLIEGSSFFVPAKNVGNKSVFTCVAIHSIGRDIGIAGLYDSFTKPVNEITYQIFFSGERRLLYAYKPKQLKILSIKENLWSLKNEKKPLDLLYEKPLGKNLNFNVKGGDLFRVSKDLMIRNAKFNVHGRQRLSDEERLINRNFIKIKGEVVSLSYGRFEELKKDRKLWSPHIFKDVRQNKIKPCLVMQGQRIDIFEQLKRKLELLKKIRKSRQKKLSKDLIFGERIAYNFHTKSIKNTSNKINIDSDAKRGRASYMRKRIGNETFKLKYCDVCFPKANVYRRVQNGCSCSENPYNYIKKGDKDLLPKKDEGLAIKGAFRDEKLNKQIIKVAFNIAKGYEDFYDDLKKRTEKDVDLKFKIGTTVLDQKPMEIFDGIVITWL

SEQ ID NO: 103 - protein, 542 aa, synthetic construct
  REGION 1..542: note = Description of Artificial Sequence: Synthetic polypeptide
LLTTVVETNNLAKKAINVAANFDANIDRQYYRCTPNLCRFIAQSPRETKEKDAGCSSCTQSTYDPKVYVIKIGKLLAKYEILKSLKRFLFMNRYFKQKKTERAQQKQKIGTELNEMSIFAKATNAMEVIKRATKHCTYDIIPETKSLQMLKRRRHRVKVRSLLKILKERRMKIKKIPNTFIEIPKQAKKNKSDYYVAAALKSCGIDVGLCGAYEKNAEVEAEYTYQLYYEYKGNSSTKRILYCYNNPQKNIREFWEAFYIQGSKSHVNTPGTIRLKMEKFLSPITIESEALDFRVWNSDLKIRNGQYGFIKKRSLGKEAREIKKGMGDIKRKIGNLTYGKSPSELKSIHVYRTERENPKKPRAARKKEDNFMEIFEMQRKKDYEVNKKRRKEATDAAKIMDFAEEPIRHYHTNNLKAVRRIDMNEQVERKKTSVFLKRIMQNGYRGNYCRKCIKAPEGSNRDENVLEKNEGCLDCIGSEFIWKKSSKEKKGLWHTNRLLRRIRLQCFTTAKAYENFYNDLFEKKESSLDIIKLKVSITTKSM

SEQ ID NO: 104 - protein, 564 aa, synthetic construct
  REGION 1..564: note = Description of Artificial Sequence: Synthetic polypeptide
ASTMNLAKQAINFAANYDSNLEIGCKGCKFMSTWSKKSNPKFYPRQNNQANKCHSCTYSTGEPEVPIIEIGERAAKYKIFTALKKFVFMSVAYKERRRQRFKSKKPKELKELAICSNREKAMEVIQKSVVHCYGDVKQEIPRIRKIKVLKNHKGRLFYKQKRSKIKIAKLEKGSFFKTFIPKVHNNGCHSCHEASLNKPILVTTALNTIGADIGLINDYSTIAPTETDISWQVYYEFIPNGDSEAVKKRLLYFYKPKGALIKSIRDKYFKKGHENAVNTGFFKYQGKIVKGPIKFVNNELDFARKPDLKSMKIKRAGFAIPSAKRLSKEDREINRESIKIKNKIYSLSYGRKKTLSDKDIIKHLYRPVRQKGVKPLEYRKAPDGFLEFFYSLKRKERRLRKQKEKRQKDMSEIIDAADEFAWHRHTGSIKKTTNHINFKSEVKRGKVPIMKKRIANDSFNTRHCGKCVKQGNAINKYYIEKQKNCFDCNSIEFKWEKAALEKKGAFKLNKRLQYIVKACFNVAKAYESFYEDFRKGEEESLDLKFKIGTTTTLKQYPQNKARAM

SEQ ID NO: 105 - protein, 610 aa, synthetic construct
  REGION 1..610: note = Description of Artificial Sequence: Synthetic polypeptide
HSHNLMLTKLGKQAINFAANYDANLEIGCKNCKFLSYSPKQANPKKYPRQTDVHEDGNIACHSCMQSTKEPPVYIVPIGERKSKYEILTSLNKFTFLALKYKEKKRQAFRAKKPKELQELAIAFNKEKAIKVIDKSIQHLILNIKPEIARIQRQKRLKNRKGKLLYLHKRYAIKMGLIKNGKYFKVGSPKKDGKKLLVLCALNTIGRDIGIIGNIEENNRSETEITYQLYFDCLDANPNELRIKEIEYNRLKSYERKIKRLVYAYKPKQTKILEIRSKFFSKGHENKVNTGSFNFENPLNKSISIKVKNSAFDFKIGAPFIMLRNGKFHIPTKKRLSKEEREINRTLSKIKGRVFRLTYGRNISEQGSKSLHIYRKERQHPKLSLEIRKQPDSFIDEFEKLRLKQNFISKLKKQRQKKLADLLQFADRIAYNYHTSSLEKTSNFINYKPEVKRGRTSYIKKRIGNEGFEKLYCETCIKSNDKENAYAVEKEELCFVCKAKPFTWKKTNKDKLGIFKYPSRIKDFIRAAFTVAKSYNDFYENLKKKDLKNEIFLKFKIGLILSHEKKNHISIAKSVAEDERISGKSIKNILNKSIKLEKNCYSCFFHKEDM

SEQ ID NO: 106 - protein, 552 aa, synthetic construct
  REGION 1..552: note = Description of Artificial Sequence: Synthetic polypeptide
SLERVIDKRNLAKKAINIAANFDANINKGFYRCETNQCMFIAQKPRKTNNTGCSSCLQSTYDPVIYVVKVGEMLAKYEILKSLKRFVFMNRSFKQKKTEKAKQKERIGGELNEMSIFANAALAMGVIKRAIRHCHVDIRPEINRLSELKKTKHRVAAKSLVKIVKQRKTKWKGIPNSFIQIPQKARNKDADFYVASALKSGGIDIGLCGTYDKKPHADPRWTYQLYFDTEDESEKRLLYCYNDPQAKIRDFWKTFYERGNPSMVNSPGTIEFRMEGFFEKMTPISIESKDFDFRVWNKDLLIRRGLYEIKKRKNLNRKAREIKKAMGSVKRVLANMTYGKSPTDKKSIPVYRVEREKPKKPRAVRKEENELADKLENYRREDFLIRNRRKREATEIAKIIDAAEPPIRHYHTNHLRAVKRIDLSKPVARKNTSVFLKRIMQNGYRGNYCKKCIKGNIDPNKDECRLEDIKKCICCEGTQNIWAKKEKLYTGRINVLNKRIKQMKLECFNVAKAYENFYDNLAALKEGDLKVLKLKVSIPALNPEASDPEEDM

SEQ ID NO: 107 - protein, 534 aa, synthetic construct
  REGION 1..534: note = Description of Artificial Sequence: Synthetic polypeptide
NASINLGKRAINLSANYDSNLVIGCKNCKFLSFNGNFPRQTNVREGCHSCDKSTYAPEVYIVKIGERKAKYDVLDSLKKFTFQSLKYQIKKSMRERSKKPKELLEFVIFANKDKAFNVIQKSYEHLILNIKQEINRMNGKKRIKNHKKRLFKDREKQLNKLRLIGSSSLFFPRENKGDKDLFTYVAIHSVGRDIGVAGSYESHIEPISDLTYQLFINNEKRLLYAYKPKQNKIIELKENLWNLKKEKKPLDLEFTKPLEKSITFSVKNDKLFKVSKDLMLRQAKFNIQGKEKLSKEERQINRDFSKIKSNVISLSYGRFEELKKEKNIWSPHIYREVKQKEIKPCIVRKGDRIELFEQLKRKMDKLKKFRKERQKKISKDLNFAERIAYNFHTKSIKNTSNKINIDQEAKRGKASYMRKRIGNESFRKKYCEQCFSVGNVYHNVQNGCSCFDNPIELIKKGDEGLIPKGKEDRKYKGALRDDNLQMQIIRVAFNIAKGYEDFYNNLKEKTEKDLKLKFKIGTTISTQESNNKEM

SEQ ID NO: 108 - protein, 577 aa, synthetic construct
  REGION 1..577: note = Description of Artificial Sequence: Synthetic polypeptide
SNLIKLGKQAINFAANYDANLEVGCKNCKFLSSTNKYPRQTNVHLDNKMACRSCNQSTMEPAIYIVRIGEKKAKYDIYNSLTKFNFQSLKYKAKRSQRFKPKQPKELQELSIAVRKEKALDIIQKSIDHLIQDIRPEIPRIKQQKRYKNHVGKLFYLQKRRKNKLNLIGKGSFFKVFSPKEKKNELLVICALTNIGRDIGLIGNYNTIINPLFEVTYQLYYDYIPKKNNKNVQRRLLYAYKSKNEKILKLKEAFFKRGHENAVNLGSFSYEKPLEKSLTLKIKNDKDDFQVSPSLRIRTGRFFVPSKRNLSRQEREINRRLVKIKSKIKNMTYGKFETARDKQSVHIFRLERQKEKLPLQFRKDEKEFMEEFQKLKRRTNSLKKLRKSRQKKLADLLQLSEKVVYNNHTGTLKKTSNFLNFSSSVKRGKTAYIKELLGQEGFETLYCSNCINKGQKTRYNIETKEKCFSCKDVPFVWKKKSTDKDRKGAFLFPAKLKDVIKATFTVAKAYEDFYDNLKSIDEKKPYIKFKIGLILAHVRHEHKARAKEEAGQKNIYNKPIKIDKNCKECFFFKEEAM

SEQ ID NO: 109 - protein, 613 aa, synthetic construct
  REGION 1..613: note = Description of Artificial Sequence: Synthetic polypeptide
NTTRKKFRKRTGFPQSDNIKLAYCSAIVRAANLDADIQKKHNQCNPNLCVGIKSNEQSRKYEHSDRQALLCYACNQSTGAPKVDYIQIGEIGAKYKILQMVNAYDFLSLAYNLTKLRNGKSRGHQRMSQLDEVVIVADYEKATEVIKRSINHLLDDIRGQLSKLKKRTQNEHITEHKQSKIRRKLRKLSRLLKRRRWKWGTIPNPYLKNWVFTKKDPELVTVALLHKLGRDIGLVNRSKRRSKQKLLPKVGFQLYYKWESPSLNNIKKSKAKKLPKRLLIPYKNVKLFDNKQKLENAIKSLLESYQKTIKVEFDQFFQNRTEEIIAEEQQTLERGLLKQLEKKKNEFASQKKALKEEKKKIKEPRKAKLLMEESRSLGFLMANVSYALFNTTIEDLYKKSNVVSGCIPQEPVVVFPADIQNKGSLAKILFAPKDGFRIKFSGQHLTIRTAKFKIRGKEIKILTKTKREILKNIEKLRRVWYREQHYKLKLFGKEVSAKPRFLDKRKTSIERRDPNKLADQTDDRQAELRNKEYELRHKQHKMAERLDNIDTNAQNLQTLSFWVGEADKPPKLDEKDARGFGVRTCISAWKWFMEDLLKKQEEDPLLKLKLSIM

SEQ ID NO: 110 - protein, 615 aa, synthetic construct
  REGION 1..615: note = Description of Artificial Sequence: Synthetic polypeptide
PKKPKFQKRTGFPQPDNLRKEYCLAIVRAANLDADFEKKCTKCEGIKTNKKGNIVKGRTYNSADKDNLLCYACNISTGAPAVDYVFVGALEAKYKILQMVKAYDFHSLAYNLAKLWKGRGRGHQRMGGLNEVVIVSNNEKALDVIEKSLNHFHDEIRGELSRLKAKFQNEHLHVHKESKLRRKLRKISRLLKRRRWKWDVIPNSYLRNFTFTKTRPDFISVALLHRVGRDIGLVTKTKIPKPTDLLPQFGFQIYYTWDEPKLNKLKKSRLRSEPKRLLVPYKKIELYKNKSVLEEAIRHLAEVYTEDLTICFKDFFETQKRKFVSKEKESLKRELLKELTKLKKDFSERKTALKRDRKEIKEPKKAKLLMEESRSLGFLAANTSYALFNLIAADLYTKSKKACSTKLPRQLSTILPLEIKEHKSTTSLAIKPEEGFKIRFSNTHLSIRTPKFKMKGADIKALTKRKREILKNATKLEKSWYGLKHYKLKLYGKEVAAKPRFLDKRNPSIDRRDPKELMEQIENRRNEVKDLEYEIRKGQHQMAKRLDNVDTNAQNLQTKSFWVGEADKPPELDSMEAKKLGLRTCISAWKWFMKDLVLLQEKSPNLKLKLSLTEM

SEQ ID NO: 111 - protein, 775 aa, synthetic construct
  REGION 1..775: note = Description of Artificial Sequence: Synthetic polypeptide
KFSKRQEGFLIPDNIDLYKCLAIVRSANLDADVQGHKSCYGVKKNGTYRVKQNGKKGVKEKGRKYVFDLIAFKGNIEKIPHEAIEEKDQGRVIVLGKFNYKLILNIEKNHNDRASLEIKNKIKKLVQISSLETGEFLSDLLSGKIGIDEVYGIIEPDVFSGKELVCKACQQSTYAPLVEYMPVGELDAKYKILSAIKGYDFLSLAYNLSRNRANKKRGHQKLGGGELSEVVISANYDKALNVIKRSINHYHVEIKPEISKLKKKMQNEPLKVMKQARIRRELHQLSRKVKRLKWKWGMIPNPELQNIIFEKKEKDFVSYALLHTLGRDIGLFKDTSMLQVPNISDYGFQIYYSWEDPKLNSIKKIKDLPKRLLIPYKRLDFYIDTILVAKVIKNLIELYRKSYVYETFGEEYGYAKKAEDILFDWDSINLSEGIEQKIQKIKDEFSDLLYEARESKRQNFVESFENILGLYDKNFASDRNSYQEKIQSMIIKKQQENIEQKLKREFKEVIERGFEGMDQNKKYYKVLSPNIKGGLLYTDTNNLGFFRSHLAFMLLSKISDDLYRKNNLVSKGGNKGILDQTPETMLTLEFGKSNLPNISIKRKFFNIKYNSSWIGIRKPKFSIKGAVIREITKKVRDEQRLIKSLEGVWHKSTHFKRWGKPRFNLPRHPDREKNNDDNLMESITSRREQIQLLLREKQKQQEKMAGRLDKIDKEIQNLQTANFQIKQIDKKPALTEKSEGKQSVRNALSAWKWFMEDLIKYQKRTPILQLKLAKM

SEQ ID NO: 112 - protein, 777 aa, synthetic construct
  REGION 1..777: note = Description of Artificial Sequence: Synthetic polypeptide
KFSKRQEGFVIPENIGLYKCLAIVRSANLDADVQGHVSCYGVKKNGTYVLKQNGKKSIREKGRKYASDLVAFKGDIEKIPFEVIEEKKKEQSIVLGKFNYKLVLDVMKGEKDRASLTMKNKSKKLVQVSSLGTDEFLLTLLNEKFGIEEIYGIIEPEVFSGKKLVCKACQQSTYAPLVEYMPVGELDSKYKILSAIKGYDFLSLAYNLARHRSNKKRGHQKLGGGELSEVVISANNAKALNVIKRSLNHYYSEIKPEISKLRKKMQNEPLKVGKQARMRRELHQLSRKVKRLKWKWGKIPNLELQNITFKESDRDFISYALLHTLGRDIGMFNKTEIKMPSNILGYGFQIYYDWEEPKLNTIKKSKNTPKRILIPYKKLDFYNDSILVARAIKELVGLFQESYEWEIFGNEYNYAKEAEVELIKLDEESINGNVEKKLQRIKENFSNLLEKAREKKRQNFIESFESIARLYDESFTADRNEYQREIQSFIIEKQKQSIEKKLKNEFKKIVEKKFNEQEQGKKHYRVLNPTIINEFLPKDKNNLGFLRSKIAFILLSKISDDLYKKSNAVSKGGEKGIIKQQPETILDLEFSKSKLPSINIKKKLFNIKYTSSWLGIRKPKFNIKGAKIREITRRVRDVQRTLKSAESSWYASTHFRRWGFPRFNQPRHPDKEKKSDDRLIESITLLREQIQILLREKQKGQKEMAGRLDDVDKKIQNLQTANFQIKQTGDKPALTEKSAGKQSFRNALSAWKWFMENLLKYQNKTPDLKLKIARTVM

SEQ ID NO: 113 - protein, 610 aa, synthetic construct
  REGION 1..610: note = Description of Artificial Sequence: Synthetic polypeptide
KWIEPNNIDFNKCLAITRSANLDADVQGHKMCYGIKTNGTYKAIGKINKKHNTGIIEKRRTYVYDLIVTKEKNEKIVKKTDFMAIDEEIEFDEKKEKLLKKYIKAEVLGTGELIRKDLNDGEKFDDLCSIEEPQAFRRSELVCKACNQSTYASDIRYIPIGEIEAKYKILKAIKGYDFLSLKYNLGRLRDSKKRGHQKMGQGELKEFVICANKEKALDVIKRSLNHYLNEVKDEISRLNKKMQNEPLKVNDQARWRRELNQISRRLKRLKWKWGEIPNPELKNLIFKSSRPEFVSYALIHTLGRDIGLINETELKPNNIQEYGFQIYYKWEDPELNHIKKVKNIPKRFIIPYKNLDLFGKYTILSRAIEGILKLYSSSFQYKSFKDPNLFAKEGEKKITNEDFELGYDEKIKKIKDDFKSYKKALLEKKKNTLEDSLNSILSVYEQSLLTEQINNVKKWKEGLLKSKESIHKQKKIENIEDIISRIEELKNVEGWIRTKERDIVNKEETNLKREIKKELKDSYYEEVRKDFSDLKKGEESEKKPFREEPKPIVIKDYIKFDVLPGENSALGFFLSHLSFNLFDSIQYELFEKSRLSSSKHPQIPETILDL

SEQ ID NO: 114 - protein, 632 aa, synthetic construct
  REGION 1..632: note = Description of Artificial Sequence: Synthetic polypeptide
FRKFVKRSGAPQPDNLNKYKCIAIVRAANLDADIMSNESSNCVMCKGIKMNKRKTAKGAAKTTELGRVYAGQSGNLLCTACTKSTMGPLVDYVPIGRIRAKYTILRAVKEYDFLSLAYNLARTRVSKKGGRQKMHSLSELVIAAEYEIAWNIIKSSVIHYHQETKEEISGLRKKLQAEHIHKNKEARIRREMHQISRRIKRLKWKWHMIPNSELHNFLFKQQDPSFVAVALLHTLGRDIGMINKPKGSAKREFIPEYGFQIYYKWMNPKLNDINKQKYRKMPKRSLIPYKNLNVFGDRELIENAMHKLLKLYDENLEVKGSKFFKTRVVAISSKESEKLKRDLLWKGELAKIKKDFNADKNKMQELFKEVKEPKKANALMKQSRNMGFLLQNISYGALGLLANRMYEASAKQSKGDATKQPSIVIPLEMEFGNAFPKLLLRSGKFAMNVSSPWLTIRKPKFVIKGNKIKNITKLMKDEKAKLKRLETSYHRATHFRPTLRGSIDWDSPYFSSPKQPNTHRRSPDRLSADITEYRGRLKSVEAELREGQRAMAKKLDSVDMTASNLQTSNFQLEKGEDPRLTEIDEKGRSIRNCISSWKKFMEDLMKAQEANPVIKIKIALKDESSVLSEDSM

SEQ ID NO: 115 - protein, 625 aa, synthetic construct
  REGION 1..625: note = Description of Artificial Sequence: Synthetic polypeptide
KFHPENLNKSYCLAIVRAANLDADIQGHINCIGIKSNKSDRNYENKLESLQNVELLCKACTKSTYKPNINSVPVGEKKAKYSILSEIKKYDFNSLVYNLKKYRKGKSRGHQKLNELRELVITSEYKKALDVINKSVNHYLVNIKNKMSKLKKILQNEHIHVGTLARIRRERNRISRKLDHYRKKWKFVPNKILKNYVFKNQSPDFVSVALLHKLGRDIGLITKTAILQKSFPEYSLQLYYKYDTPKLNYLKKSKFKSLPKRILISYKYPKFDINSNYIEESIDKLLKLYEESPIYKNNSKIIEFFKKSEDNLIKSENDSLKRGIMKEFEKVTKNFSSKKKKLKEELKLKNEDKNSKMLAKVSRPIGFLKAYLSYMLFNIISNRIFEFSRKSSGRIPQLPSCIINLGNQFENFKNELQDSNIGSKKNYKYFCNLLLKSSGFNISYEEEHLSIKTPNFFINGRKLKEITSEKKKIRKENEQLIKQWKKLTFFKPSNLNGKKTSDKIRFKSPNNPDIERKSEDNIVENIAKVKYKLEDLLSEQRKEFNKLAKKHDGVDVEAQCLQTKSFWIDSNSPIKKSLEKKNEKVSVKKKMKAIRSCISAWKWFMADLIEAQKETPMIKLKLALM

SEQ ID NO: 116 - protein, 517 aa, synthetic construct
  REGION 1..517: note = Description of Artificial Sequence: Synthetic polypeptide
TTLVPSHLAGIEVMDETTSRNEDMIQKETSRSNEDENYLGVKNKCGINVHKSGRGSSKHEPNMPPEKSGEGQMPKQDSTEMQQRFDESVTGETQVSAGATASIKTDARANSGPRVGTARALIVKASNLDRDIKLGCKPCEYIRSELPMGKKNGCNHCEKSSDIASVPKVESGFRKAKYELVRRFESFAADSISRHLGKEQARTRGKRGKKDKKEQMGKVNLDEIAILKNESLIEYTENQILDARSNRIKEWLRSLRLRLRTRNKGLKKSKSIRRQLITLRRDYRKWIKPNPYRPDEDPNENSLRLHTKLGVDIGVQGGDNKRMNSDDYETSFSITWRDTATRKICFTKPKGLLPRHMKFKLRGYPELILYNEELRIQDSQKFPLVDWERIPIFKLRGVSLGKKKVKALNRITEAPRLVVAKRIQVNIESKKKKVLTRYVYNDKSINGRLVKAEDSNKDPLLEFKKQAEEINSDAKYYENQEIAKNYLWGCEGLHKNLLEEQTKNPYLAFKYGFLNIV

SEQ ID NO: 117 - protein, 410 aa, synthetic construct
  REGION 1..410: note = Description of Artificial Sequence: Synthetic polypeptide
LDFKRTCSQELVLLPEIEGLKLSGTQGVTSLAKKLINKAANVDRDESYGCHHCIHTRTSLSKPVKKDCNSCNQSTNHPAVPITLKGYKIAFYELWHRFTSWAVDSISKALHRNKVMGKVNLDEYAVVDNSHIVCYAVRKCYEKRQRSVRLHKRAYRCRAKHYNKSQPKVGRIYKKSKRRNARNLKKEAKRYFQPNEITNGSSDALFYKIGVDLGIAKGTPETEVKVDVSICFQVYYGDARRVLRVRKMDELQSFHLDYTGKLKLKGIGNKDTFTIAKRNESLKWGSTKYEVSRAHKKFKPFGKKGSVKRKCNDYFRSIASWSCEAASQRAQSNLKNAFPYQKALVKCYKNLDYKGVKKNDMWYRLCSNRIFRYSRIAEDIAQYQSDKGKAKFEFVILAQSVAEYDISAIM

SEQ ID NO: 118 - protein, 602 aa, synthetic construct
  REGION 1..602: note = Description of Artificial Sequence: Synthetic polypeptide
VFLTDDKRKTALRKIRSAFRKTAEIALVRAQEADSLDRQAKKLTIETVSFGAPGAKNAFIGSLQGYNWNSHRANVPSSGSAKDVFRITELGLGIPQSAHEASIGKSFELVGNVVRYTANLLSKGYKKGAVNKGAKQQREIKGKEQLSFDLISNGPISGDKLINGQKDALAWWLIDKMGFHIGLAMEPLSSPNTYGITLQAFWKRHTAPRRYSRGVIRQWQLPFGRQLAPLIHNFFRKKGASIPIVLTNASKKLAGKGVLLEQTALVDPKKWWQVKEQVTGPLSNIWERSVPLVLYTATFTHKHGAAHKRPLTLKVIRISSGSVFLLPLSKVTPGKLVRAWMPDINILRDGRPDEAAYKGPDLIRARERSFPLAYTCVTQIADEWQKRALESNRDSITPLEAKLVTGSDLLQIHSTVQQAVEQGIGGRISSPIQELLAKDALQLVLQQLFMTVDLLRIQWQLKQEVADGNTSEKAVGWAIRISNIHKDAYKTAIEPCTSALKQAWNPLSGFEERTFQLDASIVRKRSTAKTPDDELVIVLRQQAAEMTVAVTQSVSKELMELAVRHSATLHLLVGEVASKQLSRSADKDRGAMDHWKLLSQSM

SEQ ID NO: 119 - protein, 494 aa, synthetic construct
  REGION 1..494: note = Description of Artificial Sequence: Synthetic polypeptide
EDLLQKALNTATNVAAIERHSCISCLFTESEIDVKYKTPDKIGQNTAGCQSCTFRVGYSGNSHTLPMGNRIALDKLRETIQRYAWHSLLFNVPPAPTSKRVRAISELRVAAGRERLFTVITFVQTNILSKLQKRYAANWTPKSQERLSRLREEGQHILSLLESGSWQQKEVVREDQDLIVCSALTKPGLSIGAFCRPKYLKPAKHALVLRLIFVEQWPGQIWGQSKRTRRMRRRKDVERVYDISVQAWALKGKETRISECIDTMRRHQQAYIGVLPFLILSGSTVRGKGDCPILKEITRMRYCPNNEGLIPLGIFYRGSANKLLRVVKGSSFTLPMWQNIETLPHPEPFSPEGWTATGALYEKNLAYWSALNEAVDWYTGQILSSGLQYPNQNEFLARLQNVIDSIPRKWFRPQGLKNLKPNGQEDIVPNEFVIPQNAIRAHHVIEWYHKTNDLVAKTLLGWGSQTTLNQTRPQGDLRFTYTRYYFREKEVPEV

SEQ ID NO: 120 - protein, 649 aa, synthetic construct
  REGION 1..649: note = Description of Artificial Sequence: Synthetic polypeptide
VPKKKLMRELAKKAVFEAIFNDPIPGSFGCKRCTLIDGARVTDAIEKKQGAKRCAGCEPCTFHTLYDSVKHALPAATGCDRTAIDTGLWEILTALRSYNWMSFRRNAVSDASQKQVWSIEELAIWADKERALRVILSALTHTIGKLKNGFSRDGVWKGGKQLYENLAQKDLAKGLFANGEIFGKELVEADHDMLAWTIVPNHQFHIGLIRGNWKPAAVEASTAFDARWLTNGAPLRDTRTHGHRGRRFNRTEKLTVLCIKRDGGVSEEFRQERDYELSVMLLQPKNKLKPEPKGELNSFEDLHDHWWFLKGDEATALVGLTSDPTVGDFIQLGLYIRNPIKAHGETKRRLLICFEPPIKLPLRRAFPSEAFKTWEPTINVFRNGRRDTEAYYDIDRARVFEFPETRVSLEHLSKQWEVLRLEPDRENTDPYEAQQNEGAELQVYSLLQEAAQKMAPKVVIDPFGQFPLELFSTFVAQLFNAPLSDTKAKIGKPLDSGFVVESHLHLLEEDFAYRDFVRVTFMGTEPTFRVIHYSNGEGYWKKTVLKGKNNIRTALIPEGAKAAVDAYKNKRCPLTLEAAILNEEKDRRLVLGNKALSLLAQTARGNLTILEALAAEVLRPLSGTEGVVHLHACVTRHSTLTESTETDNM

SEQ ID NO: 121 - protein, 414 aa, synthetic construct
  REGION 1..414: note = Description of Artificial Sequence: Synthetic polypeptide
VEKLFSERLKRAMWLKNEAGRAPPAETLTLKHKRVSGGHEKVKEELQRVLRSLSGTNQAAWNLGLSGGREPKSSDALKGEKSRVVLETVVFHSGHNRVLYDVIEREDQVHQRSSIMHMRRKGSNLLRLWGRSGKVRRKMREEVAEIKPVWHKDSRWLAIVEEGRQSVVGISSAGLAVFAVQESQCTTAEPKPLEYVVSIWFRGSKALNPQDRYLEFKKLKTTEALRGQQYDPIPFSLKRGAGCSLAIRGEGIKFGSRGPIKQFFGSDRSRPSHADYDGKRRLSLFSKYAGDLADLTEEQWNRTVSAFAEDEVRRATLANIQDFLSISHEKYAERLKKRIESIEEPVSASKLEAYLSAIFETFVQQREALASNFLMRLVESVALLISLEEKSPRVEFRVARYLAESKEGFNRKAM

SEQ ID NO: 122 - protein, 413 aa, synthetic construct
  REGION 1..413: note = Description of Artificial Sequence: Synthetic polypeptide
VVITQSELYKERLLRVMEIKNDRGRKEPRESQGLVLRFTQVTGGQEKVKQKLWLIFEGFSGTNQASWNFGQPAGGRKPNSGDALKGPKSRVTYETVVFHFGLRLLSAVIERHNLKQQRQTMAYMKRRAAARKKWARSGKKCSRMRNEVEKIKPKWHKDPRWFDIVKEGEPSIVGISSAGFAIYIVEEPNFPRQDPLEIEYAISIWFRRDRSQYLTFKKIQKAEKLKELQYNPIPFRLKQEKTSLVFESGDIKFGSRGSIEHFRDEARGKPPKADMDNNRRLTMFSVFSGNLTNLTEEQYARPVSGLLAPDEKRMPTLLKKLQDFFTPIHEKYGERIKQRLANSEASKRPFKKLEEYLPAIYLEFRARREGLASNWVLVLINSVRTLVRIKSEDPYIEFKVSQYLLEKEDNKAL

SEQ ID NO: 123 - protein, 449 aa, synthetic construct
  REGION 1..449: note = Description of Artificial Sequence: Synthetic polypeptide
KQDALFEERLKKAIFIKRQADPLQREELSLLPPNRKIVTGGHESAKDTLKQILRAINGTNQASWNPGTPSGKRDSKSADALAGPKSRVKLETVVFHVGHRLLKKVVEYQGHQKQQHGLKAFMRTCAAMRKKWKRSGKVVGELREQLANIQPKWHYDSRPLNLCFEGKPSVVGLRSAGIALYTIQKSVVPVKEPKPIEYAVSIWFRGPKAMDREDRCLEFKKLKIATELRKLQFEPIVSTLTQGIKGFSLYIQGNSVKFGSRGPIKYFSNESVRQRPPKADPDGNKRLALFSKFSGDLSDLTEEQWNRPILAFEGIIRRATLGNIQDYLTVGHEQFAISLEQLLSEKESVLQMSIEQQRLKKNLGKKAENEWVESFGAEQARKKAQGIREYISGFFQEYCSQREQWAENWVQQLNKSVRLFLTIQDSTPFIEFRVARYLPKGEKKKGKAM

SEQ ID NO: 124 - protein, 711 aa, synthetic construct
  REGION 1..711: note = Description of Artificial Sequence: Synthetic polypeptide
ANHAERHKRLRKEANRAANRNRPLVADCDTGDPLVGICRLLRRGDKMQPNKTGCRSCEQVEPELRDAILVSGPGRLDNYKYELFQRGRAMAVHRLLKRVPKLNRPKKAAGNDEKKAENKKSEIQKEKQKQRRMMPAVSMKQVSVADFKHVIENTVRHLFGDRRDREIAECAALRAASKYFLKSRRVRPRKLPKLANPDHGKELKGLRLREKRAKLKKEKEKQAELARSNQKGAVLHVATLKKDAPPMPYEKTQGRNDYTTFVISAAIKVGATRGTKPLLTPQPREWQCSLYWRDGQRWIRGGLLGLQAGIVLGPKLNRELLEAVLQRPIECRMSGCGNPLQVRGAAVDFFMTTNPFYVSGAAYAQKKFKPFGTKRASEDGAAAKAREKLMTQLAKVLDKVVTQAAHSPLDGIWETRPEAKLRAMIMALEHEWIFLRPGPCHNAAEEVIKCDCTGGHAILWALIDEARGALEHKEFYAVTRAHTHDCEKQKLGGRLAGFLDLLIAQDVPLDDAPAARKIKTLLEATPPAPCYKAATSIATCDCEGKFDKLWAIIDATRAGHGTEDLWARTLAYPQNVNCKCKAGKDLTHRLADFLGLLIKRDGPFRERPPHKVTGDRKLVFSGDKKCKGHQYVILAKAHNEEVVRAWISRWGLKSRTNKAGYAATELNLLLNWLSICRRRWMDMLTVQRDTPYIRMKTGRLVVDDKKERKAM

SEQ ID NO: 125 - protein, 574 aa, synthetic construct
  REGION 1..574: note = Description of Artificial Sequence: Synthetic polypeptide
AKQREALRVALERGIVRASNRTYTLVTNCTKGGPLPEQCRMIERGKARAMKWEPKLVGCGSCAAATVDLPAIEEYAQPGRLDVAKYKLTTQILAMATRRMMVRAAKLSRRKGQWPAKVQEEKEEPPEPKKMLKAVEMRPVAIVDFNRVIQTTIEHLWAERANADEAELKALKAAAAYFGPSLKIRARGPPKAAIGRELKKAHRKKAYAERKKARRKRAELARSQARGAAAHAAIRERDIPPMAYERTQGRNDVTTIPIAAAIKIAATRGARPLPAPKPMKWQCSLYWNEGQRWIRGGMLTAQAYAHAANIHRPMRCEMWGVGNPLKVRAFEGRVADPDGAKGRKAEFRLQTNAFYVSGAAYRNKKFKPFGTDRGGIGSARKKRERLMAQLAKILDKVVSQAAHSPLDDIWHTRPAQKLRAMIKQLEHEWMFLRPQAPTVEGTKPDVDVAGNMQRQIKALMAPDLPPIEKGSPAKRFTGDKRKKGERAVRVAEAHSDEVVTAWISRWGIQTRRNEGSYAAQELELLLNWLQICRRRWLDMTAAQRVSPYIRMKSGRMITDAADEGVAPIPLVENM

SEQ ID NO: 126 - protein, 743 aa, synthetic construct
  REGION 1..743: note = Description of Artificial Sequence: Synthetic polypeptide
KSISGRSIKHMACLKDMLKSEITEIEEKQKKESLRKWDYYSKFSDEILFRRNLNVSANHDANACYGCNPCAFLKEVYGFRIERRNNERIISYRRGLAGCKSCVQSTGYPPIEFVRRKFGADKAMEIVREVLHRRNWGALARNIGREKEADPILGELNELLLVDARPYFGNKSAANETNLAFNVITRAAKKFRDEGMYDIHKQLDIHSEEGKVPKGRKSRLIRIERKHKAIHGLDPGETWRYPHCGKGEKYGVWLNRSRLIHIKGNEYRCLTAFGTTGRRMSLDVACSVLGHPLVKKKRKKGKKTVDGTELWQIKKATETLPEDPIDCTFYLYAAKPTKDPFILKVGSLKAPRWKKLHKDFFEYSDTEKTQGQEKGKRVVRRGKVPRILSLRPDAKFKVSIWDDPYNGKNKEGTLLRMELSGLDGAKKPLILKRYGEPNTKPKNFVFWRPHITPHPLTFTPKHDFGDPNKKTKRRRVFNREYYGHLNDLAKMEPNAKFFEDREVSNKKNPKAKNIRIQAKESLPNIVAKNGRWAAFDPNDSLWKLYLHWRGRRKTIKGGISQEFQEFKERLDLYKKHEDESEWKEKEKLWENHEKEWKKTLEIHGSIAEVSQRCVMQSMMGPLDGLVQKKDYVHIGQSSLKAADDAWTFSANRYKKATGPKWGKISVSNLLYDANQANAELISQSISKYLSKQKDNQGCEGRKMKFLIKIIEPLRENFVKHTRWLHEMTQKDCEVRAQFSRVSM

SEQ ID NO: 127 - protein, 769 aa, synthetic construct
  REGION 1..769: note = Description of Artificial Sequence: Synthetic polypeptide
FPSDVGADALKHVRMLQPRLTDEVRKVALTRAPSDRPALARFAAVAQDGLAFVRHLNVSANHDSNCTFPRDPRDPRRGPCEPNPCAFLREVWGFRIVARGNERALSYRRGLAGCKSCVQSTGFPSVPFHRIGADDCMRKLHEILKARNWRLLARNIGREREADPLLTELSEYLLVDARTYPDGAAPNSGRLAENVIKRAAKKFRDEGMRDIHAQLRVHSREGKVPKGRLQRLRRIERKHRAIHALDPGPSWEAEGSARAEVQGVAVYRSQLLRVGHHTQQIEPVGIVARTLFGVGRTDLDVAVSVLGAPLTKRKKGSKTLESTEDFRIAKARETRAEDKIEVAFVLYPTASLLRDEIPKDAFPAMRIDRFLLKVGSVQADREILLQDDYYRFGDAEVKAGKNKGRTVTRPVKVPRLQALRPDAKFRVNVWADPFGAGDSPGTLLRLEVSGVTRRSQPLRLLRYGQPSTQPANFLCWRPHRVPDPMTFTPRQKFGERRKNRRTRRPRVFERLYQVHIKHLAHLEPNRKWFEEARVSAQKWAKARAIRRKGAEDIPVVAPPAKRRWAALQPNAELWDLYAHDREARKRFRGGRAAEGEEFKPRLNLYLAHEPEAEWESKRDRWERYEKKWTAVLEEHSRMCAVADRTLPQFLSDPLGARMDDKDYAFVGKSALAVAEAFVEEGTVERAQGNCSITAKKKFASNASRKRLSVANLLDVSDKADRALVFQAVRQYVQRQAENGGVEGRRMAFLRKLLAPLRQNFVCHTRWLHM

SEQ ID NO: 128 - protein, 564 aa, synthetic construct
  REGION 1..564: note = Description of Artificial Sequence: Synthetic polypeptide
AARKKKRGKIGITVKAKEKSPPAAGPFMARKLVNVAANVDGVEVHLCVECEADAHGSASARLLGGCRSCTGSIGAEGRLMGSVDVDRERVIAEPVHTETERLGPDVKAFEAGTAESKYAIQRGLEYWGVDLISRNRARTVRKMEEADRPESSTMEKTSWDEIAIKTYSQAYHASENHLFWERQRRVRQHALALFRRARERNRGESPLQSTQRPAPLVLAALHAEAAAISGRARAEYVLRGPSANVRAAAADIDAKPLGHYKTPSPKVARGFPVKRDLLRARHRIVGLSRAYFKPSDVVRGTSDAIAHVAGRNIGVAGGKPKEIEKTFTLPFVAYWEDVDRVVHCSSFKADGPWVRDQRIKIRGVSSAVGTFSLYGLDVAWSKPTSFYIRCSDIRKKFHPKGFGPMKHWRQWAKELDRLTEQRASCVVRALQDDEELLQTMERGQRYYDVFSCAATHATRGEADPSGGCSRCELVSCGVAHKVTKKAKGDTGIEAVAVAGCSLCESKLVGPSKPRVHRQMAALRQSHALNYLRRLQREWEALEAVQAPTPYLRFKYARHLEVRSM

SEQ ID NO: 129 - protein, 565 aa, synthetic construct
  REGION 1..565: note = Description of Artificial Sequence: Synthetic polypeptide
AAKKKKQRGKIGISVKPKEGSAPPADGPFMARKLVNVAANVDGVEVNLCIECEADAHGSAPARLLGGCKSCTGSIGAEGRLMGSVDVDRADAIAKPVNTETEKLGPDVQAFEAGTAETKYALQRGLEYWGVDLISRNRSRTVRRTEEGQPESATMEKTSWDEIAIKSYTRAYHASENHLFWERQRRVRQHALALFKRAKERNRGDSTLPREPGHGLVAIAALACEAYAVGGRNLAETVVRGPTFGTARAVRDVEIASLGRYKTPSPKVAHGSPVKRDFLRARHRIVGLARAYYRPSDVVRGTSDAIAHVAGRNIGVAGGKPRAVEAVFTLPFVAYWEDVDRVVHCSSFQVSAPWNRDQRMKIAGVTTAAGTFSLHGGELKWAKPTSFYIRCSDTRRKFRPKGFGPMKRWRQWAKDLDRLVEQRASCVVRALQDDAALLETMERGQRYYDVFACAVTHATRGEADRLAGCSRCALTPCQEAHRVTTKPRGDAGVEQVQTSDCSLCEGKLVGPSKPRLHRTLTLLRQEHGLNYLRRLQREWESLEAVQVPTPYLRFKYARHLEVRSM

SEQ ID NO: 130 - protein, 499 aa, synthetic construct
  REGION 1..499: note = Description of Artificial Sequence: Synthetic polypeptide
TDSQSESVPEVVYALTGGEVPGRVPPDGGSAEGARNAPTGLRKQRGKIKISAKPSKPGSPASSLARTLVNEAANVDGVQSSGCATCRMRANGSAPRALPIGCVACASSIGRAPQEETVCALPTTQGPDVRLLEGGHALRKYDIQRALEYWGVDLIGRNLDRQAGRGMEPAEGATATMKRVSMDELAVLDFGKSYYASEQHLFAARQRRVRQHAKALKIRAKHANRSGSVKRALDRSRKQVTALAREFFKPSDVVRGDSDALAHVVGRNLGVSRHPAREIPQTFTLPLCAYWEDVDRVISCSSLLAGEPFARDQEIRIEGVSSALGSLRLYRGAIEWHKPTSLYIRCSDTRRKFRPRGGLKKRWRQWAKDLDRLVEQRACCIVRSLQADVELLQTMERAQRFYDVHDCAATHVGPVAVRCSPCAGKQFDWDRYRLLAALRQEHALNYLRRLQREWESLEAQQVKMPYLRFKYARKLEVSGPLIGLEVRREPSMGTAIAEM

SEQ ID NO: 131 - protein, 358 aa, synthetic construct
  REGION 1..358: note = Description of Artificial Sequence: Synthetic polypeptide
AGTAGRRHGSLGARRSINIAGVTDRHGRWGCESCVYTRDQAGNRARCAPCDQSTYAPDVQEVTIGQRQAKYTIFLTLQSFSWTNTMRNNKRAAAGRSKRTTGKRIGQLAEIKITGVGLAHAHNVIQRSLQHNITKMWRAEKGKSKRVARLKKAKQLTKRRAYFRRRMSRQSRGNGFFRTGKGGIHAVAPVKIGLDVGMIASGSSEPADEQTVTLDAIWKGRKKKIRLIGAKGELAVAACRFREQQTKGDKCIPLILQDGEVRWNQNNWQCHPKKLVPLCGLEVSRKFVSQADRLAQNKVASPLAARFDKTSVKGTLVESDFAAVLVNVTSIYQQCHAMLLRSQEPTPSLRVQRTITSM

SEQ ID NO: 132 - protein, 369 aa, synthetic construct
  REGION 1..369: note = Description of Artificial Sequence: Synthetic polypeptide
GVRFSPAQSQVFFRTVIPQSVEARFAINMAAIHDAAGAFGCSVCRFEDRTPRNAKAVHGCSPCTRSTNRPDVFVLPVGAIKAKYDVFMRLLGFNWTHLNRRQAKRVTVRDRIGQLDELAISMLTGKAKAVLKKSICHNVDKSFKAMRGSLKKLHRKASKTGKSQLRAKLSDLRERTNTTQEGSHVEGDSDVALNKIGLDVGLVGKPDYPSEESVEVVVCLYFVGKVLILDAQGRIRDMRAKQYDGFKIPIIQRGQLTVLSVKDLGKWSLVRQDYVLAGDLRFEPKISKDRKYAECVKRIALITLQASLGFKERIPYYVTKQVEIKNASHIAFVTEAIQNCAENFREMTEYLMKYQEKSPDLKVLLTQLM

SEQ ID NO: 133 - protein, 486 aa, synthetic construct
  REGION 1..486: note = Description of Artificial Sequence: Synthetic polypeptide
RAVVGKVFLEQARRALNLATNFGTNHRTGCNGCYVTPGKLSIPQDGEKNAAGCTSCLMKATASYVSYPKPLGEKVAKYSTLDALKGFPWYSLRLNLRPNYRGKPINGVQEVAPVSKFRLAEEVIQAVQRYHFTELEQSFPGGRRRLRELRAFYTKEYRRAPEQRQHVVNGDRNIVVVTVLHELGFSVGMFNEVELLPKTPIECAVNVFIRGNRVLLEVRKPQFDKERLLVESLWKKDSRRHTAKWTPPNNEGRIFTAEGWKDFQLPLLLGSTSRSLRAIEKEGFVQLAPGRDPDYNNTIDEQHSGRPFLPLYLYLQGTISQEYCVFAGTWVIPFQDGISPYSTKDTFQPDLKRKAYSLLLDAVKHRLGNKVASGLQYGRFPAIEELKRLVRMHGATRKIPRGEKDLLKKGDPDTPEWWLLEQYPEFWRLCDAAAKRVSQNVGLLLSLKKQPLWQRRWLESRTRNEPLDNLPLSMALTLHLTNEEAL

SEQ ID NO: 134 - protein, 400 aa, synthetic construct
  REGION 1..400: note = Description of Artificial Sequence: Synthetic polypeptide
AAVYSKFYIENHFKMGIPETLSRIRGPSIIQGFSVNENYINIAGVGDRDFIFGCKKCKYTRGKPSSKKINKCHPCKRSTYPEPVIDVRGSISEFKYKIYNKLKQEPNQSIKQNTKGRMNPSDHTSSNDGIIINGIDNRIAYNVIFSSYKHLMEKQINLLRDTTKRKARQIKKYNNSGKKKHSLRSQTKGNLKNRYHMLGMFKKGSLTITNEGDFITAVRKVGLDISLYKNESLNKQEVETELCLNIKWGRTKSYTVSGYIPLPINIDWKLYLFEKETGLTLRLFGNKYKIQSKKFLIAQLFKPKRPPCADPVVKKAQKWSALNAHVQQMAGLFSDSHLLKRELKNRMHKQLDFKSLWVGTEDYIKWFEELSRSYVEGAEKSLEFFRQDYFCFNYTKQTTM

SEQ ID NO: 135 - protein, 666 aa, synthetic construct
  REGION 1..666: note = Description of Artificial Sequence: Synthetic polypeptide
PQQQRDLMLMAANYDQDYGNGCGPCTVVASAAYRPDPQAQHGCKRHLRTLGASAVTHVGLGDRTATITALHRLRGPAALAARARAAQAASAPMTPDTDAPDDRRRLEAIDADDVVLVGAHRALWSAVRRWADDRRAALRRRLHSEREWLLKDQIRWAELYTLIEASGTPPQGRWRNTLGALRGQSRWRRVLAPTMRATCAETHAELWDALAELVPEMAKDRRGLLRPPVEADALWRAPMIVEGWRGGHSVVVDAVAPPLDLPQPCAWTAVRLSGDPRQRWGLHLAVPPLGQVQPPDPLKATLAVSMRHRGGVRVRTLQAMAVDADAPMQRHLQVPLTLQRGGGLQWGIHSRGVRRREARSMASWEGPPIWTGLQLVNRWKGQGSALLAPDRPPDTPPYAPDAAVAPAQPDTKRARRTLKEACTVCRCAPGHMRQLQVTLTGDGTWRRFRLRAPQGAKRKAEVLKVATQHDERIANYTAWYLKRPEHAAGCDTCDGDSRLDGACRGCRPLLVGDQCFRRYLDKIEADRDDGLAQIKPKAQEAVAAMAAKRDARAQKVAARAAKLSEATGQRTAATRDASHEARAQKELEAVATEGTTVRHDAAAVSAFGSWVARKGDEYRHQVGVLANRLEHGLRLQELMAPDSVVADQQRASGHARVGYRYVLTAM

SEQ ID NO: 136 - protein, 560 aa, synthetic construct
  REGION 1..560: note = Description of Artificial Sequence: Synthetic polypeptide
AVAHPVGRGNAGSPGARGPEELPRQLVNRASNVTRPATYGCAPCRHVRLSIPKPVLTGCRACEQTTHPAPKRAVRGGADAAKYDLAAFFAGWAADLEGRNRRRQVHAPLDPQPDPNHEPAVTLQKIDLAEVSIEEFQRVLARSVKHRHDGRASREREKARAYAQVAKKRRNSHAHGARTRRAVRRQTRAVRRAHRMGANSGEILVASGAEDPVPEAIDHAAQLRRRIRACARDLEGLRHLSRRYLKTLEKPCRRPRAPDLGRARCHALVESLQAAERELEELRRCDSPDTAMRRLDAVLAAAASTDATFATGWTVVGMDLGVAPRGSAAPEVSPMEMAISVFWRKGSRRVIVSKPIAGMPIRRHELIRLEGLGTLRLDGNHYTGAGVTKGRGLSEGTEPDFREKSPSTLGFTLSDYRHESRWRPYGAKQGKTARQFFAAMSRELRALVEHQVLAPMGPPLLEAHERRFETLLKGQDNKSIHAGGGGRYVWRGPPDSKKRPAADGDWFRFGRGHADHRGWANKRHELAANYLQSAFRLWSTLAEAQEPTPYARYKYTRVTM

SEQ ID NO: 137 - protein, 404 aa, synthetic construct
  REGION 1..404: note = Description of Artificial Sequence: Synthetic polypeptide
WDFLTLQVYERHTSPEVCVAGNSTKCASGTRKSDHTHGVGVKLGAQEINVSANDDRDHEVGCNICVISRVSLDIKGWRYGCESCVQSTPEWRSIVRFDRNHKEAKGECLSRFEYWGAQSIARSLKRNKLMGGVNLDELAIVQNENVVKTSLKHLFDKRKDRIQANLKAVKVRMRERRKSGRQRKALRRQCRKLKRYLRSYDPSDIKEGNSCSAFTKLGLDIGISPNKPPKIEPKVEVVFSLFYQGACDKIVTVSSPESPLPRSWKIKIDGIRALYVKSTKVKFGGRTFRAGQRNNRRKVRPPNVKKGKRKGSRSQFFNKFAVGLDAVSQQLPIASVQGLWGRAETKKAQTICLKQLESNKPLKESQRCLFLADNWVVRVCGFLRALSQRQGPTPYIRYRYRCNM

SEQ ID NO: 138 - protein, 392 aa, synthetic construct
  REGION 1..392: note = Description of Artificial Sequence: Synthetic polypeptide
ARNVGQRNASRQSKRESAKARSRRVTGGHASVTQGVALINAAANADRDHTTGCEPCTWERVNLPLQEVIHGCDSCTKSSPFWRDIKVVNKGYREAKEEIMRIASGISADHLSRALSHNKVMGRLNLDEVCILDFRTVLDTSLKHLTDSRSNGIKEHIRAVHRKIRMRRKSGKTARALRKQYFALRRQWKAGHKPNSIREGNSLTALRAVGFDVGVSEGTEPMPAPQTEVVLSVFYKGSATRILRISSPHPIAKRSWKVKIAGIKALKLIRREHDFSFGRETYNASQRAEKRKFSPHAARKDFFNSFAVQLDRLAQQLCVSSVENLWVTEPQQKLLTLAKDTAPYGIREGARFADTRARLAWNWVFRVCGFTRALHQEQEPTPYCRFTWRSKM

SEQ ID NO: 139 - protein, 707 aa, synthetic construct
  REGION 1..707: note = Description of Artificial Sequence: Synthetic polypeptide
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 140 - protein, 757 aa, synthetic construct
  REGION 1..757: note = Description of Artificial Sequence: Synthetic polypeptide
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 141 - protein, 765 aa, synthetic construct
  REGION 1..765: note = Description of Artificial Sequence: Synthetic polypeptide
MYILEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 142 - protein, 766 aa, synthetic construct
  REGION 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 143 - protein, 812 aa, synthetic construct
  REGION 1..812: note = Description of Artificial Sequence: Synthetic polypeptide
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 144 - protein, 812 aa, synthetic construct
  REGION 1..812: note = Description of Artificial Sequence: Synthetic polypeptide
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHKGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 145 - protein, 793 aa, synthetic construct
  REGION 1..793: note = Description of Artificial Sequence: Synthetic polypeptide
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 146 - protein, 441 aa, synthetic construct
  REGION 1..441: note = Description of Artificial Sequence: Synthetic polypeptide
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 147 - protein, 812 aa, synthetic construct
  REGION 1..812: note = Description of Artificial Sequence: Synthetic polypeptide
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 148 - protein, 812 aa, synthetic construct
  REGION 1..812: note = Description of Artificial Sequence: Synthetic polypeptide
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 149 - protein, 761 aa, synthetic construct
  REGION 1..761: note = Description of Artificial Sequence: Synthetic polypeptide
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 150 - protein, 717 aa, synthetic construct
  REGION 1..717: note = Description of Artificial Sequence: Synthetic polypeptide
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 151 - protein, 781 aa, synthetic construct
  REGION 1..781: note = Description of Artificial Sequence: Synthetic polypeptide
MRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 152 - protein, 793 aa, synthetic construct; identical to SEQ ID NO: 145
  REGION 1..793: note = Description of Artificial Sequence: Synthetic polypeptide

SEQ ID NO: 153 - protein, 717 aa, synthetic construct; identical to SEQ ID NO: 150
  REGION 1..717: note = Description of Artificial Sequence: Synthetic polypeptide

SEQ ID NO: 154 - protein, 761 aa, synthetic construct; identical to SEQ ID NO: 149
  REGION 1..761: note = Description of Artificial Sequence: Synthetic polypeptide

SEQ ID NO: 155 - protein, 765 aa, synthetic construct
  REGION 1..765: note = Description of Artificial Sequence: Synthetic polypeptide
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 156 - protein, 766 aa, synthetic construct
  REGION 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 157 - protein, 870 aa, synthetic construct
  REGION 1..870: note = Description of Artificial Sequence: Synthetic polypeptide
MLVRTSTLVQDNKNSRSASRAFLKKPKMPKNKHIKEPTELAKLIRELFPGQRFTRAINTQAGKILKHKGRDEVVEFLKNKGIDKEQFMDFRPPTKARIVATSGAIEEFSYLRVSMAIQECCFGKYKFPKEKVNGKLVLETVGLTKEELDDFLPKKYYENKKSRDRFFLKTGICDYGYTYAQGLNEIFRNTRAIYEGVFTKVNNRNEKRREKKDKYNEERRSKGLSEEPYDEDESATDESGHLINPPGVNLNIWTCEGFCKGPYVTKLSGTPGYEVILPKVFDGYNRDPNEIISCGITDRFAIPEGEPGHIPWHQRLEIPEGQPGYVPGHQRFADTGQNNSGKANPNKKGRMRKYYGHGTKYTQPGEYQEVFRKGHREGNKRRYWEEDFRSEAHDCILYVIHIGDDWVVCDLRGPLRDAYRRGLVPKEGITTQELCNLFSGDPVIDPKHGVVTFCYKNGLVRAQKTISAGKKSRELLGALTSQGPIALIGVDLGQTEPVGARAFIVNQARGSLSLPTLKGSFLLTAENSSSWNVFKGEIKAYREAIDDLAIRLKKEAVATLSVEQQTEIESYEAFSAEDAKQLACEKFGVDSSFILWEDMTPYHTGPATYYFAKQFLKKNGGNKSLIEYIPYQKKKSKKTPKAVLRSDYNIACCVRPKLLPETRKALNEAIRIVQKNSDEYQRLSKRKLEFCRRVVNYLVRKAKKLTGLERVIIAIEDLKSLEKFFTGSGKRDNGWSNFFRPKKENRWFIPAFHKAFSELAPNRGFYVIECNPARTSITDPDCGYCDGDNRDGIKFECKKCGAKHHTDLDVAPLNIAIVAVTGRPMPKTVSNKSKRERSGGEKSVGASRKRNHRKSKANQEMLDATSSAAE

SEQ ID NO: 158 - protein, 830 aa, synthetic construct
  REGION 1..830: note = Description of Artificial Sequence: Synthetic polypeptide
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 159 - protein, 790 aa, synthetic construct
  REGION 1..790: note = Description of Artificial Sequence: Synthetic polypeptide
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLNADLDVATTNLVRVALTGKVMPRSERSGDAQTPGPARKARTGKIKGSKPTSAPQGATQTDAKAHLSQTGV

SEQ ID NO: 160 - protein, 790 aa, synthetic construct
  REGION 1..790: note = Description of Artificial Sequence: Synthetic polypeptide
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLHADLDVATTNLVRVALTGKVMPRSERSGDAQTPGPARKARTGKIKGSKPTSAPQGATQTDAKAHLSQTGV

SEQ ID NO: 161 - protein, 782 aa, synthetic construct
  REGION 1..782: note = Description of Artificial Sequence: Synthetic polypeptide
MKTEKPKTALTLLREEVFPGKKYRLDVLKEAGKKLSTKGREATIEFLTGKDEERPQNFQPPAKTSIVAQSRPFDQWPIVQVSLAVQKYIYGLTQSEFEANKKALYGETGKAISTESRRAWFEATGVDNFGFTAAQGINPIFSQAVARYEGVIKKVENRNEKKLKKLTKKNLLRLESGEEIEDFEPEATFNEEGRLLQPPGANPNIYCYQQISPRIYDPSDPKGVILPQIYAGYDRKPEDIISAGVPNRLAIPEGQPGYIPEHQRAGLKTQGRIRCRASVEAKARAAILAVVHLGEDWVVLDLRGLLRNVYWRKLASPGTLTLKGLLDFFTGGPVLDARRGIATFSYTLKSAAAVHAENTYKGKGTREVLLKLTENNSVALVTVDLGQRNPLAAMIARVSRTSQGDLTYPESVEPLTRLFLPDPFLEEVRKYRSSYDALRLSIREAAIASLTPEQQAEIRYIEKFSAGDAKKNVAEVFGIDPTQLPWDAMTPRTTYISDLFLRMGGDRSRVFFEVPPKKAKKAPKKPPKKPAGPRIVKRTDGMIARLREIRPRLSAETNKAFQEARWEGERSNVAFQKLSVRRKQFARTVVNHLVQTAQKMSRCDTVVLGIEDLNVPFFHGRGKYQPGWEGFFRQKKENRWLINDMHKALSERGPHRGGYVLELTPFWTSLRCPKCGHTDSANRDGDDFVCVKCGAKLHSDLEVATANLALVAITGQSIPRPPREQSSGKKSTGTARMKKTSGETQGKGSKACVSEALNKIEQGTARDPVYNPLNSQVSCPAP

SEQ ID NO: 162 - protein, 774 aa, synthetic construct
  REGION 1..774: note = Description of Artificial Sequence: Synthetic polypeptide
VYNPDMKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 163 - protein, 769 aa, synthetic construct
  REGION 1..769: note = Description of Artificial Sequence: Synthetic polypeptide
MKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 164 - protein, 767 aa, synthetic construct
  REGION 1..767: note = Description of Artificial Sequence: Synthetic polypeptide
VIKTHFPAGRFRKDHQKTAGKKLKHEGEEACVEYLRNKVSDYPPNFKPPAKGTIVAQSRPFSEWPIVRASEAIQKYVYGLTVAELDVFSPGTSKPSHAEWFAKTGVENYGYRQVQGLNTIFQNTVNRFKGVLKKVENRNKKSLKRQEGANRRRVEEGLPEVPVTVESATDDEGRLLQPPGVNPSIYGYQGVAPRVCTDLQGFSGMSVDFAGYRRDPDAVLVESLPEGRLSIPKGERGYVPEWQRDPERNKFPLREGSRRQRKWYSNACHKPKPGRTSKYDPEALKKASAKDALLVSISIGEDWAIIDVRGLLRDARRRGFTPEEGLSLNSLLGLFTEYPVFDVQRGLITFTYKLGQVDVHSRKTVPTFRSRALLESLVAKEEIALVSVDLGQTNPASMKVSRVRAQEGALVAEPVHRMFLSDVLLGELSSYRKRMDAFEDAIRAQAFETMTPEQQAEITRVCDVSVEVARRRVCEKYSISPQDVPWGEMTGHSTFIVDAVLRKGGDESLVYFKNKEGETLKFRDLRISRMEGVRPRLTKDTRDALNKAVLDLKRAHPTFAKLAKQKLELARRCVNFIEREAKRYTQCERVVFVIEDLNVGFFHGKGKRDRGWDAFFTAKKENRWVIQALHKAFSDLGLHRGSYVIEVTPQRTSMTCPRCGHCDKGNRNGEKFVCLQCGATLHADLEVATDNIERVALTGKAMPKPPVRERSGDVQKAGTARKARKPLKPKQKTEPSVQEGSSDDGVDKSPGDASRNPVYNPSDTLSI

SEQ ID NO: 165 - protein, 763 aa, synthetic construct
  REGION 1..763: note = Description of Artificial Sequence: Synthetic polypeptide
MAKAKTLAALLRELLPGQHLAPHHRWVANKLLMTSGDAAAFVIGKSVSDPVRGSFRKDVITKAGRIFKKDGPDAAAAFLDGKWEDRPPNFQPPAKAAIVAISRSFDEWPIVKVSCAIQQYLYALPVQEFESSVPEARAQAHAAWFQDTGVDDCNFKSTQGLNAIFNHGKRTYEGVLKKAQNRNDKKNLRLERINAKRAEAGQAPLVAGPDESPTDDAGCLLHPPGINANIYCYQQVSPRPYEQSCGIQLPPEYAGYNRLSNVAIPPMPNRLDIPQGQPGYVPEHHRHGIKKFGRVRKRYGVVPGRNRDADGKRTRQVLTEAGAAAKARDSVLAVIRIGDDWTVVDLRGLLRNAQWRKLVPDGGITVQGLLDLFTGDPVIDPRRGVVTFIYKADSVGIHSEKVCRGKQSKNLLERLCAMPEKSSTRLDCARQAVALVSVDLGQRNPVAARFSRVSLAEGQLQAQLVSAQFLDDAMVAMIRSYREEYDRFESLVREQAKAALSPEQLSEIVRHEADSAESVKSCVCAKFGIDPAGLSWDKMTSGTWRIADHVQAAGGDVEWFFFKTCGKGKEIKTVRRSDFNVAKQFRLRLSPETRKDWNDAIWELKRGNPAYVSFSKRKSEFARRVVNDLVHRARRAVRCDEVVFAIEDLNISFFHGKGQRQMGWDAFFEVKQENRWFIQALHKAFVERATHKGGYVLEVAPARTSTTCPECRHCDPESRRGEQFCCIKCRHTCHADLEVATFNIEQVALTGVSLPKRLSSTLL

SEQ ID NO: 166 - protein, 761 aa, synthetic construct
  REGION 1..761: note = Description of Artificial Sequence: Synthetic polypeptide
MSKEKTPPSAYAILKAKHFPDLDFEKKHKMMAGRMFKNGASEQEVVQYLQGKGSESLMDVKPPAKSPILAQSRPFDEWEMVRTSRLIQETIFGIPKRGSIPKRDGLSETQFNELVASLEVGGKPMLNKQTRAIFYGLLGIKPPTFHAMAQNILIDLAINIRKGVLKKVDNLNEKNRKKVKRIRDAGEQDVMVPAEVTAHDDRGYLNHPPGVNPTIPGYQGVVIPFPEGFEGLPSGMTPVDWSHVLVDYLPHDRLSIPKGSPGYIPEWQRPLLNRHKGRRHRSWYANSLNKPRKSRTEEAKDRQNAGKRTALIEAERLKGVLPVLMRFKEDWLIIDARGLLRNARYRGVLPEGSTLGNLIDLFSDSPRVDTRRGICTFLYRKGRAYSTKPVKRKESKETLLKLTEKSTIALVSIDLGQTNPLTAKLSKVRQVDGCLVAEPVLRKLIDNASEDGKEIARYRVAHDLLRARILEDAIDLLGIYKDEVVRARSDTPDLCKERVCRFLGLDSQAIDWDRMTPYTDFIAQAFVAKGGDPKVVTIKPNGKPKMFRKDRSIKNMKGIRLDISKEASSAYREAQWAIQRESPDFQRLAVWQSQLTKRIVNQLVAWAKKCTQCDTVVLAFEDLNIGMMHGSGKWANGGWNALFLHKQENRWFMQAFHKALTELSAHKGIPTIEVLPHRTSITCTQCGHCHPGNRDGERFKCLKCEFLANTDLEIATDNIERVALTGLPMPKGERSSAKRKPGGTRKTKKSKHSGNSPLAAE

SEQ ID NO: 167 - protein, 746 aa, synthetic construct
  REGION 1..746: note = Description of Artificial Sequence: Synthetic polypeptide
MEKAGPTSPLSVLIHKNFEGCRFQIDHLKIAGRKLAREGEAAAIEYLLDKKCEGLPPNFQPPAKGNVIAQSRPFTEWAPYRASVAIQKYIYSLSVDERKVCDPGSSSDSHEKWFKQTGVQNYGYTHVQGLNLIFKHALARYDGVLKKVDNRNEKNRKKAERVNSFRREEGLPEEVFEEEKATDETGHLLQPPGVNHSIYCYQSVRPKPFNPRKPGGISLPEAYSGYSLKPQDELPIGSLDRLSIPPGQPGYVPEWQRSQLTTQKHRRKRSWYSAQKWKPRTGRTSTFDPDRLNCARAQGAILAVVRIHEDWVVFDVRGLLRNALWRELAGKGLTVRDLLDFFTGDPVVDTKRGVVTFTYKLGKVDVHSLRTVRGKRSKKVLEDLTLSSDVGLVTIDLGQTNVLAADYSKVTRSENGELLAVPLSKSFLPKHLLHEVTAYRTSYDQMEEGFRRKALLTLTEDQQVEVTLVRDFSVESSKTKLLQLGVDVTSLPWEKMSSNTTYISDQLLQQGADPASLFFDGERDGKPCRHKKKDRTWAYLVRPKVSPETRKALNEALWALKNTSPEFESLSKRKIQFSRRCMNYLLNEAKRISGCGQVVFVIEDLNVRVHHGRGKRAIGWDNFFKPKRENRWFMQALHKAASELAIHRGMHIIEACPARSSITCPKCGHCDPENRCSSDREKFLCVKCGAAFHADLEVATFNLRKVALTGTALPKSIDHSRDGLIPKGARNRKLKEPQANDEKACA

SEQ ID NO: 168 - protein, 735 aa, synthetic construct
  REGION 1..735: note = Description of Artificial Sequence: Synthetic polypeptide
MKEQSPLSSVLKSNFPGKKFLSADIRVAGRKLAQLGEAAAVEYLSPRQRDSVPNFRPPAFCTVVAKSRPFEEWPIYKASVLLQEQIYGMTGQEFEERCGSIPTSLSGLRQWASSVGLGAAMEGLHVQGMNLMVKNAINRYKGVLVKVENRNKKLVEANEAKNSSREERGLPPLRPPELGSAFGPDGRLVNPPGIDKSIRLYQGVSPVPVVKTTGRPTVHRLDIPAGEKGHVPLWQREAGLVKEGPRRRRMWYSNSNLKRSRKDRSAEASEARKADSVVVRVSVKEDWVDIDVRGLLRNVAWRGIERAGESTEDLLSLFSGDPVVDPSRDSVVFLYKEGVVDVLSKKVVGAGKSRKQLEKMVSEGPVALVSCDLGQTNYVAARVSVLDESLSPVRSFRVDPREFPSADGSQGVVGSLDRIRADSDRLEAKLLSEAEASLPEPVRAEIEFLRSERPSAVAGRLCLKLGIDPRSIPWEKMGSTTSFISEALSAKGSPLALHDGAPIKDSRFAHAARGRLSPESRKALNEALWERKSSSREYGVISRRKSEASRRMANAVLSESRRLTGLAVVAVNLEDLNMVSKFFHGRGKRAPGWAGFFTPKMENRWFIRSIHKAMCDLSKHRGITVIESRPERTSISCPECGHCDPENRSGERFSCKSCGVSLHADFEVATRNLERVALTGKPMPRRENLHSPEGATASRKTRKKPREATASTFLDLRSVLSSAENEGSGPAARAG

SEQ ID NO: 169 - protein, 725 aa, synthetic construct
  REGION 1..725: note = Description of Artificial Sequence: Synthetic polypeptide
MLPPSNKIGKSMSLKEFINKRNFKSSIIKQAGKILKKEGEEAVKKYLDDNYVEGYKKRDFPITAKCNIVASNRKIEDFDISKFSSFIQNYVFNLNKDNFEEFSKIKYNRKSFDELYKKIANEIGLEKPNYENIQGEIAVIRNAINIYNGVLKKVENRNKKIQEKNQSKDPPKLLSAFDDNGFLAERPGINETIYGYQSVRLRHLDVEKDKDIIVQLPDIYQKYNKKSTDKISVKKRLNKYNVDEYGKLISKRRKERINKDDAILCVSNFGDDWIIFDARGLLRQTYRYKLKKKGLCIKDLLNLFTGDPIINPTKTDLKEALSLSFKDGIINNRTLKVKNYKKCPELISELIRDKGKVAMISIDLGQTNPISYRLSKFTANNVAYIENGVISEDDIVKMKKWREKSDKLENLIKEEAIASLSDDEQREVRLYENDIADNTKKKILEKFNIREEDLDFSKMSNNTYFIRDCLKNKNIDESEFTFEKNGKKLDPTDACFAREYKNKLSELTRKKINEKIWEIKKNSKEYHKISIYKKETIRYIVNKLIKQSKEKSECDDIIVNIEKLQIGGNFFGGRGKRDPGWNNFFLPKEENRWFINACHKAFSELAPHKGIIVIESDPAYTSQTCPKCENCDKENRNGEKFKCKKCNYEANADIDVATENLEKIAKNGRRLIKNFDQLGERLPGAEMPGGARKRKPSKSLPKNGRGAGVGSEPELINQSPSQVIA

SEQ ID NO: 170 - protein, 718 aa, synthetic construct
  REGION 1..718: note = Description of Artificial Sequence: Synthetic polypeptide
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 171 - protein, 708 aa, synthetic construct
  REGION 1..708: note = Description of Artificial Sequence: Synthetic polypeptide
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 172 - protein, 491 aa, synthetic construct
  REGION 1..491: note = Description of Artificial Sequence: Synthetic polypeptide
VLLSDRIQYTDPSAPIPAMTVVDRRKIKKGEPGYVPPFMRKNLSTNKHRRMRLSRGQKEACALPVGLRLPDGKDGWDFIIFDGRALLRACRRLRLEVTSMDDVLDKFTGDPRIQLSPAGETIVTCMLKPQHTGVIQQKLITGKMKDRLVQLTAEAPIAMLTVDLGEHNLVACGAYTVGQRRGKLQSERLEAFLLPEKVLADFEGYRRDSDEHSETLRHEALKALSKRQQREVLDMLRTGADQARESLCYKYGLDLQALPWDKMSSNSTFIAQHLMSLGFGESATHVRYRPKRKASERTILKYDSRFAAEEKIKLTDETRRAWNEAIWECQRASQEFRCLSVRKLQLARAAVNWTLTQAKQRSRCPRVVVVVEDLNVRFMHGGGKRQEGWAGFFKARSEKRWFIQALHKAYTELPTNRGIHVMEVNPARTSITCTKCGYCDPENRYGEDFHCRNPKCKVRGGHVANADLDIATENLARVALSGPMPKAPKLK

SEQ ID NO: 173 - protein, 848 aa, synthetic construct
  REGION 1..848: note = Description of Artificial Sequence: Synthetic polypeptide
MTPSFGYQMIIVTPIHHASGAWATLRLLFLNPKTSGVMLGMTKTKSAFALMREEVFPGLLFKSADLKMAGRKFAKEGREAAIEYLRGKDEERPANFKPPAKGDIIAQSRPFDQWPIVQVSQAIQKYIFGLTKAEFDATKTLLYGEGNHPTTESRRRWFEATGVPDFGFTSAQGLNAIFSSALARYEGVIQKVENRNEKRLKKLSEKNQRLVEEGHAVEAYVPETAFHTLESLKALSEKSLVPLDDLMDKIDRLAQPPGINPCLYGYQQVAPYIYDPENPRGVVLPDLYLGYCRKPDDPITACPNRLDIPKGQPGYIPEHQRGQLKKHGRVRRFRYTNPQAKARAKAQTAILAVLRIDEDWVVMDLRGLLRNVYFREVAAPGELTARTLLDTFTGCPVLNLRSNVVTFCYDIESKGALHAEYVRKGWATRNKLLDLTKDGQSVALLSVDLGQRHPVAVMISRLKRDDKGDLSEKSIQVVSRTFADQYVDKLKRYRVQYDALRKEIYDAALVSLPPEQQAEIRAYEAFAPGDAKANVLSVMFQGEVSPDELPWDKMNTNTHYISDLYLRRGGDPSRVFFVPQPSTPKKNAKKPPAPRKPVKRTDENVSHMPEFRPHLSNETREAFQKAKWTMERGNVRYAQLSRFLNQIVREANNWLVSEAKKLTQCQTVVWAIEDLHVPFFHGKGKYHETWDGFFRQKKEDRWFVNVFHKAISERAPNKGEYVMEVAPYRTSQRCPVCGFVDADNRHGDHFKCLRCGVELHADLEVATWNIALVAVQGHGIAGPPREQSCGGETAGTARKGKNIKKNKGLADAVTVEAQDSEGGSKKDAGTARNPVYIPSESQVNCPAP

SEQ ID NO: 174 - protein, 781 aa, synthetic construct
  REGION 1..781: note = Description of Artificial Sequence: Synthetic polypeptide
MKPKTPKPPKTPVAALIDKHFPGKRFRASYLKSVGKKLKNQGEDVAVRFLTGKDEERPPNFQPPAKSNIVAQSRPIEEWPIHKVSVAVQEYVYGLTVAEKEACSDAGESSSSHAAWFAKTGVENFGYTSVQGLNKIFPPTFNRFDGVIKKVENRNEKKRQKATRINEAKRNKGQSEDPPEAEVKATDDAGYLLQPPGINHSVYGYQSITLCPYTAEKFPTIKLPEEYAGYHSNPDAPIPAGVPDRLAIPEGQPGHVPEEHRAGLSTKKHRRVRQWYAMANWKPKPKRTSKPDYDRLAKARAQGALLIVIRIDEDWVVVDARGLLRNVRWRSLGKREITPNELLDLFTGDPVLDLKRGVVTFTYAEGVVNVCSRSTTKGKQTKVLLDAMTAPRDGKKRQIGMVAVDLGQTNPIAAEYSRVGKNAAGTLEATPLSRSTLPDELLREIALYRKAHDRLEAQLREEAVLKLTAEQQAENARYVETSEEGAKLALANLGVDTSTLPWDAMTGWSTCISDHLINHGGDTSAVFFQTIRKGTKKLETIKRKDSSWADIVRPRLTKETREALNDFLWELKRSHEGYEKLSKRLEELARRAVNHVVQEVKWLTQCQDIVIVIEDLNVRNFHGGGKRGGGWSNFFTVKKENRWFMQALHKAFSDLAAHRGIPVLEVYPARTSITCLGCGHCDPENRDGEAFVCQQCGATFHADLEVATRNIARVALTGEAMPKAPAREQPGGAKKRGTSRRRKLTEVAVKSAEPTIHQAKNQQLNGTSRDPVYKGSELPAL

SEQ ID NO: 175 - protein, 767 aa, synthetic construct
  REGION 1..767: note = Description of Artificial Sequence: Synthetic polypeptide
MSEITDLLKANFKGKTFKSADMRMAGRILKKSGAQAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRGVQTTGTARNPGRSLKSNKNPSEPKRVLQSKTRKKITSTETQNEPLATDLKT

SEQ ID NO: 176 - protein, 760 aa, synthetic construct
  REGION 1..760: note = Description of Artificial Sequence: Synthetic polypeptide
MTPKTESPLSALCKKHFPGKRFRTNYLKDAGKILKKHGEDAVVAFLSDKQEDEPANFCPPAKVHILAQSRPFEDWPINLASKAIQTYVYGLTADERKTCEPGTSKESHDRWFKETGVDHHGFTSVQGLNLIFKHTLNRYDGVIKKVETRNEKRRSSVVRINEKKAAEGLPLIAAEAEETAFGEDGRLLQPPGVNHSIYCFQQVSPQPYSSKKHPQVVLPHAVQGVDPDAPIPVGRPNRLDIPKGQPGYVPEWQRPHLSMKCKRVRMWYARANWRRKPGRRSVLNEARLKEASAKGALPIVLVIGDDWLVMDARGLLRSVFWRRVAKPGLSLSELLNVTPTGLFSGDPVIDPKRGLVTFTSKLGVVAVHSRKPTRGKKSKDLLLKMTKPTDDGMPRHVGMVAIDLGQTNPVAAEYSRVVQSDAGTLKQEPVSRGVLPDDLLKDVARYRRAYDLTEESIRQEAIALLSEGHRAEVTKLDQTTANETKRLLVDRGVSESLPWEKMSSNTTYISDCLVALGKTDDVFFVPKAKKGKKETGIAVKRKDHGWSKLLRPRTSPEARKALNENQWAVKRASPEYERLSRRKLELGRRCVNHIIQETKRWTQCEDIVVVLEDLNVGFFHGSGKRPDGWDNFFVSKRENRWFIQVLHKAFGDLATHRGTHVIEVHPARTSITCIKCGHCDAGNRDGESFVCLASACGDRRHADLEVATRNVARVAITGERMPPSEQARDVQKAGGARKRKPSARNVKSSYPAVEPAPASP

SEQ ID NO: 177 - protein, 752 aa, synthetic construct
  REGION 1..752: note = Description of Artificial Sequence: Synthetic polypeptide
MSDNKMKKLSKEEKPLTPLQILIRKYIDKSQYPSGFKTTIIKQAGVRIKSVKSEQDEINLANWIISKYDPTYIKRDFNPSAKCQIIATSRSVADFDIVKMSNKVQEIFFASSHLDKNVFDIGKSKSDHDSWFERNNVDRGIYTYSNVQGMNLIFSNTKNTYLGVAVKAQNKFSSKMKRIQDINNFRITNHQSPLPIPDEIKIYDDAGFLLNPPGVNPNIFGYQSCLLKPLENKEIISKTSFPEYSRLPADMIEVNYKISNRLKFSNDQKGFIQFKDKLNLFKINSQELFSKRRRLSGQPILLVASFGDDWVVLDGRGLLRQVYYRGIAKPGSITISELLGFFTGDPIVDPIRGVVSLGFKPGVLSQETLKTTSARIFAEKLPNLVLNNNVGLMSIDLGQTNPVSYRLSEITSNMSVEHICSDFLSQDQISSIEKAKTSLDNLEEEIAIKAVDHLSDEDKINFANFSKLNLPEDTRQSLFEKYPELIGSKLDFGSMGSGTSYIADELIKFENKDAFYPSGKKKFDLSFSRDLRKKLSDETRKSYNDALFLEKRTNDKYLKNAKRRKQIVRTVANSLVSKIEELGLTPVINIENLAMSGGFFDGRGKREKGWDNFFKVKKENRWVMKDFHKAFSELSPHHGVIVIESPPYCTSVTCTKCNFCDKKNRNGHKFTCQRCGLDANADLDIATENLEKVAISGKRMPGSERSSDERKVAVARKAKSPKGKAIKGVKCTITDEPALLSANSQDCSQSTS

SEQ ID NO: 178 - protein, 747 aa, synthetic construct
  REGION 1..747: note = Description of Artificial Sequence: Synthetic polypeptide
MALSLAEVRERHFKGLRFRSSYLKRAGKILKKEGEAACVAYLTGKDEESPPNFKPPAKCDVVAQSRPFEEWPIVQASVAVQSYVYGLTKEAFEAFNPGTTKQSHEACLAATGIDTCGYSNVQGLNLIFRQAKNRYEGVITKVENRNKKAKKKLTRKNEWRQKNGHSELPEAPEELTFNDEGRLLQPPGINPSLYTYQQISPTPWSPKDSSILPPQYAGYERDPNAPIPFGVAKDRLTIASGCPGYIPEWMRTAGEKTNPRTQKKFMHPGLSTRKNKRMRLPRSVRSAPLGALLVTIHLGEDWLVLDVRGLLRNARWRGVAPKDISTQGLLNLFTGDPVIDTRRGVVTFTYKPETVGIHSRTWLYKGKQTKEVLEKLTQDQTVALVAIDLGQTNPVSAAASRVSRSGENLSIETVDRFFLPDELIKELRLYRMAHDRLEERIREESTLALTEAQQAEVRALEHVVRDDAKNKVCAAFNLDAASLPWDQMTSNTTYLSEAILAQGVSRDQVFFTPNPKKGSKEPVEVMRKDRAWVYAFKAKLSEETRKAKNEALWALKRASPDYARLSKRREELCRRSVNMVINRAKKRTQCQVVIPVLEDLNIGFFHGSGKRLPGWDNFFVAKKENRWLMNGLHKSFSDLAVHRGFYVFEVMPHRTSITCPACGHCDSENRDGEAFVCLSCKRTYHADLDVATHNLTQVAGTGLPMPEREHPGGTKKPGGSRKPESPQTHAPILHRTDYSESADRLGS

SEQ ID NO: 179 - protein, 733 aa, synthetic construct
  REGION 1..733: note = Description of Artificial Sequence: Synthetic polypeptide
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 180 - protein, 702 aa, synthetic construct
  REGION 1..702: note = Description of Artificial Sequence: Synthetic polypeptide
MIKEQSELSKLIEKYYPGKKFYSNDLKQAGKHLKKSEHLTAKESEELTVEFLKSCKEKLYDFRPPAKALIISTSRPFEEWPIYKASESIQKYIYSLTKEELEKYNISTDKTSQENFFKESLIDNYGFANVSGLNLIFQHTKAIYDGVLKKVNNRNNKILKKYKRKIEEGIEIDSPELEKAIDESGHFINPPGINKNIYCYQQVSPTIFNSFKETKIICPFNYKRNPNDIIQKGVIDRLAIPFGEPGYIPDHQRDKVNKHKKRIRKYYKNNENKNKDAILAKINIGEDWVLFDLRGLLRNAYWRKLIPKQGITPQQLLDMFSGDPVIDPIKNNITFIYKESIIPIHSESIIKTKKSKELLEKLTKDEQIALVSIDLGQTNPVAARFSRLSSDLKPEHVSSSFLPDELKNEICRYREKSDLLEIEIKNKAIKMLSQEQQDEIKLVNDISSEELKNSVCKKYNIDNSKIPWDKMNGFTTFIADEFINNGGDKSLVYFTAKDKKSKKEKLVKLSDKKIANSFKPKISKETREILNKITWDEKISSNEYKKLSKRKLEFARRATNYLINQAKKATRLNNVVLVVEDLNSKFFHGSGKREDGWDNFFIPKKENRWFIQALHKSLTDVSIHRGINVIEVRPERTSITCPKCGCCDKENRKGEDFKCIKCDSVYHADLEVATFNIEKVAITGESMPKPDCERLGGEESIG

SEQ ID NO: 181 - protein, 661 aa, synthetic construct
  REGION 1..661: note = Description of Artificial Sequence: Synthetic polypeptide
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 182 - protein, 531 aa, synthetic construct
  REGION 1..531: note = Description of Artificial Sequence: Synthetic polypeptide
LEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGIITFSYKEGVVPVFSQKIVSRFKSRDTLEKLTSQGPVALLSVDLGQNEPVAARVCSLKNINDKIALDNSCRIPFLDDYKKQIKDYRDSLDELEIKIRLEAINSLDVNQQVEIRDLDVFSADRAKASTVDMFDIDPNLISWDSMSDARFSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKSFSELSSNRGLCVIEVNPAWTSATCPDCGFCSKENRDGINFTCRKCGVSYHADIDVATLNIARVAVLGKPMSGPADRERLGGTKKPRVARSRKDMKRKDISNGTVEVMVTA

SEQ ID NO: 183 - protein, 479 aa, synthetic construct
  REGION 1..479: note = Description of Artificial Sequence: Synthetic polypeptide
IPSFGYLDRLKIAKGQPGYIPEWQRETINPSKKVRRYWATNHEKIRNAIPLVVFIGDDWVIIDGRGLLRDARRRKLADKNTTIEQLLEMVSNDPVIDSTRGIATLSYVEGVVPVRSFIPIGEKKGREYLEKSTQKESVTLLSVDIGQINPVSCGVYKVSNGCSKIDFLDKFFLDKKHLDAIQKYRTLQDSLEASIVNEALDEIDPSFKKEYQNINSQTSNDVKKSLCTEYNIDPEAISWQDITAHSTLISDYLIDNNITNDVYRTVNKAKYKTNDFGWYKKFSAKLSKEAREALNEKIWELKIASSKYKKLSVRKKEIARTIANDCVKRAETYGDNVVVAMESLTKNNKVMSGRGKRDPGWHNLGQAKVENRWFIQAISSAFEDKATHHGTPVLKVNPAYTSQTCPSCGHCSKDNRSSKDRTIFVCKSCGEKFNADLDVATYNIAHVAFSGKKLSPPSEKSSATKKPRSARKSKKSRKS

SEQ ID NO: 184 - protein, 452 aa, synthetic construct
  REGION 1..452: note = Description of Artificial Sequence: Synthetic polypeptide
SPIEKLLNGLLVKITFGNDWIICDARGLLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 185 - protein, 425 aa, synthetic construct
  REGION 1..425: note = Description of Artificial Sequence: Synthetic polypeptide
LLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 186 - protein, 735 aa, synthetic construct
  REGION 1..735: note = Description of Artificial Sequence: Synthetic polypeptide
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 187 - protein, 18 aa, unidentified
  REGION 1..18: note = Description of Unknown: Nucleoplasmin bipartite NLS sequence
KRPAATKKAGQAKKKKEF

SEQ ID NO: 188 - DNA, 15 bp, synthetic construct
  misc_feature 1..15: note = Description of Artificial Sequence: Synthetic oligonucleotide
  source 1..15: mol_type = other DNA
tcgttgtagctagcc

SEQ ID NO: 189 - DNA, 34 bp, synthetic construct
  misc_feature 1..34: note = Description of Artificial Sequence: Synthetic oligonucleotide
  source 1..34: mol_type = other DNA
aatacaggtaaggctagctacaacgactagcaga

SEQ ID NO: 190 - DNA, 34 bp, synthetic construct
  misc_feature 1..34: note = Description of Artificial Sequence: Synthetic oligonucleotide
  source 1..34: mol_type = other DNA
gcccagggaggctagctacaacgagaggaaacct

SEQ ID NO: 191 - DNA, 34 bp, synthetic construct
  misc_feature 1..34: note = Description of Artificial Sequence: Synthetic oligonucleotide
  source 1..34: mol_type = other DNA
ccaaaggagaggctagctacaacgagggacccgt

SEQ ID NO: 192 - DNA, 32 bp, synthetic construct
  misc_feature 1..32: note = Description of Artificial Sequence: Synthetic oligonucleotide
  source 1..32: mol_type = other DNA
agctggggaggctagctacaacgagagggagg

SEQ ID NO: 193 - DNA, 69 bp, synthetic construct
  misc_feature 1..69: note = Description of Artificial Sequence: Synthetic oligonucleotide
  source 1..69: mol_type = other DNA
gcccagggaggctagctacaacgagaggaaaccttttttaggtttcctctcgttgtagcctccctgggc

SEQ ID NO: 194 - DNA, 71 bp, synthetic construct
  misc_feature 1..71: note = Description of Artificial Sequence: Synthetic oligonucleotide
  misc_feature 1..71: note = Description of Combined DNA/RNA Molecule: Synthetic oligonucleotide
  source 1..71: mol_type = other DNA
  misc_feature 56..57: note = RNA
  misc_feature 1..55: note = DNA
  misc_feature 58..71: note = DNA
gcccagggaggctagctacaacgagaggaaaccttttttaggtttcctctcgttgtttagcctccctgggc

SEQ ID NO: 195 - DNA, 74 bp, synthetic construct
  misc_feature 1..74: note = Description of Artificial Sequence: Synthetic oligonucleotide
  misc_feature 1..74: note = Description of Combined DNA/RNA Molecule: Synthetic oligonucleotide
  source 1..74: mol_type = other DNA
  misc_feature 56..60: note = RNA
  misc_feature 1..55: note = DNA
  misc_feature 61..74: note = DNA
gcccagggaggctagctacaacgagaggaaaccttttttaggtttcctctcgttgttttttagcctccctgggc

SEQ ID NO: 196 - DNA, 51 bp, synthetic construct
  misc_feature 1..51: note = Description of Artificial Sequence: Synthetic oligonucleotide
  misc_feature 1..51: note = Description of Combined DNA/RNA Molecule: Synthetic oligonucleotide
  source 1..51: mol_type = other DNA
  misc_feature 24..28: note = RNA
  misc_feature 1..23: note = DNA
  misc_feature 29..51: note = DNA
agcctccctgggcatcgggtccctttttctcctttgtaaggtttcctctcg

SEQ ID NO: 197 - DNA, 48 bp, synthetic construct
  misc_feature 1..48: note = Description of Artificial Sequence: Synthetic oligonucleotide
  misc_feature 1..48: note = Description of Combined DNA/RNA Molecule: Synthetic oligonucleotide
  source 1..48: mol_type = other DNA
  misc_feature 24..25: note = RNA
  misc_feature 1..23: note = DNA
  misc_feature 26..48: note = DNA
agcctccctgggcatcgggtcccgtctcctttgtaaggtttcctctcg

SEQ ID NO: 198 - DNA, 60 bp, synthetic construct
  misc_feature 1..60: note = Description of Artificial Sequence: Synthetic oligonucleotide
  misc_feature 1..60: note = Description of Combined DNA/RNA Molecule: Synthetic oligonucleotide
  source 1..60: mol_type = other DNA
  misc_feature 19..23: note = RNA
  misc_feature 36..37: note = RNA
  misc_feature 1..18: note = DNA
  misc_feature 24..35: note = DNA
  misc_feature 38..60: note = DNA
agcctccccagctatcactttttagctcctccctcgtccccagcttcctccctctcgttg

SEQ ID NO: 199 - DNA, 60 bp, synthetic construct
  misc_feature 1..60: note = Description of Artificial Sequence: Synthetic oligonucleotide
  misc_feature 1..60: note = Description of Combined DNA/RNA Molecule: Synthetic oligonucleotide
  source 1..60: mol_type = other DNA
  misc_feature 19..23: note = RNA
  misc_feature 35..36: note = RNA
  misc_feature 1..18: note = DNA
  misc_feature 24..34: note = DNA
  misc_RNA 37..60: note = DNA
agcctccccagctatcactttttagctcgaccccgtctccacgcctcctccctctcgttg

SEQ ID NO: 200 - DNA, 41 bp, synthetic construct
  misc_feature 1..41: note = Description of Artificial Sequence: Synthetic oligonucleotide
  misc_feature 1..41: note = Description of Combined DNA/RNA Molecule: Synthetic oligonucleotide
  source 1..41: mol_type = other DNA
  misc_feature 19..23: note = RNA
  misc_feature 1..18: note = DNA
  misc_feature 24..41: note = DNA
agcctccccagctatcactttttagctcctccctctcgttg

SEQ ID NO: 201 - DNA, 57 bp, synthetic construct
  misc_feature 1..57: note = Description of Artificial Sequence: Synthetic oligonucleotide
  misc_feature 1..57: note = Description of Combined DNA/RNA Molecule: Synthetic oligonucleotide
  source 1..57: mol_type = other DNA
  misc_feature 19..23: note = RNA
  misc_feature 37..38: note = RNA
  misc_feature 1..18: note = DNA
  misc_feature 24..36: note = DNA
  misc_feature 39..57: note = DNA
tagccttcctcccatcactttttagctggtttcctcgtccctgggcacgcctctcgt

SEQ ID NO: 202 - DNA, 67 bp, synthetic construct
  misc_feature 1..67: note = Description of Artificial Sequence: Synthetic oligonucleotide
  misc_feature 1..67: note = Description of Combined DNA/RNA Molecule: Synthetic oligonucleotide
  source 1..67: mol_type = other DNA
  misc_feature 23..27: note = RNA
  misc_feature 45..46: note = RNA
  misc_feature 1..22: note = DNA
  misc_feature 28..44: note = DNA
  misc_feature 47..67: note = DNA
ctagcctccctggtcgggtccctttttctcctttgtcacgcctcgttcctcccagtttcctctcgtt

SEQ ID NO: 203 - DNA, 38 bp, synthetic construct
  misc_feature 1..38: note = Description of Artificial Sequence: Synthetic oligonucleotide
  misc_feature 1..38: note = Description of Combined DNA/RNA Molecule: Synthetic oligonucleotide
  source 1..38: mol_type = other DNA
  misc_feature 19..20: note = RNA
  misc_feature 1..18: note = DNA
  misc_feature 21..38: note = DNA
ccagcctttttctgctagattacctgtattttggctgg

SEQ ID NO: 204 - DNA, 22 bp, synthetic construct
  misc_feature 1..22: note = Description of Artificial Sequence: Synthetic oligonucleotide
  misc_feature 1..22: note = Description of Combined DNA/RNA Molecule: Synthetic oligonucleotide
  source 1..22: mol_type = other DNA
  misc_feature 12..13: note = RNA
  misc_feature 1..11: note = DNA
  misc_feature 14..22: note = DNA
aaggtttcctcgtccctgggca

SEQ ID NO: 205 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 11..12: note = RNA
  misc_feature 1..10: note = DNA
  misc_feature 13..21: note = DNA
ctcctccctcgtccccagctc

SEQ ID NO: 206 - RNA, 50 nt, synthetic construct
  misc_feature 1..50: note = Description of Unknown: Target sequence
  source 1..50: mol_type = other RNA
cgctgatggtactatatacaagagtatgggagagtaggtcgtcgccaagc

SEQ ID NO: 207 - RNA, 51 nt, synthetic construct
  misc_feature 1..51: note = Description of Artificial Sequence: Synthetic oligonucleotide
  source 1..51: mol_type = other RNA
ggccaccccaaaaatgaaggggactaaaacacgacctactctcccatactc

SEQ ID NO: 208 - protein, 1307 aa, synthetic construct
  REGION 1..1307: note = Description of Artificial Sequence: Synthetic polypeptide
MADLSQFTHKYQVPKTLRFELIPQGKTLENLSAYGMVADDKQRSENYKKLKPVIDRIYKYFIEESLKNTNLDWNPLYEAIREYRKEKTTATITNLKEQQDICRRAIASRFEGKVPDKGDKSVKDFNKKQSKLFKELFGKELFTDSVLEQLPGVSLSDEDKALLKSFDKFTTYFVGFYDNRKNVFSSDDISTGIPHRLVQENFPKFIDNCDDYKRLVLVAPELKEKLEKAAEATKIFEDVSLDEIFSIKFYNRLLQQNQIDQFNQLLGGIAGAPGTPKIQGLNETLNLSMQQDKTLEQKLKSVPHRFSPLYKQILSDRSSLSFIPESFSCDAEVLLAVQEYLDNLKTEHVIEDLKEVFNRLTTLDLKHIYVNSTKVTAFSQALFGDWNLCREQLRVYKMSNGNEKITKKALGELESWLKNSDIAFTELQEALADEALPAKVNLKVQEAISGLNEQMAKSLPKELKIPEEKEELKALLDAIQEVYHTLEWFIVSDDVETDTDFYVPLKETLQIIQPIIPLYNKVRNFATQKPYSVEKFKLNFANPTLADGWDENKEQQNCAVLFQKGNNYYLGILNPKNKPDFDNVDTEKQGNCYQKMVYKQFPDFSKMMPKCTTQLKEVKQHFEGKDSDYILNNKNFIKPLTITREVYDLNNVLYDGKKKFQIDYLRKTKDEDGYYTALHTWIDFAKKFVASYKSTSIYDTSTILPPEKYEKLNEFYGALDNLFYQIKFENIPEEIIDTYVEDGKLFLFQIYNKDFAAGATGAPNLHTIYWKAVFDPENVKDVVVKLNGQAELFYRPKSNMDVIRHKVGEKLVNRTLKDGSILTDELHKELYLYANGSLKKGLSEDAKIILDKNLAVIYDVHHEIVKDRRFTTDKFFFHVPLTLNYKCDKNPVKFNAEVQEYLKENPDTYVIGIDRGERNLIYAVVIDPKGRIVEQKSFNVINGFDYHGKLDQREKERVKARQAWTAVGKIKELKQGYLSLVVHEISKMMVRYQAVVVLENLNVGFKRVRSGIAEKAVYQQFEKMLINKLNYLMFKDAGGTEPGSVLNAYQLTDRFESFAKMGLQTGFLFYIPAAFTSKIDPATGFVDPFRWGAIKTLADKREFLSGFESLKFDSTTGNFILHFDVSKNKNFQKKLEGFVPDWDIIIEANKMKTGKGATYIAGKRIEFVRDNNSQGHYEDYLPCNALAETLRQCDIPYEEGKDILPLILEKNDSKLLHSVFKVVRLTLQMRNSNAETGEDYISSPVEDVSGSCFDSRMENEKLPKDADANGAYHIALKGMLALERLRKDEKMAISNNDWLNYIQEKRA

SEQ ID NO: 209 - protein, 492 aa, synthetic construct
  REGION 1..492: note = Description of Artificial Sequence: Synthetic polypeptide
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 210 - protein, 529 aa, synthetic construct; identical to SEQ ID NO: 49
  REGION 1..529: note = Description of Artificial Sequence: Synthetic polypeptide

SEQ ID NO: 211 - protein, 1297 aa, synthetic construct
  REGION 1..1297: note = Description of Artificial Sequence: Synthetic polypeptide
MATLVSFTKQYQVQKTLRFELIPQGKTQANIDAKGFINDDLKRDENYMKVKGVIDELHKNFIEQTLVNVDYDWRSLATAIKNYRKDRSDTNKKNLEKTQEAARKEIIAWFEGKRGNSAFKNNQKSFYGKLFKKELFSEILRSDDLEYDEETQDAIACFDKFTTYFVGFHENRKNMYSTEAKSTSVAYRVVNENFSKFLSNCEAFSVLEAVCPNVLVEAEQELHLHKAFSDLKLSDVFKVEAYNKYLSQTGIDYYNQIIGGISSAEGVRKIRGVNEVVNNAIQQNDELKVALRNKQFTMVQLFKQILSDRSTLSFVSEQFTSDQEVITVVKQFNDDIVNNKVLAVVKTLFENFNSYDLEKIYINSKELASVSNALLKDWSKIRNAVLENKIIELGANPPKTKISAVEKEVKNKDFSIAELASYNDKYLDKEGNDKEICSIANVVLEAVGALEIMLAESLPADLKTLENKNKVKGILDAYENLLHLLNYFKVSAVNDVDLAFYGAFEKVYVDISGVMPLYNKVRNYATKKPYSVEKFKLNFAMPTLADGWDKNKERDNGSIILLKDGQYYLGVMNPQNKPVIDNAVCNDAKGYQKMVYKMFPEISKMVTKCSTQLNAVKAHFEDNTNDFVLDDTDKFISDLTITKEIYDLNNVLYDGKKKFQIDYLRNTGDFAGYHKALETWIDFVKEFLSKYRSTAIYDLTTLLPTNYYEKLDVFYSDVNNLCYKIDYENISVEQVNEWVEEGNLYLFKIYNKDFATGSTGKPNLHTMYWNAVFAEENLHDVVVKLNGGAELFYRPKSNMPKVEHRVGEKLVNRKNVNGEPIADSVHKEIYAYANGKISKSELSENAQEELPLAIIKDVKHNITKDKRYLSDKYFFHVPITLNYKANGNPSAFNTKVQAFLKNNPDVNIIGIDRGERNLLYVVVIDQQGNIIDKKQVSYNKVNGYDYYEKLNQREKERIEARQSWGAVGKIKELKEGYLSLVVREIADMMVKYNAIVVMENLNAGFKRVRGGIAEKAVYQKFEKMLIDKLNYLVFKDVEAKEAGGVLNAYQLTDKFDSFEKMGNQSGFLFYVPAAYTSKIDPVTGFANVFSTKHITNTEAKKEFICSFNSLRYDEAKDKFVLECDLNKFKIVANSHIKNWKFIIGGKRIVYNSKNKTYMEKYPCEDLKATLNASGIDFSSSEIINLLKNVPANREYGKLFDETYWAIMNTLQMRNSNALTGEDYIISAVADDNEKVFDSRTCGAELPKDADANGAYHIALKGLYLLQRIDISEEGEKVDLSIKNEEWFKFVQQKEYAR

SEQ ID NO: 212 - protein, 1145 aa, synthetic construct
  REGION 1..1145: note = Description of Artificial Sequence: Synthetic polypeptide
MCMKITKIDGISHKKYKEKGKLIKNNDTAKDIIEERFNDIEKKTKELFQKTLDFYVKNYEKCKEQNKERREKAKNYFSKVKILVDNKKITICNENTEKMEIEDFNEYDVRSGKYFNVLNKILNGENYTEEDLEVFENDLQKRTGRIKSIKNSLEENKAHFKKESINNNIIYDRVKGNNKKSLFYEYYRISSKHQEYVNNIFEAFDKLYSNSHEAMNNLFSEITKDSKDRNIRKIREAYHEILNKNKTEFGEELYKKIQDNRNNFDKLLEIEPEIKELTKSQIFYKYYIDKVNLDETSIKHCFCHLVEIEVNQLLKNYVYSKRNINKEKLENIFEYCKLKNLIKNKLVNKLNNYIRNCGKYNAYISNNDVVVNSEKISEIRTKEAFLRSIIGVSSSAYFSLRNILNTDNTQDITNKVDKEVDKLYQENKKIELEERLKLFFGNYFDINNQQEIKVFLMNIDKIISSIRHEIIHFKMETNAQNIFDFNNVNLGNTAKNIFSNEINEEKIKFKIFKQLNSANVFDYLSNKDITEYMDKVVFSFTNRNVSFVPSFTKIYNRVQDLANSLEIKKWKIPDKSEGKDAQIYLLKNIYYGKFLDEFLNEENGIFISIKDKIIELNRNQNKRTGFYKLEKFEKIEETNPKKYLEIIQSLYMINIEEIDSEGKNIFLDFIQKIFLKGFFEFIKNNYNYLLELKKIQDKKNIFDSEMSEYIAGEKTLEDIGEINEIIQDIKITEIDKILNQTDKINCFYLLLKLLNYKEITELKGNLEKYQILSKTNVYEKELMLLNIVNLDNNKVKIENFKILAEEIGKFIEKINIEEINKNKKIKTFEELRNFEKGENTGEYYNIYSDDKNIKNIRNLYNIKKYGMLDLLEKISEKTNYCIKKKDLEEYSELRKQLEDEKTNFYKIQEYLHSKYQQKPKKILLKNNKNDYEKYKKSIENIEKYVHLKNKIEFNELNLLQSLLLKILHRLVGFTSIWERDLRFRLIGEFPDELDVEDIFDHRKRYKGTGKGICKKYDRFINTHTEYKNNNKMENVKFADNNPVRNYIAHFNYLPNPKYSILKMMEKLRKLLDYDRKLKNAVMKSIKDILEEYGFKAEFIINSDKEIILNLVKSVEIIHLGKEDLKSRRNSEDLCKLVKAMLEYSK

SEQ ID NO: 213 - protein, 1271 aa, synthetic construct
  REGION 1..1271: note = Description of Artificial Sequence: Synthetic polypeptide
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKRLEKIQEKKRKEIYQYLSDDAVFSEMFKEKMISGILPDFIRCNEGYSEEEKEEKLKTVALFHRFTSSFNDFFLNRKNVFTKEAIVTAIGYRVVHENAEIFLENMVAFQNIQKSAESQISIIERKNEHYFMEWKLSHIFTADYYMMLMTQKAIEHYNEMCGVVNQQMREYCQKEKKNWNLYRMKRLHKQILSNASTSFKIPEKYENDAEVYESVNSFLQNVMEKTVMERIAVLKNSTDNFDLSKIYITAPYYEKISNYLCGSWNTITDCLTHYYEQQIAGKGARKDQKVKAAVKADKWKSLSEIEQLLKEYARAEEVKRKPEEYIAEIENIVSLKEAHLLEYHPEVNLIENEKYATEIKDVLDNYMELFHWMKWFYIEEAVEKEVNFYGELDDLYEEIKDIVPLYNKVRNYVTQKPYSDTKIKLNFGTPTLANGWSKSKEYDYNAILLQKDGKYYMGIFNPIQKPEKEIIEGHSQPLEGNEYKKMVYYYLPSANKMLPKVLLSKKGMEIYQPSEYIINGYKERRHIKSEEKFDLQFCHDLIDYFKSGIERNSDWKVFGFDFSDTDTYQDISGFYREVEDQGYKIDWTYIKEADIDRLNEEGKLYLFQIYNKDFSEKSTGRENLHTMYLKNLFSEENVREQVLKLNGEAEIFFRKSSVKKPIIHKKGTMLVNRTYMEEVNGNSVRRNIPEKEYQEIYNYKNHRLKGELSTEAKKYLEKAVCHETKKDIVKDYRYSVDKFFIHLPITINYRASGKETLNSVAQRYIAHQNDMHVIGIDRGERNLIYVSVINMQGEIKEQKSFNIINEFNYKEKLKEREQSRGAARRNWKEIGQIKDLKEGYLSGVIHEIAKMMIKYHAIIAMEDLNYGFKRGRFKVERQVYQKFENMLIQKLNYLVFKDRPADEDGGVLRGYQLAYIPDSVKKMGRQCGMIFYVPAAFTSKIDPTTGFVDIFKHKVYTTEQAKREFILSFDEICYDVERQLFRFTFDYANFVTQNVTLARNNWTIYTNGTRAQKEFGNGRMRDKEDYNPKDKMVELLESEGIEFKSGKNLLPALKKVSNAKVFEELQKIVRFTVQLRNSKSEENDVDYDHVISPVLNEEGNFFDSSKYKNKEEKKESLLPVDADANGAYCIALKGLYIMQAIQKNWSEEKALSPDVLRLNNNDWFDYIQNKRYR

SEQ ID NO: 214 - protein, 1229 aa, synthetic construct
  REGION 1..1229: note = Description of Artificial Sequence: Synthetic polypeptide
MEEKKMSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 215 - protein, 835 aa, synthetic construct
  REGION 1..835: note = Description of Artificial Sequence: Synthetic polypeptide
MEKIKKPSNRNSIPSIIISDYDANKIKEIKVKYLKLARLDKITIQDMEIVDNIVEFKKILLNGVEHTIIDNQKIEFDNYEITGCIKPSNKRRDGRISQAKYVVTITDKYLRENEKEKRFKSTERELPNNTLLSRYKQISGFDTLTSKDIYKIKRYIDFKNEMLFYFQFIEEFFNPLLPKGKNFYDLNIEQNKDKVAKFIVYRLNDDFKNKSLNSYITDTCMIINDFKKIQKILSDFRHALAHFDFDFIQKFFDDQLDKNKFDINTISLIETLLDQKEEKNYQEKNNYIDDNDILTIFDEKGSKFSKLHNFYTKISQKKPAFNKLINSFLSQDGVPNEEFKSYLVTKKLDFFEDIHSNKEYKKIYIQHKNLVIKKQKEESQEKPDGQKLKNYNDELQKLKDEMNTITKQNSLNRLEVKLRLAFGFIANEYNYNFKNFNDEFTNDVKNEQKIKAFKNSSNEKLKEYFESTFIEKRFFHFSVNFFNKKTKKEETKQKNIFNSIENETLEELVKESPLLQIITLLYLFIPRELQGEFVGFILKIYHHTKNITSDTKEDEISIEDAQNSFSLKFKILAKNLRGLQLFHYSLSHNTLYNNKQCFFYEKGNRWQSVYKSFQISHNQDEFDIHLVIPVIKYYINLNKLMGDFEIYALLKYADKNSITVKLSDITSRDDLKYNGHYNFATLLFKTFGIDTNYKQNKVSIQNIKKTRNNLAHQNIENMLKAFENSEIFAQREEIVNYLQTEHRMQEVLHYNPINDFTMKTVQYLKSLSVHSQKEGKIADIHKKESLVPNDYYLIYKLKAIELLKQKVIEVIGESEDEKKIKNAIAKEEQIKKGNN

SEQ ID NO: 216 - protein, 1367 aa, synthetic construct
  REGION 1..1367: note = Description of Artificial Sequence: Synthetic polypeptide
MEKSLNDFIGLYSVSKTLRFELKPVSETLENIKKFHFLEEDKKKANDYKDVKKIIDNYHKYFIDDVLKNASFNWKKLEEAIREYNKNKSDDSALVAEQKKLGDAILKLFTSDKRYKALTAATPKELFESILPDWFGEQCNQDLNKAALKTFQKFTSYFTGFQENRKNVYSAEAIPTAVPYRIVNDNFPKFLQNVLIFKTIQEKCPQIIDEVEKELSSYLGKEKLAGIFTLESFNKYLGQGGKENQRGIDFYNQIIGGVVEKEGGINLRGVNQFLNLYWQQHPDFTKEDRRIKMVPLYKQILSDRSSLSFKIESIENDEELKNALLECADKLELKNDEKKSIFEEVCDLFSSVKNLDLSGIYINRKDINSVSRILTGDWSWLQSRMNVYAEEKFTTKAEKARWQKSLDDEGENKSKGFYSLTDLNEVLEYSSENVAETDIRITDYFEHRCRYYVDKETEMFVQGSELVALSLQEMCDDILKKRKAMNTVLENLSSENKLREKTDDVAVIKEYLDAVQELLHRIKPLKVNGVGDSTFYSVYDSIYSALSEVISVYNKTRNYITKKAASPEKYKLNFDNPTLADGWDLNKEQANTSVILRKDGMFYLGIMNPKNKPKFAEKYDCGNESCYEKMIYKQFDATKQIPKCSTQKKEVQKYFLSGATEPYILNDKKSFKSELIITKDIWFMNNHVWDGEKFVPKRDNETRPKKFQIGYFKQTGDFDGYKNALSNWISFCKNFLQSYLSATVYDYNFKNSEEYEGLDEFYNYLNATCYKLNFINIPETEINKMVSEGKLYLFQIYNKDFASGSTGMPNMHTLYWKNLFSDENLKNVCLKLNGEAELFYRPAGIKEPVIHKEGSYLVNRTTEDGESIPEKIYFEIYKNANGKLEKLSDEAQNYISNHEVVIKKAGHEIIKDRHYTEPKFLFHVPLTINFKASGNSYSINENVRKFLKNNPDVNIIGLDRGERHLIYLSLINQKGEIIKQFTFNEVERNKNGRTIKVNYHEKLDQREKERDAARKSWQAIGKIAELKEGYLSAVIHQLTKLMVEYNAVVVMEDLNFGFKRGRFHVEKQVYQKFEHILIDKSNYLVFKDRGLNEPGGVLNGYQIAGQFESFQKLGKQSGMLFYVPAGYTSKIDPKTGFVSMMNFKDLTNVHKKRDFFSKFDNIHYDEANGSFVFTFDYKKFDGKAKEEMKLTKWSVYSRDKRIVYFAKTKSYEDVLPTEKLQKIFESNGIDYKSGNNIQDSVMAIGADLKEGAKPSKEISDFWDGLLSNFKLILQMRNSNARTGEDYIISPVMADDGTFFDSREEFKKGEDAKLPLDADANGAYHIALKGLSLINKINLSKDEELKKFDMKISNADWFKFAQEKNYAK

SEQ ID NO: 217 - protein, 1264 aa, synthetic construct
  REGION 1..1264: note = Description of Artificial Sequence: Synthetic polypeptide
MENYGGFTGLYPLQKTLKFELRPQGRTMEHLVSSNFFEEDRDRAEKYKIVKKVIDNYHKDFINECLSKRSFDWTPLMKTSEKYYASKEKNGKKKQDLDQKIIPTIENLSEKDRKELELEQKRMRKEIVSVFKEDKRFKYLFSEKLFSILLKDEDYSKEKLTEKEILALKSFNKFSGYFIGLHKNRANFYSEGDESTAIAYRIVNENFPKFLSNLKKYREVCEKYPEIIQDAEQSLAGLNIKMDDIFPMENFNKVMTQDGIDLYNLAIGGKAQALGEKQKGLNEFLNEVNQSYKKGNDRIRMTPLFKQILSERTSYSYILDAFDDNSQLITSINGFFTEVEKDKEGNTFDRAVGLIASYMKYDLSRVYIRKADLNKVSMEIFGSWERLGGLLRIFKSELYGDVNAEKTSKKVDKWLNSGEFSLSDVINAIAGSKSAETFDEYILKMRVARGEIDNALEKIKCINGNFSEDENSKMIIKAILDSVQRLFHLFSSFQVRADFSQDGDFYAEYNEIYEKLFAIVPLYNRVRNYLTKNNLSMKKIKLNFKNPALANGWDLNKEYDNTAVIFLREGKYYLGIMNPSKKKNIKFEEGSGTGPFYKKMAYKLLPDPNKMLPKVFFAKKNINYYNPSDEIVKGYKAGKYKKGENFDIDFCHKLIDFFKESIQKNEDWRAFNYLFSATESYKDISDFYSEVEDQGYRMYFLNVPVANIDEYVEKGDLFLFQIYNKDFASGAKGNKDMHTIYWNAAFSDENLRNVVVKLNGEAELFYRDKSIIEPICHKKGEMLVNRTCFDKTPVPDKIHKELFDYHNGRAKTLSIEAKGYLDRVGVFQASYEIIKDRRYSENKMYFHVPLKLNFKADGKKNLNKMVIEKFLSDKDVHIIGIDRGERNLLYYSVIDRRGNIIDQDSLNIIDGFDYQKKLGQREIERREARQSWNSIGKIKDLKEGYLSKAVHKVSKMVLEYNAIVVLEDLNFGFKRGRFKVEKQVYQKFEKMLIDKLNYLVFKEVLDSRDAGGVLNAYQLTTQLESFNKLGKQSGILFYVPAAYTSKIDPTTGFVSLFNTSRIESDSEKKDFLSGFDSIVYSAKDGGIFAFKFDYRNRNFQREKTDHKNIWTVYTNGDRIKYKGRMKGYEITSPTKRIKDVLSSSGIRYDDGQELRDSIIQSGNKVLINEVYNSFIDTLQMRNSDGEQDYIISPVKNRNGEFFRTDPDRRELPVDADANGAYHIALRGELLMQKIAEDFDPKSDKFTMPKMEHKDWFEFMQTRGD

SEQ ID NO: 218 - protein, 500 aa, synthetic construct; identical to SEQ ID NO: 47
  REGION 1..500: note = Description of Artificial Sequence: Synthetic polypeptide

SEQ ID NO: 219 - protein, 1261 aa, synthetic construct
  REGION 1..1261: note = Description of Artificial Sequence: Synthetic polypeptide
MIIHNCYIGGSFMKKIDSFTNCYSLSKTLRFKLIPIGATQSNFDLNKMLDEDKKRAENYSKAKSIIDKYHRFFIDKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQPTAISYRCINDNLPKFLDNVRTFKNSDVASILNDNLKILNEDFDGIYGTSAEDVFNVDYFPFVLSQKGIEAYNSILGGYTNSDGSKIKGLNEYINLYNQKNENIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSIESFSLADIQKYADVSESSETNAEVTEWLRNEIKEKCNLAVQGYESSKDLISKPYTESKKLFNNDNAVELIKNALDSVKELENVLRLLLGTGKEESKDENFYGEFLPCYERICEVDSLYDKVRNYMTQKLYKTDKIKLNFQNPQFLGGWDRNKEADYSAVLLRRNSLYYIAIMPSGYKRVFEKIPAPKADETVYEKVIYKLLPGPNKMLPKVFFSKKGIETFNPPKEILEKYELGTHKTGDGFNLDDCHALIDYFKSALDVHSDWSNFGFRFSDTSTYKNIADFYNEVKNQGYKITFCDVPQSYINELVDEGKLYLFQLYNKDFSEHSKGTPNLHTLYFKMLFDERNLENVVFKLNGEAEMFYREASISKDDMIVHPKNQPIKNKNEQNSRKQSTFEYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNLGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 220 - protein, 478 aa, synthetic construct
  REGION 1..478: note = Description of Artificial Sequence: Synthetic polypeptide
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 221 - protein, 1229 aa, synthetic construct
  REGION 1..1229: note = Description of Artificial Sequence: Synthetic polypeptide
MKEQFINRYPLSKTLRFSLIPVGETENNFNKNLLLKKDKQRAENYEKVKCYIDRFHKEYIESVLSKARIEKVNEYANLYWKSNKDDSDIKAMESLENDMRKQISKQLTSTEIYKKRLFGKELICEDLPSFLTDKDERETVECFRSFTTYFKGFNTNRENMYSSDGKSTAIAYRCINDNLPRFLDNVKSFQKVFDNLSDETITKLNTDLYNIFGRNIEDIFSVDYFEFVLTQSGIEIYNSMIGGYTCSDKTKIQGLNECINLYNQQVAKNEKSKKLPLMKPLYKQILSEKDSVSFIPEKFNSDNEVLHAIDDYYTGHIGDFDLLTELLQSLNTYNANGIFVKSGVAITDISNGAFNSWNVLRSAWNEKYEALHPVTSKTKIDKYIEKQDKIYKAIKSFSLFELQSLGNENGNEITDWYISSINESNSKIKEAYLQAQKLLNSDYEKSYNKRLYKNEKATELVKNLLDAIKEFQKLIKPLNGTGKEENKDELFYGKFTSYYDSIADIDRLYDKVRNYITQKPYSKDKIKLNFDNPQLLGGWDKNKESDYRTVLLHKDGLYYLAVMDKSHSKAFVDAPEITSDDKDYYEKMEYKLLPGPNKMLPKVFFASKNIDTFQPSDRILDIRKRESFKKGATFNKAECHEFIDYFKDSIKKHDDWSQFGFKFSPTESYNDISEFYREISDQGYSVRFNKISKNYIDGLVNNGYIYLFQIYNKDFSKYSKGTPNLHTLYFKMLFDERNLSNVVYKLNGEAEMFYREASIGDKEKITHYANQPIKNKNPDNEKKESVFEYDIVKDKRFTKRQFSLHLPITINFKAHGQEFLNYDVRKAVKYKDDNYVIGIDRGERNLIYISVINSNGEIVEQMSLNEIISDNGHKVDYQKLLDTKEKERDKARKNWTSVENIKELKEGYISQVVHKICELVIKYDAVIAMEDLNFGFKRGRFPVEKQVYQKFENMLISKLNLLIDKKAEPTEDGGLLRAYQLTNKFDGVNKAKQNGIIFYVPAWDTSKIDPATGFVNLLKPKCNTSVPEAKKLFETIDDIKYNANTDMFEFYIDYSKFPRCNSDFKKSWTVCTNSSRILTFRNKEKNNKWDNKQIVLTDEFKSLFNEFGIDYKGNLKDSILSISNADFYRRLIKLLSLTLQMRNSITGSTLPEDDYLISPVANKSGEFYDSRNYKGTNAALPCDADANGAYNIARKALWAINVLKDTPDDMLNKAKLSITNAEWLEYTQK

SEQ ID NO: 222 - protein, 1242 aa, synthetic construct
  REGION 1..1242: note = Description of Artificial Sequence: Synthetic polypeptide
MKEQFVNQYPISKTLRFSLIPIGKTEENFNKNLLLKEDEKKAEEYQKVKGYIDRYHKFFIETALCNINFEGFEEYSLLYYKCSKDDNDLKTMEDIEIKLRKQISKTMTSHKLYKDLFGENMIKTILPNFLDSDEEKNSLEMFRGFYTYFSGFNTNRKNMYTEEAKSTSIAYRCINDNLPKFLDNSKSFEKIKCALNKEELKAKNEEFYEIFQIYATDIFNIDFFNFVLTQPGIDKYNGIIGGYTCSDGTKVQGLNEIINLYNQQIAKDDKSKRLPLLKMLYKQILSDRETVSFIPEKFSSDNEVLESINNYFSKNVSNAIKSLKELFQGFEAYNMNGIFISSGVAITDLSNAVFGDWNAISTAWEKAYFETNPPKKNKSQEKYEEELKANYKKIKSFSLDEIQRLGSIAKSPDSIGSVAEYYKITVTEKIDNITELYDGSKELLNCNYSESYDKKLIKNDTVIEKVKTLLDAVKSLEKLIKPLVGTGKEDKDELFYGTFLPLYTSLSAVDRLYDKVRNYATQKPYSKDKIKLNFNCSSFLSGWATDYSSNGGLIFEKDGLYYLGIVNKKFTTEEIDYLQQNADENPAQRIVYDFQKPDNKNTPRLFIRSKGTNYSPSVKEYNLPVEEIVELYDKRYFTTEYRNKNPELYKASLVKLIDYFKLGFTRHESYRHYDFKWKKSEEYNDISEFYKDVEISCYSLKQEKINYNTLLNFVAENRIYLFQIYNKDFSKYSKGTPNLHTRYFKALFDENNLSDVVFKLNGGSEMFFRKASIKDNEKVVHPANQPIDNKNPDNSKKQSTFDYELIKDKRFTKHQFSIHIPITMNFKARGRDFINNDIRKAIKSEYKPYVIGIDRGERNLIYISVINNNGEIVEQMSLNDIISDNGYKVDYQRLLDRKEKERDNARKSWGTIENIKELKEGYISQVIHKICELVIKYDAVIAMEDLNFGFKRGRFNVEKQVYQKFENMLISKLNYLCDKKSEANSEGGLLKAYQLTNKFDGVNKGKQNGIIFYVPAWLTSKIDPVTGFVDLLHPKYISVEETHSLFEKLDDIRYNFEKDMFEFDIDYSKLPKCNADFKQKWTVCTNADRIMTFRNSEKNNEWDNKRILLSDEFKRLFEEFGIDYCHNLKNKILSISNKDFCYRFIKLFALTMQMRNSITGSTNPEDDYLISPVRDENGVFYDSRNFIGSKAGLPIDADANGAYNIARKGLWAINAIKSTADDMLDKVDLSISNAKWLEYVQK

SEQ ID NO: 223 - protein, 1168 aa, synthetic construct
  REGION 1..1168: note = Description of Artificial Sequence: Synthetic polypeptide
MKITKIDGILHKKYIKEGKLVKSTSEENKTDERLSELLTIRLDTYIKNPDNASEEENRIRRETLKEFFSNKVLYLKDSILYLKDRREKNQLQNKNYSEEDISEYDLKNKNSFLVLKKILLNEDINSEELEIFRNDFEKKLDKINSLKYSLEENKANYQKINENNIKKVEGKSKRNIFYNYYKDSAKRNDYINNIQEAFDKLYKKEDIENLFFLIENSKKHEKYKIRECYHKIIGRKNDKENFATIIYEEIQNVNNMKELIEKVPNVSELKKSQVFYKYYLNKEKLNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSTAYFSLRNILETENENDITGRIKGKTVKNKKGEEKYISGEIDKLYDNNKQNEVKKNLKMFYSYDFNMNRKKEIEDFFSNIDEAISSIRHGIVHFNLELEGKDIFTFKNIVPSQISKKMFQNEINEKKLKLKIFRQLNSANVFRYLEKYKILNYLNRTRFEFVNKNIPFVPSFTKLYSRIDDLKNSLCIYWKIPKANDNNKTKEITDAQIYLLKNIYYGEFLNYFMSNNGNFFEIIKEIIELNKNDKRNLKTGFYKLQKFENLQEKTPKEYLANIQSFYMIDAGNKDEEEKDAYIDFIQKIFLKGFMTYLANNGRLSLMYIGNDEQINTSLAGKKQEFDKFLKKYEQNNNIEIPHEINEFVREIKLGKILKYTESLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGILNLLEKISDEAKYKISIEELKNYSNKKIEIEKNHTTQENLHRKYARPRKDEKFNDEDYKKYEKTIRNIQQYTHLKNKVEFNELNLLQSLLLRILHRLVGYTSIWERDLRFRLKGEFPENQYIEEIFNFDNSKNVKYKNGQIVEKYISFYKELYKDDMEKISIYSDKKVKELKKEKKDLYIRNYIAHFNYIPNAEVSLLEVLENLRKLLSYDRKLKNAIMKSIVDILKEYGFVVTFKIEKDKKIRIESLKSEEVVHLKKLKLKDNDKKKEPIKTYRNSKELCKLVKVMFEYKMKEKKSEN

SEQ ID NO: 224 - protein, 1168 aa, synthetic construct
  REGION 1..1168: note = Description of Artificial Sequence: Synthetic polypeptide
MKITKIDGISHKKYIKEGKLVKSTSEENKTDERLSELLTIRLDTYIKNPDNASEEENRIRRENLKEFFSNKVLYLKDGILYLKDRREKNQLQNKNYSEEDISEYDLKNKNSFLVLKKILLNEDINSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIRECYHKIIGRKNDKENFAKIIYEEIQNVNNIKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIAGNRQNEAFLRNIIGVSSVAYFSLRNILETENKDDITGKMRGKTRIDSKTGEEKYIPGEVDQIYYENKQNEVKNKLKMFYGYDFDMDNKKEIEDFFANIDEAISSIRHGIVHFNLDLDGKDIFAFKNIVPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISREIIELNKNDKRNLKTGFYKLQKFEDIQEKTPKKYLANIQSLYMINAGNQDEEEKDTYIDFIQKIFLKGFMTYLANNGRLSLMYIGNDEQINTSLAGKKQEFDKFLKKYEQNNNIEIPHEINEFLREIKLGKILKYTESLNMFYLILKLLNHKELTNLKGSLEKYQSANKEETFSDELELINLLNLDNNRVTEDFELEANEIGKFLDFNGNKIKDRKELKKFDTKKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENQYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSVVDILKEYGFVATFKIGADKKIGIQTLESEKIVHLKNLKKKKLMTDRNSEELCKLVKVMFEYKMEEKNLKTKKCKVI

SEQ ID NO: 225 - protein, 1224 aa, synthetic construct; identical to SEQ ID NO: 46
  REGION 1..1224: note = Description of Artificial Sequence: Synthetic polypeptide

SEQ ID NO: 226 - protein, 1225 aa, synthetic construct
  REGION 1..1225: note = Description of Artificial Sequence: Synthetic polypeptide
MKKIDSFVNYYPLSKTLRFSLIPVGKTEDNFNAKLLLEEDEKRAIEYEKVKRYIDRYHKHFIETVLANFHLDDVNEYAELYYKAGKDDKDLKYMEKLEGKMRKSISAAFTKDKKYKEIFGQEIIKNILPEFLENEDEKESVKMFQGFFTYFTGFNDNRKNMYTHEAQTTAISYRCINENLPKFLDNVQSFAKIKESISSDIMNKLDEVCMDLYGVYAQDMFCTDYFSFVLSQSGIDRYNNIIGGYVDDKGVKIQGINEYINLYNQQVDEKNKRLPLMKKLYKQILIEKESISFIPEKFESDNIVINAISDYYHNNVENLFDDFNKLFNEFSEYDDNGIFVTSGLAVTDISNAVFGSWNIISDSWNEEYKDSHPMKKTTNAEKYYEDMKKEYKKNLSFTIAELQRLGEAGCNDECKGDIKEYYKTTVAEKIENIKNAYEISKDLLASDYEKSNDKKLCKNDSAISLLKNLLDSIKDLEKTIKPLLGTGKEENKDDVFYGKFTNLYEMISEIDRLYDKVRNYVTQKPYSKDKIKLNFENPQHLGGWDKNKERDYRSVLLKKEDKYYLAIMDKSNNKAFIDFPDDGECYEKIEYKLLPGPNKMLPKVFFASSNIEYFAPSKKILEIRSRESFKKGDMFNLKDCHEFIDFFKESIKKHEDWSQFGFEFSPTEKYNDISEFYNEVKIQGYSLKYKNVSKKYIDELIECGQLYLFQIYNKDFSVYAKGNPNLHTMYFKMLFDERNLANVVYQLNGGAEMFYRKASIKDSEKIVHHANQPIKNKNADNVKKESVFEYDIIKDKRFTKRQFSIHIPITLNFKAKGQNFINNDVRMALKKADENYVIGIDRGERNLLYICVINSKGEIVEQKSLNEIIGDNGYRVDYHKLLDKKEAERDEARKSWGTIENIKELKEGYLSQIVHEISKLVIKYDAVIAIEDLNSGFKKGRFKVEKQVYQKFENMLCTKLNYLVDKNADANECGGLLKAYQLTNKEDGANRGRQNGIIFSVPAWLTSKIDPVTGFADLLRPKYKSVSESVEFISKIDNIRYNSKEDYFEFDIDYSKFPNSTASYKKKWTVCTYGERIINVRNKEKNNMWDNKTIVLTDEFKKLFADFGVDVSKNIKESVLAIDSKDFYYRFINLLANTLQLRNSEVGNVDVDYLISPVKGVDGSFYDSRLVKEKTLPENADANGAYNIARKALWAIDVLKQTKDEELKNANLSIKNAEWLEYVQK

SEQ ID NO: 227 - protein, 1165 aa, synthetic construct
  REGION 1..1165: note = Description of Artificial Sequence: Synthetic polypeptide
MKNQNTLPSNPTDILKDKPFWAAFFNLARHNVYLTVNHINKLLDLEKLYNKDKHKEIFEHEDIFNISDDVMNDVNSNGKKRKLDIKKIWANLDTDLTRKYQLRELILKHFPFIQPAIIGAQTKERTTIDKDKRSTSTSNDSLKPTGEGDINDPLSLSNVKSIFFRLLQMLEQLRNYYSHVKHSKSATMPNFDEGLLKSMYNIFIDSVNKVKEDYSSNSVIDPNTSFSHLISKDEQGEIKPCRYSFTSKDGSINASGLLFFVSLFLEKQDSIWMQKKIPGFKKTSENYMKMTNEVFCRNHILLPKMRLETVYDKDWMLLDMLNEVVRCPLSLYKRLAPADQNKFKVPEKSSDNANRQEDDNPFSRILVRHQNRFPYFALRFFDLNEVFTTLRFQINLGCYHFAICKKQIGDKKEVHHLTRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLERLNKQPEQKPNVAAKNTGTLLRNGQIADWLVKDMMRFQPVKRDKEGNPINCSKANSTEYQMLQRAFAFYTTDSYRLPRYFEQLHLINCDNSHLFLSRFEYDKQPNLIAFYAAYLEAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYTYNFNVGNVSKITEANYLSKEKRENLFKSYQNKFKNNIPAEKTKEYREYKNFSSWKKFERELRLIKNQDILTWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDINLEKYPLTKYQVDSELDLYQKYRIDIFKQTLDLEAQLLDKYSDLNTDNFNQMLSGWSEKEGIPRNIKQDVAFLIGVRNGFSHNQYPDSKRIAFSRIKKFNPKTSSLQESEGLNIAKQMYEEAQQVVNKIKNIESFD

SEQ ID NO: 228 - protein, 1194 aa, synthetic construct
  REGION 1..1194: note = Description of Artificial Sequence: Synthetic polypeptide
MKVTKIDGISHKKFEDEGKLVKFTGHFNIKNEMKERLEKLKELKLSNYIKNPENVKNKDKNKEKETKSRRENLKKYFSEIILRKKEEKYLLKKTRKFKNITEEINYDDIKKRENQQKIFDVLKELLEQRINENDKEEILNFDSVKLKEAFEEDFIKKELKIKAIEESLEKNRADYRKDYVELENEKYEDVKGQNKRSLVFEYYKNPENREKFKENIKYAFENLYTEENIKNLYSEIKEIFEKVHLKSKVRYFYQNEIIGESEFSEKDEEGISILYKQIINSVEKKEKFIEFLQKVKIKDLTRSQIFYKYFLENEELNDENIKYVFSYFVEIEVNKLLKENVYKTKKFNEGNKYRVKNIFNYDKLKNLVVYKLENKLNNYVRNCGKYNYHMENGDIATSDINMKNRQTEAFLRSILGVSSFGYFSLRNILGVNDDDFYKIEKDERKNENFILKKAKEDFTSKNIFEKVVDKSFEKKGIYQIKENLKMFYGNSFDKVDKDELKKFFVNMLEAITSVRHRIVHYNINTNSENIFDFSNIEVSKLLKNIFEKEIDTRELKLKIFRQLNSAGVFDYWESWVIKKYLENVKFEFVNKNVPFVPSFKKLYDRIDNLKGWNALKLGNNINIPKRKEAKDSQIYLLKNIYYGEFVEKFVNDNKNFEKIVKEIIEINRGAGTNKKTGFYKLEKFETLKANTPTKYLEKLQSLHKISYDKEKIEEDKDVYVDFVQKIFLKGFVNYLKKLDSLKSLNLLNLRKDETITDKKSVHDEKLKLWENSGSNLSKMPEEIYEYVKKIKISNINYNDRMSIFYLLLKLIDYRELTNLRGNLEKYESMNKNKIYSEELTIINLVNLDNNKVRTNFSLEAEDIGKFLKSSITIKNIAQLNNFSKIFADGENVIKHRSFYNIKKYGILDLLEKIVAKADLKITKEEIKKYENLQNELKRNDFYKIQEQIHRNYNQKPFSIKKIENKKDFEKYKKVIEKIQDYTQLKNKIEFNDLNLLQSLIFRILHRLAGYTSLWERDLQFKLKGEFPEDKYIDEIFNSDGNNNQKYKHGGIADKYANFLIEKKEEKSGEILNKKQRKKKIKEDLEIRNYIAHFNYLPNAEKSILEILEELRELLKHDRKLKNAVMKSIKDIFREYGFIVEFTISHTKNGKKIKVCSVKSEKIKHLKNNELITTRNSEDLCELVKIMLEHKELQK

SEQ ID NO: 229 - protein, 1159 aa, synthetic construct; identical to SEQ ID NO: 19
  REGION 1..1159: note = Description of Artificial Sequence: Synthetic polypeptide

SEQ ID NO: 230 - protein, 1134 aa, synthetic construct
  REGION 1..1134: note = Description of Artificial Sequence: Synthetic polypeptide
MNELVKNRCKQTKTICQKLIPIGKTRETIEKYNLMEIDRKIAANKELMNKLFSLIAGKHINDTLSKCTDLDFEPLLTSLSSLNNAKENDRDNLREYYDSVFEEKKTLAEEISSRLTAVKFAGKDFFTKNIPDFLETYEGDDKNEMSELVSLVIENTVTAGYVKKLEKIDRSMEYRLVSGTVVKRVLTDNADIYEKNIEKAKDFDYGVLNIDEASQFTTLVAKDYANYLTADGIAIYNVGIGKINLALNEYCQKNKEYSYNKLALLPLQKMLYGEKLSLFEKLEDFTSDEELINSYNKFAKTVNESGLAEIIKKAVPSYDEIVIKPNKISNYSNSITGHWSLVNRIMKDYLENNGIKNADKYMEKGLTLSEIGDALENKNIKHSDFISNLINDLGHTYTEIKENKESLKKDESVNALIIKKELDMLLSILQNLKVFDIDNEMFDTGFGIEVSKAIEILGYGVPLYNKIRNYITKKPDPKKKFMTKFGSATIGTGITTSVEGSKKATFLKDGDAVFLLLYNTAGCKANNVSVSNLADLINSSLEIENSGKCYQKMIYQTPGDIKKQIPRVFVYKSEDDDLIKDFKAGLHKTDLSFLNGRLIPYLKEAFATHETYKNYTFSYRNSYESYDEFCEHMSEQAYILEWKWIDKKLIDDLVEDGSLLMFRVWNRFMKKKEGKISKHAKIVNELFSDENASNAAIKLLSVFDIFYRDKQIDNPIVHKAGTTLYNKRTKDGEVIVDYTTMVKNKEKRPNVYTTTKKYDIIKDRRYTEEQFEIHLHVNIGKEENKEKLETSKVINEKKNTLVVTRSNEHLLYVVIFDENDNILLKKSLNTVKGMNFKSKLEVVEIQKKENMQSWKTVGSNQALMEGYLSFAIKEIADLVKEYDAILVLEQNSVGKNILNERVYTRFKEMLITNLSLDVDYENKDFYSYTELGGKVASWRDCVTNGICIQVPSAYKYKDPTTSFSTISMYAKTTAEKSKKLKQIKSFKYNRERGLFELVIAKGVGLENNIVCDSFGSRSIIENDISKEVSCTLKIEKYLIDAGIEYNDEKEVLKDLDTAAKTDAVHKAVTLLLKCFNESPDGRYYISPCGEHFTLCDAPEVLSAINYYIRSRYIREQIVEGVKKMEYKKTILLAK

SEQ ID NO: 231 - protein, 1282 aa, synthetic construct
  REGION 1..1282: note = Description of Artificial Sequence: Synthetic polypeptide
MNGNRIIVYREFVGVTPVAKTLRNELRPIGHTQEHIIHNGLIQEDELRQEKSTELKNIMDDYYREYIDKSLSGVTDLDFTLLFELMNLVQSSPSKDNKKALEKEQSKMREQICTHMQSDSNYKNIFNAKFLKEILPDFIKNYNQYDAKDKAGKLETLALFNGFSTYFTDFFEKRKNVFTKEAVSTSIAYRIVHENSLTFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKENDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMSKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 232 - protein, 1399 aa, synthetic construct
  REGION 1..1399: note = Description of Artificial Sequence: Synthetic polypeptide
MNKDIRKNFTDFVGISEIQKTLRFILIPIGKTAQNIDKYNMFEDDEIRHEYYPILKEACDDFYRNHIDQQFENLELDWSKLDEALASEDRDLINETRATYRQVLFNRLKNSVDIKGDSKKNKTLSLESSDKNLGKKKTKNTFQYNFNDLFKAKLIKAILPLYIEYIYEGEKLENAKKALKMYNRFTSRLSNFWQARANIFTDDEISTGSPYRLVNDNFTIFRINNSIYTKNKPFIEEDILEFEKKLKSKKIIKDFESVDDYFTVNAFNKLCTQNGIDKYNSILGGFTTKEREKVKGLNELFNLAQQSINKGKKGEYRKNIRLGKLTKLKKQILAISDSTSFLIEQIEDDQDLYNKIKDFFELLLKEEIENENIFTQYANLQKLIEQADLSKIYINAKHLNKISHQVTGKWDSLNKGIALLLENININEESKEKSEVISNGQTKDISSEAYKRYLQIQSEEKDIERLRTQIYFSLEDLEKALDLVLIDENMDRSDKSILSYVQSPDLNVNFERDLTDLYSRIMKLEENNEKLLANHSAIDLIKEFLDLIMLRYSRWQILFCDSNYELDQTFYPIYDAVMEILSNIIRLYNLARNYLSRKPDRMKKKKINFNNPTLADGWSESKIPDNSSMLFIKDGMYYLGIIKNRAAYSELLEAESLQSSEKKKSENSSYERMNYHFLPDAFRSIPKSSIAMKAVKEHFEINQKTADLLLDTDKFSKPLRITKEIFDMQYVDLHKNKKKYQVDYLRDTGDKKGYRKALNTWLNFCKDFISKYKGRNLFDYSKIKDADHYETVNEFYNDVDKYSYHIFFTSVAETTVEKFISEGKLYLFQLYNKDFSPHSTGKPNLHTIYWRALFSEENLTSKNIKLNGQAEIFFRPKQIETPFTHKKGSILVNRFDVNGNPIPINVYQEIKGFKNNVIKWDDLNKTTQEGLENDQYLYFESEFEIIKDRRYTEDQLFFHVPISFNWDIGSNPKINDLATQYIVNSNDIHIIGIDRGENHLIYYSVIDLQGAIVEQGSLNTITEYTENKFLNNKTNNLRKIPYKDILQQREDERADARIKWHAIDKIKDLKDGYLGQIVHFLAKLIIKYNAIVILEDLNYGFKRGRFKVERQVYQKFEMALMKKLNVLVFKDYDIDEIGGPLKPWQLTRPIDSYERMGRQNGILFYVPAAYTSAVDPVTGFANLFYLNNVKNSEKFHFFSKFESIKYHSDQDMFSFAFDYNNFGTTTRINDLSKSKWQVFTNHERSVWNNKEKNYVTQNLTDLIKKLLRTYNIEFKNNQNVLDSILKIENNTDKENFARELFRLFRLTIQLRNTTVNENNTEITENELDYIISPVKDKNGNFFDSRDELKNLPDNGDANGAYNIARKGLLYIEQLQESIKTGKLPTLSISTLDWFNYIMK

SEQ ID NO: 233 - protein, 1313 aa, synthetic construct
  REGION 1..1313: note = Description of Artificial Sequence: Synthetic polypeptide
MNKGGYVIMEKMTEKNRWENQFRITKTIKEEIIPTGYTKVNLQRVNMLKREMERNEDLKKMKEICDEYYRNMIDVSLRLEQVRTLGWESLIHKYRMLNKDEKEIKALEKEQEDLRKKISKGFGEKKAWTGEQFIKKILPQYLMDHYTGEELEEKLRIVKKFKGCTMFLSTFFKNRENIFTDKPIHTAVGHRITSENAMLFAANINTYEKMESNVTLEIERLQREFWRRGINISEIFTDAYYVNVLTQKQIEAYNKICGDINQHMNEYCQKQKLKFSEFRMRELKKQILAVVGEHFEIPEKIESTKEVYRELNEYYESLKELHGQFEELKSVQLKYSQIYVQKKGYDRISRYIGGQWDLIQECMKKDCASGMKGTKKNHDAKIEEEVAKVKYQSIEHIQKLVCTYEEDRGHKVTDYVDEFIVSVCDLLGADHIITRDGERIELPLQYEPGTDLLKNDTINQRRLSDIKTILDWHMDMLEWLKTFLVNDLVIKDEEFYMAIEELNERMQCVISVYNRIRNYVTQKGYEPEKIRICFDKGTILTGWTTGDNYQYSGFLLMRNDKYYLGIINTNEKSVRKILDGNEECKDENDYIRVGYHLINDASKQLPRIFVMPKAGKKSEILMKDEQCDYIWDGYCHNKHNESKEFMRELIDYYKRSIMNYDKWEGYCFKFSSTESYDNMQDFYKEVREQSYNISFSYINENVLEQLDKDGKIYLFQVYNKDFAAGSTGTPNLHTMYLQNLFSSQNLELKRLRLGGNAELFYRPGTEKDVTHRKGSILVDRTYVREEKDGIEVRDTVPEKEYLEIYRYLNGKQKGDLSESAKQWLDKVHYREAPCDIIKDKRYAQEKYFLHFSVEINPNAEGQTALNDNVRRWLSEEEDIHVIGIDRGERNLIYVSLMDGKGRIKDQKSYNIVNSGNKEPVDYLAKLKVREKERDEARRNWKAIGKIKDIKTGYLSYVVHEIVEMAVREKAIIVMEDLNYGFKRGRFKVERQVYQKFEEMLINKLNYVVDKQLSVDEPGGLLRGYQLAFIPKDKKSSMRQNGIVFYVPAGYTSKIDPTTGFVNIFKFPQFGKGDDDGNGKDYDKIRAFFGKFDEIRYECDEKVTADNTREVKERYRFDFDYSKFETHLVHMKKTKWTVYAEGERIKRKKVGNYWTSEVISDIALRMSNTLNIAGIEYKDGHNLVNEICALRGKQAGIILNELLEIVRLTVQLRNSTTEGDVDERDEIISPVLNEKYGCFYHSTEYKQQNGDVLPKDADANGAYCIGLKGIYEIRQIKNKWKEDMTKGEGKALNEGMRISHDQWFEFIQNMNKGE

SEQ ID NO: 234 - protein, 1470 aa, synthetic construct
  REGION 1..1470: note = Description of Artificial Sequence: Synthetic polypeptide
MNNPRGAFGGFTNLYSLSKTLRFELKPYLEIPEGEKGKLFGDDKEYYKNCKTYTEYYLKKANKEYYDNEKVKNTDLQLVNFLHDERIEDAYQVLKPVFDTLHEEFITDSLESAEAKKIDFGNYYGLYEKQKSEQNKDEKKKIDKPLETERGKLRKAFTPIYEAEGKNLKNKAGKEKKDKDILKESGFKVLIEAGILKYIKNNIDEFADKKLKNNEGKEITKKDIETALGAENIEGIFDGFFTYFSGFNQNRENYYSTEEKATAVASRIVDENLSKFCDNILLYRKNENDYLKIFNFLKNKGKDLKLKNSKFGKENEPEFIPAYDMKNDEKSFSVADFVNCLSQGEIEKYNAKIANANYLINLYNQNKDGNSSKLSMFKILYKQIGCGEKKDFIKTIKDNAELKQILEKACEAGKKYFIRGKSEDGGVSNIFDFTDYIQSHENYKGVYWSDKAINTISGKYFANWDTLKNKLGDAKVFNKNTGEDKADVKYKVPQAVMLSELFAVLDDNAGEDWREKGIFFKASLFEGDQNKSEIIKNANRPSQALLKMICDDMESLAKNFIDSGDKILKISDRDYQKDENKQKIKNWLDNALWINQILKYFKVKANKIKGDSIDARIDSGLDMLVFSSDNPAEDYDMIRNYLTQKPQDEINKLKLNFENSSLAGGWDENKEKDNSCIILKDEQDKQYLAVMKYENTKVFEQKNSQLYIADNAAWKKMIYKLVPGASKTLPKVFFSKKWTANRPTPSDIVEIYQKGSFKKENVDFNDKKEKDESRKEKNREKIIAELQKTCWMDIRYNIDGKIESAKYVNKEKLAKLIDFYKENLKKYPSEEESWDRLFAFGFSDTKSYKSIDQFYIEVDKQGYKLEFVTINKARLDEYVRDGKIYLFEIRSRDNNLVNGEEKTSAKNLQTIYWNAAFGGDDNKPKLNGEAEIFYRPAIAENKLNKKKDKNGKEIIDGYRFSKEKFIFHCPITLNFCLKETKINDKLNAALAKPENGQGVYFLGIDRGEKHLAYYSLVNQKGEILEQGTLNLPFLDKNGKSRSIKVEKKSFEKDSNGGIIKDKDGNDKIKIEFVECWNYNDLLDARAGDRDYARKNWTTIGTIKELKDGYISQVVRKIVDLSIYKNTETKEFREMPAFIVLEDLNIGFKRGRQKIEKQVYQKLELALAKKLNFLVDKKADIGEIGSVTKAIQLTPPVNNFGDMENRKQFGNMLYIRADYTSQTDPATGWRKSIYLKSGSESNVKEQIEKSFFDIRYESGDYCFEYRDRHGKMWQLYSSKNGVSLDRFHGERNNSKNVWESEKQPLNEMLDILFDEKRFDKSKSLYEQMFKGGVALTRLPKEINKKDKPAWESLRFVIILIQQIRNTGKNGDDRNGDFIQSPVRDEKTGEHFDSRIYLDKEQKGEKADLPTSGDANGAYNIARKGIVVAEHIKRGFDKLYISDEEWDTWLAGDEIWDKWLKENRESLTKTRK

SEQ ID NO: 235 - protein, 1259 aa, synthetic construct
  REGION 1..1259: note = Description of Artificial Sequence: Synthetic polypeptide
MNYKTGLEDFIGKESLSKTLRNALIPTESTKIHMEEMGVIRDDELRAEKQQELKEIMDDYYRTFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYSVDFYDRELTQPGIEYYNGICGKINEHMNQFCQKNRINKNDFRMKKLHKQILCKKSSYYEIPFRFESDQEVYDALNEFIKTMKKKEIIRRCVHLGQECDDYDLGKIYISSNKYEQISNALYGSWDTIRKCIKEEYMDALPGKGEKKEEKAEAAAKKEEYRSIADIDKIISLYGSEMDRTISAKKCITEICDMAGQISIDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPIVHKKGSVLVNRSYTQTVGNKEIRVSIPEEYYTEIYNYLNHIGKGKLSSEAQRYLDEGKIKSFTATKDIVKNYRYCCDHYFLHLPITINFKAKSDVAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTILASTKWKVYTNGTRLKKIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 236 - protein, 477 aa, synthetic construct
  REGION 1..477: note = Description of Artificial Sequence: Synthetic polypeptide
MRISKTLSLRIVRPFYTPEVEAGIKAEKDKREAQGQTRSLDAKFFNELKKKHSEIILSSEFYSLLSEVQRQLTSIYNHAMSNLYHKIIVEGEKTSTSKALSNIGYDECKAIFPSYMALGLRQKIQSNFRRRDLKNFRMAVPTAKSDKFPIPIYRQVDGSKGGFKISENDGKDFIVELPLVDYVAEEVKTAKGRFTKINISKPPKIKNIPVILSTLRRRQSGQWFSDDGTNAEIRRVISGEYKVSWIEIVRRTRFGKHDDWFVNMVIKYDKPEEGLDSKVVGGIDVGVSSPLVCALNNSLDRYFVKSSDIIAFNKRAMARRRTLLRQNKYKRSGHGSKNKLEPITVLTEKNERFKKSIMQRWAKEVAEFFRGKGASVVRMEELSGLKEKDNFFSSYLRMYWNYGQLQQIIENKLKEYGIKVNYVSPKDTSKKCHSCTHINEFFTFEYRQKNNFPLFKCEKCGVECSADYNAAKNMAIA

SEQ ID NO: 237 - protein, 1293 aa, synthetic construct
  REGION 1..1293: note = Description of Artificial Sequence: Synthetic polypeptide
MRTMVTFEDFTKQYQVSKTLRFELIPQGKTLENMKRDGIISVDRQRNEDYQKAKGILDKLYKYILDFTMETVVIDWEALATATEEFRKSKDKKTYEKVQSKIRTALLEHVKKQKVGTEDLFKGMFSSKIITGEVLAAFPEIRLSDEENLILEKFKDFTTYFTGFFENRKNVFTDEALSTSFTYRLVNDNFIKFFDNCIVFKNVVNISPHMAKSLETCASDLGIFPGVSLEEVFSISFYNRLLTQTGIDQFNQLLGGISGKEGEHKQQGLNEIINLAMQQNLEVKEVLKNKAHRFTPLFKQILSDRSTMSFIPDAFADDDEVLSAVDAYRKYLSEKNIGDRAFQLISDMEAYSPELMRIGGKYVSVLSQLLFYSWSEIRDGVKAYKESLITGKKTKKELENIDKEIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAILLRKEGEYFLGIFNSKNKPKLVSDGGAGIGYEKMIYKQFPDFKKMLPKCTISLKDTKAHFQKSDEDFTLQTDKFEKSIVITKQIYDLGTQTVNGKKKFQVDYPRLTGDMEGYRAALKEWIDFGKEFIQAYTSTAIYDTSLFRDSSDYPDLPSFYKDVDNICYKLTFEWIPDAVIDDCIDDGSLYLFKLHNKDFSSGSIGKPNLHTLYWKALFEEENLSDVVVKLNGQAELFYRPKSLTRPVVHEEGEVIINKTTSTGLPVPDDVYVELSKFVRNGKKGNLTDKAKNWLDKVTVRKMPHAITKDRRFTVDKFFFHVPITLNYKADSSPYRFNDFVRQYIKDCSDVKIIGIDRGERNLIYAVVIDGKGNIIEQRSFNTVGTYNYQEKLEQKEKERQTARQDWATVTKIKDLKKGYLSAVVHELSKMIVKYKAIVALENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPYDADANGAFHIAMKGLLLTERIRNDDKLAISNEEWLNYIQEMRG

SEQ ID NO: 238 - protein, 1228 aa, synthetic construct; identical to SEQ ID NO: 36
  REGION 1..1228: note = Description of Artificial Sequence: Synthetic polypeptide

SEQ ID NO: 239 - protein, 1286 aa, synthetic construct
  REGION 1..1286: note = Description of Artificial Sequence: Synthetic polypeptide
MTNFDNFTKKYVNSKTIRLEAIPVGKTLKNIEKMGFIAADRQRDEDYQKAKSVIDHIYKAFMDDCLKDLFLDWDPLYEAVVACWRERSPEGRQALQIMQADYRKKIADRFRNHELYGSLFTKKIFDGSVAQRLPDLEQSAEEKSLLSNFNKFTSYFRDFFDKRKRLFSDDEKHSAIAYRLINENFLKFVANCEAFRRMTERVPELREKLQNTGSLQVYNGLALDEVFSADFYNQLIVQKQIDLYNQLIGGIAGEPGTPNIQGLNATINLALQGDSSLHEKLAGIPHRFNPLYKQILSDVSTLSFVPSAFQSDGEMLAAVRGFKVQLESGRVLQNVRRLFNGLETEADLSRVYVNNSKLAAFSSMFFGRWNLCSDALFAWKKGKQKKITNKKLTEIKKWLKNSDIAIAEIQEAFGEDFPRGKINEKIQAQADALHSQLALPIPENLKALCAKDGLKSMLDTVLGLYRMLQWFIVGDDNEKDSDFYFGLGKILGSLDPVLVLYNRVRNYITKKPYSLTKFRLNFDNSQLLNGWDENNLDTNCASIFIKDGKYYLGISNKNNRPQFDTVATSGKSGYQRMVYKQFANWGRDLPHSTTQMKKVKKHFSASDADYVLDGDKFIRPLIITKEIFDLNNVKFNGKKKLQVDYLRNTGDREGYTHALHTWINFAKDFCACYKSTSIYDISCLRPTDQYDNLMDFYADLGNLSHRIVWQTIPEEAIDNYVEQGQLFLFQLYNKDFAPGADGKPNLHTLYWKAVFNPENLEDVVVKLNGKAELFYRPRSNMDVVRHKVGEKLVNRKLKNGLTLPSRLHEEIYRYVNGTLNKDLSADARSVLPLAVVRDVQHEIIKDRRFTADKFFFHASLTFNFKSSDKPVGFNEDVREYLREHPDTYVVGVDRGERNLIYIVVIDPQGNIVEQRSFNMINGIDYWSLLDQKEKERVEAKQAWETVGKIKDLKCGYLSFLIHEITKIIIKYHAVVILENLSLGFKRVRTGIAEKAVYQQFERMLVTKLGYVVFKDRAGKAPGGVLNAYQLTDNTRTAENTGIQNGFLFYVPAAFTSRVDPATGFFDFYDWGKIKTATDKKNFIAGFNSVRYERSTGDFIVHVGAKNLAVRRVAEDVRTEWDIVIEANVRKMGIDGNSYISGKRIRYRSGEQGHGQYENHLPCQELIRALQQYGIQYETGKDILPAILQQDDAKLTDTVFDVFRLALQMRNTSAETGEDYFNSVVRDRSGRCFDTRRAEAAMPKEADANDAYHIALKGLFVLEKLRKGESIGIKNTEWLRYVQQRHS

SEQ ID NO: 240 - protein, 1272 aa, synthetic construct
  REGION 1..1272: note = Description of Artificial Sequence: Synthetic polypeptide
MTPIFCNFVVYQIMLFNNNININVKTMNKKHLSDFTNLFPVSKTLRFRLEPQGKTMENIVKAQTIETDEERSHDYEKTKEYIDDYHRQFIDDTLDKFAFKVESTGNNDSLQDYLDAYLSANDNRTKQTEEIQTNLRKAIVSAFKMQPQFNLLFKKEMVKHLLPQFVDTDDKKRIVAKFNDFTTYFTGFFTNRENMYSDEAKSTSIAYRIVNQNLIKFVENMLTFKSHILPILPQEQLATLYDDFKEYLNVASIAEMFELDHFSIVLTQRQIEVYNSVIGGRKDENNKQIKPGLNQYINQHNQAVKDKSARLPLLKPLFNQILSEKAGVSFLPKQFKSASEVVKSLNEAYAELSPVLAAIQDVVTNITDYDCNGIFIKNDLGLTDIAQRFYGNYDAVKRGLRNQYELETPMHNGQKAEKYEEQVAKHLKSIESVSLAQINQVVTDGGDICDYFKAFGATDDGDIQRENLLASINNAHTAISPVLNKENANDNELRKNTMLIKDLLDAIKRLQWFAKPLLGAGDETNKDQVFYGKFEPLYNQLDETISPLYDKVRSYLTKKPYSLDKFKINFEKSNLLGGWDPGADRKYQYNAVILRKDNDFYLGIMRDEATSKRKCIQVLDCNDEGLDENFEKVEYKQIKPSQNMPRCAFAKKECEENADIMELKRKKNAKSYNTNKDDKNALIRHYQRYLDRTYPEFGFVYKDADEYDTVKAFTDSMDSQDYKLSFLQVSETGLNKLVDEGDLYLFKITNKDFSSYAKGRPNLHTIYWRMLFDPKNLANVVYKLEGKAEVFFRRKSLASTTTHKAKQAIKNKSRYNEAVKPQSTFDYDIIKDRRFTADKFEFHVPIKMNFKAAGWNSTRLTNEVREFIKSQGVRHIIGIDRGERHLLYLTMIDMDGNIVKQCSLNAPAQDNARASEVDYHQLLDSKEADRLAARRNWGTIENIKELKQGYLSQVVHLLATMMVDNDAILVLENLNAGFMRGRQKVEKSVYQKFEKMLIDKLNYIVDKGQSPDKPTGALHAVQLTGLYSDFNKSNMKRANVRQCGFVFYIPAWNTSKIDPVTGFVNLFDTHLSSMGEIKAFFSKFDSIRYNQDKGWFEFKFDYSRFTTRAEGCRTQWTVCTYGERIWTHRSKNQNNQFVNDTVNVTQQMLQLLQDCGIDPNGNLKEAIANIDSKKSLETLLHLFKLTVQMRNSVTGSEVDYMISPVADERGHFFDSRESDEHLPANADANGAFNIARKGLMVVRQIMATDDVSKIKFAVSNKDWLRFAQHIDD

SEQ ID NO: 241 - protein, 477 aa, synthetic construct
  REGION 1..477: note = Description of Artificial Sequence: Synthetic polypeptide
VKISKTLSLRIIRPYYTPEVESAIKAEKDKREAQGQTRNLDAKFFNELKKKHPQIILSGEFYSLLFEMQRQLTSIYNRAMSSLYHKIIVEGEKTSTSKALSDIGYDECKSVFPSYIALGLRQKIQSNFRRKELKGFRMAVPTAKSDKFPIPIYKQVDDGKGGFKISENKEGDFIVELPLVEYTAEDVKTAKGKFTKINISKPPKIKNIPVILSTLRRKQSGQWFSDEGTNAEIRRVISGEYKVSWIEVVRRTRFGKHDDWFLNIVIKYDKTEDGLDPEVVGGIDVGVSTPLVCAVNNSLDRYFVKSSDIIAFKKRAMARRRTLLRQNRFKRSGHGSKSKLEPITILTEKNERFKKSIMQRWAKEVAEFFKGERASVVQMEELSGLKEKDNFFGSYLRMYWNYGQLQQIIENKLKEYGIKVNYVSPKDTSKKCHSCGYINEFFTFEFRQKNNFPLFKCKKCGVECNADYNAAKNIAIA

SEQ ID NO: 242 - protein, 992 aa, synthetic construct
  REGION 1..992: note = Description of Artificial Sequence: Synthetic polypeptide
VKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 243 - DNA, 33 bp, synthetic construct
  misc_feature 1..33: note = Description of Artificial Sequence: Synthetic oligonucleotide
  misc_feature 1..33: note = Description of Combined DNA/RNA Molecule: Synthetic oligonucleotide
  source 1..33: mol_type = other DNA
  misc_feature 15..19: note = RNA
  misc_feature 1..14: note = DNA
  misc_feature 20..33: note = DNA
tacaacgactagcatttttcaggtaaggctagc

(19) United States
(12) Patent Application Publication
(10) Pub. No.: US 2024/0102084 A1
(43) Pub. Date: Mar. 28, 2024

(54) COMPOSITIONS AND METHODS FOR DETECTION OF A NUCLEIC ACID

(71) Applicant: Mammoth Biosciences, Inc., Brisbane, CA (US)

(72) Inventors: James Paul BROUGHTON, South San Francisco, CA (US); Janice Sha CHEN, San Francisco, CA (US)

(21) Appl. No.: 18/185,314

(22) Filed: Mar. 16, 2023

Related U.S. Application Data
(63) Continuation of application No. PCT/US21/50952, filed on Sep. 17, 2021.
(60) Provisional application No. 63/079,965, filed on Sep. 17, 2020.

(51) Int. Cl. C12Q 1/6823; C12N 9/22; C12Q 1/44

(52) U.S. Cl. CPC C12Q 1/6823; C12N 9/22; C12Q 1/44; C12N 2310/20

(57) ABSTRACT

Described herein are methods, devices, systems, and compositions for detecting a target nucleic acid using a programmable nuclease and a signal amplifier. Such methods, devices, systems, and compositions can comprise using signal amplifiers, such as catalytic oligonucleotides, which can be activated by programmable nucleases and be configured to cleave a reporter molecule upon activation. Signals can be generated and detected upon cleavage of reporter molecules.

### CROSS REREFERENCE

This application claims the benefit of U.S. Provisional Patent Application No. 63/079,965, filed on Sep. 17, 2020, which is hereby incorporated by reference in its entirety.

### SEQUENCE LISTING

The instant application contains a Sequence Listing which has been submitted electronically in XML format and is hereby incorporated by reference in its entirety. Said XML copy, created on Dec. 19, 2023, is named 203477-736301_SL.xml and is 471,708 bytes in size.

### BACKGROUND

The detection of target nucleic acids in a sample can provide valuable information about the sample. For example, detection of a target nucleic acid provides guidance on treatment or intervention to reduce the progression or transmission of an ailment that is associated with or results from the target nucleic acid. Often, the target nucleic acid can be in a low concentration in a sample. There exists a need for systems that can rapidly and accurately detect target nucleic acids in a sample, especially low concentrations of target nucleic acids in a sample.

### SUMMARY

Described herein are compositions, systems, devices, and methods for detection of target nucleic acids. Often, the compositions as described herein are used in methods and/or in systems or devices for detecting a low concentration of nucleic acids in a sample. A composition, system, device, and/or method of use thereof as described herein can comprise a guide nucleic acid that binds to a target nucleic acid, a programmable nuclease, a signal amplifier, which can be activated upon binding of the programmable nuclease to the target nucleic acid, and reporter molecules. In some embodiments, the signal amplifier can a comprise an enzyme, which can be activated (e.g., unbound, released, etc.) upon activation of the programmable nuclease by binding to the target nucleic acid. In some embodiments, the signal amplifier can comprise a catalytic oligonucleotide, which can be cleaved and activated by the programmable nuclease upon activation of the programmable nuclease by binding to the target nucleic acid. In some examples, the catalytic oligonucleotide can comprise a DNAzyme that is activated upon cleavage of the catalytic oligonucleotide by the programmable nuclease. In some examples, the catalytic oligonucleotide molecule can comprise a ribozyme that is activated upon cleavage of the catalytic oligonucleotide by the programmable nuclease. After cleavage by the programmable nuclease, the catalytic oligonucleotide can cleave a reporter molecule, thereby generating a signal that can be detected and assayed. The signal resulting from the compositions described herein can be amplified compared to a signal generated from a composition as described herein, but which lacks a catalytic oligonucleotide.

Described herein, in certain embodiments, is a composition comprising a signal amplifier, a programmable nuclease, and a guide nucleic acid that hybridizes to a segment of a target nucleic acid. In some embodiments, the signal amplifier is a catalytic oligonucleotide. In some embodiments, the catalytic oligonucleotide has a circular structure. In some embodiments, the catalytic oligonucleotide comprises a programmable nuclease cleavage site. In some embodiments, the catalytic oligonucleotide is configured to cleave a nucleotide molecule upon cleavage by the programmable nuclease. In some embodiments, the composition further comprises a blocker oligonucleotide. In some embodiments, the catalytic oligonucleotide is bound to the blocker oligonucleotide. In some embodiments, the blocker oligonucleotide comprises ribonucleotides, deoxyribonucleotides, or a combination thereof. In some embodiments, the blocker oligonucleotide comprises a programmable nuclease cleavage site, a catalytic oligonucleotide recognition site, or a combination thereof. In some embodiments, the catalytic oligonucleotide is configured to cleave a nucleotide molecule upon cleavage of the blocker oligonucleotide by the programmable nuclease. In some embodiments, the catalytic oligonucleotide comprises an enzyme. In some embodiments, the catalytic oligonucleotide comprises a DNAzyme. In some embodiments, the catalytic oligonucleotide comprises a ribozyme. In some embodiments, the catalytic oligonucleotide comprises deoxyribonucleotides. In some embodiments, the catalytic oligonucleotide comprises ribonucleotides. In some embodiments, the programmable nuclease comprises a HEPN cleaving domain. In some embodiments, the programmable nuclease is a type VI CRISPR/Cas effector protein. In some embodiments, the type VI CRISPR/Cas effector protein is a Cas13 protein. In some embodiments, the Cas13 protein comprises a Cas13a polypeptide, a Cas13b polypeptide, a Cas13c polypeptide, a Cas13c polypeptide, a Cas13d polypeptide, or a Cas13e polypeptide. In some embodiments, the programmable nuclease comprises a RuvC catalytic domain. In some embodiments, the programmable nuclease is a type V CRISPR/Cas effector protein. In some embodiments, the type V CRISPR/Cas effector protein is a Cas12 protein. In some embodiments, the Cas12 protein comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide. In some embodiments, the type V CRIPSR/Cas effector protein is a Cas14 protein. In some embodiments, the Cas14 protein comprises a Cas14a polypeptide, a Cas14b polypeptide, a Cas14c polypeptide, a Cas14d polypeptide, a Cas14e polypeptide, a Cas 14f polypeptide, a Cas14g polypeptide, a Cas14h polypeptide, a Cas14i polypeptide, a Cas14j polypeptide, or a Cas14k polypeptide. In some embodiments, the type V CRIPSR/Cas effector protein is a Case protein. In some embodiments, the composition further comprises the target nucleic acid. In some embodiments, the target nucleic acid is a target RNA. In some embodiments, the target nucleic acid is a target DNA. In some embodiments, the target nucleic acid is an amplicon. In some embodiments, the composition further comprises a reporter molecule. In some embodiments, the reporter molecule is configured to generate a signal upon cleavage by the catalytic oligonucleotide, the programmable nuclease, or both. In some embodiments, the reporter molecule comprises single stranded deoxyribonucleic acids, single stranded ribonucleic acids, or single stranded deoxyribonucleic acids and ribonucleic acids. In some embodiments, the reporter molecule comprises a fluorophore and a quencher moiety. In some embodiments, the programmable nuclease is a first programmable nuclease and the composition further comprises a second programmable nuclease.

Described herein, in certain embodiments, is a composition comprising a first signal amplifier, a second signal amplifier, a programmable nuclease, and a guide nucleic acid that hybridizes to a segment of a target nucleic acid. In some embodiments, the first signal amplifier is a first catalytic oligonucleotide. In some embodiments, the second signal amplifier is a second catalytic oligonucleotide. In some embodiments, the first catalytic oligonucleotide is configured to cleave a nucleotide molecule upon cleavage by the programmable nuclease. In some embodiments, the composition further comprises a first blocker oligonucleotide and a second blocker oligonucleotide. In some embodiments, the first blocker oligonucleotide is bound to the first catalytic oligonucleotide and the second blocker oligonucleotide is bound to the second catalytic oligonucleotide. In some embodiments, the first blocker oligonucleotide comprises ribonucleotides, deoxyribonucleotides, or a combination thereof. In some embodiments, the second blocker oligonucleotide comprises ribonucleotides, deoxyribonucleotides, or a combination thereof. In some embodiments, the first blocker oligonucleotide comprises a programmable nuclease cleavage site and a second catalytic oligonucleotide recognition site and the second blocker oligonucleotide comprises a first catalytic oligonucleotide recognition site. In some embodiments, the first catalytic oligonucleotide is configured to cleave a nucleotide molecule upon cleavage of the first blocker oligonucleotide by the programmable nuclease or upon cleavage by the second catalytic oligonucleotide. In some embodiments, the first catalytic oligonucleotide comprises a first enzyme and the second catalytic oligonucleotide comprises a second enzyme. In some embodiments, the first catalytic oligonucleotide comprises a DNAzyme. In some embodiments, the second catalytic oligonucleotide comprises a DNAzyme. In some embodiments, the first catalytic oligonucleotide comprises a ribozyme. In some embodiments, the second catalytic oligonucleotide comprises a ribozyme. In some embodiments, the first catalytic oligonucleotide comprises deoxyribonucleotides. In some embodiments, the second catalytic oligonucleotide comprises deoxyribonucleotides. In some embodiments, the first catalytic oligonucleotide comprises ribonucleotides. In some embodiments, the second catalytic oligonucleotide comprises ribonucleotides. In some embodiments, the programmable nuclease comprises a HEPN cleaving domain. In some embodiments, the programmable nuclease is a type VI CRISPR/Cas effector protein. In some embodiments, the type VI CRISPR/Cas effector protein is a Cas13 protein. In some embodiments, the Cas13 protein comprises a Cas13a polypeptide, a Cas13b polypeptide, a Cas13c polypeptide, a Cas13c polypeptide, a Cas13d polypeptide, or a Cas13e polypeptide. In some embodiments, the programmable nuclease comprises a RuvC catalytic domain. In some embodiments, the programmable nuclease is a type V CRISPR/Cas effector protein. In some embodiments, the type V CRISPR/Cas effector protein is a Cas12 protein. In some embodiments, the Cas12 protein comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide. In some embodiments, the type V CRIPSR/Cas effector protein is a Cas14 protein. In some embodiments, the Cas14 protein comprises a Cas14a polypeptide, a Cas14b polypeptide, a Cas14c polypeptide, a Cas14d polypeptide, a Cas14e polypeptide, a Cas14f polypeptide, a Cas14g polypeptide, a Cas14h polypeptide, a Cas14i polypeptide, a Cas14j polypeptide, or a Cas14k polypeptide. In some embodiments, the type V CRIPSR/Cas effector protein is a Case protein. In some embodiments, the composition further comprises the target nucleic acid. In some embodiments, the target nucleic acid is a target RNA. In some embodiments, the target nucleic acid is a target DNA. In some embodiments, the target nucleic acid is an amplicon. In some embodiments, the composition further comprises a reporter molecule. In some embodiments, the reporter molecule is configured to generate a signal upon cleavage by the first catalytic oligonucleotide, the programmable nuclease, or both. In some embodiments, the reporter molecule comprises single stranded deoxyribonucleic acids, single stranded ribonucleic acids, or single stranded deoxyribonucleic acids and ribonucleic acids. In some embodiments, the reporter molecule comprises a fluorophore and a quencher moiety.

Described herein, in certain embodiments, is a method of nucleic acid detection comprising: (a) contacting a sample to a composition comprising a plurality of reporter molecules and any of the compositions described herein; and (b) assaying for a signal produced by and/or indicative of cleavage of the reporter molecule. In some embodiments, the catalytic oligonucleotide is a circular polyribonucleotide before the contacting step. In some embodiments, the blocker oligonucleotide is bound to the catalytic oligonucleotide before the contacting step. In some embodiments, the first catalytic oligonucleotide is bound to the first blocker oligonucleotide and the second catalytic oligonucleotide is bound to the second blocker oligonucleotide before the contacting step. In some embodiments, a reporter molecule of the plurality of reporter molecules comprises a cleavage site for the catalytic oligonucleotide or the first catalytic oligonucleotide. In some embodiments, a reporter molecule of the plurality of reporter molecules comprises a fluorophore and a quencher moiety. In some embodiments, the sample comprises nucleic acids. In some embodiments, the sample comprises the target nucleic acid or an amplicon thereof.

Described herein, in certain embodiments, is a method of nucleic acid detection comprising: (a) contacting a sample comprising a plurality of nucleic acids to a composition comprising a plurality of reporter molecules, a programmable nuclease complex comprising a programmable nuclease coupled to a guide nucleic acid that hybridizes to a segment of a target nucleic acid, and a signal amplifier; (b) when the target nucleic acid is present in the plurality of nucleic acids, activating the programmable nuclease complex by hybridizing the target nucleic acid, or an amplicon thereof, to the guide nucleic acid; (c) activating the signal amplifier with the activated programmable nuclease complex, wherein the activated signal amplifier is configured to cleave at least a reporter molecule of the plurality of reporter molecules; and (d) assaying for a signal produced by or indicative of cleavage of the reporter molecule. In some embodiments, the signal amplifier comprises an enzyme. In some embodiments, the signal amplifier comprises a catalytic oligonucleotide. In some embodiments, the signal amplifier is configured to cleave a same reporter molecule as the programmable nuclease. In some embodiments, the signal amplifier is configured to cleave a different reporter molecule than the programmable nuclease. In some embodiments, the signal is produced by or indicative of cleavage of a same reporter by both the programmable nuclease and the signal amplifier. In some embodiments, the signal is produced by or indicative of cleavage of a first reporter by the programmable nuclease, a second reporter by the signal amplifier, or both. In some embodiments, activation of the signal amplifier by the activated programmable nuclease complex may generate a positive feedback loop to generate the signal.

These and other embodiments are described in further detail in the following description related to the appended drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a schematic of an exemplary method of signal amplification using a composition comprising a catalytic oligonucleotide, a programmable nuclease, a guide nucleic acid, a target nucleic acid molecule, and a reporter molecule, in accordance with embodiments.

FIG. 2 shows a schematic of an exemplary method of signal amplification using a composition comprising a catalytic oligonucleotide, a blocker oligonucleotide, a programmable nuclease, a guide nucleic acid, a target nucleic acid molecule, and a reporter molecule, in accordance with embodiments.

FIG. 3A shows a schematic of activation of a catalytic oligonucleotide (310) in a catalytic oligonucleotide/blocker oligonucleotide complex (301) by cleavage of a programmable nuclease cleavage site (314) on a blocker oligonucleotide (312) and subsequent binding of the catalytic oligonucleotide (317) to a reporter molecule (318) for cleavage of the reporter molecule, in accordance with embodiments.

FIG. 3B shows a schematic of activation of a catalytic oligonucleotide (310) in a catalytic oligonucleotide/blocker oligonucleotide complex (302) by cleavage of a programmable nuclease cleavage site (314) on the blocker oligonucleotide (312), and the subsequent multi-functional capacity of the catalytic oligonucleotide (317) to bind to a reporter molecule (318) for cleavage of the reporter molecule and/or bind to another catalytic oligonucleotide/blocker oligonucleotide complex (303) for cleavage of a catalytic oligonucleotide recognition site (316) on the blocker oligonucleotide for activation of another catalytic oligonucleotide, in accordance with embodiments.

FIG. 4 shows a schematic of activation of a first catalytic oligonucleotide (410) in a first catalytic oligonucleotide/blocker oligonucleotide complex (401) by cleavage of a programmable nuclease cleavage site (414) on the blocker oligonucleotide (412), and the subsequent multi-functional capacity of the first catalytic oligonucleotide (417) to bind to a reporter molecule (418) for cleavage of the reporter molecule and/or bind to a second catalytic oligonucleotide/blocker oligonucleotide complex (402) for cleavage of a first catalytic oligonucleotide recognition site (424) on the second blocker oligonucleotide (422) for activation of the second catalytic oligonucleotide (420). The activated second catalytic oligonucleotide (426) can subsequently bind to and cleave a second catalytic oligonucleotide recognition site (416) on another first catalytic oligonucleotide/blocker oligonucleotide complex (403) for activation of another first catalytic oligonucleotide (410), in accordance with embodiments.

FIG. 5 shows a fluorometric assay comparison of a Cas13 protein cleavage efficiency of a reporter molecule optimized for cleavage by the Cas13 protein (520) and a reporter molecule optimized for cleavage by a catalytic oligonucleotide (DNAzyme) (510) in the presence various concentrations of target nucleic acids.

FIG. 6A shows fluorometric assays of a Cas13 protein cleavage efficiency of reporter molecules in CutSmart Buffer with various concentrations of MgCl₂ and in the presence of 40 nM Cas13 and 1.25 pM or 0 pM of target RNA.

FIG. 6B shows fluorometric assays of a Cas13 protein cleavage efficiency of reporter molecules in MBuffer1 with various concentrations of MgCl₂ and in the presence of 40 nM Cas13 and 1.25 or 0 pM target RNA.

FIG. 7A shows fluorometric assays of a catalytic oligonucleotide (DNAzyme; DZ-act-linear) cleavage efficiency of reporter molecules in CutSmart Buffer with various concentrations of MgCl₂ and in the presence of 50 nM catalytic oligonucleotide or 1 nM catalytic oligonucleotide.

FIG. 7B shows fluorometric assays of a catalytic oligonucleotide (DNAzyme; DZ-act-linear) cleavage efficiency of reporter molecules in MBuffer1 with various concentrations of MgCl₂ and in the presence of 50 nM catalytic oligonucleotide or 1 nM catalytic oligonucleotide.

FIG. 8 shows fluorometric assays of a catalytic oligonucleotide (DNAzyme) cleavage efficiency of reporter molecules in the presence of various concentrations of catalytic oligonucleotide:blocker oligonucleotide ratios. Each ratio was tested with the catalytic oligonucleotide alone (DNAzyme) or with the catalytic oligonucleotide and programmable nuclease (Cas13+DNAzyme).

FIG. 9 shows fluorometric assays of a catalytic oligonucleotide (DNAzyme) cleavage efficiency of reporter molecules in the presence of various concentrations of catalytic oligonucleotide:blocker oligonucleotide ratios. Each ratio was tested with the catalytic oligonucleotide alone (DNAzyme) or with the catalytic oligonucleotide and programmable nuclease (Cas13+DNAzyme).

FIG. 10 shows fluorometric assays of cleavage efficiency of reporter molecules with either 0 pM target nucleic acid or 50 pM target nucleic acid, reporter molecules (rep091), and in the presence of a Cas13 protein (Cas13 alone; 1100), a Cas13 protein and a catalytic oligonucleotide (Cas 13+DNAzyme; 1110), or a catalytic oligonucleotide (DNAzyme alone; 1120).

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or portions of the method or process may be performed in any suitable sequence and are not necessarily limited to any particular disclosed sequence. Various operations may be described as multiple discrete operations in turn, in a manner that may be helpful to understanding certain embodiments, however, the order of the description should not be construed to imply that these operations are order dependent. Additionally, structures, systems, and/or devices described herein may be embodied as integrated components or as separate components.

For the purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages taught herein without necessarily achieving other aspects or advantages as may also be taught or suggested herein.

The capability to quickly and accurately detect the presence or absence of a target nucleic acid can provide valuable information associated with the presence of the target nucleic acid in a sample. For example, the capability to quickly and accurately detect the presence of a target nucleic acid associated with, or causing, an ailment in a subject can provide valuable information and may lead to actions taken to reduce the progression or transmission of the ailment in response. There exists a need for systems that can detect target nucleic acids in a sample, especially when said target nucleic acids exist in low concentrations in a sample. It would also be desirable to provide compositions and methods which enable detection of target nucleic acids with or without nucleic acid amplification prior to or concurrent with detection. In some embodiments, the target nucleic acid is an amplicon.

The present invention is described in relation to compositions, methods, systems, and devices for performing nucleic acid detection assays using a catalytic oligonucleotide-based signal amplifier (also referred to herein as a signal amplifying moiety or component) in a programmable nuclease-driven manner. However, one of ordinary skill in the art will appreciate that this is not intended to be limiting and the devices and methods disclosed herein may be used in other nucleic acid detection assays or with other signal amplifiers. For example, a signal amplifier may be an enzyme, for example an enzyme which catalyzes modifications to nucleic acids, including, but not limited to, catalytic oligonucleotides, nucleases (e.g., programmable nucleases), polymerases, kinases, phosphatases, or the like. Upon recognition of a target nucleic acid by the programmable nuclease, the activated programmable nuclease's transcleavage activity may be leveraged to activate a signal amplification cascade by activating one or more signal amplifiers. The signal amplifiers may be capable of cleaving a reporter and generating a signal therefrom. In some instances, the signal amplifier may catalyze reactions which may be independent of reporter cleavage, for example an HRP-mediated redox reaction. The signal amplification cascade may include a positive feedback loop such that activation of the signal amplifier results in exponential signal amplification compared to the programmable nuclease-generated signal alone.

Provided herein are methods and compositions for performing a nucleic acid detection assay. In some embodiments, provided herein is a composition comprising a catalytic oligonucleotide, a programmable nuclease, a guide nucleic acid, and a reporter molecule. The composition can further comprise a target nucleic acid. The target nucleic acid can be in a sample. In some examples, the guide nucleic acid is configured to hybridize to a segment of a target nucleic acid. In some embodiments, the catalytic oligonucleotide is circularized. In some embodiments, the catalytic oligonucleotide is configured to become activated upon cleavage by the programmable nuclease to form a secondary structure capable of cleaving a reporter molecule. In some embodiments, the catalytic oligonucleotide is bound to a blocker oligonucleotide. In some embodiments, the catalytic oligonucleotide is configured to become activated upon cleavage of the blocker oligonucleotide by the programmable nuclease to form a secondary structure capable of cleaving a reporter molecule. Often, the target nucleic acid detected is at a low concentration in the sample.

In some embodiments, provided herein is a composition comprising a first catalytic oligonucleotide, a second catalytic oligonucleotide, a first blocker oligonucleotide, a second blocker oligonucleotide, a programmable nuclease, a guide nucleic acid, and a reporter molecule. In some embodiments, the first catalytic oligonucleotide is bound to the first blocker oligonucleotide and the second catalytic oligonucleotide is bound to the second blocker oligonucleotide. The composition can further comprise a target nucleic acid. The target nucleic acid can be in a sample. In some examples, the guide nucleic acid is configured to hybridize to a segment of a target nucleic acid. In some embodiments, the first catalytic oligonucleotide is configured to become activated upon cleavage of the first blocker oligonucleotide by the programmable nuclease so that the first catalytic oligonucleotide forms a secondary structure capable of cleaving a reporter molecule and capable of cleaving the second blocker oligonucleotide. In some embodiments, the second catalytic oligonucleotide is configured to become activated upon cleavage of the second blocker oligonucleotide by the first catalytic oligonucleotide to form a secondary structure capable of cleaving the first blocker oligonucleotide. Often, the target nucleic acid is at a low concentration in the sample.

Disclosed herein are non-naturally occurring compositions and systems comprising an effector protein (e.g., a programmable nuclease) and an engineered guide nucleic acid, which may simply be referred to herein as a guide nucleic acid. In general, an engineered effector protein and an engineered guide nucleic acid refer to an effector protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some instances, the guide nucleic acid comprises a non-natural nucleobase sequence. In some instances, the non-natural sequence is a nucleobase sequence that is not found in nature. The non-natural sequence may comprise a portion of a naturally-occurring sequence, wherein the portion of the naturally-occurring sequence is not present in nature, absent the remainder of the naturally-occurring sequence. In some instances, the guide nucleic acid comprises two naturally-occurring sequences arranged in an order or proximity that is not observed in nature. In some instances, compositions and systems comprise a ribonucleotide complex comprising an effector protein and a guide nucleic acid that do not occur together in nature. Engineered guide nucleic acids may comprise a first sequence and a second sequence that do not occur naturally together. For example, an engineered guide nucleic acid may comprise a sequence of a naturally-occurring repeat region and a spacer region that is complementary to a naturally-occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence located at a 3′ or 5′ end of the guide nucleic acid, or between the first and second sequences of the guide nucleic acid. For example, an engineered guide nucleic acid may comprise a naturally occurring CRISPR RNA (crRNA) and trans-activating crRNA (tracrRNA) coupled by a linker sequence.

In some instances, compositions and systems described herein comprise an engineered effector protein that is similar to a naturally occurring effector protein. The engineered effector protein may lack a portion of the naturally occurring effector protein. The effector protein may comprise a mutation relative to the naturally-occurring effector protein, wherein the mutation is not found in nature. The effector protein may also comprise at least one additional amino acid relative to the naturally-occurring effector protein. For example, the effector protein may comprise an addition of a nuclear localization signal relative to the natural occurring effector protein. In certain embodiments, the nucleotide sequence encoding the effector protein is codon optimized (e.g., for expression in a eukaryotic cell) relative to the naturally occurring sequence.

### Compositions for Detection of a Target Nucleic Acid

Target nucleic acids can be detected using compositions as described herein. Compositions as described herein can comprise programmable nucleases, guide nucleic acids, signal amplifiers (e.g., catalytic oligonucleotides), blocker oligonucleotides, reporter molecules, target nucleic acids, and/or buffers. In some embodiments, a target nucleic acid is directly detected without target nucleic acid amplification. Direct detection of target nucleic acids can eliminate or decrease the need for intermediate steps, for example reverse transcription or nucleic acid amplification, required by existing programmable nuclease-based sequence detection methods. Elimination of the intermediate steps can decrease time to assay result and reduce labor and reagent costs.

### Programmable Nucleases

Disclosed herein are programmable nucleases and uses thereof, e.g., detection and editing of target nucleic acids. In some cases, a programmable nuclease is capable of being activated when complexed with the guide nucleic acid and the target nucleic acid segment. A programmable nuclease can be capable of being activated when complexed with a guide nucleic acid and the target sequence. The programmable nuclease can be activated upon binding of the guide nucleic acid to its target nucleic acid and can non-specifically degrade a non-target nucleic acid in its environment. The programmable nuclease has trans cleavage activity once activated. A programmable nuclease can be a Cas protein (also referred to, interchangeably, as a Cas nuclease or Cas effector protein). A guide nucleic acid (e.g., crRNA) and Cas protein can form a CRISPR enzyme.

The compositions as disclosed herein can comprise a programmable nuclease for the detection of a target nucleic acid. The programmable nuclease can be activated upon binding of a guide nucleic acid to its target nucleic acid to non-specifically cleave nearby nucleic acids. This non-specific cleavage can be referred to as trans cleavage or trans collateral cleavage. The guide nucleic acid can be a guide nucleic acid as described herein. In the compositions and methods as described herein, the trans collateral cleavage activity of a programmable nuclease can cleave nearby reporter molecules, catalytic oligonucleotides (e.g., circular catalytic oligonucleotides), blocker oligonucleotides, or any combination thereof.

The systems and methods of the present disclosure can be implemented using a device that is compatible with a plurality of programmable nucleases. The device can comprise a plurality of programmable nuclease probes comprising the plurality of programmable nucleases and one or more corresponding guide nucleic acids. The plurality of programmable nuclease probes can be the same. Alternatively, the plurality of programmable nuclease probes can be different. For example, the plurality of programmable nuclease probes can comprise different programmable nucleases and/or different guide nucleic acids associated with the programmable nucleases.

As used herein, a programmable nuclease generally refers to any enzyme that can cleave nucleic acid. The programmable nuclease can be any enzyme that can be or has been designed, modified, or engineered by human contribution so that the enzyme targets or cleaves the nucleic acid in a sequence-specific manner. Programmable nucleases can include, for example, zinc-finger nucleases (ZFNs), transcription activator-like effector nucleases (TALENs), and/or RNA-guided nucleases such as the bacterial clustered regularly interspaced short palindromic repeat (CRISPR)-Cas (CRISPR-associated) nucleases or Cpfl. Programmable nucleases can also include, for example, PfAgo and/or NgAgo.

ZFNs can cut genetic material in a sequence-specific matter and can be designed, or programmed, to target specific viral targets. A ZFN is composed of two domains: a DNA-binding zinc-finger protein linked to the Fokl nuclease domain. The DNA-binding zinc-finger protein is fused with the non-specific Fokl cleave domain to create ZFNs. The protein will typically dimerize for activity. Two ZFN monomers form an active nuclease; each monomer binds to adjacent half-sites on the target. The sequence specificity of ZFNs is determined by ZFPs. Each zinc-finger recognizes a 3-bp DNA sequence, and 3-6 zinc-fingers are used to generate a single ZFN subunit that binds to DNA sequences of 9-18 bp. The DNA-binding specificities of zinc-fingers is altered by mutagenesis. New ZFPs are programmed by modular assembly of pre-characterized zinc fingers.

Transcription activator-like effector nucleases (TALENs) can cut genetic material in a sequence-specific matter and can be designed, or programmed, to target specific viral targets. TALENs contain the Fokl nuclease domain at their carboxyl termini and a class of DNA binding domains known as transcription activator-like effectors (TALEs). TALENs are composed of tandem arrays of 33-35 amino acid repeats, each of which recognizes a single base-pair in the major groove of target viral DNA. The nucleotide specificity of a domain comes from the two amino acids at positions 12 and 13 where Asn-Asn, Asn-Ile, His-Asp and Asn-Gly recognize guanine, adenine, cytosine and thymine, respectively. That pattern allows one to program TALENs to target various nucleic acids.

Several programmable nucleases are consistent with the compositions and methods of the present disclosure. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid. The CRISPR-Cas nucleoprotein complex can comprise a Cas protein (also referred to as a Cas nuclease) complexed with a guide nucleic acid, which can also be referred to as CRISPR enzyme. A programmable nuclease can be a Cas protein (also referred to, interchangeably, as a Cas nuclease). A guide nucleic acid can be a CRISPR RNA (crRNA). Sometimes, a guide nucleic acid comprises a crRNA and a trans-activating crRNA (tracrRNA). The CRISPR/Cas system used to detect a modified target nucleic acids can comprise CRISPR RNAs (crRNAs), trans-activating crRNAs (tracrRNAs), Cas proteins, and reporter molecules.

A crRNA and Cas protein can form a CRISPR enzyme. For example, CRISPR/Cas enzymes are programmable nucleases used in the compositions and methods as disclosed herein. CRISPR/Cas enzymes can include any of the known Classes and Types of CRISPR/Cas enzymes. Programmable nucleases disclosed herein include Class 1 CRISPR/Cas enzymes, such as the Type I, Type IV, or Type III CRISPR/Cas enzymes. Programmable nucleases disclosed herein also include the Class 2 CRISPR/Cas enzymes, such as the Type II, Type V, and Type VI CRISPR/Cas enzymes. Preferable programmable nucleases included in the compositions as disclosed herein and methods of use thereof include a Type V or Type VI CRISPR/Cas enzyme. In some cases, the programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA. The catalytic oligonucleotide cleaved by the trans cleavage activity of a programmable nuclease can comprise RNA, DNA, or both. The blocker oligonucleotide cleaved by the trans cleavage activity of a programmable nuclease can comprise RNA, DNA, or both.

In some instances, the programmable nuclease is a Type VI Cas protein. In some embodiments, the Type VI CRISPR/Cas enzyme is a Cas13 nuclease. The general architecture of a Cas13 protein includes an N-terminal domain and two HEPN (higher eukaryotes and prokaryotes nucleotide-binding) domains separated by two helical domains. The HEPN domains each comprise aR-X₄-H motif. Shared features across Cas13 proteins include that upon binding of the crRNA of the engineered guide nucleic acid to a target nucleic acid, the protein undergoes a conformational change to bring together the HEPN domains and form a catalytically active RNase. Thus, two activatable HEPN domains are characteristic of a Cas13 nuclease of the present disclosure. However, programmable Cas13 nucleases also consistent with the present disclosure include Cas13 nucleases comprising mutations in the HEPN domain that enhance the Cas13 proteins cleavage efficiency or mutations that catalytically inactivate the HEPN domains. Programmable Cas13 nucleases consistent with the present disclosure also Cas13 nucleases comprising catalytic components. In some instances, the Cas effector is a Cas 13 effector. In some instances, the Cas13 effector is a Cas13a, a Cas13b, a Cas 13c, a Cas 13d, or a Cas 13e effector protein.

The programmable nuclease can be Cas13. Sometimes the Cas13 can be Cas13a, Cas13b, Cas13c, Cas13d, or Cas13e. In some cases, the programmable nuclease can be Mad7 or Mad2.

A Cas13 nuclease can be a Cas13a protein (also referred to as “c2c2”), a Cas13b protein, a Cas13c protein, a Cas13d protein, or a Cas13e protein. Example C2c2 proteins are set forth as SEQ ID NO: 18-SEQ ID NO: 35. In some cases, a subject C2c2 protein includes an amino acid sequence having 80% or more (e.g., 85% or more, 90% or more, 95% or more, 98% or more, 99% or more, 99.5% or more, or 100%) amino acid sequence identity with the amino acid sequence set forth in any one of SEQ ID NO: 18-SEQ ID NO: 35. In some cases, a suitable C2c2 polypeptide comprises an amino acid sequence having at least 80%, at least 85%, at least 90%, at least 95%, at least 98%, at least 99%, or 100%, amino acid sequence identity to the Listeria seeligeri C2c2 amino acid sequence set forth in SEQ ID NO: 18. In some cases, a suitable C2c2 polypeptide comprises an amino acid sequence having at least 80%, at least 85%, at least 90%, at least 95%, at least 98%, at least 99%, or 100%, amino acid sequence identity to the Leptotrichia buccalis C2c2 amino acid sequence set forth in SEQ ID NO: 19. In some cases, a suitable C2c2 polypeptide comprises an amino acid sequence having at least 80%, at least 85%, at least 90%, at least 95%, at least 98%, at least 99%, or 100%, amino acid sequence identity to the Rhodobacter capsulatus C2c2 amino acid sequence set forth in SEQ ID NO: 21. In some cases, a suitable C2c2 polypeptide comprises an amino acid sequence having at least 80%, at least 85%, at least 90%, at least 95%, at least 98%, at least 99%, or 100%, amino acid sequence identity to the Carnobacterium gallinarum C2c2 amino acid sequence set forth in SEQ ID NO: 22. In some cases, a suitable C2c2 polypeptide comprises an amino acid sequence having at least 80%, at least 85%, at least 90%, at least 95%, at least 98%, at least 99%, or 100%, amino acid sequence identity to the Herbinix hemicellulosilytica C2c2 amino acid sequence set forth in SEQ ID NO: 23. In some cases, the C2c2 protein includes an amino acid sequence having 80% or more amino acid sequence identity with the Leptotrichia buccalis (Lbu) C2c2 amino acid sequence set forth in SEQ ID NO: 19. In some cases, the C2c2 protein is a Leptotrichia buccalis (Lbu) C2c2 protein (e.g., see SEQ ID NO: 19). In some cases, the C2c2 protein includes the amino acid sequence set forth in any one of SEQ ID NO: 18-SEQ ID NO: 35. In some cases, a C2c2 protein used in a method of the present disclosure is not a C2c2 polypeptide having at least 80%, at least 85%, at least 90%, at least 95%, at least 98%, at least 99%, or 100%, amino acid sequence identity to the Lsh C2c2 polypeptide set forth in SEQ ID NO: 20. Exemplary Cas13 protein sequences are set forth in SEQ ID NO: 18-SEQ ID NO: 35. TABLE 1, below, shows exemplary Cas13 programmable nuclease sequences of the present disclosure.

TABLE 1
Exemplary Cas13 Protein Sequences

| SEQ | | |
|---|---|---|
| ID | | |
| NO | Description | Sequence |
| SEQ | Listeria | MWISIKTLIHHLGVLFFCDYMYNRR |
| ID | seeligeri C2c2 | EKKIIEVKTMRITKVEVDRKKVLIS |
| NO: | amino acid | RDKNGGKLVYENEMQDNTEQIMHHK |
| 18 | sequence | KSSFYKSVVNKTICRPEQKQMKKLV |
| | | HGLLQENSQEKIKVSDVTKLNISNF |
| | | LNHRFKKSLYYFPENSPDKSEEYRI |
| | | EINLSQLLEDSLKKQQGTFICWESF |
| | | SKDMELYINWAENYISSKTKLIKKS |
| | | IRNNRIQSTESRSGQLMDRYMKDIL |
| | | NKNKPFDIQSVSEKYQLEKLTSALK |
| | | ATFKEAKKNDKEINYKLKSTLQNHE |
| | | RQIIEELKENSELNQFNIEIRKHLE |
| | | TYFPIKKTNRKVGDIRNLEIGEIQK |
| | | IVNHRLKNKIVQRILQEGKLASYEI |
| | | ESTVNSNSLQKIKIEEAFALKFINA |
| | | CLFASNNLRNMVYPVCKKDILMIGE |
| | | FKNSFKEIKHKKFIRQWSQFFSQEI |
| | | TVDDIELASWGLRGAIAPIRNEIIH |
| | | LKKHSWKKFFNNPTFKVKKSKIING |
| | | KTKDVTSEFLYKETLFKDYFYSELD |
| | | SVPELIINKMESSKILDYYSSDQLN |
| | | QVFTIPNFELSLLTSAVPFAPSFKR |
| | | VYLKGFDYQNQDEAQPDYNLKLNIY |
| | | NEKAFNSEAFQAQYSLFKMVYYQVF |
| | | LPQFTTNNDLFKSSVDFILTLNKER |
| | | KGYAKAFQDIRKMNKDEKPSEYMSY |
| | | IQSQLMLYQKKQEEKEKINHFEKFI |
| | | NQVFIKGFNSFIEKNRLTYICHPTK |
| | | NTVPENDNIEIPFHTDMDDSNIAFW |
| | | LMCKLLDAKQLSELRNEMIKFSCSL |
| | | QSTEEISTFTKAREVIGLALLNGEK |
| | | GCNDWKELFDDKEAWKKNMSLYVSE |
| | | ELLQSLPYTQEDGQTPVINRSIDLV |
| | | KKYGTETILEKLFSSSDDYKVSAKD |
| | | IAKLHEYDVTEKIAQQESLHKQWIE |
| | | KPGLARDSAWTKKYQNVINDISNYQ |
| | | WAKTKVELTQVRHLHQLTIDLLSRL |
| | | AGYMSIADRDFQFSSNYILERENSE |
| | | YRVTSWILLSENKNKNKYNDYELYN |
| | | LKNASIKVSSKNDPQLKVDLKQLRL |
| | | TLEYLELFDNRLKEKRNNISHFNYL |
| | | NGQLGNSILELFDDARDVLSYDRKL |
| | | KNAVSKSLKEILSSHGMEVTFKPLY |
| | | QTNHHLKIDKLQPKKIHHLGEKSTV |
| | | SSNQVSNEYCQLVRTLLTMK |
| SEQ | Leptotrichia | MKVTKVGGISHKKYTSEGRLVKSES |
| ID | buccalis (Lbu) | EENRTDERLSALLNMRLDMYIKNPS |
| NO: | C2c2 amino | STETKENQKRIGKLKKFFSNKMVYL |
| 19 | acid sequence | KDNTLSLKNGKKENIDREYSETDIL |
| | | ESDVRDKKNFAVLKKIYLNENVNSE |
| | | ELEVFRNDIKKKLNKINSLKYSFEK |
| | | NKANYQKINENNIEKVEGKSKRNII |
| | | YDYYRESAKRDAYVSNVKEAFDKLY |
| | | KEEDIAKLVLEIENLTKLEKYKIRE |
| | | FYHEIIGRKNDKENFAKIIYEEIQN |
| | | VNNMKELIEKVPDMSELKKSQVFYK |
| | | YYLDKEELNDKNIKYAFCHFVEIEM |
| | | SQLLKNYVYKRLSNISNDKIKRIFE |
| | | YQNLKKLIENKLLNKLDTYVRNCGK |
| | | YNYYLQDGEIATSDFIARNRQNEAF |
| | | LRNIIGVSSVAYFSLRNILETENEN |
| | | DITGRMRGKTVKNNKGEEKYVSGEV |
| | | DKIYNENKKNEVKENLKMFYSYDFN |
| | | MDNKNEIEDFFANIDEAISSIRHGI |
| | | VHFNLELEGKDIFAFKNIAPSEISK |
| | | KMFQNEINEKKLKLKIFRQLNSANV |
| | | FRYLEKYKILNYLKRTRFEFVNKNI |
| | | PFVPSFTKLYSRIDDLKNSLGIYWK |
| | | TPKTNDDNKTKEIIDAQIYLLKNIY |
| | | YGEFLNYFMSNNGNFFEISKEIIEL |
| | | NKNDKRNLKTGFYKLQKFEDIQEKI |
| | | PKEYLANIQSLYMINAGNQDEEEKD |
| | | TYIDFIQKIFLKGFMTYLANNGRLS |
| | | LIYIGSDEETNTSLAEKKQEFDKFL |
| | | KKYEQNNNIKIPYEINEFLREIKLG |
| | | NILKYTERLNMFYLILKLLNHKELT |
| | | NLKGSLEKYQSANKEEAFSDQLELI |
| | | NLLNLDNNRVTEDFELEADEIGKFL |
| | | DFNGNKVKDNKELKKFDTNKIYFDG |
| | | ENIIKHRAFYNIKKYGMLNLLEKIA |
| | | DKAGYKISIEELKKYSNKKNEIEKN |
| | | HKMQENLHRKYARPRKDEKFTDEDY |
| | | ESYKQAIENIEEYTHLKNKVEFNEL |
| | | NLLQGLLLRILHRLVGYTSIWERDL |
| | | RFRLKGEFPENQYIEEIFNFENKKN |
| | | VKYKGGQIVEKYIKFYKELHQNDEV |
| | | KINKYSSANIKVLKQEKKDLYIRNY |
| | | IAHFNYIPHAEISLLEVLENLRKLL |
| | | SYDRKLKNAVMKSVVDILKEYGFVA |
| | | TFKIGADKKIGIQTLESEKIVHLKN |
| | | LKKKKLMTDRNSEELCKLVKIMFEY |
| | | KMEEKKSEN |
| SEQ | Leptotrichia | MGNLFGHKRWYEVRDKKDFKIKRKV |
| ID | shahii (Lsh) | KVKRNYDGNKYILNINENNNKEKID |
| NO: | C2c2 protein | NNKFIRKYINYKKNDNILKEFTRKF |
| 20 | | HAGNILFKLKGKEGIIRIENNDDFL |
| | | ETEEVVLYIEAYGKSEKLKALGITK |
| | | KKIIDEAIRQGITKDDKKIEIKRQE |
| | | NEEEIEIDIRDEYTNKTLNDCSIIL |
| | | RIIENDELETKKSIYEIFKNINMSL |
| | | YKIIEKIIENETEKVFENRYYEEHL |
| | | REKLLKDDKIDVILTNFMEIREKIK |
| | | SNLEILGFVKFYLNVGGDKKKSKNK |
| | | KMLVEKILNINVDLTVEDIADFVIK |
| | | ELEFWNITKRIEKVKKVNNEFLEKR |
| | | RNRTYIKSYVLLDKHEKFKIERENK |
| | | KDKIVKFFVENIKNNSIKEKIEKIL |
| | | AEFKIDELIKKLEKELKKGNCDTEI |
| | | FGIFKKHYKVNFDSKKFSKKSDEEK |
| | | ELYKIIYRYLKGRIEKILVNEQKVR |
| | | LKKMEKIEIEKILNESILSEKILKR |
| | | VKQYTLEHIMYLGKLRHNDIDMTTV |
| | | NTDDFSRLHAKEELDLELITFFAST |
| | | NMELNKIFSRENINNDENIDFFGGD |
| | | REKNYVLDKKILNSKIKIIRDLDFI |
| | | DNKNNITNNFIRKFTKIGTNERNRI |
| | | LHAISKERDLQGTQDDYNKVINIIQ |
| | | NLKISDEEVSKALNLDVVFKDKKNI |
| | | ITKINDIKISEENNNDIKYLPSFSK |
| | | VLPEILNLYRNNPKNEPFDTIETEK |
| | | IVLNALIYVNKELYKKLILEDDLEE |
| | | NESKNIFLQELKKTLGNIDEIDENI |
| | | IENYYKNAQISASKGNNKAIKKYQK |
| | | KVIECYIGYLRKNYEELFDFSDFKM |
| | | NIQEIKKQIKDINDNKTYERITVKT |
| | | SDKTIVINDDFEYIISIFALLNSNA |
| | | VINKIRNRFFATSVWLNTSEYQNII |
| | | DILDEIMQLNTLRNECITENWNLNL |
| | | EEFIQKMKEIEKDFDDFKIQTKKEI |
| | | FNNYYEDIKNNILTEFKDDINGCDV |
| | | LEKKLEKIVIFDDETKFEIDKKSNI |
| | | LQDEQRKLSNINKKDLKKKVDQYIK |
| | | DKDQEIKSKILCRIIFNSDFLKKYK |
| | | KEIDNLIEDMESENENKFQEIYYPK |
| | | ERKNELYIYKKNLFLNIGNPNFDKI |
| | | YGLISNDIKMADAKFLFNIDGKNIR |
| | | KNKISEIDAILKNLNDKLNGYSKEY |
| | | KEKYIKKLKENDDFFAKNIQNKNYK |
| | | SFEKDYNRVSEYKKIRDLVEFNYLN |
| | | KIESYLIDINWKLAIQMARFERDMH |
| | | YIVNGLRELGIIKLSGYNTGISRAY |
| | | PKRNGSDGFYTTTAYYKFFDEESYK |
| | | KFEKICYGFGIDLSENSEINKPENE |
| | | SIRNYISHFYIVRNPFADYSIAEQI |
| | | DRVSNLLSYSTRYNNSTYASVFEVF |
| | | KKDVNLDYDELKKKFKLIGNNDILE |
| | | RLMKPKKVSVLELESYNSDYIKNLI |
| | | IELLTKIENTNDTL |
| SEQ | Rhodobacter | MQIGKVQGRTISEFGDPAGGLKRKI |
| ID | capsulatus | STDGKNRKELPAHLSSDPKALIGQW |
| NO: | C2c2 amino | ISGIDKIYRKPDSRKSDGKAIHSPT |
| 21 | acid sequence | PSKMQFDARDDLGEAFWKLVSEAGL |
| | | AQDSDYDQFKRRLHPYGDKFQPADS |
| | | GAKLKFEADPPEPQAFHGRWYGAMS |
| | | KRGNDAKELAAALYEHLHVDEKRID |
| | | GQPKRNPKTDKFAPGLVVARALGIE |
| | | SSVLPRGMARLARNWGEEEIQTYFV |
| | | VDVAASVKEVAKAAVSAAQAFDPPR |
| | | QVSGRSLSPKVGFALAEHLERVTGS |
| | | KRCSFDPAAGPSVLALHDEVKKTYK |
| | | RLCARGKNAARAFPADKTELLALMR |
| | | HTHENRVRNQMVRMGRVSEYRGQQA |
| | | GDLAQSHYWTSAGQTEIKESEIFVR |
| | | LWVGAFALAGRSMKAWIDPMGKIVN |
| | | TEKNDRDLTAAVNIRQVISNKEMVA |
| | | EAMARRGIYFGETPELDRLGAEGNE |
| | | GFVFALLRYLRGCRNQTFHLGARAG |
| | | FLKEIRKELEKTRWGKAKEAEHVVL |
| | | TDKTVAAIRAIIDNDAKALGARLLA |
| | | DLSGAFVAHYASKEHFSTLYSEIVK |
| | | AVKDAPEVSSGLPRLKLLLKRADGV |
| | | RGYVHGLRDTRKHAFATKLPPPPAP |
| | | RELDDPATKARYIALLRLYDGPFRA |
| | | YASGITGTALAGPAARAKEAATALA |
| | | QSVNVTKAYSDVMEGRSSRLRPPND |
| | | GETLREYLSALTGETATEFRVQIGY |
| | | ESDSENARKQAEFIENYRRDMLAFM |
| | | FEDYIRAKGFDWILKIEPGATAMTR |
| | | APVLPEPIDTRGQYEHWQAALYLVM |
| | | HFVPASDVSNLLHQLRKWEALQGKY |
| | | ELVQDGDATDQADARREALDLVKRF |
| | | RDVLVLFLKTGEARFEGRAAPFDLK |
| | | PFRALFANPATFDRLFMATPTTARP |
| | | AEDDPEGDGASEPELRVARTLRGLR |
| | | QIARYNHMAVLSDLFAKHKVRDEEV |
| | | ARLAEIEDETQEKSQIVAAQELRTD |
| | | LHDKVMKCHPKTISPEERQSYAAAI |
| | | KTIEEHRFLVGRVYLGDHLRLHRLM |
| | | MDVIGRLIDYAGAYERDTGTFLINA |
| | | SKQLGAGADWAVTIAGAANTDARTQ |
| | | TRKDLAHFNVLDRADGTPDLTALVN |
| | | RAREMMAYDRKRKNAVPRSILDMLA |
| | | RLGLTLKWQMKDHLLQDATITQAAI |
| | | KHLDKVRLTVGGPAAVTEARFSQDY |
| | | LQMVAAVFNGSVQNPKPRRRDDGDA |
| | | WHKPPKPATAQSQPDQKPPNKAPSA |
| | | GSRLPPPQVGEVYEGVVVKVIDTGS |
| | | LGFLAVEGVAGNIGLHISRLRRIRE |
| | | DAIIVGRRYRFRVEIYVPPKSNTSK |
| | | LNAADLVRID |
| SEQ | Carnobacterium | MRITKVKIKLDNKLYQVTMQKEEKY |
| ID | gallinarum | GTLKLNEESRKSTAEILRLKKASFN |
| NO: | C2c2 amino | KSFHSKTINSQKENKNATIKKNGDY |
| 22 | acid sequence | ISQIFEKLVGVDTNKNIRKPKMSLT |
| | | DLKDLPKKDLALFIKRKFKNDDIVE |
| | | IKNLDLISLFYNALQKVPGEHFTDE |
| | | SWADFCQEMMPYREYKNKFIERKII |
| | | LLANSIEQNKGFSINPETFSKRKRV |
| | | LHQWAIEVQERGDFSILDEKLSKLA |
| | | EIYNFKKMCKRVQDELNDLEKSMKK |
| | | GKNPEKEKEAYKKQKNFKIKTIWKD |
| | | YPYKTHIGLIEKIKENEELNQFNIE |
| | | IGKYFEHYFPIKKERCTEDEPYYLN |
| | | SETIATTVNYQLKNALISYLMQIGK |
| | | YKQFGLENQVLDSKKLQEIGIYEGF |
| | | QTKFMDACVFATSSLKNIIEPMRSG |
| | | DILGKREFKEAIATSSFVNYHHFFP |
| | | YFPFELKGMKDRESELIPFGEQTEA |
| | | KQMQNIWALRGSVQQIRNEIFHSFD |
| | | KNQKFNLPQLDKSNFEFDASENSTG |
| | | KSQSYIETDYKFLFEAEKNQLEQFF |
| | | IERIKSSGALEYYPLKSLEKLFAKK |
| | | EMKFSLGSQVVAFAPSYKKLVKKGH |
| | | SYQTATEGTANYLGLSYYNRYELKE |
| | | ESFQAQYYLLKLIYQYVFLPNFSQG |
| | | NSPAFRETVKAILRINKDEARKKMK |
| | | KNKKFLRKYAFEQVREMEFKETPDQ |
| | | YMSYLQSEMREEKVRKAEKNDKGFE |
| | | KNITMNFEKLLMQIFVKGFDVFLTT |
| | | FAGKELLLSSEEKVIKETEISLSKK |
| | | INEREKTLKASIQVEHQLVATNSAI |
| | | SYWLFCKLLDSRHLNELRNEMIKFK |
| | | QSRIKFNHTQHAELIQNLLPIVELT |
| | | ILSNDYDEKNDSQNVDVSAYFEDKS |
| | | LYETAPYVQTDDRTRVSFRPILKLE |
| | | KYHTKSLIEALLKDNPQFRVAATDI |
| | | QEWMHKREEIGELVEKRKNLHTEWA |
| | | EGQQTLGAEKREEYRDYCKKIDRFN |
| | | WKANKVTLTYLSQLHYLITDLLGRM |
| | | VGFSALFERDLVYFSRSFSELGGET |
| | | YHISDYKNLSGVLRLNAEVKPIKIK |
| | | NIKVIDNEENPYKGNEPEVKPFLDR |
| | | LHAYLENVIGIKAVHGKIRNQTAHL |
| | | SVLQLELSMIESMNNLRDLMAYDRK |
| | | LKNAVTKSMIKILDKHGMILKLKID |
| | | ENHKNFEIESLIPKEIIHLKDKAIK |
| | | TNQVSEEYCQLVLALLTTNPGNQLN |
| SEQ | Herbinix | MKLTRRRISGNSVDQKITAAFYRDM |
| ID | hemi- | SQGLLYYDSEDNDCTDKVIESMDFE |
| NO: | cellulosilytica | RSWRGRILKNGEDDKNPFYMFVKGL |
| 23 | C2c2 | VGSNDKIVCEPIDVDSDPDNLDILI |
| | amino acid | NKNLTGFGRNLKAPDSNDTLENLIR |
| | sequence | KIQAGIPEEEVLPELKKIKEMIQKD |
| | | IVNRKEQLLKSIKNNRIPFSLEGSK |
| | | LVPSTKKMKWLFKLIDVPNKTFNEK |
| | | MLEKYWEIYDYDKLKANITNRLDKT |
| | | DKKARSISRAVSEELREYHKNLRTN |
| | | YNRFVSGDRPAAGLDNGGSAKYNPD |
| | | KEEFLLFLKEVEQYFKKYFPVKSKH |
| | | SNKSKDKSLVDKYKNYCSYKVVKKE |
| | | VNRSIINQLVAGLIQQGKLLYYFYY |
| | | NDTWQEDFLNSYGLSYIQVEEAFKK |
| | | SVMTSLSWGINRLTSFFIDDSNTVK |
| | | FDDITTKKAKEAIESNYFNKLRTCS |
| | | RMQDHFKEKLAFFYPVYVKDKKDRP |
| | | DDDIENLIVLVKNAIESVSYLRNRT |
| | | FHFKESSLLELLKELDDKNSGQNKI |
| | | DYSVAAEFIKRDIENLYDVFREQIR |
| | | SLGIAEYYKADMISDCFKTCGLEFA |
| | | LYSPKNSLMPAFKNVYKRGANLNKA |
| | | YIRDKGPKETGDQGQNSYKALEEYR |
| | | ELTWYIEVKNNDQSYNAYKNLLQLI |
| | | YYHAFLPEVRENEALITDFINRTKE |
| | | WNRKETEERLNTKNNKKHKNFDEND |
| | | DITVNTYRYESIPDYQGESLDDYLK |
| | | VLQRKQMARAKEVNEKEEGNNNYIQ |
| | | FIRDVVVWAFGAYLENKLKNYKNEL |
| | | QPPLSKENIGLNDTLKELFPEEKVK |
| | | SPFNIKCRFSISTFIDNKGKSTDNT |
| | | SAEAVKTDGKEDEKDKKNIKRKDLL |
| | | CFYLFLRLLDENEICKLQHQFIKYR |
| | | CSLKERRFPGNRTKLEKETELLAEL |
| | | EELMELVRFTMPSIPEISAKAESGY |
| | | DTMIKKYFKDFIEKKVFKNPKTSNL |
| | | YYHSDSKTPVTRKYMALLMRSAPLH |
| | | LYKDIFKGYYLITKKECLEYIKLSN |
| | | IIKDYQNSLNELHEQLERIKLKSEK |
| | | QNGKDSLYLDKKDFYKVKEYVENLE |
| | | QVARYKHLQHKINFESLYRIFRIHV |
| | | DIAARMVGYTQDWERDMHFLFKALV |
| | | YNGVLEERRFEAIFNNNDDNNDGRI |
| | | VKKIQNNLNNKNRELVSMLCWNKKL |
| | | NKNEFGAIIWKRNPIAHLNHFTQTE |
| | | QNSKSSLESLINSLRILLAYDRKRQ |
| | | NAVTKTINDLLLNDYHIRIKWEGRV |
| | | DEGQIYFNIKEKEDIENEPIIHLKH |
| | | LHKKDCYIYKNSYMFDKQKEWICNG |
| | | IKEEVYDKSILKCIGNLFKFDYEDK |
| | | NKSSANPKHT |
| SEQ | Paludibacter | MRVSKVKVKDGGKDKMVLVHRKTTG |
| ID | propionicigenes | AQLVYSGQPVSNETSNILPEKKRQS |
| NO: | C2c2 | FDLSTLNKTIIKFDTAKKQKLNVDQ |
| 24 | amino acid | YKIVEKIFKYPKQELPKQIKAEEIL |
| | sequence | PFLNHKFQEPVKYWKNGKEESFNLT |
| | | LLIVEAVQAQDKRKLQPYYDWKTWY |
| | | IQTKSDLLKKSIENNRIDLTENLSK |
| | | RKKALLAWETEFTASGSIDLTHYHK |
| | | VYMTDVLCKMLQDVKPLTDDKGKIN |
| | | TNAYHRGLKKALQNHQPAIFGTREV |
| | | PNEANRADNQLSIYHLEVVKYLEHY |
| | | FPIKTSKRRNTADDIAHYLKAQTLK |
| | | TTIEKQLVNAIRANIIQQGKTNHHE |
| | | LKADTTSNDLIRIKTNEAFVLNLTG |
| | | TCAFAANNIRNMVDNEQTNDILGKG |
| | | DFIKSLLKDNTNSQLYSFFFGEGLS |
| | | TNKAEKETQLWGIRGAVQQIRNNVN |
| | | HYKKDALKTVFNISNFENPTITDPK |
| | | QQTNYADTIYKARFINELEKIPEAF |
| | | AQQLKTGGAVSYYTIENLKSLLTTF |
| | | QFSLCRSTIPFAPGFKKVFNGGINY |
| | | QNAKQDESFYELMLEQYLRKENFAE |
| | | ESYNARYFMLKLIYNNLFLPGFTTD |
| | | RKAFADSVGFVQMQNKKQAEKVNPR |
| | | KKEAYAFEAVRPMTAADSIADYMAY |
| | | VQSELMQEQNKKEEKVAEETRINFE |
| | | KFVLQVFIKGFDSFLRAKEFDFVQM |
| | | PQPQLTATASNQQKADKLNQLEASI |
| | | TADCKLTPQYAKADDATHIAFYVFC |
| | | KLLDAAHLSNLRNELIKFRESVNEF |
| | | KFHHLLEIIEICLLSADVVPTDYRD |
| | | LYSSEADCLARLRPFIEQGADITNW |
| | | SDLFVQSDKHSPVIHANIELSVKYG |
| | | TTKLLEQIINKDTQFKTTEANFTAW |
| | | NTAQKSIEQLIKQREDHHEQWVKAK |
| | | NADDKEKQERKREKSNFAQKFIEKH |
| | | GDDYLDICDYINTYNWLDNKMHFVH |
| | | LNRLHGLTIELLGRMAGFVALFDRD |
| | | FQFFDEQQIADEFKLHGFVNLHSID |
| | | KKLNEVPTKKIKEIYDIRNKIIQIN |
| | | GNKINESVRANLIQFISSKRNYYNN |
| | | AFLHVSNDEIKEKQMYDIRNHIAHF |
| | | NYLTKDAADFSLIDLINELRELLHY |
| | | DRKLKNAVSKAFIDLFDKHGMILKL |
| | | KLNADHKLKVESLEPKKIYHLGSSA |
| | | KDKPEYQYCTNQVMMAYCNMCRSLL |
| | | EMKK |
| SEQ | Leptotrichia | MYMKITKIDGVSHYKKQDKGILKKK |
| ID | wadei (Lwa) | WKDLDERKQREKIEARYNKQIESKI |
| NO: | C2c2 amino | YKEFFRLKNKKRIEKEEDQNIKSLY |
| 25 | acid sequence | FFIKELYLNEKNEEWELKNINLEIL |
| | | DDKERVIKGYKFKEDVYFFKEGYKE |
| | | YYLRILFNNLIEKVQNENREKVRKN |
| | | KEFLDLKEIFKKYKNRKIDLLLKSI |
| | | NNNKINLEYKKENVNEEIYGINPTN |
| | | DREMTFYELLKEIIEKKDEQKSILE |
| | | EKLDNFDITNFLENIEKIFNEETEI |
| | | NIIKGKVLNELREYIKEKEENNSDN |
| | | KLKQIYNLELKKYIENNFSYKKQKS |
| | | KSKNGKNDYLYLNFLKKIMFIEEVD |
| | | EKKEINKEKFKNKINSNFKNLFVQH |
| | | ILDYGKLLYYKENDEYIKNTGQLET |
| | | KDLEYIKTKETLIRKMAVLVSFAAN |
| | | SYYNLFGRVSGDILGTEVVKSSKTN |
| | | VIKVGSHIFKEKMLNYFFDFEIFDA |
| | | NKIVEILESISYSIYNVRNGVGHFN |
| | | KLILGKYKKKDINTNKRIEEDLNNN |
| | | EEIKGYFIKKRGEIERKVKEKFLSN |
| | | NLQYYYSKEKIENYFEVYEFEILKR |
| | | KIPFAPNFKRIIKKGEDLFNNKNNK |
| | | KYEYFKNFDKNSAEEKKEFLKTRNF |
| | | LLKELYYNNFYKEFLSKKEEFEKIV |
| | | LEVKEEKKSRGNINNKKSGVSFQSI |
| | | DDYDTKINISDYIASIHKKEMERVE |
| | | KYNEEKQKDTAKYIRDFVEEIFLTG |
| | | FINYLEKDKRLHFLKEEFSILCNNN |
| | | NNVVDFNININEEKIKEFLKENDSK |
| | | TLNLYLFFNMIDSKRISEFRNELVK |
| | | YKQFTKKRLDEEKEFLGIKIELYET |
| | | LIEFVILTREKLDTKKSEEIDAWLV |
| | | DKLYVKDSNEYKEYEEILKLFVDEK |
| | | ILSSKEAPYYATDNKTPILLSNFEK |
| | | TRKYGTQSFLSEIQSNYKYSKVEKE |
| | | NIEDYNKKEEIEQKKKSNIEKLQDL |
| | | KVELHKKWEQNKITEKEIEKYNNTT |
| | | RKINEYNYLKNKEELQNVYLLHEML |
| | | SDLLARNVAFFNKWERDFKFIVIAI |
| | | KQFLRENDKEKVNEFLNPPDNSKGK |
| | | KVYFSVSKYKNTVENIDGIHKNFMN |
| | | LIFLNNKFMNRKIDKMNCAIWVYFR |
| | | NYIAHFLHLHTKNEKISLISQMNLL |
| | | IKLFSYDKKVQNHILKSTKTLLEKY |
| | | NIQINFEISNDKNEVFKYKIKNRLY |
| | | SKKGKMLGKNNKFEILENEFLENVK |
| | | AMLEYSE |
| SEQ | Bergeyella | MENKTSLGNNIYYNPFKPQDKSYFA |
| ID | zoohelcum | GYFNAAMENTDSVFRELGKRLKGKE |
| NO: | Cas13b | YTSENFFDAIFKENISLVEYERYVK |
| 26 | | LLSDYFPMARLLDKKEVPIKERKEN |
| | | FKKNFKGIIKAVRDLRNFYTHKEHG |
| | | EVEITDEIFGVLDEMLKSTVLTVKK |
| | | KKVKTDKTKEILKKSIEKQLDILCQ |
| | | KKLEYLRDTARKIEEKRRNQRERGE |
| | | KELVAPFKYSDKRDDLIAAIYNDAF |
| | | DVYIDKKKDSLKESSKAKYNTKSDP |
| | | QQEEGDLKIPISKNGVVFLLSLFLT |
| | | KQEIHAFKSKIAGFKATVIDEATVS |
| | | EATVSHGKNSICFMATHEIFSHLAY |
| | | KKLKRKVRTAEINYGEAENAEQLSV |
| | | YAKETLMMQMLDELSKVPDVVYQNL |
| | | SEDVQKTFIEDWNEYLKENNGDVGT |
| | | MEEEQVIHPVIRKRYEDKFNYFAIR |
| | | FLDEFAQFPTLRFQVHLGNYLHDSR |
| | | PKENLISDRRIKEKITVFGRLSELE |
| | | HKKALFIKNTETNEDREHYWEIFPN |
| | | PNYDFPKENISVNDKDFPIAGSILD |
| | | REKQPVAGKIGIKVKLLNQQYVSEV |
| | | DKAVKAHQLKQRKASKPSIQNIIEE |
| | | IVPINESNPKEAIVFGGQPTAYLSM |
| | | NDIHSILYEFFDKWEKKKEKLEKKG |
| | | EKELRKEIGKELEKKIVGKIQAQIQ |
| | | QIIDKDTNAKILKPYQDGNSTAIDK |
| | | EKLIKDLKQEQNILQKLKDEQTVRE |
| | | KEYNDFIAYQDKNREINKVRDRNHK |
| | | QYLKDNLKRKYPEAPARKEVLYYRE |
| | | KGKVAVWLANDIKRFMPTDFKNEWK |
| | | GEQHSLLQKSLAYYEQCKEELKNLL |
| | | PEKVFQHLPFKLGGYFQQKYLYQFY |
| | | TCYLDKRLEYISGLVQQAENFKSEN |
| | | KVFKKVENECFKFLKKQNYTHKELD |
| | | ARVQSILGYPIFLERGFMDEKPTII |
| | | KGKTFKGNEALFADWFRYYKEYQNF |
| | | QTFYDTENYPLVELEKKQADRKRKT |
| | | KIYQQKKNDVFTLLMAKHIFKSVFK |
| | | QDSIDQFSLEDLYQSREERLGNQER |
| | | ARQTGERNTNYIWNKTVDLKLCDGK |
| | | ITVENVKLKNVGDFIKYEYDQRVQA |
| | | FLKYEENIEWQAFLIKESKEEENYP |
| | | YVVEREIEQYEKVRREELLKEVHLI |
| | | EEYILEKVKDKEILKKGDNQNFKYY |
| | | ILNGLLKQLKNEDVESYKVFNLNTE |
| | | PEDVNINQLKQEATDLEQKAFVLTY |
| | | IRNKFAHNQLPKKEFWDYCQEKYGK |
| | | IEKEKTYAEYFAEVFKKEKEALIK |
| SEQ | Prevotella | MEDDKKTTDSIRYELKDKHFWAAFL |
| ID | intermedia | NLARHNVYITVNHINKILEEGEINR |
| NO: | Cas13b | DGYETTLKNTWNEIKDINKKDRLSK |
| 27 | | LIIKHFPFLEAATYRLNPTDTTKQK |
| | | EEKQAEAQSLESLRKSFFVFIYKLR |
| | | DLRNHYSHYKHSKSLERPKFEEGLL |
| | | EKMYNIFNASIRLVKEDYQYNKDIN |
| | | PDEDFKHLDRTEEEFNYYFTKDNEG |
| | | NITESGLLFFVSLFLEKKDAIWMQQ |
| | | KLRGFKDNRENKKKMTNEVFCRSRM |
| | | LLPKLRLQSTQTQDWILLDMLNELI |
| | | RCPKSLYERLREEDREKFRVPIEIA |
| | | DEDYDAEQEPFKNTLVRHQDRFPYF |
| | | ALRYFDYNEIFTNLRFQIDLGTYHF |
| | | SIYKKQIGDYKESHHLTHKLYGFER |
| | | IQEFTKQNRPDEWRKFVKTFNSFET |
| | | SKEPYIPETTPHYHLENQKIGIRFR |
| | | NDNDKIWPSLKTNSEKNEKSKYKLD |
| | | KSFQAEAFLSVHELLPMMFYYLLLK |
| | | TENTDNDNEIETKKKENKNDKQEKH |
| | | KIEEIIENKITEIYALYDTFANGEI |
| | | KSIDELEEYCKGKDIEIGHLPKQMI |
| | | AILKDEHKVMATEAERKQEEMLVDV |
| | | QKSLESLDNQINEEIENVERKNSSL |
| | | KSGKIASWLVNDMMRFQPVQKDNEG |
| | | KPLNNSKANSTEYQLLQRTLAFFGS |
| | | EHERLAPYFKQTKLIESSNPHPFLK |
| | | DTEWEKCNNILSFYRSYLEAKKNFL |
| | | ESLKPEDWEKNQYFLKLKEPKTKPK |
| | | TLVQGWKNGFNLPRGIFTEPIRKWF |
| | | MKHRENITVAELKRVGLVAKVIPLF |
| | | FSEEYKDSVQPFYNYHFNVGNINKP |
| | | DEKNFLNCEERRELLRKKKDEFKKM |
| | | TDKEKEENPSYLEFKSWNKFERELR |
| | | LVRNQDIVTWLLCMELFNKKKIKEL |
| | | NVEKIYLKNINTNTTKKEKNTEEKN |
| | | GEEKNIKEKNNILNRIMPMRLPIKV |
| | | YGRENFSKNKKKKIRRNTFFTVYIE |
| | | EKGTKLLKQGNFKALERDRRLGGLF |
| | | SFVKTPSKAESKSNTISKLRVEYEL |
| | | GEYQKARIEIIKDMLALEKTLIDKY |
| | | NSLDTDNFNKMLTDWLELKGEPDKA |
| | | SFQNDVDLLIAVRNAFSHNQYPMRN |
| | | RIAFANINPFSLSSANTSEEKGLGI |
| | | ANQLKDKTHKTIEKIIEIEKPIETK |
| | | E |
| SEQ | Prevotella | MQKQDKLFVDRKKNAIFAFPKYITI |
| ID | buccae | MENKEKPEPIYYELTDKHFWAAFLN |
| NO: | Cas13b | LARHNVYTTINHINRRLEIAELKDD |
| 28 | | GYMMGIKGSWNEQAKKLDKKVRLRD |
| | | LIMKHFPFLEAAAYEMTNSKSPNNK |
| | | EQREKEQSEALSLNNLKNVLFIFLE |
| | | KLQVLRNYYSHYKYSEESPKPIFET |
| | | SLLKNMYKVFDANVRLVKRDYMHHE |
| | | NIDMQRDFTHLNRKKQVGRTKNIID |
| | | SPNFHYHFADKEGNMTIAGLLFFVS |
| | | LFLDKKDAIWMQKKLKGFKDGRNLR |
| | | EQMTNEVFCRSRISLPKLKLENVQT |
| | | KDWMQLDMLNELVRCPKSLYERLRE |
| | | KDRESFKVPFDIFSDDYNAEEEPFK |
| | | NTLVRHQDRFPYFVLRYFDLNEIFE |
| | | QLRFQIDLGTYHFSIYNKRIGDEDE |
| | | VRHLTHHLYGFARIQDFAPQNQPEE |
| | | WRKLVKDLDHFETSQEPYISKTAPH |
| | | YHLENEKIGIKFCSAHNNLFPSLQT |
| | | DKTCNGRSKFNLGTQFTAEAFLSVH |
| | | ELLPMMFYYLLLTKDYSRKESADKV |
| | | EGIIRKEISNIYAIYDAFANNEINS |
| | | IADLTRRLQNTNILQGHLPKQMISI |
| | | LKGRQKDMGKEAERKIGEMIDDTQR |
| | | RLDLLCKQTNQKIRIGKRNAGLLKS |
| | | GKIADWLVNDMMRFQPVQKDQNNIP |
| | | INNSKANSTEYRMLQRALALFGSEN |
| | | FRLKAYFNQMNLVGNDNPHPFLAET |
| | | QWEHQTNILSFYRNYLEARKKYLKG |
| | | LKPQNWKQYQHFLILKVQKTNRNTL |
| | | VTGWKNSFNLPRGIFTQPIREWFEK |
| | | HNNSKRIYDQILSFDRVGFVAKAIP |
| | | LYFAEEYKDNVQPFYDYPFNIGNRL |
| | | KPKKRQFLDKKERVELWQKNKELFK |
| | | NYPSEKKKTDLAYLDFLSWKKFERE |
| | | LRLIKNQDIVTWLMFKELFNMATVE |
| | | GLKIGEIHLRDIDTNTANEESNNIL |
| | | NRIMPMKLPVKTYETDNKGNILKER |
| | | PLATFYIEETETKVLKQGNFKALVK |
| | | DRRLNGLFSFAETTDLNLEEHPISK |
| | | LSVDLELIKYQTTRISIFEMTLGLE |
| | | KKLIDKYSTLPTDSFRNMLERWLQC |
| | | KANRPELKNYVNSLIAVRNAFSHNQ |
| | | YPMYDATLFAEVKKFTLFPSVDTKK |
| | | IELNIAPQLLEIVGKAIKEIEKSEN |
| | | KN |
| SEQ | Porphyromonas | MNTVPASENKGQSRTVEDDPQYFGL |
| ID | gingivalis | YLNLARENLIEVESHVRIKFGKKKL |
| NO: | Cas13b | NEESLKQSLLCDHLLSVDRWTKVYG |
| 29 | | HSRRYLPFLHYFDPDSQIEKDHDSK |
| | | TGVDPDSAQRLIRELYSLLDFLRND |
| | | FSHNRLDGTTFEHLEVSPDISSFIT |
| | | GTYSLACGRAQSRFAVFFKPDDFVL |
| | | AKNRKEQLISVADGKECLTVSGFAF |
| | | FICLFLDREQASGMLSRIRGFKRTD |
| | | ENWARAVHETFCDLCIRHPHDRLES |
| | | SNTKEALLLDMLNELNRCPRILYDM |
| | | LPEEERAQFLPALDENSMNNLSENS |
| | | LDEESRLLWDGSSDWAEALTKRIRH |
| | | QDRFPYLMLRFIEEMDLLKGIRFRV |
| | | DLGEIELDSYSKKVGRNGEYDRTIT |
| | | DHALAFGKLSDFQNEEEVSRMISGE |
| | | ASYPVRFSLFAPRYAIYDNKIGYCH |
| | | TSDPVYPKSKTGEKRALSNPQSMGF |
| | | ISVHDLRKLLLMELLCEGSFSRMQS |
| | | DFLRKANRILDETAEGKLQFSALFP |
| | | EMRHRFIPPQNPKSKDRREKAETTL |
| | | EKYKQEIKGRKDKLNSQLLSAFDMD |
| | | QRQLPSRLLDEWMNIRPASHSVKLR |
| | | TYVKQLNEDCRLRLRKFRKDGDGKA |
| | | RAIPLVGEMATFLSQDIVRMIISEE |
| | | TKKLITSAYYNEMQRSLAQYAGEEN |
| | | RRQFRAIVAELRLLDPSSGHPFLSA |
| | | TMETAHRYTEGFYKCYLEKKREWLA |
| | | KIFYRPEQDENTKRRISVFFVPDGE |
| | | ARKLLPLLIRRRMKEQNDLQDWIRN |
| | | KQAHPIDLPSHLFDSKVMELLKVKD |
| | | GKKKWNEAFKDWWSTKYPDGMQPFY |
| | | GLRRELNIHGKSVSYIPSDGKKFAD |
| | | CYTHLMEKTVRDKKRELRTAGKPVP |
| | | PDLAADIKRSFHRAVNEREFMLRLV |
| | | QEDDRLMLMAINKMMTDREEDILPG |
| | | LKNIDSILDEENQFSLAVHAKVLEK |
| | | EGEGGDNSLSLVPATIEIKSKRKDW |
| | | SKYIRYRYDRRVPGLMSHFPEHKAT |
| | | LDEVKTLLGEYDRCRIKIFDWAFAL |
| | | EGAIMSDRDLKPYLHESSSREGKSG |
| | | EHSTLVKMLVEKKGCLTPDESQYLI |
| | | LIRNKAAHNQFPCAAEMPLIYRDVS |
| | | AKVGSIEGSSAKDLPEGSSLVDSLW |
| | | KKYEMIIRKILPILDPENRFFGKLL |
| | | NNMSQPINDL |
| SEQ | Bacteroides | MESIKNSQKSTGKTLQKDPPYFGLY |
| ID | pyogenes | LNMALLNVRKVENHIRKWLGDVALL |
| NO: | Cas13b | PEKSGFHSLLTTDNLSSAKWTRFYY |
| 30 | | KSRKFLPFLEMFDSDKKSYENRRET |
| | | AECLDTIDRQKISSLLKEVYGKLQD |
| | | IRNAFSHYHIDDQSVKHTALIISSE |
| | | MHRFIENAYSFALQKTRARFTGVFV |
| | | ETDFLQAEEKGDNKKFFAIGGNEGI |
| | | KLKDNALIFLICLFLDREEAFKFLS |
| | | RATGFKSTKEKGFLAVRETFCALCC |
| | | RQPHERLLSVNPREALLMDMLNELN |
| | | RCPDILFEMLDEKDQKSFLPLLGEE |
| | | EQAHILENSLNDELCEAIDDPFEMI |
| | | ASLSKRVRYKNRFPYLMLRYIEEKN |
| | | LLPFIRFRIDLGCLELASYPKKMGE |
| | | ENNYERSVTDHAMAFGRLTDFHNED |
| | | AVLQQITKGITDEVRFSLYAPRYAI |
| | | YNNKIGFVRTSGSDKISFPTLKKKG |
| | | GEGHCVAYTLQNTKSFGFISIYDLR |
| | | KILLLSFLDKDKAKNIVSGLLEQCE |
| | | KHWKDLSENLFDAIRTELQKEFPVP |
| | | LIRYTLPRSKGGKLVSSKLADKQEK |
| | | YESEFERRKEKLTEILSEKDFDLSQ |
| | | IPRRMIDEWLNVLPTSREKKLKGYV |
| | | ETLKLDCRERLRVFEKREKGEHPLP |
| | | PRIGEMATDLAKDIIRMVIDQGVKQ |
| | | RITSAYYSEIQRCLAQYAGDDNRRH |
| | | LDSIIRELRLKDTKNGHPFLGKVLR |
| | | PGLGHTEKLYQRYFEEKKEWLEATF |
| | | YPAASPKRVPRFVNPPTGKQKELPL |
| | | IIRNLMKERPEWRDWKQRKNSHPID |
| | | LPSQLFENEICRLLKDKIGKEPSGK |
| | | LKWNEMFKLYWDKEFPNGMQRFYRC |
| | | KRRVEVFDKVVEYEYSEEGGNYKKY |
| | | YEALIDEVVRQKISSSKEKSKLQVE |
| | | DLTLSVRRVFKRAINEKEYQLRLLC |
| | | EDDRLLFMAVRDLYDWKEAQLDLDK |
| | | IDNMLGEPVSVSQVIQLEGGQPDAV |
| | | IKAECKLKDVSKLMRYCYDGRVKGL |
| | | MPYFANHEATQEQVEMELRHYEDHR |
| | | RRVFNWVFALEKSVLKNEKLRRFYE |
| | | ESQGGCEHRRCIDALRKASLVSEEE |
| | | YEFLVHIRNKSAHNQFPDLEIGKLP |
| | | PNVTSGFCECIWSKYKAIICRIIPF |
| | | IDPERRFFGKLLEQK |
| SEQ | Cas13c | MTEKKSIIFKNKSSVEIVKKDIFSQ |
| ID | | TPDNMIRNYKITLKISEKNPRVVEA |
| NO: | | EIEDLMNSTILKDGRRSARREKSMT |
| 31 | | ERKLIEEKVAENYSLLANCPMEEVD |
| | | SIKIYKIKRFLTYRSNMLLYFASIN |
| | | SFLCEGIKGKDNETEEIWHLKDNDV |
| | | RKEKVKENFKNKLIQSTENYNSSLK |
| | | NQIEEKEKLLRKESKKGAFYRTIIK |
| | | KLQQERIKELSEKSLTEDCEKIIKL |
| | | YSELRHPLMHYDYQYFENLFENKEN |
| | | SELTKNLNLDIFKSLPLVRKMKLNN |
| | | KVNYLEDNDTLFVLQKTKKAKTLYQ |
| | | IYDALCEQKNGFNKFINDFFVSDGE |
| | | ENTVFKQIINEKFQSEMEFLEKRIS |
| | | ESEKKNEKLKKKFDSMKAHFHNINS |
| | | EDTKEAYFWDIHSSSNYKTKYNERK |
| | | NLVNEYTELLGSSKEKKLLREEITQ |
| | | INRKLLKLKQEMEEITKKNSLFRLE |
| | | YKMKIAFGFLFCEFDGNISKFKDEF |
| | | DASNQEKIIQYHKNGEKYLTYFLKE |
| | | EEKEKFNLEKMQKIIQKTEEEDWLL |
| | | PETKNNLFKFYLLTYLLLPYELKGD |
| | | FLGFVKKHYYDIKNVDFMDENQNNI |
| | | QVSQTVEKQEDYFYHKIRLFEKNTK |
| | | KYEIVKYSIVPNEKLKQYFEDLGID |
| | | IKYLTGSVESGEKWLGENLGIDIKY |
| | | LTVEQKSEVSEEKIKKFL |
| SEQ | Cas13c | MEKDKKGEKIDISQEMIEEDLRKIL |
| ID | | ILFSRLRHSMVHYDYEFYQALYSGK |
| NO: | | DFVISDKNNLENRMISQLLDLNIFK |
| 32 | | ELSKVKLIKDKAISNYLDKNTTIHV |
| | | LGQDIKAIRLLDIYRDICGSKNGFN |
| | | KFINTMITISGEEDREYKEKVIEHF |
| | | NKKMENLSTYLEKLEKQDNAKRNNK |
| | | RVYNLLKQKLIEQQKLKEWFGGPYV |
| | | YDIHSSKRYKELYIERKKLVDRHSK |
| | | LFEEGLDEKNKKELTKINDELSKLN |
| | | SEMKEMTKLNSKYRLQYKLQLAFGF |
| | | ILEEFDLNIDTFINNFDKDKDLIIS |
| | | NFMKKRDIYLNRVLDRGDNRLKNII |
| | | KEYKFRDTEDIFCNDRDNNLVKLYI |
| | | LMYILLPVEIRGDFLGFVKKNYYDM |
| | | KHVDFIDKKDKEDKDTFFHDLRLFE |
| | | KNIRKLEITDYSLSSGFLSKEHKVD |
| | | IEKKINDFINRNGAMKLPEDITIEE |
| | | FNKSLILPIMKNYQINFKLLNDIEI |
| | | SALFKIAKDRSITFKQAIDEIKNED |
| | | IKKNSKKNDKNNHKDKNINFTQLMK |
| | | RALHEKIPYKAGMYQIRNNISHIDM |
| | | EQLYIDPLNSYMNSNKNNITISEQI |
| | | EKIIDVCVTGGVTGKELNNNIINDY |
| | | YMKKEKLVFNLKLRKQNDIVSIESQ |
| | | EKNKREEFVFKKYGLDYKDGEINII |
| | | EVIQKVNSLQEELRNIKETSKEKLK |
| | | NKETLFRDISLINGTIRKNINFKIK |
| | | EMVLDIVRMDEIRHINIHIYYKGEN |
| | | YTRSNIIKFKYAIDGENKKYYLKQH |
| | | EINDINLELKDKFVTLICNMDKHPN |
| | | KNKQTINLESNYIQNVKFIIP |
| SEQ | Cas13c | MENKGNNKKIDFDENYNILVAQIKE |
| ID | | YFTKEIENYNNRIDNIIDKKELLKY |
| NO: | | SEKKEESEKNKKLEELNKLKSQKLK |
| 33 | | ILTDEEIKADVIKIIKIFSDLRHSL |
| | | MHYEYKYFENLFENKKNEELAELLN |
| | | LNLFKNLTLLRQMKIENKTNYLEGR |
| | | EEFNIIGKNIKAKEVLGHYNLLAEQ |
| | | KNGFNNFINSFFVQDGTENLEFKKL |
| | | IDEHFVNAKKRLERNIKKSKKLEKE |
| | | LEKMEQHYQRLNCAYVWDIHTSTTY |
| | | KKLYNKRKSLIEEYNKQINEIKDKE |
| | | VITAINVELLRIKKEMEEITKSNSL |
| | | FRLKYKMQIAYAFLEIEFGGNIAKF |
| | | KDEFDCSKMEEVQKYLKKGVKYLKY |
| | | YKDKEAQKNYEFPFEEIFENKDTHN |
| | | EEWLENTSENNLFKFYILTYLLLPM |
| | | EFKGDFLGVVKKHYYDIKNVDFTDE |
| | | SEKELSQVQLDKMIGDSFFHKIRLF |
| | | EKNTKRYEIIKYSILTSDEIKRYFR |
| | | LLELDVPYFEYEKGTDEIGIFNKNI |
| | | ILTIFKYYQIIFRLYNDLEIHGLFN |
| | | ISSDLDKILRDLKSYGNKNINFREF |
| | | LYVIKQNNNSSTEEEYRKIWENLEA |
| | | KYLRLHLLTPEKEEIKTKTKEELEK |
| | | LNEISNLRNGICHLNYKEIIEEILK |
| | | TEISEKNKEATLNEKIRKVINFIKE |
| | | NELDKVELGFNFINDFFMKKEQFMF |
| | | GQIKQVKEGNSDSITTERERKEKNN |
| | | KKLKETYELNCDNLSEFYETSNNLR |
| | | ERANSSSLLEDSAFLKKIGLYKVKN |
| | | NKVNSKVKDEEKRIENIKRKLLKDS |
| | | SDIMGMYKAEVVKKLKEKLILIFKH |
| | | DEEKRIYVTVYDTSKAVPENISKEI |
| | | LVKRNNSKEEYFFEDNNKKYVTEYY |
| | | TLEITETNELKVIPAKKLEGKEFKT |
| | | EKNKENKLMLNNHYCFNVKIIY |
| SEQ | Cas13c | MEEIKHKKNKSSIIRVIVSNYDMTG |
| ID | | IKEIKVLYQKQGGVDTFNLKTIINL |
| NO: | | ESGNLEIISCKPKEREKYRYEFNCK |
| 34 | | TEINTISITKKDKVLKKEIRKYSLE |
| | | LYFKNEKKDTVVAKVTDLLKAPDKI |
| | | EGERNHLRKLSSSTERKLLSKTLCK |
| | | NYSEISKTPIEEIDSIKIYKIKRFL |
| | | NYRSNFLIYFALINDFLCAGVKEDD |
| | | INEVWLIQDKEHTAFLENRIEKITD |
| | | YIFDKLSKDIENKKNQFEKRIKKYK |
| | | TSLEELKTETLEKNKTFYIDSIKTK |
| | | ITNLENKITELSLYNSKESLKEDLI |
| | | KIISIFTNLRHSLMHYDYKSFENLF |
| | | ENIENEELKNLLDLNLFKSIRMSDE |
| | | FKTKNRTNYLDGTESFTIVKKHQNL |
| | | KKLYTYYNNLCDKKNGFNTFINSFF |
| | | VTDGIENTDFKNLIILHFEKEMEEY |
| | | KKSIEYYKIKISNEKNKSKKEKLKE |
| | | KIDLLQSELINMREHKNLLKQIYFF |
| | | DIHNSIKYKELYSERKNLIEQYNLQ |
| | | INGVKDVTAINHINTKLLSLKNKMD |
| | | KITKQNSLYRLKYKLKIAYSFLMIE |
| | | FDGDVSKFKNNFDPTNLEKRVEYLD |
| | | KKEEYLNYTAPKNKFNFAKLEEELQ |
| | | KIQSTSEMGADYLNVSPENNLFKFY |
| | | ILTYIMLPVEFKGDFLGFVKNHYYN |
| | | IKNVDFMDESLLDENEVDSNKLNEK |
| | | IENLKDSSFFNKIRLFEKNIKKYEI |
| | | VKYSVSTQENMKEYFKQLNLDIPYL |
| | | DYKSTDEIGIFNKNMILPIFKYYQN |
| | | VFKLCNDIEIHALLALANKKQQNLE |
| | | YAIYCCSKKNSLNYNELLKTFNRKT |
| | | YQNLSFIRNKIAHLNYKELFSDLFN |
| | | NELDLNTKVRCLIEFSQNNKFDQID |
| | | LGMNFINDYYMKKTRFIFNQRRLRD |
| | | LNVPSKEKIIDGKRKQQNDSNNELL |
| | | KKYGLSRTNIKDIFNKAWY |
| SEQ | Cas13c | MKVRYRKQAQLDTFIIKTEIVNNDI |
| ID | | FIKSIIEKAREKYRYSFLFDGEEKY |
| NO: | | HFKNKSSVEIVKNDIFSQTPDNMIR |
| 35 | | NYKITLKISEKNPRVVEAEIEDLMN |
| | | STILKDGRRSARREKSMTERKLIEE |
| | | KVAENYSLLANCPIEEVDSIKIYKI |
| | | KRFLTYRSNMLLYFASINSFLCEGI |
| | | KGKDNETEEIWHLKDNDVRKEKVKE |
| | | NFKNKLIQSTENYNSSLKNQIEEKE |
| | | KLSSKEFKKGAFYRTIIKKLQQERI |
| | | KELSEKSLTEDCEKIIKLYSELRHP |
| | | LMHYDYQYFENLFENKENSELTKNL |
| | | NLDIFKSLPLVRKMKLNNKVNYLED |
| | | NDTLFVLQKTKKAKTLYQIYDALCE |
| | | QKNGFNKFINDFFVSDGEENTVFKQ |
| | | IINEKFQSEMEFLEKRISESEKKNE |
| | | KLKKKLDSMKAHFRNINSEDTKEAY |
| | | FWDIHSSRNYKTKYNERKNLVNEYT |
| | | KLLGSSKEKKLLREEITKINRQLLK |
| | | LKQEMEEITKKNSLFRLEYKMKIAF |
| | | GFLFCEFDGNISKFKDEFDASNQEK |
| | | IIQYHKNGEKYLTSFLKEEEKEKFN |
| | | LEKMQKIIQKTEEEDWLLPETKNNL |
| | | FKFYLLTYLLLPYELKGDFLGFVKK |
| | | HYYDIKNVDFMDENQNNIQVSQTVE |
| | | KQEDYFYHKIRLFEKNTKKYEIVKY |
| | | SIVPNEKLKQYFEDLGIDIKYLTGS |
| | | VESGEKWLGENLGIDIKYLTVEQKS |
| | | EVSEEKNKKVSLKNNGMFNKTILLF |
| | | VFKYYQIAFKLFNDIELYSLFFLRE |
| | | KSEKPFEVFLEELKDKMIGKQLNFG |
| | | QLLYVVYEVLVKNKDLDKILSKKID |
| | | YRKDKSFSPEIAYLRNFLSHLNYSK |
| | | FLDNFMKINTNKSDENKEVLIPSIK |
| | | IQKMIQFIEKCNLQNQIDFDFNFVN |
| | | DFYMRKEKMFFIQLKQIFPDINSTE |
| | | KQKKSEKEEILRKRYHLINKKNEQI |
| | | KDEHEAQSQLYEKILSLQKIFSCDK |
| | | NNFYRRLKEEKLLFLEKQGKKKISM |
| | | KEIKDKIASDISDLLGILKKEITRD |
| | | IKDKLTEKFRYCEEKLLNISFYNHQ |
| | | DKKKEEGIRVFLIRDKNSDNFKFES |
| | | ILDDGSNKIFISKNGKEITIQCCDK |
| | | VLETLMIEKNTLKISSNGKIISLIP |
| | | HYSYSIDVKY |

In some embodiments, the programmable nuclease is a Type V CRISPR/Cas enzyme. In some embodiments, the Type V CRISPR/Cas enzyme is a programmable Cas12 nuclease. In general, Type V CRISPR/Cas enzymes (e.g., Cas12 or Cas14) lack an HNH domain. A Cas12 nuclease of the present disclosure cleaves a nucleic acids via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. In most instances, the RuvC domain of the Type V Cas effector protein comprises three patrial RuvC domains (RuvC-I, RuvC-II, and RuvC-III, also referred to herein as subdomains). In some instances, the three RuvC subdomains are located within the C-terminal half of the Type V Cas effector protein. In some instances, none of the RuvC subdomains are located at the N terminus of the protein. In some instances, the RuvC subdomains are contiguous. In some instances, the RuvC subdomains are not contiguous with respect to the primary amino acid sequence of the Type V Cas protein, but form a ruvC domain once the protein is produced and folds. In some instances, there are zero to about 50 amino acids between the first and second RuvC subdomains. In some instances, there are zero to about 50 amino acids between the second and third RuvC subdomains. A programmable Cas12 nuclease can be a Cas12a protein, a Cas12b protein, Cas12c protein, Cas12d protein, a Cas12e protein, or a Cas12j protein. In some cases, a suitable Cas12 protein comprises an amino acid sequence having at least 80%, at least 85%, at least 90%, at least 95%, at least 98%, at least 99%, or 100%, amino acid sequence identity to any one of SEQ ID NO: 36-SEQ ID NO: 46. TABLE 2, below, shows exemplary Cas12 programmable nuclease sequences of the present disclosure.

TABLE 2
Cas12 Protein Sequences

| SEQ | | |
|---|---|---|
| ID | | |
| NO | Description | Sequence |
| SEQ | Lachnospiraceae | MSKLEKFTNCYSLSKTLRFKAIPVGKTQENIDNKRLLVED |
| ID | bacterium | EKRAEDYKGVKKLLDRYYLSFINDVLHSIKLKNLNNYISL |
| NO: | ND2006 | FRKKTRTEKENKELENLEINLRKEIAKAFKGNEGYKSLFK |
| 36 | (LbCas12a) | KDIIETILPEFLDDKDEIALVNSFNGFTTAFTGFFDNREN |
| | | MFSEEAKSTSIAFRCINENLTRYISNMDIFEKVDAIFDKH |
| | | EVQEIKEKILNSDYDVEDFFEGEFFNFVLTQEGIDVYNAI |
| | | IGGFVTESGEKIKGLNEYINLYNQKTKQKLPKFKPLYKQV |
| | | LSDRESLSFYGEGYTSDEEVLEVFRNTLNKNSEIFSSIKK |
| | | LEKLFKNFDEYSSAGIFVKNGPAISTISKDIFGEWNVIRD |
| | | KWNAEYDDIHLKKKAVVTEKYEDDRRKSFKKIGSFSLEQL |
| | | QEYADADLSVVEKLKEIIIQKVDEIYKVYGSSEKLFDADF |
| | | VLEKSLKKNDAVVAIMKDLLDSVKSFENYIKAFFGEGKET |
| | | NRDESFYGDFVLAYDILLKVDHIYDAIRNYVTQKPYSKDK |
| | | FKLYFQNPQFMGGWDKDKETDYRATILRYGSKYYLAIMDK |
| | | KYAKCLQKIDKDDVNGNYEKINYKLLPGPNKMLPKVFFSK |
| | | KWMAYYNPSEDIQKIYKNGTFKKGDMFNLNDCHKLIDFFK |
| | | DSISRYPKWSNAYDFNFSETEKYKDIAGFYREVEEQGYKV |
| | | SFESASKKEVDKLVEEGKLYMFQIYNKDFSDKSHGTPNLH |
| | | TMYFKLLFDENNHGQIRLSGGAELFMRRASLKKEELVVHP |
| | | ANSPIANKNPDNPKKTTTLSYDVYKDKRFSEDQYELHIPI |
| | | AINKCPKNIFKINTEVRVLLKHDDNPYVIGIDRGERNLLY |
| | | IVVVDGKGNIVEQYSLNEIINNFNGIRIKTDYHSLLDKKE |
| | | KERFEARQNWTSIENIKELKAGYISQVVHKICELVEKYDA |
| | | VIALEDLNSGFKNSRVKVEKQVYQKFEKMLIDKLNYMVDK |
| | | KSNPCATGGALKGYQITNKFESFKSMSTQNGFIFYIPAWL |
| | | TSKIDPSTGFVNLLKTKYTSIADSKKFISSFDRIMYVPEE |
| | | DLFEFALDYKNFSRTDADYIKKWKLYSYGNRIRIFRNPKK |
| | | NNVFDWEEVCLTSAYKELFNKYGINYQQGDIRALLCEQSD |
| | | KAFYSSFMALMSLMLQMRNSITGRTDVDFLISPVKNSDGI |
| | | FYDSRNYEAQENAILPKNADANGAYNIARKVLWAIGQFKK |
| | | AEDEKLDKVKIAISNKEWLEYAQTSVKH |
| SEQ | Acidaminococcus | MTQFEGFTNLYQVSKTLRFELIPQGKTLKHIQEQGFIEED |
| ID | sp. | KARNDHYKELKPIIDRIYKTYADQCLQLVQLDWENLSAAI |
| NO: | BV316 | DSYRKEKTEETRNALIEEQATYRNAIHDYFIGRTDNLTDA |
| 37 | (AsCas12a) | INKRHAEIYKGLFKAELFNGKVLKQLGTVTTTEHENALLR |
| | | SFDKFTTYFSGFYENRKNVFSAEDISTAIPHRIVQDNFPK |
| | | FKENCHIFTRLITAVPSLREHFENVKKAIGIFVSTSIEEV |
| | | FSFPFYNQLLTQTQIDLYNQLLGGISREAGTEKIKGLNEV |
| | | LNLAIQKNDETAHIIASLPHRFIPLFKQILSDRNTLSFIL |
| | | EEFKSDEEVIQSFCKYKTLLRNENVLETAEALFNELNSID |
| | | LTHIFISHKKLETISSALCDHWDTLRNALYERRISELTGK |
| | | ITKSAKEKVQRSLKHEDINLQEIISAAGKELSEAFKQKTS |
| | | EILSHAHAALDQPLPTTLKKQEEKEILKSQLDSLLGLYHL |
| | | LDWFAVDESNEVDPEFSARLTGIKLEMEPSLSFYNKARNY |
| | | ATKKPYSVEKFKLNFQMPTLASGWDVNKEKNNGAILFVKN |
| | | GLYYLGIMPKQKGRYKALSFEPTEKTSEGFDKMYYDYFPD |
| | | AAKMIPKCSTQLKAVTAHFQTHTTPILLSNNFIEPLEITK |
| | | EIYDLNNPEKEPKKFQTAYAKKTGDQKGYREALCKWIDFT |
| | | RDFLSKYTKTTSIDLSSLRPSSQYKDLGEYYAELNPLLYH |
| | | ISFQRIAEKEIMDAVETGKLYLFQIYNKDFAKGHHGKPNL |
| | | HTLYWTGLFSPENLAKTSIKLNGQAELFYRPKSRMKRMAH |
| | | RLGEKMLNKKLKDQKTPIPDTLYQELYDYVNHRLSHDLSD |
| | | EARALLPNVITKEVSHEIIKDRRFTSDKFFFHVPITLNYQ |
| | | AANSPSKFNQRVNAYLKEHPETPIIGIDRGERNLIYITVI |
| | | DSTGKILEQRSLNTIQQFDYQKKLDNREKERVAARQAWSV |
| | | VGTIKDLKQGYLSQVIHEIVDLMIHYQAVVVLENLNFGFK |
| | | SKRTGIAEKAVYQQFEKMLIDKLNCLVLKDYPAEKVGGVL |
| | | NPYQLTDQFTSFAKMGTQSGFLFYVPAPYTSKIDPLTGFV |
| | | DPFVWKTIKNHESRKHFLEGFDFLHYDVKTGDFILHFKMN |
| | | RNLSFQRGLPGFMPAWDIVFEKNETQFDAKGTPFIAGKRI |
| | | VPVIENHRFTGRYRDLYPANELIALLEEKGIVFRDGSNIL |
| | | PKLLENDDSHAIDTMVALIRSVLQMRNSNAATGEDYINSP |
| | | VRDLNGVCFDSRFQNPEWPMDADANGAYHIALKGQLLLNH |
| | | LKESKDLKLQNGISNQDWLAYIQELRN |
| SEQ | Francisella | MSIYQEFVNKYSLSKTLRFELIPQGKTLENIKARGLILDD |
| ID | novicida | EKRAKDYKKAKQIIDKYHQFFIEEILSSVCISEDLLQNYS |
| NO: | U112 | DVYFKLKKSDDDNLQKDFKSAKDTIKKQISEYIKDSEKFK |
| 38 | (FnCas12a) | NLFNQNLIDAKKGQESDLILWLKQSKDNGIELFKANSDIT |
| | | DIDEALEIIKSFKGWTTYFKGFHENRKNVYSSNDIPTSII |
| | | YRIVDDNLPKFLENKAKYESLKDKAPEAINYEQIKKDLAE |
| | | ELTFDIDYKTSEVNQRVFSLDEVFEIANFNNYLNQSGITK |
| | | FNTIIGGKFVNGENTKRKGINEYINLYSQQINDKTLKKYK |
| | | MSVLFKQILSDTESKSFVIDKLEDDSDVVTTMQSFYEQIA |
| | | AFKTVEEKSIKETLSLLFDDLKAQKLDLSKIYFKNDKSLT |
| | | DLSQQVFDDYSVIGTAVLEYITQQIAPKNLDNPSKKEQEL |
| | | IAKKTEKAKYLSLETIKLALEEFNKHRDIDKQCRFEEILA |
| | | NFAAIPMIFDEIAQNKDNLAQISIKYQNQGKKDLLQASAE |
| | | DDVKAIKDLLDQTNNLLHKLKIFHISQSEDKANILDKDEH |
| | | FYLVFEECYFELANIVPLYNKIRNYITQKPYSDEKFKLNF |
| | | ENSTLANGWDKNKEPDNTAILFIKDDKYYLGVMNKKNNKI |
| | | FDDKAIKENKGEGYKKIVYKLLPGANKMLPKVFFSAKSIK |
| | | FYNPSEDILRIRNHSTHTKNGSPQKGYEKFEFNIEDCRKF |
| | | IDFYKQSISKHPEWKDFGFRFSDTQRYNSIDEFYREVENQ |
| | | GYKLTFENISESYIDSVVNQGKLYLFQIYNKDFSAYSKGR |
| | | PNLHTLYWKALFDERNLQDVVYKLNGEAELFYRKQSIPKK |
| | | ITHPAKEAIANKNKDNPKKESVFEYDLIKDKRFTEDKFFF |
| | | HCPITINFKSSGANKFNDEINLLLKEKANDVHILSIDRGE |
| | | RHLAYYTLVDGKGNIIKQDTFNIIGNDRMKTNYHDKLAAI |
| | | EKDRDSARKDWKKINNIKEMKEGYLSQVVHEIAKLVIEYN |
| | | AIVVFEDLNFGFKRGRFKVEKQVYQKLEKMLIEKLNYLVF |
| | | KDNEFDKTGGVLRAYQLTAPFETFKKMGKQTGIIYYVPAG |
| | | FTSKICPVTGFVNQLYPKYESVSKSQEFFSKFDKICYNLD |
| | | KGYFEFSFDYKNFGDKAAKGKWTIASFGSRLINFRNSDKN |
| | | HNWDTREVYPTKELEKLLKDYSIEYGHGECIKAAICGESD |
| | | KKFFAKLTSVLNTILQMRNSKTGTELDYLISPVADVNGNF |
| | | FDSRQAPKNMPQDADANGAYHIGLKGLMLLGRIKNNQEGK |
| | | KLNLVIKNEEYFEFVQNRNN |
| SEQ | Porphyromonas | MKTQHFFEDFTSLYSLSKTIRFELKPIGKTLENIKKNGLI |
| ID | macacae | RRDEQRLDDYEKLKKVIDEYHEDFIANILSSFSFSEEILQ |
| NO: | (PmCas12a) | SYIQNLSESEARAKIEKTMRDTLAKAFSEDERYKSIFKKE |
| 39 | Moraxella | LVKKDIPVWCPAYKSLCKKFDNFTTSLVPFHENRKNLYTS |
| | | NEITASIPYRIVHVNLPKFIQNIEALCELQKKMGADLYLE |
| | | MMENLRNVWPSFVKTPDDLCNLKTYNHLMVQSSISEYNRF |
| | | VGGYSTEDGTKHQGINEWINIYRQRNKEMRLPGLVFLHKQ |
| | | ILAKVDSSSFISDTLENDDQVFCVLRQFRKLFWNTVSSKE |
| | | DDAASLKDLFCGLSGYDPEAIYVSDAHLATISKNIFDRWN |
| | | YISDAIRRKTEVLMPRKKESVERYAEKISKQIKKRQSYSL |
| | | AELDDLLAHYSEESLPAGFSLLSYFTSLGGQKYLVSDGEV |
| | | ILYEEGSNIWDEVLIAFRDLQVILDKDFTEKKLGKDEEAV |
| | | SVIKKALDSALRLRKFFDLLSGTGAEIRRDSSFYALYTDR |
| | | MDKLKGLLKMYDKVRNYLTKKPYSIEKFKLHFDNPSLLSG |
| | | WDKNKELNNLSVIFRQNGYYYLGIMTPKGKNLFKTLPKLG |
| | | AEEMFYEKMEYKQIAEPMLMLPKVFFPKKTKPAFAPDQSV |
| | | VDIYNKKTFKTGQKGFNKKDLYRLIDFYKEALTVHEWKLF |
| | | NFSFSPTEQYRNIGEFFDEVREQAYKVSMVNVPASYIDEA |
| | | VENGKLYLFQIYNKDFSPYSKGIPNLHTLYWKALFSEQNQ |
| | | SRVYKLCGGGELFYRKASLHMQDTTVHPKGISIHKKNLNK |
| | | KGETSLFNYDLVKDKRFTEDKFFFHVPISINYKNKKITNV |
| | | NQMVRDYIAQNDDLQIIGIDRGERNLLYISRIDTRGNLLE |
| | | QFSLNVIESDKGDLRTDYQKILGDREQERLRRRQEWKSIE |
| | | SIKDLKDGYMSQVVHKICNMVVEHKAIVVLENLNLSFMKG |
| | | RKKVEKSVYEKFERMLVDKLNYLVVDKKNLSNEPGGLYAA |
| | | YQLTNPLFSFEELHRYPQSGILFFVDPWNTSLTDPSTGFV |
| | | NLLGRINYTNVGDARKFFDRFNAIRYDGKGNILFDLDLSR |
| | | FDVRVETQRKLWTLTTFGSRIAKSKKSGKWMVERIENLSL |
| | | CFLELFEQFNIGYRVEKDLKKAILSQDRKEFYVRLIYLFN |
| | | LMMQIRNSDGEEDYILSPALNEKNLQFDSRLIEAKDLPVD |
| | | ADANGAYNVARKGLMVVQRIKRGDHESIHRIGRAQWLRYV |
| | | QEGIVE |
| SEQ | bovoculi | MLFQDFTHLYPLSKTVRFELKPIDRTLEHIHAKNFLSQDE |
| ID | 237 | TMADMHQKVKVILDDYHRDFIADMMGEVKLTKLAEFYDVY |
| NO: | (MbCas12a) | LKFRKNPKDDELQKQLKDLQAVLRKEIVKPIGNGGKYKAG |
| 40 | | YDRLFGAKLFKDGKELGDLAKFVIAQEGESSPKLAHLAHF |
| | | EKFSTYFTGFHDNRKNMYSDEDKHTAIAYRLIHENLPRFI |
| | | DNLQILTTIKQKHSALYDQIINELTASGLDVSLASHLDGY |
| | | HKLLTQEGITAYNTLLGGISGEAGSPKIQGINELINSHHN |
| | | QHCHKSERIAKLRPLHKQILSDGMSVSFLPSKFADDSEMC |
| | | QAVNEFYRHYADVFAKVQSLFDGFDDHQKDGIYVEHKNLN |
| | | ELSKQAFGDFALLGRVLDGYYVDVVNPEFNERFAKAKTDN |
| | | AKAKLTKEKDKFIKGVHSLASLEQAIEHYTARHDDESVQA |
| | | GKLGQYFKHGLAGVDNPIQKIHNNHSTIKGFLERERPAGE |
| | | RALPKIKSGKNPEMTQLRQLKELLDNALNVAHFAKLLTTK |
| | | TTLDNQDGNFYGEFGVLYDELAKIPTLYNKVRDYLSQKPF |
| | | STEKYKLNFGNPTLLNGWDLNKEKDNFGVILQKDGCYYLA |
| | | LLDKAHKKVFDNAPNTGKSIYQKMIYKYLEVRKQFPKVFF |
| | | SKEAIAINYHPSKELVEIKDKGRQRSDDERLKLYRFILEC |
| | | LKIHPKYDKKFEGAIGDIQLFKKDKKGREVPISEKDLFDK |
| | | INGIFSSKPKLEMEDFFIGEFKRYNPSQDLVDQYNIYKKI |
| | | DSNDNRKKENFYNNHPKFKKDLVRYYYESMCKHEEWEESF |
| | | EFSKKLQDIGCYVDVNELFTEIETRRLNYKISFCNINADY |
| | | IDELVEQGQLYLFQIYNKDFSPKAHGKPNLHTLYFKALFS |
| | | EDNLADPIYKLNGEAQIFYRKASLDMNETTIHRAGEVLEN |
| | | KNPDNPKKRQFVYDIIKDKRYTQDKFMLHVPITMNFGVQG |
| | | MTIKEFNKKVNQSIQQYDEVNVIGIDRGERHLLYLTVINS |
| | | KGEILEQCSLNDITTASANGTQMTTPYHKILDKREIERLN |
| | | ARVGWGEIETIKELKSGYLSHVVHQISQLMLKYNAIVVLE |
| | | DLNFGFKRGRFKVEKQIYQNFENALIKKLNHLVLKDKADD |
| | | EIGSYKNALQLTNNFTDLKSIGKQTGFLFYVPAWNTSKID |
| | | PETGFVDLLKPRYENIAQSQAFFGKFDKICYNADKDYFEF |
| | | HIDYAKFTDKAKNSRQIWTICSHGDKRYVYDKTANQNKGA |
| | | AKGINVNDELKSLFARHHINEKQPNLVMDICQNNDKEFHK |
| | | SLMYLLKTLLALRYSNASSDEDFILSPVANDEGVFFNSAL |
| | | ADDTQPQNADANGAYHIALKGLWLLNELKNSDDLNKVKLA |
| | | IDNQTWLNFAQNR |
| SEQ | Moraxella | MGIHGVPAALFQDFTHLYPLSKTVRFELKPIGRTLEHIHA |
| ID | bovoculi | KNFLSQDETMADMYQKVKVILDDYHRDFIADMMGEVKLTK |
| NO: | AAX08_00 | LAEFYDVYLKFRKNPKDDGLQKQLKDLQAVLRKESVKPIG |
| 41 | 205 | SGGKYKTGYDRLFGAKLFKDGKELGDLAKFVIAQEGESSP |
| | (Mb2Cas12a) | KLAHLAHFEKFSTYFTGFHDNRKNMYSDEDKHTAIAYRLI |
| | | HENLPRFIDNLQILTTIKQKHSALYDQIINELTASGLDVS |
| | | LASHLDGYHKLLTQEGITAYNRIIGEVNGYTNKHNQICHK |
| | | SERIAKLRPLHKQILSDGMGVSFLPSKFADDSEMCQAVNE |
| | | FYRHYTDVFAKVQSLFDGFDDHQKDGIYVEHKNLNELSKQ |
| | | AFGDFALLGRVLDGYYVDVVNPEFNERFAKAKTDNAKAKL |
| | | TKEKDKFIKGVHSLASLEQAIEHHTARHDDESVQAGKLGQ |
| | | YFKHGLAGVDNPIQKIHNNHSTIKGFLERERPAGERALPK |
| | | IKSGKNPEMTQLRQLKELLDNALNVAHFAKLLTTKTTLDN |
| | | QDGNFYGEFGVLYDELAKIPTLYNKVRDYLSQKPFSTEKY |
| | | KLNFGNPTLLNGWDLNKEKDNFGVILQKDGCYYLALLDKA |
| | | HKKVFDNAPNTGKNVYQKMVYKLLPGPNKMLPKVFFAKSN |
| | | LDYYNPSAELLDKYAKGTHKKGDNFNLKDCHALIDFFKAG |
| | | INKHPEWQHFGFKFSPTSSYRDLSDFYREVEPQGYQVKFV |
| | | DINADYIDELVEQGKLYLFQIYNKDFSPKAHGKPNLHTLY |
| | | FKALFSEDNLADPIYKLNGEAQIFYRKASLDMNETTIHRA |
| | | GEVLENKNPDNPKKRQFVYDIIKDKRYTQDKFMLHVPITM |
| | | NFGVQGMTIKEFNKKVNQSIQQYDEVNVIGIDRGERHLLY |
| | | LTVINSKGEILEQRSLNDITTASANGTQVTTPYHKILDKR |
| | | EIERLNARVGWGEIETIKELKSGYLSHVVHQINQLMLKYN |
| | | AIVVLEDLNFGFKRGRFKVEKQIYQNFENALIKKLNHLVL |
| | | KDKADDEIGSYKNALQLTNNFTDLKSIGKQTGFLFYVPAW |
| | | NTSKIDPETGFVDLLKPRYENIAQSQAFFGKFDKICYNTD |
| | | KGYFEFHIDYAKFTDKAKNSRQKWAICSHGDKRYVYDKTA |
| | | NQNKGAAKGINVNDELKSLFARYHINDKQPNLVMDICQNN |
| | | DKEFHKSLMCLLKTLLALRYSNASSDEDFILSPVANDEGV |
| | | FFNSALADDTQPQNADANGAYHIALKGLWLLNELKNSDDL |
| | | NKVKLAIDNQTWLNFAQNR |
| SEQ | Moraxella | MGIHGVPAALFQDFTHLYPLSKTVRFELKPIGKTLEHIHA |
| ID | bovoculi | KNFLNQDETMADMYQKVKAILDDYHRDFIADMMGEVKLTK |
| NO: | AAX11_ | LAEFYDVYLKFRKNPKDDGLQKQLKDLQAVLRKEIVKPIG |
| 42 | 00205 | NGGKYKAGYDRLFGAKLFKDGKELGDLAKFVIAQEGESSP |
| | (Mb3Cas12 | KLAHLAHFEKFSTYFTGFHDNRKNMYSDEDKHTAIAYRLI |
| | a) | HENLPRFIDNLQILATIKQKHSALYDQIINELTASGLDVS |
| | | LASHLDGYHKLLTQEGITAYNTLLGGISGEAGSRKIQGIN |
| | | ELINSHHNQHCHKSERIAKLRPLHKQILSDGMGVSFLPSK |
| | | FADDSEVCQAVNEFYRHYADVFAKVQSLFDGFDDYQKDGI |
| | | YVEYKNLNELSKQAFGDFALLGRVLDGYYVDVVNPEFNER |
| | | FAKAKTDNAKAKLTKEKDKFIKGVHSLASLEQAIEHYTAR |
| | | HDDESVQAGKLGQYFKHGLAGVDNPIQKIHNNHSTIKGFL |
| | | ERERPAGERALPKIKSDKSPEIRQLKELLDNALNVAHFAK |
| | | LLTTKTTLHNQDGNFYGEFGALYDELAKIATLYNKVRDYL |
| | | SQKPFSTEKYKLNFGNPTLLNGWDLNKEKDNFGVILQKDG |
| | | CYYLALLDKAHKKVFDNAPNTGKSVYQKMIYKLLPGPNKM |
| | | LPKVFFAKSNLDYYNPSAELLDKYAQGTHKKGDNFNLKDC |
| | | HALIDFFKAGINKHPEWQHFGFKFSPTSSYQDLSDFYREV |
| | | EPQGYQVKFVDINADYINELVEQGQLYLFQIYNKDFSPKA |
| | | HGKPNLHTLYFKALFSEDNLVNPIYKLNGEAEIFYRKASL |
| | | DMNETTIHRAGEVLENKNPDNPKKRQFVYDIIKDKRYTQD |
| | | KFMLHVPITMNFGVQGMTIKEFNKKVNQSIQQYDEVNVIG |
| | | IDRGERHLLYLTVINSKGEILEQRSLNDITTASANGTQMT |
| | | TPYHKILDKREIERLNARVGWGEIETIKELKSGYLSHVVH |
| | | QISQLMLKYNAIVVLEDLNFGFKRGRFKVEKQIYQNFENA |
| | | LIKKLNHLVLKDKADDEIGSYKNALQLTNNFTDLKSIGKQ |
| | | TGFLFYVPAWNTSKIDPETGFVDLLKPRYENIAQSQAFFG |
| | | KFDKICYNADRGYFEFHIDYAKFNDKAKNSRQIWKICSHG |
| | | DKRYVYDKTANQNKGATIGVNVNDELKSLFTRYHINDKQP |
| | | NLVMDICQNNDKEFHKSLMYLLKTLLALRYSNASSDEDFI |
| | | LSPVANDEGVFFNSALADDTQPQNADANGAYHIALKGLWL |
| | | LNELKNSDDLNKVKLAIDNQTWLNFAQNR |
| SEQ | Thiomicrospira | MGIHGVPAATKTFDSEFFNLYSLQKTVRFELKPVGETASF |
| ID | sp. XS5 | VEDFKNEGLKRVVSEDERRAVDYQKVKEIIDDYHRDFIEE |
| NO: | (TsCas12a) | SLNYFPEQVSKDALEQAFHLYQKLKAAKVEEREKALKEWE |
| 43 | | ALQKKLREKVVKCFSDSNKARFSRIDKKELIKEDLINWLV |
| | | AQNREDDIPTVETFNNFTTYFTGFHENRKNIYSKDDHATA |
| | | ISFRLIHENLPKFFDNVISFNKLKEGFPELKFDKVKEDLE |
| | | VDYDLKHAFEIEYFVNFVTQAGIDQYNYLLGGKTLEDGTK |
| | | KQGMNEQINLFKQQQTRDKARQIPKLIPLFKQILSERTES |
| | | QSFIPKQFESDQELFDSLQKLHNNCQDKFTVLQQAILGLA |
| | | EADLKKVFIKTSDLNALSNTIFGNYSVFSDALNLYKESLK |
| | | TKKAQEAFEKLPAHSIHDLIQYLEQFNSSLDAEKQQSTDT |
| | | VLNYFIKTDELYSRFIKSTSEAFTQVQPLFELEALSSKRR |
| | | PPESEDEGAKGQEGFEQIKRIKAYLDTLMEAVHFAKPLYL |
| | | VKGRKMIEGLDKDQSFYEAFEMAYQELESLIIPIYNKARS |
| | | YLSRKPFKADKFKINFDNNTLLSGWDANKETANASILFKK |
| | | DGLYYLGIMPKGKTFLFDYFVSSEDSEKLKQRRQKTAEEA |
| | | LAQDGESYFEKIRYKLLPGASKMLPKVFFSNKNIGFYNPS |
| | | DDILRIRNTASHTKNGTPQKGHSKVEFNLNDCHKMIDFFK |
| | | SSIQKHPEWGSFGFTFSDTSDFEDMSAFYREVENQGYVIS |
| | | FDKIKETYIQSQVEQGNLYLFQIYNKDFSPYSKGKPNLHT |
| | | LYWKALFEEANLNNVVAKLNGEAEIFFRRHSIKASDKVVH |
| | | PANQAIDNKNPHTEKTQSTFEYDLVKDKRYTQDKFFFHVP |
| | | ISLNFKAQGVSKFNDKVNGFLKGNPDVNIIGIDRGERHLL |
| | | YFTVVNQKGEILVQESLNTLMSDKGHVNDYQQKLDKKEQE |
| | | RDAARKSWTTVENIKELKEGYLSHVVHKLAHLIIKYNAIV |
| | | CLEDLNFGFKRGRFKVEKQVYQKFEKALIDKLNYLVFKEK |
| | | ELGEVGHYLTAYQLTAPFESFKKLGKQSGILFYVPADYTS |
| | | KIDPTTGFVNFLDLRYQSVEKAKQLLSDFNAIRFNSVQNY |
| | | FEFEIDYKKLTPKRKVGTQSKWVICTYGDVRYQNRRNQKG |
| | | HWETEEVNVTEKLKALFASDSKTTTVIDYANDDNLIDVIL |
| | | EQDKASFFKELLWLLKLTMTLRHSKIKSEDDFILSPVKNE |
| | | QGEFYDSRKAGEVWPKDADANGAYHIALKGLWNLQQINQW |
| | | EKGKTLNLAIKNQDWFSFIQEKPYQE |
| SEQ | Butyrivibrio | MGIHGVPAAYYQNLTKKYPVSKTIRNELIPIGKTLENIRK |
| ID | sp. NC3005 | NNILESDVKRKQDYEHVKGIMDEYHKQLINEALDNYMLPS |
| NO: | (BsCas12a) | LNQAAEIYLKKHVDVEDREEFKKTQDLLRREVTGRLKEHE |
| 44 | | NYTKIGKKDILDLLEKLPSISEEDYNALESFRNFYTYFTS |
| | | YNKVRENLYSDEEKSSTVAYRLINENLPKFLDNIKSYAFV |
| | | KAAGVLADCIEEEEQDALFMVETFNMTLTQEGIDMYNYQI |
| | | GKVNSAINLYNQKNHKVEEFKKIPKMKVLYKQILSDREEV |
| | | FIGEFKDDETLLSSIGAYGNVLMTYLKSEKINIFFDALRE |
| | | SEGKNVYVKNDLSKTTMSNIVFGSWSAFDELLNQEYDLAN |
| | | ENKKKDDKYFEKRQKELKKNKSYTLEQMSNLSKEDISPIE |
| | | NYIERISEDIEKICIYNGEFEKIVVNEHDSSRKLSKNIKA |
| | | VKVIKDYLDSIKELEHDIKLINGSGQELEKNLVVYVGQEE |
| | | ALEQLRPVDSLYNLTRNYLTKKPFSTEKVKLNFNKSTLLN |
| | | GWDKNKETDNLGILFFKDGKYYLGIMNTTANKAFVNPPAA |
| | | KTENVFKKVDYKLLPGSNKMLPKVFFAKSNIGYYNPSTEL |
| | | YSNYKKGTHKKGPSFSIDDCHNLIDFFKESIKKHEDWSKF |
| | | GFEFSDTADYRDISEFYREVEKQGYKLTFTDIDESYINDL |
| | | IEKNELYLFQIYNKDFSEYSKGKLNLHTLYFMMLFDQRNL |
| | | DNVVYKLNGEAEVFYRPASIAENELVIHKAGEGIKNKNPN |
| | | RAKVKETSTFSYDIVKDKRYSKYKFTLHIPITMNFGVDEV |
| | | RRENDVINNALRTDDNVNVIGIDRGERNLLYVVVINSEGK |
| | | ILEQISLNSIINKEYDIETNYHALLDEREDDRNKARKDWN |
| | | TIENIKELKTGYLSQVVNVVAKLVLKYNAIICLEDLNFGF |
| | | KRGRQKVEKQVYQKFEKMLIEKLNYLVIDKSREQVSPEKM |
| | | GGALNALQLTSKFKSFAELGKQSGIIYYVPAYLTSKIDPT |
| | | TGFVNLFYIKYENIEKAKQFFDGFDFIRFNKKDDMFEFSF |
| | | DYKSFTQKACGIRSKWIVYTNGERIIKYPNPEKNNLFDEK |
| | | VINVTDEIKGLFKQYRIPYENGEDIKEIIISKAEADFYKR |
| | | LFRLLHQTLQMRNSTSDGTRDYIISPVKNDRGEFFCSEFS |
| | | EGTMPKDADANGAYNIARKGLWVLEQIRQKDEGEKVNLSM |
| | | TNAEWLKYAQLHLL |
| SEQ | AacCas12b | MAVKSIKVKLRLDDMPEIRAGLWKLHKEVNAGVRYYTEWL |
| ID | | SLLRQENLYRRSPNGDGEQECDKTAEECKAELLERLRARQ |
| NO: | | VENGHRGPAGSDDELLQLARQLYELLVPQAIGAKGDAQQI |
| 45 | | ARKFLSPLADKDAVGGLGIAKAGNKPRWVRMREAGEPGWE |
| | | EEKEKAETRKSADRTADVLRALADFGLKPLMRVYTDSEMS |
| | | SVEWKPLRKGQAVRTWDRDMFQQAIERMMSWESWNQRVGQ |
| | | EYAKLVEQKNRFEQKNFVGQEHLVHLVNQLQQDMKEASPG |
| | | LESKEQTAHYVTGRALRGSDKVFEKWGKLAPDAPFDLYDA |
| | | EIKNVQRRNTRRFGSHDLFAKLAEPEYQALWREDASFLTR |
| | | YAVYNSILRKLNHAKMFATFTLPDATAHPIWTRFDKLGGN |
| | | LHQYTFLFNEFGERRHAIRFHKLLKVENGVAREVDDVTVP |
| | | ISMSEQLDNLLPRDPNEPIALYFRDYGAEQHFTGEFGGAK |
| | | IQCRRDQLAHMHRRRGARDVYLNVSVRVQSQSEARGERRP |
| | | PYAAVFRLVGDNHRAFVHFDKLSDYLAEHPDDGKLGSEGL |
| | | LSGLRVMSVDLGLRTSASISVFRVARKDELKPNSKGRVPF |
| | | FFPIKGNDNLVAVHERSQLLKLPGETESKDLRAIREERQR |
| | | TLRQLRTQLAYLRLLVRCGSEDVGRRERSWAKLIEQPVDA |
| | | ANHMTPDWREAFENELQKLKSLHGICSDKEWMDAVYESVR |
| | | RVWRHMGKQVRDWRKDVRSGERPKIRGYAKDVVGGNSIEQ |
| | | IEYLERQYKFLKSWSFFGKVSGQVIRAEKGSRFAITLREH |
| | | IDHAKEDRLKKLADRIIMEALGYVYALDERGKGKWVAKYP |
| | | PCQLILLEELSEYQFNNDRPPSENNQLMQWSHRGVFQELI |
| | | NQAQVHDLLVGTMYAAFSSRFDARTGAPGIRCRRVPARCT |
| | | QEHNPEPFPWWLNKFVVEHTLDACPLRADDLIPTGEGEIF |
| | | VSPFSAEEGDFHQIHADLNAAQNLQQRLWSDFDISQIRLR |
| | | CDWGEVDGELVLIPRLTGKRTADSYSNKVFYTNTGVTYYE |
| | | RERGKKRRKVFAQEKLSEEEAELLVEADEAREKSVVLMRD |
| | | PSGIINRGNWTRQKEFWSMVNQRIEGYLVKQIRSRVPLQD |
| | | SACENTGDI |
| SEQ | Cas12 | MKKIDNFVGCYPVSKTLRFKAIPIGKTQENIEKKRLVEED |
| ID | Variant | EVRAKDYKAVKKLIDRYHREFIEGVLDNVKLDGLEEYYML |
| NO: | | FNKSDREESDNKKIEIMEERFRRVISKSFKNNEEYKKIFS |
| 46 | | KKIIEEILPNYIKDEEEKELVKGFKGFYTAFVGYAQNREN |
| | | MYSDEKKSTAISYRIVNENMPRFITNIKVFEKAKSILDVD |
| | | KINEINEYILNNDYYVDDFFNIDFFNYVLNQKGIDIYNAI |
| | | IGGIVTGDGRKIQGLNECINLYNQENKKIRLPQFKPLYKQ |
| | | ILSESESMSFYIDEIESDDMLIDMLKESLQIDSTINNAID |
| | | DLKVLFNNIFDYDLSGIFINNGLPITTISNDVYGQWSTIS |
| | | DGWNERYDVLSNAKDKESEKYFEKRRKEYKKVKSFSISDL |
| | | QELGGKDLSICKKINEIISEMIDDYKSKIEEIQYLFDIKE |
| | | LEKPLVTDLNKIELIKNSLDGLKRIERYVIPFLGTGKEQN |
| | | RDEVFYGYFIKCIDAIKEIDGVYNKTRNYLTKKPYSKDKF |
| | | KLYFENPQLMGGWDRNKESDYRSTLLRKNGKYYVAIIDKS |
| | | SSNCMMNIEEDENDNYEKINYKLLPGPNKMLPKVFFSKKN |
| | | REYFAPSKEIERIYSTGTFKKDTNFVKKDCENLITFYKDS |
| | | LDRHEDWSKSFDFSFKESSAYRDISEFYRDVEKQGYRVSF |
| | | DLLSSNAVNTLVEEGKLYLFQLYNKDFSEKSHGIPNLHTM |
| | | YFRSLFDDNNKGNIRLNGGAEMFMRRASLNKQDVTVHKAN |
| | | QPIKNKNLLNPKKTTTLPYDVYKDKRFTEDQYEVHIPITM |
| | | NKVPNNPYKINHMVREQLVKDDNPYVIGIDRGERNLIYVV |
| | | VVDGQGHIVEQLSLNEIINENNGISIRTDYHTLLDAKERE |
| | | RDESRKQWKQIENIKELKEGYISQVVHKICELVEKYDAVI |
| | | ALEDLNSGFKNSRVKVEKQVYQKFEKMLITKLNYMVDKKK |
| | | DYNKPGGVLNGYQLTTQFESFSKMGTQNGIMFYIPAWLTS |
| | | KMDPTTGFVDLLKPKYKNKADAQKFFSQFDSIRYDNQEDA |
| | | FVFKVNYTKFPRTDADYNKEWEIYTNGERIRVFRNPKKNN |
| | | EYDYETVNVSERMKELFDSYDLLYDKGELKETICEMEESK |
| | | FFEELIKLFRLTLQMRNSISGRTDVDYLISPVKNSNGYFY |
| | | NSNDYKKEGAKYPKDADANGAYNIARKVLWAIEQFKMADE |
| | | DKLDKTKISIKNQEWLEYAQTHCE |

Alternatively, the Type V CRISPR/Cas enzyme is a programmable Cas14 nuclease. Cas14 proteins may comprise a bilobed structure with distinct amino-terminal and carboxy-terminal domains. The amino- and carboxy-terminal domains may be connected by a flexible linker. The flexible linker may affect the relative conformations of the amino- and carboxyl-terminal domains. The flexible linker may be short, for example less than 10 amino acids, less than 8 amino acids, less than 6 amino acids, less than 5 amino acids, or less than 4 amino acids in length. The flexible linker may be sufficiently long to enable different conformations of the amino- and carboxy-terminal domains among two Cas14 proteins of a Cas14 dimer complex (e.g., the relative orientations of the amino- and carboxy-terminal domains differ between two Cas14 proteins of a Cas14 homodimer complex). The linker domain may comprise a mutation which affects the relative conformations of the amino- and carboxyl-terminal domains. The linker may comprise a mutation which affects Cas14 dimerization. For example, a linker mutation may enhance the stability of a Cas14 dimer.

In some instances, the amino-terminal domain of a Cas14 protein comprises a wedge domain, a recognition domain, a zinc finger domain, or any combination thereof. The wedge domain may comprise a multi-strand β-barrel structure. A multi-strand β-barrel structure may comprise an oligonucleotide/oligosaccharide-binding fold that is structurally comparable to those of some Cas12 proteins. The recognition domain and the zinc finger domain may each (individually or collectively) be inserted between β-barrel strands of the wedge domain. The recognition domain may comprise a 4-α-helix structure, structurally comparable but shorter than those found in some Cas12 proteins. The recognition domain may comprise a binding affinity for a guide nucleic acid or for a guide nucleic acid-target nucleic acid heteroduplex. In some cases, a REC lobe may comprise a binding affinity for a PAM sequence in the target nucleic acid. The amino-terminal may comprise a wedge domain, a recognition domain, and a zinc finger domain. The carboxy-terminal may comprise a RuvC domain, a zinc finger domain, or any combination thereof. The carboxy-terminal may comprise one RuvC and one zinc finger domain.

Cas14 proteins may comprise a RuvC domain or a partial RuvC domain. The RuvC domain may be defined by a single, contiguous sequence, or a set of partial RuvC domains that are not contiguous with respect to the primary amino acid sequence of the Cas14 protein. In some instances, a partial RuvC domain does not have any substrate binding activity or catalytic activity on its own. A Cas14 protein of the present disclosure may include multiple partial RuvC domains, which may combine to generate a RuvC domain with substrate binding or catalytic activity. For example, a Cas14 may include 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A Cas14 protein may comprise a linker loop connecting a carboxy terminal domain of the Cas14 protein with the amino terminal domain of the Cas 14 protein, and wherein the carboxy terminal domain comprises one or more RuvC domains and the amino terminal domain comprises a recognition domain.

Cas14 proteins may comprise a zinc finger domain. In some instances, a carboxy terminal domain of a Cas14 protein comprises a zinc finger domain. In some instances, an amino terminal domain of a Cas14 protein comprises a zinc finger domain. In some instances, the amino terminal domain comprises a wedge domain (e.g., a multi-(3-barrel wedge structure), a zinc finger domain, or any combination thereof. In some cases, the carboxy terminal domain comprises the RuvC domains and a zinc finger domain, and the amino terminal domain comprises a recognition domain, a wedge domain, and a zinc finger domain.

A naturally occurring Cas14 protein functions as an endonuclease that catalyzes cleavage at a specific sequence in a target nucleic acid. A programmable Cas14 nuclease can be a Cas14a protein, a Cas14b protein, a Cas14c protein, a Cas14d protein, a Cas14e protein, a Cas 14f protein, a Cas14g protein, a Cas14h protein, or a Cas14u protein. In some cases, a suitable Cas14 protein comprises an amino acid sequence having at least 80%, at least 85%, at least 90%, at least 95%, at least 98%, at least 99%, or 100%, amino acid sequence identity to any one of SEQ ID NO: 47-SEQ ID NO: 138, which are provide in TABLE 3.

TABLE 3
Cas14 Protein Sequences

| SEQ | |
|---|---|
| ID | |
| NO | Sequence |
| SEQ | MEVQKTVMKTLSLRILRPLYSQEIEKEIKEEKERRKQAGG |
| ID | TGELDGGFYKKLEKKHSEMFSFDRLNLLLNQLQREIAKVY |
| NO: | NHAISELYIATIAQGNKSNKHYISSIVYNRAYGYFYNAYI |
| 47 | ALGICSKVEANFRSNELLTQQSALPTAKSDNFPIVLHKQK |
| | GAEGEDGGFRISTEGSDLIFEIPIPFYEYNGENRKEPYKW |
| | VKKGGQKPVLKLILSTFRRQRNKGWAKDEGTDAEIRKVTE |
| | GKYQVSQIEINRGKKLGEHQKWFANFSIEQPIYERKPNRS |
| | IVGGLDVGIRSPLVCAINNSFSRYSVDSNDVFKFSKQVFA |
| | FRRRLLSKNSLKRKGHGAAHKLEPITEMTEKNDKFRKKII |
| | ERWAKEVTNFFVKNQVGIVQIEDLSTMKDREDHFFNQYLR |
| | GFWPYYQMQTLIENKLKEYGIEVKRVQAKYTSQLCSNPNC |
| | RYWNNYFNFEYRKVNKFPKFKCEKCNLEISADYNAARNLS |
| | TPDIEKFVAKATKGINLPEK |
| SEQ | MEEAKTVSKTLSLRILRPLYSAEIEKEIKEEKERRKQGGK |
| ID | SGELDSGFYKKLEKKHTQMFGWDKLNLMLSQLQRQIARVF |
| NO: | NQSISELYIETVIQGKKSNKHYTSKIVYNRAYSVFYNAYL |
| 48 | ALGITSKVEANFRSTELLMQKSSLPTAKSDNFPILLHKQK |
| | GVEGEEGGFKISADGNDLIFEIPIPFYEYDSANKKEPFKW |
| | IKKGGQKPTIKLILSTFRRQRNKGWAKDEGTDAEIRKVIE |
| | GKYQVSHIEINRGKKLGDHQKWFVNFTIEQPIYERKLDKN |
| | IIGGIDVGIKSPLVCAVNNSFARYSVDSNDVLKFSKQAFA |
| | FRRRLLSKNSLKRSGHGSKNKLDPITRMTEKNDRFRKKII |
| | ERWAKEVTNFFIKNQVGTVQIEDLSTMKDRQDNFFNQYLR |
| | GFWPYYQMQNLIENKLKEYGIETKRIKARYTSQLCSNPSC |
| | RHWNSYFSFDHRKTNNFPKFKCEKCALEISADYNAARNIS |
| | TPDIEKFVAKATKGINLPDKNENVILE |
| SEQ | MAKNTITKTLKLRIVRPYNSAEVEKIVADEKNNREKIALE |
| ID | KNKDKVKEACSKHLKVAAYCTTQVERNACLFCKARKLDDK |
| NO: | FYQKLRGQFPDAVFWQEISEIFRQLQKQAAEIYNQSLIEL |
| 49 | YYEIFIKGKGIANASSVEHYLSDVCYTRAAELFKNAAIAS |
| | GLRSKIKSNFRLKELKNMKSGLPTTKSDNFPIPLVKQKGG |
| | QYTGFEISNHNSDFIIKIPFGRWQVKKEIDKYRPWEKFDF |
| | EQVQKSPKPISLLLSTQRRKRNKGWSKDEGTEAEIKKVMN |
| | GDYQTSYIEVKRGSKIGEKSAWMLNLSIDVPKIDKGVDPS |
| | IIGGIDVGVKSPLVCAINNAFSRYSISDNDLFHFNKKMFA |
| | RRRILLKKNRHKRAGHGAKNKLKPITILTEKSERFRKKLI |
| | ERWACEIADFFIKNKVGTVQMENLESMKRKEDSYFNIRLR |
| | GFWPYAEMQNKIEFKLKQYGIEIRKVAPNNTSKTCSKCGH |
| | LNNYFNFEYRKKNKFPHFKCEKCNFKENADYNAALNISNP |
| | KLKSTKEEP |
| SEQ | MERQKVPQIRKIVRVVPLRILRPKYSDVIENALKKFKEKG |
| ID | DDTNTNDFWRAIRDRDTEFFRKELNFSEDEINQLERDTLF |
| NO: | RVGLDNRVLFSYFDFLQEKLMKDYNKIISKLFINRQSKSS |
| 50 | FENDLTDEEVEELIEKDVTPFYGAYIGKGIKSVIKSNLGG |
| | KFIKSVKIDRETKKVTKLTAINIGLMGLPVAKSDTFPIKI |
| | IKTNPDYITFQKSTKENLQKIEDYETGIEYGDLLVQITIP |
| | WFKNENKDFSLIKTKEAIEYYKLNGVGKKDLLNINLVLTT |
| | YHIRKKKSWQIDGSSQSLVREMANGELEEKWKSFFDTFIK |
| | KYGDEGKSALVKRRVNKKSRAKGEKGRELNLDERIKRLYD |
| | SIKAKSFPSEINLIPENYKWKLHFSIEIPPMVNDIDSNLY |
| | GGIDFGEQNIATLCVKNIEKDDYDFLTIYGNDLLKHAQAS |
| | YARRRIMRVQDEYKARGHGKSRKTKAQEDYSERMQKLRQK |
| | ITERLVKQISDFFLWRNKFHMAVCSLRYEDLNTLYKGESV |
| | KAKRMRQFINKQQLFNGIERKLKDYNSEIYVNSRYPHYTS |
| | RLCSKCGKLNLYFDFLKFRTKNIIIRKNPDGSEIKYMPFF |
| | ICEFCGWKQAGDKNASANIADKDYQDKLNKEKEFCNIRKP |
| | KSKKEDIGEENEEERDYSRRFNRNSFIYNSLKKDNKLNQE |
| | KLFDEWKNQLKRKIDGRNKFEPKEYKDRFSYLFAYYQEII |
| | KNESES |
| SEQ | MVPTELITKTLQLRVIRPLYFEEIEKELAELKEQKEKEFE |
| ID | ETNSLLLESKKIDAKSLKKLKRKARSSAAVEFWKIAKEKY |
| NO: | PDILTKPEMEFIFSEMQKMMARFYNKSMTNIFIEMNNDEK |
| 51 | VNPLSLISKASTEANQVIKCSSISSGLNRKIAGSINKTKF |
| | KQVRDGLISLPTARTETFPISFYKSTANKDEIPISKINLP |
| | SEEEADLTITLPFPFFEIKKEKKGQKAYSYFNIIEKSGRS |
| | NNKIDLLLSTHRRQRRKGWKEEGGTSAEIRRLMEGEFDKE |
| | WEIYLGEAEKSEKAKNDLIKNMTRGKLSKDIKEQLEDIQV |
| | KYFSDNNVESWNDLSKEQKQELSKLRKKKVEELKDWKHVK |
| | EILKTRAKIGWVELKRGKRQRDRNKWFVNITITRPPFINK |
| | ELDDTKFGGIDLGVKVPFVCAVHGSPARLIIKENEILQFN |
| | KMVSARNRQITKDSEQRKGRGKKNKFIKKEIFNERNELFR |
| | KKIIERWANQIVKFFEDQKCATVQIENLESFDRTSYK |
| SEQ | MKSDTKDKKIIIHQTKTLSLRIVKPQSIPMEEFTDLVRYH |
| ID | QMIIFPVYNNGAIDLYKKLFKAKIQKGNEARAIKYFMNKI |
| NO: | VYAPIANTVKNSYIALGYSTKMQSSFSGKRLWDLRFGEAT |
| 52 | PPTIKADFPLPFYNQSGFKVSSENGEFIIGIPFGQYTKKT |
| | VSDIEKKTSFAWDKFTLEDTTKKTLIELLLSTKTRKMNEG |
| | WKNNEGTEAEIKRVMDGTYQVTSLEILQRDDSWFVNFNIA |
| | YDSLKKQPDRDKIAGIHMGITRPLTAVIYNNKYRALSIYP |
| | NTVMHLTQKQLARIKEQRTNSKYATGGHGRNAKVTGTDTL |
| | SEAYRQRRKKIIEDWIASIVKFAINNEIGTIYLEDISNTN |
| | SFFAAREQKLIYLEDISNTNSFLSTYKYPISAISDTLQHK |
| | LEEKAIQVIRKKAYYVNQICSLCGHYNKGFTYQFRRKNKF |
| | PKMKCQGCLEATSTEFNAAANVANPDYEKLLIKHGLLQLK |
| | K |
| SEQ | MSTITRQVRLSPTPEQSRLLMAHCQQYISTVNVLVAAFDS |
| ID | EVLTGKVSTKDFRAALPSAVKNQALRDAQSVFKRSVELGC |
| NO: | LPVLKKPHCQWNNQNWRVEGDQLILPICKDGKTQQERFRC |
| 53 | AAVALEGKAGILRIKKKRGKWIADLTVTQEDAPESSGSAI |
| | MGVDLGIKVPAVAHIGGKGTRFFGNGRSQRSMRRRFYARR |
| | KTLQKAKKLRAVRKSKGKEARWMKTINHQLSRQIVNHAHA |
| | LGVGTIKIEALQGIRKGTTRKSRGAAARKNNRMTNTWSFS |
| | QLTLFITYKAQRQGITVEQVDPAYTSQDCPACRARNGAQD |
| | RTYVCSECGWRGHRDTVGAINISRRAGLSGHRRGATGA |
| SEQ | MIAQKTIKIKLNPTKEQIIKLNSIIEEYIKVSNFTAKKIA |
| ID | EIQESFTDSGLTQGTCSECGKEKTYRKYHLLKKDNKLFCI |
| NO: | TCYKRKYSQFTLQKVEFQNKTGLRNVAKLPKTYYTNAIRF |
| 54 | ASDTFSGFDEIIKKKQNRLNSIQNRLNFWKELLYNPSNRN |
| | EIKIKVVKYAPKTDTREHPHYYSEAEIKGRIKRLEKQLKK |
| | FKMPKYPEFTSETISLQRELYSWKNPDELKISSITDKNES |
| | MNYYGKEYLKRYIDLINSQTPQILLEKENNSFYLCFPITK |
| | NIEMPKIDDTFEPVGIDWGITRNIAVVSILDSKTKKPKFV |
| | KFYSAGYILGKRKHYKSLRKHFGQKKRQDKINKLGTKEDR |
| | FIDSNIHKLAFLIVKEIRNHSNKPIILMENITDNREEAEK |
| | SMRQNILLHSVKSRLQNYIAYKALWNNIPTNLVKPEHTSQ |
| | ICNRCGHQDRENRPKGSKLFKCVKCNYMSNADFNASINIA |
| | RKFYIGEYEPFYKDNEKMKSGVNSISM |
| SEQ | LKLSEQENITTGVKFKLKLDKETSEGLNDYFDEYGKAINF |
| ID | AIKVIQKELAEDRFAGKVRLDENKKPLLNEDGKKIWDFPN |
| NO: | EFCSCGKQVNRYVNGKSLCQECYKNKFTEYGIRKRMYSAK |
| 55 | GRKAEQDINIKNSTNKISKTHFNYAIREAFILDKSIKKQR |
| | KERFRRLREMKKKLQEFIEIRDGNKILCPKIEKQRVERYI |
| | HPSWINKEKKLEDFRGYSMSNVLGKIKILDRNIKREEKSL |
| | KEKGQINFKARRLMLDKSVKFLNDNKISFTISKNLPKEYE |
| | LDLPEKEKRLNWLKEKIKIIKNQKPKYAYLLRKDDNFYLQ |
| | YTLETEFNLKEDYSGIVGIDRGVSHIAVYTFVHNNGKNER |
| | PLFLNSSEILRLKNLQKERDRFLRRKHNKKRKKSNMRNIE |
| | KKIQLILHNYSKQIVDFAKNKNAFIVFEKLEKPKKNRSKM |
| | SKKSQYKLSQFTFKKLSDLVDYKAKREGIKVLYISPEYTS |
| | KECSHCGEKVNTQRPFNGNSSLFKCNKCGVELNADYNASI |
| | NIAKKGLNILNSTN |
| SEQ | MEESIITGVKFKLRIDKETTKKLNEYFDEYGKAINFAVKI |
| ID | IQKELADDRFAGKAKLDQNKNPILDENGKKIYEFPDEFCS |
| NO: | CGKQVNKYVNNKPFCQECYKIRFTENGIRKRMYSAKGRKA |
| 56 | EHKINILNSTNKISKTHFNYAIREAFILDKSIKKQRKKRN |
| | ERLRESKKRLQQFIDMRDGKREICPTIKGQKVDRFIHPSW |
| | ITKDKKLEDFRGYTLSIINSKIKILDRNIKREEKSLKEKG |
| | QIIFKAKRLMLDKSIRFVGDRKVLFTISKTLPKEYELDLP |
| | SKEKRLNWLKEKIEIIKNQKPKYAYLLRKNIESEKKPNYE |
| | YYLQYTLEIKPELKDFYDGAIGIDRGINHIAVCTFISNDG |
| | KVTPPKFFSSGEILRLKNLQKERDRFLLRKHNKNRKKGNM |
| | RVIENKINLILHRYSKQIVDMAKKLNASIVFEELGRIGKS |
| | RTKMKKSQRYKLSLFIFKKLSDLVDYKSRREGIRVTYVPP |
| | EYTSKECSHCGEKVNTQRPFNGNYSLFKCNKCGIQLNSDY |
| | NASINIAKKGLKIPNST |
| SEQ | LWTIVIGDFIEMPKQDLVTTGIKFKLDVDKETRKKLDDYF |
| ID | DEYGKAINFAVKIIQKNLKEDRFAGKIALGEDKKPLLDKD |
| NO: | GKKIYNYPNESCSCGNQVRRYVNAKPFCVDCYKLKFTENG |
| 57 | IRKRMYSARGRKADSDINIKNSTNKISKTHFNYAIREGFI |
| | LDKSLKKQRSKRIKKLLELKRKLQEFIDIRQGQMVLCPKI |
| | KNQRVDKFIHPSWLKRDKKLEEFRGYSLSVVEGKIKIFNR |
| | NILREEDSLRQRGHVNFKANRIMLDKSVRFLDGGKVNFNL |
| | NKGLPKEYLLDLPKKENKLSWLNEKISLIKLQKPKYAYLL |
| | RREGSFFIQYTIENVPKTFSDYLGAIGIDRGISHIAVCTF |
| | VSKNGVNKAPVFFSSGEILKLKSLQKQRDLFLRGKHNKIR |
| | KKSNMRNIDNKINLILHKYSRNIVNLAKSEKAFIVFEKLE |
| | KIKKSRFKMSKSLQYKLSQFTFKKLSDLVEYKAKIEGIKV |
| | DYVPPEYTSKECSHCGEKVDTQRPFNGNSSLFKCNKCRVQ |
| | LNADYNASINIAKKSLNISN |
| SEQ | MSKTTISVKLKIIDLSSEKKEFLDNYFNEYAKATTFCQLR |
| ID | IRRLLRNTHWLGKKEKSSKKWIFESGICDLCGENKELVNE |
| NO: | DRNSGEPAKICKRCYNGRYGNQMIRKLFVSTKKREVQENM |
| 58 | DIRRVAKLNNTHYHRIPEEAFDMIKAADTAEKRRKKNVEY |
| | DKKRQMEFIEMFNDEKKRAARPKKPNERETRYVHISKLES |
| | PSKGYTLNGIKRKIDGMGKKIERAEKGLSRKKIFGYQGNR |
| | IKLDSNWVRFDLAESEITIPSLFKEMKLRITGPTNVHSKS |
| | GQIYFAEWFERINKQPNNYCYLIRKTSSNGKYEYYLQYTY |
| | EAEVEANKEYAGCLGVDIGCSKLAAAVYYDSKNKKAQKPI |
| | EIFTNPIKKIKMRREKLIKLLSRVKVRHRRRKLMQLSKTE |
| | PIIDYTCHKTARKIVEMANTAKAFISMENLETGIKQKQQA |
| | RETKKQKFYRNMFLFRKLSKLIEYKALLKGIKIVYVKPDY |
| | TSQTCSSCGADKEKTERPSQAIFRCLNPTCRYYQRDINAD |
| | FNAAVNIAKKALNNTEVVTTLL |
| SEQ | MARAKNQPYQKLTTTTGIKFKLDLSEEEGKRFDEYFSEYA |
| ID | KAVNFCAKVIYQLRKNLKFAGKKELAAKEWKFEISNCDFC |
| NO: | NKQKEIYYKNIANGQKVCKGCHRTNFSDNAIRKKMIPVKG |
| 59 | RKVESKFNIHNTTKKISGTHRHWAFEDAADIIESMDKQRK |
| | EKQKRLRREKRKLSYFFELFGDPAKRYELPKVGKQRVPRY |
| | LHKIIDKDSLTKKRGYSLSYIKNKIKISERNIERDEKSLR |
| | KASPIAFGARKIKMSKLDPKRAFDLENNVFKIPGKVIKGQ |
| | YKFFGTNVANEHGKKFYKDRISKILAGKPKYFYLLRKKVA |
| | ESDGNPIFEYYVQWSIDTETPAITSYDNILGIDAGITNLA |
| | TTVLIPKNLSAEHCSHCGNNHVKPIFTKFFSGKELKAIKI |
| | KSRKQKYFLRGKHNKLVKIKRIRPIEQKVDGYCHVVSKQI |
| | VEMAKERNSCIALEKLEKPKKSKFRQRRREKYAVSMFVFK |
| | KLATFIKYKAAREGIEIIPVEPEGTSYTCSHCKNAQNNQR |
| | PYFKPNSKKSWTSMFKCGKCGIELNSDYNAAFNIAQKALN |
| | MTSA |
| SEQ | MDEKHFFCSYCNKELKISKNLINKISKGSIREDEAVSKAI |
| ID | SIHNKKEHSLILGIKFKLFIENKLDKKKLNEYFDNYSKAV |
| NO: | TFAARIFDKIRSPYKFIGLKDKNTKKWTFPKAKCVFCLEE |
| 60 | KEVAYANEKDNSKICTECYLKEFGENGIRKKIYSTRGRKV |
| | EPKYNIFNSTKELSSTHYNYAIRDAFQLLDALKKQRQKKL |
| | KSIFNQKLRLKEFEDIFSDPQKRIELSLKPHQREKRYIHL |
| | SKSGQESINRGYTLRFVRGKIKSLTRNIEREEKSLRKKTP |
| | IHFKGNRLMIFPAGIKFDFASNKVKISISKNLPNEFNFSG |
| | TNVKNEHGKSFFKSRIELIKTQKPKYAYVLRKIKREYSKL |
| | RNYEIEKIRLENPNADLCDFYLQYTIETESRNNEEINGII |
| | GIDRGITNLACLVLLKKGDKKPSGVKFYKGNKILGMKIAY |
| | RKHLYLLKGKRNKLRKQRQIRAIEPKINLILHQISKDIVK |
| | IAKEKNFAIALEQLEKPKKARFAQRKKEKYKLALFTFKNL |
| | STLIEYKSKREGIPVIYVPPEKTSQMCSHCAINGDEHVDT |
| | QRPYKKPNAQKPSYSLFKCNKCGIELNADYNAAFNIAQKG |
| | LKTLMLNHSH |
| SEQ | MLQTLLVKLDPSKEQYKMLYETMERFNEACNQIAETVFAI |
| ID | HSANKIEVQKTVYYPIREKFGLSAQLTILAIRKVCEAYKR |
| NO: | DKSIKPEFRLDGALVYDQRVLSWKGLDKVSLVTLQGRQII |
| 61 | PIKFGDYQKARMDRIRGQADLILVKGVFYLCVVVEVSEES |
| | PYDPKGVLGVDLGIKNLAVDSDGEVHSGEQTTNTRERLDS |
| | LKARLQSKGTKSAKRHLKKLSGRMAKFSKDVNHCISKKLV |
| | AKAKGTLMSIALEDLQGIRDRVTVRKAQRRNLHTWNFGLL |
| | RMFVDYKAKIAGVPLVFVDPRNTSRTCPSCGHVAKANRPT |
| | RDEFRCVSCGFAGAADHIAAMNIAFRAEVSQPIVTRFFVQ |
| | SQAPSFRVG |
| SEQ | MDEEPDSAEPNLAPISVKLKLVKLDGEKLAALNDYFNEYA |
| ID | KAVNFCELKMQKIRKNLVNIRGTYLKEKKAWINQTGECCI |
| NO: | CKKIDELRCEDKNPDINGKICKKCYNGRYGNQMIRKLFVS |
| 62 | TNKRAVPKSLDIRKVARLHNTHYHRIPPEAADIIKAIETA |
| | ERKRRNRILFDERRYNELKDALENEEKRVARPKKPKEREV |
| | RYVPISKKDTPSKGYTMNALVRKVSGMAKKIERAKRNLNK |
| | RKKIEYLGRRILLDKNWVRFDFDKSEISIPTMKEFFGEMR |
| | FEITGPSNVMSPNGREYFTKWFDRIKAQPDNYCYLLRKES |
| | EDETDFYLQYTWRPDAHPKKDYTGCLGIDIGGSKLASAVY |
| | FDADKNRAKQPIQIFSNPIGKWKTKRQKVIKVLSKAAVRH |
| | KTKKLESLRNIEPRIDVHCHRIARKIVGMALAANAFISME |
| | NLEGGIREKQKAKETKKQKFSRNMFVFRKLSKLIEYKALM |
| | EGVKVVYIVPDYTSQLCSSCGTNNTKRPKQAIFMCQNTEC |
| | RYFGKNINADFNAAINIAKKALNRKDIVRELS |
| SEQ | MEKNNSEQTSITTGIKFKLKLDKETKEKLNNYFDEYGKAI |
| ID | NFAVRIIQMQLNDDRLAGKYKRDEKGKPILGEDGKKILEI |
| NO: | PNDFCSCGNQVNHYVNGVSFCQECYKKRFSENGIRKRMYS |
| 63 | AKGRKAEQDINIKNSTNKISKTHFNYAIREAFNLDKSIKK |
| | QREKRFKKLKDMKRKLQEFLEIRDGKRVICPKIEKQKVER |
| | YIHPSWINKEKKLEEFRGYSLSIVNSKIKSFDRNIQREEK |
| | SLKEKGQINFKAQRLMLDKSVKFLKDNKVSFTISKELPKT |
| | FELDLPKKEKKLNWLNEKLEIIKNQKPKYAYLLRKENNIF |
| | LQYTLDSIPEIHSEYSGAVGIDRGVSHIAVYTFLDKDGKN |
| | ERPFFLSSSGILRLKNLQKERDKFLRKKHNKIRKKGNMRN |
| | IEQKINLILHEYSKQIVNFAKDKNAFIVFELLEKPKKSRE |
| | RMSKKIQYKLSQFTFKKLSDLVDYKAKREGIKVIYVEPAY |
| | TSKDCSHCGERVNTQRPFNGNFSLFKCNKCGIVLNSDYNA |
| | SLNIARKGLNISAN |
| SEQ | MAEEKFFFCEKCNKDIKIPKNYINKQGAEEKARAKHEHRV |
| ID | HALILGIKFKIYPKKEDISKLNDYFDEYAKAVTFTAKIVD |
| NO: | KLKAPFLFAGKRDKDTSKKKWVFPVDKCSFCKEKTEINYR |
| 64 | TKQGKNICNSCYLTEFGEQGLLEKIYATKGRKVSSSFNLF |
| | NSTKKLTGTHNNYVVKESLQLLDALKKQRSKRLKKLSNTR |
| | RKLKQFEEMFEKEDKRFQLPLKEKQRELRFIHVSQKDRAT |
| | EFKGYTMNKIKSKIKVLRRNIEREQRSLNRKSPVFFRGTR |
| | IRLSPSVQFDDKDNKIKLTLSKELPKEYSFSGLNVANEHG |
| | RKFFAEKLKLIKENKSKYAYLLRRQVNKNNKKPIYDYYLQ |
| | YTVEFLPNIITNYNGILGIDRGINTLACIVLLENKKEKPS |
| | FVKFFSGKGILNLKNKRRKQLYFLKGVHNKYRKQQKIRPI |
| | EPRIDQILHDISKQIIDLAKEKRVAISLEQLEKPQKPKFR |
| | QSRKAKYKLSQFNFKTLSNYIDYKAKKEGIRVIYIAPEMT |
| | SQNCSRCAMKNDLHVNTQRPYKNTSSLFKCNKCGVELNAD |
| | YNAAFNIAQKGLKILNS |
| SEQ | MISLKLKLLPDEEQKKLLDEMFWKWASICTRVGFGRADKE |
| ID | DLKPPKDAEGVWFSLTQLNQANTDINDLREAMKHQKHRLE |
| NO: | YEKNRLEAQRDDTQDALKNPDRREISTKRKDLFRPKASVE |
| 65 | KGFLKLKYHQERYWVRRLKEINKLIERKTKTLIKIEKGRI |
| | KFKATRITLHQGSFKIRFGDKPAFLIKALSGKNQIDAPFV |
| | VVPEQPICGSVVNSKKYLDEITTNFLAYSVNAMLFGLSRS |
| | EEMLLKAKRPEKIKKKEEKLAKKQSAFENKKKELQKLLGR |
| | ELTQQEEAIIEETRNQFFQDFEVKITKQYSELLSKIANEL |
| | KQKNDFLKVNKYPILLRKPLKKAKSKKINNLSPSEWKYYL |
| | QFGVKPLLKQKSRRKSRNVLGIDRGLKHLLAVTVLEPDKK |
| | TFVWNKLYPNPITGWKWRRRKLLRSLKRLKRRIKSQKHET |
| | IHENQTRKKLKSLQGRIDDLLHNISRKIVETAKEYDAVIV |
| | VEDLQSMRQHGRSKGNRLKTLNYALSLFDYANVMQLIKYK |
| | AGIEGIQIYDVKPAGTSQNCAYCLLAQRDSHEYKRSQENS |
| | KIGVCLNPNCQNHKKQIDADLNAARVIASCYALKINDSQP |
| | FGTRKRFKKRTTN |
| SEQ | METLSLKLKLNPSKEQLLVLDKMFWKWASICTRLGLKKAE |
| ID | MSDLEPPKDAEGVWFSKTQLNQANTDVNDLRKAMQHQGKR |
| NO: | IEYELDKVENRRNEIQEMLEKPDRRDISPNRKDLFRPKAA |
| 66 | VEKGYLKLKYHKLGYWSKELKTANKLIERKRKTLAKIDAG |
| | KMKFKPTRISLHTNSFRIKFGEEPKIALSTTSKHEKIELP |
| | LITSLQRPLKTSCAKKSKTYLDAAILNFLAYSTNAALFGL |
| | SRSEEMLLKAKKPEKIEKRDRKLATKRESFDKKLKTLEKL |
| | LERKLSEKEKSVFKRKQTEFFDKFCITLDETYVEALHRIA |
| | EELVSKNKYLEIKKYPVLLRKPESRLRSKKLKNLKPEDWT |
| | YYIQFGFQPLLDTPKPIKTKTVLGIDRGVRHLLAVSIFDP |
| | RTKTFTFNRLYSNPIVDWKWRRRKLLRSIKRLKRRLKSEK |
| | HVHLHENQFKAKLRSLEGRIEDHFHNLSKEIVDLAKENNS |
| | VIVVENLGGMRQHGRGRGKWLKALNYALSHFDYAKVMQLI |
| | KYKAELAGVFVYDVAPAGTSINCAYCLLNDKDASNYTRGK |
| | VINGKKNTKIGECKTCKKEFDADLNAARVIALCYEKRLND |
| | PQPFGTRKQFKPKKP |
| SEQ | MKALKLQLIPTRKQYKILDEMFWKWASLANRVSQKGESKE |
| ID | TLAPKKDIQKIQFNATQLNQIEKDIKDLRGAMKEQQKQKE |
| NO: | RLLLQIQERRSTISEMLNDDNNKERDPHRPLNFRPKGWRK |
| 67 | FHTSKHWVGELSKILRQEDRVKKTIERIVAGKISFKPKRI |
| | GIWSSNYKINFFKRKISINPLNSKGFELTLMTEPTQDLIG |
| | KNGGKSVLNNKRYLDDSIKSLLMFALHSRFFGLNNTDTYL |
| | LGGKINPSLVKYYKKNQDMGEFGREIVEKFERKLKQEINE |
| | QQKKIIMSQIKEQYSNRDSAFNKDYLGLINEFSEVFNQRK |
| | SERAEYLLDSFEDKIKQIKQEIGESLNISDWDFLIDEAKK |
| | AYGYEEGFTEYVYSKRYLEILNKIVKAVLITDIYFDLRKY |
| | PILLRKPLDKIKKISNLKPDEWSYYIQFGYDSINPVQLMS |
| | TDKFLGIDRGLTHLLAYSVFDKEKKEFIINQLEPNPIMGW |
| | KWKLRKVKRSLQHLERRIRAQKMVKLPENQMKKKLKSIEP |
| | KIEVHYHNISRKIVNLAKDYNASIVVESLEGGGLKQHGRK |
| | KNARNRSLNYALSLFDYGKIASLIKYKADLEGVPMYEVLP |
| | AYTSQQCAKCVLEKGSFVDPEIIGYVEDIGIKGSLLDSLF |
| | EGTELSSIQVLKKIKNKIELSARDNHNKEINLILKYNFKG |
| | LVIVRGQDKEEIAEHPIKEINGKFAILDFVYKRGKEKVGK |
| | KGNQKVRYTGNKKVGYCSKHGQVDADLNASRVIALCKYLD |
| | INDPILFGEQRKSFK |
| SEQ | MVTRAIKLKLDPTKNQYKLLNEMFWKWASLANRFSQKGAS |
| ID | KETLAPKDGTQKIQFNATQLNQIKKDVDDLRGAMEKQGKQ |
| NO: | KERLLIQIQERLLTISEILRDDSKKEKDPHRPQNFRPFGW |
| 68 | RRFHTSAYWSSEASKLTRQVDRVRRTIERIKAGKINFKPK |
| | RIGLWSSTYKINFLKKKINISPLKSKSFELDLITEPQQKI |
| | IGKEGGKSVANSKKYLDDSIKSLLIFAIKSRLFGLNNKDK |
| | PLFENIITPNLVRYHKKGQEQENFKKEVIKKFENKLKKEI |
| | SQKQKEIIFSQIERQYENRDATFSEDYLRAISEFSEIFNQ |
| | RKKERAKELLNSFNEKIRQLKKEVNGNISEEDLKILEVEA |
| | EKAYNYENGFIEWEYSEQFLGVLEKIARAVLISDNYFDLK |
| | KYPILIRKPTNKSKKITNLKPEEWDYYIQFGYGLINSPMK |
| | IETKNFMGIDRGLTHLLAYSIFDRDSEKFTINQLELNPIK |
| | GWKWKLRKVKRSLQHLERRMRAQKGVKLPENQMKKRLKSI |
| | EPKIESYYHNLSRKIVNLAKANNASIVVESLEGGGLKQHG |
| | RKKNSRHRALNYALSLFDYGKIASLIKYKSDLEGVPMYEV |
| | LPAYTSQQCAKCVLKKGSFVEPEIIGYIEEIGFKENLLTL |
| | LFEDTGLSSVQVLKKSKNKMTLSARDKEGKMVDLVLKYNF |
| | KGLVISQEKKKEEIVEFPIKEIDGKFAVLDSAYKRGKERI |
| | SKKGNQKLVYTGNKKVGYCSVHGQVDADLNASRVIALCKY |
| | LGINEPIVFGEQRKSFK |
| SEQ | LDLITEPIQPHKSSSLRSKEFLEYQISDFLNFSLHSLFFG |
| ID | LASNEGPLVDFKIYDKIVIPKPEERFPKKESEEGKKLDSF |
| NO: | DKRVEEYYSDKLEKKIERKLNTEEKNVIDREKTRIWGEVN |
| 69 | KLEEIRSIIDEINEIKKQKHISEKSKLLGEKWKKVNNIQE |
| | TLLSQEYVSLISNLSDELTNKKKELLAKKYSKFDDKIKKI |
| | KEDYGLEFDENTIKKEGEKAFLNPDKFSKYQFSSSYLKLI |
| | GEIARSLITYKGFLDLNKYPIIFRKPINKVKKIHNLEPDE |
| | WKYYIQFGYEQINNPKLETENILGIDRGLTHILAYSVFEP |
| | RSSKFILNKLEPNPIEGWKWKLRKLRRSIQNLERRWRAQD |
| | NVKLPENQMKKNLRSIEDKVENLYHNLSRKIVDLAKEKNA |
| | CIVFEKLEGQGMKQHGRKKSDRLRGLNYKLSLFDYGKIAK |
| | LIKYKAEIEGIPIYRIDSAYTSQNCAKCVLESRRFAQPEE |
| | ISCLDDFKEGDNLDKRILEGTGLVEAKIYKKLLKEKKEDF |
| | EIEEDIAMFDTKKVIKENKEKTVILDYVYTRRKEIIGTNH |
| | KKNIKGIAKYTGNTKIGYCMKHGQVDADLNASRTIALCKN |
| | FDINNPEIWK |
| SEQ | MSDESLVSSEDKLAIKIKIVPNAEQAKMLDEMFKKWSSIC |
| ID | NRISRGKEDIETLRPDEGKELQFNSTQLNSATMDVSDLKK |
| NO: | AMARQGERLEAEVSKLRGRYETIDASLRDPSRRHTNPQKP |
| 70 | SSFYPSDWDISGRLTPRFHTARHYSTELRKLKAKEDKMLK |
| | TINKIKNGKIVFKPKRITLWPSSVNMAFKGSRLLLKPFAN |
| | GFEMELPIVISPQKTADGKSQKASAEYMRNALLGLAGYSI |
| | NQLLFGMNRSQKMLANAKKPEKVEKFLEQMKNKDANFDKK |
| | IKALEGKWLLDRKLKESEKSSIAVVRTKFFKSGKVELNED |
| | YLKLLKHMANEILERDGFVNLNKYPILSRKPMKRYKQKNI |
| | DNLKPNMWKYYIQFGYEPIFERKASGKPKNIMGIDRGLTH |
| | LLAVAVFSPDQQKFLFNHLESNPIMHWKWKLRKIRRSIQH |
| | MERRIRAEKNKHIHEAQLKKRLGSIEEKTEQHYHIVSSKI |
| | INWAIEYEAAIVLESLSHMKQRGGKKSVRTRALNYALSLF |
| | DYEKVARLITYKARIRGIPVYDVLPGMTSKTCATCLLNGS |
| | QGAYVRGLETTKAAGKATKRKNMKIGKCMVCNSSENSMID |
| | ADLNAARVIAICKYKNLNDPQPAGSRKVFKRF |
| SEQ | MLALKLKIMPTEKQAEILDAMFWKWASICSRIAKMKKKVS |
| ID | VKENKKELSKKIPSNSDIWFSKTQLCQAEVDVGDHKKALK |
| NO: | NFEKRQESLLDELKYKVKAINEVINDESKREIDPNNPSKF |
| 71 | RIKDSTKKGNLNSPKFFTLKKWQKILQENEKRIKKKESTI |
| | EKLKRGNIFFNPTKISLHEEEYSINFGSSKLLLNCFYKYN |
| | KKSGINSDQLENKFNEFQNGLNIICSPLQPIRGSSKRSFE |
| | FIRNSIINFLMYSLYAKLFGIPRSVKALMKSNKDENKLKL |
| | EEKLKKKKSSFNKTVKEFEKMIGRKLSDNESKILNDESKK |
| | FFEIIKSNNKYIPSEEYLKLLKDISEEIYNSNIDFKPYKY |
| | SILIRKPLSKFKSKKLYNLKPTDYKYYLQLSYEPFSKQLI |
| | ATKTILGIDRGLKHLLAVSVFDPSQNKFVYNKLIKNPVFK |
| | WKKRYHDLKRSIRNRERRIRALTGVHIHENQLIKKLKSMK |
| | NKINVLYHNVSKNIVDLAKKYESTIVLERLENLKQHGRSK |
| | GKRYKKLNYVLSNFDYKKIESLISYKAKKEGVPVSNINPK |
| | YTSKTCAKCLLEVNQLSELKNEYNRDSKNSKIGICNIHGQ |
| | IDADLNAARVIALCYSKNLNEPHFK |
| SEQ | VINLFGYKFALYPNKTQEELLNKHLGECGWLYNKAIEQNE |
| ID | YYKADSNIEEAQKKFELLPDKNSDEAKVLRGNISKDNYVY |
| NO: | RTLVKKKKSEINVQIRKAVVLRPAETIRNLAKVKKKGLSV |
| 72 | GRLKFIPIREWDVLPFKQSDQIRLEENYLILEPYGRLKFK |
| | MHRPLLGKPKTFCIKRTATDRWTISFSTEYDDSNMRKNDG |
| | GQVGIDVGLKTHLRLSNENPDEDPRYPNPKIWKRYDRRLT |
| | ILQRRISKSKKLGKNRTRLRLRLSRLWEKIRNSRADLIQN |
| | ETYEILSENKLIAIEDLNVKGMQEKKDKKGRKGRTRAQEK |
| | GLHRSISDAAFSEFRRVLEYKAKRFGSEVKPVSAIDSSKE |
| | CHNCGNKKGMPLESRIYECPKCGLKIDRDLNSAKVILARA |
| | TGVRPGSNARADTKISATAGASVQTEGTVSEDFRQQMETS |
| | DQKPMQGEGSKEPPMNPEHKSSGRGSKHVNIGCKNKVGLY |
| | NEDENSRSTEKQIMDENRSTTEDMVEIGALHSPVLTT |
| SEQ | MIASIDYEAVSQALIVFEFKAKGKDSQYQAIDEAIRSYRF |
| ID | IRNSCLRYWMDNKKVGKYDLNKYCKVLAKQYPFANKLNSQ |
| NO: | ARQSAAECSWSAISRFYDNCKRKVSGKKGFPKFKKHARSV |
| 73 | EYKTSGWKLSENRKAITFTDKNGIGKLKLKGTYDLHFSQL |
| | EDMKRVRLVRRADGYYVQFCISVDVKVETEPTGKAIGLDV |
| | GIKYFLADSSGNTIENPQFYRKAEKKLNRANRRKSKKYIR |
| | GVKPQSKNYHKARCRYARKHLRVSRQRKEYCKRVAYCVIH |
| | SNDVVAYEDLNVKGMVKNRHLAKSISDVAWSTFRHWLEYF |
| | AIKYGKLTIPVAPHNTSQNCSNCDKKVPKSLSTRTHICHH |
| | CGYSEDRDVNAAKNILKKALSTVGQTGSLKLGEIEPLLVL |
| | EQSCTRKFDL |
| SEQ | LAEENTLHLTLAMSLPLNDLPENRTRSELWRRQWLPQKKL |
| ID | SLLLGVNQSVRKAAADCLRWFEPYQELLWWEPTDPDGKKL |
| NO: | LDKEGRPIKRTAGHMRVLRKLEEIAPFRGYQLGSAVKNGL |
| 74 | RHKVADLLLSYAKRKLDPQFTDKTSYPSIGDQFPIVWTGA |
| | FVCYEQSITGQLYLYLPLFPRGSHQEDITNNYDPDRGPAL |
| | QVFGEKEIARLSRSTSGLLLPLQFDKWGEATFIRGENNPP |
| | TWKATHRRSDKKWLSEVLLREKDFQPKRVELLVRNGRIFV |
| | NVACEIPTKPLLEVENFMGVSFGLEHLVTVVVINRDGNVV |
| | HQRQEPARRYEKTYFARLERLRRRGGPFSQELETFHYRQV |
| | AQIVEEALRFKSVPAVEQVGNIPKGRYNPRLNLRLSYWPF |
| | GKLADLTSYKAVKEGLPKPYSVYSATAKMLCSTCGAANKE |
| | GDQPISLKGPTVYCGNCGTRHNTGFNTALNLARRAQELFV |
| | KGVVAR |
| SEQ | MSQSLLKWHDMAGRDKDASRSLQKSAVEGVLLHLTASHRV |
| ID | ALEMLEKSVSQTVAVTMEAAQQRLVIVLEDDPTKATSRKR |
| NO: | VISADLQFTREEFGSLPNWAQKLASTCPEIATKYADKHIN |
| 75 | SIRIAWGVAKESTNGDAVEQKLQWQIRLLDVTMFLQQLVL |
| | QLADKALLEQIPSSIRGGIGQEVAQQVTSHIQLLDSGTVL |
| | KAELPTISDRNSELARKQWEDAIQTVCTYALPFSRERARI |
| | LDPGKYAAEDPRGDRLINIDPMWARVLKGPTVKSLPLLFV |
| | SGSSIRIVKLTLPRKHAAGHKHTFTATYLVLPVSREWINS |
| | LPGTVQEKVQWWKKPDVLATQELLVGKGALKKSANTLVIP |
| | ISAGKKRFFNHILPALQRGFPLQWQRIVGRSYRRPATHRK |
| | WFAQLTIGYTNPSSLPEMALGIHFGMKDILWWALADKQGN |
| | ILKDGSIPGNSILDFSLQEKGKIERQQKAGKNVAGKKYGK |
| | SLLNATYRVVNGVLEFSKGISAEHASQPIGLGLETIRFVD |
| | KASGSSPVNARHSNWNYGQLSGIFANKAGPAGFSVTEITL |
| | KKAQRDLSDAEQARVLAIEATKRFASRIKRLATKRKDDTL |
| | FV |
| SEQ | VEPVEKERFYYRTYTFRLDGQPRTQNLTTQSGWGLLTKAV |
| ID | LDNTKHYWEIVHHARIANQPIVFENPVIDEQGNPKLNKLG |
| NO: | QPRFWKRPISDIVNQLRALFENQNPYQLGSSLIQGTYWDV |
| 76 | AENLASWYALNKEYLAGTATWGEPSFPEPHPLTEINQWMP |
| | LTFSSGKVVRLLKNASGRYFIGLPILGENNPCYRMRTIEK |
| | LIPCDGKGRVTSGSLILFPLVGIYAQQHRRMTDICESIRT |
| | EKGKLAWAQVSIDYVREVDKRRRMRRTRKSQGWIQGPWQE |
| | VFILRLVLAHKAPKLYKPRCFAGISLGPKTLASCVILDQD |
| | ERVVEKQQWSGSELLSLIHQGEERLRSLREQSKPTWNAAY |
| | RKQLKSLINTQVFTIVTFLRERGAAVRLESIARVRKSTPA |
| | PPVNFLLSHWAYRQITERLKDLAIRNGMPLTHSNGSYGVR |
| | FTCSQCGATNQGIKDPTKYKVDIESETFLCSICSHREIAA |
| | VNTATNLAKQLLDE |
| SEQ | MNDTETSETLTSHRTVCAHLHVVGETGSLPRLVEAALAEL |
| ID | ITLNGRATQALLSLAKNGLVLRRDKEENLIAAELTLPCRK |
| NO: | NKYADVAAKAGEPILATRINNKGKLVTKKWYGEGNSYHIV |
| 77 | RFTPETGMFTVRVFDRYAFDEELLHLHSEVVFGSDLPKGI |
| | KAKTDSLPANFLQAVFTSFLELPFQGFPDIVVKPAMKQAA |
| | EQLLSYVQLEAGENQQAEYPDTNERDPELRLVEWQKSLHE |
| | LSVRTEPFEFVRARDIDYYAETDRRGNRFVNITPEWTKFA |
| | ESPFARRLPLKIPPEFCILLRRKTEGHAKIPNRIYLGLQI |
| | FDGVTPDSTLGVLATAEDGKLFWWHDHLDEFSNLEGKPEP |
| | KLKNKPQLLMVSLEYDREQRFEESVGGDRKICLVTLKETR |
| | NFRRGWNGRILGIHFQHNPVITWALMDHDAEVLEKGFIEG |
| | NAFLGKALDKQALNEYLQKGGKWVGDRSFGNKLKGITHTL |
| | ASLIVRLAREKDAWIALEEISWVQKQSADSVANHEIVEQP |
| | HHSLTR |
| SEQ | MNDTETSETLTSHRTVCAHLHVVGETGSLPRLVEAALAEL |
| ID | ITLNGRATQALLSLAKNGLVLRRDKEENLIAAELTLPCRK |
| NO: | NKYADVAAKAGEPILATRINNKGKLVTKKWYGEGNSYHIV |
| 78 | RFTPETGMFTVRVFDRYAFDEELLHLHSEVVFGSDLPKGI |
| | KAKTDSLPANFLQAVFTSFLELPFQGFPDIVVKPAMKQAA |
| | EQLLSYVQLEAGENQQAEYPDTNERDPELRLVEWQKSLHE |
| | LSVRTEPFEFVRARDIDYYAETDRRGNRFVNITPEWTKFA |
| | ESPFARRLPLKIPPEFCILLRRKTEGHAKIPNRIYLGLQI |
| | FDGVTPDSTLGVLATAEDGKLFWWHDHLDEFSNLEGKPEP |
| | KLKNKPQLLMVSLEYDREQRFEESVGGDRKICLVTLKETR |
| | NFRRGRHGHTRTDRLPAGNTLWRADFATSAEVAAPKWNGR |
| | ILGIHFQHNPVITWALMDHDAEVLEKGFIEGNAFLGKALD |
| | KQALNEYLQKGGKWVGDRSFGNKLKGITHTLASLIVRLAR |
| | EKDAWIALEEISWVQKQSADSVANRRFSMWNYSRLATLIE |
| | WLGTDIATRDCGTAAPLAHKVSDYLTHFTCPECGACRKAG |
| | QKKEIADTVRAGDILTCRKCGFSGPIPDNFIAEFVAKKAL |
| | ERMLKKKPV |
| SEQ | MAKRNFGEKSEALYRAVRFEVRPSKEELSILLAVSEVLRM |
| ID | LFNSALAERQQVFTEFIASLYAELKSASVPEEISEIRKKL |
| NO: | REAYKEHSISLFDQINALTARRVEDEAFASVTRNWQEETL |
| 79 | DALDGAYKSFLSLRRKGDYDAHSPRSRDSGFFQKIPGRSG |
| | FKIGEGRIALSCGAGRKLSFPIPDYQQGRLAETTKLKKFE |
| | LYRDQPNLAKSGRFWISVVYELPKPEATTCQSEQVAFVAL |
| | GASSIGVVSQRGEEVIALWRSDKHWVPKIEAVEERMKRRV |
| | KGSRGWLRLLNSGKRRMHMISSRQHVQDEREIVDYLVRNH |
| | GSHFVVTELVVRSKEGKLADSSKPERGGSLGLNWAAQNTG |
| | SLSRLVRQLEEKVKEHGGSVRKHKLTLTEAPPARGAENKL |
| | WMARKLRESFLKEV |
| SEQ | LAKNDEKELLYQSVKFEIYPDESKIRVLTRVSNILVLVWN |
| ID | SALGERRARFELYIAPLYEELKKFPRKSAESNALRQKIRE |
| NO: | GYKEHIPTFFDQLKKLLTPMRKEDPALLGSVPRAYQEETL |
| 80 | NTLNGSFVSFMTLRRNNDMDAKPPKGRAEDRFHEISGRSG |
| | FKIDGSEFVLSTKEQKLRFPIPNYQLEKLKEAKQIKKFTL |
| | YQSRDRRFWISIAYEIELPDQRPFNPEEVIYIAFGASSIG |
| | VISPEGEKVIDFWRPDKHWKPKIKEVENRMRSCKKGSRAW |
| | KKRAAARRKMYAMTQRQQKLNHREIVASLLRLGFHFVVTE |
| | YTVRSKPGKLADGSNPKRGGAPQGFNWSAQNTGSFGEFIL |
| | WLKQKVKEQGGTVQTFRLVLGQSERPEKRGRDNKIEMVRL |
| | LREKYLESQTIVV |
| SEQ | MAKGKKKEGKPLYRAVRFEIFPTSDQITLFLRVSKNLQQV |
| ID | WNEAWQERQSCYEQFFGSIYERIGQAKKRAQEAGFSEVWE |
| NO: | NEAKKGLNKKLRQQEISMQLVSEKESLLQELSIAFQEHGV |
| 81 | TLYDQINGLTARRIIGEFALIPRNWQEETLDSLDGSFKSF |
| | LALRKNGDPDAKPPRQRVSENSFYKIPGRSGFKVSNGQIY |
| | LSFGKIGQTLTSVIPEFQLKRLETAIKLKKFELCRDERDM |
| | AKPGRFWISVAYEIPKPEKVPVVSKQITYLAIGASRLGVV |
| | SPKGEFCLNLPRSDYHWKPQINALQERLEGVVKGSRKWKK |
| | RMAACTRMFAKLGHQQKQHGQYEVVKKLLRHGVHFVVTEL |
| | KVRSKPGALADASKSDRKGSPTGPNWSAQNTGNIARLIQK |
| | LTDKASEHGGTVIKRNPPLLSLEERQLPDAQRKIFIAKKL |
| | REEFLADQK |
| SEQ | MAKREKKDDVVLRGTKMRIYPTDRQVTLMDMWRRRCISLW |
| ID | NLLLNLETAAYGAKNTRSKLGWRSIWARVVEENHAKALIV |
| NO: | YQHGKCKKDGSFVLKRDGTVKHPPRERFPGDRKILLGLFD |
| 82 | ALRHTLDKGAKCKCNVNQPYALTRAWLDETGHGARTADII |
| | AWLKDFKGECDCTAISTAAKYCPAPPTAELLTKIKRAAPA |
| | DDLPVDQAILLDLFGALRGGLKQKECDHTHARTVAYFEKH |
| | ELAGRAEDILAWLIAHGGTCDCKIVEEAANHCPGPRLFIW |
| | EHELAMIMARLKAEPRTEWIGDLPSHAAQTVVKDLVKALQ |
| | TMLKERAKAAAGDESARKTGFPKFKKQAYAAGSVYFPNTT |
| | MFFDVAAGRVQLPNGCGSMRCEIPRQLVAELLERNLKPGL |
| | VIGAQLGLLGGRIWRQGDRWYLSCQWERPQPTLLPKTGRT |
| | AGVKIAASIVFTTYDNRGQTKEYPMPPADKKLTAVHLVAG |
| | KQNSRALEAQKEKEKKLKARKERLRLGKLEKGHDPNALKP |
| | LKRPRVRRSKLFYKSAARLAACEAIERDRRDGFLHRVTNE |
| | IVHKFDAVSVQKMSVAPMMRRQKQKEKQIESKKNEAKKED |
| | NGAAKKPRNLKPVRKLLRHVAMARGRQFLEYKYNDLRGPG |
| | SVLIADRLEPEVQECSRCGTKNPQMKDGRRLLRCIGVLPD |
| | GTDCDAVLPRNRNAARNAEKRLRKHREAHNA |
| SEQ | MNEVLPIPAVGEDAADTIMRGSKMRIYPSVRQAATMDLWR |
| ID | RRCIQLWNLLLELEQAAYSGENRRTQIGWRSIWATVVEDS |
| NO: | HAEAVRVAREGKKRKDGTFRKAPSGKEIPPLDPAMLAKIQ |
| 83 | RQMNGAVDVDPKTGEVTPAQPRLFMWEHELQKIMARLKQA |
| | PRTHWIDDLPSHAAQSVVKDLIKALQAMLRERKKRASGIG |
| | GRDTGFPKFKKNRYAAGSVYFANTQLRFEAKRGKAGDPDA |
| | VRGEFARVKLPNGVGWMECRMPRHINAAHAYAQATLMGGR |
| | IWRQGENWYLSCQWKMPKPAPLPRAGRTAAIKIAAAIPIT |
| | TVDNRGQTREYAMPPIDRERIAAHAAAGRAQSRALEARKR |
| | RAKKREAYAKKRHAKKLERGIAAKPPGRARIKLSPGFYAA |
| | AAKLAKLEAEDANAREAWLHEITTQIVRNFDVIAVPRMEV |
| | AKLMKKPEPPEEKEEQVKAPWQGKRRSLKAARVMMRRTAM |
| | ALIQTTLKYKAVDLRGPQAYEEIAPLDVTAAACSGCGVLK |
| | PEWKMARAKGREIMRCQEPLPGGKTCNTVLTYTRNSARVI |
| | GRELAVRLAERQKA |
| SEQ | MTTQKTYNFCFYDQRFFELSKEAGEVYSRSLEEFWKIYDE |
| ID | TGVWLSKFDLQKHMRNKLERKLLHSDSFLGAMQQVHANLA |
| NO: | SWKQAKKVVPDACPPRKPKFLQAILFKKSQIKYKNGFLRL |
| 84 | TLGTEKEFLYLKWDINIPLPIYGSVTYSKTRGWKINLCLE |
| | TEVEQKNLSENKYLSIDLGVKRVATIFDGENTITLSGKKF |
| | MGLMHYRNKLNGKTQSRLSHKKKGSNNYKKIQRAKRKTTD |
| | RLLNIQKEMLHKYSSFIVNYAIRNDIGNIIIGDNSSTHDS |
| | PNMRGKTNQKISQNPEQKLKNYIKYKFESISGRVDIVPEP |
| | YTSRKCPHCKNIKKSSPKGRTYKCKKCGFIFDRDGVGAIN |
| | IYNENVSFGQIISPGRIRSLTEPIGMKFHNEIYFKSYVAA |
| SEQ | MSVRSFQARVECDKQTMEHLWRTHKVFNERLPEIIKILFK |
| ID | MKRGECGQNDKQKSLYKSISQSILEANAQNADYLLNSVSI |
| NO: | KGWKPGTAKKYRNASFTWADDAAKLSSQGIHVYDKKQVLG |
| 85 | DLPGMMSQMVCRQSVEAISGHIELTKKWEKEHNEWLKEKE |
| | KWESEDEHKKYLDLREKFEQFEQSIGGKITKRRGRWHLYL |
| | KWLSDNPDFAAWRGNKAVINPLSEKAQIRINKAKPNKKNS |
| | VERDEFFKANPEMKALDNLHGYYERNFVRRRKTKKNPDGF |
| | DHKPTFTLPHPTIHPRWFVFNKPKTNPEGYRKLILPKKAG |
| | DLGSLEMRLLTGEKNKGNYPDDWISVKFKADPRLSLIRPV |
| | KGRRVVRKGKEQGQTKETDSYEFFDKHLKKWRPAKLSGVK |
| | LIFPDKTPKAAYLYFTCDIPDEPLTETAKKIQWLETGDVT |
| | KKGKKRKKKVLPHGLVSCAVDLSMRRGTTGFATLCRYENG |
| | KIHILRSRNLWVGYKEGKGCHPYRWTEGPDLGHIAKHKRE |
| | IRILRSKRGKPVKGEESHIDLQKHIDYMGEDRFKKAARTI |
| | VNFALNTENAASKNGFYPRADVLLLENLEGLIPDAEKERG |
| | INRALAGWNRRHLVERVIEMAKDAGFKRRVFEIPPYGTSQ |
| | VCSKCGALGRRYSIIRENNRREIRFGYVEKLFACPNCGYC |
| | ANADHNASVNLNRRFLIEDSFKSYYDWKRLSEKKQKEEIE |
| | TIESKLMDKLCAMHKISRGSISK |
| SEQ | MHLWRTHCVFNQRLPALLKRLFAMRRGEVGGNEAQRQVYQ |
| ID | RVAQFVLARDAKDSVDLLNAVSLRKRSANSAFKKKATISC |
| NO: | NGQAREVTGEEVFAEAVALASKGVFAYDKDDMRAGLPDSL |
| 86 | FQPLTRDAVACMRSHEELVATWKKEYREWRDRKSEWEAEP |
| | EHALYLNLRPKFEEGEAARGGRFRKRAERDHAYLDWLEAN |
| | PQLAAWRRKAPPAVVPIDEAGKRRIARAKAWKQASVRAEE |
| | FWKRNPELHALHKIHVQYLREFVRPRRTRRNKRREGFKQR |
| | PTFTMPDPVRHPRWCLFNAPQTSPQGYRLLRLPQSRRTVG |
| | SVELRLLTGPSDGAGFPDAWVNVRFKADPRLAQLRPVKVP |
| | RTVTRGKNKGAKVEADGFRYYDDQLLIERDAQVSGVKLLF |
| | RDIRMAPFADKPIEDRLLSATPYLVFAVEIKDEARTERAK |
| | AIRFDETSELTKSGKKRKTLPAGLVSVAVDLDTRGVGFLT |
| | RAVIGVPEIQQTHHGVRLLQSRYVAVGQVEARASGEAEWS |
| | PGPDLAHIARHKREIRRLRQLRGKPVKGERSHVRLQAHID |
| | RMGEDRFKKAARKIVNEALRGSNPAAGDPYTRADVLLYES |
| | LETLLPDAERERGINRALLRWNRAKLIEHLKRMCDDAGIR |
| | HFPVSPFGTSQVCSKCGALGRRYSLARENGRAVIRFGWVE |
| | RLFACPNPECPGRRPDRPDRPFTCNSDHNASVNLHRVFAL |
| | GDQAVAAFRALAPRDSPARTLAVKRVEDTLRPQLMRVHKL |
| | ADAGVDSPF |
| SEQ | MATLVYRYGVRAHGSARQQDAVVSDPAMLEQLRLGHELRN |
| ID | ALVGVQHRYEDGKRAVWSGFASVAAADHRVTTGETAVAEL |
| NO: | EKQARAEHSADRTAATRQGTAESLKAARAAVKQARADRKA |
| 87 | AMAAVAEQAKPKIQALGDDRDAEIKDLYRRFCQDGVLLPR |
| | CGRCAGDLRSDGDCTDCGAAHEPRKLYWATYNAIREDHQT |
| | AVKLVEAKRKAGQPARLRFRRWTGDGTLTVQLQRMHGPAC |
| | RCVTCAEKLTRRARKTDPQAPAVAADPAYPPTDPPRDPAL |
| | LASGQGKWRNVLQLGTWIPPGEWSAMSRAERRRVGRSHIG |
| | WQLGGGRQLTLPVQLHRQMPADADVAMAQLTRVRVGGRHR |
| | MSVALTAKLPDPPQVQGLPPVALHLGWRQRPDGSLRVATW |
| | ACPQPLDLPPAVADVVVSHGGRWGEVIMPARWLADAEVPP |
| | RLLGRRDKAMEPVLEALADWLEAHTEACTARMTPALVRRW |
| | RSQGRLAGLTNRWRGQPPTGSAEILTYLEAWRIQDKLLWE |
| | RESHLRRRLAARRDDAWRRVASWLARHAGVLVVDDADIAE |
| | LRRRDDPADTDPTMPASAAQAARARAALAAPGRLRHLATI |
| | TATRDGLGVHTVASAGLTRLHRKCGHQAQPDPRYAASAVV |
| | TCPGCGNGYDQDYNAAMLMLDRQQQP |
| SEQ | MSRVELHRAYKFRLYPTPAQVAELAEWERQLRRLYNLAHS |
| ID | QRLAAMQRHVRPKSPGVLKSECLSCGAVAVAEIGTDGKAK |
| NO: | KTVKHAVGCSVLECRSCGGSPDAEGRTAHTAACSFVDYYR |
| 88 | QGREMTQLLEEDDQLARVVCSARQETLRDLEKAWQRWHKM |
| | PGFGKPHFKKRIDSCRIYFSTPKSWAVDLGYLSFTGVASS |
| | VGRIKIRQDRVWPGDAKFSSCHVVRDVDEWYAVFPLTFTK |
| | EIEKPKGGAVGINRGAVHAIADSTGRVVDSPKFYARSLGV |
| | IRHRARLLDRKVPFGRAVKPSPTKYHGLPKADIDAAAARV |
| | NASPGRLVYEARARGSIAAAEAHLAALVLPAPRQTSQLPS |
| | EGRNRERARRFLALAHQRVRRQREWFLHNESAHYAQSYTK |
| | IAIEDWSTKEMTSSEPRDAEEMKRVTRARNRSILDVGWYE |
| | LGRQIAYKSEATGAEFAKVDPGLRETETHVPEAIVRERDV |
| | DVSGMLRGEAGISGTCSRCGGLLRASASGHADAECEVCLH |
| | VEVGDVNAAVNVLKRAMFPGAAPPSKEKAKVTIGIKGRKK |
| | KRAA |
| SEQ | MSRVELHRAYKFRLYPTPVQVAELSEWERQLRRLYNLGHE |
| ID | QRLLTLTRHLRPKSPGVLKGECLSCDSTQVQEVGADGRPK |
| NO: | TTVRHAEQCPTLACRSCGALRDAEGRTAHTVACAFVDYYR |
| 89 | QGREMTELLAADDQLARVVCSARQEVLRDLDKAWQRWRKM |
| | PGFGKPRFKRRTDSCRIYFSTPKAWKLEGGHLSFTGAATT |
| | VGAIKMRQDRNWPASVQFSSCHVVRDVDEWYAVFPLTFVA |
| | EVARPKGGAVGINRGAVHAIADSTGRVVDSPRYYARALGV |
| | IRHRARLFDRKVPSGHAVKPSPTKYRGLSAIEVDRVARAT |
| | GFTPGRVVTEALNRGGVAYAECALAAIAVLGHGPERPLTS |
| | DGRNREKARKFLALAHQRVRRQREWFLHNESAHYARTYSK |
| | IAIEDWSTKEMTASEPQGEETRRVTRSRNRSILDVGWYEL |
| | GRQLAYKTEATGAEFAQVDPGLKETETNVPKAIADARDVD |
| | VSGMLRGEAGISGTCSKCGGLLRAPASGHADAECEICLNV |
| | EVGDVNAAVNVLKRAMFPGDAPPASGEKPKVSIGIKGRQK |
| | KKKAA |
| SEQ | MEAIATGMSPERRVELGILPGSVELKRAYKFRLYPMKVQQ |
| ID | AELSEWERQLRRLYNLAHEQRLAALLRYRDWDFQKGACPS |
| NO: | CRVAVPGVHTAACDHVDYFRQAREMTQLLEVDAQLSRVIC |
| 90 | CARQEVLRDLDKAWQRWRKKLGGRPRFKRRTDSCRIYLST |
| | PKHWEIAGRYLRLSGLASSVGEIRIEQDRAFPEGALLSSC |
| | SIVRDVDEWYACLPLTFTQPIERAPHRSVGLNRGVVHALA |
| | DSDGRVVDSPKFFERALATVQKRSRDLARKVSGSRNAHKA |
| | RIKLAKAHQRVRRQRAAFLHQESAYYSKGFDLVALEDMSV |
| | RKMTATAGEAPEMGRGAQRDLNRGILDVGWYELARQIDYK |
| | RLAHGGELLRVDPGQTTPLACVTEEQPARGISSACAVCGI |
| | PLARPASGNARMRCTACGSSQVGDVNAAENVLTRALSSAP |
| | SGPKSPKASIKIKGRQKRLGTPANRAGEASGGDPPVRGPV |
| | EGGTLAYVVEPVSESQSDT |
| SEQ | MTVRTYKYRAYPTPEQAEALTSWLRFASQLYNAALEHRKN |
| ID | AWGRHDAHGRGFRFWDGDAAPRKKSDPPGRWVYRGGGGAH |
| NO: | ISKNDQGKLLTEFRREHAELLPPGMPALVQHEVLARLERS |
| 91 | MAAFFQRATKGQKAGYPRWRSEHRYDSLTFGLTSPSKERF |
| | DPETGESLGRGKTVGAGTYHNGDLRLTGLGELRILEHRRI |
| | PMGAIPKSVIVRRSGKRWFVSIAMEMPSVEPAASGRPAVG |
| | LDMGVVTWGTAFTADTSAAAALVADLRRMATDPSDCRRLE |
| | ELEREAAQLSEVLAHCRARGLDPARPRRCPKELTKLYRRS |
| | LHRLGELDRACARIRRRLQAAHDIAEPVPDEAGSAVLIEG |
| | SNAGMRHARRVARTQRRVARRTRAGHAHSNRRKKAVQAYA |
| | RAKERERSARGDHRHKVSRALVRQFEEISVEALDIKQLTV |
| | APEHNPDPQPDLPAHVQRRRNRGELDAAWGAFFAALDYKA |
| | ADAGGRVARKPAPHTTQECARCGTLVPKPISLRVHRCPAC |
| | GYTAPRTVNSARNVLQRPLEEPGRAGPSGANGRGVPHAVA |
| SEQ | MNCRYRYRIYPTPGQRQSLARLFGCVRVVWNDALFLCRQS |
| ID | EKLPKNSELQKLCITQAKKTEARGWLGQVSAIPLQQSVAD |
| NO: | LGVAFKNFFQSRSGKRKGKKVNPPRVKRRNNRQGARFTRG |
| 92 | GFKVKTSKVYLARIGDIKIKWSRPLPSEPSSVTVIKDCAG |
| | QYFLSFVVEVKPEIKPPKNPSIGIDLGLKTFASCSNGEKI |
| | DSPDYSRLYRKLKRCQRRLAKRQRGSKRRERMRVKVAKLN |
| | AQIRDKRKDFLHKLSTKVVNENQVIALEDLNVGGMLKNRK |
| | LSRAISQAGWYEFRSLCEGKAEKHNRDFRVISRWEPTSQV |
| | CSECGYRWGKIDLSVRSIVCINCGVEHDRDDNASVNIEQA |
| | GLKVGVGHTHDSKRTGSACKTSNGAVCVEPSTHREYVQLT |
| | LFDW |
| SEQ | MKSRWTFRCYPTPEQEQHLARTFGCVRFVWNWALRARTDA |
| ID | FRAGERIGYPATDKALTLLKQQPETVWLNEVSSVCLQQAL |
| NO: | RDLQVAFSNFFDKRAAHPSFKRKEARQSANYTERGFSFDH |
| 93 | ERRILKLAKIGAIKVKWSRKAIPHPSSIRLIRTASGKYFV |
| | SLVVETQPAPMPETGESVGVDFGVARLATLSNGERISNPK |
| | HGAKWQRRLAFYQKRLARATKGSKRRMRIKRHVARIHEKI |
| | GNSRSDTLHKLSTDLVTRFDLICVEDLNLRGMVKNHSLAR |
| | SLHDASIGSAIRMIEEKAERYGKNVVKIDRWFPSSKTCSD |
| | CGHIVEQLPLNVREWTCPECGTTHDRDANAAANILAVGQT |
| | VSAHGGTVRRSRAKASERKSQRSANRQGVNRA |
| SEQ | KEPLNIGKTAKAVFKEIDPTSLNRAANYDASIELNCKECK |
| ID | FKPFKNVKRYEFNFYNNWYRCNPNSCLQSTYKAQVRKVEI |
| NO: | GYEKLKNEILTQMQYYPWFGRLYQNFFHDERDKMTSLDEI |
| 94 | QVIGVQNKVFFNTVEKAWREIIKKRFKDNKETMETIPELK |
| | HAAGHGKRKLSNKSLLRRRFAFVQKSFKFVDNSDVSYRSF |
| | SNNIACVLPSRIGVDLGGVISRNPKREYIPQEISFNAFWK |
| | QHEGLKKGRNIEIQSVQYKGETVKRIEADTGEDKAWGKNR |
| | QRRFTSLILKLVPKQGGKKVWKYPEKRNEGNYEYFPIPIE |
| | FILDSGETSIRFGGDEGEAGKQKHLVIPFNDSKATPLASQ |
| | QTLLENSRFNAEVKSCIGLAIYANYFYGYARNYVISSIYH |
| | KNSKNGQAITAIYLESIAHNYVKAIERQLQNLLLNLRDFS |
| | FMESHKKELKKYFGGDLEGTGGAQKRREKEEKIEKEIEQS |
| | YLPRLIRLSLTKMVTKQVEM |
| SEQ | ELIVNENKDPLNIGKTAKAVFKEIDPTSINRAANYDASIE |
| ID | LACKECKFKPFNNTKRHDFSFYSNWHRCSPNSCLQSTYRA |
| NO | KIRKTEIGYEKLKNEILNQMQYYPWFGRLYQNFFNDQRDK |
| 95 | MTSLDEIQVTGVQNKIFFNTVEKAWREIIKKRFRDNKETM |
| | RTIPDLKNKSGHGSRKLSNKSLLRRRFAFAQKSFKLVDNS |
| | DVSYRAFSNNVACVLPSKIGVDIGGIINKDLKREYIPQEI |
| | TFNVFWKQHDGLKKGRNIEIHSVQYKGEIVKRIEADTGED |
| | KAWGKNRQRRFTSLILKITPKQGGKKIWKFPEKKNASDYE |
| | YFPIPIEFILDNGDASIKFGGEEGEVGKQKHLLIPFNDSK |
| | ATPLSSKQMLLETSRFNAEVKSTIGLALYANYFVSYARNY |
| | VIKSTYHKNSKKGQIVTEIYLESISQNFVRAIQRQLQSLM |
| | LNLKDWGFMQTHKKELKKYFGSDLEGSKGGQKRREKEEKI |
| | EKEIEASYLPRLIRLSLTKSVTKAEEM |
| SEQ | PEEKTSKLKPNSINLAANYDANEKFNCKECKFHPFKNKKR |
| ID | YEFNFYNNLHGCKSCTKSTNNPAVKRIEIGYQKLKFEIKN |
| NO: | QMEAYPWFGRLRINFYSDEKRKMSELNEMQVTGVKNKIFF |
| 96 | DAIECAWREILKKRFRESKETLITIPKLKNKAGHGARKHR |
| | NKKLLIRRRAFMKKNFHFLDNDSISYRSFANNIACVLPSK |
| | VGVDIGGIISPDVGKDIKPVDISLNLMWASKEGIKSGRKV |
| | EIYSTQYDGNMVKKIEAETGEDKSWGKNRKRRQTSLLLSI |
| | PKPSKQVQEFDFKEWPRYKDIEKKVQWRGFPIKIIFDSNH |
| | NSIEFGTYQGGKQKVLPIPFNDSKTTPLGSKMNKLEKLRF |
| | NSKIKSRLGSAIAANKFLEAARTYCVDSLYHEVSSANAIG |
| | KGKIFIEYYLEILSQNYIEAAQKQLQRFIESIEQWFVADP |
| | FQGRLKQYFKDDLKRAKCFLCANREVQTTCYAAVKLHKSC |
| | AEKVKDKNKELAIKERNNKEDAVIKEVEASNYPRVIRLKL |
| | TKTITNKAM |
| SEQ | SESENKIIEQYYAFLYSFRDKYEKPEFKNRGDIKRKLQNK |
| ID | WEDFLKEQNLKNDKKLSNYIFSNRNFRRSYDREEENEEGI |
| NO: | DEKKSKPKRINCFEKEKNLKDQYDKDAINASANKDGAQKW |
| 97 | GCFECIFFPMYKIESGDPNKRIIINKTRFKLFDFYLNLKG |
| | CKSCLRSTYHPYRSNVYIESNYDKLKREIGNFLQQKNIFQ |
| | RMRKAKVSEGKYLTNLDEYRLSCVAMHFKNRWLFFDSIQK |
| | VLRETIKQRLKQMRESYDEQAKTKRSKGHGRAKYEDQVRM |
| | IRRRAYSAQAHKLLDNGYITLFDYDDKEINKVCLTAINQE |
| | GFDIGGYLNSDIDNVMPPIEISFHLKWKYNEPILNIESPF |
| | SKAKISDYLRKIREDLNLERGKEGKARSKKNVRRKVLASK |
| | GEDGYKKIFTDFFSKWKEELEGNAMERVLSQSSGDIQWSK |
| | KKRIHYTTLVLNINLLDKKGVGNLKYYEIAEKTKILSFDK |
| | NENKFWPITIQVLLDGYEIGTEYDEIKQLNEKTSKQFTIY |
| | DPNTKIIKIPFTDSKAVPLGMLGINIATLKTVKKTERDIK |
| | VSKIFKGGLNSKIVSKIGKGIYAGYFPTVDKEILEEVEED |
| | TLDNEFSSKSQRNIFLKSIIKNYDKMLKEQLFDFYSFLVR |
| | NDLGVRFLTDRELQNIEDESFNLEKRFFETDRDRIARWFD |
| | NTNTDDGKEKFKKLANEIVDSYKPRLIRLPVVRVIKRIQP |
| | VKQREM |
| SEQ | KYSTRDFSELNEIQVTACKQDEFFKVIQNAWREIIKKRFL |
| ID | ENRENFIEKKIFKNKKGRGKRQESDKTIQRNRASVMKNFQ |
| NO: | LIENEKIILRAPSGHVACVFPVKVGLDIGGFKTDDLEKNI |
| 98 | FPPRTITINVFWKNRDRQRKGRKLEVWGIKARTKLIEKVH |
| | KWDKLEEVKKKRLKSLEQKQEKSLDNWSEVNNDSFYKVQI |
| | DELQEKIDKSLKGRTMNKILDNKAKESKEAEGLYIEWEKD |
| | FEGEMLRRIEASTGGEEKWGKRRQRRHTSLLLDIKNNSRG |
| | SKEIINFYSYAKQGKKEKKIEFFPFPLTITLDAEEESPLN |
| | IKSIPIEDKNATSKYFSIPFTETRATPLSILGDRVQKFKT |
| | KNISGAIKRNLGSSISSCKIVQNAETSAKSILSLPNVKED |
| | NNMEIFINTMSKNYFRAMMKQMESFIFEMEPKTLIDPYKE |
| | KAIKWFEVAASSRAKRKLKKLSKADIKKSELLLSNTEEFE |
| | KEKQEKLEALEKEIEEFYLPRIVRLQLTKTILETPVM |
| SEQ | KKLQLLGHKILLKEYDPNAVNAAANFETSTAELCGQCKMK |
| ID | PFKNKRRFQYTFGKNYHGCLSCIQNVYYAKKRIVQIAKEE |
| NO: | LKHQLTDSIASIPYKYTSLFSNTNSIDELYILKQERAAFF |
| 99 | SNTNSIDELYITGIENNIAFKVISAIWDEIIKKRRQRYAE |
| | SLTDTGTVKANRGHGGTAYKSNTRQEKIRALQKQTLHMVT |
| | NPYISLARYKNNYIVATLPRTIGMHIGAIKDRDPQKKLSD |
| | YAINFNVFWSDDRQLIELSTVQYTGDMVRKIEAETGENNK |
| | WGENMKRTKTSLLLEILTKKTTDELTFKDWAFSTKKEIDS |
| | VTKKTYQGFPIGIIFEGNESSVKFGSQNYFPLPFDAKITP |
| | PTAEGFRLDWLRKGSFSSQMKTSYGLAIYSNKVTNAIPAY |
| | VIKNMFYKIARAENGKQIKAKFLKKYLDIAGNNYVPFIIM |
| | QHYRVLDTFEEMPISQPKVIRLSLTKTQHIIIKKDKTDSK |
| | M |
| SEQ | NTSNLINLGKKAINISANYDANLEVGCKNCKFLSSNGNFP |
| ID | RQTNVKEGCHSCEKSTYEPSIYLVKIGERKAKYDVLDSLK |
| NO: | KFTFQSLKYQSKKSMKSRNKKPKELKEFVIFANKNKAFDV |
| 100 | IQKSYNHLILQIKKEINRMNSKKRKKNHKRRLFRDREKQL |
| | NKLRLIESSNLFLPRENKGNNHVFTYVAIHSVGRDIGVIG |
| | SYDEKLNFETELTYQLYFNDDKRLLYAYKPKQNKIIKIKE |
| | KLWNLRKEKEPLDLEYEKPLNKSITFSIKNDNLFKVSKDL |
| | MLRRAKFNIQGKEKLSKEERKINRDLIKIKGLVNSMSYGR |
| | FDELKKEKNIWSPHIYREVRQKEIKPCLIKNGDRIEIFEQ |
| | LKKKMERLRRFREKRQKKISKDLIFAERIAYNFHTKSIKN |
| | TSNKINIDQEAKRGKASYMRKRIGYETFKNKYCEQCLSKG |
| | NVYRNVQKGCSCFENPFDWIKKGDENLLPKKNEDLRVKGA |
| | FRDEALEKQIVKIAFNIAKGYEDFYDNLGESTEKDLKLKF |
| | KVGTTINEQESLKL |
| SEQ | TSNPIKLGKKAINISANYDSNLQIGCKNCKFLSYNGNFPR |
| ID | QTNVKEGCHSCEKSTYEPPVYTVRIGERRSKYDVLDSLKK |
| NO: | FIFLSLKYRQSKKMKTRSKGIRGLEEFVISANLKKAMDVI |
| 101 | QKSYRHLILNIKNEIVRMNGKKRNKNHKRLLFRDREKQLN |
| | KLRLIEGSSFFKPPTVKGDNSIFTCVAIHNIGRDIGIAGD |
| | YFDKLEPKIELTYQLYYEYNPKKESEINKRLLYAYKPKQN |
| | KIIEIKEKLWNLRKEKSPLDLEYEKPLTKSITFLVKRDGV |
| | FRISKDLMLRKAKFIIQGKEKLSKEERKINRDLIKIKSNI |
| | ISLTYGRFDELKKDKTIWSPHIFRDVKQGKITPCIERKGD |
| | RMDIFQQLRKKSERLRENRKKRQKKISKDLIFAERIAYNF |
| | HTKSIKNTSNLINIKHEAKRGKASYMRKRIGNETFRIKYC |
| | EQCFPKNNVYKNVQKGCSCFEDPFEYIKKGNEDLIPNKNQ |
| | DLKAKGAFRDDALEKQIIKVAFNIAKGYEDFYENLKKTTE |
| | KDIRLKFKVGTIISEEM |
| SEQ | NNSINLSKKAINISANYDANLQVRCKNCKFLSSNGNFPRQ |
| ID | TDVKEGCHSCEKSTYEPPVYDVKIGEIKAKYEVLDSLKKF |
| NO: | TFQSLKYQLSKSMKFRSKKIKELKEFVIFAKESKALNVIN |
| 102 | RSYKHLILNIKNDINRMNSKKRIKNHKGRLFLDRQKQLSK |
| | LKLIEGSSFFVPAKNVGNKSVFTCVAIHSIGRDIGIAGLY |
| | DSFTKPVNEITYQIFFSGERRLLYAYKPKQLKILSIKENL |
| | WSLKNEKKPLDLLYEKPLGKNLNFNVKGGDLFRVSKDLMI |
| | RNAKFNVHGRQRLSDEERLINRNFIKIKGEVVSLSYGRFE |
| | ELKKDRKLWSPHIFKDVRQNKIKPCLVMQGQRIDIFEQLK |
| | RKLELLKKIRKSRQKKLSKDLIFGERIAYNFHTKSIKNTS |
| | NKINIDSDAKRGRASYMRKRIGNETFKLKYCDVCFPKANV |
| | YRRVQNGCSCSENPYNYIKKGDKDLLPKKDEGLAIKGAFR |
| | DEKLNKQIIKVAFNIAKGYEDFYDDLKKRTEKDVDLKFKI |
| | GTTVLDQKPMEIFDGIVITWL |
| SEQ | LLTTVVETNNLAKKAINVAANFDANIDRQYYRCTPNLCRF |
| ID | IAQSPRETKEKDAGCSSCTQSTYDPKVYVIKIGKLLAKYE |
| NO: | ILKSLKRFLFMNRYFKQKKTERAQQKQKIGTELNEMSIFA |
| 103 | KATNAMEVIKRATKHCTYDIIPETKSLQMLKRRRHRVKVR |
| | SLLKILKERRMKIKKIPNTFIEIPKQAKKNKSDYYVAAAL |
| | KSCGIDVGLCGAYEKNAEVEAEYTYQLYYEYKGNSSTKRI |
| | LYCYNNPQKNIREFWEAFYIQGSKSHVNTPGTIRLKMEKF |
| | LSPITIESEALDFRVWNSDLKIRNGQYGFIKKRSLGKEAR |
| | EIKKGMGDIKRKIGNLTYGKSPSELKSIHVYRTERENPKK |
| | PRAARKKEDNFMEIFEMQRKKDYEVNKKRRKEATDAAKIM |
| | DFAEEPIRHYHTNNLKAVRRIDMNEQVERKKTSVFLKRIM |
| | QNGYRGNYCRKCIKAPEGSNRDENVLEKNEGCLDCIGSEF |
| | IWKKSSKEKKGLWHTNRLLRRIRLQCFTTAKAYENFYNDL |
| | FEKKESSLDIIKLKVSITTKSM |
| SEQ | ASTMNLAKQAINFAANYDSNLEIGCKGCKFMSTWSKKSNP |
| ID | KFYPRQNNQANKCHSCTYSTGEPEVPIIEIGERAAKYKIF |
| NO: | TALKKFVFMSVAYKERRRQRFKSKKPKELKELAICSNREK |
| 104 | AMEVIQKSVVHCYGDVKQEIPRIRKIKVLKNHKGRLFYKQ |
| | KRSKIKIAKLEKGSFFKTFIPKVHNNGCHSCHEASLNKPI |
| | LVTTALNTIGADIGLINDYSTIAPTETDISWQVYYEFIPN |
| | GDSEAVKKRLLYFYKPKGALIKSIRDKYFKKGHENAVNTG |
| | FFKYQGKIVKGPIKFVNNELDFARKPDLKSMKIKRAGFAI |
| | PSAKRLSKEDREINRESIKIKNKIYSLSYGRKKTLSDKDI |
| | IKHLYRPVRQKGVKPLEYRKAPDGFLEFFYSLKRKERRLR |
| | KQKEKRQKDMSEIIDAADEFAWHRHTGSIKKTTNHINFKS |
| | EVKRGKVPIMKKRIANDSFNTRHCGKCVKQGNAINKYYIE |
| | KQKNCFDCNSIEFKWEKAALEKKGAFKLNKRLQYIVKACF |
| | NVAKAYESFYEDFRKGEEESLDLKFKIGTTTTLKQYPQNK |
| | ARAM |
| SEQ | HSHNLMLTKLGKQAINFAANYDANLEIGCKNCKFLSYSPK |
| ID | QANPKKYPRQTDVHEDGNIACHSCMQSTKEPPVYIVPIGE |
| NO: | RKSKYEILTSLNKFTFLALKYKEKKRQAFRAKKPKELQEL |
| 105 | AIAFNKEKAIKVIDKSIQHLILNIKPEIARIQRQKRLKNR |
| | KGKLLYLHKRYAIKMGLIKNGKYFKVGSPKKDGKKLLVLC |
| | ALNTIGRDIGIIGNIEENNRSETEITYQLYFDCLDANPNE |
| | LRIKEIEYNRLKSYERKIKRLVYAYKPKQTKILEIRSKFF |
| | SKGHENKVNTGSFNFENPLNKSISIKVKNSAFDFKIGAPF |
| | IMLRNGKFHIPTKKRLSKEEREINRTLSKIKGRVFRLTYG |
| | RNISEQGSKSLHIYRKERQHPKLSLEIRKQPDSFIDEFEK |
| | LRLKQNFISKLKKQRQKKLADLLQFADRIAYNYHTSSLEK |
| | TSNFINYKPEVKRGRTSYIKKRIGNEGFEKLYCETCIKSN |
| | DKENAYAVEKEELCFVCKAKPFTWKKTNKDKLGIFKYPSR |
| | IKDFIRAAFTVAKSYNDFYENLKKKDLKNEIFLKFKIGLI |
| | LSHEKKNHISIAKSVAEDERISGKSIKNILNKSIKLEKNC |
| | YSCFFHKEDM |
| SEQ | SLERVIDKRNLAKKAINIAANFDANINKGFYRCETNQCMF |
| ID | IAQKPRKTNNTGCSSCLQSTYDPVIYVVKVGEMLAKYEIL |
| NO: | KSLKRFVFMNRSFKQKKTEKAKQKERIGGELNEMSIFANA |
| 106 | ALAMGVIKRAIRHCHVDIRPEINRLSELKKTKHRVAAKSL |
| | VKIVKQRKTKWKGIPNSFIQIPQKARNKDADFYVASALKS |
| | GGIDIGLCGTYDKKPHADPRWTYQLYFDTEDESEKRLLYC |
| | YNDPQAKIRDFWKTFYERGNPSMVNSPGTIEFRMEGFFEK |
| | MTPISIESKDFDFRVWNKDLLIRRGLYEIKKRKNLNRKAR |
| | EIKKAMGSVKRVLANMTYGKSPTDKKSIPVYRVEREKPKK |
| | PRAVRKEENELADKLENYRREDFLIRNRRKREATEIAKII |
| | DAAEPPIRHYHTNHLRAVKRIDLSKPVARKNTSVFLKRIM |
| | QNGYRGNYCKKCIKGNIDPNKDECRLEDIKKCICCEGTQN |
| | IWAKKEKLYTGRINVLNKRIKQMKLECFNVAKAYENFYDN |
| | LAALKEGDLKVLKLKVSIPALNPEASDPEEDM |
| SEQ | NASINLGKRAINLSANYDSNLVIGCKNCKFLSFNGNFPRQ |
| ID | TNVREGCHSCDKSTYAPEVYIVKIGERKAKYDVLDSLKKF |
| NO: | TFQSLKYQIKKSMRERSKKPKELLEFVIFANKDKAFNVIQ |
| 107 | KSYEHLILNIKQEINRMNGKKRIKNHKKRLFKDREKQLNK |
| | LRLIGSSSLFFPRENKGDKDLFTYVAIHSVGRDIGVAGSY |
| | ESHIEPISDLTYQLFINNEKRLLYAYKPKQNKIIELKENL |
| | WNLKKEKKPLDLEFTKPLEKSITFSVKNDKLFKVSKDLML |
| | RQAKFNIQGKEKLSKEERQINRDFSKIKSNVISLSYGRFE |
| | ELKKEKNIWSPHIYREVKQKEIKPCIVRKGDRIELFEQLK |
| | RKMDKLKKFRKERQKKISKDLNFAERIAYNFHTKSIKNTS |
| | NKINIDQEAKRGKASYMRKRIGNESFRKKYCEQCFSVGNV |
| | YHNVQNGCSCFDNPIELIKKGDEGLIPKGKEDRKYKGALR |
| | DDNLQMQIIRVAFNIAKGYEDFYNNLKEKTEKDLKLKFKI |
| | GTTISTQESNNKEM |
| SEQ | SNLIKLGKQAINFAANYDANLEVGCKNCKFLSSTNKYPRQ |
| ID | TNVHLDNKMACRSCNQSTMEPAIYIVRIGEKKAKYDIYNS |
| NO: | LTKFNFQSLKYKAKRSQRFKPKQPKELQELSIAVRKEKAL |
| 108 | DIIQKSIDHLIQDIRPEIPRIKQQKRYKNHVGKLFYLQKR |
| | RKNKLNLIGKGSFFKVFSPKEKKNELLVICALTNIGRDIG |
| | LIGNYNTIINPLFEVTYQLYYDYIPKKNNKNVQRRLLYAY |
| | KSKNEKILKLKEAFFKRGHENAVNLGSFSYEKPLEKSLTL |
| | KIKNDKDDFQVSPSLRIRTGRFFVPSKRNLSRQEREINRR |
| | LVKIKSKIKNMTYGKFETARDKQSVHIFRLERQKEKLPLQ |
| | FRKDEKEFMEEFQKLKRRTNSLKKLRKSRQKKLADLLQLS |
| | EKVVYNNHTGTLKKTSNFLNFSSSVKRGKTAYIKELLGQE |
| | GFETLYCSNCINKGQKTRYNIETKEKCFSCKDVPFVWKKK |
| | STDKDRKGAFLFPAKLKDVIKATFTVAKAYEDFYDNLKSI |
| | DEKKPYIKFKIGLILAHVRHEHKARAKEEAGQKNIYNKPI |
| | KIDKNCKECFFFKEEAM |
| SEQ | NTTRKKFRKRTGFPQSDNIKLAYCSAIVRAANLDADIQKK |
| ID | HNQCNPNLCVGIKSNEQSRKYEHSDRQALLCYACNQSTGA |
| NO: | PKVDYIQIGEIGAKYKILQMVNAYDFLSLAYNLTKLRNGK |
| 109 | SRGHQRMSQLDEVVIVADYEKATEVIKRSINHLLDDIRGQ |
| | LSKLKKRTQNEHITEHKQSKIRRKLRKLSRLLKRRRWKWG |
| | TIPNPYLKNWVFTKKDPELVTVALLHKLGRDIGLVNRSKR |
| | RSKQKLLPKVGFQLYYKWESPSLNNIKKSKAKKLPKRLLI |
| | PYKNVKLFDNKQKLENAIKSLLESYQKTIKVEFDQFFQNR |
| | TEEIIAEEQQTLERGLLKQLEKKKNEFASQKKALKEEKKK |
| | IKEPRKAKLLMEESRSLGFLMANVSYALFNTTIEDLYKKS |
| | NVVSGCIPQEPVVVFPADIQNKGSLAKILFAPKDGFRIKF |
| | SGQHLTIRTAKFKIRGKEIKILTKTKREILKNIEKLRRVW |
| | YREQHYKLKLFGKEVSAKPRFLDKRKTSIERRDPNKLADQ |
| | TDDRQAELRNKEYELRHKQHKMAERLDNIDTNAQNLQTLS |
| | FWVGEADKPPKLDEKDARGFGVRTCISAWKWFMEDLLKKQ |
| | EEDPLLKLKLSIM |
| SEQ | PKKPKFQKRTGFPQPDNLRKEYCLAIVRAANLDADFEKKC |
| ID | TKCEGIKTNKKGNIVKGRTYNSADKDNLLCYACNISTGAP |
| NO: | AVDYVFVGALEAKYKILQMVKAYDFHSLAYNLAKLWKGRG |
| 110 | RGHQRMGGLNEVVIVSNNEKALDVIEKSLNHFHDEIRGEL |
| | SRLKAKFQNEHLHVHKESKLRRKLRKISRLLKRRRWKWDV |
| | IPNSYLRNFTFTKTRPDFISVALLHRVGRDIGLVTKTKIP |
| | KPTDLLPQFGFQIYYTWDEPKLNKLKKSRLRSEPKRLLVP |
| | YKKIELYKNKSVLEEAIRHLAEVYTEDLTICFKDFFETQK |
| | RKFVSKEKESLKRELLKELTKLKKDFSERKTALKRDRKEI |
| | KEPKKAKLLMEESRSLGFLAANTSYALFNLIAADLYTKSK |
| | KACSTKLPRQLSTILPLEIKEHKSTTSLAIKPEEGFKIRF |
| | SNTHLSIRTPKFKMKGADIKALTKRKREILKNATKLEKSW |
| | YGLKHYKLKLYGKEVAAKPRFLDKRNPSIDRRDPKELMEQ |
| | IENRRNEVKDLEYEIRKGQHQMAKRLDNVDTNAQNLQTKS |
| | FWVGEADKPPELDSMEAKKLGLRTCISAWKWFMKDLVLLQ |
| | EKSPNLKLKLSLTEM |
| SEQ | KFSKRQEGFLIPDNIDLYKCLAIVRSANLDADVQGHKSCY |
| ID | GVKKNGTYRVKQNGKKGVKEKGRKYVFDLIAFKGNIEKIP |
| NO: | HEAIEEKDQGRVIVLGKFNYKLILNIEKNHNDRASLEIKN |
| 111 | KIKKLVQISSLETGEFLSDLLSGKIGIDEVYGIIEPDVFS |
| | GKELVCKACQQSTYAPLVEYMPVGELDAKYKILSAIKGYD |
| | FLSLAYNLSRNRANKKRGHQKLGGGELSEVVISANYDKAL |
| | NVIKRSINHYHVEIKPEISKLKKKMQNEPLKVMKQARIRR |
| | ELHQLSRKVKRLKWKWGMIPNPELQNIIFEKKEKDFVSYA |
| | LLHTLGRDIGLFKDTSMLQVPNISDYGFQIYYSWEDPKLN |
| | SIKKIKDLPKRLLIPYKRLDFYIDTILVAKVIKNLIELYR |
| | KSYVYETFGEEYGYAKKAEDILFDWDSINLSEGIEQKIQK |
| | IKDEFSDLLYEARESKRQNFVESFENILGLYDKNFASDRN |
| | SYQEKIQSMIIKKQQENIEQKLKREFKEVIERGFEGMDQN |
| | KKYYKVLSPNIKGGLLYTDTNNLGFFRSHLAFMLLSKISD |
| | DLYRKNNLVSKGGNKGILDQTPETMLTLEFGKSNLPNISI |
| | KRKFFNIKYNSSWIGIRKPKFSIKGAVIREITKKVRDEQR |
| | LIKSLEGVWHKSTHFKRWGKPRFNLPRHPDREKNNDDNLM |
| | ESITSRREQIQLLLREKQKQQEKMAGRLDKIDKEIQNLQT |
| | ANFQIKQIDKKPALTEKSEGKQSVRNALSAWKWFMEDLIK |
| | YQKRTPILQLKLAKM |
| SEQ | KFSKRQEGFVIPENIGLYKCLAIVRSANLDADVQGHVSCY |
| ID | GVKKNGTYVLKQNGKKSIREKGRKYASDLVAFKGDIEKIP |
| NO: | FEVIEEKKKEQSIVLGKFNYKLVLDVMKGEKDRASLTMKN |
| 112 | KSKKLVQVSSLGTDEFLLTLLNEKFGIEEIYGIIEPEVFS |
| | GKKLVCKACQQSTYAPLVEYMPVGELDSKYKILSAIKGYD |
| | FLSLAYNLARHRSNKKRGHQKLGGGELSEVVISANNAKAL |
| | NVIKRSLNHYYSEIKPEISKLRKKMQNEPLKVGKQARMRR |
| | ELHQLSRKVKRLKWKWGKIPNLELQNITFKESDRDFISYA |
| | LLHTLGRDIGMFNKTEIKMPSNILGYGFQIYYDWEEPKLN |
| | TIKKSKNTPKRILIPYKKLDFYNDSILVARAIKELVGLFQ |
| | ESYEWEIFGNEYNYAKEAEVELIKLDEESINGNVEKKLQR |
| | IKENFSNLLEKAREKKRQNFIESFESIARLYDESFTADRN |
| | EYQREIQSFIIEKQKQSIEKKLKNEFKKIVEKKFNEQEQG |
| | KKHYRVLNPTIINEFLPKDKNNLGFLRSKIAFILLSKISD |
| | DLYKKSNAVSKGGEKGIIKQQPETILDLEFSKSKLPSINI |
| | KKKLFNIKYTSSWLGIRKPKFNIKGAKIREITRRVRDVQR |
| | TLKSAESSWYASTHFRRWGFPRFNQPRHPDKEKKSDDRLI |
| | ESITLLREQIQILLREKQKGQKEMAGRLDDVDKKIQNLQT |
| | ANFQIKQTGDKPALTEKSAGKQSFRNALSAWKWFMENLLK |
| | YQNKTPDLKLKIARTVM |
| SEQ | KWIEPNNIDFNKCLAITRSANLDADVQGHKMCYGIKTNGT |
| ID | YKAIGKINKKHNTGIIEKRRTYVYDLIVTKEKNEKIVKKT |
| NO: | DFMAIDEEIEFDEKKEKLLKKYIKAEVLGTGELIRKDLND |
| 113 | GEKFDDLCSIEEPQAFRRSELVCKACNQSTYASDIRYIPI |
| | GEIEAKYKILKAIKGYDFLSLKYNLGRLRDSKKRGHQKMG |
| | QGELKEFVICANKEKALDVIKRSLNHYLNEVKDEISRLNK |
| | KMQNEPLKVNDQARWRRELNQISRRLKRLKWKWGEIPNPE |
| | LKNLIFKSSRPEFVSYALIHTLGRDIGLINETELKPNNIQ |
| | EYGFQIYYKWEDPELNHIKKVKNIPKRFIIPYKNLDLFGK |
| | YTILSRAIEGILKLYSSSFQYKSFKDPNLFAKEGEKKITN |
| | EDFELGYDEKIKKIKDDFKSYKKALLEKKKNTLEDSLNSI |
| | LSVYEQSLLTEQINNVKKWKEGLLKSKESIHKQKKIENIE |
| | DIISRIEELKNVEGWIRTKERDIVNKEETNLKREIKKELK |
| | DSYYEEVRKDFSDLKKGEESEKKPFREEPKPIVIKDYIKF |
| | DVLPGENSALGFFLSHLSFNLFDSIQYELFEKSRLSSSKH |
| | PQIPETILDL |
| SEQ | FRKFVKRSGAPQPDNLNKYKCIAIVRAANLDADIMSNESS |
| ID | NCVMCKGIKMNKRKTAKGAAKTTELGRVYAGQSGNLLCTA |
| NO: | CTKSTMGPLVDYVPIGRIRAKYTILRAVKEYDFLSLAYNL |
| 114 | ARTRVSKKGGRQKMHSLSELVIAAEYEIAWNIIKSSVIHY |
| | HQETKEEISGLRKKLQAEHIHKNKEARIRREMHQISRRIK |
| | RLKWKWHMIPNSELHNFLFKQQDPSFVAVALLHTLGRDIG |
| | MINKPKGSAKREFIPEYGFQIYYKWMNPKLNDINKQKYRK |
| | MPKRSLIPYKNLNVFGDRELIENAMHKLLKLYDENLEVKG |
| | SKFFKTRVVAISSKESEKLKRDLLWKGELAKIKKDFNADK |
| | NKMQELFKEVKEPKKANALMKQSRNMGFLLQNISYGALGL |
| | LANRMYEASAKQSKGDATKQPSIVIPLEMEFGNAFPKLLL |
| | RSGKFAMNVSSPWLTIRKPKFVIKGNKIKNITKLMKDEKA |
| | KLKRLETSYHRATHFRPTLRGSIDWDSPYFSSPKQPNTHR |
| | RSPDRLSADITEYRGRLKSVEAELREGQRAMAKKLDSVDM |
| | TASNLQTSNFQLEKGEDPRLTEIDEKGRSIRNCISSWKKF |
| | MEDLMKAQEANPVIKIKIALKDESSVLSEDSM |
| SEQ | KFHPENLNKSYCLAIVRAANLDADIQGHINCIGIKSNKSD |
| ID | RNYENKLESLQNVELLCKACTKSTYKPNINSVPVGEKKAK |
| NO: | YSILSEIKKYDFNSLVYNLKKYRKGKSRGHQKLNELRELV |
| 115 | ITSEYKKALDVINKSVNHYLVNIKNKMSKLKKILQNEHIH |
| | VGTLARIRRERNRISRKLDHYRKKWKFVPNKILKNYVFKN |
| | QSPDFVSVALLHKLGRDIGLITKTAILQKSFPEYSLQLYY |
| | KYDTPKLNYLKKSKFKSLPKRILISYKYPKFDINSNYIEE |
| | SIDKLLKLYEESPIYKNNSKIIEFFKKSEDNLIKSENDSL |
| | KRGIMKEFEKVTKNFSSKKKKLKEELKLKNEDKNSKMLAK |
| | VSRPIGFLKAYLSYMLFNIISNRIFEFSRKSSGRIPQLPS |
| | CIINLGNQFENFKNELQDSNIGSKKNYKYFCNLLLKSSGF |
| | NISYEEEHLSIKTPNFFINGRKLKEITSEKKKIRKENEQL |
| | IKQWKKLTFFKPSNLNGKKTSDKIRFKSPNNPDIERKSED |
| | NIVENIAKVKYKLEDLLSEQRKEFNKLAKKHDGVDVEAQC |
| | LQTKSFWIDSNSPIKKSLEKKNEKVSVKKKMKAIRSCISA |
| | WKWFMADLIEAQKETPMIKLKLALM |
| SEQ | TTLVPSHLAGIEVMDETTSRNEDMIQKETSRSNEDENYLG |
| ID | VKNKCGINVHKSGRGSSKHEPNMPPEKSGEGQMPKQDSTE |
| NO: | MQQRFDESVTGETQVSAGATASIKTDARANSGPRVGTARA |
| 116 | LIVKASNLDRDIKLGCKPCEYIRSELPMGKKNGCNHCEKS |
| | SDIASVPKVESGFRKAKYELVRRFESFAADSISRHLGKEQ |
| | ARTRGKRGKKDKKEQMGKVNLDEIAILKNESLIEYTENQI |
| | LDARSNRIKEWLRSLRLRLRTRNKGLKKSKSIRRQLITLR |
| | RDYRKWIKPNPYRPDEDPNENSLRLHTKLGVDIGVQGGDN |
| | KRMNSDDYETSFSITWRDTATRKICFTKPKGLLPRHMKFK |
| | LRGYPELILYNEELRIQDSQKFPLVDWERIPIFKLRGVSL |
| | GKKKVKALNRITEAPRLVVAKRIQVNIESKKKKVLTRYVY |
| | NDKSINGRLVKAEDSNKDPLLEFKKQAEEINSDAKYYENQ |
| | EIAKNYLWGCEGLHKNLLEEQTKNPYLAFKYGFLNIV |
| SEQ | LDFKRTCSQELVLLPEIEGLKLSGTQGVTSLAKKLINKAA |
| ID | NVDRDESYGCHHCIHTRTSLSKPVKKDCNSCNQSTNHPAV |
| NO: | PITLKGYKIAFYELWHRFTSWAVDSISKALHRNKVMGKVN |
| 117 | LDEYAVVDNSHIVCYAVRKCYEKRQRSVRLHKRAYRCRAK |
| | HYNKSQPKVGRIYKKSKRRNARNLKKEAKRYFQPNEITNG |
| | SSDALFYKIGVDLGIAKGTPETEVKVDVSICFQVYYGDAR |
| | RVLRVRKMDELQSFHLDYTGKLKLKGIGNKDTFTIAKRNE |
| | SLKWGSTKYEVSRAHKKFKPFGKKGSVKRKCNDYFRSIAS |
| | WSCEAASQRAQSNLKNAFPYQKALVKCYKNLDYKGVKKND |
| | MWYRLCSNRIFRYSRIAEDIAQYQSDKGKAKFEFVILAQS |
| | VAEYDISAIM |
| SEQ | VFLTDDKRKTALRKIRSAFRKTAEIALVRAQEADSLDRQA |
| ID | KKLTIETVSFGAPGAKNAFIGSLQGYNWNSHRANVPSSGS |
| NO: | AKDVFRITELGLGIPQSAHEASIGKSFELVGNVVRYTANL |
| 118 | LSKGYKKGAVNKGAKQQREIKGKEQLSFDLISNGPISGDK |
| | LINGQKDALAWWLIDKMGFHIGLAMEPLSSPNTYGITLQA |
| | FWKRHTAPRRYSRGVIRQWQLPFGRQLAPLIHNFFRKKGA |
| | SIPIVLTNASKKLAGKGVLLEQTALVDPKKWWQVKEQVTG |
| | PLSNIWERSVPLVLYTATFTHKHGAAHKRPLTLKVIRISS |
| | GSVFLLPLSKVTPGKLVRAWMPDINILRDGRPDEAAYKGP |
| | DLIRARERSFPLAYTCVTQIADEWQKRALESNRDSITPLE |
| | AKLVTGSDLLQIHSTVQQAVEQGIGGRISSPIQELLAKDA |
| | LQLVLQQLFMTVDLLRIQWQLKQEVADGNTSEKAVGWAIR |
| | ISNIHKDAYKTAIEPCTSALKQAWNPLSGFEERTFQLDAS |
| | IVRKRSTAKTPDDELVIVLRQQAAEMTVAVTQSVSKELME |
| | LAVRHSATLHLLVGEVASKQLSRSADKDRGAMDHWKLLSQ |
| | SM |
| SEQ | EDLLQKALNTATNVAAIERHSCISCLFTESEIDVKYKTPD |
| ID | KIGQNTAGCQSCTFRVGYSGNSHTLPMGNRIALDKLRETI |
| NO: | QRYAWHSLLFNVPPAPTSKRVRAISELRVAAGRERLFTVI |
| 119 | TFVQTNILSKLQKRYAANWTPKSQERLSRLREEGQHILSL |
| | LESGSWQQKEVVREDQDLIVCSALTKPGLSIGAFCRPKYL |
| | KPAKHALVLRLIFVEQWPGQIWGQSKRTRRMRRRKDVERV |
| | YDISVQAWALKGKETRISECIDTMRRHQQAYIGVLPFLIL |
| | SGSTVRGKGDCPILKEITRMRYCPNNEGLIPLGIFYRGSA |
| | NKLLRVVKGSSFTLPMWQNIETLPHPEPFSPEGWTATGAL |
| | YEKNLAYWSALNEAVDWYTGQILSSGLQYPNQNEFLARLQ |
| | NVIDSIPRKWFRPQGLKNLKPNGQEDIVPNEFVIPQNAIR |
| | AHHVIEWYHKTNDLVAKTLLGWGSQTTLNQTRPQGDLRFT |
| | YTRYYFREKEVPEV |
| SEQ | VPKKKLMRELAKKAVFEAIFNDPIPGSFGCKRCTLIDGAR |
| ID | VTDAIEKKQGAKRCAGCEPCTFHTLYDSVKHALPAATGCD |
| NO: | RTAIDTGLWEILTALRSYNWMSFRRNAVSDASQKQVWSIE |
| 120 | ELAIWADKERALRVILSALTHTIGKLKNGFSRDGVWKGGK |
| | QLYENLAQKDLAKGLFANGEIFGKELVEADHDMLAWTIVP |
| | NHQFHIGLIRGNWKPAAVEASTAFDARWLTNGAPLRDTRT |
| | HGHRGRRFNRTEKLTVLCIKRDGGVSEEFRQERDYELSVM |
| | LLQPKNKLKPEPKGELNSFEDLHDHWWFLKGDEATALVGL |
| | TSDPTVGDFIQLGLYIRNPIKAHGETKRRLLICFEPPIKL |
| | PLRRAFPSEAFKTWEPTINVFRNGRRDTEAYYDIDRARVF |
| | EFPETRVSLEHLSKQWEVLRLEPDRENTDPYEAQQNEGAE |
| | LQVYSLLQEAAQKMAPKVVIDPFGQFPLELFSTFVAQLFN |
| | APLSDTKAKIGKPLDSGFVVESHLHLLEEDFAYRDFVRVT |
| | FMGTEPTFRVIHYSNGEGYWKKTVLKGKNNIRTALIPEGA |
| | KAAVDAYKNKRCPLTLEAAILNEEKDRRLVLGNKALSLLA |
| | QTARGNLTILEALAAEVLRPLSGTEGVVHLHACVTRHSTL |
| | TESTETDNM |
| SEQ | VEKLFSERLKRAMWLKNEAGRAPPAETLTLKHKRVSGGHE |
| ID | KVKEELQRVLRSLSGTNQAAWNLGLSGGREPKSSDALKGE |
| NO: | KSRVVLETVVFHSGHNRVLYDVIEREDQVHQRSSIMHMRR |
| 121 | KGSNLLRLWGRSGKVRRKMREEVAEIKPVWHKDSRWLAIV |
| | EEGRQSVVGISSAGLAVFAVQESQCTTAEPKPLEYVVSIW |
| | FRGSKALNPQDRYLEFKKLKTTEALRGQQYDPIPFSLKRG |
| | AGCSLAIRGEGIKFGSRGPIKQFFGSDRSRPSHADYDGKR |
| | RLSLFSKYAGDLADLTEEQWNRTVSAFAEDEVRRATLANI |
| | QDFLSISHEKYAERLKKRIESIEEPVSASKLEAYLSAIFE |
| | TFVQQREALASNFLMRLVESVALLISLEEKSPRVEFRVAR |
| | YLAESKEGFNRKAM |
| SEQ | VVITQSELYKERLLRVMEIKNDRGRKEPRESQGLVLRFTQ |
| ID | VTGGQEKVKQKLWLIFEGFSGTNQASWNFGQPAGGRKPNS |
| NO: | GDALKGPKSRVTYETVVFHFGLRLLSAVIERHNLKQQRQT |
| 122 | MAYMKRRAAARKKWARSGKKCSRMRNEVEKIKPKWHKDPR |
| | WFDIVKEGEPSIVGISSAGFAIYIVEEPNFPRQDPLEIEY |
| | AISIWFRRDRSQYLTFKKIQKAEKLKELQYNPIPFRLKQE |
| | KTSLVFESGDIKFGSRGSIEHFRDEARGKPPKADMDNNRR |
| | LTMFSVFSGNLTNLTEEQYARPVSGLLAPDEKRMPTLLKK |
| | LQDFFTPIHEKYGERIKQRLANSEASKRPFKKLEEYLPAI |
| | YLEFRARREGLASNWVLVLINSVRTLVRIKSEDPYIEFKV |
| | SQYLLEKEDNKAL |
| SEQ | KQDALFEERLKKAIFIKRQADPLQREELSLLPPNRKIVTG |
| ID | GHESAKDTLKQILRAINGTNQASWNPGTPSGKRDSKSADA |
| NO: | LAGPKSRVKLETVVFHVGHRLLKKVVEYQGHQKQQHGLKA |
| 123 | FMRTCAAMRKKWKRSGKVVGELREQLANIQPKWHYDSRPL |
| | NLCFEGKPSVVGLRSAGIALYTIQKSVVPVKEPKPIEYAV |
| | SIWFRGPKAMDREDRCLEFKKLKIATELRKLQFEPIVSTL |
| | TQGIKGFSLYIQGNSVKFGSRGPIKYFSNESVRQRPPKAD |
| | PDGNKRLALFSKFSGDLSDLTEEQWNRPILAFEGIIRRAT |
| | LGNIQDYLTVGHEQFAISLEQLLSEKESVLQMSIEQQRLK |
| | KNLGKKAENEWVESFGAEQARKKAQGIREYISGFFQEYCS |
| | QREQWAENWVQQLNKSVRLFLTIQDSTPFIEFRVARYLPK |
| | GEKKKGKAM |
| SEQ | ANHAERHKRLRKEANRAANRNRPLVADCDTGDPLVGICRL |
| ID | LRRGDKMQPNKTGCRSCEQVEPELRDAILVSGPGRLDNYK |
| NO: | YELFQRGRAMAVHRLLKRVPKLNRPKKAAGNDEKKAENKK |
| 124 | SEIQKEKQKQRRMMPAVSMKQVSVADFKHVIENTVRHLFG |
| | DRRDREIAECAALRAASKYFLKSRRVRPRKLPKLANPDHG |
| | KELKGLRLREKRAKLKKEKEKQAELARSNQKGAVLHVATL |
| | KKDAPPMPYEKTQGRNDYTTFVISAAIKVGATRGTKPLLT |
| | PQPREWQCSLYWRDGQRWIRGGLLGLQAGIVLGPKLNREL |
| | LEAVLQRPIECRMSGCGNPLQVRGAAVDFFMTTNPFYVSG |
| | AAYAQKKFKPFGTKRASEDGAAAKAREKLMTQLAKVLDKV |
| | VTQAAHSPLDGIWETRPEAKLRAMIMALEHEWIFLRPGPC |
| | HNAAEEVIKCDCTGGHAILWALIDEARGALEHKEFYAVTR |
| | AHTHDCEKQKLGGRLAGFLDLLIAQDVPLDDAPAARKIKT |
| | LLEATPPAPCYKAATSIATCDCEGKFDKLWAIIDATRAGH |
| | GTEDLWARTLAYPQNVNCKCKAGKDLTHRLADFLGLLIKR |
| | DGPFRERPPHKVTGDRKLVFSGDKKCKGHQYVILAKAHNE |
| | EVVRAWISRWGLKSRTNKAGYAATELNLLLNWLSICRRRW |
| | MDMLTVQRDTPYIRMKTGRLVVDDKKERKAM |
| SEQ | AKQREALRVALERGIVRASNRTYTLVTNCTKGGPLPEQCR |
| ID | MIERGKARAMKWEPKLVGCGSCAAATVDLPAIEEYAQPGR |
| NO: | LDVAKYKLTTQILAMATRRMMVRAAKLSRRKGQWPAKVQE |
| 125 | EKEEPPEPKKMLKAVEMRPVAIVDFNRVIQTTIEHLWAER |
| | ANADEAELKALKAAAAYFGPSLKIRARGPPKAAIGRELKK |
| | AHRKKAYAERKKARRKRAELARSQARGAAAHAAIRERDIP |
| | PMAYERTQGRNDVTTIPIAAAIKIAATRGARPLPAPKPMK |
| | WQCSLYWNEGQRWIRGGMLTAQAYAHAANIHRPMRCEMWG |
| | VGNPLKVRAFEGRVADPDGAKGRKAEFRLQTNAFYVSGAA |
| | YRNKKFKPFGTDRGGIGSARKKRERLMAQLAKILDKVVSQ |
| | AAHSPLDDIWHTRPAQKLRAMIKQLEHEWMFLRPQAPTVE |
| | GTKPDVDVAGNMQRQIKALMAPDLPPIEKGSPAKRFTGDK |
| | RKKGERAVRVAEAHSDEVVTAWISRWGIQTRRNEGSYAAQ |
| | ELELLLNWLQICRRRWLDMTAAQRVSPYIRMKSGRMITDA |
| | ADEGVAPIPLVENM |
| SEQ | KSISGRSIKHMACLKDMLKSEITEIEEKQKKESLRKWDYY |
| ID | SKFSDEILFRRNLNVSANHDANACYGCNPCAFLKEVYGFR |
| NO: | IERRNNERIISYRRGLAGCKSCVQSTGYPPIEFVRRKFGA |
| 126 | DKAMEIVREVLHRRNWGALARNIGREKEADPILGELNELL |
| | LVDARPYFGNKSAANETNLAFNVITRAAKKFRDEGMYDIH |
| | KQLDIHSEEGKVPKGRKSRLIRIERKHKAIHGLDPGETWR |
| | YPHCGKGEKYGVWLNRSRLIHIKGNEYRCLTAFGTTGRRM |
| | SLDVACSVLGHPLVKKKRKKGKKTVDGTELWQIKKATETL |
| | PEDPIDCTFYLYAAKPTKDPFILKVGSLKAPRWKKLHKDF |
| | FEYSDTEKTQGQEKGKRVVRRGKVPRILSLRPDAKFKVSI |
| | WDDPYNGKNKEGTLLRMELSGLDGAKKPLILKRYGEPNTK |
| | PKNFVFWRPHITPHPLTFTPKHDFGDPNKKTKRRRVFNRE |
| | YYGHLNDLAKMEPNAKFFEDREVSNKKNPKAKNIRIQAKE |
| | SLPNIVAKNGRWAAFDPNDSLWKLYLHWRGRRKTIKGGIS |
| | QEFQEFKERLDLYKKHEDESEWKEKEKLWENHEKEWKKTL |
| | EIHGSIAEVSQRCVMQSMMGPLDGLVQKKDYVHIGQSSLK |
| | AADDAWTFSANRYKKATGPKWGKISVSNLLYDANQANAEL |
| | ISQSISKYLSKQKDNQGCEGRKMKFLIKIIEPLRENFVKH |
| | TRWLHEMTQKDCEVRAQFSRVSM |
| SEQ | FPSDVGADALKHVRMLQPRLTDEVRKVALTRAPSDRPALA |
| ID | RFAAVAQDGLAFVRHLNVSANHDSNCTFPRDPRDPRRGPC |
| NO: | EPNPCAFLREVWGFRIVARGNERALSYRRGLAGCKSCVQS |
| 127 | TGFPSVPFHRIGADDCMRKLHEILKARNWRLLARNIGRER |
| | EADPLLTELSEYLLVDARTYPDGAAPNSGRLAENVIKRAA |
| | KKFRDEGMRDIHAQLRVHSREGKVPKGRLQRLRRIERKHR |
| | AIHALDPGPSWEAEGSARAEVQGVAVYRSQLLRVGHHTQQ |
| | IEPVGIVARTLFGVGRTDLDVAVSVLGAPLTKRKKGSKTL |
| | ESTEDFRIAKARETRAEDKIEVAFVLYPTASLLRDEIPKD |
| | AFPAMRIDRFLLKVGSVQADREILLQDDYYRFGDAEVKAG |
| | KNKGRTVTRPVKVPRLQALRPDAKFRVNVWADPFGAGDSP |
| | GTLLRLEVSGVTRRSQPLRLLRYGQPSTQPANFLCWRPHR |
| | VPDPMTFTPRQKFGERRKNRRTRRPRVFERLYQVHIKHLA |
| | HLEPNRKWFEEARVSAQKWAKARAIRRKGAEDIPVVAPPA |
| | KRRWAALQPNAELWDLYAHDREARKRFRGGRAAEGEEFKP |
| | RLNLYLAHEPEAEWESKRDRWERYEKKWTAVLEEHSRMCA |
| | VADRTLPQFLSDPLGARMDDKDYAFVGKSALAVAEAFVEE |
| | GTVERAQGNCSITAKKKFASNASRKRLSVANLLDVSDKAD |
| | RALVFQAVRQYVQRQAENGGVEGRRMAFLRKLLAPLRQNF |
| | VCHTRWLHM |
| SEQ | AARKKKRGKIGITVKAKEKSPPAAGPFMARKLVNVAANVD |
| ID | GVEVHLCVECEADAHGSASARLLGGCRSCTGSIGAEGRLM |
| NO: | GSVDVDRERVIAEPVHTETERLGPDVKAFEAGTAESKYAI |
| 128 | QRGLEYWGVDLISRNRARTVRKMEEADRPESSTMEKTSWD |
| | EIAIKTYSQAYHASENHLFWERQRRVRQHALALFRRARER |
| | NRGESPLQSTQRPAPLVLAALHAEAAAISGRARAEYVLRG |
| | PSANVRAAAADIDAKPLGHYKTPSPKVARGFPVKRDLLRA |
| | RHRIVGLSRAYFKPSDVVRGTSDAIAHVAGRNIGVAGGKP |
| | KEIEKTFTLPFVAYWEDVDRVVHCSSFKADGPWVRDQRIK |
| | IRGVSSAVGTFSLYGLDVAWSKPTSFYIRCSDIRKKFHPK |
| | GFGPMKHWRQWAKELDRLTEQRASCVVRALQDDEELLQTM |
| | ERGQRYYDVFSCAATHATRGEADPSGGCSRCELVSCGVAH |
| | KVTKKAKGDTGIEAVAVAGCSLCESKLVGPSKPRVHRQMA |
| | ALRQSHALNYLRRLQREWEALEAVQAPTPYLRFKYARHLE |
| | VRSM |
| SEQ | AAKKKKQRGKIGISVKPKEGSAPPADGPFMARKLVNVAAN |
| ID | VDGVEVNLCIECEADAHGSAPARLLGGCKSCTGSIGAEGR |
| NO | LMGSVDVDRADAIAKPVNTETEKLGPDVQAFEAGTAETKY |
| 129 | ALQRGLEYWGVDLISRNRSRTVRRTEEGQPESATMEKTSW |
| | DEIAIKSYTRAYHASENHLFWERQRRVRQHALALFKRAKE |
| | RNRGDSTLPREPGHGLVAIAALACEAYAVGGRNLAETVVR |
| | GPTFGTARAVRDVEIASLGRYKTPSPKVAHGSPVKRDFLR |
| | ARHRIVGLARAYYRPSDVVRGTSDAIAHVAGRNIGVAGGK |
| | PRAVEAVFTLPFVAYWEDVDRVVHCSSFQVSAPWNRDQRM |
| | KIAGVTTAAGTFSLHGGELKWAKPTSFYIRCSDTRRKFRP |
| | KGFGPMKRWRQWAKDLDRLVEQRASCVVRALQDDAALLET |
| | MERGQRYYDVFACAVTHATRGEADRLAGCSRCALTPCQEA |
| | HRVTTKPRGDAGVEQVQTSDCSLCEGKLVGPSKPRLHRTL |
| | TLLRQEHGLNYLRRLQREWESLEAVQVPTPYLRFKYARHL |
| | EVRSM |
| SEQ | TDSQSESVPEVVYALTGGEVPGRVPPDGGSAEGARNAPTG |
| ID | LRKQRGKIKISAKPSKPGSPASSLARTLVNEAANVDGVQS |
| NO: | SGCATCRMRANGSAPRALPIGCVACASSIGRAPQEETVCA |
| 130 | LPTTQGPDVRLLEGGHALRKYDIQRALEYWGVDLIGRNLD |
| | RQAGRGMEPAEGATATMKRVSMDELAVLDFGKSYYASEQH |
| | LFAARQRRVRQHAKALKIRAKHANRSGSVKRALDRSRKQV |
| | TALAREFFKPSDVVRGDSDALAHVVGRNLGVSRHPAREIP |
| | QTFTLPLCAYWEDVDRVISCSSLLAGEPFARDQEIRIEGV |
| | SSALGSLRLYRGAIEWHKPTSLYIRCSDTRRKFRPRGGLK |
| | KRWRQWAKDLDRLVEQRACCIVRSLQADVELLQTMERAQR |
| | FYDVHDCAATHVGPVAVRCSPCAGKQFDWDRYRLLAALRQ |
| | EHALNYLRRLQREWESLEAQQVKMPYLRFKYARKLEVSGP |
| | LIGLEVRREPSMGTAIAEM |
| SEQ | AGTAGRRHGSLGARRSINIAGVTDRHGRWGCESCVYTRDQ |
| ID | AGNRARCAPCDQSTYAPDVQEVTIGQRQAKYTIFLTLQSF |
| NO: | SWTNTMRNNKRAAAGRSKRTTGKRIGQLAEIKITGVGLAH |
| 131 | AHNVIQRSLQHNITKMWRAEKGKSKRVARLKKAKQLTKRR |
| | AYFRRRMSRQSRGNGFFRTGKGGIHAVAPVKIGLDVGMIA |
| | SGSSEPADEQTVTLDAIWKGRKKKIRLIGAKGELAVAACR |
| | FREQQTKGDKCIPLILQDGEVRWNQNNWQCHPKKLVPLCG |
| | LEVSRKFVSQADRLAQNKVASPLAARFDKTSVKGTLVESD |
| | FAAVLVNVTSIYQQCHAMLLRSQEPTPSLRVQRTITSM |
| SEQ | GVRFSPAQSQVFFRTVIPQSVEARFAINMAAIHDAAGAFG |
| ID | CSVCRFEDRTPRNAKAVHGCSPCTRSTNRPDVFVLPVGAI |
| NO: | KAKYDVFMRLLGFNWTHLNRRQAKRVTVRDRIGQLDELAI |
| 132 | SMLTGKAKAVLKKSICHNVDKSFKAMRGSLKKLHRKASKT |
| | GKSQLRAKLSDLRERTNTTQEGSHVEGDSDVALNKIGLDV |
| | GLVGKPDYPSEESVEVVVCLYFVGKVLILDAQGRIRDMRA |
| | KQYDGFKIPIIQRGQLTVLSVKDLGKWSLVRQDYVLAGDL |
| | RFEPKISKDRKYAECVKRIALITLQASLGFKERIPYYVTK |
| | QVEIKNASHIAFVTEAIQNCAENFREMTEYLMKYQEKSPD |
| | LKVLLTQLM |
| SEQ | RAVVGKVFLEQARRALNLATNFGTNHRTGCNGCYVTPGKL |
| ID | SIPQDGEKNAAGCTSCLMKATASYVSYPKPLGEKVAKYST |
| NO: | LDALKGFPWYSLRLNLRPNYRGKPINGVQEVAPVSKFRLA |
| 133 | EEVIQAVQRYHFTELEQSFPGGRRRLRELRAFYTKEYRRA |
| | PEQRQHVVNGDRNIVVVTVLHELGFSVGMFNEVELLPKTP |
| | IECAVNVFIRGNRVLLEVRKPQFDKERLLVESLWKKDSRR |
| | HTAKWTPPNNEGRIFTAEGWKDFQLPLLLGSTSRSLRAIE |
| | KEGFVQLAPGRDPDYNNTIDEQHSGRPFLPLYLYLQGTIS |
| | QEYCVFAGTWVIPFQDGISPYSTKDTFQPDLKRKAYSLLL |
| | DAVKHRLGNKVASGLQYGRFPAIEELKRLVRMHGATRKIP |
| | RGEKDLLKKGDPDTPEWWLLEQYPEFWRLCDAAAKRVSQN |
| | VGLLLSLKKQPLWQRRWLESRTRNEPLDNLPLSMALTLHL |
| | TNEEAL |
| SEQ | AAVYSKFYIENHFKMGIPETLSRIRGPSIIQGFSVNENYI |
| ID | NIAGVGDRDFIFGCKKCKYTRGKPSSKKINKCHPCKRSTY |
| NO: | PEPVIDVRGSISEFKYKIYNKLKQEPNQSIKQNTKGRMNP |
| 134 | SDHTSSNDGIIINGIDNRIAYNVIFSSYKHLMEKQINLLR |
| | DTTKRKARQIKKYNNSGKKKHSLRSQTKGNLKNRYHMLGM |
| | FKKGSLTITNEGDFITAVRKVGLDISLYKNESLNKQEVET |
| | ELCLNIKWGRTKSYTVSGYIPLPINIDWKLYLFEKETGLT |
| | LRLFGNKYKIQSKKFLIAQLFKPKRPPCADPVVKKAQKWS |
| | ALNAHVQQMAGLFSDSHLLKRELKNRMHKQLDFKSLWVGT |
| | EDYIKWFEELSRSYVEGAEKSLEFFRQDYFCFNYTKQTTM |
| SEQ | PQQQRDLMLMAANYDQDYGNGCGPCTVVASAAYRPDPQAQ |
| ID | HGCKRHLRTLGASAVTHVGLGDRTATITALHRLRGPAALA |
| NO: | ARARAAQAASAPMTPDTDAPDDRRRLEAIDADDVVLVGAH |
| 135 | RALWSAVRRWADDRRAALRRRLHSEREWLLKDQIRWAELY |
| | TLIEASGTPPQGRWRNTLGALRGQSRWRRVLAPTMRATCA |
| | ETHAELWDALAELVPEMAKDRRGLLRPPVEADALWRAPMI |
| | VEGWRGGHSVVVDAVAPPLDLPQPCAWTAVRLSGDPRQRW |
| | GLHLAVPPLGQVQPPDPLKATLAVSMRHRGGVRVRTLQAM |
| | AVDADAPMQRHLQVPLTLQRGGGLQWGIHSRGVRRREARS |
| | MASWEGPPIWTGLQLVNRWKGQGSALLAPDRPPDTPPYAP |
| | DAAVAPAQPDTKRARRTLKEACTVCRCAPGHMRQLQVTLT |
| | GDGTWRRFRLRAPQGAKRKAEVLKVATQHDERIANYTAWY |
| | LKRPEHAAGCDTCDGDSRLDGACRGCRPLLVGDQCFRRYL |
| | DKIEADRDDGLAQIKPKAQEAVAAMAAKRDARAQKVAARA |
| | AKLSEATGQRTAATRDASHEARAQKELEAVATEGTTVRHD |
| | AAAVSAFGSWVARKGDEYRHQVGVLANRLEHGLRLQELMA |
| | PDSVVADQQRASGHARVGYRYVLTAM |
| SEQ | AVAHPVGRGNAGSPGARGPEELPRQLVNRASNVTRPATYG |
| ID | CAPCRHVRLSIPKPVLTGCRACEQTTHPAPKRAVRGGADA |
| NO: | AKYDLAAFFAGWAADLEGRNRRRQVHAPLDPQPDPNHEPA |
| 136 | VTLQKIDLAEVSIEEFQRVLARSVKHRHDGRASREREKAR |
| | AYAQVAKKRRNSHAHGARTRRAVRRQTRAVRRAHRMGANS |
| | GEILVASGAEDPVPEAIDHAAQLRRRIRACARDLEGLRHL |
| | SRRYLKTLEKPCRRPRAPDLGRARCHALVESLQAAERELE |
| | ELRRCDSPDTAMRRLDAVLAAAASTDATFATGWTVVGMDL |
| | GVAPRGSAAPEVSPMEMAISV |
| | FWRKGSRRVIVSKPIAGMPIRRHELIRLEGLGTLRLDGNH |
| | YTGAGVTKGRGLSEGTEPDFREKSPSTLGFTLSDYRHESR |
| | WRPYGAKQGKTARQFFAAMSRELRALVEHQVLAPMGPPLL |
| | EAHERRFETLLKGQDNKSIHAGGGGRYVWRGPPDSKKRPA |
| | ADGDWFRFGRGHADHRGWANKRHELAANYLQSAFRLWSTL |
| | AEAQEPTPYARYKYTRVTM |
| SEQ | WDFLTLQVYERHTSPEVCVAGNSTKCASGTRKSDHTHGVG |
| ID | VKLGAQEINVSANDDRDHEVGCNICVISRVSLDIKGWRYG |
| NO: | CESCVQSTPEWRSIVRFDRNHKEAKGECLSRFEYWGAQSI |
| 137 | ARSLKRNKLMGGVNLDELAIVQNENVVKTSLKHLFDKRKD |
| | RIQANLKAVKVRMRERRKSGRQRKALRRQCRKLKRYLRSY |
| | DPSDIKEGNSCSAFTKLGLDIGISPNKPPKIEPKVEVVFS |
| | LFYQGACDKIVTVSSPESPLPRSWKIKIDGIRALYVKSTK |
| | VKFGGRTFRAGQRNNRRKVRPPNVKKGKRKGSRSQFFNKF |
| | AVGLDAVSQQLPIASVQGLWGRAETKKAQTICLKQLESNK |
| | PLKESQRCLFLADNWVVRVCGFLRALSQRQGPTPYIRYRY |
| | RCNM |
| SEQ | ARNVGQRNASRQSKRESAKARSRRVTGGHASVTQGVALIN |
| ID | AAANADRDHTTGCEPCTWERVNLPLQEVIHGCDSCTKSSP |
| NO: | FWRDIKVVNKGYREAKEEIMRIASGISADHLSRALSHNKV |
| 138 | MGRLNLDEVCILDFRTVLDTSLKHLTDSRSNGIKEHIRAV |
| | HRKIRMRRKSGKTARALRKQYFALRRQWKAGHKPNSIREG |
| | NSLTALRAVGFDVGVSEGTEPMPAPQTEVVLSVFYKGSAT |
| | RILRISSPHPIAKRSWKVKIAGIKALKLIRREHDFSFGRE |
| | TYNASQRAEKRKFSPHAARKDFFNSFAVQLDRLAQQLCVS |
| | SVENLWVTEPQQKLLTLAKDTAPYGIREGARFADTRARLA |
| | WNWVFRVCGFTRALHQEQEPTPYCRFTWRSKM |

In some embodiments, the Type V CRISPR/Cas enzyme is a Case nuclease. A Case polypeptide can function as an endonuclease that catalyzes cleavage at a specific sequence in a target nucleic acid. A programmable Case nuclease of the present disclosure can have a single active site in a RuvC domain that is capable of catalyzing pre-crRNA processing and nicking or cleaving of nucleic acids. This compact catalytic site can render the programmable Case nuclease especially advantageous for genome engineering and new functionalities for genome manipulation.

TABLE 4 provides amino acid sequences of illustrative Case polypeptides that can be used in compositions and methods of the disclosure.

TABLE 4
CasΦ Amino Acid Sequences

| | SEQ ID | |
|---|---|---|
| Name | NO | Amino Acid Sequence |
| CasΦ.1 | SEQ ID | MADTPTLFTQFLRHHLPGQRFRKDILKQAG |
| | NO: 139 | RILANKGEDATIAFLRGKSEESPPDFQPPV |
| | | KCPIIACSRPLTEWPIYQASVAIQGYVYGQ |
| | | SLAEFEASDPGCSKDGLLGWFDKTGVCTDY |
| | | FSVQGLNLIFQNARKRYIGVQTKVTNRNEK |
| | | RHKKLKRINAKRIAEGLPELTSDEPESALD |
| | | ETGHLIDPPGLNTNIYCYQQVSPKPLALSE |
| | | VNQLPTAYAGYSTSGDDPIQPMVTKDRLSI |
| | | SKGQPGYIPEHQRALLSQKKHRRMRGYGLK |
| | | ARALLVIVRIQDDWAVIDLRSLLRNAYWRR |
| | | IVQTKEPSTITKLLKLVTGDPVLDATRMVA |
| | | TFTYKPGIVQVRSAKCLKNKQGSKLFSERY |
| | | LNETVSVTSIDLGSNNLVAVATYRLVNGNT |
| | | PELLQRFTLPSHLVKDFERYKQAHDTLEDS |
| | | IQKTAVASLPQGQQTEIRMWSMYGFREAQE |
| | | RVCQELGLADGSIPWNVMTATSTILTDLFL |
| | | ARGGDPKKCMFTSEPKKKKNSKQVLYKIRD |
| | | RAWAKMYRTLLSKETREAWNKALWGLKRGS |
| | | PDYARLSKRKEELARRCVNYTISTAEKRAQ |
| | | CGRTIVALEDLNIGFFHGRGKQEPGWVGLF |
| | | TRKKENRWLMQALHKAFLELAHHRGYHVIE |
| | | VNPAYTSQTCPVCRHCDPDNRDQHNREAFH |
| | | CIGCGFRGNADLDVATHNIAMVAITGESLK |
| | | RARGSVASKTPQPLAAE |
| CasΦ.2 | SEQ ID | MPKPAVESEFSKVLKKHFPGERFRSSYMKR |
| | NO: 140 | GGKILAAQGEEAVVAYLQGKSEEEPPNFQP |
| | | PAKCHVVTKSRDFAEWPIMKASEAIQRYIY |
| | | ALSTTERAACKPGKSSESHAAWFAATGVSN |
| | | HGYSHVQGLNLIFDHTLGRYDGVLKKVQLR |
| | | NEKARARLESINASRADEGLPEIKAEEEEV |
| | | ATNETGHLLQPPGINPSFYVYQTISPQAYR |
| | | PRDEIVLPPEYAGYVRDPNAPIPLGVVRNR |
| | | CDIQKGCPGYIPEWQREAGTAISPKTGKAV |
| | | TVPGLSPKKNKRMRRYWRSEKEKAQDALLV |
| | | TVRIGTDWVVIDVRGLLRNARWRTIAPKDI |
| | | SLNALLDLFTGDPVIDVRRNIVTFTYTLDA |
| | | CGTYARKWTLKGKQTKATLDKLTATQTVAL |
| | | VAIDLGQTNPISAGISRVTQENGALQCEPL |
| | | DRFTLPDDLLKDISAYRIAWDRNEEELRAR |
| | | SVEALPEAQQAEVRALDGVSKETARTQLCA |
| | | DFGLDPKRLPWDKMSSNTTFISEALLSNSV |
| | | SRDQVFFTPAPKKGAKKKAPVEVMRKDRTW |
| | | ARAYKPRLSVEAQKLKNEALWALKRTSPEY |
| | | LKLSRRKEELCRRSINYVIEKTRRRTQCQI |
| | | VIPVIEDLNVRFFHGSGKRLPGWDNFFTAK |
| | | KENRWFIQGLHKAFSDLRTHRSFYVFEVRP |
| | | ERTSITCPKCGHCEVGNRDGEAFQCLSCGK |
| | | TCNADLDVATHNLTQVALTGKTMPKREEPR |
| | | DAQGTAPARKTKKASKSKAPPAEREDQTPA |
| | | QEPSQTS |
| CasΦ.3 | SEQ ID | MYILEMADLKSEPSLLAKLLRDRFPGKYWL |
| | NO: 141 | PKYWKLAEKKRLTGGEEAACEYMADKQLDS |
| | | PPPNFRPPARCVILAKSRPFEDWPVHRVAS |
| | | KAQSFVIGLSEQGFAALRAAPPSTADARRD |
| | | WLRSHGASEDDLMALEAQLLETIMGNAISL |
| | | HGGVLKKIDNANVKAAKRLSGRNEARLNKG |
| | | LQELPPEQEGSAYGADGLLVNPPGLNLNIY |
| | | CRKSCCPKPVKNTARFVGHYPGYLRDSDSI |
| | | LISGTMDRLTIIEGMPGHIPAWQREQGLVK |
| | | PGGRRRRLSGSESNMRQKVDPSTGPRRSTR |
| | | SGTVNRSNQRTGRNGDPLLVEIRMKEDWVL |
| | | LDARGLLRNLRWRESKRGLSCDHEDLSLSG |
| | | LLALFSGDPVIDPVRNEVVFLYGEGIIPVR |
| | | STKPVGTRQSKKLLERQASMGPLTLISCDL |
| | | GQTNLIAGRASAISLTHGSLGVRSSVRIEL |
| | | DPEIIKSFERLRKDADRLETEILTAAKETL |
| | | SDEQRGEVNSHEKDSPQTAKASLCRELGLH |
| | | PPSLPWGQMGPSTTFIADMLISHGRDDDAF |
| | | LSHGEFPTLEKRKKFDKRFCLESRPLLSSE |
| | | TRKALNESLWEVKRTSSEYARLSQRKKEMA |
| | | RRAVNFVVEISRRKTGLSNVIVNIEDLNVR |
| | | IFHGGGKQAPGWDGFFRPKSENRWFIQAIH |
| | | KAFSDLAAHHGIPVIESDPQRTSMTCPECG |
| | | HCDSKNRNGVRFLCKGCGASMDADFDAACR |
| | | NLERVALTGKPMPKPSTSCERLLSATTGKV |
| | | CSDHSLSHDAIEKAS |
| CasΦ.4 | SEQ ID | MEKEITELTKIRREFPNKKFSSTDMKKAGK |
| | NO: 142 | LLKAEGPDAVRDFLNSCQEIIGDFKPPVKT |
| | | NIVSISRPFEEWPVSMVGRAIQEYYFSLTK |
| | | EELESVHPGTSSEDHKSFFNITGLSNYNYT |
| | | SVQGLNLIFKNAKAIYDGTLVKANNKNKKL |
| | | EKKFNEINHKRSLEGLPIITPDFEEPFDEN |
| | | GHLNNPPGINRNIYGYQGCAAKVFVPSKHK |
| | | MVSLPKEYEGYNRDPNLSLAGFRNRLEIPE |
| | | GEPGHVPWFQRMDIPEGQIGHVNKIQRFNF |
| | | VHGKNSGKVKFSDKTGRVKRYHHSKYKDAT |
| | | KPYKFLEESKKVSALDSILAIITIGDDWVV |
| | | FDIRGLYRNVFYRELAQKGLTAVQLLDLFT |
| | | GDPVIDPKKGVVTFSYKEGVVPVFSQKIVP |
| | | RFKSRDTLEKLTSQGPVALLSVDLGQNEPV |
| | | AARVCSLKNINDKITLDNSCRISFLDDYKK |
| | | QIKDYRDSLDELEIKIRLEAINSLETNQQV |
| | | EIRDLDVFSADRAKANTVDMFDIDPNLISW |
| | | DSMSDARVSTQISDLYLKNGGDESRVYFEI |
| | | NNKRIKRSDYNISQLVRPKLSDSTRKNLND |
| | | SIWKLKRTSEEYLKLSKRKLELSRAVVNYT |
| | | IRQSKLLSGINDIVIILEDLDVKKKFNGRG |
| | | IRDIGWDNFFSSRKENRWFIPAFHKAFSEL |
| | | SSNRGLCVIEVNPAWTSATCPDCGFCSKEN |
| | | RDGINFTCRKCGVSYHADIDVATLNIARVA |
| | | VLGKPMSGPADRERLGDTKKPRVARSRKTM |
| | | KRKDISNSTVEAMVTA |
| CasΦ.5 | SEQ ID | MDMLDTETNYATETPAQQQDYSPKPPKKAQ |
| | NO: 143 | RAPKGFSKKARPEKKPPKPITLFTQKHFSG |
| | | VRFLKRVIRDASKILKLSESRTITFLEQAI |
| | | ERDGSAPPDVTPPVHNTIMAVTRPFEEWPE |
| | | VILSKALQKHCYALTKKIKIKTWPKKGPGK |
| | | KCLAAWSARTKIPLIPGQVQATNGLFDRIG |
| | | SIYDGVEKKVTNRNANKKLEYDEAIKEGRN |
| | | PAVPEYETAYNIDGTLINKPGYNPNLYITQ |
| | | SRTPRLITEADRPLVEKILWQMVEKKTQSR |
| | | NQARRARLEKAAHLQGLPVPKFVPEKVDRS |
| | | QKIEIRIIDPLDKIEPYMPQDRMAIKASQD |
| | | GHVPYWQRPFLSKRRNRRVRAGWGKQVSSI |
| | | QAWLTGALLVIVRLGNEAFLADIRGALRNA |
| | | QWRKLLKPDATYQSLFNLFTGDPVVNTRTN |
| | | HLTMAYREGVVNIVKSRSFKGRQTREHLLT |
| | | LLGQGKTVAGVSFDLGQKHAAGLLAAHFGL |
| | | GEDGNPVFTPIQACFLPQRYLDSLTNYRNR |
| | | YDALTLDMRRQSLLALTPAQQQEFADAQRD |
| | | PGGQAKRACCLKLNLNPDEIRWDLVSGIST |
| | | MISDLYIERGGDPRDVHQQVETKPKGKRKS |
| | | EIRILKIRDGKWAYDFRPKIADETRKAQRE |
| | | QLWKLQKASSEFERLSRYKINIARAIANWA |
| | | LQWGRELSGCDIVIPVLEDLNVGSKFFDGK |
| | | GKWLLGWDNRFTPKKENRWFIKVLHKAVAE |
| | | LAPHRGVPVYEVMPHRTSMTCPACHYCHPT |
| | | NREGDRFECQSCHVVKNTDRDVAPYNILRV |
| | | AVEGKTLDRWQAEKKPQAEPDRPMILIDNQ |
| | | ES |
| CasΦ.6 | SEQ ID | MDMLDTETNYATETPAQQQDYSPKPPKKAQ |
| | NO: 144 | RAPKGFSKKARPEKKPPKPITLFTQKHFSG |
| | | VRFLKRVIRDASKILKLSESRTITFLEQAI |
| | | ERDGSAPPDVTPPVHNTIMAVTRPFEEWPE |
| | | VILSKALQKHCYALTKKIKIKTWPKKGPGK |
| | | KCLAAWSARTKIPLIPGQVQATNGLFDRIG |
| | | SIYDGVEKKVTNRNANKKLEYDEAIKEGRN |
| | | PAVPEYETAYNIDGTLINKPGYNPNLYITQ |
| | | SRTPRLITEADRPLVEKILWQMVEKKTQSR |
| | | NQARRARLEKAAHLQGLPVPKFVPEKVDRS |
| | | QKIEIRIIDPLDKIEPYMPQDRMAIKASQD |
| | | GHVPYWQRPFLSKRRNRRVRAGWGKQVSSI |
| | | QAWLTGALLVIVRLGNEAFLADIRGALRNA |
| | | QWRKLLKPDATYQSLFNLFTGDPVVNTRTN |
| | | HLTMAYREGVVDIVKSRSFKGRQTREHLLT |
| | | LLGQGKTVAGVSFDLGQKHAAGLLAAHFGL |
| | | GEDGNPVFTPIQACFLPQRYLDSLTNYRNR |
| | | YDALTLDMRRQSLLALTPAQQQEFADAQRD |
| | | PGGQAKRACCLKLNLNPDEIRWDLVSGIST |
| | | MISDLYIERGGDPRDVHQQVETKPKGKRKS |
| | | EIRILKIRDGKWAYDFRPKIADETRKAQRE |
| | | QLWKLQKASSEFERLSRYKINIARAIANWA |
| | | LQWGRELSGCDIVIPVLEDLNVGSKFFDGK |
| | | GKWLLGWDNRFTPKKENRWFIKVLHKAVAE |
| | | LAPHKGVPVYEVMPHRTSMTCPACHYCHPT |
| | | NREGDRFECQSCHVVKNTDRDVAPYNILRV |
| | | AVEGKTLDRWQAEKKPQAEPDRPMILIDNQ |
| | | ES |
| CasΦ.7 | SEQ ID | MSSLPTPLELLKQKHADLFKGLQFSSKDNK |
| | NO: 145 | MAGKVLKKDGEEAALAFLSERGVSRGELPN |
| | | FRPPAKTLVVAQSRPFEEFPIYRVSEAIQL |
| | | YVYSLSVKELETVPSGSSTKKEHQRFFQDS |
| | | SVPDFGYTSVQGLNKIFGLARGIYLGVITR |
| | | GENQLQKAKSKHEALNKKRRASGEAETEFD |
| | | PTPYEYMTPERKLAKPPGVNHSIMCYVDIS |
| | | VDEFDFRNPDGIVLPSEYAGYCREINTAIE |
| | | KGTVDRLGHLKGGPGYIPGHQRKESTTEGP |
| | | KINFRKGRIRRSYTALYAKRDSRRVRQGKL |
| | | ALPSYRHHMMRLNSNAESAILAVIFFGKDW |
| | | VVFDLRGLLRNVRWRNLFVDGSTPSTLLGM |
| | | FGDPVIDPKRGVVAFCYKEQIVPVVSKSIT |
| | | KMVKAPELLNKLYLKSEDPLVLVAIDLGQT |
| | | NPVGVGVYRVMNASLDYEVVTRFALESELL |
| | | REIESYRQRTNAFEAQIRAETFDAMTSEEQ |
| | | EEITRVRAFSASKAKENVCHRFGMPVDAVD |
| | | WATMGSNTIHIAKWVMRHGDPSLVEVLEYR |
| | | KDNEIKLDKNGVPKKVKLTDKRIANLTSIR |
| | | LRFSQETSKHYNDTMWELRRKHPVYQKLSK |
| | | SKADFSRRVVNSIIRRVNHLVPRARIVFII |
| | | EDLKNLGKVFHGSGKRELGWDSYFEPKSEN |
| | | RWFIQVLHKAFSETGKHKGYYIIECWPNWT |
| | | SCTCPKCSCCDSENRHGEVFRCLACGYTCN |
| | | TDFGTAPDNLVKIATTGKGLPGPKKRCKGS |
| | | SKGKNPKIARSSETGVSVTESGAPKVKKSS |
| | | PTQTSQSSSQSAP |
| CasΦ.8 | SEQ ID | MNKIEKEKTPLAKLMNENFAGLRFPFAIIK |
| | NO: 146 | QAGKKLLKEGELKTIEYMTGKGSIEPLPNF |
| | | KPPVKCLIVAKRRDLKYFPICKASCEIQSY |
| | | VYSLNYKDFMDYFSTPMTSQKQHEEFFKKS |
| | | GLNIEYQNVAGLNLIFNNVKNTYNGVILKV |
| | | KNRNEKLKKKAIKNNYEFEEIKTFNDDGCL |
| | | INKPGINNVIYCFQSISPKILKNITHLPKE |
| | | YNDYDCSVDRNIIQKYVSRLDIPESQPGHV |
| | | PEWQRKLPEFNNTNNPRRRRKWYSNGRNIS |
| | | KGYSVDQVNQAKIEDSLLAQIKIGEDWIIL |
| | | DIRGLLRDLNRRELISYKNKLTIKDVLGFF |
| | | SDYPIIDIKKNLVTFCYKEGVIQVVSQKSI |
| | | GNKKSKQLLEKLIENKPIALVSIDLGQTNP |
| | | VSVKISKLNKINNKISIESFTYRFLNEEIL |
| | | KEIEKYRKDYDKLELKLINEA |
| CasΦ.9 | SEQ ID | MDMLDTETNYATETPSQQQDYSPKPPKKDR |
| | NO: 147 | RAPKGFSKKARPEKKPPKPITLFTQKHFSG |
| | | VRFLKRVIRDASKILKLSESRTITFLEQAI |
| | | ERDGSAPPDVTPPVHNTIMAVTRPFEEWPE |
| | | VILSKALQKHCYALTKKIKIKTWPKKGPGK |
| | | KCLAAWSARTKIPLIPGQVQATNGLFDRIG |
| | | SIYDGVEKKVTNRNANKKLEYDEAIKEGRN |
| | | PAVPEYETAYNIDGTLINKPGYNPNLYITQ |
| | | SRTPRLITEADRPLVEKILWQMVEKKTQSR |
| | | NQARRARLEKAAHLQGLPVPKFVPEKVDRS |
| | | QKIEIRIIDPLDKIEPYMPQDRMAIKASQD |
| | | GHVPYWQRPFLSKRRNRRVRAGWGKQVSSI |
| | | QAWLTGALLVIVRLGNEAFLADIRGALRNA |
| | | QWRKLLKPDATYQSLFNLFTGDPVVNTRTN |
| | | HLTMAYREGVVDIVKSRSFKGRQTREHLLT |
| | | LLGQGKTVAGVSFDLGQKHAAGLLAAHFGL |
| | | GEDGNPVFTPIQACFLPQRYLDSLTNYRNR |
| | | YDALTLDMRRQSLLALTPAQQQEFADAQRD |
| | | PGGQAKRACCLKLNLNPDEIRWDLVSGIST |
| | | MISDLYIERGGDPRDVHQQVETKPKGKRKS |
| | | EIRILKIRDGKWAYDFRPKIADETRKAQRE |
| | | QLWKLQKASSEFERLSRYKINIARAIANWA |
| | | LQWGRELSGCDIVIPVLEDLNVGSKFFDGK |
| | | GKWLLGWDNRFTPKKENRWFIKVLHKAVAE |
| | | LAPHRGVPVYEVMPHRTSMTCPACHYCHPT |
| | | NREGDRFECQSCHVVKNTDRDVAPYNILRV |
| | | AVEGKTLDRWQAEKKPQAEPDRPMILIDNQ |
| | | ES |
| CasΦ.10 | SEQ ID | MDMLDTETNYATETPSQQQDYSPKPPKKDR |
| | NO: 148 | RAPKGFSKKARPEKKPPKPITLFTQKHFSG |
| | | VRFLKRVIRDASKILKLSESRTITFLEQAI |
| | | ERDGSAPPDVTPPVHNTIMAVTRPFEEWPE |
| | | VILSKALQKHCYALTKKIKIKTWPKKGPGK |
| | | KCLAAWSARTKIPLIPGQVQATNGLFDRIG |
| | | SIYDGVEKKVTNRNANKKLEYDEAIKEGRN |
| | | PAVPEYETAYNIDGTLINKPGYNPNLYITQ |
| | | SRTPRLITEADRPLVEKILWQMVEKKTQSR |
| | | NQARRARLEKAAHLQGLPVPKFVPEKVDRS |
| | | QKIEIRIIDPLDKIEPYMPQDRMAIKASQD |
| | | GHVPYWQRPFLSKRRNRRVRAGWGKQVSSI |
| | | QAWLTGALLVIVRLGNEAFLADIRGALRNA |
| | | QWRKLLKPDATYQSLFNLFTGDPVVNTRTN |
| | | HLTMAYREGVVNIVKSRSFKGRQTREHLLT |
| | | LLGQGKTVAGVSFDLGQKHAAGLLAAHFGL |
| | | GEDGNPVFTPIQACFLPQRYLDSLTNYRNR |
| | | YDALTLDMRRQSLLALTPAQQQEFADAQRD |
| | | PGGQAKRACCLKLNLNPDEIRWDLVSGIST |
| | | MISDLYIERGGDPRDVHQQVETKPKGKRKS |
| | | EIRILKIRDGKWAYDFRPKIADETRKAQRE |
| | | QLWKLQKASSEFERLSRYKINIARAIANWA |
| | | LQWGRELSGCDIVIPVLEDLNVGSKFFDGK |
| | | GKWLLGWDNRFTPKKENRWFIKVLHKAVAE |
| | | LAPHRGVPVYEVMPHRTSMTCPACHYCHPT |
| | | NREGDRFECQSCHVVKNTDRDVAPYNILRV |
| | | AVEGKTLDRWQAEKKPQAEPDRPMILIDNQ |
| | | ES |
| CasΦ.11 | SEQ ID | MSNKTTPPSPLSLLLRAHFPGLKFESQDYK |
| | NO: 149 | IAGKKLRDGGPEAVISYLTGKGQAKLKDVK |
| | | PPAKAFVIAQSRPFIEWDLVRVSRQIQEKI |
| | | FGIPATKGRPKQDGLSETAFNEAVASLEVD |
| | | GKSKLNEETRAAFYEVLGLDAPSLHAQAQN |
| | | ALIKSAISIREGVLKKVENRNEKNLSKTKR |
| | | RKEAGEEATFVEEKAHDERGYLIHPPGVNQ |
| | | TIPGYQAVVIKSCPSDFIGLPSGCLAKESA |
| | | EALTDYLPHDRMTIPKGQPGYVPEWQHPLL |
| | | NRRKNRRRRDWYSASLNKPKATCSKRSGTP |
| | | NRKNSRTDQIQSGRFKGAIPVLMRFQDEWV |
| | | IIDIRGLLRNARYRKLLKEKSTIPDLLSLF |
| | | TGDPSIDMRQGVCTFIYKAGQACSAKMVKT |
| | | KNAPEILSELTKSGPVVLVSIDLGQTNPIA |
| | | AKVSRVTQLSDGQLSHETLLRELLSNDSSD |
| | | GKEIARYRVASDRLRDKLANLAVERLSPEH |
| | | KSEILRAKNDTPALCKARVCAALGLNPEMI |
| | | AWDKMTPYTEFLATAYLEKGGDRKVATLKP |
| | | KNRPEMLRRDIKFKGTEGVRIEVSPEAAEA |
| | | YREAQWDLQRTSPEYLRLSTWKQELTKRIL |
| | | NQLRHKAAKSSQCEVVVMAFEDLNIKMMHG |
| | | NGKWADGGWDAFFIKKRENRWFMQAFHKSL |
| | | TELGAHKGVPTIEVTPHRTSITCTKCGHCD |
| | | KANRDGERFACQKCGFVAHADLEIATDNIE |
| | | RVALTGKPMPKPESERSGDAKKSVGARKAA |
| | | FKPEEDAEAAE |
| CasΦ.12 | SEQ ID | MIKPTVSQFLTPGFKLIRNHSRTAGLKLKN |
| | NO: 150 | EGEEACKKFVRENEIPKDECPNFQGGPAIA |
| | | NIIAKSREFTEWEIYQSSLAIQEVIFTLPK |
| | | DKLPEPILKEEWRAQWLSEHGLDTVPYKEA |
| | | AGLNLIIKNAVNTYKGVQVKVDNKNKNNLA |
| | | KINRKNEIAKLNGEQEISFEEIKAFDDKGY |
| | | LLQKPSPNKSIYCYQSVSPKPFITSKYHNV |
| | | NLPEEYIGYYRKSNEPIVSPYQFDRLRIPI |
| | | GEPGYVPKWQYTFLSKKENKRRKLSKRIKN |
| | | VSPILGIICIKKDWCVFDMRGLLRTNHWKK |
| | | YHKPTDSINDLFDYFTGDPVIDTKANVVRF |
| | | RYKMENGIVNYKPVREKKGKELLENICDQN |
| | | GSCKLATVDVGQNNPVAIGLFELKKVNGEL |
| | | TKTLISRHPTPIDFCNKITAYRERYDKLES |
| | | SIKLDAIKQLTSEQKIEVDNYNNNFTPQNT |
| | | KQIVCSKLNINPNDLPWDKMISGTHFISEK |
| | | AQVSNKSEIYFTSTDKGKTKDVMKSDYKWF |
| | | QDYKPKLSKEVRDALSDIEWRLRRESLEFN |
| | | KLSKSREQDARQLANWISSMCDVIGIENLV |
| | | KKNNFFGGSGKREPGWDNFYKPKKENRWWI |
| | | NAIHKALTELSQNKGKRVILLPAMRTSITC |
| | | PKCKYCDSKNRNGEKFNCLKCGIELNADID |
| | | VATENLATVAITAQSMPKPTCERSGDAKKP |
| | | VRARKAKAPEFHDKLAPSYTVVLREAV |
| CasΦ.13 | SEQ ID | MRQPAEKTAFQVFRQEVIGTQKLSGGDAKT |
| | NO: 151 | AGRLYKQGKMEAAREWLLKGARDDVPPNFQ |
| | | PPAKCLVVAVSHPFEEWDISKTNHDVQAYI |
| | | YAQPLQAEGHLNGLSEKWEDTSADQHKLWF |
| | | EKTGVPDRGLPVQAINKIAKAAVNRAFGVV |
| | | RKVENRNEKRRSRDNRIAEHNRENGLTEVV |
| | | REAPEVATNADGFLLHPPGIDPSILSYASV |
| | | SPVPYNSSKHSFVRLPEEYQAYNVEPDAPI |
| | | PQFVVEDRFAIPPGQPGYVPEWQRLKCSTN |
| | | KHRRMRQWSNQDYKPKAGRRAKPLEFQAHL |
| | | TRERAKGALLVVMRIKEDWVVFDVRGLLRN |
| | | VEWRKVLSEEAREKLTLKGLLDLFTGDPVI |
| | | DTKRGIVTFLYKAEITKILSKRTVKTKNAR |
| | | DLLLRLTEPGEDGLRREVGLVAVDLGQTHP |
| | | IAAAIYRIGRTSAGALESTVLHRQGLREDQ |
| | | KEKLKEYRKRHTALDSRLRKEAFETLSVEQ |
| | | QKEIVTVSGSGAQITKDKVCNYLGVDPSTL |
| | | PWEKMGSYTHFISDDFLRRGGDPNIVHFDR |
| | | QPKKGKVSKKSQRIKRSDSQWVGRMRPRLS |
| | | QETAKARMEADWAAQNENEEYKRLARSKQE |
| | | LARWCVNTLLQNTRCITQCDEIVVVIEDLN |
| | | VKSLHGKGAREPGWDNFFTPKTENRWFIQI |
| | | LHKTFSELPKHRGEHVIEGCPLRTSITCPA |
| | | CSYCDKNSRNGEKFVCVACGATFHADFEVA |
| | | TYNLVRLATTGMPMPKSLERQGGGEKAGGA |
| | | RKARKKAKQVEKIVVQANANVTMNGASLHS |
| | | P |
| CasΦ.14 | SEQ ID | MSSLPTPLELLKQKHADLFKGLQFSSKDNK |
| | NO: 152 | MAGKVLKKDGEEAALAFLSERGVSRGELPN |
| | | FRPPAKTLVVAQSRPFEEFPIYRVSEAIQL |
| | | YVYSLSVKELETVPSGSSTKKEHQRFFQDS |
| | | SVPDFGYTSVQGLNKIFGLARGIYLGVITR |
| | | GENQLQKAKSKHEALNKKRRASGEAETEFD |
| | | PTPYEYMTPERKLAKPPGVNHSIMCYVDIS |
| | | VDEFDFRNPDGIVLPSEYAGYCREINTAIE |
| | | KGTVDRLGHLKGGPGYIPGHQRKESTTEGP |
| | | KINFRKGRIRRSYTALYAKRDSRRVRQGKL |
| | | ALPSYRHHMMRLNSNAESAILAVIFFGKDW |
| | | VVFDLRGLLRNVRWRNLFVDGSTPSTLLGM |
| | | FGDPVIDPKRGVVAFCYKEQIVPVVSKSIT |
| | | KMVKAPELLNKLYLKSEDPLVLVAIDLGQT |
| | | NPVGVGVYRVMNASLDYEVVTRFALESELL |
| | | REIESYRQRTNAFEAQIRAETFDAMTSEEQ |
| | | EEITRVRAFSASKAKENVCHRFGMPVDAVD |
| | | WATMGSNTIHIAKWVMRHGDPSLVEVLEYR |
| | | KDNEIKLDKNGVPKKVKLTDKRIANLTSIR |
| | | LRFSQETSKHYNDTMWELRRKHPVYQKLSK |
| | | SKADFSRRVVNSIIRRVNHLVPRARIVFII |
| | | EDLKNLGKVFHGSGKRELGWDSYFEPKSEN |
| | | RWFIQVLHKAFSETGKHKGYYIIECWPNWT |
| | | SCTCPKCSCCDSENRHGEVFRCLACGYTCN |
| | | TDFGTAPDNLVKIATTGKGLPGPKKRCKGS |
| | | SKGKNPKIARSSETGVSVTESGAPKVKKSS |
| | | PTQTSQSSSQSAP |
| CasΦ.15 | SEQ ID | MIKPTVSQFLTPGFKLIRNHSRTAGLKLKN |
| | NO: 153 | EGEEACKKFVRENEIPKDECPNFQGGPAIA |
| | | NIIAKSREFTEWEIYQSSLAIQEVIFTLPK |
| | | DKLPEPILKEEWRAQWLSEHGLDTVPYKEA |
| | | AGLNLIIKNAVNTYKGVQVKVDNKNKNNLA |
| | | KINRKNEIAKLNGEQEISFEEIKAFDDKGY |
| | | LLQKPSPNKSIYCYQSVSPKPFITSKYHNV |
| | | NLPEEYIGYYRKSNEPIVSPYQFDRLRIPI |
| | | GEPGYVPKWQYTFLSKKENKRRKLSKRIKN |
| | | VSPILGIICIKKDWCVFDMRGLLRTNHWKK |
| | | YHKPTDSINDLFDYFTGDPVIDTKANVVRF |
| | | RYKMENGIVNYKPVREKKGKELLENICDQN |
| | | GSCKLATVDVGQNNPVAIGLFELKKVNGEL |
| | | TKTLISRHPTPIDFCNKITAYRERYDKLES |
| | | SIKLDAIKQLTSEQKIEVDNYNNNFTPQNT |
| | | KQIVCSKLNINPNDLPWDKMISGTHFISEK |
| | | AQVSNKSEIYFTSTDKGKTKDVMKSDYKWF |
| | | QDYKPKLSKEVRDALSDIEWRLRRESLEFN |
| | | KLSKSREQDARQLANWISSMCDVIGIENLV |
| | | KKNNFFGGSGKREPGWDNFYKPKKENRWWI |
| | | NAIHKALTELSQNKGKRVILLPAMRTSITC |
| | | PKCKYCDSKNRNGEKFNCLKCGIELNADID |
| | | VATENLATVAITAQSMPKPTCERSGDAKKP |
| | | VRARKAKAPEFHDKLAPSYTVVLREAV |
| CasΦ.16 | SEQ ID | MSNKTTPPSPLSLLLRAHFPGLKFESQDYK |
| | NO: 154 | IAGKKLRDGGPEAVISYLTGKGQAKLKDVK |
| | | PPAKAFVIAQSRPFIEWDLVRVSRQIQEKI |
| | | FGIPATKGRPKQDGLSETAFNEAVASLEVD |
| | | GKSKLNEETRAAFYEVLGLDAPSLHAQAQN |
| | | ALIKSAISIREGVLKKVENRNEKNLSKTKR |
| | | RKEAGEEATFVEEKAHDERGYLIHPPGVNQ |
| | | TIPGYQAVVIKSCPSDFIGLPSGCLAKESA |
| | | EALTDYLPHDRMTIPKGQPGYVPEWQHPLL |
| | | NRRKNRRRRDWYSASLNKPKATCSKRSGTP |
| | | NRKNSRTDQIQSGRFKGAIPVLMRFQDEWV |
| | | IIDIRGLLRNARYRKLLKEKSTIPDLLSLF |
| | | TGDPSIDMRQGVCTFIYKAGQACSAKMVKT |
| | | KNAPEILSELTKSGPVVLVSIDLGQTNPIA |
| | | AKVSRVTQLSDGQLSHETLLRELLSNDSSD |
| | | GKEIARYRVASDRLRDKLANLAVERLSPEH |
| | | KSEILRAKNDTPALCKARVCAALGLNPEMI |
| | | AWDKMTPYTEFLATAYLEKGGDRKVATLKP |
| | | KNRPEMLRRDIKFKGTEGVRIEVSPEAAEA |
| | | YREAQWDLQRTSPEYLRLSTWKQELTKRIL |
| | | NQLRHKAAKSSQCEVVVMAFEDLNIKMMHG |
| | | NGKWADGGWDAFFIKKRENRWFMQAFHKSL |
| | | TELGAHKGVPTIEVTPHRTSITCTKCGHCD |
| | | KANRDGERFACQKCGFVAHADLEIATDNIE |
| | | RVALTGKPMPKPESERSGDAKKSVGARKAA |
| | | FKPEEDAEAAE |
| CasΦ.17 | SEQ ID | MYSLEMADLKSEPSLLAKLLRDRFPGKYWL |
| | NO: 155 | PKYWKLAEKKRLTGGEEAACEYMADKQLDS |
| | | PPPNFRPPARCVILAKSRPFEDWPVHRVAS |
| | | KAQSFVIGLSEQGFAALRAAPPSTADARRD |
| | | WLRSHGASEDDLMALEAQLLETIMGNAISL |
| | | HGGVLKKIDNANVKAAKRLSGRNEARLNKG |
| | | LQELPPEQEGSAYGADGLLVNPPGLNLNIY |
| | | CRKSCCPKPVKNTARFVGHYPGYLRDSDSI |
| | | LISGTMDRLTIIEGMPGHIPAWQREQGLVK |
| | | PGGRRRRLSGSESNMRQKVDPSTGPRRSTR |
| | | SGTVNRSNQRTGRNGDPLLVEIRMKEDWVL |
| | | LDARGLLRNLRWRESKRGLSCDHEDLSLSG |
| | | LLALFSGDPVIDPVRNEVVFLYGEGIIPVR |
| | | STKPVGTRQSKKLLERQASMGPLTLISCDL |
| | | GQTNLIAGRASAISLTHGSLGVRSSVRIEL |
| | | DPEIIKSFERLRKDADRLETEILTAAKETL |
| | | SDEQRGEVNSHEKDSPQTAKASLCRELGLH |
| | | PPSLPWGQMGPSTTFIADMLISHGRDDDAF |
| | | LSHGEFPTLEKRKKFDKRFCLESRPLLSSE |
| | | TRKALNESLWEVKRTSSEYARLSQRKKEMA |
| | | RRAVNFVVEISRRKTGLSNVIVNIEDLNVR |
| | | IFHGGGKQAPGWDGFFRPKSENRWFIQAIH |
| | | KAFSDLAAHHGIPVIESDPQRTSMTCPECG |
| | | HCDSKNRNGVRFLCKGCGASMDADFDAACR |
| | | NLERVALTGKPMPKPSTSCERLLSATTGKV |
| | | CSDHSLSHDAIEKAS |
| CasΦ.18 | SEQ ID | MEKEITELTKIRREFPNKKFSSTDMKKAGK |
| | NO: 156 | LLKAEGPDAVRDFLNSCQEIIGDFKPPVKT |
| | | NIVSISRPFEEWPVSMVGRAIQEYYFSLTK |
| | | EELESVHPGTSSEDHKSFFNITGLSNYNYT |
| | | SVQGLNLIFKNAKAIYDGTLVKANNKNKKL |
| | | EKKFNEINHKRSLEGLPIITPDFEEPFDEN |
| | | GHLNNPPGINRNIYGYQGCAAKVFVPSKHK |
| | | MVSLPKEYEGYNRDPNLSLAGFRNRLEIPE |
| | | GEPGHVPWFQRMDIPEGQIGHVNKIQRFNF |
| | | VHGKNSGKVKFSDKTGRVKRYHHSKYKDAT |
| | | KPYKFLEESKKVSALDSILAIITIGDDWVV |
| | | FDIRGLYRNVFYRELAQKGLTAVQLLDLFT |
| | | GDPVIDPKKGVVTFSYKEGVVPVFSQKIVP |
| | | RFKSRDTLEKLTSQGPVALLSVDLGQNEPV |
| | | AARVCSLKNINDKITLDNSCRISFLDDYKK |
| | | QIKDYRDSLDELEIKIRLEAINSLETNQQV |
| | | EIRDLDVFSADRAKANTVDMFDIDPNLISW |
| | | DSMSDARVSTQISDLYLKNGGDESRVYFEI |
| | | NNKRIKRSDYNISQLVRPKLSDSTRKNLND |
| | | SIWKLKRTSEEYLKLSKRKLELSRAVVNYT |
| | | IRQSKLLSGINDIVIILEDLDVKKKFNGRG |
| | | IRDIGWDNFFSSRKENRWFIPAFHKTFSEL |
| | | SSNRGLCVIEVNPAWTSATCPDCGFCSKEN |
| | | RDGINFTCRKCGVSYHADIDVATLNIARVA |
| | | VLGKPMSGPADRERLGDTKKPRVARSRKTM |
| | | KRKDISNSTVEAMVTA |
| CasΦ.19 | SEQ ID | MLVRTSTLVQDNKNSRSASRAFLKKPKMPK |
| | NO: 157 | NKHIKEPTELAKLIRELFPGQRFTRAINTQ |
| | | AGKILKHKGRDEVVEFLKNKGIDKEQFMDF |
| | | RPPTKARIVATSGAIEEFSYLRVSMAIQEC |
| | | CFGKYKFPKEKVNGKLVLETVGLTKEELDD |
| | | FLPKKYYENKKSRDRFFLKTGICDYGYTYA |
| | | QGLNEIFRNTRAIYEGVFTKVNNRNEKRRE |
| | | KKDKYNEERRSKGLSEEPYDEDESATDESG |
| | | HLINPPGVNLNIWTCEGFCKGPYVTKLSGT |
| | | PGYEVILPKVFDGYNRDPNEIISCGITDRF |
| | | AIPEGEPGHIPWHQRLEIPEGQPGYVPGHQ |
| | | RFADTGQNNSGKANPNKKGRMRKYYGHGTK |
| | | YTQPGEYQEVFRKGHREGNKRRYWEEDFRS |
| | | EAHDCILYVIHIGDDWVVCDLRGPLRDAYR |
| | | RGLVPKEGITTQELCNLFSGDPVIDPKHGV |
| | | VTFCYKNGLVRAQKTISAGKKSRELLGALT |
| | | SQGPIALIGVDLGQTEPVGARAFIVNQARG |
| | | SLSLPTLKGSFLLTAENSSSWNVFKGEIKA |
| | | YREAIDDLAIRLKKEAVATLSVEQQTEIES |
| | | YEAFSAEDAKQLACEKFGVDSSFILWEDMT |
| | | PYHTGPATYYFAKQFLKKNGGNKSLIEYIP |
| | | YQKKKSKKTPKAVLRSDYNIACCVRPKLLP |
| | | ETRKALNEAIRIVQKNSDEYQRLSKRKLEF |
| | | CRRVVNYLVRKAKKLTGLERVIIAIEDLKS |
| | | LEKFFTGSGKRDNGWSNFFRPKKENRWFIP |
| | | AFHKAFSELAPNRGFYVIECNPARTSITDP |
| | | DCGYCDGDNRDGIKFECKKCGAKHHTDLDV |
| | | APLNIAIVAVTGRPMPKTVSNKSKRERSGG |
| | | EKSVGASRKRNHRKSKANQEMLDATSSAAE |
| CasΦ.20 | SEQ ID | MPKIKKPTEISLLRKEVFPDLHFAKDRMRA |
| | NO: 158 | ASLVLKNEGREAAIEYLRVNHEDKPPNFMP |
| | | PAKTPYVALSRPLEQWPIAQASIAIQKYIF |
| | | GLTKDEFSATKKLLYGDKSTPNTESRKRWF |
| | | EVTGVPNFGYMSAQGLNAIFSGALARYEGV |
| | | VQKVENRNKKRFEKLSEKNQLLIEEGQPVK |
| | | DYVPDTAYHTPETLQKLAENNHVRVEDLGD |
| | | MIDRLVHPPGIHRSIYGYQQVPPFAYDPDN |
| | | PKGIILPKAYAGYTRKPHDIIEAMPNRLNI |
| | | PEGQAGYIPEHQRDKLKKGGRVKRLRTTRV |
| | | RVDATETVRAKAEALNAEKARLRGKEAILA |
| | | VFQIEEDWALIDMRGLLRNVYMRKLIAAGE |
| | | LTPTTLLGYFTETLTLDPRRTEATFCYHLR |
| | | SEGALHAEYVRHGKNTRELLLDLTKDNEKI |
| | | ALVTIDLGQRNPLAAAIFRVGRDASGDLTE |
| | | NSLEPVSRMLLPQAYLDQIKAYRDAYDSFR |
| | | QNIWDTALASLTPEQQRQILAYEAYTPDDS |
| | | KENVLRLLLGGNVMPDDLPWEDMTKNTHYI |
| | | SDRYLADGGDPSKVWFVPGPRKRKKNAPPL |
| | | KKPPKPRELVKRSDHNISHLSEFRPQLLKE |
| | | TRDAFEKAKIDTERGHVGYQKLSTRKDQLC |
| | | KEILNWLEAEAVRLTRCKTMVLGLEDLNGP |
| | | FFNQGKGKVRGWVSFFRQKQENRWIVNGFR |
| | | KNALARAHDKGKYILELWPSWTSQTCPKCK |
| | | HVHADNRHGDDFVCLQCGARLHADAEVATW |
| | | NLAVVAIQGHSLPGPVREKSNDRKKSGSAR |
| | | KSKKANESGKVVGAWAAQATPKRATSKKET |
| | | GTARNPVYNPLETQASCPAP |
| CasΦ.21 | SEQ ID | MTPSPQIARLVETPLAAALKAHHPGKKFRS |
| | NO: 159 | DYLKKAGKILKDQGVEAAMAHLDGKDQAEP |
| | | PNFKPPAKCRIVARSREFSEWPIVKASVEI |
| | | QKYIYGLTLEERKACDPGKSSASHKAWFAK |
| | | TGVNTFGYSSVQGFNLIFGHTLGRYDGVLV |
| | | KTENLNKKRAEKNERFRAKALAEGRAEPVC |
| | | PPLVTATNDTGQDVTLEDGRVVRPGQLLQP |
| | | PGINPNIYAYQQVSPKAYVPGIIELPEEFQ |
| | | GYSRDPNAVILPLVPRDRLSIPKGQPGYVP |
| | | EPHREGLTGRKDRRMRRYYETERGTKLKRP |
| | | PLTAKGRADKANEALLVVVRIDSDWVVMDV |
| | | RGLLRNARWRRLVSKEGITLNGLLDLFTGD |
| | | PVLNPKDCSVSRDTGDPVNDPRHGVVTFCY |
| | | KLGVVDVCSKDRPIKGFRTKEVLERLTSSG |
| | | TVGMVSIDLGQTNPVAAAVSRVTKGLQAET |
| | | LETFTLPDDLLGKVRAYRAKTDRMEEGFRR |
| | | NALRKLTAEQQAEITRYNDATEQQAKALVC |
| | | STYGIGPEEVPWERMTSNTTYISDHILDHG |
| | | GDPDTVFFMATKRGQNKPTLHKRKDKAWGQ |
| | | KFRPAISVETRLARQAAEWELRRASLEFQK |
| | | LSVWKTELCRQAVNYVMERTKKRTQCDVII |
| | | PVIEDLPVPLFHGSGKRDPGWANFFVHKRE |
| | | NRWFIDGLHKAFSELGKHRGIYVFEVCPQR |
| | | TSITCPKCGHCDPDNRDGEKFVCLSCQATL |
| | | NADLDVATTNLVRVALTGKVMPRSERSGDA |
| | | QTPGPARKARTGKIKGSKPTSAPQGATQTD |
| | | AKAHLSQTGV |
| CasΦ.22 | SEQ ID | MTPSPQIARLVETPLAAALKAHHPGKKFRS |
| | NO: 160 | DYLKKAGKILKDQGVEAAMAHLDGKDQAEP |
| | | PNFKPPAKCRIVARSREFSEWPIVKASVEI |
| | | QKYIYGLTLEERKACDPGKSSASHKAWFAK |
| | | TGVNTFGYSSVQGFNLIFGHTLGRYDGVLV |
| | | KTENLNKKRAEKNERFRAKALAEGRAEPVC |
| | | PPLVTATNDTGQDVTLEDGRVVRPGQLLQP |
| | | PGINPNIYAYQQVSPKAYVPGIIELPEEFQ |
| | | GYSRDPNAVILPLVPRDRLSIPKGQPGYVP |
| | | EPHREGLTGRKDRRMRRYYETERGTKLKRP |
| | | PLTAKGRADKANEALLVVVRIDSDWVVMDV |
| | | RGLLRNARWRRLVSKEGITLNGLLDLFTGD |
| | | PVLNPKDCSVSRDTGDPVNDPRHGVVTFCY |
| | | KLGVVDVCSKDRPIKGFRTKEVLERLTSSG |
| | | TVGMVSIDLGQTNPVAAAVSRVTKGLQAET |
| | | LETFTLPDDLLGKVRAYRAKTDRMEEGFRR |
| | | NALRKLTAEQQAEITRYNDATEQQAKALVC |
| | | STYGIGPEEVPWERMTSNTTYISDHILDHG |
| | | GDPDTVFFMATKRGQNKPTLHKRKDKAWGQ |
| | | KFRPAISVETRLARQAAEWELRRASLEFQK |
| | | LSVWKTELCRQAVNYVMERTKKRTQCDVII |
| | | PVIEDLPVPLFHGSGKRDPGWANFFVHKRE |
| | | NRWFIDGLHKAFSELGKHRGIYVFEVCPQR |
| | | TSITCPKCGHCDPDNRDGEKFVCLSCQATL |
| | | HADLDVATTNLVRVALTGKVMPRSERSGDA |
| | | QTPGPARKARTGKIKGSKPTSAPQGATQTD |
| | | AKAHLSQTGV |
| CasΦ.23 | SEQ ID | MKTEKPKTALTLLREEVFPGKKYRLDVLKE |
| | NO: 161 | AGKKLSTKGREATIEFLTGKDEERPQNFQP |
| | | PAKTSIVAQSRPFDQWPIVQVSLAVQKYIY |
| | | GLTQSEFEANKKALYGETGKAISTESRRAW |
| | | FEATGVDNFGFTAAQGINPIFSQAVARYEG |
| | | VIKKVENRNEKKLKKLTKKNLLRLESGEEI |
| | | EDFEPEATFNEEGRLLQPPGANPNIYCYQQ |
| | | ISPRIYDPSDPKGVILPQIYAGYDRKPEDI |
| | | ISAGVPNRLAIPEGQPGYIPEHQRAGLKTQ |
| | | GRIRCRASVEAKARAAILAVVHLGEDWVVL |
| | | DLRGLLRNVYWRKLASPGTLTLKGLLDFFT |
| | | GGPVLDARRGIATFSYTLKSAAAVHAENTY |
| | | KGKGTREVLLKLTENNSVALVTVDLGQRNP |
| | | LAAMIARVSRTSQGDLTYPESVEPLTRLFL |
| | | PDPFLEEVRKYRSSYDALRLSIREAAIASL |
| | | TPEQQAEIRYIEKFSAGDAKKNVAEVFGID |
| | | PTQLPWDAMTPRTTYISDLFLRMGGDRSRV |
| | | FFEVPPKKAKKAPKKPPKKPAGPRIVKRTD |
| | | GMIARLREIRPRLSAETNKAFQEARWEGER |
| | | SNVAFQKLSVRRKQFARTVVNHLVQTAQKM |
| | | SRCDTVVLGIEDLNVPFFHGRGKYQPGWEG |
| | | FFRQKKENRWLINDMHKALSERGPHRGGYV |
| | | LELTPFWTSLRCPKCGHTDSANRDGDDFVC |
| | | VKCGAKLHSDLEVATANLALVAITGQSIPR |
| | | PPREQSSGKKSTGTARMKKTSGETQGKGSK |
| | | ACVSEALNKIEQGTARDPVYNPLNSQVSCP |
| | | AP |
| Cas.24 | SEQ ID | VYNPDMKKPNNIRRIREEHFEGLCFGKDVL |
| | NO: 162 | TKAGKIYEKDGEEAAIDFLMGKDEEDPPNF |
| | | KPPAKTTIVAQSRPFDQWPIYQVSQAVQER |
| | | VFAYTEEEFNASKEALFSGDISSKSRDFWF |
| | | KTNNISDQGIGAQGLNTILSHAFSRYSGVI |
| | | KKVENRNKKRLKKLSKKNQLKIEEGLEILE |
| | | FKPDSAFNENGLLAQPPGINPNIYGYQAVT |
| | | PFVFDPDNPGDVILPKQYEGYSRKPDDIIE |
| | | KGPSRLDIPKGQPGYVPEHQRKNLKKKGRV |
| | | RLYRRTPPKTKALASILAVLQIGKDWVLFD |
| | | MRGLLRSVYMREAATPGQISAKDLLDTFTG |
| | | CPVLNTRTGEFTFCYKLRSEGALHARKIYT |
| | | KGETRTLLTSLTSENNTIALVTVDLGQRNP |
| | | AAIMISRLSRKEELSEKDIQPVSRRLLPDR |
| | | YLNELKRYRDAYDAFRQEVRDEAFTSLCPE |
| | | HQEQVQQYEALTPEKAKNLVLKHFFGTHDP |
| | | DLPWDDMTSNTHYIANLYLERGGDPSKVFF |
| | | TRPLKKDSKSKKPRKPTKRTDASISRLPEI |
| | | RPKMPEDARKAFEKAKWEIYTGHEKFPKLA |
| | | KRVNQLCREIANWIEKEAKRLTLCDTVVVG |
| | | IEDLSLPPKRGKGKFQETWQGFFRQKFENR |
| | | WVIDTLKKAIQNRAHDKGKYVLGLAPYWTS |
| | | QRCPACGFIHKSNRNGDHFKCLKCEALFHA |
| | | DSEVATWNLALVAVLGKGITNPDSKKPSGQ |
| | | KKTGTTRKKQIKGKNKGKETVNVPPTTQEV |
| | | EDIIAFFEKDDETVRNPVYKPTGT |
| CasΦ.25 | SEQ ID | MKKPNNIRRIREEHFEGLCFGKDVLTKAGK |
| | NO: 163 | IYEKDGEEAAIDFLMGKDEEDPPNFKPPAK |
| | | TTIVAQSRPFDQWPIYQVSQAVQERVFAYT |
| | | EEEFNASKEALFSGDISSKSRDFWFKTNNI |
| | | SDQGIGAQGLNTILSHAFSRYSGVIKKVEN |
| | | RNKKRLKKLSKKNQLKIEEGLEILEFKPDS |
| | | AFNENGLLAQPPGINPNIYGYQAVTPFVFD |
| | | PDNPGDVILPKQYEGYSRKPDDIIEKGPSR |
| | | LDIPKGQPGYVPEHQRKNLKKKGRVRLYRR |
| | | TPPKTKALASILAVLQIGKDWVLFDMRGLL |
| | | RSVYMREAATPGQISAKDLLDTFTGCPVLN |
| | | TRTGEFTFCYKLRSEGALHARKIYTKGETR |
| | | TLLTSLTSENNTIALVTVDLGQRNPAAIMI |
| | | SRLSRKEELSEKDIQPVSRRLLPDRYLNEL |
| | | KRYRDAYDAFRQEVRDEAFTSLCPEHQEQV |
| | | QQYEALTPEKAKNLVLKHFFGTHDPDLPWD |
| | | DMTSNTHYIANLYLERGGDPSKVFFTRPLK |
| | | KDSKSKKPRKPTKRTDASISRLPEIRPKMP |
| | | EDARKAFEKAKWEIYTGHEKFPKLAKRVNQ |
| | | LCREIANWIEKEAKRLTLCDTVVVGIEDLS |
| | | LPPKRGKGKFQETWQGFFRQKFENRWVIDT |
| | | LKKAIQNRAHDKGKYVLGLAPYWTSQRCPA |
| | | CGFIHKSNRNGDHFKCLKCEALFHADSEVA |
| | | TWNLALVAVLGKGITNPDSKKPSGQKKTGT |
| | | TRKKQIKGKNKGKETVNVPPTTQEVEDIIA |
| | | FFEKDDETVRNPVYKPTGT |
| Cas.26 | SEQ ID | VIKTHFPAGRFRKDHQKTAGKKLKHEGEEA |
| | NO: 164 | CVEYLRNKVSDYPPNFKPPAKGTIVAQSRP |
| | | FSEWPIVRASEAIQKYVYGLTVAELDVFSP |
| | | GTSKPSHAEWFAKTGVENYGYRQVQGLNTI |
| | | FQNTVNRFKGVLKKVENRNKKSLKRQEGAN |
| | | RRRVEEGLPEVPVTVESATDDEGRLLQPPG |
| | | VNPSIYGYQGVAPRVCTDLQGFSGMSVDFA |
| | | GYRRDPDAVLVESLPEGRLSIPKGERGYVP |
| | | EWQRDPERNKFPLREGSRRQRKWYSNACHK |
| | | PKPGRTSKYDPEALKKASAKDALLVSISIG |
| | | EDWAIIDVRGLLRDARRRGFTPEEGLSLNS |
| | | LLGLFTEYPVFDVQRGLITFTYKLGQVDVH |
| | | SRKTVPTFRSRALLESLVAKEEIALVSVDL |
| | | GQTNPASMKVSRVRAQEGALVAEPVHRMFL |
| | | SDVLLGELSSYRKRMDAFEDAIRAQAFETM |
| | | TPEQQAEITRVCDVSVEVARRRVCEKYSIS |
| | | PQDVPWGEMTGHSTFIVDAVLRKGGDESLV |
| | | YFKNKEGETLKFRDLRISRMEGVRPRLTKD |
| | | TRDALNKAVLDLKRAHPTFAKLAKQKLELA |
| | | RRCVNFIEREAKRYTQCERVVFVIEDLNVG |
| | | FFHGKGKRDRGWDAFFTAKKENRWVIQALH |
| | | KAFSDLGLHRGSYVIEVTPQRTSMTCPRCG |
| | | HCDKGNRNGEKFVCLQCGATLHADLEVATD |
| | | NIERVALTGKAMPKPPVRERSGDVQKAGTA |
| | | RKARKPLKPKQKTEPSVQEGSSDDGVDKSP |
| | | GDASRNPVYNPSDTLSI |
| CasΦ.27 | SEQ ID | MAKAKTLAALLRELLPGQHLAPHHRWVANK |
| | NO: 165 | LLMTSGDAAAFVIGKSVSDPVRGSFRKDVI |
| | | TKAGRIFKKDGPDAAAAFLDGKWEDRPPNF |
| | | QPPAKAAIVAISRSFDEWPIVKVSCAIQQY |
| | | LYALPVQEFESSVPEARAQAHAAWFQDTGV |
| | | DDCNFKSTQGLNAIFNHGKRTYEGVLKKAQ |
| | | NRNDKKNLRLERINAKRAEAGQAPLVAGPD |
| | | ESPTDDAGCLLHPPGINANIYCYQQVSPRP |
| | | YEQSCGIQLPPEYAGYNRLSNVAIPPMPNR |
| | | LDIPQGQPGYVPEHHRHGIKKFGRVRKRYG |
| | | VVPGRNRDADGKRTRQVLTEAGAAAKARDS |
| | | VLAVIRIGDDWTVVDLRGLLRNAQWRKLVP |
| | | DGGITVQGLLDLFTGDPVIDPRRGVVTFIY |
| | | KADSVGIHSEKVCRGKQSKNLLERLCAMPE |
| | | KSSTRLDCARQAVALVSVDLGQRNPVAARF |
| | | SRVSLAEGQLQAQLVSAQFLDDAMVAMIRS |
| | | YREEYDRFESLVREQAKAALSPEQLSEIVR |
| | | HEADSAESVKSCVCAKFGIDPAGLSWDKMT |
| | | SGTWRIADHVQAAGGDVEWFFFKTCGKGKE |
| | | IKTVRRSDFNVAKQFRLRLSPETRKDWNDA |
| | | IWELKRGNPAYVSFSKRKSEFARRVVNDLV |
| | | HRARRAVRCDEVVFAIEDLNISFFHGKGQR |
| | | QMGWDAFFEVKQENRWFIQALHKAFVERAT |
| | | HKGGYVLEVAPARTSTTCPECRHCDPESRR |
| | | GEQFCCIKCRHTCHADLEVATFNIEQVALT |
| | | GVSLPKRLSSTLL |
| CasΦ.28 | SEQ ID | MSKEKTPPSAYAILKAKHFPDLDFEKKHKM |
| | NO: 166 | MAGRMFKNGASEQEVVQYLQGKGSESLMDV |
| | | KPPAKSPILAQSRPFDEWEMVRTSRLIQET |
| | | IFGIPKRGSIPKRDGLSETQFNELVASLEV |
| | | GGKPMLNKQTRAIFYGLLGIKPPTFHAMAQ |
| | | NILIDLAINIRKGVLKKVDNLNEKNRKKVK |
| | | RIRDAGEQDVMVPAEVTAHDDRGYLNHPPG |
| | | VNPTIPGYQGVVIPFPEGFEGLPSGMTPVD |
| | | WSHVLVDYLPHDRLSIPKGSPGYIPEWQRP |
| | | LLNRHKGRRHRSWYANSLNKPRKSRTEEAK |
| | | DRQNAGKRTALIEAERLKGVLPVLMRFKED |
| | | WLIIDARGLLRNARYRGVLPEGSTLGNLID |
| | | LFSDSPRVDTRRGICTFLYRKGRAYSTKPV |
| | | KRKESKETLLKLTEKSTIALVSIDLGQTNP |
| | | LTAKLSKVRQVDGCLVAEPVLRKLIDNASE |
| | | DGKEIARYRVAHDLLRARILEDAIDLLGIY |
| | | KDEVVRARSDTPDLCKERVCRFLGLDSQAI |
| | | DWDRMTPYTDFIAQAFVAKGGDPKVVTIKP |
| | | NGKPKMFRKDRSIKNMKGIRLDISKEASSA |
| | | YREAQWAIQRESPDFQRLAVWQSQLTKRIV |
| | | NQLVAWAKKCTQCDTVVLAFEDLNIGMMHG |
| | | SGKWANGGWNALFLHKQENRWFMQAFHKAL |
| | | TELSAHKGIPTIEVLPHRTSITCTQCGHCH |
| | | PGNRDGERFKCLKCEFLANTDLEIATDNIE |
| | | RVALTGLPMPKGERSSAKRKPGGTRKTKKS |
| | | KHSGNSPLAAE |
| CasΦ.29 | SEQ ID | MEKAGPTSPLSVLIHKNFEGCRFQIDHLKI |
| | NO: 167 | AGRKLAREGEAAAIEYLLDKKCEGLPPNFQ |
| | | PPAKGNVIAQSRPFTEWAPYRASVAIQKYI |
| | | YSLSVDERKVCDPGSSSDSHEKWFKQTGVQ |
| | | NYGYTHVQGLNLIFKHALARYDGVLKKVDN |
| | | RNEKNRKKAERVNSFRREEGLPEEVFEEEK |
| | | ATDETGHLLQPPGVNHSIYCYQSVRPKPFN |
| | | PRKPGGISLPEAYSGYSLKPQDELPIGSLD |
| | | RLSIPPGQPGYVPEWQRSQLTTQKHRRKRS |
| | | WYSAQKWKPRTGRTSTFDPDRLNCARAQGA |
| | | ILAVVRIHEDWVVFDVRGLLRNALWRELAG |
| | | KGLTVRDLLDFFTGDPVVDTKRGVVTFTYK |
| | | LGKVDVHSLRTVRGKRSKKVLEDLTLSSDV |
| | | GLVTIDLGQTNVLAADYSKVTRSENGELLA |
| | | VPLSKSFLPKHLLHEVTAYRTSYDQMEEGF |
| | | RRKALLTLTEDQQVEVTLVRDFSVESSKTK |
| | | LLQLGVDVTSLPWEKMSSNTTYISDQLLQQ |
| | | GADPASLFFDGERDGKPCRHKKKDRTWAYL |
| | | VRPKVSPETRKALNEALWALKNTSPEFESL |
| | | SKRKIQFSRRCMNYLLNEAKRISGCGQVVF |
| | | VIEDLNVRVHHGRGKRAIGWDNFFKPKREN |
| | | RWFMQALHKAASELAIHRGMHIIEACPARS |
| | | SITCPKCGHCDPENRCSSDREKFLCVKCGA |
| | | AFHADLEVATFNLRKVALTGTALPKSIDHS |
| | | RDGLIPKGARNRKLKEPQANDEKACA |
| CasΦ.30 | SEQ ID | MKEQSPLSSVLKSNFPGKKFLSADIRVAGR |
| | NO: 168 | KLAQLGEAAAVEYLSPRQRDSVPNFRPPAF |
| | | CTVVAKSRPFEEWPIYKASVLLQEQIYGMT |
| | | GQEFEERCGSIPTSLSGLRQWASSVGLGAA |
| | | MEGLHVQGMNLMVKNAINRYKGVLVKVENR |
| | | NKKLVEANEAKNSSREERGLPPLRPPELGS |
| | | AFGPDGRLVNPPGIDKSIRLYQGVSPVPVV |
| | | KTTGRPTVHRLDIPAGEKGHVPLWQREAGL |
| | | VKEGPRRRRMWYSNSNLKRSRKDRSAEASE |
| | | ARKADSVVVRVSVKEDWVDIDVRGLLRNVA |
| | | WRGIERAGESTEDLLSLFSGDPVVDPSRDS |
| | | VVFLYKEGVVDVLSKKVVGAGKSRKQLEKM |
| | | VSEGPVALVSCDLGQTNYVAARVSVLDESL |
| | | SPVRSFRVDPREFPSADGSQGVVGSLDRIR |
| | | ADSDRLEAKLLSEAEASLPEPVRAEIEFLR |
| | | SERPSAVAGRLCLKLGIDPRSIPWEKMGST |
| | | TSFISEALSAKGSPLALHDGAPIKDSRFAH |
| | | AARGRLSPESRKALNEALWERKSSSREYGV |
| | | ISRRKSEASRRMANAVLSESRRLTGLAVVA |
| | | VNLEDLNMVSKFFHGRGKRAPGWAGFFTPK |
| | | MENRWFIRSIHKAMCDLSKHRGITVIESRP |
| | | ERTSISCPECGHCDPENRSGERFSCKSCGV |
| | | SLHADFEVATRNLERVALTGKPMPRRENLH |
| | | SPEGATASRKTRKKPREATASTFLDLRSVL |
| | | SSAENEGSGPAARAG |
| CasΦ.31 | SEQ ID | MLPPSNKIGKSMSLKEFINKRNFKSSIIKQ |
| | NO: 169 | AGKILKKEGEEAVKKYLDDNYVEGYKKRDF |
| | | PITAKCNIVASNRKIEDFDISKFSSFIQNY |
| | | VFNLNKDNFEEFSKIKYNRKSFDELYKKIA |
| | | NEIGLEKPNYENIQGEIAVIRNAINIYNGV |
| | | LKKVENRNKKIQEKNQSKDPPKLLSAFDDN |
| | | GFLAERPGINETIYGYQSVRLRHLDVEKDK |
| | | DIIVQLPDIYQKYNKKSTDKISVKKRLNKY |
| | | NVDEYGKLISKRRKERINKDDAILCVSNFG |
| | | DDWIIFDARGLLRQTYRYKLKKKGLCIKDL |
| | | LNLFTGDPIINPTKTDLKEALSLSFKDGII |
| | | NNRTLKVKNYKKCPELISELIRDKGKVAMI |
| | | SIDLGQTNPISYRLSKFTANNVAYIENGVI |
| | | SEDDIVKMKKWREKSDKLENLIKEEAIASL |
| | | SDDEQREVRLYENDIADNTKKKILEKFNIR |
| | | EEDLDFSKMSNNTYFIRDCLKNKNIDESEF |
| | | TFEKNGKKLDPTDACFAREYKNKLSELTRK |
| | | KINEKIWEIKKNSKEYHKISIYKKETIRYI |
| | | VNKLIKQSKEKSECDDIIVNIEKLQIGGNF |
| | | FGGRGKRDPGWNNFFLPKEENRWFINACHK |
| | | AFSELAPHKGIIVIESDPAYTSQTCPKCEN |
| | | CDKENRNGEKFKCKKCNYEANADIDVATEN |
| | | LEKIAKNGRRLIKNFDQLGERLPGAEMPGG |
| | | ARKRKPSKSLPKNGRGAGVGSEPELINQSP |
| | | SQVIA |
| CasΦ.32 | SEQ ID | VPDKKETPLVALCKKSFPGLRFKKHDSRQA |
| | NO: 170 | GRILKSKGEGAAVAFLEGKGGTTQPNFKPP |
| | | VKCNIVAMSRPLEEWPIYKASVVIQKYVYA |
| | | QSYEEFKATDPGKSEAGLRAWLKATRVDTD |
| | | GYFNVQGLNLIFQNARATYEGVLKKVENRN |
| | | SKKVAKIEQRNEHRAERGLPLLTLDEPETA |
| | | LDETGHLRHRPGINCSVFGYQHMKLKPYVP |
| | | GSIPGVTGYSRDPSTPIAACGVDRLEIPEG |
| | | QPGYVPPWDRENLSVKKHRRKRASWARSRG |
| | | GAIDDNMLLAVVRVADDWALLDLRGLLRNT |
| | | QYRKLLDRSVPVTIESLLNLVTNDPTLSVV |
| | | KKPGKPVRYTATLIYKQGVVPVVKAKVVKG |
| | | SYVSKMLDDTTETFSLVGVDLGVNNLIAAN |
| | | ALRIRPGKCVERLQAFTLPEQTVEDFFRFR |
| | | KAYDKHQENLRLAAVRSLTAEQQAEVLALD |
| | | TFGPEQAKMQVCGHLGLSVDEVPWDKVNSR |
| | | SSILSDLAKERGVDDTLYMFPFFKGKGKKR |
| | | KTEIRKRWDVNWAQHFRPQLTSETRKALNE |
| | | AKWEAERNSSKYHQLSIRKKELSRHCVNYV |
| | | IRTAEKRAQCGKVIVAVEDLHHSFRRGGKG |
| | | SRKSGWGGFFAAKQEGRWLMDALFGAFCDL |
| | | AVHRGYRVIKVDPYNTSRTCPECGHCDKAN |
| | | RDRVNREAFICVCCGYRGNADIDVAAYNIA |
| | | MVAITGVSLRKAARASVASTPLESLAAE |
| Cas.33 | SEQ ID | MSKTKELNDYQEALARRLPGVRHQKSVRRA |
| | NO: 171 | ARLVYDRQGEDAMVAFLDGKEVDEPYTLQP |
| | | PAKCHILAVSRPIEEWPIARVTMAVQEHVY |
| | | ALPVHEVEKSRPETTEGSRSAWFKNSGVSN |
| | | HGVTHAQTLNAILKNAYNVYNGVIKKVENR |
| | | NAKKRDSLAAKNKSRERKGLPHFKADPPEL |
| | | ATDEQGYLLQPPSPNSSVYLVQQHLRTPQI |
| | | DLPSGYTGPVVDPRSPIPSLIPIDRLAIPP |
| | | GQPGYVPLHDREKLTSNKHRRMKLPKSLRA |
| | | QGALPVCFRVFDDWAVVDGRGLLRHAQYRR |
| | | LAPKNVSIAELLELYTGDPVIDIKRNLMTF |
| | | RFAEAVVEVTARKIVEKYHNKYLLKLTEPK |
| | | GKPVREIGLVSIDLNVQRLIALAIYRVHQT |
| | | GESQLALSPCLHREILPAKGLGDFDKYKSK |
| | | FNQLTEEILTAAVQTLTSAQQEEYQRYVEE |
| | | SSHEAKADLCLKYSITPHELAWDKMTSSTQ |
| | | YISRWLRDHGWNASDFTQITKGRKKVERLW |
| | | SDSRWAQELKPKLSNETRRKLEDAKHDLQR |
| | | ANPEWQRLAKRKQEYSRHLANTVLSMAREY |
| | | TACETVVIAIENLPMKGGFVDGNGSRESGW |
| | | DNFFTHKKENRWMIKDIHKALSDLAPNRGV |
| | | HVLEVNPQYTSQTCPECGHRDKANRDPIQR |
| | | ERFCCTHCGAQRHADLEVATHNIAMVATTG |
| | | KSLTGKSLAPQRLQEAAE |
| CasΦ.41 | SEQ ID | VLLSDRIQYTDPSAPIPAMTVVDRRKIKKG |
| | NO: 172 | EPGYVPPFMRKNLSTNKHRRMRLSRGQKEA |
| | | CALPVGLRLPDGKDGWDFIIFDGRALLRAC |
| | | RRLRLEVTSMDDVLDKFTGDPRIQLSPAGE |
| | | TIVTCMLKPQHTGVIQQKLITGKMKDRLVQ |
| | | LTAEAPIAMLTVDLGEHNLVACGAYTVGQR |
| | | RGKLQSERLEAFLLPEKVLADFEGYRRDSD |
| | | EHSETLRHEALKALSKRQQREVLDMLRTGA |
| | | DQARESLCYKYGLDLQALPWDKMSSNSTFI |
| | | AQHLMSLGFGESATHVRYRPKRKASERTIL |
| | | KYDSRFAAEEKIKLTDETRRAWNEAIWECQ |
| | | RASQEFRCLSVRKLQLARAAVNWTLTQAKQ |
| | | RSRCPRVVVVVEDLNVRFMHGGGKRQEGWA |
| | | GFFKARSEKRWFIQALHKAYTELPTNRGIH |
| | | VMEVNPARTSITCTKCGYCDPENRYGEDFH |
| | | CRNPKCKVRGGHVANADLDIATENLARVAL |
| | | SGPMPKAPKLK |
| CasΦ.34 | SEQ ID | MTPSFGYQMIIVTPIHHASGAWATLRLLFL |
| | NO: 173 | NPKTSGVMLGMTKTKSAFALMREEVFPGLL |
| | | FKSADLKMAGRKFAKEGREAAIEYLRGKDE |
| | | ERPANFKPPAKGDIIAQSRPFDQWPIVQVS |
| | | QAIQKYIFGLTKAEFDATKTLLYGEGNHPT |
| | | TESRRRWFEATGVPDFGFTSAQGLNAIFSS |
| | | ALARYEGVIQKVENRNEKRLKKLSEKNQRL |
| | | VEEGHAVEAYVPETAFHTLESLKALSEKSL |
| | | VPLDDLMDKIDRLAQPPGINPCLYGYQQVA |
| | | PYIYDPENPRGVVLPDLYLGYCRKPDDPIT |
| | | ACPNRLDIPKGQPGYIPEHQRGQLKKHGRV |
| | | RRFRYTNPQAKARAKAQTAILAVLRIDEDW |
| | | VVMDLRGLLRNVYFREVAAPGELTARTLLD |
| | | TFTGCPVLNLRSNVVTFCYDIESKGALHAE |
| | | YVRKGWATRNKLLDLTKDGQSVALLSVDLG |
| | | QRHPVAVMISRLKRDDKGDLSEKSIQVVSR |
| | | TFADQYVDKLKRYRVQYDALRKEIYDAALV |
| | | SLPPEQQAEIRAYEAFAPGDAKANVLSVMF |
| | | QGEVSPDELPWDKMNTNTHYISDLYLRRGG |
| | | DPSRVFFVPQPSTPKKNAKKPPAPRKPVKR |
| | | TDENVSHMPEFRPHLSNETREAFQKAKWTM |
| | | ERGNVRYAQLSRFLNQIVREANNWLVSEAK |
| | | KLTQCQTVVWAIEDLHVPFFHGKGKYHETW |
| | | DGFFRQKKEDRWFVNVFHKAISERAPNKGE |
| | | YVMEVAPYRTSQRCPVCGFVDADNRHGDHF |
| | | KCLRCGVELHADLEVATWNIALVAVQGHGI |
| | | AGPPREQSCGGETAGTARKGKNIKKNKGLA |
| | | DAVTVEAQDSEGGSKKDAGTARNPVYIPSE |
| | | SQVNCPAP |
| CasΦ.35 | SEQ ID | MKPKTPKPPKTPVAALIDKHFPGKRFRASY |
| | NO: 174 | LKSVGKKLKNQGEDVAVRFLTGKDEERPPN |
| | | FQPPAKSNIVAQSRPIEEWPIHKVSVAVQE |
| | | YVYGLTVAEKEACSDAGESSSSHAAWFAKT |
| | | GVENFGYTSVQGLNKIFPPTFNRFDGVIKK |
| | | VENRNEKKRQKATRINEAKRNKGQSEDPPE |
| | | AEVKATDDAGYLLQPPGINHSVYGYQSITL |
| | | CPYTAEKFPTIKLPEEYAGYHSNPDAPIPA |
| | | GVPDRLAIPEGQPGHVPEEHRAGLSTKKHR |
| | | RVRQWYAMANWKPKPKRTSKPDYDRLAKAR |
| | | AQGALLIVIRIDEDWVVVDARGLLRNVRWR |
| | | SLGKREITPNELLDLFTGDPVLDLKRGVVT |
| | | FTYAEGVVNVCSRSTTKGKQTKVLLDAMTA |
| | | PRDGKKRQIGMVAVDLGQTNPIAAEYSRVG |
| | | KNAAGTLEATPLSRSTLPDELLREIALYRK |
| | | AHDRLEAQLREEAVLKLTAEQQAENARYVE |
| | | TSEEGAKLALANLGVDTSTLPWDAMTGWST |
| | | CISDHLINHGGDTSAVFFQTIRKGTKKLET |
| | | IKRKDSSWADIVRPRLTKETREALNDFLWE |
| | | LKRSHEGYEKLSKRLEELARRAVNHVVQEV |
| | | KWLTQCQDIVIVIEDLNVRNFHGGGKRGGG |
| | | WSNFFTVKKENRWFMQALHKAFSDLAAHRG |
| | | IPVLEVYPARTSITCLGCGHCDPENRDGEA |
| | | FVCQQCGATFHADLEVATRNIARVALTGEA |
| | | MPKAPAREQPGGAKKRGTSRRRKLTEVAVK |
| | | SAEPTIHQAKNQQLNGTSRDPVYKGSELPA |
| | | L |
| CasΦ.43 | SEQ ID | MSEITDLLKANFKGKTFKSADMRMAGRILK |
| | NO: 175 | KSGAQAVIKYLSDKGAVDPPDFRPPAKCNI |
| | | IAQSRPFDEWPICKASMAIQQHIYGLTKNE |
| | | FDESSPGTSSASHEQWFAKTGVDTHGFTHV |
| | | QGLNLIFQHAKKRYEGVIKKVENYNEKERK |
| | | KFEGINERRSKEGMPLLEPRLRTAFGDDGK |
| | | FAEKPGVNPSIYLYQQTSPRPYDKTKHPYV |
| | | HAPFELKEITTIPTQDDRLKIPFGAPGHVP |
| | | EKHRSQLSMAKHKRRRAWYALSQNKPRPPK |
| | | DGSKGRRSVRDLADLKAASLADAIPLVSRV |
| | | GFDWVVIDGRGLLRNLRWRKLAHEGMTVEE |
| | | MLGFFSGDPVIDPRRNVATFIYKAEHATVK |
| | | SRKPIGGAKRAREELLKATASSDGVIRQVG |
| | | LISVDLGQTNPVAYEISRMHQANGELVAEH |
| | | LEYGLLNDEQVNSIQRYRAAWDSMNESFRQ |
| | | KAIESLSMEAQDEIMQASTGAAKRTREAVL |
| | | TMFGPNATLPWSRMSSNTTCISDALIEVGK |
| | | EEETNFVTSNGPRKRTDAQWAAYLRPRVNP |
| | | ETRALLNQAVWDLMKRSDEYERLSKRKLEM |
| | | ARQCVNFVVARAEKLTQCNNIGIVLENLVV |
| | | RNFHGSGRRESGWEGFFEPKRENRWFMQVL |
| | | HKAFSDLAQHRGVMVFEVHPAYSSQTCPAC |
| | | RYVDPKNRSSEDRERFKCLKCGRSFNADRE |
| | | VATFNIREIARTGVGLPKPDCERSRGVQTT |
| | | GTARNPGRSLKSNKNPSEPKRVLQSKTRKK |
| | | ITSTETQNEPLATDLKT |
| CasΦ.44 | SEQ ID | MTPKTESPLSALCKKHFPGKRFRTNYLKDA |
| | NO: 176 | GKILKKHGEDAVVAFLSDKQEDEPANFCPP |
| | | AKVHILAQSRPFEDWPINLASKAIQTYVYG |
| | | LTADERKTCEPGTSKESHDRWFKETGVDHH |
| | | GFTSVQGLNLIFKHTLNRYDGVIKKVETRN |
| | | EKRRSSVVRINEKKAAEGLPLIAAEAEETA |
| | | FGEDGRLLQPPGVNHSIYCFQQVSPQPYSS |
| | | KKHPQVVLPHAVQGVDPDAPIPVGRPNRLD |
| | | IPKGQPGYVPEWQRPHLSMKCKRVRMWYAR |
| | | ANWRRKPGRRSVLNEARLKEASAKGALPIV |
| | | LVIGDDWLVMDARGLLRSVFWRRVAKPGLS |
| | | LSELLNVTPTGLFSGDPVIDPKRGLVTFTS |
| | | KLGVVAVHSRKPTRGKKSKDLLLKMTKPTD |
| | | DGMPRHVGMVAIDLGQTNPVAAEYSRVVQS |
| | | DAGTLKQEPVSRGVLPDDLLKDVARYRRAY |
| | | DLTEESIRQEAIALLSEGHRAEVTKLDQTT |
| | | ANETKRLLVDRGVSESLPWEKMSSNTTYIS |
| | | DCLVALGKTDDVFFVPKAKKGKKETGIAVK |
| | | RKDHGWSKLLRPRTSPEARKALNENQWAVK |
| | | RASPEYERLSRRKLELGRRCVNHIIQETKR |
| | | WTQCEDIVVVLEDLNVGFFHGSGKRPDGWD |
| | | NFFVSKRENRWFIQVLHKAFGDLATHRGTH |
| | | VIEVHPARTSITCIKCGHCDAGNRDGESFV |
| | | CLASACGDRRHADLEVATRNVARVAITGER |
| | | MPPSEQARDVQKAGGARKRKPSARNVKSSY |
| | | PAVEPAPASP |
| CasΦ.36 | SEQ ID | MSDNKMKKLSKEEKPLTPLQILIRKYIDKS |
| | NO: 177 | QYPSGFKTTIIKQAGVRIKSVKSEQDEINL |
| | | ANWIISKYDPTYIKRDFNPSAKCQIIATSR |
| | | SVADFDIVKMSNKVQEIFFASSHLDKNVFD |
| | | IGKSKSDHDSWFERNNVDRGIYTYSNVQGM |
| | | NLIFSNTKNTYLGVAVKAQNKFSSKMKRIQ |
| | | DINNFRITNHQSPLPIPDEIKIYDDAGFLL |
| | | NPPGVNPNIFGYQSCLLKPLENKEIISKTS |
| | | FPEYSRLPADMIEVNYKISNRLKFSNDQKG |
| | | FIQFKDKLNLFKINSQELFSKRRRLSGQPI |
| | | LLVASFGDDWVVLDGRGLLRQVYYRGIAKP |
| | | GSITISELLGFFTGDPIVDPIRGVVSLGFK |
| | | PGVLSQETLKTTSARIFAEKLPNLVLNNNV |
| | | GLMSIDLGQTNPVSYRLSEITSNMSVEHIC |
| | | SDFLSQDQISSIEKAKTSLDNLEEEIAIKA |
| | | VDHLSDEDKINFANFSKLNLPEDTRQSLFE |
| | | KYPELIGSKLDFGSMGSGTSYIADELIKFE |
| | | NKDAFYPSGKKKFDLSFSRDLRKKLSDETR |
| | | KSYNDALFLEKRTNDKYLKNAKRRKQIVRT |
| | | VANSLVSKIEELGLTPVINIENLAMSGGFF |
| | | DGRGKREKGWDNFFKVKKENRWVMKDFHKA |
| | | FSELSPHHGVIVIESPPYCTSVTCTKCNFC |
| | | DKKNRNGHKFTCQRCGLDANADLDIATENL |
| | | EKVAISGKRMPGSERSSDERKVAVARKAKS |
| | | PKGKAIKGVKCTITDEPALLSANSQDCSQS |
| | | TS |
| CasΦ.37 | SEQ ID | MALSLAEVRERHFKGLRFRSSYLKRAGKIL |
| | NO: 178 | KKEGEAACVAYLTGKDEESPPNFKPPAKCD |
| | | VVAQSRPFEEWPIVQASVAVQSYVYGLTKE |
| | | AFEAFNPGTTKQSHEACLAATGIDTCGYSN |
| | | VQGLNLIFRQAKNRYEGVITKVENRNKKAK |
| | | KKLTRKNEWRQKNGHSELPEAPEELTFNDE |
| | | GRLLQPPGINPSLYTYQQISPTPWSPKDSS |
| | | ILPPQYAGYERDPNAPIPFGVAKDRLTIAS |
| | | GCPGYIPEWMRTAGEKTNPRTQKKFMHPGL |
| | | STRKNKRMRLPRSVRSAPLGALLVTIHLGE |
| | | DWLVLDVRGLLRNARWRGVAPKDISTQGLL |
| | | NLFTGDPVIDTRRGVVTFTYKPETVGIHSR |
| | | TWLYKGKQTKEVLEKLTQDQTVALVAIDLG |
| | | QTNPVSAAASRVSRSGENLSIETVDRFFLP |
| | | DELIKELRLYRMAHDRLEERIREESTLALT |
| | | EAQQAEVRALEHVVRDDAKNKVCAAFNLDA |
| | | ASLPWDQMTSNTTYLSEAILAQGVSRDQVF |
| | | FTPNPKKGSKEPVEVMRKDRAWVYAFKAKL |
| | | SEETRKAKNEALWALKRASPDYARLSKRRE |
| | | ELCRRSVNMVINRAKKRTQCQVVIPVLEDL |
| | | NIGFFHGSGKRLPGWDNFFVAKKENRWLMN |
| | | GLHKSFSDLAVHRGFYVFEVMPHRTSITCP |
| | | ACGHCDSENRDGEAFVCLSCKRTYHADLDV |
| | | ATHNLTQVAGTGLPMPEREHPGGTKKPGGS |
| | | RKPESPQTHAPILHRTDYSESADRLGS |
| CasΦ.45 | SEQ ID | QAVIKYLSDKGAVDPPDFRPPAKCNIIAQS |
| | NO: 179 | RPFDEWPICKASMAIQQHIYGLTKNEFDES |
| | | SPGTSSASHEQWFAKTGVDTHGFTHVQGLN |
| | | LIFQHAKKRYEGVIKKVENYNEKERKKFEG |
| | | INERRSKEGMPLLEPRLRTAFGDDGKFAEK |
| | | PGVNPSIYLYQQTSPRPYDKTKHPYVHAPF |
| | | ELKEITTIPTQDDRLKIPFGAPGHVPEKHR |
| | | SQLSMAKHKRRRAWYALSQNKPRPPKDGSK |
| | | GRRSVRDLADLKAASLADAIPLVSRVGFDW |
| | | VVIDGRGLLRNLRWRKLAHEGMTVEEMLGF |
| | | FSGDPVIDPRRNVATFIYKAEHATVKSRKP |
| | | IGGAKRAREELLKATASSDGVIRQVGLISV |
| | | DLGQTNPVAYEISRMHQANGELVAEHLEYG |
| | | LLNDEQVNSIQRYRAAWDSMNESFRQKAIE |
| | | SLSMEAQDEIMQASTGAAKRTREAVLTMFG |
| | | PNATLPWSRMSSNTTCISDALIEVGKEEET |
| | | NFVTSNGPRKRTDAQWAAYLRPRVNPETRA |
| | | LLNQAVWDLMKRSDEYERLSKRKLEMARQC |
| | | VNFVVARAEKLTQCNNIGIVLENLVVRNFH |
| | | GSGRRESGWEGFFEPKRENRWFMQVLHKAF |
| | | SDLAQHRGVMVFEVHPAYSSQTCPACRYVD |
| | | PKNRSSEDRERFKCLKCGRSFNADREVATF |
| | | NIREIARTGVGLPKPDCERSRDVQTPGTAR |
| | | KSGRSLKSQDNLSEPKRVLQSKTRKKITST |
| | | ETQNEPLATDLKT |
| CasΦ.38 | SEQ ID | MIKEQSELSKLIEKYYPGKKFYSNDLKQAG |
| | NO: 180 | KHLKKSEHLTAKESEELTVEFLKSCKEKLY |
| | | DFRPPAKALIISTSRPFEEWPIYKASESIQ |
| | | KYIYSLTKEELEKYNISTDKTSQENFFKES |
| | | LIDNYGFANVSGLNLIFQHTKAIYDGVLKK |
| | | VNNRNNKILKKYKRKIEEGIEIDSPELEKA |
| | | IDESGHFINPPGINKNIYCYQQVSPTIFNS |
| | | FKETKIICPFNYKRNPNDIIQKGVIDRLAI |
| | | PFGEPGYIPDHQRDKVNKHKKRIRKYYKNN |
| | | ENKNKDAILAKINIGEDWVLFDLRGLLRNA |
| | | YWRKLIPKQGITPQQLLDMFSGDPVIDPIK |
| | | NNITFIYKESIIPIHSESIIKTKKSKELLE |
| | | KLTKDEQIALVSIDLGQTNPVAARFSRLSS |
| | | DLKPEHVSSSFLPDELKNEICRYREKSDLL |
| | | EIEIKNKAIKMLSQEQQDEIKLVNDISSEE |
| | | LKNSVCKKYNIDNSKIPWDKMNGFTTFIAD |
| | | EFINNGGDKSLVYFTAKDKKSKKEKLVKLS |
| | | DKKIANSFKPKISKETREILNKITWDEKIS |
| | | SNEYKKLSKRKLEFARRATNYLINQAKKAT |
| | | RLNNVVLVVEDLNSKFFHGSGKREDGWDNF |
| | | FIPKKENRWFIQALHKSLTDVSIHRGINVI |
| | | EVRPERTSITCPKCGCCDKENRKGEDFKCI |
| | | KCDSVYHADLEVATFNIEKVAITGESMPKP |
| | | DCERLGGEESIG |
| CasΦ.39 | SEQ ID | VAFLDGKEVDEPYTLQPPAKCHILAVSRPI |
| | NO: 181 | EEWPIARVTMAVQEHVYALPVHEVEKSRPE |
| | | TTEGSRSAWFKNSGVSNHGVTHAQTLNAIL |
| | | KNAYNVYNGVIKKVENRNAKKRDSLAAKNK |
| | | SRERKGLPHFKADPPELATDEQGYLLQPPS |
| | | PNSSVYLVQQHLRTPQIDLPSGYTGPVVDP |
| | | RSPIPSLIPIDRLAIPPGQPGYVPLHDREK |
| | | LTSNKHRRMKLPKSLRAQGALPVCFRVFDD |
| | | WAVVDGRGLLRHAQYRRLAPKNVSIAELLE |
| | | LYTGDPVIDIKRNLMTFRFAEAVVEVTARK |
| | | IVEKYHNKYLLKLTEPKGKPVREIGLVSID |
| | | LNVQRLIALAIYRVHQTGESQLALSPCLHR |
| | | EILPAKGLGDFDKYKSKFNQLTEEILTAAV |
| | | QTLTSAQQEEYQRYVEESSHEAKADLCLKY |
| | | SITPHELAWDKMTSSTQYISRWLRDHGWNA |
| | | SDFTQITKGRKKVERLWSDSRWAQELKPKL |
| | | SNETRRKLEDAKHDLQRANPEWQRLAKRKQ |
| | | EYSRHLANTVLSMAREYTACETVVIAIENL |
| | | PMKGGFVDGNGSRESGWDNFFTHKKENRWM |
| | | IKDIHKALSDLAPNRGVHVLEVNPQYTSQT |
| | | CPECGHRDKANRDPIQRERFCCTHCGAQRH |
| | | ADLEVATHNIAMVATTGKSLTGKSLAPQRL |
| | | Q |
| CasΦ.42 | SEQ ID | LEIPEGEPGHVPWFQRMDIPEGQIGHVNKI |
| | NO: 182 | QRFNFVHGKNSGKVKFSDKTGRVKRYHHSK |
| | | YKDATKPYKFLEESKKVSALDSILAIITIG |
| | | DDWVVFDIRGLYRNVFYRELAQKGLTAVQL |
| | | LDLFTGDPVIDPKKGIITFSYKEGVVPVFS |
| | | QKIVSRFKSRDTLEKLTSQGPVALLSVDLG |
| | | QNEPVAARVCSLKNINDKIALDNSCRIPFL |
| | | DDYKKQIKDYRDSLDELEIKIRLEAINSLD |
| | | VNQQVEIRDLDVFSADRAKASTVDMFDIDP |
| | | NLISWDSMSDARFSTQISDLYLKNGGDESR |
| | | VYFEINNKRIKRSDYNISQLVRPKLSDSTR |
| | | KNLNDSIWKLKRTSEEYLKLSKRKLELSRA |
| | | VVNYTIRQSKLLSGINDIVIILEDLDVKKK |
| | | FNGRGIRDIGWDNFFSSRKENRWFIPAFHK |
| | | SFSELSSNRGLCVIEVNPAWTSATCPDCGF |
| | | CSKENRDGINFTCRKCGVSYHADIDVATLN |
| | | IARVAVLGKPMSGPADRERLGGTKKPRVAR |
| | | SRKDMKRKDISNGTVEVMVTA |
| CasΦ.46 | SEQ ID | IPSFGYLDRLKIAKGQPGYIPEWQRETINP |
| | NO: 183 | SKKVRRYWATNHEKIRNAIPLVVFIGDDWV |
| | | IIDGRGLLRDARRRKLADKNTTIEQLLEMV |
| | | SNDPVIDSTRGIATLSYVEGVVPVRSFIPI |
| | | GEKKGREYLEKSTQKESVTLLSVDIGQINP |
| | | VSCGVYKVSNGCSKIDFLDKFFLDKKHLDA |
| | | IQKYRTLQDSLEASIVNEALDEIDPSFKKE |
| | | YQNINSQTSNDVKKSLCTEYNIDPEAISWQ |
| | | DITAHSTLISDYLIDNNITNDVYRTVNKAK |
| | | YKTNDFGWYKKFSAKLSKEAREALNEKIWE |
| | | LKIASSKYKKLSVRKKEIARTIANDCVKRA |
| | | ETYGDNVVVAMESLTKNNKVMSGRGKRDPG |
| | | WHNLGQAKVENRWFIQAISSAFEDKATHHG |
| | | TPVLKVNPAYTSQTCPSCGHCSKDNRSSKD |
| | | RTIFVCKSCGEKFNADLDVATYNIAHVAFS |
| | | GKKLSPPSEKSSATKKPRSARKSKKSRKS |
| CasΦ.47 | SEQ ID | SPIEKLLNGLLVKITFGNDWIICDARGLLD |
| | NO: 184 | NVQKGIIHKSYFTNKSSLVDLIDLFTCNPI |
| | | VNYKNNVVTFCYKEGVVDVKSFTPIKSGPK |
| | | TQENLIKKLKYSRFQNEKDACVLGVGVDVG |
| | | VTNPFAINGFKMPVDESSEWVMLNEPLFTI |
| | | ETSQAFREEIMAYQQRTDEMNDQFNQQSID |
| | | LLPPEYKVEFDNLPEDINEVAKYNLLHTLN |
| | | IPNNFLWDKMSNTTQFISDYLIQIGRGTET |
| | | EKTITTKKGKEKILTIRDVNWFNTFKPKIS |
| | | EETGKARTEIKRDLQKNSDQFQKLAKSREQ |
| | | SCRTWVNNVTEEAKIKSGCPLIIFVIEALV |
| | | KDNRVFSGKGHRAIGWHNFGKQKNERRWWV |
| | | QAIHKAFQEQGVNHGYPVILCPPQYTSQTC |
| | | PKCNHVDRDNRSGEKFKCLKYGWIGNADLD |
| | | VGAYNIARVAITGKALSKPLEQKKIKKAKN |
| | | KT |
| CasΦ.48 | SEQ ID | LLDNVQKGIIHKSYFTNKSSLVDLIDLFTC |
| | NO: 185 | NPIVNYKNNVVTFCYKEGVVDVKSFTPIKS |
| | | GPKTQENLIKKLKYSRFQNEKDACVLGVGV |
| | | DVGVTNPFAINGFKMPVDESSEWVMLNEPL |
| | | FTIETSQAFREEIMAYQQRTDEMNDQFNQQ |
| | | SIDLLPPEYKVEFDNLPEDINEVAKYNLLH |
| | | TLNIPNNFLWDKMSNTTQFISDYLIQIGRG |
| | | TETEKTITTKKGKEKILTIRDVNWFNTFKP |
| | | KISEETGKARTEIKRDLQKNSDQFQKLAKS |
| | | REQSCRTWVNNVTEEAKIKSGCPLIIFVIE |
| | | ALVKDNRVFSGKGHRAIGWHNFGKQKNERR |
| | | WWVQAIHKAFQEQGVNHGYPVILCPPQYTS |
| | | QTCPKCNHVDRDNRSGEKFKCLKYGWIGNA |
| | | DLDVGAYNIARVAITGKALSKPLEQKKIKK |
| | | AKNKT |
| CasΦ.49 | SEQ ID | MIKPTVSQFLTPGFKLIRNHSRTAGLKLKN |
| | NO: 186 | EGEEACKKFVRENEIPKDECPNFQGGPAIA |
| | | NIIAKSREFTEWEIYQSSLAIQEVIFTLPK |
| | | DKLPEPILKEEWRAQWLSEHGLDTVPYKEA |
| | | AGLNLIIKNAVNTYKGVQVKVDNKNKNNLA |
| | | KINRKNEIAKLNGEQEISFEEIKAFDDKGY |
| | | LLQKPSPNKSIYCYQSVSPKPFITSKYHNV |
| | | NLPEEYIGYYRKSNEPIVSPYQFDRLRIPI |
| | | GEPGYVPKWQYTFLSKKENKRRKLSKRIKN |
| | | VSPILGIICIKKDWCVFDMRGLLRTNHWKK |
| | | YHKPTDSINDLFDYFTGDPVIDTKANVVRF |
| | | RYKMENGIVNYKPVREKKGKELLENICDQN |
| | | GSCKLATVDVGQNNPVAIGLFELKKVNGEL |
| | | TKTLISRHPTPIDFCNKITAYRERYDKLES |
| | | SIKLDAIKQLT |
| | | SEQKIEVDNYNNNFTPQNTKQIVCSKLNIN |
| | | PNDLPWDKMISGTHFISEKAQVSNKSEIYF |
| | | TSTDKGKTKDVMKSDYKWFQDYKPKLSKEV |
| | | RDALSDIEWRLRRESLEFNKLSKSREQDAR |
| | | QLANWISSMCDVIGIENLVKKNNFFGGSGK |
| | | REPGWDNFYKPKKENRWWINAIHKALTELS |
| | | QNKGKRVILLPAMRTSITCPKCKYCDSKNR |
| | | NGEKFNCLKCGIELNADIDVATENLATVAI |
| | | TAQSMPKPTCERSGDAKKPVRARKAKAPEF |
| | | HDKLAPSYTVVLREAVKRPAATKKAGQAKK |
| | | KKEF |
| | | (Bold sequence is Nuclear |
| | | Localization Signal) |

In some embodiments, any of the programmable Case nuclease of the present disclosure (e.g., any one of SEQ ID NO: 139-SEQ ID NO: 186 or fragments or variants thereof) can include a nuclear localization signal (NLS). In some cases, said NLS can have a sequence of KRPAATKKAGQAKKKKEF (SEQ ID NO: 187).

A Case polypeptide or a variant thereof can comprise at least 70%, at least 75%, at least 80%, at least 85%, at least 90%, at least 92%, at least 95%, at least 97%, at least 99%, or 100% sequence identity with any one of SEQ ID NO: 139-SEQ ID NO: 186.

In some embodiments, a programmable nuclease as disclosed herein is an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA reporter molecule, a catalytic oligonucleotide, or a blocker oligonucleotide, and is capable of being activated by a target DNA to initiate trans cleavage of an RNA reporter molecule, such as a Type VI CRISPR/Cas enzyme (e.g., Cas13). For example, Cas13a of the present disclosure can be activated by a target RNA to initiate trans cleavage activity of the Cas13a for the cleavage of an RNA reporter molecule, a catalytic oligonucleotide, or a blocker oligonucleotide, and can be activated by a target DNA to initiate trans cleavage activity of the Cas13a for trans cleavage of an RNA reporter molecule, a catalytic oligonucleotide, or a blocker oligonucleotide. An RNA reporter molecule can be an RNA-based reporter molecule. In some embodiments, the Cas13a recognizes and detects ssDNA to initiate transcleavage of RNA reporter molecules. Multiple Cas13a isolates can recognize, be activated by, and detect target DNA, including ssDNA, upon hybridization of a guide nucleic acid with the target DNA. For example, LbuCas13a and LwaCas13a can both be activated to transcollaterally cleave RNA reporters by target DNA. Thus, Type VI CRISPR/Cas enzyme (e.g., Cas13, such as Cas13a) can be DNA-activated programmable RNA nucleases, and therefore, can be used to detect a target DNA using the methods as described herein. DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 can exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA can not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target DNA detected by Cas13 disclosed herein can be directly from organisms, or can be indirectly generated by nucleic acid amplification methods, such as PCR and LAMP or any amplification method described herein. Key steps for the sensitive detection of a target DNA, such as a target ssDNA, by a DNA-activated programmable RNA nuclease, such as Cas13a, can include: (1) production or isolation of DNA to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection.

The detection of a target DNA by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas protein, with a DNA-activated programmable RNA nuclease, such as a Type VI protein, with a DNA reporter and an RNA reporter, can enable multiplexed detection of target ssDNAs or a combination of a target dsDNA and a target ssDNA, respectively. Multiplexing of different RNA-activated programmable RNA nucleases that have distinct RNA reporter cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein. For example target ssDNA detection by Cas13a can be employed in an assay disclosed herein.

In some embodiments, the programmable nuclease comprises a Cas12 protein, wherein the Cas12 enzyme binds and cleaves double stranded DNA and single stranded DNA. In some embodiments, programmable nuclease comprises a Cas13 protein, wherein the Cas13 enzyme binds and cleaves single stranded RNA. In some embodiments, programmable nuclease comprises a Cas14 protein, wherein the Cas14 enzyme binds and cleaves both double stranded DNA and single stranded DNA.

TABLE 5 provides illustrative amino acid sequences of programmable nucleases having trans-cleavage activity. In some instances, programmable nucleases described herein comprise an amino acid sequence that is at least 65%, at least 70%, at least 75%, at least 80%, at least 85%, at least 90%, at least 95%, at least 97%, or at least 98%, at least 99%, or 100% identical to any one of the sequences of TABLE 5.

TABLE 5
Amino Acid Sequences of Exemplary Programmable Nucleases

| SEQ ID | |
|---|---|
| NO: | Programmable Nuclease Amino Acid Sequence |
| SEQ ID | MADLSQFTHKYQVPKTLRFELIPQGKTLENLSAYGMVADDKQRSENYK |
| NO: 208 | KLKPVIDRIYKYFIEESLKNTNLDWNPLYEAIREYRKEKTTATITNLKEQ |
| | QDICRRAIASRFEGKVPDKGDKSVKDFNKKQSKLFKELFGKELFTDSVL |
| | EQLPGVSLSDEDKALLKSFDKFTTYFVGFYDNRKNVFSSDDISTGIPHRL |
| | VQENFPKFIDNCDDYKRLVLVAPELKEKLEKAAEATKIFEDVSLDEIFSIK |
| | FYNRLLQQNQIDQFNQLLGGIAGAPGTPKIQGLNETLNLSMQQDKTLEQ |
| | KLKSVPHRFSPLYKQILSDRSSLSFIPESFSCDAEVLLAVQEYLDNLKTEH |
| | VIEDLKEVFNRLTTLDLKHIYVNSTKVTAFSQALFGDWNLCREQLRVYK |
| | MSNGNEKITKKALGELESWLKNSDIAFTELQEALADEALPAKVNLKVQ |
| | EAISGLNEQMAKSLPKELKIPEEKEELKALLDAIQEVYHTLEWFIVSDDV |
| | ETDTDFYVPLKETLQIIQPIIPLYNKVRNFATQKPYSVEKFKLNFANPTLA |
| | DGWDENKEQQNCAVLFQKGNNYYLGILNPKNKPDFDNVDTEKQGNCY |
| | QKMVYKQFPDFSKMMPKCTTQLKEVKQHFEGKDSDYILNNKNFIKPLT |
| | ITREVYDLNNVLYDGKKKFQIDYLRKTKDEDGYYTALHTWIDFAKKFV |
| | ASYKSTSIYDTSTILPPEKYEKLNEFYGALDNLFYQIKFENIPEEIIDTYVE |
| | DGKLFLFQIYNKDFAAGATGAPNLHTIYWKAVFDPENVKDVVVKLNGQ |
| | AELFYRPKSNMDVIRHKVGEKLVNRTLKDGSILTDELHKELYLYANGSL |
| | KKGLSEDAKIILDKNLAVIYDVHHEIVKDRRFTTDKFFFHVPLTLNYKCD |
| | KNPVKFNAEVQEYLKENPDTYVIGIDRGERNLIYAVVIDPKGRIVEQKSF |
| | NVINGFDYHGKLDQREKERVKARQAWTAVGKIKELKQGYLSLVVHEIS |
| | KMMVRYQAVVVLENLNVGFKRVRSGIAEKAVYQQFEKMLINKLNYLM |
| | FKDAGGTEPGSVLNAYQLTDRFESFAKMGLQTGFLFYIPAAFTSKIDPAT |
| | GFVDPFRWGAIKTLADKREFLSGFESLKFDSTTGNFILHFDVSKNKNFQ |
| | KKLEGFVPDWDIIIEANKMKTGKGATYIAGKRIEFVRDNNSQGHYEDYL |
| | PCNALAETLRQCDIPYEEGKDILPLILEKNDSKLLHSVFKVVRLTLQMRN |
| | SNAETGEDYISSPVEDVSGSCFDSRMENEKLPKDADANGAYHIALKGM |
| | LALERLRKDEKMAISNNDWLNYIQEKRA* |
| SEQ ID | MAGKKKDKDVINKTLSVRIIRPRYSDDIEKEISDEKAKRKQDGKTGELD |
| NO: 209 | RAFFSELKSRNPDIITNDELFPLFTEIQKNLTEIYNKSISLLYMKLIVEEEG |
| | GSTASALSAGPYKECKARFNSYISLGLRQKIQSNFRRKELKGFQVSLPTA |
| | KSDRFPIPFCHQVENGKGGFKVYETGDDFIFEVPLIKYTATNKKSTSGKN |
| | YTKVQLNNPPVPMNVPLLLSTMRRRQTKKGMQWNKDEGTNAELRRV |
| | MSGEYKVSYAEIIRRTRFGKHDDWFVNFSIKFKNKTDELNQNVRGGIDI |
| | GVSNPLVCAVTNGLDRYIVANNDIMAFNERAMARRRTLLRKNRFKRSG |
| | HGAKNKLEPITVLTEKNERFRKSILQRWAREVAEFFKRTSASVVNMEDL |
| | SGITEREDFFSTKLRTTWNYRLMQTTIENKLKEYGIAVNYISPKYTSQTC |
| | HSCGKRNDYFTFSYRSENNYPPFECKECNKVKCNADFNAAKNIALKVV |
| | L |
| SEQ ID | MAKNTITKTLKLRIVRPYNSAEVEKIVADEKNNREKIALEKNKDKVKEA |
| NO: 210 | CSKHLKVAAYCTTQVERNACLFCKARKLDDKFYQKLRGQFPDAVFWQ |
| | EISEIFRQLQKQAAEIYNQSLIELYYEIFIKGKGIANASSVEHYLSDVCYT |
| | RAAELFKNAAIASGLRSKIKSNFRLKELKNMKSGLPTTKSDNFPIPLVKQ |
| | KGGQYTGFEISNHNSDFIIKIPFGRWQVKKEIDKYRPWEKFDFEQVQKSP |
| | KPISLLLSTQRRKRNKGWSKDEGTEAEIKKVMNGDYQTSYIEVKRGSKI |
| | GEKSAWMLNLSIDVPKIDKGVDPSIIGGIDVGVKSPLVCAINNAFSRYSIS |
| | DNDLFHFNKKMFARRRILLKKNRHKRAGHGAKNKLKPITILTEKSERFR |
| | KKLIERWACEIADFFIKNKVGTVQMENLESMKRKEDSYFNIRLRGFWPY |
| | AEMQNKIEFKLKQYGIEIRKVAPNNTSKTCSKCGHLNNYFNFEYRKKN |
| | KFPHFKCEKCNFKENADYNAALNISNPKLKSTKEEP |
| SEQ ID | MATLVSFTKQYQVQKTLRFELIPQGKTQANIDAKGFINDDLKRDENYM |
| NO: 211 | KVKGVIDELHKNFIEQTLVNVDYDWRSLATAIKNYRKDRSDTNKKNLE |
| | KTQEAARKEIIAWFEGKRGNSAFKNNQKSFYGKLFKKELFSEILRSDDL |
| | EYDEETQDAIACFDKFTTYFVGFHENRKNMYSTEAKSTSVAYRVVNEN |
| | FSKFLSNCEAFSVLEAVCPNVLVEAEQELHLHKAFSDLKLSDVFKVEAY |
| | NKYLSQTGIDYYNQIIGGISSAEGVRKIRGVNEVVNNAIQQNDELKVAL |
| | RNKQFTMVQLFKQILSDRSTLSFVSEQFTSDQEVITVVKQFNDDIVNNK |
| | VLAVVKTLFENFNSYDLEKIYINSKELASVSNALLKDWSKIRNAVLENKI |
| | IELGANPPKTKISAVEKEVKNKDFSIAELASYNDKYLDKEGNDKEICSIA |
| | NVVLEAVGALEIMLAESLPADLKTLENKNKVKGILDAYENLLHLLNYFK |
| | VSAVNDVDLAFYGAFEKVYVDISGVMPLYNKVRNYATKKPYSVEKFKL |
| | NFAMPTLADGWDKNKERDNGSIILLKDGQYYLGVMNPQNKPVIDNAV |
| | CNDAKGYQKMVYKMFPEISKMVTKCSTQLNAVKAHFEDNTNDFVLDD |
| | TDKFISDLTITKEIYDLNNVLYDGKKKFQIDYLRNTGDFAGYHKALETWI |
| | DFVKEFLSKYRSTAIYDLTTLLPTNYYEKLDVFYSDVNNLCYKIDYENIS |
| | VEQVNEWVEEGNLYLFKIYNKDFATGSTGKPNLHTMYWNAVFAEENL |
| | HDVVVKLNGGAELFYRPKSNMPKVEHRVGEKLVNRKNVNGEPIADSV |
| | HKEIYAYANGKISKSELSENAQEELPLAIIKDVKHNITKDKRYLSDKYFF |
| | HVPITLNYKANGNPSAFNTKVQAFLKNNPDVNIIGIDRGERNLLYVVVI |
| | DQQGNIIDKKQVSYNKVNGYDYYEKLNQREKERIEARQSWGAVGKIKE |
| | LKEGYLSLVVREIADMMVKYNAIVVMENLNAGFKRVRGGIAEKAVYQ |
| | KFEKMLIDKLNYLVFKDVEAKEAGGVLNAYQLTDKFDSFEKMGNQSGF |
| | LFYVPAAYTSKIDPVTGFANVFSTKHITNTEAKKEFICSFNSLRYDEAKD |
| | KFVLECDLNKFKIVANSHIKNWKFIIGGKRIVYNSKNKTYMEKYPCEDL |
| | KATLNASGIDFSSSEIINLLKNVPANREYGKLFDETYWAIMNTLQMRNS |
| | NALTGEDYIISAVADDNEKVFDSRTCGAELPKDADANGAYHIALKGLYL |
| | LQRIDISEEGEKVDLSIKNEEWFKFVQQKEYAR* |
| SEQ ID | MCMKITKIDGISHKKYKEKGKLIKNNDTAKDIIEERFNDIEKKTKELFQK |
| NO: 212 | TLDFYVKNYEKCKEQNKERREKAKNYFSKVKILVDNKKITICNENTEK |
| | MEIEDFNEYDVRSGKYFNVLNKILNGENYTEEDLEVFENDLQKRTGRIK |
| | SIKNSLEENKAHFKKESINNNIIYDRVKGNNKKSLFYEYYRISSKHQEYV |
| | NNIFEAFDKLYSNSHEAMNNLFSEITKDSKDRNIRKIREAYHEILNKNKT |
| | EFGEELYKKIQDNRNNFDKLLEIEPEIKELTKSQIFYKYYIDKVNLDETSI |
| | KHCFCHLVEIEVNQLLKNYVYSKRNINKEKLENIFEYCKLKNLIKNKLV |
| | NKLNNYIRNCGKYNAYISNNDVVVNSEKISEIRTKEAFLRSIIGVSSSAYF |
| | SLRNILNTDNTQDITNKVDKEVDKLYQENKKIELEERLKLFFGNYFDIN |
| | NQQEIKVFLMNIDKIISSIRHEIIHFKMETNAQNIFDENNVNLGNTAKNIF |
| | SNEINEEKIKFKIFKQLNSANVFDYLSNKDITEYMDKVVFSFTNRNVSFV |
| | PSFTKIYNRVQDLANSLEIKKWKIPDKSEGKDAQIYLLKNIYYGKFLDEF |
| | LNEENGIFISIKDKIIELNRNQNKRTGFYKLEKFEKIEETNPKKYLEIIQSL |
| | YMINIEEIDSEGKNIFLDFIQKIFLKGFFEFIKNNYNYLLELKKIQDKKNIF |
| | DSEMSEYIAGEKTLEDIGEINEIIQDIKITEIDKILNQTDKINCFYLLLKLL |
| | NYKEITELKGNLEKYQILSKTNVYEKELMLLNIVNLDNNKVKIENFKIL |
| | AEEIGKFIEKINIEEINKNKKIKTFEELRNFEKGENTGEYYNIYSDDKNIK |
| | NIRNLYNIKKYGMLDLLEKISEKTNYCIKKKDLEEYSELRKQLEDEKTN |
| | FYKIQEYLHSKYQQKPKKILLKNNKNDYEKYKKSIENIEKYVHLKNKIE |
| | FNELNLLQSLLLKILHRLVGFTSIWERDLRFRLIGEFPDELDVEDIFDHRK |
| | RYKGTGKGICKKYDRFINTHTEYKNNNKMENVKFADNNPVRNYIAHFN |
| | YLPNPKYSILKMMEKLRKLLDYDRKLKNAVMKSIKDILEEYGFKAEFII |
| | NSDKEIILNLVKSVEIIHLGKEDLKSRRNSEDLCKLVKAMLEYSK* |
| SEQ ID | MEDKQFLERYKEFIGLNSLSKTLRNSLIPVGSTLKHIQEYGILEEDSLRA |
| NO: 213 | QKREELKGIMDDYYRNYIEMHLRDVHDIDWNELFEALTEVKKNQTDD |
| | AKKRLEKIQEKKRKEIYQYLSDDAVFSEMFKEKMISGILPDFIRCNEGYS |
| | EEEKEEKLKTVALFHRFTSSFNDFFLNRKNVFTKEAIVTAIGYRVVHENA |
| | EIFLENMVAFQNIQKSAESQISIIERKNEHYFMEWKLSHIFTADYYMML |
| | MTQKAIEHYNEMCGVVNQQMREYCQKEKKNWNLYRMKRLHKQILSN |
| | ASTSFKIPEKYENDAEVYESVNSFLQNVMEKTVMERIAVLKNSTDNFDL |
| | SKIYITAPYYEKISNYLCGSWNTITDCLTHYYEQQIAGKGARKDQKVKA |
| | AVKADKWKSLSEIEQLLKEYARAEEVKRKPEEYIAEIENIVSLKEAHLLE |
| | YHPEVNLIENEKYATEIKDVLDNYMELFHWMKWFYIEEAVEKEVNFYG |
| | ELDDLYEEIKDIVPLYNKVRNYVTQKPYSDTKIKLNFGTPTLANGWSKS |
| | KEYDYNAILLQKDGKYYMGIFNPIQKPEKEIIEGHSQPLEGNEYKKMVY |
| | YYLPSANKMLPKVLLSKKGMEIYQPSEYIINGYKERRHIKSEEKFDLQF |
| | CHDLIDYFKSGIERNSDWKVFGFDFSDTDTYQDISGFYREVEDQGYKID |
| | WTYIKEADIDRLNEEGKLYLFQIYNKDFSEKSTGRENLHTMYLKNLFSE |
| | ENVREQVLKLNGEAEIFFRKSSVKKPIIHKKGTMLVNRTYMEEVNGNSV |
| | RRNIPEKEYQEIYNYKNHRLKGELSTEAKKYLEKAVCHETKKDIVKDY |
| | RYSVDKFFIHLPITINYRASGKETLNSVAQRYIAHQNDMHVIGIDRGERN |
| | LIYVSVINMQGEIKEQKSFNIINEFNYKEKLKEREQSRGAARRNWKEIG |
| | QIKDLKEGYLSGVIHEIAKMMIKYHAIIAMEDLNYGFKRGRFKVERQV |
| | YQKFENMLIQKLNYLVFKDRPADEDGGVLRGYQLAYIPDSVKKMGRQ |
| | CGMIFYVPAAFTSKIDPTTGFVDIFKHKVYTTEQAKREFILSFDEICYDVE |
| | RQLFRFTFDYANFVTQNVTLARNNWTIYTNGTRAQKEFGNGRMRDKE |
| | DYNPKDKMVELLESEGIEFKSGKNLLPALKKVSNAKVFEELQKIVRFTV |
| | QLRNSKSEENDVDYDHVISPVLNEEGNFFDSSKYKNKEEKKESLLPVDA |
| | DANGAYCIALKGLYIMQAIQKNWSEEKALSPDVLRLNNNDWFDYIQNK |
| | RYR* |
| SEQ ID | MEEKKMSKIEKFIGKYKISKTLRFRAVPVGKTQDNIEKKGILEKDKKRSE |
| NO: 214 | DYEKVKAYLDSLHRDFIENTLKKVKLNELNEYACLFFSGTKDDGDKKK |
| | MEKLEEKMRKTISNEFCNDEMYKKIFSEKILSENNEEDVSDIVSSYKGFF |
| | TSLNGYVNNRKNLYVSDAKPTSIAYRCINENLPKFLRNVECYKKVVQVI |
| | PKEQIEYMSNNLNLSPYRIEDCFNIDFFEFCLSQGGIDLYNTFIGGYSKKD |
| | GTKVQGINEIVNLYNQKNKKDKEKYKLPQFTPLFKQILSDRDTKSFSIEK |
| | LENIYEVVELVKKSYSDEMFDDIETVFSNLNYYDASGIYVKNGPAITHIS |
| | MNLTKDWATIRNNWNYEYDEKHSTKKNKNIEKYEDTRNTMYKKIDSF |
| | TLEYISRLVGKDIDELVKYFENEVANFVMDIKKTYSKLTPLFDRCQKENF |
| | DISEDEVNDIKGYLDNVKLLESFMKSFTINGKENNIDYVFYGKFTDDYD |
| | KLHEFDHIYNKVRNYITTSRKPYKLDKYKLYFDNPQLLGGWDINKEKD |
| | YRTVMLTKDGKYYFAIIDKGEHPFDNIPKDYFDNNGYYKKIIYRQIPNA |
| | AKYLSSKQIVPQNPPEEVKRILDKKKADSKSLTEEEKNIFIDYIKSDFLKN |
| | YKLLFDKNNNPYFNFAFRESSTYESLNEFFEDVERQAYSVRYENLPADYI |
| | DNLVNEGKIYLFEIYSKDFSEYSKGTNNLHTMYFKALFDNDNLKNTVF |
| | KLSGNAELFIRPASIKKDELVIHPKNQLLQNKNPLNPKKQSIFDYDLVKD |
| | KRFFENQYMLHISIEINKNERDAKKIKNINEMVRKELKDSDDNYIIGIDR |
| | GERNLLYVCVINSAGKIVEQMSLNEIINEYNGIKHTVDYQGLLDKCEKE |
| | RNAQRQSWKSIENIKELKDGYISQVVHKLCQLVEKYDAIIAMENLNGGF |
| | KRGRTKFEKQVYQKFENKLINKMEYMADKKRKTTENGGILRGYQLTN |
| | GCINNSYQNGFIFYVPAWLTSKIDPTTGFVDLLKPKYTNVEEAHLWINKF |
| | NSITYDKKLDMFAFNINYSQFPRADIDYRKIWTFYTNGYRIETFRNSEKN |
| | NEFDWKEVHLTSVIKKLLEEYQINYISGKNIIDDLIQIKDKPFWNSFIKYI |
| | RLTLQMRNSITGRTDVDYIISPVINNEGTFYDSRKDLDEITLPQDADANG |
| | AYNIARKALWIIEKLKESPDEELNKVKLAITQREWLEYAQINI* |
| SEQ ID | MEKIKKPSNRNSIPSIIISDYDANKIKEIKVKYLKLARLDKITIQDMEIVD |
| NO: 215 | NIVEFKKILLNGVEHTIIDNQKIEFDNYEITGCIKPSNKRRDGRISQAKYV |
| | VTITDKYLRENEKEKRFKSTERELPNNTLLSRYKQISGFDTLTSKDIYKIK |
| | RYIDFKNEMLFYFQFIEEFFNPLLPKGKNFYDLNIEQNKDKVAKFIVYRL |
| | NDDFKNKSLNSYITDTCMIINDFKKIQKILSDFRHALAHFDFDFIQKFFD |
| | DQLDKNKFDINTISLIETLLDQKEEKNYQEKNNYIDDNDILTIFDEKGSK |
| | FSKLHNFYTKISQKKPAFNKLINSFLSQDGVPNEEFKSYLVTKKLDFFEDI |
| | HSNKEYKKIYIQHKNLVIKKQKEESQEKPDGQKLKNYNDELQKLKDEM |
| | NTITKQNSLNRLEVKLRLAFGFIANEYNYNFKNFNDEFTNDVKNEQKIK |
| | AFKNSSNEKLKEYFESTFIEKRFFHFSVNFFNKKTKKEETKQKNIFNSIEN |
| | ETLEELVKESPLLQIITLLYLFIPRELQGEFVGFILKIYHHTKNITSDTKEDE |
| | ISIEDAQNSFSLKFKILAKNLRGLQLFHYSLSHNTLYNNKQCFFYEKGNR |
| | WQSVYKSFQISHNQDEFDIHLVIPVIKYYINLNKLMGDFEIYALLKYADK |
| | NSITVKLSDITSRDDLKYNGHYNFATLLFKTFGIDTNYKQNKVSIQNIKK |
| | TRNNLAHQNIENMLKAFENSEIFAQREEIVNYLQTEHRMQEVLHYNPIN |
| | DFTMKTVQYLKSLSVHSQKEGKIADIHKKESLVPNDYYLIYKLKAIELL |
| | KQKVIEVIGESEDEKKIKNAIAKEEQIKKGNN |
| SEQ ID | MEKSLNDFIGLYSVSKTLRFELKPVSETLENIKKFHFLEEDKKKANDYK |
| NO: 216 | DVKKIIDNYHKYFIDDVLKNASFNWKKLEEAIREYNKNKSDDSALVAE |
| | QKKLGDAILKLFTSDKRYKALTAATPKELFESILPDWFGEQCNQDLNKA |
| | ALKTFQKFTSYFTGFQENRKNVYSAEAIPTAVPYRIVNDNFPKFLQNVLI |
| | FKTIQEKCPQIIDEVEKELSSYLGKEKLAGIFTLESFNKYLGQGGKENQR |
| | GIDFYNQIIGGVVEKEGGINLRGVNQFLNLYWQQHPDFTKEDRRIKMVP |
| | LYKQILSDRSSLSFKIESIENDEELKNALLECADKLELKNDEKKSIFEEVC |
| | DLFSSVKNLDLSGIYINRKDINSVSRILTGDWSWLQSRMNVYAEEKFTT |
| | KAEKARWQKSLDDEGENKSKGFYSLTDLNEVLEYSSENVAETDIRITDY |
| | FEHRCRYYVDKETEMFVQGSELVALSLQEMCDDILKKRKAMNTVLENL |
| | SSENKLREKTDDVAVIKEYLDAVQELLHRIKPLKVNGVGDSTFYSVYDSI |
| | YSALSEVISVYNKTRNYITKKAASPEKYKLNFDNPTLADGWDLNKEQA |
| | NTSVILRKDGMFYLGIMNPKNKPKFAEKYDCGNESCYEKMIYKQFDAT |
| | KQIPKCSTQKKEVQKYFLSGATEPYILNDKKSFKSELIITKDIWFMNNHV |
| | WDGEKFVPKRDNETRPKKFQIGYFKQTGDFDGYKNALSNWISFCKNFL |
| | QSYLSATVYDYNFKNSEEYEGLDEFYNYLNATCYKLNFINIPETEINKM |
| | VSEGKLYLFQIYNKDFASGSTGMPNMHTLYWKNLFSDENLKNVCLKLN |
| | GEAELFYRPAGIKEPVIHKEGSYLVNRTTEDGESIPEKIYFEIYKNANGKL |
| | EKLSDEAQNYISNHEVVIKKAGHEIIKDRHYTEPKFLFHVPLTINFKASG |
| | NSYSINENVRKFLKNNPDVNIIGLDRGERHLIYLSLINQKGEIIKQFTFNE |
| | VERNKNGRTIKVNYHEKLDQREKERDAARKSWQAIGKIAELKEGYLSA |
| | VIHQLTKLMVEYNAVVVMEDLNFGFKRGRFHVEKQVYQKFEHILIDKS |
| | NYLVFKDRGLNEPGGVLNGYQIAGQFESFQKLGKQSGMLFYVPAGYTS |
| | KIDPKTGFVSMMNFKDLTNVHKKRDFFSKFDNIHYDEANGSFVFTFDY |
| | KKFDGKAKEEMKLTKWSVYSRDKRIVYFAKTKSYEDVLPTEKLQKIFE |
| | SNGIDYKSGNNIQDSVMAIGADLKEGAKPSKEISDFWDGLLSNFKLILQ |
| | MRNSNARTGEDYIISPVMADDGTFFDSREEFKKGEDAKLPLDADANGA |
| | YHIALKGLSLINKINLSKDEELKKFDMKISNADWFKFAQEKNYAK* |
| SEQ ID | MENYGGFTGLYPLQKTLKFELRPQGRTMEHLVSSNFFEEDRDRAEKYKI |
| NO: 217 | VKKVIDNYHKDFINECLSKRSFDWTPLMKTSEKYYASKEKNGKKKQDL |
| | DQKIIPTIENLSEKDRKELELEQKRMRKEIVSVFKEDKRFKYLFSEKLFSI |
| | LLKDEDYSKEKLTEKEILALKSFNKFSGYFIGLHKNRANFYSEGDESTAI |
| | AYRIVNENFPKFLSNLKKYREVCEKYPEIIQDAEQSLAGLNIKMDDIFPM |
| | ENFNKVMTQDGIDLYNLAIGGKAQALGEKQKGLNEFLNEVNQSYKKG |
| | NDRIRMTPLFKQILSERTSYSYILDAFDDNSQLITSINGFFTEVEKDKEGN |
| | TFDRAVGLIASYMKYDLSRVYIRKADLNKVSMEIFGSWERLGGLLRIFK |
| | SELYGDVNAEKTSKKVDKWLNSGEFSLSDVINAIAGSKSAETFDEYILK |
| | MRVARGEIDNALEKIKCINGNFSEDENSKMIIKAILDSVQRLFHLFSSFQV |
| | RADFSQDGDFYAEYNEIYEKLFAIVPLYNRVRNYLTKNNLSMKKIKLNF |
| | KNPALANGWDLNKEYDNTAVIFLREGKYYLGIMNPSKKKNIKFEEGSG |
| | TGPFYKKMAYKLLPDPNKMLPKVFFAKKNINYYNPSDEIVKGYKAGKY |
| | KKGENFDIDFCHKLIDFFKESIQKNEDWRAFNYLFSATESYKDISDFYSE |
| | VEDQGYRMYFLNVPVANIDEYVEKGDLFLFQIYNKDFASGAKGNKDM |
| | HTIYWNAAFSDENLRNVVVKLNGEAELFYRDKSIIEPICHKKGEMLVNR |
| | TCFDKTPVPDKIHKELFDYHNGRAKTLSIEAKGYLDRVGVFQASYEIIK |
| | DRRYSENKMYFHVPLKLNFKADGKKNLNKMVIEKFLSDKDVHIIGIDR |
| | GERNLLYYSVIDRRGNIIDQDSLNIIDGFDYQKKLGQREIERREARQSWN |
| | SIGKIKDLKEGYLSKAVHKVSKMVLEYNAIVVLEDLNFGFKRGRFKVE |
| | KQVYQKFEKMLIDKLNYLVFKEVLDSRDAGGVLNAYQLTTQLESFNKL |
| | GKQSGILFYVPAAYTSKIDPTTGFVSLFNTSRIESDSEKKDFLSGFDSIVYS |
| | AKDGGIFAFKFDYRNRNFQREKTDHKNIWTVYTNGDRIKYKGRMKGY |
| | EITSPTKRIKDVLSSSGIRYDDGQELRDSIIQSGNKVLINEVYNSFIDTLQ |
| | MRNSDGEQDYIISPVKNRNGEFFRTDPDRRELPVDADANGAYHIALRGE |
| | LLMQKIAEDFDPKSDKFTMPKMEHKDWFEFMQTRGD* |
| SEQ ID | MEVQKTVMKTLSLRILRPLYSQEIEKEIKEEKERRKQAGGTGELDGGFY |
| NO: 218 | KKLEKKHSEMFSFDRLNLLLNQLQREIAKVYNHAISELYIATIAQGNKSN |
| | KHYISSIVYNRAYGYFYNAYIALGICSKVEANFRSNELLTQQSALPTAKS |
| | DNFPIVLHKQKGAEGEDGGFRISTEGSDLIFEIPIPFYEYNGENRKEPYK |
| | WVKKGGQKPVLKLILSTFRRQRNKGWAKDEGTDAEIRKVTEGKYQVS |
| | QIEINRGKKLGEHQKWFANFSIEQPIYERKPNRSIVGGLDVGIRSPLVCAI |
| | NNSFSRYSVDSNDVFKFSKQVFAFRRRLLSKNSLKRKGHGAAHKLEPIT |
| | EMTEKNDKFRKKIIERWAKEVTNFFVKNQVGIVQIEDLSTMKDREDHFF |
| | NQYLRGFWPYYQMQTLIENKLKEYGIEVKRVQAKYTSQLCSNPNCRY |
| | WNNYFNFEYRKVNKFPKFKCEKCNLEISADYNAARNLSTPDIEKFVAK |
| | ATKGINLPEK* |
| SEQ ID | MIIHNCYIGGSFMKKIDSFTNCYSLSKTLRFKLIPIGATQSNFDLNKMLDE |
| NO: 219 | DKKRAENYSKAKSIIDKYHRFFIDKVLSSVTENKAFDSFLEDVRAYAELY |
| | YRSNKDDSDKASMKTLESKMRKFIALALQSDEGFKDLFGQNLIKKTLP |
| | EFLESDTDKEIIAEFDGFSTYFTGFFNNRKNMYSADDQPTAISYRCINDN |
| | LPKFLDNVRTFKNSDVASILNDNLKILNEDFDGIYGTSAEDVFNVDYFPF |
| | VLSQKGIEAYNSILGGYTNSDGSKIKGLNEYINLYNQKNENIHRIPKMKQ |
| | LFKQILSERESVSFIPEKFDSDDDVLSSINDYYLERDGGKVLSIEKTVEKI |
| | EKLFSAVTDYSTDGIFVKNAAELTAVCSGAFGYWGTVQNAWNNEYDAL |
| | NGYKETEKYIDKRKKAYKSIESFSLADIQKYADVSESSETNAEVTEWLR |
| | NEIKEKCNLAVQGYESSKDLISKPYTESKKLFNNDNAVELIKNALDSVK |
| | ELENVLRLLLGTGKEESKDENFYGEFLPCYERICEVDSLYDKVRNYMTQ |
| | KLYKTDKIKLNFQNPQFLGGWDRNKEADYSAVLLRRNSLYYIAIMPSGY |
| | KRVFEKIPAPKADETVYEKVIYKLLPGPNKMLPKVFFSKKGIETFNPPKE |
| | ILEKYELGTHKTGDGFNLDDCHALIDYFKSALDVHSDWSNFGFRFSDTS |
| | TYKNIADFYNEVKNQGYKITFCDVPQSYINELVDEGKLYLFQLYNKDFS |
| | EHSKGTPNLHTLYFKMLFDERNLENVVFKLNGEAEMFYREASISKDDM |
| | IVHPKNQPIKNKNEQNSRKQSTFEYDIVKDRRYTVDQFMLHIPITLNFTA |
| | NGGTNINNEVRKALKDCDKNYVIGIDRGERNLLYICVVDSEGRIIEQYSL |
| | NEIINEYNGNTYSTDYHALLDKKEKERLESRKAWKTVENIKELKEGYIS |
| | QVVHKICELVEKYDAVIVMEDLNLGFKQGRSGKFEKSVYQKFEKMLID |
| | KLNYFADKKKSPEEIGSVLNAYQLTNAFESFEKMGKQNGFIFYVPAYLTS |
| | KIDPTTGFADLLHPSSKQSKESMRDFVGRFDSITFNKTENYFEFELDYNK |
| | FPRCNTDYRKKWTVCTYGSRIKTFRNPEKNSEWDNKTVELTPAFMALF |
| | EKYSIDVNGDIKAQIMSVDKKDFFVELIGLLRLTLQMRNSETGKVDRDY |
| | LISPVKNSEGVFYNSDDYKGIENASLPKDADANGAYNIARKGLWIIEQIK |
| | ACENDAELNKIRLAISNAEWLEYAQKK* |
| SEQ ID | MKDYIRKTLSLRILRPYYGEEIEKEIAAAKKKSQAEGGDGALDNKFWD |
| NO: 220 | RLKAEHPEIISSREFYDLLDAIQRETTLYYNRAISKLYHSLIVEREQVSTA |
| | KALSAGPYHEFREKFNAYISLGLREKIQSNFRRKELARYQVALPTAKSDT |
| | FPIPIYKGFDKNGKGGFKVREIENGDFVIDLPLMAYHRVGGKAGREYIEL |
| | DRPPAVLNVPVILSTSRRRANKTWFRDEGTDAEIRRVMAGEYKVSWVEI |
| | LQRKRFGKPYGGWYVNFTIKYQPRDYGLDPKVKGGIDIGLSSPLVCAVT |
| | NSLARLTIRDNDLVAFNRKAMARRRTLLRQNRYKRSGHGSANKLKPIEA |
| | LTEKNELYRKAIMRRWAREAADFFRQHRAATVNMEDLTGIKDREDYFS |
| | QMLRCYWNYSQLQTMLENKLKEYGIAVKYIEPKDTSKTCHSCGHVNE |
| | YFDFNYRSAHKFPMFKCEKCGVECGADYNAARNIAQA |
| SEQ ID | MKEQFINRYPLSKTLRFSLIPVGETENNFNKNLLLKKDKQRAENYEKVK |
| NO: 221 | CYIDRFHKEYIESVLSKARIEKVNEYANLYWKSNKDDSDIKAMESLEND |
| | MRKQISKQLTSTEIYKKRLFGKELICEDLPSFLTDKDERETVECFRSFTTY |
| | FKGFNTNRENMYSSDGKSTAIAYRCINDNLPRFLDNVKSFQKVFDNLSD |
| | ETITKLNTDLYNIFGRNIEDIFSVDYFEFVLTQSGIEIYNSMIGGYTCSDKT |
| | KIQGLNECINLYNQQVAKNEKSKKLPLMKPLYKQILSEKDSVSFIPEKFN |
| | SDNEVLHAIDDYYTGHIGDFDLLTELLQSLNTYNANGIFVKSGVAITDIS |
| | NGAFNSWNVLRSAWNEKYEALHPVTSKTKIDKYIEKQDKIYKAIKSFSL |
| | FELQSLGNENGNEITDWYISSINESNSKIKEAYLQAQKLLNSDYEKSYNK |
| | RLYKNEKATELVKNLLDAIKEFQKLIKPLNGTGKEENKDELFYGKFTSY |
| | YDSIADIDRLYDKVRNYITQKPYSKDKIKLNFDNPQLLGGWDKNKESD |
| | YRTVLLHKDGLYYLAVMDKSHSKAFVDAPEITSDDKDYYEKMEYKLLP |
| | GPNKMLPKVFFASKNIDTFQPSDRILDIRKRESFKKGATFNKAECHEFID |
| | YFKDSIKKHDDWSQFGFKFSPTESYNDISEFYREISDQGYSVRFNKISKN |
| | YIDGLVNNGYIYLFQIYNKDFSKYSKGTPNLHTLYFKMLFDERNLSNVV |
| | YKLNGEAEMFYREASIGDKEKITHYANQPIKNKNPDNEKKESVFEYDIV |
| | KDKRFTKRQFSLHLPITINFKAHGQEFLNYDVRKAVKYKDDNYVIGIDR |
| | GERNLIYISVINSNGEIVEQMSLNEIISDNGHKVDYQKLLDTKEKERDKA |
| | RKNWTSVENIKELKEGYISQVVHKICELVIKYDAVIAMEDLNFGFKRGR |
| | FPVEKQVYQKFENMLISKLNLLIDKKAEPTEDGGLLRAYQLTNKFDGVN |
| | KAKQNGIIFYVPAWDTSKIDPATGFVNLLKPKCNTSVPEAKKLFETIDDI |
| | KYNANTDMFEFYIDYSKFPRCNSDFKKSWTVCTNSSRILTFRNKEKNNK |
| | WDNKQIVLTDEFKSLFNEFGIDYKGNLKDSILSISNADFYRRLIKLLSLTL |
| | QMRNSITGSTLPEDDYLISPVANKSGEFYDSRNYKGTNAALPCDADANG |
| | AYNIARKALWAINVLKDTPDDMLNKAKLSITNAEWLEYTQK* |
| SEQ ID | MKEQFVNQYPISKTLRFSLIPIGKTEENFNKNLLLKEDEKKAEEYQKVK |
| NO: 222 | GYIDRYHKFFIETALCNINFEGFEEYSLLYYKCSKDDNDLKTMEDIEIKL |
| | RKQISKTMTSHKLYKDLFGENMIKTILPNFLDSDEEKNSLEMFRGFYTY |
| | FSGFNTNRKNMYTEEAKSTSIAYRCINDNLPKFLDNSKSFEKIKCALNKE |
| | ELKAKNEEFYEIFQIYATDIFNIDFFNFVLTQPGIDKYNGIIGGYTCSDGTK |
| | VQGLNEIINLYNQQIAKDDKSKRLPLLKMLYKQILSDRETVSFIPEKFSSD |
| | NEVLESINNYFSKNVSNAIKSLKELFQGFEAYNMNGIFISSGVAITDLSNA |
| | VFGDWNAISTAWEKAYFETNPPKKNKSQEKYEEELKANYKKIKSFSLDE |
| | IQRLGSIAKSPDSIGSVAEYYKITVTEKIDNITELYDGSKELLNCNYSESY |
| | DKKLIKNDTVIEKVKTLLDAVKSLEKLIKPLVGTGKEDKDELFYGTFLPL |
| | YTSLSAVDRLYDKVRNYATQKPYSKDKIKLNFNCSSFLSGWATDYSSNG |
| | GLIFEKDGLYYLGIVNKKFTTEEIDYLQQNADENPAQRIVYDFQKPDNK |
| | NTPRLFIRSKGTNYSPSVKEYNLPVEEIVELYDKRYFTTEYRNKNPELYK |
| | ASLVKLIDYFKLGFTRHESYRHYDFKWKKSEEYNDISEFYKDVEISCYS |
| | LKQEKINYNTLLNFVAENRIYLFQIYNKDFSKYSKGTPNLHTRYFKALFD |
| | ENNLSDVVFKLNGGSEMFFRKASIKDNEKVVHPANQPIDNKNPDNSKK |
| | QSTFDYELIKDKRFTKHQFSIHIPITMNFKARGRDFINNDIRKAIKSEYKP |
| | YVIGIDRGERNLIYISVINNNGEIVEQMSLNDIISDNGYKVDYQRLLDRK |
| | EKERDNARKSWGTIENIKELKEGYISQVIHKICELVIKYDAVIAMEDLNF |
| | GFKRGRFNVEKQVYQKFENMLISKLNYLCDKKSEANSEGGLLKAYQLT |
| | NKFDGVNKGKQNGIIFYVPAWLTSKIDPVTGFVDLLHPKYISVEETHSLF |
| | EKLDDIRYNFEKDMFEFDIDYSKLPKCNADFKQKWTVCTNADRIMTFR |
| | NSEKNNEWDNKRILLSDEFKRLFEEFGIDYCHNLKNKILSISNKDFCYRF |
| | IKLFALTMQMRNSITGSTNPEDDYLISPVRDENGVFYDSRNFIGSKAGLPI |
| | DADANGAYNIARKGLWAINAIKSTADDMLDKVDLSISNAKWLEYVQK* |
| SEQ ID | MKITKIDGILHKKYIKEGKLVKSTSEENKTDERLSELLTIRLDTYIKNPDN |
| NO: 223 | ASEEENRIRRETLKEFFSNKVLYLKDSILYLKDRREKNQLQNKNYSEEDI |
| | SEYDLKNKNSFLVLKKILLNEDINSEELEIFRNDFEKKLDKINSLKYSLEE |
| | NKANYQKINENNIKKVEGKSKRNIFYNYYKDSAKRNDYINNIQEAFDK |
| | LYKKEDIENLFFLIENSKKHEKYKIRECYHKIIGRKNDKENFATIIYEEIQN |
| | VNNMKELIEKVPNVSELKKSQVFYKYYLNKEKLNDENIKYVFCHFVEI |
| | EMSKLLKNYVYKKPSNISNDKVKRIFEYQSLKKLIENKLLNKLDTYVRN |
| | CGKYSFYLQDGEIATSDFIVGNRQNEAFLRNIIGVSSTAYFSLRNILETEN |
| | ENDITGRIKGKTVKNKKGEEKYISGEIDKLYDNNKQNEVKKNLKMFYS |
| | YDFNMNRKKEIEDFFSNIDEAISSIRHGIVHFNLELEGKDIFTFKNIVPSQI |
| | SKKMFQNEINEKKLKLKIFRQLNSANVFRYLEKYKILNYLNRTRFEFVN |
| | KNIPFVPSFTKLYSRIDDLKNSLCIYWKIPKANDNNKTKEITDAQIYLLK |
| | NIYYGEFLNYFMSNNGNFFEIIKEIIELNKNDKRNLKTGFYKLQKFENLQ |
| | EKTPKEYLANIQSFYMIDAGNKDEEEKDAYIDFIQKIFLKGFMTYLANN |
| | GRLSLMYIGNDEQINTSLAGKKQEFDKFLKKYEQNNNIEIPHEINEFVRE |
| | IKLGKILKYTESLNMFYLILKLLNHKELTNLKGSLEKYQSANKEEAFSD |
| | QLELINLLNLDNNRVTEDFELEADEIGKFLDFNGNKVKDNKELKKFDTN |
| | KIYFDGENIIKHRAFYNIKKYGILNLLEKISDEAKYKISIEELKNYSNKKIE |
| | IEKNHTTQENLHRKYARPRKDEKFNDEDYKKYEKTIRNIQQYTHLKNK |
| | VEFNELNLLQSLLLRILHRLVGYTSIWERDLRFRLKGEFPENQYIEEIFNF |
| | DNSKNVKYKNGQIVEKYISFYKELYKDDMEKISIYSDKKVKELKKEKK |
| | DLYIRNYIAHFNYIPNAEVSLLEVLENLRKLLSYDRKLKNAIMKSIVDIL |
| | KEYGFVVTFKIEKDKKIRIESLKSEEVVHLKKLKLKDNDKKKEPIKTYR |
| | NSKELCKLVKVMFEYKMKEKKSEN* |
| SEQ ID | MKITKIDGISHKKYIKEGKLVKSTSEENKTDERLSELLTIRLDTYIKNPDN |
| NO: 224 | ASEEENRIRRENLKEFFSNKVLYLKDGILYLKDRREKNQLQNKNYSEEDI |
| | SEYDLKNKNSFLVLKKILLNEDINSEELEIFRKDVEAKLNKINSLKYSFEE |
| | NKANYQKINENNVEKVGGKSKRNIIYDYYRESAKRNDYINNVQEAFDK |
| | LYKKEDIEKLFFLIENSKKHEKYKIRECYHKIIGRKNDKENFAKIIYEEIQ |
| | NVNNIKELIEKVPDMSELKKSQVFYKYYLDKEELNDKNIKYAFCHFVEI |
| | EMSQLLKNYVYKRLSNISNDKIKRIFEYQNLKKLIENKLLNKLDTYVRN |
| | CGKYNYYLQDGEIATSDFIAGNRQNEAFLRNIIGVSSVAYFSLRNILETEN |
| | KDDITGKMRGKTRIDSKTGEEKYIPGEVDQIYYENKQNEVKNKLKMFY |
| | GYDFDMDNKKEIEDFFANIDEAISSIRHGIVHFNLDLDGKDIFAFKNIVPS |
| | EISKKMFQNEINEKKLKLKIFRQLNSANVFRYLEKYKILNYLKRTRFEFV |
| | NKNIPFVPSFTKLYSRIDDLKNSLGIYWKTPKTNDDNKTKEIIDAQIYLL |
| | KNIYYGEFLNYFMSNNGNFFEISREIIELNKNDKRNLKTGFYKLQKFEDI |
| | QEKTPKKYLANIQSLYMINAGNQDEEEKDTYIDFIQKIFLKGFMTYLAN |
| | NGRLSLMYIGNDEQINTSLAGKKQEFDKFLKKYEQNNNIEIPHEINEFLR |
| | EIKLGKILKYTESLNMFYLILKLLNHKELTNLKGSLEKYQSANKEETFSD |
| | ELELINLLNLDNNRVTEDFELEANEIGKFLDFNGNKIKDRKELKKFDTK |
| | KIYFDGENIIKHRAFYNIKKYGMLNLLEKIADKAKYKISLKELKEYSNK |
| | KNEIEKNYTMQQNLHRKYARPKKDEKFNDEDYKEYEKAIGNIQKYTHL |
| | KNKVEFNELNLLQGLLLKILHRLVGYTSIWERDLRFRLKGEFPENQYIEE |
| | IFNFDNSKNVKYKSGQIVEKYINFYKELYKDNVEKRSIYSDKKVKKLKQ |
| | EKKDLYIRNYIAHFNYIPHAEISLLEVLENLRKLLSYDRKLKNAIMKSVV |
| | DILKEYGFVATFKIGADKKIGIQTLESEKIVHLKNLKKKKLMTDRNSEEL |
| | CKLVKVMFEYKMEEKNLKTKKCKVI* |
| SEQ ID | MKKIDNFVGCYPVSKTLRFKAIPIGKTQENIEKKRLVEEDEVRAKDYKA |
| NO: 225 | VKKLIDRYHREFIEGVLDNVKLDGLEEYYMLFNKSDREESDNKKIEIME |
| | ERFRRVISKSFKNNEEYKKIFSKKIIEEILPNYIKDEEEKELVKGFKGFYT |
| | AFVGYAQNRENMYSDEKKSTAISYRIVNENMPRFITNIKVFEKAKSILD |
| | VDKINEINEYILNNDYYVDDFFNIDFFNYVLNQKGIDIYNAIIGGIVTGD |
| | GRKIQGLNECINLYNQENKKIRLPQFKPLYKQILSESESMSFYIDEIESDD |
| | MLIDMLKESLQIDSTINNAIDDLKVLFNNIFDYDLSGIFINNGLPITTISND |
| | VYGQWSTISDGWNERYDVLSNAKDKESEKYFEKRRKEYKKVKSFSISD |
| | LQELGGKDLSICKKINEIISEMIDDYKSKIEEIQYLFDIKELEKPLVTDLNK |
| | IELIKNSLDGLKRIERYVIPFLGTGKEQNRDEVFYGYFIKCIDAIKEIDGV |
| | YNKTRNYLTKKPYSKDKFKLYFENPQLMGGWDRNKESDYRSTLLRKN |
| | GKYYVAIIDKSSSNCMMNIEEDENDNYEKINYKLLPGPNKMLPKVFFSK |
| | KNREYFAPSKEIERIYSTGTFKKDTNFVKKDCENLITFYKDSLDRHEDW |
| | SKSFDFSFKESSAYRDISEFYRDVEKQGYRVSFDLLSSNAVNTLVEEGK |
| | LYLFQLYNKDFSEKSHGIPNLHTMYFRSLFDDNNKGNIRLNGGAEMFM |
| | RRASLNKQDVTVHKANQPIKNKNLLNPKKTTTLPYDVYKDKRFTEDQ |
| | YEVHIPITMNKVPNNPYKINHMVREQLVKDDNPYVIGIDRGERNLIYVV |
| | VVDGQGHIVEQLSLNEIINENNGISIRTDYHTLLDAKERERDESRKQWK |
| | QIENIKELKEGYISQVVHKICELVEKYDAVIALEDLNSGFKNSRVKVEK |
| | QVYQKFEKMLITKLNYMVDKKKDYNKPGGVLNGYQLTTQFESFSKMG |
| | TQNGIMFYIPAWLTSKMDPTTGFVDLLKPKYKNKADAQKFFSQFDSIR |
| | YDNQEDAFVFKVNYTKFPRTDADYNKEWEIYTNGERIRVFRNPKKNNE |
| | YDYETVNVSERMKELFDSYDLLYDKGELKETICEMEESKFFEELIKLFR |
| | LTLQMRNSISGRTDVDYLISPVKNSNGYFYNSNDYKKEGAKYPKDADA |
| | NGAYNIARKVLWAIEQFKMADEDKLDKTKISIKNQEWLEYAQTHCE |
| SEQ ID | MKKIDSFVNYYPLSKTLRFSLIPVGKTEDNFNAKLLLEEDEKRAIEYEK |
| NO: 226 | VKRYIDRYHKHFIETVLANFHLDDVNEYAELYYKAGKDDKDLKYMEK |
| | LEGKMRKSISAAFTKDKKYKEIFGQEIIKNILPEFLENEDEKESVKMFQG |
| | FFTYFTGFNDNRKNMYTHEAQTTAISYRCINENLPKFLDNVQSFAKIKES |
| | ISSDIMNKLDEVCMDLYGVYAQDMFCTDYFSFVLSQSGIDRYNNIIGGY |
| | VDDKGVKIQGINEYINLYNQQVDEKNKRLPLMKKLYKQILIEKESISFIPE |
| | KFESDNIVINAISDYYHNNVENLFDDFNKLFNEFSEYDDNGIFVTSGLAV |
| | TDISNAVFGSWNIISDSWNEEYKDSHPMKKTTNAEKYYEDMKKEYKKN |
| | LSFTIAELQRLGEAGCNDECKGDIKEYYKTTVAEKIENIKNAYEISKDLL |
| | ASDYEKSNDKKLCKNDSAISLLKNLLDSIKDLEKTIKPLLGTGKEENKD |
| | DVFYGKFTNLYEMISEIDRLYDKVRNYVTQKPYSKDKIKLNFENPQHLG |
| | GWDKNKERDYRSVLLKKEDKYYLAIMDKSNNKAFIDFPDDGECYEKIE |
| | YKLLPGPNKMLPKVFFASSNIEYFAPSKKILEIRSRESFKKGDMFNLKDC |
| | HEFIDFFKESIKKHEDWSQFGFEFSPTEKYNDISEFYNEVKIQGYSLKYK |
| | NVSKKYIDELIECGQLYLFQIYNKDFSVYAKGNPNLHTMYFKMLFDERN |
| | LANVVYQLNGGAEMFYRKASIKDSEKIVHHANQPIKNKNADNVKKES |
| | VFEYDIIKDKRFTKRQFSIHIPITLNFKAKGQNFINNDVRMALKKADENY |
| | VIGIDRGERNLLYICVINSKGEIVEQKSLNEIIGDNGYRVDYHKLLDKKE |
| | AERDEARKSWGTIENIKELKEGYLSQIVHEISKLVIKYDAVIAIEDLNSGF |
| | KKGRFKVEKQVYQKFENMLCTKLNYLVDKNADANECGGLLKAYQLT |
| | NKEDGANRGRQNGIIFSVPAWLTSKIDPVTGFADLLRPKYKSVSESVEFI |
| | SKIDNIRYNSKEDYFEFDIDYSKFPNSTASYKKKWTVCTYGERIINVRNK |
| | EKNNMWDNKTIVLTDEFKKLFADFGVDVSKNIKESVLAIDSKDFYYRFI |
| | NLLANTLQLRNSEVGNVDVDYLISPVKGVDGSFYDSRLVKEKTLPENA |
| | DANGAYNIARKALWAIDVLKQTKDEELKNANLSIKNAEWLEYVQK* |
| SEQ ID | MKNQNTLPSNPTDILKDKPFWAAFFNLARHNVYLTVNHINKLLDLEKL |
| NO: 227 | YNKDKHKEIFEHEDIFNISDDVMNDVNSNGKKRKLDIKKIWANLDTDLT |
| | RKYQLRELILKHFPFIQPAIIGAQTKERTTIDKDKRSTSTSNDSLKPTGEG |
| | DINDPLSLSNVKSIFFRLLQMLEQLRNYYSHVKHSKSATMPNFDEGLLK |
| | SMYNIFIDSVNKVKEDYSSNSVIDPNTSFSHLISKDEQGEIKPCRYSFTSK |
| | DGSINASGLLFFVSLFLEKQDSIWMQKKIPGFKKTSENYMKMTNEVFCR |
| | NHILLPKMRLETVYDKDWMLLDMLNEVVRCPLSLYKRLAPADQNKFK |
| | VPEKSSDNANRQEDDNPFSRILVRHQNRFPYFALRFFDLNEVFTTLRFQI |
| | NLGCYHFAICKKQIGDKKEVHHLTRTLYGFSRLQNFTQNTRPEEWNTLV |
| | KTTEPSSGNDGKTVQGVPLPYISYTIPHYQIENEKIGIKIFDGDTAVDTDI |
| | WPSVSTEKQLNKPDKYTLTPGFKADVFLSVHELLPMMFYYQLLLCEGM |
| | LKTDAGNAVEKVLIDTRNAIFNLYDAFVQEKINTITDLENYLQDKPILIG |
| | HLPKQMIDLLKGHQRDMLKAVEQKKAMLIKDTERRLERLNKQPEQKP |
| | NVAAKNTGTLLRNGQIADWLVKDMMRFQPVKRDKEGNPINCSKANST |
| | EYQMLQRAFAFYTTDSYRLPRYFEQLHLINCDNSHLFLSRFEYDKQPNLI |
| | AFYAAYLEAKLEFLNELQPQNWASDNYFLLLRAPKNDRQKLAEGWKN |
| | GFNLPRGLFTEKIKTWFNEHKTIVDISDCDIFKNRVGQVARLIPVFFDKK |
| | FKDHSQPFYTYNFNVGNVSKITEANYLSKEKRENLFKSYQNKFKNNIPA |
| | EKTKEYREYKNFSSWKKFERELRLIKNQDILTWLMCKNLFDEKIKPKKD |
| | ILEPRIAVSYIKLDSLQTNTSTAGSLNALAKVVPMTLAIHIDSPKPKGKAG |
| | NNEKENKEFTVYIKEEGTKLLKWGNFKTLLADRRIKGLFSYIEHDDINL |
| | EKYPLTKYQVDSELDLYQKYRIDIFKQTLDLEAQLLDKYSDLNTDNFNQ |
| | MLSGWSEKEGIPRNIKQDVAFLIGVRNGFSHNQYPDSKRIAFSRIKKFNP |
| | KTSSLQESEGLNIAKQMYEEAQQVVNKIKNIESFD* |
| SEQ ID | MKVTKIDGISHKKFEDEGKLVKFTGHFNIKNEMKERLEKLKELKLSNYI |
| NO: 228 | KNPENVKNKDKNKEKETKSRRENLKKYFSEIILRKKEEKYLLKKTRKF |
| | KNITEEINYDDIKKRENQQKIFDVLKELLEQRINENDKEEILNFDSVKLK |
| | EAFEEDFIKKELKIKAIEESLEKNRADYRKDYVELENEKYEDVKGQNKR |
| | SLVFEYYKNPENREKFKENIKYAFENLYTEENIKNLYSEIKEIFEKVHLKS |
| | KVRYFYQNEIIGESEFSEKDEEGISILYKQIINSVEKKEKFIEFLQKVKIKD |
| | LTRSQIFYKYFLENEELNDENIKYVFSYFVEIEVNKLLKENVYKTKKFNE |
| | GNKYRVKNIFNYDKLKNLVVYKLENKLNNYVRNCGKYNYHMENGDI |
| | ATSDINMKNRQTEAFLRSILGVSSFGYFSLRNILGVNDDDFYKIEKDERK |
| | NENFILKKAKEDFTSKNIFEKVVDKSFEKKGIYQIKENLKMFYGNSFDK |
| | VDKDELKKFFVNMLEAITSVRHRIVHYNINTNSENIFDFSNIEVSKLLKN |
| | IFEKEIDTRELKLKIFRQLNSAGVFDYWESWVIKKYLENVKFEFVNKNV |
| | PFVPSFKKLYDRIDNLKGWNALKLGNNINIPKRKEAKDSQIYLLKNIYY |
| | GEFVEKFVNDNKNFEKIVKEIIEINRGAGTNKKTGFYKLEKFETLKANTP |
| | TKYLEKLQSLHKISYDKEKIEEDKDVYVDFVQKIFLKGFVNYLKKLDSL |
| | KSLNLLNLRKDETITDKKSVHDEKLKLWENSGSNLSKMPEEIYEYVKKI |
| | KISNINYNDRMSIFYLLLKLIDYRELTNLRGNLEKYESMNKNKIYSEELTI |
| | INLVNLDNNKVRTNFSLEAEDIGKFLKSSITIKNIAQLNNFSKIFADGENV |
| | IKHRSFYNIKKYGILDLLEKIVAKADLKITKEEIKKYENLQNELKRNDFY |
| | KIQEQIHRNYNQKPFSIKKIENKKDFEKYKKVIEKIQDYTQLKNKIEFND |
| | LNLLQSLIFRILHRLAGYTSLWERDLQFKLKGEFPEDKYIDEIFNSDGNN |
| | NOKYKHGGIADKYANFLIEKKEEKSGEILNKKQRKKKIKEDLEIRNYIA |
| | HFNYLPNAEKSILEILEELRELLKHDRKLKNAVMKSIKDIFREYGFIVEFT |
| | ISHTKNGKKIKVCSVKSEKIKHLKNNELITTRNSEDLCELVKIMLEHKEL |
| | QK* |
| SEQ ID | MKVTKVGGISHKKYTSEGRLVKSESEENRTDERLSALLNMRLDMYIKN |
| NO: 229 | PSSTETKENQKRIGKLKKFFSNKMVYLKDNTLSLKNGKKENIDREYSET |
| | DILESDVRDKKNFAVLKKIYLNENVNSEELEVFRNDIKKKLNKINSLKYS |
| | FEKNKANYQKINENNIEKVEGKSKRNIIYDYYRESAKRDAYVSNVKEAF |
| | DKLYKEEDIAKLVLEIENLTKLEKYKIREFYHEIIGRKNDKENFAKIIYEEI |
| | QNVNNMKELIEKVPDMSELKKSQVFYKYYLDKEELNDKNIKYAFCHFV |
| | EIEMSQLLKNYVYKRLSNISNDKIKRIFEYQNLKKLIENKLLNKLDTYVR |
| | NCGKYNYYLQDGEIATSDFIARNRQNEAFLRNIIGVSSVAYFSLRNILETE |
| | NENDITGRMRGKTVKNNKGEEKYVSGEVDKIYNENKKNEVKENLKMF |
| | YSYDFNMDNKNEIEDFFANIDEAISSIRHGIVHFNLELEGKDIFAFKNIAP |
| | SEISKKMFQNEINEKKLKLKIFRQLNSANVFRYLEKYKILNYLKRTRFEF |
| | VNKNIPFVPSFTKLYSRIDDLKNSLGIYWKTPKTNDDNKTKEIIDAQIYL |
| | LKNIYYGEFLNYFMSNNGNFFEISKEIIELNKNDKRNLKTGFYKLQKFE |
| | DIQEKIPKEYLANIQSLYMINAGNQDEEEKDTYIDFIQKIFLKGFMTYLA |
| | NNGRLSLIYIGSDEETNTSLAEKKQEFDKFLKKYEQNNNIKIPYEINEFLR |
| | EIKLGNILKYTERLNMFYLILKLLNHKELTNLKGSLEKYQSANKEEAFS |
| | DQLELINLLNLDNNRVTEDFELEADEIGKFLDFNGNKVKDNKELKKFDT |
| | NKIYFDGENIIKHRAFYNIKKYGMLNLLEKIADKAGYKISIEELKKYSNK |
| | KNEIEKNHKMQENLHRKYARPRKDEKFTDEDYESYKQAIENIEEYTHL |
| | KNKVEFNELNLLQGLLLRILHRLVGYTSIWERDLRFRLKGEFPENQYIEE |
| | IFNFENKKNVKYKGGQIVEKYIKFYKELHQNDEVKINKYSSANIKVLKQ |
| | EKKDLYIRNYIAHFNYIPHAEISLLEVLENLRKLLSYDRKLKNAVMKSV |
| | VDILKEYGFVATFKIGADKKIGIQTLESEKIVHLKNLKKKKLMTDRNSEE |
| | LCKLVKIMFEYKMEEKKSEN |
| SEQ ID | MNELVKNRCKQTKTICQKLIPIGKTRETIEKYNLMEIDRKIAANKELMN |
| NO: 230 | KLFSLIAGKHINDTLSKCTDLDFEPLLTSLSSLNNAKENDRDNLREYYDS |
| | VFEEKKTLAEEISSRLTAVKFAGKDFFTKNIPDFLETYEGDDKNEMSELV |
| | SLVIENTVTAGYVKKLEKIDRSMEYRLVSGTVVKRVLTDNADIYEKNIE |
| | KAKDFDYGVLNIDEASQFTTLVAKDYANYLTADGIAIYNVGIGKINLAL |
| | NEYCQKNKEYSYNKLALLPLQKMLYGEKLSLFEKLEDFTSDEELINSYN |
| | KFAKTVNESGLAEIIKKAVPSYDEIVIKPNKISNYSNSITGHWSLVNRIMK |
| | DYLENNGIKNADKYMEKGLTLSEIGDALENKNIKHSDFISNLINDLGHT |
| | YTEIKENKESLKKDESVNALIIKKELDMLLSILQNLKVFDIDNEMFDTGF |
| | GIEVSKAIEILGYGVPLYNKIRNYITKKPDPKKKFMTKFGSATIGTGITTS |
| | VEGSKKATFLKDGDAVFLLLYNTAGCKANNVSVSNLADLINSSLEIENS |
| | GKCYQKMIYQTPGDIKKQIPRVFVYKSEDDDLIKDFKAGLHKTDLSFLN |
| | GRLIPYLKEAFATHETYKNYTFSYRNSYESYDEFCEHMSEQAYILEWKW |
| | IDKKLIDDLVEDGSLLMFRVWNRFMKKKEGKISKHAKIVNELFSDENAS |
| | NAAIKLLSVFDIFYRDKQIDNPIVHKAGTTLYNKRTKDGEVIVDYTTMV |
| | KNKEKRPNVYTTTKKYDIIKDRRYTEEQFEIHLHVNIGKEENKEKLETS |
| | KVINEKKNTLVVTRSNEHLLYVVIFDENDNILLKKSLNTVKGMNFKSKL |
| | EVVEIQKKENMQSWKTVGSNQALMEGYLSFAIKEIADLVKEYDAILVLE |
| | QNSVGKNILNERVYTRFKEMLITNLSLDVDYENKDFYSYTELGGKVAS |
| | WRDCVTNGICIQVPSAYKYKDPTTSFSTISMYAKTTAEKSKKLKQIKSFK |
| | YNRERGLFELVIAKGVGLENNIVCDSFGSRSIIENDISKEVSCTLKIEKYLI |
| | DAGIEYNDEKEVLKDLDTAAKTDAVHKAVTLLLKCFNESPDGRYYISPC |
| | GEHFTLCDAPEVLSAINYYIRSRYIREQIVEGVKKMEYKKTILLAK* |
| SEQ ID | MNGNRIIVYREFVGVTPVAKTLRNELRPIGHTQEHIIHNGLIQEDELRQE |
| NO: 231 | KSTELKNIMDDYYREYIDKSLSGVTDLDFTLLFELMNLVQSSPSKDNKK |
| | ALEKEQSKMREQICTHMQSDSNYKNIFNAKFLKEILPDFIKNYNQYDAK |
| | DKAGKLETLALFNGFSTYFTDFFEKRKNVFTKEAVSTSIAYRIVHENSLT |
| | FLANMTSYKKISEKALDEIEVIEKNNQDKMGDWELNQIFNPDFYNMVLI |
| | QSGIDFYNEICGVVNAHMNLYCQQTKNNYNLFKMRKLHKQILAYTSTS |
| | FEVPKMFEDDMSVYNAVNAFIDETEKGNIIGKLKDIVNKYDELDEKRIYI |
| | SKDFYETLSCFMSGNWNLITGCVENFYDENIHAKGKSKEEKVKKAVKE |
| | DKYKSINDVNDLVEKYIDEKERNEFKNSNAKQYIREISNIITDTETAHLE |
| | YDEHISLIESEEKADEMKKRLDMYMNMYHWAKAFIVDEVLDRDEMFY |
| | SDIDDIYNILENIVPLYNRVRNYVTQKPYNSKKIKLNFQSPTLANGWSQS |
| | KEFDNNAIILIRDNKYYLAIFNAKNKPDKKIIQGNSDKKNDNDYKKMV |
| | YNLLPGANKMLPKVFLSKKGIETFKPSDYIISGYNAHKHIKTSENFDISF |
| | CRDLIDYFKNSIEKHAEWRKYEFKFSATDSYNDISEFYREVEMQGYRID |
| | WTYISEADINKLDEEGKIYLFQIYNKDFAENSTGKENLHTMYFKNIFSEE |
| | NLKDIIIKLNGQAELFYRRASVKNPVKHKKDSVLVNKTYKNQLDNGDV |
| | VRIPIPDDIYNEIYKMYNGYIKENDLSEAAKEYLDKVEVRTAQKDIVKD |
| | YRYTVDKYFIHTPITINYKVTARNNVNDMAVKYIAQNDDIHVIGIDRGE |
| | RNLIYISVIDSHGNIVKQKSYNILNNYDYKKKLVEKEKTREYARKNWKS |
| | IGNIKELKEGYISGVVHEIAMLMVEYNAIIAMEDLNYGFKRGRFKVERQ |
| | VYQKFESMLINKLNYFASKGKSVDEPGGLLKGYQLTYVPDNIKNLGKQ |
| | CGVIFYVPAAFTSKIDPSTGFISAFNFKSISTNASRKQFFMQFDEIRYCAE |
| | KDMFSFGFDYNNFDTYNITMSKTQWTVYTNGERLQSEFNNARRTGKT |
| | KSINLTETIKLLLEDNEINYADGHDVRIDMEKMDEDKNSEFFAQLLSLYK |
| | LTVQMRNSYTEAEEQEKGISYDKIISPVINDEGEFFDSDNYKESDDKECK |
| | MPKDADANGAYCIALKGLYEVLKIKSEWTEDGFDRNCLKLPHAEWLDF |
| | IQNKRYE* |
| SEQ ID | MNKDIRKNFTDFVGISEIQKTLRFILIPIGKTAQNIDKYNMFEDDEIRHEY |
| NO: 232 | YPILKEACDDFYRNHIDQQFENLELDWSKLDEALASEDRDLINETRATY |
| | RQVLFNRLKNSVDIKGDSKKNKTLSLESSDKNLGKKKTKNTFQYNFND |
| | LFKAKLIKAILPLYIEYIYEGEKLENAKKALKMYNRFTSRLSNFWQARA |
| | NIFTDDEISTGSPYRLVNDNFTIFRINNSIYTKNKPFIEEDILEFEKKLKSK |
| | KIIKDFESVDDYFTVNAFNKLCTQNGIDKYNSILGGFTTKEREKVKGLN |
| | ELFNLAQQSINKGKKGEYRKNIRLGKLTKLKKQILAISDSTSFLIEQIEDD |
| | QDLYNKIKDFFELLLKEEIENENIFTQYANLQKLIEQADLSKIYINAKHLN |
| | KISHQVTGKWDSLNKGIALLLENININEESKEKSEVISNGQTKDISSEAY |
| | KRYLQIQSEEKDIERLRTQIYFSLEDLEKALDLVLIDENMDRSDKSILSYV |
| | QSPDLNVNFERDLTDLYSRIMKLEENNEKLLANHSAIDLIKEFLDLIMLR |
| | YSRWQILFCDSNYELDQTFYPIYDAVMEILSNIIRLYNLARNYLSRKPDR |
| | MKKKKINFNNPTLADGWSESKIPDNSSMLFIKDGMYYLGIIKNRAAYSE |
| | LLEAESLQSSEKKKSENSSYERMNYHFLPDAFRSIPKSSIAMKAVKEHFE |
| | INQKTADLLLDTDKFSKPLRITKEIFDMQYVDLHKNKKKYQVDYLRDT |
| | GDKKGYRKALNTWLNFCKDFISKYKGRNLFDYSKIKDADHYETVNEF |
| | YNDVDKYSYHIFFTSVAETTVEKFISEGKLYLFQLYNKDFSPHSTGKPNL |
| | HTIYWRALFSEENLTSKNIKLNGQAEIFFRPKQIETPFTHKKGSILVNRFD |
| | VNGNPIPINVYQEIKGFKNNVIKWDDLNKTTQEGLENDQYLYFESEFEII |
| | KDRRYTEDQLFFHVPISFNWDIGSNPKINDLATQYIVNSNDIHIIGIDRGE |
| | NHLIYYSVIDLQGAIVEQGSLNTITEYTENKFLNNKTNNLRKIPYKDILQ |
| | QREDERADARIKWHAIDKIKDLKDGYLGQIVHFLAKLIIKYNAIVILEDL |
| | NYGFKRGRFKVERQVYQKFEMALMKKLNVLVFKDYDIDEIGGPLKPW |
| | QLTRPIDSYERMGRQNGILFYVPAAYTSAVDPVTGFANLFYLNNVKNSE |
| | KFHFFSKFESIKYHSDQDMFSFAFDYNNFGTTTRINDLSKSKWQVFTNH |
| | ERSVWNNKEKNYVTQNLTDLIKKLLRTYNIEFKNNQNVLDSILKIENNT |
| | DKENFARELFRLFRLTIQLRNTTVNENNTEITENELDYIISPVKDKNGNFF |
| | DSRDELKNLPDNGDANGAYNIARKGLLYIEQLQESIKTGKLPTLSISTLD |
| | WFNYIMK* |
| SEQ ID | MNKGGYVIMEKMTEKNRWENQFRITKTIKEEIIPTGYTKVNLQRVNML |
| NO: 233 | KREMERNEDLKKMKEICDEYYRNMIDVSLRLEQVRTLGWESLIHKYR |
| | MLNKDEKEIKALEKEQEDLRKKISKGFGEKKAWTGEQFIKKILPQYLM |
| | DHYTGEELEEKLRIVKKFKGCTMFLSTFFKNRENIFTDKPIHTAVGHRIT |
| | SENAMLFAANINTYEKMESNVTLEIERLQREFWRRGINISEIFTDAYYVN |
| | VLTQKQIEAYNKICGDINQHMNEYCQKQKLKFSEFRMRELKKQILAVV |
| | GEHFEIPEKIESTKEVYRELNEYYESLKELHGQFEELKSVQLKYSQIYVQ |
| | KKGYDRISRYIGGQWDLIQECMKKDCASGMKGTKKNHDAKIEEEVAK |
| | VKYQSIEHIQKLVCTYEEDRGHKVTDYVDEFIVSVCDLLGADHIITRDG |
| | ERIELPLQYEPGTDLLKNDTINQRRLSDIKTILDWHMDMLEWLKTFLVN |
| | DLVIKDEEFYMAIEELNERMQCVISVYNRIRNYVTQKGYEPEKIRICFDK |
| | GTILTGWTTGDNYQYSGFLLMRNDKYYLGIINTNEKSVRKILDGNEECK |
| | DENDYIRVGYHLINDASKQLPRIFVMPKAGKKSEILMKDEQCDYIWDG |
| | YCHNKHNESKEFMRELIDYYKRSIMNYDKWEGYCFKFSSTESYDNMQ |
| | DFYKEVREQSYNISFSYINENVLEQLDKDGKIYLFQVYNKDFAAGSTGT |
| | PNLHTMYLQNLFSSQNLELKRLRLGGNAELFYRPGTEKDVTHRKGSILV |
| | DRTYVREEKDGIEVRDTVPEKEYLEIYRYLNGKQKGDLSESAKQWLDK |
| | VHYREAPCDIIKDKRYAQEKYFLHFSVEINPNAEGQTALNDNVRRWLSE |
| | EEDIHVIGIDRGERNLIYVSLMDGKGRIKDQKSYNIVNSGNKEPVDYLA |
| | KLKVREKERDEARRNWKAIGKIKDIKTGYLSYVVHEIVEMAVREKAIIV |
| | MEDLNYGFKRGRFKVERQVYQKFEEMLINKLNYVVDKQLSVDEPGGL |
| | LRGYQLAFIPKDKKSSMRQNGIVFYVPAGYTSKIDPTTGFVNIFKFPQFG |
| | KGDDDGNGKDYDKIRAFFGKFDEIRYECDEKVTADNTREVKERYRFDF |
| | DYSKFETHLVHMKKTKWTVYAEGERIKRKKVGNYWTSEVISDIALRMS |
| | NTLNIAGIEYKDGHNLVNEICALRGKQAGIILNELLEIVRLTVQLRNSTTE |
| | GDVDERDEIISPVLNEKYGCFYHSTEYKQQNGDVLPKDADANGAYCIG |
| | LKGIYEIRQIKNKWKEDMTKGEGKALNEGMRISHDQWFEFIQNMNKGE |
| | * |
| SEQ ID | MNNPRGAFGGFTNLYSLSKTLRFELKPYLEIPEGEKGKLFGDDKEYYKN |
| NO: 234 | CKTYTEYYLKKANKEYYDNEKVKNTDLQLVNFLHDERIEDAYQVLKP |
| | VFDTLHEEFITDSLESAEAKKIDFGNYYGLYEKQKSEQNKDEKKKIDKP |
| | LETERGKLRKAFTPIYEAEGKNLKNKAGKEKKDKDILKESGFKVLIEAG |
| | ILKYIKNNIDEFADKKLKNNEGKEITKKDIETALGAENIEGIFDGFFTYFS |
| | GFNQNRENYYSTEEKATAVASRIVDENLSKFCDNILLYRKNENDYLKIFN |
| | FLKNKGKDLKLKNSKFGKENEPEFIPAYDMKNDEKSFSVADFVNCLSQG |
| | EIEKYNAKIANANYLINLYNQNKDGNSSKLSMFKILYKQIGCGEKKDFIK |
| | TIKDNAELKQILEKACEAGKKYFIRGKSEDGGVSNIFDFTDYIQSHENYK |
| | GVYWSDKAINTISGKYFANWDTLKNKLGDAKVFNKNTGEDKADVKY |
| | KVPQAVMLSELFAVLDDNAGEDWREKGIFFKASLFEGDQNKSEIIKNAN |
| | RPSQALLKMICDDMESLAKNFIDSGDKILKISDRDYQKDENKQKIKNWL |
| | DNALWINQILKYFKVKANKIKGDSIDARIDSGLDMLVFSSDNPAEDYDM |
| | IRNYLTQKPQDEINKLKLNFENSSLAGGWDENKEKDNSCIILKDEQDKQ |
| | YLAVMKYENTKVFEQKNSQLYIADNAAWKKMIYKLVPGASKTLPKVFF |
| | SKKWTANRPTPSDIVEIYQKGSFKKENVDFNDKKEKDESRKEKNREKII |
| | AELQKTCWMDIRYNIDGKIESAKYVNKEKLAKLIDFYKENLKKYPSEEE |
| | SWDRLFAFGFSDTKSYKSIDQFYIEVDKQGYKLEFVTINKARLDEYVRD |
| | GKIYLFEIRSRDNNLVNGEEKTSAKNLQTIYWNAAFGGDDNKPKLNGE |
| | AEIFYRPAIAENKLNKKKDKNGKEIIDGYRFSKEKFIFHCPITLNFCLKET |
| | KINDKLNAALAKPENGQGVYFLGIDRGEKHLAYYSLVNQKGEILEQGT |
| | LNLPFLDKNGKSRSIKVEKKSFEKDSNGGIIKDKDGNDKIKIEFVECWN |
| | YNDLLDARAGDRDYARKNWTTIGTIKELKDGYISQVVRKIVDLSIYKNT |
| | ETKEFREMPAFIVLEDLNIGFKRGRQKIEKQVYQKLELALAKKLNFLVD |
| | KKADIGEIGSVTKAIQLTPPVNNFGDMENRKQFGNMLYIRADYTSQTDP |
| | ATGWRKSIYLKSGSESNVKEQIEKSFFDIRYESGDYCFEYRDRHGKMW |
| | QLYSSKNGVSLDRFHGERNNSKNVWESEKQPLNEMLDILFDEKRFDKS |
| | KSLYEQMFKGGVALTRLPKEINKKDKPAWESLRFVIILIQQIRNTGKNGD |
| | DRNGDFIQSPVRDEKTGEHFDSRIYLDKEQKGEKADLPTSGDANGAYNI |
| | ARKGIVVAEHIKRGFDKLYISDEEWDTWLAGDEIWDKWLKENRESLTK |
| | TRK* |
| SEQ ID | MNYKTGLEDFIGKESLSKTLRNALIPTESTKIHMEEMGVIRDDELRAEK |
| NO: 235 | QQELKEIMDDYYRTFIEEKLGQIQGIQWNSLFQKMEETMEDISVRKDLD |
| | KIQNEKRKEICCYFTSDKRFKDLFNAKLITDILPNFIKDNKEYTEEEKAE |
| | KEQTRVLFQRFATAFTNYFNQRRNNFSEDNISTAISFRIVNENSEIHLQNM |
| | RAFQRIEQQYPEEVCGMEEEYKDMLQEWQMKHIYSVDFYDRELTQPGI |
| | EYYNGICGKINEHMNQFCQKNRINKNDFRMKKLHKQILCKKSSYYEIPF |
| | RFESDQEVYDALNEFIKTMKKKEIIRRCVHLGQECDDYDLGKIYISSNK |
| | YEQISNALYGSWDTIRKCIKEEYMDALPGKGEKKEEKAEAAAKKEEYR |
| | SIADIDKIISLYGSEMDRTISAKKCITEICDMAGQISIDPLVCNSDIKLLQN |
| | KEKTTEIKTILDSFLHVYQWGQTFIVSDIIEKDSYFYSELEDVLEDFEGIT |
| | TLYNHVRSYVTQKPYSTVKFKLHFGSPTLANGWSQSKEYDNNAILLMR |
| | DQKFYLGIFNVRNKPDKQIIKGHEKEEKGDYKKMIYNLLPGPSKMLPK |
| | VFITSRSGQETYKPSKHILDGYNEKRHIKSSPKFDLGYCWDLIDYYKECI |
| | HKHPDWKNYDFHFSDTKDYEDISGFYREVEMQGYQIKWTYISADEIQK |
| | LDEKGQIFLFQIYNKDFSVHSTGKDNLHTMYLKNLFSEENLKDIVLKLN |
| | GEAELFFRKASIKTPIVHKKGSVLVNRSYTQTVGNKEIRVSIPEEYYTEIY |
| | NYLNHIGKGKLSSEAQRYLDEGKIKSFTATKDIVKNYRYCCDHYFLHLPI |
| | TINFKAKSDVAVNERTLAYIAKKEDIHIIGIDRGERNLLYISVVDVHGNIR |
| | EQRSFNIVNGYDYQQKLKDREKSRDAARKNWEEIEKIKELKEGYLSMV |
| | IHYIAQLVVKYNAVVAMEDLNYGFKTGRFKVERQVYQKFETMLIEKLH |
| | YLVFKDREVCEEGGVLRGYQLTYIPESLKKVGKQCGFIFYVPAGYTSKI |
| | DPTTGFVNLFSFKNLTNRESRQDFVGKFDEIRYDRDKKMFEFSFDYNNY |
| | IKKGTILASTKWKVYTNGTRLKKIVVNGKYTSQSMEVELTDAMEKML |
| | QRAGIEYHDGKDLKGQIVEKGIEAEIIDIFRLTVQMRNSRSESEDREYDR |
| | LISPVLNDKGEFFDTATADKTLPQDADANGAYCIALKGLYEVKQIKENW |
| | KENEQFPRNKLVQDNKTWFDFMQKKRYL* |
| SEQ ID | MRISKTLSLRIVRPFYTPEVEAGIKAEKDKREAQGQTRSLDAKFFNELK |
| NO: 236 | KKHSEIILSSEFYSLLSEVQRQLTSIYNHAMSNLYHKIIVEGEKTSTSKAL |
| | SNIGYDECKAIFPSYMALGLRQKIQSNFRRRDLKNFRMAVPTAKSDKFPI |
| | PIYRQVDGSKGGFKISENDGKDFIVELPLVDYVAEEVKTAKGRFTKINIS |
| | KPPKIKNIPVILSTLRRRQSGQWFSDDGTNAEIRRVISGEYKVSWIEIVRR |
| | TRFGKHDDWFVNMVIKYDKPEEGLDSKVVGGIDVGVSSPLVCALNNSL |
| | DRYFVKSSDIIAFNKRAMARRRTLLRQNKYKRSGHGSKNKLEPITVLTE |
| | KNERFKKSIMQRWAKEVAEFFRGKGASVVRMEELSGLKEKDNFFSSYL |
| | RMYWNYGQLQQIIENKLKEYGIKVNYVSPKDTSKKCHSCTHINEFFTFE |
| | YRQKNNFPLFKCEKCGVECSADYNAAKNMAIA |
| SEQ ID | MRTMVTFEDFTKQYQVSKTLRFELIPQGKTLENMKRDGIISVDRQRNED |
| NO: 237 | YQKAKGILDKLYKYILDFTMETVVIDWEALATATEEFRKSKDKKTYEKV |
| | QSKIRTALLEHVKKQKVGTEDLFKGMFSSKIITGEVLAAFPEIRLSDEEN |
| | LILEKFKDFTTYFTGFFENRKNVFTDEALSTSFTYRLVNDNFIKFFDNCIV |
| | FKNVVNISPHMAKSLETCASDLGIFPGVSLEEVFSISFYNRLLTQTGIDQF |
| | NOLLGGISGKEGEHKQQGLNEIINLAMQQNLEVKEVLKNKAHRFTPLF |
| | KQILSDRSTMSFIPDAFADDDEVLSAVDAYRKYLSEKNIGDRAFQLISDM |
| | EAYSPELMRIGGKYVSVLSQLLFYSWSEIRDGVKAYKESLITGKKTKKE |
| | LENIDKEIKYGVTLQEIKEALPKKDIYEEVKKYAMSVVKDYHAGLAEPL |
| | PEKIETDDERASIKHIMDSMLGLYRFLEYFSHDSIEDTDPVFGECLDTILD |
| | DMNETVPLYNKVRNFSTRKVYSTEKFKLNFNNSSLANGWDKNKEQAN |
| | GAILLRKEGEYFLGIFNSKNKPKLVSDGGAGIGYEKMIYKQFPDFKKML |
| | PKCTISLKDTKAHFQKSDEDFTLQTDKFEKSIVITKQIYDLGTQTVNGKK |
| | KFQVDYPRLTGDMEGYRAALKEWIDFGKEFIQAYTSTAIYDTSLFRDSS |
| | DYPDLPSFYKDVDNICYKLTFEWIPDAVIDDCIDDGSLYLFKLHNKDFSS |
| | GSIGKPNLHTLYWKALFEEENLSDVVVKLNGQAELFYRPKSLTRPVVHE |
| | EGEVIINKTTSTGLPVPDDVYVELSKFVRNGKKGNLTDKAKNWLDKVT |
| | VRKMPHAITKDRRFTVDKFFFHVPITLNYKADSSPYRFNDFVRQYIKDC |
| | SDVKIIGIDRGERNLIYAVVIDGKGNIIEQRSFNTVGTYNYQEKLEQKEKE |
| | RQTARQDWATVTKIKDLKKGYLSAVVHELSKMIVKYKAIVALENLNVG |
| | FKRMRGGIAERSVYQQFEKALIDKLNYLVFKDEEQSGYGGVLNAYQLT |
| | DKFESFSKMGQQTGFLFYVPAAYTSKIDPLTGFINPFSWKHVKNREDRR |
| | NFLNLFSKLYYDVNTHDFVLAYHHSNKDSKYTIKGNWEIADWDILIQEN |
| | KEVFGKTGTPYCVGKRIVYMDDSTTGHNRMCAYYPHTELKKLLSEYGI |
| | EYTSGQDLLKIIQEFDDDKLVKGLFYIIKAALQMRNSNSETGEDYISSPIE |
| | GRPGICFDSRAEADTLPYDADANGAFHIAMKGLLLTERIRNDDKLAISN |
| | EEWLNYIQEMRG* |
| SEQ ID | MSKLEKFTNCYSLSKTLRFKAIPVGKTQENIDNKRLLVEDEKRAEDYKG |
| NO: 238 | VKKLLDRYYLSFINDVLHSIKLKNLNNYISLFRKKTRTEKENKELENLEI |
| | NLRKEIAKAFKGNEGYKSLFKKDIIETILPEFLDDKDEIALVNSFNGFTTA |
| | FTGFFDNRENMFSEEAKSTSIAFRCINENLTRYISNMDIFEKVDAIFDKHE |
| | VQEIKEKILNSDYDVEDFFEGEFFNFVLTQEGIDVYNAIIGGFVTESGEKI |
| | KGLNEYINLYNQKTKQKLPKFKPLYKQVLSDRESLSFYGEGYTSDEEVL |
| | EVFRNTLNKNSEIFSSIKKLEKLFKNFDEYSSAGIFVKNGPAISTISKDIFG |
| | EWNVIRDKWNAEYDDIHLKKKAVVTEKYEDDRRKSFKKIGSFSLEQLQ |
| | EYADADLSVVEKLKEIIIQKVDEIYKVYGSSEKLFDADFVLEKSLKKND |
| | AVVAIMKDLLDSVKSFENYIKAFFGEGKETNRDESFYGDFVLAYDILLK |
| | VDHIYDAIRNYVTQKPYSKDKFKLYFQNPQFMGGWDKDKETDYRATIL |
| | RYGSKYYLAIMDKKYAKCLQKIDKDDVNGNYEKINYKLLPGPNKMLP |
| | KVFFSKKWMAYYNPSEDIQKIYKNGTFKKGDMFNLNDCHKLIDFFKDS |
| | ISRYPKWSNAYDFNFSETEKYKDIAGFYREVEEQGYKVSFESASKKEVD |
| | KLVEEGKLYMFQIYNKDFSDKSHGTPNLHTMYFKLLFDENNHGQIRLS |
| | GGAELFMRRASLKKEELVVHPANSPIANKNPDNPKKTTTLSYDVYKDK |
| | RFSEDQYELHIPIAINKCPKNIFKINTEVRVLLKHDDNPYVIGIDRGERNL |
| | LYIVVVDGKGNIVEQYSLNEIINNFNGIRIKTDYHSLLDKKEKERFEARQ |
| | NWTSIENIKELKAGYISQVVHKICELVEKYDAVIALEDLNSGFKNSRVKV |
| | EKQVYQKFEKMLIDKLNYMVDKKSNPCATGGALKGYQITNKFESFKS |
| | MSTQNGFIFYIPAWLTSKIDPSTGFVNLLKTKYTSIADSKKFISSFDRIMY |
| | VPEEDLFEFALDYKNFSRTDADYIKKWKLYSYGNRIRIFRNPKKNNVFD |
| | WEEVCLTSAYKELFNKYGINYQQGDIRALLCEQSDKAFYSSFMALMSL |
| | MLQMRNSITGRTDVDFLISPVKNSDGIFYDSRNYEAQENAILPKNADAN |
| | GAYNIARKVLWAIGQFKKAEDEKLDKVKIAISNKEWLEYAQTSVKH |
| SEQ ID | MTNFDNFTKKYVNSKTIRLEAIPVGKTLKNIEKMGFIAADRQRDEDYQ |
| NO: 239 | KAKSVIDHIYKAFMDDCLKDLFLDWDPLYEAVVACWRERSPEGRQALQ |
| | IMQADYRKKIADRFRNHELYGSLFTKKIFDGSVAQRLPDLEQSAEEKSL |
| | LSNFNKFTSYFRDFFDKRKRLFSDDEKHSAIAYRLINENFLKFVANCEAF |
| | RRMTERVPELREKLQNTGSLQVYNGLALDEVFSADFYNQLIVQKQIDLY |
| | NQLIGGIAGEPGTPNIQGLNATINLALQGDSSLHEKLAGIPHRFNPLYKQI |
| | LSDVSTLSFVPSAFQSDGEMLAAVRGFKVQLESGRVLQNVRRLENGLET |
| | EADLSRVYVNNSKLAAFSSMFFGRWNLCSDALFAWKKGKQKKITNKK |
| | LTEIKKWLKNSDIAIAEIQEAFGEDFPRGKINEKIQAQADALHSQLALPIP |
| | ENLKALCAKDGLKSMLDTVLGLYRMLQWFIVGDDNEKDSDFYFGLGK |
| | ILGSLDPVLVLYNRVRNYITKKPYSLTKFRLNFDNSQLLNGWDENNLDT |
| | NCASIFIKDGKYYLGISNKNNRPQFDTVATSGKSGYQRMVYKQFANWG |
| | RDLPHSTTQMKKVKKHFSASDADYVLDGDKFIRPLIITKEIFDLNNVKF |
| | NGKKKLQVDYLRNTGDREGYTHALHTWINFAKDFCACYKSTSIYDISC |
| | LRPTDQYDNLMDFYADLGNLSHRIVWQTIPEEAIDNYVEQGQLFLFQLY |
| | NKDFAPGADGKPNLHTLYWKAVFNPENLEDVVVKLNGKAELFYRPRS |
| | NMDVVRHKVGEKLVNRKLKNGLTLPSRLHEEIYRYVNGTLNKDLSAD |
| | ARSVLPLAVVRDVQHEIIKDRRFTADKFFFHASLTFNFKSSDKPVGFNED |
| | VREYLREHPDTYVVGVDRGERNLIYIVVIDPQGNIVEQRSFNMINGIDY |
| | WSLLDQKEKERVEAKQAWETVGKIKDLKCGYLSFLIHEITKIIIKYHAVV |
| | ILENLSLGFKRVRTGIAEKAVYQQFERMLVTKLGYVVFKDRAGKAPGG |
| | VLNAYQLTDNTRTAENTGIQNGFLFYVPAAFTSRVDPATGFFDFYDWGK |
| | IKTATDKKNFIAGFNSVRYERSTGDFIVHVGAKNLAVRRVAEDVRTEWD |
| | IVIEANVRKMGIDGNSYISGKRIRYRSGEQGHGQYENHLPCQELIRALQ |
| | QYGIQYETGKDILPAILQQDDAKLTDTVFDVFRLALQMRNTSAETGEDY |
| | FNSVVRDRSGRCFDTRRAEAAMPKEADANDAYHIALKGLFVLEKLRKG |
| | ESIGIKNTEWLRYVQQRHS* |
| SEQ ID | MTPIFCNFVVYQIMLFNNNININVKTMNKKHLSDFTNLFPVSKTLRFRL |
| NO: 240 | EPQGKTMENIVKAQTIETDEERSHDYEKTKEYIDDYHRQFIDDTLDKFA |
| | FKVESTGNNDSLQDYLDAYLSANDNRTKQTEEIQTNLRKAIVSAFKMQ |
| | PQFNLLFKKEMVKHLLPQFVDTDDKKRIVAKFNDFTTYFTGFFTNREN |
| | MYSDEAKSTSIAYRIVNQNLIKFVENMLTFKSHILPILPQEQLATLYDDFK |
| | EYLNVASIAEMFELDHFSIVLTQRQIEVYNSVIGGRKDENNKQIKPGLNQ |
| | YINQHNQAVKDKSARLPLLKPLFNQILSEKAGVSFLPKQFKSASEVVKS |
| | LNEAYAELSPVLAAIQDVVTNITDYDCNGIFIKNDLGLTDIAQRFYGNYD |
| | AVKRGLRNQYELETPMHNGQKAEKYEEQVAKHLKSIESVSLAQINQVV |
| | TDGGDICDYFKAFGATDDGDIQRENLLASINNAHTAISPVLNKENANDN |
| | ELRKNTMLIKDLLDAIKRLQWFAKPLLGAGDETNKDQVFYGKFEPLYN |
| | QLDETISPLYDKVRSYLTKKPYSLDKFKINFEKSNLLGGWDPGADRKYQ |
| | YNAVILRKDNDFYLGIMRDEATSKRKCIQVLDCNDEGLDENFEKVEYK |
| | QIKPSQNMPRCAFAKKECEENADIMELKRKKNAKSYNTNKDDKNALIR |
| | HYQRYLDRTYPEFGFVYKDADEYDTVKAFTDSMDSQDYKLSFLQVSET |
| | GLNKLVDEGDLYLFKITNKDFSSYAKGRPNLHTIYWRMLFDPKNLANV |
| | VYKLEGKAEVFFRRKSLASTTTHKAKQAIKNKSRYNEAVKPQSTFDYDI |
| | IKDRRFTADKFEFHVPIKMNFKAAGWNSTRLTNEVREFIKSQGVRHIIGI |
| | DRGERHLLYLTMIDMDGNIVKQCSLNAPAQDNARASEVDYHQLLDSKE |
| | ADRLAARRNWGTIENIKELKQGYLSQVVHLLATMMVDNDAILVLENLN |
| | AGFMRGRQKVEKSVYQKFEKMLIDKLNYIVDKGQSPDKPTGALHAVQ |
| | LTGLYSDFNKSNMKRANVRQCGFVFYIPAWNTSKIDPVTGFVNLFDTHL |
| | SSMGEIKAFFSKFDSIRYNQDKGWFEFKFDYSRFTTRAEGCRTQWTVCT |
| | YGERIWTHRSKNQNNQFVNDTVNVTQQMLQLLQDCGIDPNGNLKEAI |
| | ANIDSKKSLETLLHLFKLTVQMRNSVTGSEVDYMISPVADERGHFFDSR |
| | ESDEHLPANADANGAFNIARKGLMVVRQIMATDDVSKIKFAVSNKDWL |
| | RFAQHIDD* |
| SEQ ID | VKISKTLSLRIIRPYYTPEVESAIKAEKDKREAQGQTRNLDAKFFNELKK |
| NO: 241 | KHPQIILSGEFYSLLFEMQRQLTSIYNRAMSSLYHKIIVEGEKTSTSKALS |
| | DIGYDECKSVFPSYIALGLRQKIQSNFRRKELKGFRMAVPTAKSDKFPIPI |
| | YKQVDDGKGGFKISENKEGDFIVELPLVEYTAEDVKTAKGKFTKINISKP |
| | PKIKNIPVILSTLRRKQSGQWFSDEGTNAEIRRVISGEYKVSWIEVVRRTR |
| | FGKHDDWFLNIVIKYDKTEDGLDPEVVGGIDVGVSTPLVCAVNNSLDR |
| | YFVKSSDIIAFKKRAMARRRTLLRQNRFKRSGHGSKSKLEPITILTEKNE |
| | RFKKSIMQRWAKEVAEFFKGERASVVQMEELSGLKEKDNFFGSYLRMY |
| | WNYGQLQQIIENKLKEYGIKVNYVSPKDTSKKCHSCGYINEFFTFEFRQ |
| | KNNFPLFKCKKCGVECNADYNAAKNIAIA |
| SEQ ID | VKLPILKPLHKQILSEEYSTSFKIKAFENDNEVLKAIDTFWNEHIEKSIHP |
| NO: 242 | VTGNKFNILSKIENLCDQLQKYKDKDLEKLFIERKNLSTVSHQVYGQW |
| | NIIRDALRMHLEMNNKNIKEKDIDKYLDNDAFSWKEIKDSIKIYKEHVE |
| | DAKELNENGIIKYFSAMSINEEDDEKEYSISLIKNINEKYNNVKSILQEDR |
| | TGKSDLHQDKEKVGIIKEFLDSLKQLQWFLRLLYVTVPLDEKDYEFYNE |
| | LEVYYEALLPLNSLYNKVRNYMTRKPYSVEKFKLNFNSPTLLDGWDKN |
| | KETANLSIILRKNGKYYLGIMNKENNTIFEYYPGTKSNDYYEKMIYKLL |
| | PGPNKMLPKVFFSKKGLEYYNPPKEILNIYEKGEFKKDKSGNFKKESLH |
| | TLIDFYKEAIAKNEDWEVFNFKFKNTKEYEDISQFYRDVEEQGYLITFE |
| | KVDANYVDKLVKEGKLYLFQIYNKDFSENKKSKGNPNLHTIYWKGLYD |
| | SENLKNVVYKLNGEAEVFYRKKSIDYPEEIYNHGHHKEELLGKFNYPII |
| | KDRRYTQDKFLFHVPITMNFISKEEKRVNQLACEYLSATKEDVHIIGIDR |
| | GERHLLYLSLIDKEGNIKKQLSLNTIKNENYDKEIDYRVKLDEKEKKRD |
| | EARKNWDVIENIKELKEGYMSQVIHIIAKMMVEEKAILIMEDLNIGFKR |
| | GRFKVEKQVYQKFEKMLIDKLNYLVFKNKNPLEPGGSLNAYQLTSKFD |
| | SFKKLGKQSGFIFYVPSAYTSKIDPTTGFYNFIQVDVPNLEKGKEFFSKFE |
| | KIIYNTKEDYFEFHCKYGKFVSEPKNKDNDRKTKESLTYYNAIKDTVW |
| | VVCSTNHERYKIVRNKAGYYESHPVDVTKNLKDIFSQANINYNEGKDI |
| | KPIIIESNNAKLLKSIAEQLKLILAMRYNNGKHGDDEKDYILSPVKNKQG |
| | KFFCTLDGNQTLPINADANGAYNIALKGLLLIEKIKKQQGKIKDLYISNL |
| | EWFMFMMSR |

Effector proteins disclosed herein may function as an endonuclease that catalyzes cleavage at a specific position (e.g., at a specific nucleotide within a nucleic acid sequence) in a target nucleic acid. The target nucleic acid may be single stranded RNA (ssRNA), double stranded DNA (dsDNA) or single-stranded DNA (ssDNA). In some instances, the target nucleic acid is single-stranded DNA. In some instances, the target nucleic acid is single-stranded RNA. The effector proteins may provide cis cleavage activity, trans cleavage activity, nickase activity, or a combination thereof. Cis cleavage activity is cleavage of a target nucleic acid that is hybridized to a guide nucleic acid (e.g., a dual gRNA or a sgRNA), wherein cleavage occurs within or directly adjacent to the region of the target nucleic acid that is hybridized to guide nucleic acid. Trans cleavage activity (also referred to as transcollateral cleavage) is cleavage of ssDNA or ssRNA that is near, but not hybridized to the guide nucleic acid. Trans cleavage activity is triggered by the hybridization of guide nucleic acid to the target nucleic acid. Nickase activity is a selective cleavage of one strand of a dsDNA.

Effector proteins of the present disclosure, dimers thereof, and multimeric complexes thereof may cleave or nick a target nucleic acid within or near a protospacer adjacent motif (PAM) sequence of the target nucleic acid. In some instances, cleavage occurs within 1, 2, 3, 4, 5, 6, 7, 8, 9 or 10 nucleosides of a 5′ or 3′ terminus of a PAM sequence. A target nucleic acid may comprise a PAM sequence adjacent to a sequence that is complementary to a guide nucleic acid spacer region.

### Engineered Proteins

In some instances, effector proteins disclosed herein are engineered proteins. Engineered proteins are not identical to a naturally-occurring protein. Engineered proteins may provide enhanced nuclease or nickase activity as compared to a naturally occurring nuclease or nickase. An engineered protein may comprise a modified form of a wild type counterpart protein.

In some instances, effector proteins comprise at least one amino acid change (e.g., deletion, insertion, or substitution) that reduces the nucleic acid-cleaving activity of the effector protein relative to the wild type counterpart. For example, a nuclease domain (e.g., RuvC domain) of an effector protein may be deleted or mutated relative to a wild type counterpart effector protein so that it is no longer functional or comprises reduced nuclease activity. The effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered proteins may have no substantial nucleic acid-cleaving activity. Engineered proteins may be enzymatically inactive or “dead,” that is it may bind to a nucleic acid but not cleave it. An enzymatically inactive protein may comprise an enzymatically inactive domain (e.g. inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with a guide nucleic acid to activate or repress transcription of a target nucleic acid sequence. In some instances, the enzymatically inactive protein is fused with a protein comprising recombinase activity.

In some instances, effector proteins comprise at least one amino acid change (e.g., deletion, insertion, or substitution) that increases the nucleic acid-cleaving activity of the effector protein relative to the wild type counterpart. The effector protein may provide at least about 20%, at least about 30%, at least about 40%, at least about 50% at least about 60%, at least about 70%, at least about 80%, at least about 90%, or about 100% more nucleic acid-cleaving activity relative to that of the wild-type counterpart. The effector protein may provide at least about 2 fold, at least about 3 fold, at least about 4 fold, at least about 5 fold, at least about 6 fold, at least about 7 fold, at least about 8 fold, at least about 9 fold or at least about 10 fold more nucleic acid-cleaving activity relative to that of the wild-type counterpart.

### Fusion Proteins

In some instances, an effector protein is a fusion protein, wherein the fusion protein comprises a Cas effector protein and a fusion partner protein. A fusion partner protein is also simply referred to herein as a fusion partner. The fusion partner may comprise a protein or a functional domain thereof. Non-limiting examples of fusion partners include cell surface receptor proteins, intracellular signaling proteins, transcription factors, or functional domains thereof. The fusion partner may comprise a signaling peptide, e.g., a nuclear localization signal (NLS).

In some instances, the fusion partner modulates transcription (e.g., inhibits transcription, increases transcription) of a target nucleic acid. In some instances, the fusion partner is a protein (or a domain from a protein) that inhibits transcription of a target nucleic acid, also referred to as a transcriptional repressor. Transcriptional repressors may inhibit transcription via recruitment of transcription inhibitor proteins, modification of target DNA such as methylation, recruitment of a DNA modifier, modulation of histones associated with target DNA, recruitment of a histone modifier such as those that modify acetylation and/or methylation of histones, or a combination thereof. In some instances, the fusion partner is a protein (or a domain from a protein) that increases transcription of a target nucleic acid, also referred to as a transcription activator. Transcriptional activators may promote transcription via recruitment of transcription activator proteins, modification of target DNA such as demethylation, recruitment of a DNA modifier, modulation of histones associated with target DNA, recruitment of a histone modifier such as those that modify acetylation and/or methylation of histones, or a combination thereof.

In some instances, the fusion protein is a base editor. In general, a base editor comprises a deaminase. In some instances, a fusion protein that comprises a deaminase and a Cas effector protein changes a nucleobase to a different nucleobase, e.g., cytosine to thymine or guanine to adenine.

In some instances, fusion partners provide enzymatic activity that modifies a target nucleic acid. Such enzymatic activities include, but are not limited to, histone acetyltransferase activity, histone deacetylase activity, nuclease activity, methyltransferase activity, demethylase activity, DNA repair activity, DNA damage activity, deamination activity, dismutase activity, alkylation activity, depurination activity, oxidation activity, kinase activity, phosphatase activity, pyrimidine dimer forming activity, integrase activity, transposase activity, recombinase activity, polymerase activity, ligase activity, helicase activity, photolyase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity, and glycosylase activity. In some instances, the fusion partner comprises an RNA splicing factor.

### Multimeric Complexes

In some instances, an effector protein may form a multimeric complex with another protein. In general, a multimeric complex comprises multiple programmable nucleases that non-covalently interact with one another. A multimeric complex may comprise enhanced activity relative to the activity of any one of its programmable nucleases alone. For example, a multimeric complex comprising two programmable nucleases may comprise greater nucleic acid binding affinity, cis-cleavage activity, and/or transcollateral cleavage activity than that of either of the programmable nucleases provided in monomeric form. A multimeric complex may have an affinity for a target region of a target nucleic acid and is capable of catalytic activity (e.g., cleaving, nicking or modifying the nucleic acid) at or near the target region. Multimeric complexes may be activated when complexed with a guide nucleic acid. Multimeric complexes may be activated when complexed with a guide nucleic acid and a target nucleic acid. In some instances, the multimeric complex cleaves the target nucleic acid. In some instances, the multimeric complex nicks the target nucleic acid.

In some instances, the multimeric complex is a dimer comprising two programmable nucleases of identical amino acid sequences. In some instances, the multimeric complex comprises a first programmable nuclease and a second programmable nuclease, wherein the amino acid sequence of the first programmable nuclease is at least 90%, at least 92%, at least 94%, at least 96%, at least 98% identical, or at least 99% identical to the amino acid sequence of the second programmable nuclease. In some instances, the multimeric complex is a heterodimeric complex comprising at least two programmable nucleases of different amino acid sequences. In some instances, the multimeric complex is a heterodimeric complex comprising a first programmable nuclease and a second programmable nuclease, wherein the amino acid sequence of the first programmable nuclease is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second programmable nuclease.

In some instances, a multimeric complex comprises at least two programmable nucleases. In some instances, a multimeric complex comprises more than two programmable nucleases. In some instances, multimeric complexes comprise at least one Type V CRISPR/Cas protein, or a fusion protein thereof. In some instances, a multimeric complex comprises two, three or four Cas14 proteins.

### Thermostable Programmable Nucleases

Described herein are various embodiments of thermostable programmable nucleases. In some embodiments, a programmable nuclease is referred to as a programmable nuclease. A programmable nuclease may be thermostable. In some instances, known programmable nucleases (e.g., Cas12 nucleases) are relatively thermo-sensitive and only exhibit activity (e.g., cis and/or trans cleavage) sufficient to produce a detectable signal in a diagnostic assay at temperatures less than 40° C., and optimally at about 37° C. A thermostable protein may have enzymatic activity, stability, or folding comparable to those at 37° C. In some instances, the trans cleavage activity (e.g., the maximum trans cleavage rate as measured by fluorescent signal generation) of a programmable nuclease in a trans cleavage assay at 40° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of a programmable nuclease in a trans cleavage assay at 45° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of a programmable nuclease in a trans cleavage assay at 50° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of a programmable nuclease in a trans cleavage assay at 55° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of a programmable nuclease in a trans cleavage assay at 60° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of a programmable nuclease in a trans cleavage assay at 65° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of a programmable nuclease in a trans cleavage assay at 70° C., 75° C. 80° C., or more may be at least 50, at least 60%, at least 65%, at least 70%, at least 75%, at least 80%, at least 85%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

### Guide Nucleic Acids

Provided herein are compositions comprising one or more engineered guide nucleic acids. A guide nucleic acid can comprise a sequence that is reverse complementary to the sequence of a target nucleic acid. Guide nucleic acids are often referred to as a “guide RNA.” However, a guide nucleic acid may comprise deoxyribonucleotides. The term “guide RNA,” as well as crRNA and tracrRNA, includes guide nucleic acids comprising DNA bases, RNA bases, and modified nucleobases. In general, a guide nucleic acid is a nucleic acid molecule that binds to an effector protein (e.g., a Cas effector protein), thereby forming a ribonucleoprotein complex (RNP). In some instances, the engineered guide RNA imparts activity or sequence selectivity to the effector protein. In general, the engineered guide nucleic acid comprises a CRISPR RNA (crRNA) that is at least partially complementary to a target nucleic acid. In some instances, the engineered guide nucleic acid comprises a trans-activating crRNA (tracrRNA), at least a portion of which interacts with the effector protein. The tracrRNA may hybridize to a portion of the guide RNA that does not hybridize to the target nucleic acid. In some instances, the crRNA and tracrRNA are provided as a single guide nucleic acid, also referred to as a single guide RNA (sgRNA). In some instances, a crRNA and tracrRNA function as two separate, unlinked molecules.

The compositions of this disclosure can comprise a guide nucleic acid. The guide nucleic acid can bind to a single stranded target nucleic acid or portion thereof as described herein. For example, the guide nucleic acid can bind to a target nucleic acid such as nucleic acid from a virus or a bacterium or other agents responsible for a disease, or an amplicon thereof, as described herein. The guide nucleic acid can bind to a target nucleic acid such as a nucleic acid from a bacterium, a virus, a parasite, a protozoa, a fungus or other agents responsible for a disease, or an amplicon thereof, as described herein and further comprising a mutation, such as a single nucleotide polymorphism (SNP), which can confer resistance to a treatment, such as antibiotic treatment. The guide nucleic acid can bind to a target nucleic acid such as a nucleic acid, preferably DNA, from a cancer gene or gene associated with a genetic disorder, or an amplicon thereof, as described herein. The guide nucleic acid comprises a segment of nucleic acids that are reverse complementary to the target nucleic acid. Often the guide nucleic acid binds specifically to the target nucleic acid. The target nucleic acid can be a reversed transcribed RNA, DNA, DNA amplicon, or synthetic nucleic acids. The target nucleic acid can be a single-stranded DNA or DNA amplicon of a nucleic acid of interest.

A guide nucleic acid can comprise a sequence that is reverse complementary to the sequence of a target nucleic acid. A guide nucleic acid can be a crRNA. Sometimes, a guide nucleic acid comprises a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. In some cases, the segment of a guide nucleic acid that comprises a sequence that is reverse complementary to the target nucleic acid is 20 nucleotides in length. The segment of the guide nucleic acid that comprises a sequence that is reverse complementary to the target nucleic acid can have a length of at least 10, 11, 12, 13, 14, 15, 16, 17, 18, 19, 20, 21, 22, 23, 24, 25, 26, 27, 28, 29, or 30 nucleotides in length. In some instances, the segment of the guide nucleic acid that comprises a sequence that is reverse complementary to the target nucleic acid is 10, 11, 12, 13, 14, 15, 16, 17, 18, 19, 20, 21, 22, 23, 24, 25, 26, 27, 28, 29, or 30 nucleotides in length. In some cases, the segment of a guide nucleic acid that comprises a sequence that is reverse complementary to the target nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the segment of a guide nucleic acid that comprises a sequence that is reverse complementary to the target nucleic acid has a length of from about 10 nt to about 60 nt, from about 20 nt to about 50 nt, or from about 30 nt to about 40 nt. It is understood that the sequence of a polynucleotide need not be 100% complementary to that of its target nucleic acid to be specifically hybridizable or hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid. The guide nucleic acid can hybridize with a target nucleic acid.

The guide nucleic acid can be selected from a group of guide nucleic acids that have been tiled against the nucleic acid sequence of a strain of an infection or genomic locus of interest. The guide nucleic acid can be selected from a group of guide nucleic acids that have been tiled against the nucleic acid sequence interest, such as a strain of HPV 16 or HPV18. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances, the tiling of the guide nucleic acids is non-sequential. Often, a method for detecting a target nucleic acid comprises contacting a target nucleic acid to a pool of guide nucleic acids and a programmable nuclease, wherein a guide nucleic acid sequence of the pool of guide nucleic acids has a sequence selected from a group of tiled guide nucleic acid that correspond to nucleic acid sequence of a target nucleic acid; and assaying for a signal produce by cleavage of at least some reporter molecules of a population of reporter molecules. Pooling of guide nucleic acids can ensure broad spectrum identification, or broad coverage, of a target species within a single reaction. This can be particularly helpful in diseases or indications, like sepsis, that can be caused by multiple organisms.

### Catalytic Oligonucleotides

The present disclosure provides compositions and methods of use thereof comprising catalytic oligonucleotides. The catalytic oligonucleotide can comprise an RNA cleaving DNA enzyme. The catalytic oligonucleotide can comprise an RNA cleaving RNA enzyme. In some embodiments, a catalytic oligonucleotide comprises DNA. In some embodiments, a catalytic oligonucleotides comprises RNA. In some embodiments, a catalytic oligonucleotide comprises DNA and RNA. The catalytic oligonucleotide can have a catalytic activity. The catalytic activity can comprise binding to and subsequently cleaving a nucleic acid sequence, such as a nucleic acid sequence of a reporter molecule.

The catalytic oligonucleotide can be a deoxyribozymes, also called DNA enzymes, DNAzymes, or catalytic DNA DNAzyme. DNAzymes are DNA sequences (e.g., short sequences of DNA) which can form secondary structures that are capable of performing catalytic reactions, such as cleavage of a nucleic acid (e.g., RNA of a reporter molecule). In some examples, DNAzymes can be synthetic. In some cases, DNAzymes can be naturally-occuring. Some DNAzymes can be activated upon binding a co-factor. In some examples, a co-factor can be a small molecule co-factor. Some DNAzymes can be active without co-factors.

The catalytic oligonucleotide can be a ribozyme. Ribozymes are RNA sequences (e.g., short sequences of RNA) which can form secondary structures that are capable of performing catalytic reactions, such as cleavage of a nucleic acid (e.g., RNA of a reporter molecule). In some examples, ribozymes can be synthetic. In some cases, ribozymes can be naturally-occurring.

The catalytic oligonucleotide can be a multi-component nucleic acid enzyme, also referred to as MNAzymes. MNAzymes require an assembly facilitator for their assembly and catalytic activity. MNAzymes are composed of multiple part-enzymes, or partzymes, which self-assemble in the presence of one or more assembly facilitators to form secondary structures that are capable of performing catalytic reactions, such as cleavage of a nucleic acid (e.g., RNA of a reporter molecule).

The catalytic oligonucleotide can be an aptazyme. Aptazymes are catalytic oligonucleotides (e.g., DNAzymes, ribozymes, or MNAzymes) which have been linked with an aptamer domain to allosterically regulate the catalytic oligonucleotides such that their activity is dependent on the presence of the target analyte/ligand capable of binding to the aptamer domain.

In some embodiments, the compositions comprises two different catalytic oligonucleotides. For example, the composition comprises a first catalytic oligonucleotide and a second oligonucleotide. In some embodiments, the first catalytic oligonucleotide comprises an RNA cleaving DNA enzyme. The first catalytic oligonucleotide can comprise an RNA cleaving RNA enzyme. In some embodiments, a first catalytic oligonucleotide comprises DNA. In some embodiments, a first catalytic oligonucleotides comprises RNA. In some embodiments, a first catalytic oligonucleotide comprises DNA and RNA. The first catalytic oligonucleotide can have a catalytic activity. The catalytic activity of the first catalytic oligonucleotide can comprise binding to and subsequently cleaving a nucleic acid sequence, such as a nucleic acid sequence of a reporter molecule. In some embodiments, the second catalytic oligonucleotide comprises an RNA cleaving DNA enzyme. The second catalytic oligonucleotide can comprise an RNA cleaving RNA enzyme. In some embodiments, a second catalytic oligonucleotide comprises DNA. In some embodiments, a second catalytic oligonucleotide comprises RNA. In some embodiments, a second catalytic oligonucleotide comprises DNA and RNA. The second catalytic oligonucleotide can have a catalytic activity. The catalytic activity of the second catalytic oligonucleotide can comprise binding to and subsequently cleaving a nucleic acid sequence, such as a nucleic acid sequence of a blocker oligonucleotide (e.g., a first blocker oligonucleotide) bound to the first catalytic oligonucleotide.

A variety of catalytic oligonucleotides can be using for performing the methods of the present disclosure. Exemplary catalytic oligonucleotide sequences are provided in TABLE 6. An exemplary sequence that is not catalytically active, but can be used to generate a catalytically active version of DZ-precursor-1 by ligating into a circle (e.g., is a ligation splint) and which can function with DZ-beacon 1 is TCGTTGTAGCTAGCC (SEQ ID NO: 188). An exemplary sequence of a linear activated version of DZ-precursor-1 that does not require circularization and can function with DZ-beacon-1 is AATACAGGTAAGGCTAGCTACAACGACTAGCAGA (SEQ ID NO: 189; DZ-act-linear).

TABLE 6
Exemplary Catalytic Oligonucleotides

| Catalytic | |
|---|---|
| Oligo- | |
| nucleotide | Sequence (SEQ ID NO:) |
| DZ- | /5phos/TACAACGACTAGCArUrUrUrUrUCAGGTAAGGCTAGC (SEQ ID |
| precursor- | NO: 243) |
| 1 | |
| DZ-2 | GCCCAGGGAGGCTAGCTACAACGAGAGGAAACCT (SEQ ID NO: 190) |
| DZ-3 | CCAAAGGAGAGGCTAGCTACAACGAGGGACCCGT (SEQ ID NO: 191) |
| DZ-auto | AGCTGGGGAGGCTAGCTACAACGAGAGGGAGG (SEQ ID NO: 192) |
| Dz2- | GCCCAGGGAGGCTAGCTACAACGAGAGGAAACCTTTTTTAGGTTTCCT |
| hairpin- | CTCGTTGTAGCCTCCCTGGGC (SEQ ID NO: 193) |
| noRNA | |
| Dz2- | GCCCAGGGAGGCTAGCTACAACGAGAGGAAACCTTTTTTAGGTTTCCT |
| hairpin- | CTCGTTGrUrUTAGCCTCCCTGGGC (SEQ ID NO: 194) |
| U2 | |
| Dz2- | GCCCAGGGAGGCTAGCTACAACGAGAGGAAACCTTTTTTAGGTTTCCT |
| hairpin- | CTCGTTGrUrUrUrUrUTAGCCTCCCTGGGC (SEQ ID NO: 195) |
| U5 | |

In some embodiments, the catalytic oligonucleotide is inactive due to interference with and/or disruption of the secondary structure needed for its catalytic activity. Interference with and/or disruption of the secondary structure of catalytic oligonucleotide, such as to inhibit its activity can be accomplished in various ways, such as by circularization or binding to a blocker oligonucleotide.

In some embodiments, the catalytic oligonucleotide is circularized, which prevents the cleaving activity of the catalytic oligonucleotide. The circularized catalytic oligonucleotide can comprise a site that is cleaved by a programmable nuclease as described herein. Examples of this comprise ligating together the two ends of the catalytic oligonucleotide to form a circular structure of the catalytic oligonucleotide, rending it inactive. Upon binding to the target nucleic acid and subsequent activation of trans collateral cleavage, the programmable nuclease can cleave the circularized catalytic oligonucleotide. Upon cleavage of the circular catalytic oligonucleotide, the catalytic oligonucleotide can form a secondary structure that enables the catalytic oligonucleotide's catalytic activity, such as binding to a catalytic oligonucleotide recognition site in a reporter molecule or in a blocker oligonucleotide and cleaving that molecule.

In some embodiments, the catalytic oligonucleotide is bound to a blocker oligonucleotide, which prevents the cleaving activity of the catalytic oligonucleotide. The blocker oligonucleotide can bind or hybridize to a catalytic oligonucleotide, which alters the secondary structure of the catalytic oligonucleotide and therefore prevents the catalytic oligonucleotide from binding to its target and perform its cleavage activity. In some embodiments, the blocker oligonucleotide comprises a site that is cleaved by a programmable nuclease as described herein. In some embodiments, the blocker oligonucleotide comprises a site that is cleaved by a programmable nuclease and comprises a site that is cleaved by the catalytic oligonucleotide. Upon binding to the target nucleic acid and subsequent activation trans collateral cleavage, the programmable nuclease can cleave in the blocker oligonucleotide. Upon cleavage of the blocker oligonucleotide, the catalytic oligonucleotide can form a secondary structure that enables the catalytic oligonucleotide's catalytic activity, such as binding to and cleaving a reporter molecule and/or binding to and cleaving a blocker oligonucleotide.

In some embodiments, the first catalytic oligonucleotide is bound to a first blocker oligonucleotide and a second catalytic oligonucleotide is bound to a second blocker, which prevents the cleaving activity of the first catalytic oligonucleotide and prevents the cleavage activity of the second catalytic oligonucleotide. The first blocker oligonucleotide can bind or hybridize to a first catalytic oligonucleotide, which alters the secondary structure of the first catalytic oligonucleotide and therefore prevents the first catalytic oligonucleotide from binding to its target and perform its cleavage activity. The second blocker oligonucleotide can bind or hybridize to a second catalytic oligonucleotide, which alters the secondary structure of the second catalytic oligonucleotide and therefore prevents the second catalytic oligonucleotide from binding to its target and perform its cleavage activity. In some embodiments, the first blocker oligonucleotide comprises a site that is cleaved by a programmable nuclease and a second catalytic oligonucleotide binding site that is cleaved by the second catalytic oligonucleotide, and the second blocker oligonucleotide comprises a first catalytic oligonucleotide binding site that is cleaved by the first catalytic oligonucleotide. Upon binding to the target nucleic acid and subsequent activation trans collateral cleavage, the programmable nuclease can cleave in the first blocker oligonucleotide. Upon cleavage of the first blocker oligonucleotide, the first catalytic oligonucleotide can form a secondary structure that enables the first catalytic oligonucleotide's catalytic activity, such as binding to and cleaving a reporter molecule and/or binding to and cleaving the first catalytic oligonucleotide site in the second blocker oligonucleotide. The second catalytic oligonucleotide can cleave in the first blocker oligonucleotide at the second catalytic oligonucleotide binding site, allowing for the additional first catalytic oligonucleotides to cleave reporter molecules.

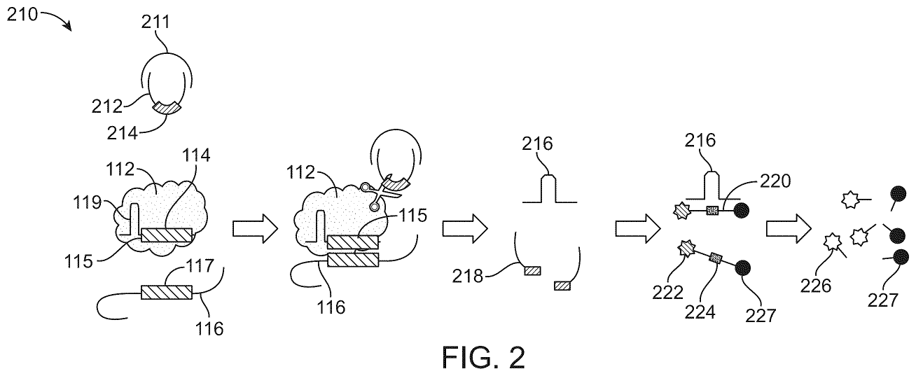

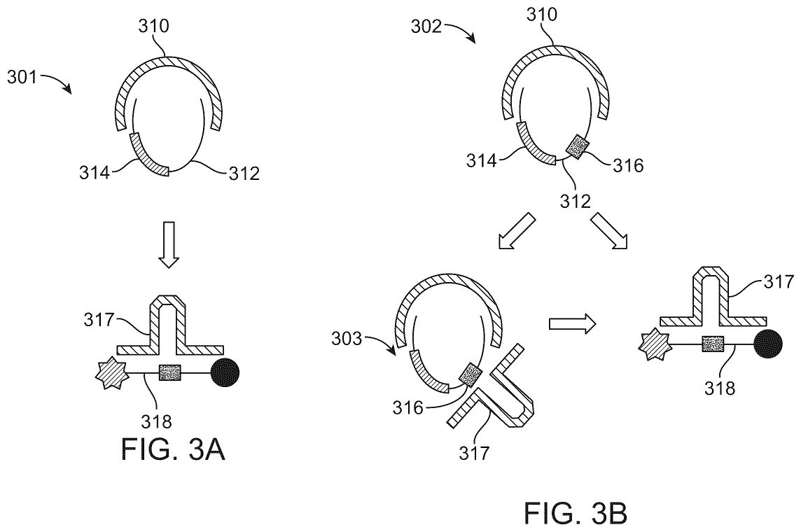

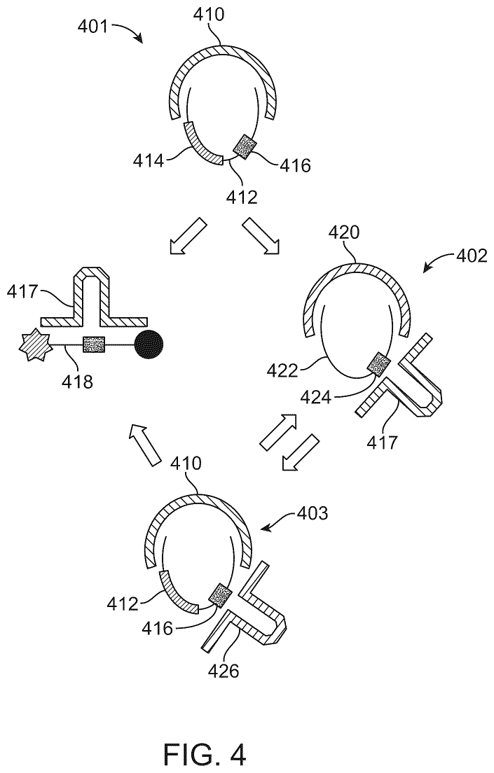

Blocker oligonucleotides and methods of use thereof are described in further detail herein, such as generally in FIG. 2, FIGS. 3A-3B, and FIG. 4. A list of exemplary sequences of blocker oligonucleotides which can be used in compositions and methods of the present disclosure are provided in TABLE 7.

TABLE 7
Exemplary Blocker Oligonucleotides

| | | Bound |
|---|---|---|
| Blocker | | Catalytic |
| Oligo- | | Oligo- |
| nucleotide | Sequence (SEQ ID NO:) | nucleotide |
| Dz2- | AGCCTCCCTGGGCATCGGGTCCCrUrUrUrUrUCTCCTTTGTAA | Dz2 |
| blocker- | GGTTTCCTCTCG (SEQ ID NO: 196) | |
| U5 | | |
| Dz3- | AGCCTCCCTGGGCATCGGGTCCCrGrUCTCCTTTGTAAGGTT | Dz-3 |
| blocker | TCCTCTCG (SEQ ID NO: 197) | |
| (mod001) | | |
| AutoBloc | AGCCTCCCCAGCTATCACrUrUrUrUrUAGCTCCTCCCTCrGrUC | DZ-auto |
| ker-1 | CCCAGCTTCCTCCCTCTCGTTG (SEQ ID NO: 198) | |
| AutoBloc | AGCCTCCCCAGCTATCACrUrUrUrUrUAGCTCGACCCCrGrUC | DZ-auto |
| ker-2 | TCCACGCCTCCTCCCTCTCGTTG (SEQ ID NO: 199) | |
| AutoBloc | AGCCTCCCCAGCTATCACrUrUrUrUrUAGCTCCTCCCTCTCGT | DZ-auto |
| ker-3 | TG (SEQ ID NO: 200) | |
| DBL- | TAGCCTTCCTCCCATCACrUrUrUrUrUAGCTGGTTTCCTCrGrU | Dz3 |
| blocker3 | CCCTGGGCACGCCTCTCGT (SEQ ID NO: 201) | |
| DBL- | CTAGCCTCCCTGGTCGGGTCCCrUrUrUrUrUCTCCTTTGTCAC | Dz2 |
| blocker-2 | GCCTCrGrUTCCTCCCAGTTTCCTCTCGTT (SEQ ID NO: 202) | |

### Reporters

Described herein are compositions and methods of use thereof comprising one or more reporter molecules. In some examples, the one or more reporter molecules comprise one or more different reporter molecules. In an example, the one or more reporter molecules comprise a first reporter molecule, a second reporter molecule, a third reporter molecule, and/or more reporter molecules or a plurality of each reporter molecule wherein each reporter molecule can be present in multiple copies (e.g., at a predefined concentration) in the composition. In some examples, the compositions and methods comprise 1, 2, 3, 4, 5, 6, 7, 8, 9, 10, or more reporter molecules or sequences.

By way of non-limiting and illustrative example, a reporter may comprise a single stranded nucleic acid and a detection moiety (e.g., a labeled single stranded RNA reporter), wherein the nucleic acid is capable of being cleaved by a programmable nuclease (e.g., a Type V CRISPR/Cas protein as disclosed herein) or a multimeric complex thereof, releasing the detection moiety, and, generating a detectable signal. As used herein, “reporter” is used interchangeably with “reporter nucleic acid” or “reporter molecule”. The programmable nucleases disclosed herein, activated upon hybridization of a guide RNA to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

Often, the reporter is a protein-nucleic acid. For example, a method of assaying for a target nucleic acid in a sample comprises contacting the sample to a complex comprising a) a guide nucleic acid comprising a segment that is reverse complementary to a segment of the target nucleic acid and b) a programmable nuclease that exhibits sequence independent cleavage upon forming an activated complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. Often, the protein-nucleic acid is an enzyme-nucleic acid or an enzyme substrate-nucleic acid. Sometimes, the protein-nucleic acid is attached to a solid support. The nucleic acid can be DNA, RNA, or a DNA/RNA hybrid. The methods described herein use a programmable nuclease, such as the CRISPR/Cas system, to detect a target nucleic acid. A method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Cleavage of the protein-nucleic acid produces a signal. The systems and devices disclosed herein can be used to detect these signals, which indicate whether a target nucleic acid is present in the sample.

In some examples, a reporter molecule is a single stranded reporter molecule comprising a detection moiety, wherein the reporter molecule is capable of being cleaved by the activated programmable nuclease, thereby generating a first detectable signal. In some cases, the reporter molecule is a single-stranded nucleic acid sequence comprising ribonucleotides. In some cases, the reporter molecule is a single-stranded nucleic acid sequence comprising deoxyribonucleotides. In some cases, the reporter molecule is a single-stranded nucleic acid sequence comprising deoxyribonucleotides and ribonucleotides. As described herein, nucleic acid sequences can be detected using a programmable RNA nuclease, a programmable DNA nuclease, or a combination thereof, as disclosed herein. The programmable nuclease can be activated and cleave the reporter molecule upon binding of a guide nucleic acid to a target nucleic acid. Additionally, different compositions of reporter molecules can allow for multiplexing using different programmable nucleases (e.g., a programmable RNA nuclease and a programmable DNA nuclease).

The reporter molecule can be a single-stranded nucleic acid sequence comprising at least one deoxyribonucleotide and at least one ribonucleotide. In some cases, the reporter molecule is a single-stranded nucleic acid comprising at least one ribonucleotide residue at an internal position that functions as a cleavage site. In some cases, the reporter molecule comprises at least 2, 3, 4, 5, 6, 7, 8, 9, or 10 ribonucleotide residues at an internal position. In some cases, the reporter molecule comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the reporter molecule has only ribonucleotide residues. In some cases, the reporter molecule has only deoxyribonucleotide residues. In some cases, the reporter molecule comprises nucleotides resistant to cleavage by the programmable nuclease described herein. In some cases, the reporter molecule comprises synthetic nucleotides. In some cases, the reporter molecule comprises at least one ribonucleotide residue and at least one non-ribonucleotide residue. In some cases, the reporter molecule is 5-20, 5-15, 5-10, 7-20, 7-15, or 7-10 nucleotides in length. In some cases, the reporter molecule is from 3 to 20, from 4 to 10, from 5 to 10, or from 5 to 8 nucleotides in length. In some cases, the reporter molecule comprises at least one uracil ribonucleotide. In some cases, the reporter molecule comprises at least two uracil ribonucleotides. Sometimes the reporter molecule has only uracil ribonucleotides. In some cases, the reporter molecule comprises at least one adenine ribonucleotide. In some cases, the reporter molecule comprises at least two adenine ribonucleotide. In some cases, the reporter molecule has only adenine ribonucleotides. In some cases, the reporter molecule comprises at least one cytosine ribonucleotide. In some cases, the reporter molecule comprises at least two cytosine ribonucleotide. In some cases, the reporter molecule comprises at least one guanine ribonucleotide. In some cases, the reporter molecule comprises at least two guanine ribonucleotide. A reporter molecule can comprise only unmodified ribonucleotides, only unmodified deoxyribonucleotides, or a combination thereof. In some cases, the reporter molecule is from 5 to 12 nucleotides in length. In some cases, the reporter molecule is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the reporter molecule is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a reporter molecule can be 5, 8, or 10 nucleotides in length. For cleavage by a programmable nuclease comprising Cas12, a reporter molecule can be 10 nucleotides in length.

In some embodiments, the single stranded reporter molecule comprises a detection moiety capable of generating a first detectable signal. Sometimes the reporter molecule comprises a protein capable of generating a signal. A signal can be a calorimetric, potentiometric, amperometric, optical (e.g., fluorescent, colorometric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the reporter molecule. Sometimes the detection moiety is at the 3′ terminus of the reporter molecule. In some cases, the detection moiety is at the 5′ terminus of the reporter molecule. In some cases, the quenching moiety is at the 3′ terminus of the reporter molecule. In some cases, the single-stranded reporter molecule is at least one population of the single-stranded nucleic acid capable of generating a first detectable signal. In some cases, the single-stranded reporter molecule is a population of the single stranded nucleic acid capable of generating a first detectable signal. Optionally, there are more than one population of single-stranded reporter molecule. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded reporter molecules capable of generating a detectable signal. In some cases there are from 2 to 50, from 3 to 40, from 4 to 30, from 5 to 20, or from 6 to 10 different populations of single-stranded reporter molecules capable of generating a detectable signal. TABLE 8 provides a list of exemplary fluorescent reporter molecules that are bound and activated by DNAzymes. TABLE 9 provides a list of exemplary single stranded reporter molecules. In some embodiments, different fluorescent reporter molecules (e.g., different color fluorescent reporter molecules), are used as a means of differentiating between the programmable nuclease trans-collateral cleaving activity and the catalytic oligonucleotide cleaving activity.

TABLE 8
Exemplary Fluorescent Reporter Molecules

| 5′ | | | |
|---|---|---|---|
| Detection | | | Specific |
| Moiety* | Sequence (SEQ ID NO:) | 3′ Quencher* | For |
| /56-FAM/ | CCAGCCTTTTTCTGCTAGrArUTACCTGTA | /3IABKFQ/ | Circularized |
| | TTTTGGCTGG (SEQ ID NO: 203) | | Dz1 and Dz- |
| | | | act-linear |
| /56-FAM/ | AAGGTTTCCTCrGrUCCCTGGGCA (SEQ | /3IABkFQ | Dz2, |
| | ID NO: 204) | | hairpin |
| | | | Dz′s |
| /56-FAM/ | CTCCTCCCTCrGrUCCCCAGCTC (SEQ ID | /3IABKFQ/ | Dz-auto |
| | NO: 205) | | |

/56-FAM/: 5′ 6-Fluorescein (Integrated DNA Technologies)
*This Table refers to the detection moiety and quencher moiety as their tradenames and their source is identified. However, alternatives, generics, or non-tradename moieties with similar function from other sources can also be used.

TABLE 9
Exemplary Single Stranded Reporter Molecules

| 5′ Detection | | |
|---|---|---|
| Moiety* | Sequence (SEQ ID NO:) | 3′ Quencher* |
| /56-FAM/ | rUrUrUrUrU (SEQ ID NO: 1) | /3IABKFQ/ |
| /5IRD700/ | rUrUrUrUrU (SEQ ID NO: 1) | /3IRQCIN/ |
| /5TYE665/ | rUrUrUrUrU (SEQ ID NO: 1) | /3IAbRQSp/ |
| /5Alex594N/ | rUrUrUrUrU (SEQ ID NO: 1) | /3IAbRQSp/ |
| /5ATTO633N/ | rUrUrUrUrU (SEQ ID NO: 1) | /3IAbRQSp/ |
| /56-FAM/ | rUrUrUrUrUrUrUrU(SEQ ID NO: 2) | /3IABKFQ/ |
| /5IRD700/ | rUrUrUrUrUrUrUrU(SEQ ID NO: 2) | /3IRQCIN/ |
| /5TYE665/ | rUrUrUrUrUrUrUrU(SEQ ID NO: 2) | /3IAbRQSp/ |
| /5Alex594N/ | rUrUrUrUrUrUrUrU(SEQ ID NO: 2) | /3IAbRQSp/ |
| /5ATTO633N/ | rUrUrUrUrUrUrUrU(SEQ ID NO: 2) | /3IAbRQSp/ |
| /56-FAM/ | rUrUrUrUrUrUrUrUrUrU(SEQ ID NO: 3) | /3IABKFQ/ |
| /5IRD700/ | rUrUrUrUrUrUrUrUrUrU(SEQ ID NO: 3) | /3IRQCIN/ |
| /5TYE665/ | rUrUrUrUrUrUrUrUrUrU(SEQ ID NO: 3) | /3IAbRQSp/ |
| /5Alex594N/ | rUrUrUrUrUrUrUrUrUrU(SEQ ID NO: 3) | /3IAbRQSp/ |
| /5ATTO633N/ | rUrUrUrUrUrUrUrUrUrU(SEQ ID NO: 3) | /3IAbRQSp/ |
| /56-FAM/ | TTTTrUrUTTTT(SEQ ID NO: 4) | /3IABKFQ/ |
| /5IRD700/ | TTTTrUrUTTTT(SEQ ID NO: 4) | /3IRQCIN/ |
| /5TYE665/ | TTTTrUrUTTTT(SEQ ID NO: 4) | /3IAbRQSp/ |
| /5Alex594N/ | TTTTrUrUTTTT(SEQ ID NO: 4) | /3IAbRQSp/ |
| /5ATTO633N/ | TTTTrUrUTTTT(SEQ ID NO: 4) | /3IAbRQSp/ |
| /56-FAM/ | TTrUrUTT(SEQ ID NO: 5) | /3IABKFQ/ |
| /5IRD700/ | TTrUrUTT(SEQ ID NO: 5) | /3IRQCIN/ |
| /5TYE665/ | TTrUrUTT(SEQ ID NO: 5) | /3IAbRQSp/ |
| /5Alex594N/ | TTrUrUTT(SEQ ID NO: 5) | /3IAbRQSp/ |
| /5ATTO633N/ | TTrUrUTT(SEQ ID NO: 5) | /3IAbRQSp/ |
| /56-FAM/ | TArArUGC(SEQ ID NO: 6) | /3IABKFQ/ |
| /5IRD700/ | TArArUGC(SEQ ID NO: 6) | /3IRQCIN/ |
| /5TYE665/ | TArArUGC(SEQ ID NO: 6) | /3IAbRQSp/ |
| /5Alex594N/ | TArArUGC(SEQ ID NO: 6) | /3IAbRQSp/ |
| /5ATTO633N/ | TArArUGC(SEQ ID NO: 6) | /3IAbRQSp/ |
| /56-FAM/ | TArUrGGC(SEQ ID NO: 7) | /3IABKFQ/ |
| /5IRD700/ | TArUrGGC(SEQ ID NO: 7) | /3IRQCIN/ |
| /5TYE665/ | TArUrGGC(SEQ ID NO: 7) | /3IAbRQSp/ |
| /5Alex594N/ | TArUrGGC(SEQ ID NO: 7) | /3IAbRQSp/ |
| /5ATTO633N/ | TArUrGGC(SEQ ID NO: 7) | /3IAbRQSp/ |
| /56-FAM/ | rUrUrUrUrU(SEQ ID NO: 8) (Rep. ID: | /3IABKFQ/ |
| | rep01) | |
| /5IRD700/ | rUrUrUrUrU(SEQ ID NO: 8) | /3IRQCIN/ |
| /5TYE665/ | rUrUrUrUrU(SEQ ID NO: 8) | /3IAbRQSp/ |
| /5Alex594N/ | rUrUrUrUrU(SEQ ID NO: 8) | /3IAbRQSp/ |
| /5ATTO633N/ | rUrUrUrUrU(SEQ ID NO: 8) | /3IAbRQSp/ |
| /56-FAM/ | TTATTATT (SEQ ID NO: 9) | /3IABKFQ/ |
| /56-FAM/ | TTATTATT (SEQ ID NO: 9) | /3IABKFQ/ |
| /5IRD700/ | TTATTATT (SEQ ID NO: 9) | /3IRQCIN/ |
| /5TYE665/ | TTATTATT (SEQ ID NO: 9) | /3IAbRQSp/ |
| /5Alex594N/ | TTATTATT (SEQ ID NO: 9) | /3IAbRQSp/ |
| /5ATTO633N/ | TTATTATT (SEQ ID NO: 9) | /3IAbRQSp/ |
| /56-FAM/ | TTTTTT (SEQ ID NO: 10) | /3IABKFQ/ |
| /56-FAM/ | TTTTTTTT (SEQ ID NO: 11) | /3IABKFQ/ |
| /56-FAM/ | TTTTTTTTTT (SEQ ID NO: 12) | /3IABKFQ/ |
| /56-FAM/ | TTTTTTTTTTTT (SEQ ID NO: 13) | /3IABKFQ/ |
| /56-FAM/ | TTTTTTTTTTTTTT (SEQ ID NO: 14) | /3IABKFQ/ |
| /56-FAM/ | AAAAAA (SEQ ID NO: 15) | /3IABKFQ/ |
| /56-FAM/ | CCCCCC (SEQ ID NO: 16) | /3IABKFQ/ |
| /56-FAM/ | GGGGGG (SEQ ID NO: 17) | /3IABKFQ/ |
| /56-FAM/ | TTATTATT (SEQ ID NO: 9) | /3IABKFQ/ |

/56-FAM/: 5′ 6-Fluorescein (Integrated DNA Technologies)
/3IABKFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester) (Integrated DNA Technologies)
/5ATTO633N/: 5′ ATTO TM 633 (NHS Ester) (Integrated DNA Technologies)
/3IRQCIN/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

A detection moiety can be a fluorophore. The detection moiety can be a fluorophore that emits fluorescence in the visible spectrum. In some embodiments, the detection moiety can be a fluorophore that emits fluorescence in the visible spectrum. In some embodiments, the detection moiety can be a fluorophore that emits fluorescence in the near-IR spectrum. In some embodiments, the detection moiety can be a fluorophore that emits fluorescence in the IR spectrum. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the detection moiety emits fluorescence in the range from 450 nm to 750 nm, from 500 nm to 650 nm, or from 550 to 650 nm. A detection moiety can be a fluorophore that emits a fluorescence in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, (E≤−glucuronidase, invertase, Xanthine Oxidase, firefly luciferase, and glucose oxidase (GO).

A detection moiety can be chosen for use based on the type of sample to be tested. For example, a detection moiety that is an infrared fluorophore is used with a urine sample. As another example, SEQ ID NO: 1 with a fluorophore that emits a fluorescence around 520 nm is used for testing in non-urine samples, and SEQ ID NO: 8 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluoresecence in the range from 450 nm to 750 nm, from 500 nm to 650 nm, or from 550 to 650 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies), Black Hole Quencher (Sigma Aldrich), or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nuclease has occurred and that the sample contains the target nucleic acid. In addition, in some examples, a catalytic oligonucleotide can be activated by the programmable nuclease upon its hybridization to the target nucleic acid molecule. In some instances, a catalytic oligonucleotide can be used to further intensify the detectable signal. This can decrease the detection threshold. For examples, analytes (e.g., target nucleic acid molecules) at lower concentrations can be detected using the assay as the assay sensitivity can be increased using a catalytic oligonucleotide as described herein.

In some cases, the detection moiety comprises a fluorescent dye. In some examples, the detection moiety comprises a fluorescence resonance energy transfer (FRET) pair. In some cases, the detection moiety comprises an infrared (IR) dye. In some cases, the detection moiety comprises an ultraviolet (UV) dye. Alternatively, or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorometric, etc.), or piezo-electric signal. A reporter molecule, sometimes, is protein-nucleic acid that is capable of generating a calorimetric, potentiometric, amperometric, optical (e.g., fluorescent, colorometric, etc.), or piezo-electric signal upon cleavage of the nucleic acid. Often a calorimetric signal is heat produced after cleavage of the reporter molecules. Sometimes, a calorimetric signal is heat absorbed after cleavage of the reporter molecules. A potentiometric signal, for example, is electrical potential produced after cleavage of the reporter molecules. An amperometric signal can be movement of electrons produced after the cleavage of reporter molecule. Often, the signal is an optical signal, such as a colorometric signal or a fluorescence signal. An optical signal is, for example, a light output produced after the cleavage of the reporter molecules. Sometimes, an optical signal is a change in light absorbance between before and after the cleavage of reporter molecules. Often, a piezo-electric signal is a change in mass between before and after the cleavage of the reporter molecule. Other methods of detection can also be used, such as optical imaging, surface plasmon resonance (SPR), and/or interferometric sensing.

Often, the protein-nucleic acid is an enzyme-nucleic acid. The enzyme can be sterically hindered when present as in the enzyme-nucleic acid, but then functional upon cleavage from the nucleic acid. Often, the enzyme is an enzyme that produces a reaction with a substrate. An enzyme can be invertase. Often, the substrate of invertase is sucrose and DNS reagent. In some cases, it is preferred that the nucleic acid and invertase are conjugated using a heterobifunctional linker via sulfo-SMCC chemistry.

In some examples, the protein-nucleic acid is a substrate-nucleic acid. Often the substrate is a substrate that produces a reaction with an enzyme. Release of the substrate upon cleavage by the programmable nuclease may free the substrate to react with the enzyme.

A protein-nucleic acid or other reporter molecule can be attached to a solid support. The solid support, for example, is a surface. A surface can be an electrode. Sometimes the solid support is a bead. Often the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

Detecting the presence or absence of a target nucleic acid of interest can involve measuring a signal emitted from a detection moiety present in a reporter, after cleavage of the reporter by an activated programmable nuclease. The signal can be measured using one or more sensors integrated with the device or operatively coupled to a device. Thus, the detecting steps disclosed herein can involve measuring the presence of a target nucleic acid, quantifying how much of the target nucleic acid is present, or, measuring a signal indicating that the target nucleic acid is absent in a sample. In some embodiments, a signal is generated upon cleavage of the nucleic acid of the reporter by the programmable nuclease and/or a signal amplifier. In other embodiments, the signal changes upon cleavage of the reporter by the programmable nuclease and/or the signal amplifier. In other embodiments, a signal can be present in the absence of reporter cleavage and disappear upon cleavage of the target nucleic acid by the programmable nuclease and/or the signal amplifier. For example, a signal can be produced in a microfluidic device or lateral flow device after contacting a sample with a composition comprising a programmable nuclease and a signal amplifier as described herein.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorometric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system is capable of detecting more than one type of target nucleic acid, wherein the system comprises more than one type of guide nucleic acid and more than one type of reporter molecule. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the one or more detectable signals generated after cleavage can produce an index of refraction change or one or more electrochemical changes. In some cases, real-time detection of the Cas reaction can be achieved using fluorescence, electrochemical detection, and/or electrochemiluminescence.

In some cases, the detectable signals can be detected and analyzed in various ways. For example, the detectable signals can be detected using an imaging device. The imaging device can a digital camera, such a digital camera on a mobile device. The mobile device can have a software program or a mobile application that can capture fluorescence, ultraviolet (UV), infrared (IR), or visible wavelength signals. Any suitable detection or measurement device can be used to detect and/or analyze the colorimetric, fluorescence, amperometric, potentiometric, or electrochemical signals described herein. In some embodiments, the colorimetric, fluorescence, amperometric, potentiometric, or another electrochemical sign can be detected using a measurement device connected to a detection chamber of the device (e.g., a fluorescence measurement device, a spectrophotometer, and/or an oscilloscope).

In some embodiments, the reporter may comprise a nucleic acid and a detection moiety. In some embodiments, a reporter is connected to a surface by a linkage. In some embodiments, a reporter may comprise at least one of a nucleic acid, a chemical functionality, a detection moiety, a quenching moiety, or a combination thereof. In some embodiments, a reporter is configured for the detection moiety to remain immobilized to the surface and the quenching moiety to be released into solution upon cleavage of the reporter. In some embodiments, a reporter is configured for the quenching moiety to remain immobilized to the surface and for the detection moiety to be released into solution, upon cleavage of the reporter. Often the detection moiety is at least one of a label, a polypeptide, a dendrimer, or a nucleic acid, or a combination thereof. In some embodiments, the reporter contains a label. In some embodiments, label may be FITC, DIG, TAMRA, Cy5, AF594, or Cy3. In some embodiments, the label may comprise a dye, a nanoparticle configured to produce a signal. In some embodiments, the dye may be a fluorescent dye. In some embodiments, the at least one chemical functionality may comprise biotin. In some embodiments, the at least one chemical functionality may be configured to be captured by a capture probe. In some embodiments, the at least one chemical functionality may comprise biotin and the capture probe may comprise anti-biotin, streptavidin, avidin or other molecule configured to bind with biotin. In some embodiments, the dye is the chemical functionality. In some embodiments, a capture probe may comprise a molecule that is complementary to the chemical functionality. In some embodiments, the capture antibodies are anti-FITC, anti-DIG, anti-TAMRA, anti-Cy5, anti-AF594, or any other appropriate capture antibody capable of binding the detection moiety or conjugate. In some embodiments, the detection moiety can be the chemical functionality.

In some instances, reporters comprise a detection moiety capable of generating a signal. A signal may be a calorimetric, potentiometric, amperometric, optical (e.g., fluorescent, colorimetric, etc.), or piezo-electric signal. In some cases, the reporter comprises a detection moiety. Suitable detectable labels and/or moieties that may provide a signal include, but are not limited to, an enzyme, a radioisotope, a member of a specific binding pair, a fluorophore, a fluorescent protein, a quantum dot, and the like.

In some embodiments, the reporter comprises a nucleic acid conjugated to an affinity molecule which is in turn conjugated to the fluorophore (e.g., nucleic acid—affinity molecule—fluorophore) or the nucleic acid conjugated to the fluorophore which is in turn conjugated to the affinity molecule (e.g., nucleic acid—fluorophore—affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the reporter can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicates that no intervening molecules, polypeptides, proteins, or other moieties are present between the two moieties directly conjugated to each other. For example, if a reporter comprises a nucleic acid directly conjugated to an affinity molecule and an affinity molecule directly conjugated to a fluorophore—no intervening moiety is present between the nucleic acid and the affinity molecule and no intervening moiety is present between the affinity molecule and the fluorophore. The affinity molecule can be biotin, avidin, streptavidin, or any similar molecule.

In some cases, the reporter comprises a substrate-nucleic acid. The substrate may be sequestered from its cognate enzyme when present as in the substrate-nucleic acid, but then is released from the nucleic acid upon cleavage, wherein the released substrate can contact the cognate enzyme to produce a detectable signal. Often, the substrate is sucrose and the cognate enzyme is invertase, and a DNS reagent can be used to monitor invertase activity.

A reporter may be a hybrid nucleic acid reporter. A hybrid nucleic acid reporter comprises a nucleic acid with at least one deoxyribonucleotide and at least one ribonucleotide. In some embodiments, the nucleic acid of the hybrid nucleic acid reporter can be of any length and can have any mixture of DNAs and RNAs. For example, in some cases, longer stretches of DNA can be interrupted by a few ribonucleotides. Alternatively, longer stretches of RNA can be interrupted by a few deoxyribonucleotides. Alternatively, every other base in the nucleic acid may alternate between ribonucleotides and deoxyribonucleotides. A major advantage of the hybrid nucleic acid reporter is increased stability as compared to a pure RNA nucleic acid reporter. For example, a hybrid nucleic acid reporter can be more stable in solution, lyophilized, or vitrified as compared to a pure DNA or pure RNA reporter.

The reporter can be lyophilized or vitrified. The reporter can be suspended in solution or immobilized on a surface. For example, the reporter can be immobilized on the surface of a chamber in a device as disclosed herein. In some cases, the reporter is immobilized on beads, such as magnetic beads, in a chamber of a device as disclosed herein where they can be held in position by a magnet placed below the chamber.

Additionally, target nucleic acid can optionally be amplified before binding to the guide nucleic acid (e.g., crRNA) of the programmable nuclease (e.g., CRISPR enzyme). This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. Sometimes, the nucleic acid amplification reaction is performed at a temperature of around 45-65° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C.

Disclosed herein are methods of assaying for a target nucleic acid as described herein wherein a signal is detected. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a reaction substrate; c) contacting the reaction substrate to a reagent that differentially reacts with a cleaved substrate; and d) assaying for a signal indicating cleavage of the reaction substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby nucleic acids by the activated programmable nuclease, such as trans cleavage of reporters with a detection moiety. Once the reporter is cleaved by the activated programmable nuclease, the detection moiety can be released from the reporter and can generate a signal. The signal can be detected from a detection spot on a support medium, wherein the detection spot comprises capture probes for cleaved reporter fragments. The signal can be visualized to assess whether a target nucleic acid comprises a modification.

Often, the signal is a colorimetric signal or a signal visible by eye. In some cases, the first detection signal is generated by binding of the detection moiety to a capture molecule in a detection region, where the first detection signal indicates that the sample contained the target nucleic acid. Sometimes the system is capable of detecting more than one type of target nucleic acid, wherein the system comprises more than one type of guide nucleic acid and more than one type of reporter capable of directly or indirectly generating at least a first detection signal and a second detection signal. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on the spatial location of the detectable signal on the detection region of the support medium. In some cases, the second detectable signal is generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, fom 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the systems, devices, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some instances, systems comprise a Type V CRISPR/Cas protein and a reporter nucleic acid configured to undergo transcollateral cleavage by the Type V CRISPR/Cas protein. Transcollateral cleavage of the reporter may generate a signal from the reporter or alter a signal from the reporter. In some cases, the signal is an optical signal, such as a fluorescence signal or absorbance band. Transcollateral cleavage of the reporter may alter the wavelength, intensity, or polarization of the optical signal. For example, the reporter may comprise a fluorophore and a quencher, such that transcollateral cleavage of the reporter separates the fluorophore and the quencher thereby increasing a fluorescence signal from the fluorophore. Herein, detection of reporter cleavage to determine the presence of a target nucleic acid sequence may be referred to as ‘DETECTR’. In some embodiments described herein is a method of assaying for a target nucleic acid in a sample comprising contacting the target nucleic acid with a programmable nuclease, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of a programmable nuclease (e.g., a Type V CRISPR/Cas protein as disclosed herein) may be inhibited. If total nucleic acids are present in large amounts, they may outcompete reporters for the programmable nucleases. In some instances, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some instances, the sample comprises amplified target nucleic acid. In some instances, the sample comprises an unamplified target nucleic acid. In some instances, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some instances, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### DETECTR Immobilization

One or more components or reagents of a programmable nuclease-based detection reaction may be suspended in solution or immobilized on a surface. Programmable nucleases, guide nucleic acids, and/or reporters may be suspended in solution or immobilized on a surface. For example, the reporter, programmable nuclease, and/or guide nucleic acid can be immobilized on the surface of a chamber in a device as disclosed herein. In some cases, the reporter, programmable nuclease, and/or guide nucleic acid can be immobilized on beads, such as magnetic beads, in a chamber of a device as disclosed herein where they are held in position by a magnet placed below the chamber. An immobilized programmable nuclease can be capable of being activated and cleaving a free-floating or immobilized reporter. An immobilized guide nucleic acid can be capable of binding a target nucleic acid and activating a programmable nuclease complexed thereto. An immobilized reporter can be capable of being cleaved by the activated programmable nuclease, thereby generating a detectable signal.

Described herein are various methods to immobilize programmable nuclease-based diagnostic reaction components to the surface of a reaction chamber or other surface (e.g., a surface of a bead). Any of the devices described herein may comprise one or more immobilized detection reagent components (e.g., programmable nuclease, guide nucleic acid, and/or reporter). In certain instances, methods include immobilization of programmable nucleases (e.g., Cas proteins or Cas enzymes), reporters, and guide nucleic acids (e.g., gRNAs). In some embodiments, various programmable nuclease-based diagnostic reaction components are modified with biotin. In some embodiments, these biotinylated programmable nuclease-based diagnostic reaction components are immobilized on surfaces coated with streptavidin. In some embodiments, the biotin-streptavidin chemistries are used for immobilization of programmable nuclease-based reaction components. In some embodiments, NHS-Amine chemistry is used for immobilization of programmable nuclease-based reaction components. In some embodiments, amino modifications are used for immobilization of programmable nuclease-based reaction components.

In some embodiments, the programmable nuclease, guide nucleic acid, or the reporter are immobilized to a device surface by a linkage or linker. In some embodiments, the linkage comprises a covalent bond, a non-covalent bond, an electrostatic bond, a bond between streptavidin and biotin, an amide bond or any combination thereof. In some embodiments, the linkage comprises non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface.

In some embodiments, the programmable nuclease, guide nucleic acid, or the reporter are immobilized to or within a polymer matrix. The polymer matrix may comprise a hydrogel. Co-polymerization of the programmable nuclease, guide nucleic acid, or the reporter into the polymer matrix may result in a higher density of reporter/unit volume or reporter/unit area than other immobilization methods utilizing surface immobilization (e.g., onto beads, after matrix polymerization, etc.). Co-polymerization of the programmable nuclease, guide nucleic acid, or the reporter into the polymer matrix may result in less undesired release of the reporter (e.g., during an assay, a measurement, or on the shelf), and thus may cause less background signal, than other immobilization strategies (e.g., conjugation to a pre-formed hydrogel, bead, etc.). In at least some instances this may be due to better incorporation of reporters into the polymer matrix as a co-polymer and fewer “free” reporter molecules retained on the hydrogel via non-covalent interactions or non-specific binding interactions.

In some embodiments, a plurality of oligomers and a plurality of polymerizable oligomers may comprise an irregular or non-uniform mixture. The irregularity of the mixture of polymerizable oligomers and unfunctionalized oligomers may allow pores to form within the hydrogel (i.e., the unfunctionalized oligomers may act as a porogen). For example, the irregular mixture of oligomers may result in phase separation during polymerization that allows for the generation of pores of sufficient size for free-floating programmable nucleases to diffuse into the hydrogel and access immobilized internal reporter molecules. The relative percentages and/or molecular weights of the oligomers may be varied to vary the pore size of the hydrogel. For example, pore size may be tailored to increase the diffusion coefficient of the programmable nucleases.

In some embodiments, the functional groups attached to the reporters and/or guide nucleic acids may be selected to preferentially incorporate the reporters and/or guide nucleic acids into the polymer matrix via covalent binding at the functional group versus other locations along the nucleic acid backbone of the reporter and/or guide nucleic acid. In some embodiments, the functional groups attached to the reporters and/or guide nucleic acids may be selected to favorably transfer free radicals from the functionalized ends of polymerizable oligomers to the functional group on the end of the reporter and/or guide nucleic acid (e.g., 5′ end), thereby forming a covalent bond and immobilizing the reporter and/or guide nucleic acid rather than destroying other parts of the reporter and/or guide nucleic acid molecules, respectively. In some embodiments, the functional group may comprise a single stranded nucleic acid, a double stranded nucleic acid, an acrydite group, a 5′ thiol modifier, a 3′ thiol modifier, an amine group, a I-Linker™ group, methacryl group, or any combination thereof. One of ordinary skill in the art will recognize that a variety of functional groups may be used depending on the desired properties of the immobilized components.

### Modified Nucleic Acids

In some cases, a reporter and/or guide nucleic acid can comprise one or more modifications, e.g., a vase modification, a backbone modification, a sugar modification, etc., to provide the nucleic acid with a new or enhanced feature (e.g., improved stability).

Examples of suitable modifications include modified nucleic acid backbones and non-natural intemucleoside linkages. Nucleic acids having modified backbones can include those that retain a phosphorus atom in the backbone and those that do not have a phosphorus atom in the backbone. Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included. Also suitable are nucleic acids having morpholino backbone structures. Suitable modified polynucleotide backbones that do not include a phosphorus atom therein have backbones that are formed by short chain alkyl or cycloalkyl internucleoside linkages, mixed heteroatom and alkyl or cycloalkyl intemucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Other suitable modifications include nucleic acid mimetics. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone. Another such mimetic is a morpholino-based polynucleotide based on linked morpholino units (morpholino nucleic acid) having heterocyclic bases attached to the morpholino ring. A further class of nucleic acid mimetic is referred to as a cyclohexenyl nucleic acid (CeNA). The furanose ring normally present in a DNA/RNA molecule is replaced with a cyclohexenyl ring. Another modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety.

The nucleic acids described herein can include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C10 alkyl or C2 to C10 alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

Other suitable sugar substituent groups include methoxy (—O—CH₃), aminopropoxy (—OCH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

The nucleic acids described herein may include nucleobase modifications or substitutions. A labeled detector ssDNA (and/or a guide RNA) may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,2′:4,5)pyrrolo(2,3-d)pyrimidin-2-one). Heterocyclic base moieties may also include those in which the purine or pyrimidine base is replaced with other heterocycles, for example 7-deaza-adenine, 7-deazaguanosine, 2-aminopyridine, and 2-pyridone.

The nucleic acids described and referred to herein can comprise a plurality of base pairs. A base pair can be a biological unit comprising two nucleobases bound to each other by hydrogen bonds. Nucleobases can comprise adenine, guanine, cytosine, thymine, and/or uracil. In some cases, the nucleic acids described and referred to herein can comprise different base pairs. In some cases, the nucleic acids described and referred to herein can comprise one or more modified base pairs. The one or more modified base pairs can be produced when one or more base pairs undergo a chemical modification leading to new bases. The one or more modified base pairs can be, for example, Hypoxanthine, Inosine, Xanthine, Xanthosine, 7-Methylguanine, 7-Methylguanosine, 5,6-Dihydrouracil, Dihydrouridine, 5-Methylcytosine, 5-Methylcytidine, 5-hydroxymethylcytosine (5hmC), 5-formylcytosine (5fC), or 5-carboxylcytosine (5caC).

### Target Nucleic Acid

Disclosed herein are compositions, systems and methods for detecting a target nucleic acid. In some instances, the target nucleic acid is a single stranded nucleic acid. Alternatively, or in combination, the target nucleic acid is a double stranded nucleic acid and is prepared into single stranded nucleic acids before or upon contacting the programmable nuclease-based detection reagents (e.g., programmable nuclease, guide nucleic acid, and/or reporter). In some embodiments, the target nucleic acid is a double stranded nucleic acid. A target nucleic acid as described herein can be a target DNA. A target nucleic acid as described herein can be a target RNA. In some embodiments, the target RNA is reverse transcribed into a target DNA, and the target DNA binds to the programmable nuclease for activation of trans collateral cleavage. In some embodiments, the target DNA is transcribed into a target RNA, and the target RNA binds to the programmable nuclease for activation of trans collateral cleavage. The target nucleic acids include but are not limited to mRNA, rRNA, tRNA, non-coding RNA, long non-coding RNA, and microRNA (miRNA). In some instances, the target nucleic acid is complementary DNA (cDNA) synthesized from a single-stranded RNA template in a reaction catalyzed by a reverse transcriptase. In some cases, the target nucleic acid is single-stranded RNA (ssRNA) or mRNA. In some cases, the target nucleic acid is from a virus, a parasite, or a bacterium described herein.

A target nucleic acid as described herein can be in a sample. A variety of samples can be processed and/or analyzed using the methods, reagents, enzymes, and kits disclosed herein. In some embodiments, described herein are samples that contain deoxyribonucleic acid (DNA), which can be directly detected by a programmable DNA nuclease, such as a type V CRISPR enzyme. Type V CRISPR/Cas enzymes can be a Cas12 protein, a Cas14 protein, or a Case protein. A Cas12 protein can be a Cas12a (also referred to as Cpfl) protein, a Cas12b protein, Cas12c protein, Cas12d protein, or a Cas12e protein. A Cas14 protein can be a Cas14a protein, a Cas14b protein, a Cas14c protein, a Cas14d protein, a Cas14e protein, a Cas 14f protein, a Cas14g protein, a Cas14h protein, a Cas14i protein, a Cas14j protein, or a Cas14k protein. In some embodiments, described herein are samples that contain ribonucleic acid (RNA), which can be directly detected by a programmable RNA nuclease, such as a type VI CRISPR enzyme, for example Cas13a, Cas13b, Cas13c, Cas13d, or Cas13e. In some embodiments, described herein are samples that contain deoxyribonucleic acid (DNA), which can be directly detected by a programmable RNA nuclease, such as a type VI CRISPR enzyme, for example Cas13a, Cas13b, Cas13c, Cas13d, or Cas13e. As described herein, a target nucleic acid can be directly detected using a programmable nuclease as disclosed herein. Direct target nucleic acid detection using a programmable nuclease can eliminate the need for intermediate steps, such as reverse transcription or amplification. Elimination of said intermediate steps decreases time to assay result and reduces labor and reagent costs.

A programmable nuclease-guide nucleic acid complex may comprise high selectivity for a target sequence. In some cases, a ribonucleoprotein may comprise a selectivity of at least 200:1, 100:1, 50:1, 20:1, 10:1, or 5:1 fora target nucleic acid over a single nucleotide variant of the target nucleic acid. In some cases, a ribonucleoprotein may comprise a selectivity of at least 5:1 for a target nucleic acid over a single nucleotide variant of the target nucleic acid. Leveraging programmable nuclease selectivity, some methods described herein may detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample comprises 1 to 10,000, 100 to 8000, 400 to 6000, 500 to 5000, 1000 to 4000, or 2000 to 3000 target nucleic acids. In some cases, the method detects target nucleic acid present at least at one copy per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

Often, the target nucleic acid may be from 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. The target nucleic acid may also be 0.1% to 1% of the total nucleic acids in the sample. The target nucleic acid may be DNA or RNA. The target nucleic acid may be any amount less than 100% of the total nucleic acids in the sample. The target nucleic acid may be 100% of the total nucleic acids in the sample.

The target nucleic acid may be 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. Often, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. Often, the segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

A target nucleic acid may be an amplified nucleic acid of interest. The nucleic acid of interest may be any nucleic acid disclosed herein or from any sample as disclosed herein. The nucleic acid of interest may be an RNA that is reverse transcribed before amplification. The nucleic acid of interest may be amplified then the amplicons may be transcribed into RNA.

In some instances, compositions described herein exhibit indiscriminate trans-cleavage of ssRNA, enabling their use for detection of RNA in samples. In some cases, target ssRNA are generated from many nucleic acid templates (RNA) in order to achieve cleavage of the FQ reporter in the DETECTR platform. Certain programmable nucleases may be activated by ssRNA, upon which they may exhibit trans-cleavage of ssRNA and may, thereby, be used to cleave ssRNA FQ reporter molecules in the DETECTR system. These programmable nucleases may target ssRNA present in the sample, or generated and/or amplified from any number of nucleic acid templates (RNA). Described herein are reagents comprising a single stranded reporter nucleic acid comprising a detection moiety, wherein the reporter nucleic acid (e.g., the ssDNA-FQ reporter described herein) is capable of being cleaved by the programmable nuclease, upon generation and amplification of ssRNA from a nucleic acid template using the methods disclosed herein, thereby generating a first detectable signal.

In some instances, target nucleic acids comprise at least one nucleic acid comprising at least 50% sequence identity to the target nucleic acid or a portion thereof. Sometimes, the at least one nucleic acid comprises an amino acid sequence that is at least 60%, 70%, 75%, 80%, 85%, 90%, 91%, 92%, 93%, 94%, 95%, 96%, 97%, 98%, or 99% identical to an equal length portion of the target nucleic acid. Sometimes, the at least one nucleic acid comprises an amino acid sequence that is 100% identical to an equal length portion of the target nucleic acid. Sometimes, the amino acid sequence of the at least one nucleic acid is at least 60%, 70%, 75%, 80%, 85%, 90%, 91%, 92%, 93%, 94%, 95%, 96%, 97%, 98%, or 99% identical to the target nucleic acid. Sometimes, the target nucleic acid comprises an amino acid sequence that is less than 60%, 70%, 75%, 80%, 85%, 90%, 91%, 92%, 93%, 94%, 95%, 96%, 97%, 98%, or 99% identical to an equal length portion of the at least one nucleic acid.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid sequence at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid sequence. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid sequence. In some embodiments, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some embodiments, the target nucleic acid is not present in the sample.

A number of target nucleic acid populations are consistent with the methods and compositions disclosed herein. Some methods described herein may detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has 3 to 50, 5 to 40, or 10 to 25 target nucleic acid populations. In some cases, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations may be present at different concentrations or amounts in the sample.

In some embodiments, target nucleic acids may activate a programmable nuclease to initiate sequence-independent cleavage of a nucleic acid-based reporter (e.g., a reporter comprising an RNA sequence, or a reporter comprising DNA and RNA). For example, a programmable nuclease of the present disclosure is activated by a target nucleic acid to cleave reporters having an RNA (also referred to herein as an “RNA reporter”). Alternatively, a programmable nuclease of the present disclosure is activated by a target RNA to cleave reporters having an RNA (also referred to herein as a “RNA reporter”). The RNA reporter may comprise a single-stranded RNA labeled with a detection moiety or may be any RNA reporter as disclosed herein.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by a CRISPR/Cas system.

In some instances, the target nucleic acid as disclosed herein activates the programmable nuclease to initiate trans cleavage (also referred to as trans collateral cleavage) of catalytic oligonucleotides. In some instances, the target nucleic acid as disclosed herein activates the programmable nuclease to initiate trans cleavage (also referred to as trans collateral cleavage) of blocker oligonucleotides. In some embodiments, the target nucleic acid as disclosed herein activates the programmable nuclease to initiate trans cleavage (also referred to as trans collateral cleavage) of catalytic oligonucleotides, blocker oligonucleotides, or reporter molecules (e.g., a reporter molecule, such as an RNA reporter molecule, DNA reporter molecule, or a hybrid RNA-DNA reporter molecule), or any combination thereof. In some embodiments, the catalytic oligonucleotides comprise a cleavage site that is cleaved by the programmable nuclease upon binding to the target nucleic acid. In some embodiments, the blocker oligonucleotides comprise a cleavage site that is cleaved by the programmable nuclease upon binding to the target nucleic acid.

The methods, systems, compositions, reagents, and kits of the present disclosure can be used to process any a wide variety of samples to provide information about the status or condition of any subject or part of subject (e.g., organism, sample, human, animal). A status or condition of a subject can in some cases be a health-related condition, such as a disease in a subject (e.g., in a patient). Alternatively, the methods can determine if a substance, germ, pathogen, feature, or characteristic is present in a sample such as a material or substance (e.g., in an environmental sample or agricultural sample) which can potentially cause a state or condition such as a disease in a subject. For example, the samples described elsewhere herein can be used with the methods, compositions, reagents, enzymes, and kits disclosed herein for various applications such as diagnosis or prognosis of a disease listed anywhere herein, such RSV, sepsis, flu, or other diseases. In some examples, provided herein are reagent kits and point-of-care diagnostic tools.

These samples can comprise a target nucleic acid. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample can be obtained to test for presence of a disease, cancer, genetic disorder, or any mutation of interest. A biological sample from the individual can be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample can be dissociated or liquified prior to application to detection system of the present disclosure. A sample from an environment can be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system. Sometimes, the sample is contained in no more 20 μl. The sample, in some cases, is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl, preferably from 10 μL to 200 μL, or more preferably from 50 μL to 100 μL. Sometimes, the sample is contained in more than 500 μl.

In some instances, the target nucleic acid can be a single-stranded DNA or single-stranded RNA. The methods, reagents, enzymes, and kits disclosed herein can enable the direct detection of a DNA encoding a sequence of interest, in particular a single-stranded DNA encoding a sequence of interest, without transcribing the DNA into RNA, for example, by using an RNA polymerase, or without the need for amplification of the DNA and subsequence detection of the DNA amplicons. The methods, reagents, enzymes, and kits disclosed herein can enable the direct detection of a RNA encoding a sequence of interest, in particular a single-stranded RNA encoding a sequence of interest, without reverse transcribing the RNA into DNA, for example, or without the need for amplification of the RNA and subsequence detection of the RNA amplicons.

In some embodiments, the methods, reagents, enzymes, and kits disclosed herein can enable the detection of target nucleic acid that is an amplified nucleic acid of a nucleic acid of interest. In some embodiments, the target nucleic acid is a cDNA, genomic DNA, an amplicon of genomic DNA, a DNA amplicon, a DNA amplicon of an RNA, an RNA amplicon of a DNA, or an RNA amplicon. In some cases, the target nucleic acid that binds to the guide nucleic acid is a portion of a nucleic acid. A portion of a nucleic acid can encode a sequence from a genomic locus. A portion of a nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A portion of a nucleic acid can be from 10 to 90, from 20 to 80, from 30 to 70, or from 40 to 60 nucleotides in length. A portion of a nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can encode a sequence is reverse complementary to a guide nucleic acid sequence.

In some embodiments, the target nucleic acid is in a cell. In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample used for disease testing can comprise at least one target nucleic acid that can bind to a guide nucleic acid of the reagents described herein. The sample used for disease testing can comprise at least nucleic acid of interest that is amplified to produce a target nucleic acid that can bind to a guide nucleic acid of the reagents described herein. The nucleic acid of interest can comprise DNA, RNA, or a combination thereof.

The target nucleic acid (e.g., a target DNA) can be a portion of a nucleic acid from a virus or a bacterium or other agents responsible for a disease in the sample. The target nucleic acid can be a portion of a nucleic acid from a gene expressed in a cancer or genetic disorder in the sample. The target nucleic acid can comprise a genetic variation (e.g., a single nucleotide polymorphism), with respect to a standard sample, associated with a disease phenotype or disease predisposition. The target nucleic acid can be an amplicon of a portion of an RNA, can be a DNA, or can be a DNA amplicon from any organism in the sample.

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitides, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. Vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. Often the target nucleic acid comprises a sequence from a virus or a bacterium or other agents responsible for a disease that can be found in the sample. In some cases, the target nucleic acid is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitides, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

The sample used for cancer testing can comprise at least one target nucleic acid segment that can bind to a guide nucleic acid of the compositions described herein. The target nucleic acid segment, in some cases, is a portion of a nucleic acid from a gene with a mutation associated with cancer, from a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes for a cancer biomarker, such as a prostate cancer biomarker or non-small cell lung cancer. In some cases, the assay can be used to detect “hotspots” in target nucleic acids that can be predictive of lung cancer. In some cases, the target nucleic acid is a portion of a nucleic acid that is associated with a blood fever. In some cases, the target nucleic acid segment is a portion of a nucleic acid from a genomic locus, a transcribed mRNA, or a reverse transcribed cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1.

The sample used for genetic disorder testing can comprise at least one target nucleic acid segment that can bind to a guide nucleic acid of the compositions described herein. In some embodiments, the genetic disorder is hemophilia, sickle cell anemia, β-thalassemia, Duchene muscular dystrophy, severe combined immunodeficiency, or cystic fibrosis. The target nucleic acid segment, in some cases, is a portion of a nucleic acid from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid segment is a portion of a nucleic acid from a genomic locus, a transcribed mRNA, or a reverse transcribed cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

The sample used for genotyping testing can comprise at least one target nucleic acid segment that can bind to a guide nucleic acid of the compositions described herein. The target nucleic acid segment, in some cases, is a portion of a nucleic acid from a gene associated with a genotype.

The sample used for ancestral testing can comprise at least one target nucleic acid segment that can bind to a guide nucleic acid of the compositions described herein. The target nucleic acid segment, in some cases, is a portion of a nucleic acid from a gene associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method comprises obtaining a serum sample from a subject; and identifying a disease status of the subject. Often, the disease status is prostate disease status.

The sample can be used for testing for agricultural purposes. For example, a sample is any sample described herein, and is obtained from a subject (e.g., a plant) for use in identifying a disease status of a plant. The disease can be a disease that affects crops, such as a disease that affects rice, corn, wheat, or soy.

In some instances, the target nucleic acid is a single stranded nucleic acid. Alternatively or in combination, the target nucleic acid is a double stranded nucleic acid and is prepared into single stranded nucleic acids before or upon contacting the compositions. The target nucleic acid can be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the methods and compositions disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample has from 1 to 10,000, from 100 to 8000, from 400 to 6000, from 500 to 5000, from 1000 to 4000, or from 2000 to 3000 target nucleic acids. In some cases, the sample has from 100 to 9500, from 100 to 9000, from 100 to 8500, from 100 to 8000, from 100 to 7500, from 100 to 7000, from 100 to 6500, from 100 to 6000, from 100 to 5500, from 100 to 5000, from 250 to 9500, from 250 to 9000, from 250 to 8500, from 250 to 8000, from 250 to 7500, from 250 to 7000, from 250 to 6500, from 250 to 6000, from 250 to 5500, from 250 to 5000, from 2500 to 9500, from 2500 to 9000, from 2500 to 8500, from 2500 to 8000, from 2500 to 7500, from 2500 to 7000, from 2500 to 6500, from 2500 to 6000, from 2500 to 5500, or from 2500 to 5000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the methods and compositions disclosed herein. Some methods described herein can detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the sample has from 2 to 50, from 5 to 50, from 10 to 50, from 2 to 25, from 3 to 25, from 4 to 25, from 5 to 25, from 10 to 25, from 2 to 20, from 3 to 20, from 4 to 20, from 5 to 20, from 10 to 20, from 2 to 10, from 3 to 10, from 4 to 10, from 5 to 10, from 6 to 10, from 7 to 10, from 8 to 10, or from 9 to 10 target nucleic acid populations. In some cases, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

Additionally, a target nucleic acid can be amplified before binding to a guide nucleic acid, for example a crRNA of a CRISPR enzyme. This amplification can be PCR amplification or isothermal amplification. This nucleic acid amplification of the sample can improve at least one of sensitivity, specificity, or accuracy of the detection the target RNA. The compositions for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification is performed for from 5 to 60, from 10 to 60, from 15 to 60, from 30 to 60, from 45 to 60, from 1 to 45, from 5 to 45, from 10 to 45, from 30 to 45, from 1 to 30, from 5 to 30, from 10 to 30, from 15 to 30, from 1 to 15, from 5 to 15, or from 10 to 15 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C. In some cases, the nucleic acid amplification reaction is performed at a temperature of from 20° C. to 45° C., from 25° C. to 40° C., from 30° C. to 40° C., or from 35° C. to 40° C. In some cases, the nucleic acid amplification reaction are performed at a temperature of from 20° C. to 45° C., from 25° C. to 45° C., from 30° C. to 45° C., from 35° C. to 45° C., from 40° C. to 45° C., from 20° C. to 37° C., from 25° C. to 37° C., from 30° C. to 37° C., from 35° C. to 37° C., from 20° C. to 30° C., from 25° C. to 30° C., from 20° C. to 25° C., or from 22° C. to 25° C.

Any of the samples disclosed herein are consistent with the systems, assays, and programmable nucleases disclosed herein and can be used as a companion diagnostic with any of the diseases disclosed herein (e.g., RSV, sepsis, flu), or can be used in kits, point-of-care diagnostics, or over-the-counter diagnostics.

In some embodiments, the target nucleic acid sequence comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. A programmable nuclease of the disclosure (e.g., Cas14) may cleave the viral nucleic acid. In some embodiments, the target nucleic acid sequence comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

### Samples

The systems and methods of the present disclosure can be used to detect one or more target sequences or nucleic acids in one or more samples. The one or more samples can comprise one or more target sequences or nucleic acids for detection of an ailment, such as a disease, cancer, or genetic disorder, or genetic information, such as for phenotyping, genotyping, or determining ancestry and are compatible with the reagents and support mediums as described herein. Generally, a sample can be taken from any place where a nucleic acid can be found. Samples can be taken from an individual/human, a non-human animal, or a crop, or an environmental sample can be obtained to test for presence of a disease, virus, pathogen, cancer, genetic disorder, or any mutation or pathogen of interest. A biological sample can be blood, serum, plasma, lung fluid, exhaled breath condensate, saliva, spit, urine, stool, feces, mucus, lymph fluid, peritoneal, cerebrospinal fluid, amniotic fluid, breast milk, gastric secretions, bodily discharges, secretions from ulcers, pus, nasal secretions, sputum, pharyngeal exudates, urethral secretions/mucus, vaginal secretions/mucus, anal secretion/mucus, semen, tears, an exudate, an effusion, tissue fluid, interstitial fluid (e.g., tumor interstitial fluid), cyst fluid, tissue, or, in some instances, any combination thereof. A sample can be an aspirate of a bodily fluid from an animal (e.g., human, animals, livestock, pet, etc.) or plant. A tissue sample can be from any tissue that can be infected or affected by a pathogen (e.g., a wart, lung tissue, skin tissue, and the like). A tissue sample (e.g., from animals, plants, or humans) can be dissociated or liquified prior to application to detection system of the present disclosure. A sample can be from a plant (e.g., a crop, a hydroponically grown crop or plant, and/or house plant). Plant samples can include extracellular fluid, from tissue (e.g., root, leaves, stem, trunk etc.). A sample can be taken from the environment immediately surrounding a plant, such as hydroponic fluid/water, or soil. A sample from an environment can be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system. In some cases, the sample is contained in no more than about 200 nanoliters (nL). In some cases, the sample is contained in about 200 nL. In some cases, the sample is contained in a volume that is greater than about 200 nL and less than about 20 microliters (4). In some cases, the sample is contained in no more than 20 μl. In some cases, the sample is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl. In some cases, the sample is contained in from 1 μL to 500 μL, from 10 μL to 500 μL from 50 μL to 500 μL from 100 μL to 500 μL from 200 μL to 500 μL from 300 μL to 500 μL from 400 μL to 500 μL from 1 μL to 200 μL from 10 μL to 200 μL, from 50 μL to 200 μL, from 100 μL to 200 μL, from 1 μL to 100 μL, from 10 μL to 100 μL, from 50 μL to 100 μL, from 1 μL to 50 μL, from 10 μL to 50 μL, from 1 μL to 20 μL, from 10 μL to 20 μL, or from 1 μL to 10 μL. Sometimes, the sample is contained in more than 500 μl.

In some instances, the sample is taken from a single-cell eukaryotic organism; a plant or a plant cell; an algal cell; a fungal cell; an animal or an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample may comprise nucleic acids from a cell lysate from a eukaryotic cell, a mammalian cell, a human cell, a prokaryotic cell, or a plant cell. In some cases, the sample may comprise nucleic acids expressed from a cell.

The sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

The sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

The sample used for ancestral testing can comprise at least one target nucleic acid segment that can bind to a guide nucleic acid of the reagents described herein. The target nucleic acid segment, in some cases, is a portion of a nucleic acid from a gene associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method may comprise obtaining a serum sample from a subject; and identifying a disease status of the subject. Often, the disease status is prostate disease status. In any of the embodiments described herein, the device can be configured for asymptomatic, pre-symptomatic, and/or symptomatic diagnostic applications, irrespective of immunity. In any of the embodiments described herein, the device can be configured to perform one or more serological assays on a sample (e.g., a sample comprising blood).

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to respiratory viruses (e.g., SARS-CoV-2 (i.e., a virus that causes COVID-19), SARS-CoV-1, MERS-CoV, influenza, Adenovirus, Coronavirus HKU1, Coronavirus NL63, Coronavirus 229E, Coronavirus OC43, Severe Acute Respiratory Syndrome Coronavirus 2 (SARS-CoV-2), Human Metapneumovirus (hMPV), Human Rhinovirus (HRVs A, B, C), Human Enterovirus, Influenza A, Influenza A/H1, Influenza A/H2, Influenza A/H3, Influenza A/H4, Influenza A/H5, Influenza A/H6, Influenza A/H7, Influenza A/H8, Influenza A/H9, Influenza A/H10, Influenza A/H11, Influenza A/H12, Influenza A/H13, Influenza A/H14, Influenza A/H15, Influenza A/H16, Influenza A/H1-2009, Influenza A/N1 Influenza A/N2, Influenza A/N3, Influenza A/N4, Influenza A/N5, Influenza A/N6, Influenza A/N7, Influenza A/N8, Influenza A/N9, Influenza A/N10, Influenza A/N11, oseltamivir-resistant Influenza A, Influenza B, Influenza B—Victoria V1, Influenza B—Yamagata Y1, Influenza C, Parainfluenza Virus 1, Parainfluenza Virus 2, Parainfluenza Virus 3, Parainfluenza Virus 4, Respiratory Syncytial Virus A, Respiratory Syncytial Virus B) and respiratory bacteria (e.g. Bordetella parapertussis, Bordetella pertussis, Bordetella bronchiseptica, Bordetella holmesii, Chlamydia pneumoniae, Mycoplasma pneumoniae). Other viruses include human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitides, Chlamydia trachomatis, Chlamydia pneumoniae, Chlamydia psittaci, and Candida albicans. Pathogenic viruses include but are not limited to: respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g., warts (including genital, anal), herpes (including oral, genital, anal), molluscum contagiosum); hemmorhagic viral diseases (e.g. Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Klebsiella pneumoniae, Acinetobacter baumannii, Bacillus anthracis, Bordetella pertussis, Burkholderia cepacia, Corynebacterium diphtheriae, Coxiella burnetii, Streptococcus agalactiae, methicillin-resistant Staphylococcus aureus, Legionella longbeachae, Legionella pneumophila, Leptospira interrogans, Moraxella catarrhalis, Streptococcus pyogenes, Escherichia coli, Neisseria gonorrhoeae, Neisseria meningitidis, Neisseria elongate, Neisseria gonorrhoeae, Parechovirus, Pneumococcus, Pneumocystis jirovecii, Cryptococcus neoformans, Histoplasma capsulatum, Haemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T Vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. Often the target nucleic acid may comprise a sequence from a virus or a bacterium or other agents responsible for a disease that can be found in the sample. In some cases, the target nucleic acid is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitides, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Staphylococcus epidermidis, Legionella pneumophila, Streptococcus pyogenes, Streptococcus salivarius, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), Alphacoronavirus, Betacoronavirus, Sarbecovirus, SARS-related virus, Gammacoronavirus, Deltacoronavirus, M. genitalium, T. vaginalis, varicella-zoster virus, hepatitis B virus, hepatitis C virus, measles virus, human adenovirus (type A, B, C, D, E, F, G), human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Human Bocavirus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. SARS-CoV-2 Variants include Coronavirus HKU1, Coronavirus NL63, Coronavirus 229E, Coronavirus OC43, SARS-CoV-2 85Δ, SARS-CoV-2 T1001I, SARS-CoV-2 3675-3677Δ, SARS-CoV-2 P4715L, SARS-CoV-2 S5360L, SARS-CoV-2 69-70Δ, SARS-CoV-2 Tyr144fs, SARS-CoV-2 242-244Δ, SARS-CoV-2 Y453F, SARS-CoV-2 S477N, SARS-CoV-2 E848K, SARS-CoV-2 N501Y, SARS-CoV-2 D614G, SARS-CoV-2 P681R, SARS-CoV-2 P681H, SARS-CoV-2 L21F, SARS-CoV-2 Q27Stop, SARS-CoV-2 M1fs, and SARS-CoV-2 R203fs. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some instances, the target sequence is a portion of a nucleic acid from a subject having cancer. The cancer may be a solid cancer (tumor). The cancer may be a blood cell cancer, including leukemias and lymphomas. Non-limiting types of cancer that could be treated with such methods and compositions include colon cancer, rectal cancer, renal-cell carcinoma, liver cancer, bladder cancer, cancer of the kidney or ureter, lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, thyroid cancer. The cancer may be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL).

In some instances, the target sequence is a portion of a nucleic acid from a cancer cell. A cancer cell may be a cell harboring one or more mutations that results in unchecked proliferation of the cancer cell. Such mutations are known in the art. Non-limiting examples of antigens are ADRB3, AKAP-4, ALK, Androgen receptor, B7H3, BCMA, BORIS, BST2, CAIX, CD 179a, CD123, CD171, CD19, CD20, CD22, CD24, CD30, CD300LF, CD33, CD38, CD44v6, CD72, CD79a, CD79b, CD97, CEA, CLDN6, CLEC12A, CLL-1, CS-1, CXORF61, CYP1B1, Cyclin B 1, E7, EGFR, EGFRvIII, ELF2M, EMR2, EPCAM, ERBB2 (Her2/neu), ERG (TMPRSS2 ETS fusion gene), ETV6-AML, EphA2, Ephrin B2, FAP, FCAR, FCRL5, FLT3, Folate receptor alpha, Folate receptor beta, Fos-related antigen 1, Fucosyl GM1, GD2, GD3, GM3, GPC3, GPR20, GPRC5D, GloboH, HAVCR1, HMWMAA, HPV E6, IGF-I receptor, IL-13Ra2, IL-1 1Ra, KIT, LAGE-1a, LAIR1, LCK, LILRA2, LMP2, LY6K, LY75, LewisY, MAD-CT-1, MAD-CT-2, MAGE A1, MAGE-A1, ML-IAP, MUC1, MYCN, MelanA/MART1, Mesothelin, NA17, NCAM, NY-BR-1, NY-ESO-1, OR51E2, OY-TES 1, PANX3, PAP, PAX3, PAX5, PCTA-1/Galectin 8, PDGFR-beta, PLAC1, PRSS21, PSCA, PSMA, Polysialic acid, Prostase, RAGE-1, ROR1, RU1, RU2, Ras mutant, RhoC, SART3, SSEA-4, SSX2, TAG72, TARP, TEM1/CD248, TEM7R, TGS5, TRP-2, TSHR, Tie 2, Tn Ag, UPK2, VEGFR2, WT1, XAGE1, and IGLL1.

In some cases, the target sequence is a portion of a nucleic acid from a control gene in a sample. In some embodiments, the control gene is an endogenous control. The endogenous control may include human 18S rRNA, human GAPDH, human HPRT1, human GUSB, human RNase P, MS2 bacteriophage, or any other control sequence of interest within the sample.

### Mutations

In some instances, target nucleic acids comprise a mutation. In some instances, a sequence comprising a mutation may be modified to a wildtype sequence with a composition, system or method described herein. In some instances, a sequence comprising a mutation may be detected with a composition, system or method described herein. The mutation may be a mutation of 1, 2, 3, 4, 5, 6, 7, 8, 9, 10, 11, 12, 13, 14, 15, 16, 17, 18, 19, 20 or more nucleotides. Non-limiting examples of mutations are insertion-deletion (indel), single nucleotide polymorphism (SNP), and frameshift mutations. In some instances, guide nucleic acids described herein hybridize to a region of the target nucleic acid comprising the mutation. The mutation may be located in a non-coding region or a coding region of a gene.

In some instances, target nucleic acids comprise a mutation, wherein the mutation is a SNP. The single nucleotide mutation or SNP may be associated with a phenotype of the sample or a phenotype of the organism from which the sample was taken. The SNP, in some cases, is associated with altered phenotype from wild type phenotype. The SNP may be a synonymous substitution or a nonsynonymous substitution. The nonsynonymous substitution may be a missense substitution or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a maycer cell.

In some instances, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. The mutation may be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

### Multiplexing

The systems, devices, and methods described herein can be multiplexed in a number of ways. Multiplexing may include assaying for two or more target nucleic acids in a sample. Multiplexing can be spatial multiplexing wherein multiple different target nucleic acids are detected from the same sample at the same time, but the reactions are spatially separated. Often, the multiple target nucleic acids are detected using the same programmable nuclease, but different guide nucleic acids. The multiple target nucleic acids sometimes are detected using the different programmable nucleases. Sometimes, multiplexing can be single reaction multiplexing wherein multiple different target acids are detected in a single reaction volume. Often, at least two different programmable nucleases are used in single reaction multiplexing. For example, multiplexing can be enabled by immobilization of multiple categories of reporters within a device, to enable detection of multiple target nucleic acids. Multiplexing allows for detection of multiple target nucleic acids in one kit or system. In some cases, the multiple target nucleic acids comprise different target nucleic acids to a virus. In some cases, the multiple target nucleic acids comprise different target nucleic acids associated with at least a first disease and a second disease. Multiplexing for one disease can increase at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. For example, multiplexing methods may comprise a single assay for a microorganism species using a first programmable nuclease and an antibiotic resistance pattern in a microorganism using a second programmable nuclease. Sometimes, multiplexing allows for discrimination between multiple target nucleic acids of different influenza strains, for example, influenza A and influenza B. Often, multiplexing allows for discrimination between multiple target nucleic acids, such as target nucleic acids that comprise different genotypes, for example, for a wild-type genotype and for a mutant (e.g., SNP) genotype. Multiplexing for multiple viral infections can provide the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Furthermore, signals from multiplexing can be quantified. For example, a method of quantification for a disease panel comprises assaying for a plurality of unique target nucleic acids in a plurality of aliquots from a sample, assaying for a control nucleic acid control in another aliquot of the sample, and quantifying a plurality of signals of the plurality of unique target nucleic acids by measuring signals produced by cleavage of reporters compared to the signal produced in the second aliquot. Often the plurality of unique target nucleic acids are from a plurality of viruses in the sample. Sometimes the quantification of a signal of the plurality correlates with a concentration of a unique target nucleic acid of the plurality for the unique target nucleic acid of the plurality that produced the signal of the plurality. The disease panel can be for any disease.

In some cases, the combination of a guide nucleic acid, a programmable nuclease, and a single stranded reporter configured to detect one target nucleic acid is provided in its own reagent chamber or its own support medium. In this case, multiple reagent chambers or support mediums are provided, where each reagent chamber is designed to detect one target nucleic acid. In some cases, multiple different target nucleic acids may be detected in the same chamber or support medium.

In some instances, the multiplexed devices and methods detect at least 2 different target nucleic acids in a single reaction. In some instances, the multiplexed devices and methods detect at least 3 different target nucleic acids in a single reaction. In some instances, the multiplexed devices and methods detect at least 4 different target nucleic acids in a single reaction. In some instances, the multiplexed devices and methods detect at least 5 different target nucleic acids in a single reaction. In some cases, the multiplexed devices and methods detect at least 6, 7, 8, 9, or 10 different target nucleic acids in a single reaction.

### Buffers

The compositions and methods of use thereof described herein can also include buffers, which are compatible with the methods and compositions disclosed herein. These buffers can be used for detection of an ailment, such as a disease, cancer, or genetic disorder, or genetic information, such as for phenotyping, genotyping, or determining ancestry. As described herein, nucleic acid sequences can be detected using a programmable nuclease, guide nucleic acid, catalytic oligonucleotide, and blocker oligonucleotide as disclosed herein. Additionally, detection by a programmable nuclease that cleaves reporter RNA molecules allows for multiplexing with other programmable nucleases, such as a programmable nuclease that can cleave DNA reporters (e.g., Type V CRISPR enzyme). The methods described herein can also include the use of buffers, which are compatible with the methods disclosed herein.

The buffers described herein are compatible for use in the devices described herein (e.g., pneumatic valve devices, sliding valve devices, rotating valve devices, and lateral flow devices) and may be used in conjunction with compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether the target nucleic acid is in the sample (e.g., DETECTR reactions). These buffers are compatible with the other reagents, samples, and support mediums as described herein for detection of an ailment, such as a disease, cancer, or genetic disorder, or genetic information, such as for phenotyping, genotyping, or determining ancestry. The methods described herein can also include the use of buffers, which are compatible with the methods disclosed herein. In some cases, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some instances, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, 7 to 9, 7 to 9.5, 6.5 to 8, 6.5 to 9, 6.5 to 9.5, 7.5 to 8.5, 7.5 to 9, 7.5 to 9.5, or 9.5 to 10.5. The pH of the solution may also be at least about 6.0, at least about 6.5, at least about 7.0, at least about 7.5, at least about 8.0, at least about 8.5, or at least about 9. In some cases, the pH is at least about 6. In some cases, the pH is at least about 6.5. In some cases, the pH is at least about 7. In some cases, the pH is at least about 7.5. In some cases, the pH is at least about 8. In some cases, the pH is at least about 8.5. In some cases, the pH is at least about 9.

For example, a buffer comprises 20 mM HEPES pH 6.8, 50 mM KCl, 5 mM MgCl₂, and 5% glycerol. In some instances the buffer comprises from 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer can comprise 0 to 25, 0 to 20, 0 to 10, 0 to 5, 5 to 10, 5 to 15, 5 to 20, 5 to 25, 5 to 30% glycerol. The buffer can comprise 0 to 30, 2 to 25, or 10 to 20% glycerol.

As another example, a buffer comprises 100 mM Imidazole pH 7.5; 250 mM KCl, 25 mM MgCl₂, 50 ug/mL BSA, 0.05% Igepal Ca-630, and 25% Glycerol. In some instances the buffer comprises 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Imidazole pH 7.5. In some instances, the buffer comprises 100 to 250, 100 to 200, or 150 to 200 mM Imdazole pH 7.5. The buffer can comprise 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer, in some instances, comprises 0 to 100, 0 to 75, 0 to 50, 0 to 25, 0 to 20, 0 to 10, 0 to 5, 5 to 50, 5 to 75, 5 to 100, 10 to 20, 10 to 50, 10 to 75, 10 to 100, 25 to 50, 25 to 75 25 to 100, 50 to 75, or 50 to 100 ug/mL BSA. In some instances, the buffer comprises 0 to 1, 0 to 0.5, 0 to 0.25, 0 to 0.01, 0 to 0.05, 0 to 0.025, 0 to 0.01, 0.01 to 0.025, 0.01 to 0.05, 0.01 to 0.1, 0.01 to 0.25, 0.01, to 0.5, 0.01 to 1, 0.025 to 0.05, 0.025 to 0.1, 0.025, to 0.5, 0.025 to 1, 0.05 to 0.1, 0.05 to 0.25, 0.05 to 0.5, 0.05 to 0.75, 0.05 to 1, 0.1 to 0.25, 0.1 to 0.5, or 0.1 to 1% Igepal Ca-630. The buffer can comprise 0 to 25, 0 to 20, 0 to 10, 0 to 5, 5 to 10, 5 to 15, 5 to 20, 5 to 25, 5 to 30% glycerol. The buffer can comprise 0 to 30, 2 to 25, or 10 to 20% glycerol.

### Stability

Present in this disclosure are stable compositions for use in the methods of detection as described herein. The compositions described herein can be stable in various storage conditions including refrigerated, ambient, and accelerated conditions. The stability can be measured for the compositions themselves, the components of the compositions, or the compositions present on the support medium.

In some embodiments, stable as used herein refers to a compositions having about 5% w/w or less total impurities at the end of a given storage period. Stability can be assessed by HPLC or any other known testing method. The stable compositions can have about 10% w/w, about 5% w/w, about 4% w/w, about 3% w/w, about 2% w/w, about 1% w/w, or about 0.5% w/w total impurities at the end of a given storage period. The stable compositions can have from 0.5% w/w to 10% w/w, from 1% w/w to 8% w/w, from 2% w/w to 7% w/w, or from 3% w/w to 5% w/w total impurities at the end of a given storage period.

In some embodiments, stable as used herein refers to a compositions having about 10% or less loss of detection activity at the end of a given storage period and at a given storage condition. Detection activity can be assessed by known positive sample using a known method. Alternatively or in combination, detection activity can be assessed by the sensitivity, accuracy, or specificity. In some embodiments, the stable compositions can have about 10%, about 9%, about 8%, about 7%, about 6%, about 5%, about 4%, about 3%, about 2%, about 1%, or about 0.5% loss of detection activity at the end of a given storage period. In some embodiments, the stable compositions can have from about 0.5% to 10%, from about 1% to 8%, from 2% to 7%, or from 3% to 5% loss of detection activity at the end of a given storage period.

In some examples, the stable composition has zero loss of detection activity at the end of a given storage period and at a given storage condition. The given storage condition can comprise humidity of equal to or less than 10%, 20%, 30%, 40%, 50%, 60%, 70%, 80%, 90%, or 100% relative humidity. The controlled storage environment can comprise humidity from 0% to 50% relative humidity, from 0% to 40% relative humidity, from 0% to 30% relative humidity, from 0% to 20% relative humidity, or from 0% to 10% relative humidity. The controlled storage environment can comprise humidity from 10% to 80%, from 10% to 70%, from 10% to 60%, from 20% to 50%, from 20% to 40%, or from 20% to 30% relative humidity. The controlled storage environment can comprise temperatures of about −100° C., about −80° C., about −20° C., about 4° C., about 25° C. (room temperature), or about 40° C. The controlled storage environment can comprise temperatures from −80° C. to 25° C., or from −100° C. to 40° C. The controlled storage environment can comprise temperatures from −20° C. to 40° C., from −20° C. to 4° C., or from 4° C. to 40° C. The controlled storage environment can protect the system or kit from light or from mechanical damage. The controlled storage environment can be sterile or aseptic or maintain the sterility of the light conduit. The controlled storage environment can be aseptic or sterile.

### Certain Methods of Detection

Provided herein are methods of nucleic acid detection using the compositions as described herein. In some embodiments, a method comprises: (a) contacting a sample to a composition comprising a guide nucleic acid that hybridizes to a segment of a target nucleic acid, a programmable nuclease, a signal amplifier (e.g., a catalytic oligonucleotide), and a reporter molecule; and (b) assaying for a signal produced by cleavage of the reporter molecule. In some embodiments, a method comprises: (a) contacting a sample to a composition comprising a guide nucleic acid that hybridizes to a segment of a target nucleic acid, a programmable nuclease, a signal amplifier (e.g., a catalytic oligonucleotide), a blocker oligonucleotide, and a reporter molecule; and (b) assaying for a signal produced by cleavage of the reporter molecule. In some embodiments, the composition comprises a plurality of reporter molecules.

In some embodiments, a method comprises: (a) contacting a sample to a composition comprising a guide nucleic acid that hybridizes to a segment of a target nucleic acid, a programmable nuclease, a first signal amplifier (e.g., a first catalytic oligonucleotide), a second signal amplifier (e.g., a second catalytic oligonucleotide), a first blocker oligonucleotide, a second blocker oligonucleotide, and a reporter molecule; and (b) assaying for a signal produced by cleavage of the reporter molecule. In some embodiments, the composition comprises a plurality of reporter molecules.

In some embodiments, a method comprises: (a) contacting a sample to a composition comprising a guide nucleic acid that hybridizes to a segment of a target nucleic acid, a programmable nuclease, a signal amplifier (e.g., a catalytic oligonucleotide), and a reporter molecule; (b) activating the signal amplifier (e.g., cleaving the catalytic oligonucleotide) and cleaving the reporter molecule by the programmable nuclease bound to the guide nucleic acid upon binding to the target nucleic acid; (c) cleaving the reporter molecule by the signal amplifier (e.g., catalytic oligonucleotide) upon cleavage by the programmable nuclease; and (d) assaying for a signal produced by cleavage of the reporter molecule. In some embodiments, the catalytic oligonucleotide is circular in step (a), and when cleaved in step (b), forms a secondary structure that has cleavage activity. In some embodiments, the composition comprises a plurality of reporter molecules.

In some embodiments, a method comprises: (a) contacting a sample to a composition comprising a guide nucleic acid that hybridizes to a segment of a target nucleic acid, a programmable nuclease, a signal amplifier (e.g., a catalytic oligonucleotide), a blocker oligonucleotide, and a reporter molecule; (b) cleaving the blocker oligonucleotide by the programmable nuclease bound to the guide nucleic acid upon binding to the target nucleic acid; (c) cleaving the reporter molecule by the signal amplifier (e.g., catalytic oligonucleotide) upon cleavage of the blocker oligonucleotide by the programmable nuclease; and (d) assaying for a signal produced by cleavage of the reporter molecule. In some embodiments, the signal amplifier (e.g., catalytic oligonucleotide) is bound to the blocker oligonucleotide in step (a), and when the blocker oligonucleotide is cleaved in step (b), the signal amplifier (e.g., catalytic oligonucleotide) is capable of forming a secondary structure that has cleavage activity. In some embodiments, there are a plurality of signal amplifiers (e.g., catalytic oligonucleotides) bound to the blocker oligonucleotides in step (a), and when the blocker oligonucleotide is cleaved in step (b), the signal amplifier (e.g., catalytic oligonucleotide) is capable of forming a secondary structure that has cleavage activity for cleaving both a report molecule and blocker oligonucleotides bound to signal amplifiers (e.g., catalytic oligonucleotides). In some embodiments, the composition comprises a plurality of reporter molecules.

In some embodiments, a method comprises: (a) contacting a sample to a composition comprising a guide nucleic acid that hybridizes to a segment of a target nucleic acid, a programmable nuclease, a first signal amplifier (e.g., a first catalytic oligonucleotide), a second signal amplifier (e.g., a second catalytic oligonucleotide), a first blocker oligonucleotide, a second blocker oligonucleotide, and a reporter molecule; (b) cleaving the first blocker oligonucleotide by the programmable nuclease bound to the guide nucleic acid upon binding to the target nucleic acid; (c) cleaving the reporter molecule by the first signal amplifier (e.g., first catalytic oligonucleotide) upon cleavage of the first blocker oligonucleotide by the programmable nuclease; (d) cleaving the second blocker oligonucleotide by the first signal amplifier (e.g., first catalytic oligonucleotide) upon cleavage of the first blocker oligonucleotide by the programmable nuclease; (e) cleaving the first blocker by the second signal amplifier (e.g., second catalytic oligonucleotide); and (d) assaying for a signal produced by cleavage of the reporter molecule. In some embodiments, the first signal amplifier (e.g., first catalytic oligonucleotide) is bound to the first blocker oligonucleotide and the second signal amplifier (e.g., second catalytic oligonucleotide) is bound to the second blocker oligonucleotide in step (a), and when the first blocker oligonucleotide is cleaved in step (b), the first signal amplifier (e.g., first catalytic oligonucleotide) is capable of forming a secondary structure that has cleavage activity. In some embodiments, the first signal amplifier (e.g., first catalytic oligonucleotide) is bound to the first blocker oligonucleotide and the second signal amplifier (e.g., second catalytic oligonucleotide) is bound to the second blocker oligonucleotide in step (a), and when the second blocker oligonucleotide is cleaved in step (c), the second signal amplifier (e.g., second catalytic oligonucleotide) is capable of forming a secondary structure that has cleavage activity. In some embodiments, the composition comprises a plurality of reporter molecules. In some embodiments, the composition comprises a plurality of first signal amplifiers (e.g., first catalytic oligonucleotides), a plurality of second signal amplifiers (e.g., second catalytic oligonucleotides), a plurality of first blocker oligonucleotides, and a plurality of second blocker oligonucleotides.

In the methods as described herein, a reporter molecule can be cleaved by a programmable nuclease. A reporter molecule can be cleaved by a signal amplifier (e.g., catalytic oligonucleotide). A reporter molecule can be cleaved by a first signal amplifier (e.g., first catalytic oligonucleotide). In some embodiments, a signal amplifier (e.g., catalytic oligonucleotide) is cleaved by a programmable nuclease. In some embodiments, a blocker oligonucleotide is cleaved by a programmable nuclease. In some embodiments, a blocker oligonucleotide is cleaved by a signal amplifier (e.g., catalytic oligonucleotide). In some embodiments, a first blocker is cleaved by a programmable nuclease. In some embodiments, a first blocker is cleaved by a second signal amplifier (e.g., second catalytic oligonucleotide). In some embodiments, a second blocker is cleaved by a first signal amplifier (e.g., first catalytic oligonucleotide).

In the methods as described herein, binding the guide nucleic acid to the target nucleic acid can activate a trans-cleavage activity of the programmable nuclease. In some cases, the trans-cleavage activity of the programmable nuclease can be non-specific. For example, in some cases, the programmable nuclease can nearby nucleic acid sequences indiscriminately and/or non-specifically. The activated programmable nuclease can cleave the reporter molecule which can generate a signal. The signal can be a measurable signal. The signal can be a fluorescent signal. The fluorescent signal can be measured using various measurement techniques (e.g., fluorometric measurement) and can be indicative of detection of the target nucleic acid molecule (e.g., its binding to the guide nucleic acid molecule).

In the methods as described herein, a signal amplifier comprising a catalytic oligonucleotide can be activated (e.g., by cleaving a circular form of the catalytic oligonucleotide or cleaving the blocker oligonucleotide that inhibits the catalytic oligonucleotide from forming a secondary structure that has cleavage activity) and configured to cleave a reporter molecule (e.g., a reporter that is the same as or similar to the reporter cleaved by the programmable nuclease or a different reporter), thereby generating a signal. The signal generated at this stage can be the same as the signal generated due to the cleavage of the reporter molecule by the programmable nuclease, and therefore can be intensified. In some cases, the signal generated due to cleavage of a reporter by the catalytic oligonucleotide can be different from the signal generated due to cleavage of the reporter molecule by the programmable nuclease.

The programmable nuclease can be an RNA targeting nuclease. In some examples, the programmable nuclease can be Cas13. The reporter molecule can comprise a moiety which can release the signal upon cleavage from the reporter molecule. The signal can be a fluorescent signal. In some examples, the reporter molecule can comprise a hairpin structure. In some examples, the reporter molecule can comprise a linear structure.

In some examples, the method further comprises providing more than one reporter molecules, such as 2, 3, 4, 5, 6, 7, 8, 9, 10, or more different reporter molecules. Multiple copies each reporter molecule can be present in the sample, for example, each reporter can be provided at a predefined concentration and/or ratio compared to other composition compounds.

In some embodiments, upon hybridizing the guide nucleic acid to the segment of the target nucleic acid, the programmable nuclease can cleave a reporter molecule thereby generating a signal. Further, a signal amplifier (e.g., a catalytic oligonucleotide) in the sample/composition can be activated according to the descriptions provided elsewhere herein. The signal amplifier (e.g., catalytic oligonucleotide) can cleave a reporter molecule, thereby generating a signal. In some cases, the signal amplifier (e.g., catalytic oligonucleotide) can further cleave other signal amplifier (e.g., catalytic oligonucleotide) that in an inactive (e.g., circular form) or cleave blocker oligonucleotides, thereby producing more signal amplifiers (e.g., catalytic oligonucleotides) with cleavage activity that are able cleave the reporter molecules.

In some embodiments, upon hybridizing the guide nucleic acid to the segment of the target nucleic acid, the programmable nuclease can cleave a reporter molecule thereby generating a signal. Further, a first catalytic oligonucleotide in the sample/composition can be activated according to the descriptions provided elsewhere herein. The first catalytic oligonucleotide can cleave a reporter molecule, thereby generating a signal. In some cases, the first catalytic oligonucleotide can further cleave second blocker oligonucleotides to activate second catalytic oligonucleotides, which can then cleave first blocker oligonucleotides, thereby producing more first catalytic oligonucleotides with cleavage activity that are able cleave the reporter molecules.

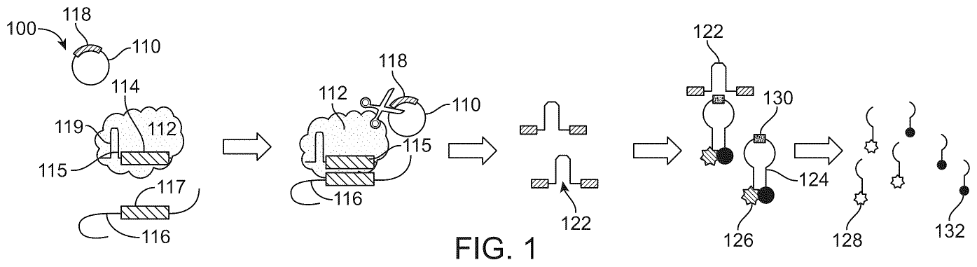

An example of the compositions and methods provided herein is shown in FIG. 1. The composition shown in FIG. 1 can comprise a signal amplifier 100 comprising a catalytic oligonucleotide 110. The composition may further comprise a programmable nuclease 112, a guide nucleic acid 115, and a target nucleic acid 116. Such as shown in FIG. 1, in some examples, the programmable nuclease and the guide nucleic acid can be bound, for example in a complex. For example, the programmable nuclease and the guide nucleic acid can be provided separately in the composition and be subjected to conditions sufficient for them to complex with one another. In some cases, this can be referred to as a complexing reaction. Alternatively, or in combination, the programmable nuclease and the guide nucleic acid can be complexed prior to being added to the composition. In some cases, the programmable nuclease, the guide nucleic acid, and/or a complex comprising both can be present in the composition.

The guide nucleic acid 115 can comprise a sequence 114 which can comprise a region that is complementary to a target sequence 117 of the target nucleic acid and a scaffold sequence 119 that binds to the programmable nuclease 112. For example, sequence 114 of the guide nucleic acid 115 can be configured to hybridize to the target sequence 117 of the target nucleic acid 116. In some cases, sequence 114 can be the same or substantially the same as sequence 117. The programmable nuclease (e.g., a Cas enzyme, such as Cas13) 112 can cleave the circular form of the catalytic oligonucleotide 110. For example, upon hybridization of the guide nucleic acid 115 to the target nucleic acid 116, trans-cleavage can be activated in the programmable nuclease. The programmable nuclease 112 can then cleave the circular form of the catalytic oligonucleotide 110 and thereby activate it, for example by allowing the catalytic oligonucleotide to form a secondary structure capable of having catalytic activity, e.g., binding and cleavage activity. The catalytic oligonucleotide 110 can comprise a circular structure and a segment 118 of a ribonucleic acid (RNA) molecule can be cleaved by the programmable nuclease, such as shown in the example of FIG. 1. Additionally, the programmable nuclease can cleave a reporter molecule.

In some embodiments, the signal amplifier 100 may comprise a circular catalytic oligonucleotide 110. The programmable nuclease 112 can cleave the RNA segment 118 in the circular catalytic oligonucleotide 110. Upon cleavage, the catalytic oligonucleotide can be modified to a linearized oligonucleotide 122 with catalytic activity, such as binding and cleavage activity.

With continued reference to FIG. 1, upon hybridization of the guide nucleic acid 115 to the target nucleic acid 116, the programmable nuclease can cleave a reporter molecule. Cleavage of the reporter molecule such as reporter 124 or another reporter molecule can generate a detectable signal.

The activated (e.g., linearized) catalytic oligonucleotide 122 (e.g., DNAzyme) can cleave a reporter molecule (e.g., reporter 124). In some examples, the reporter molecule 124 can comprise a secondary structure, such as a hairpin structure. In some examples, the reporter molecule 124 can comprise a linear structure. The reporter molecule can comprise a sequence 130 which can be recognized and targeted by the catalytic oligonucleotide 122 and/or the programmable nuclease 112. For example, the catalytic oligonucleotide 122 (e.g., DNAzyme) can bind to sequence 130 of the reporter molecule 124 and cleave it from the site of binding. The cleavage of the reporter molecule 124 can be used to activate quenched fluorescent reporter molecules, generate signals that can be visualized on a lateral flow strip, and/or other readout or detection methods.

In some examples, the reporter molecule 124 can further comprise a moiety 126 (e.g., at one end) which can release a fluorescent signal upon cleavage of the cleavage sequence 130. For example, moiety 126 can comprise or be a fluorophore or a fluorogenic substrate. The fluorescent activity of moiety 126 can be dampened, quenched, and/or otherwise decreased, halted or inactivated, for example, as long as the two sequences (e.g., including sequence 132) of the reporter 124 are bound to one another, for example through the cleavage sequence 130 or at the cleavage site 130. Upon cleavage of the cleavage sequence 130 or the cleavage site by the catalytic 122 and/or by the programmable nuclease 112, moiety 126 can be released (e.g., in form of released moiety 128) in the composition/sample and can generate a detectable and/or measurable signal (e.g., fluorescent signal). Moiety 128 can be a fluorophore which can be free-floating in the composition upon and/or after cleavage. In some cases, the combination of the signals generated by cleavage of the reporter molecules (e.g., by the programmable nuclease and/or the catalytic oligonucleotide) can be measured. Stated a different way, in some cases, the signal generated due to cleavage of the reporter molecule by the programmable nuclease can be intensified by the cleavage of the reporter molecule by the catalytic oligonucleotide, and thereby can enhance the sensitivity of the assay compared to an assay which does not include the catalytic oligonucleotide. This method and composition can facilitate detecting target nucleic acid molecules which can be present at lower concentrations in a sample, and/or which have not been amplified, for example by a polymerase chain reaction (PCR). In some examples, the compositions and methods provided herein can comprise performing a sensitive assay and can be performed without pre-amplification of the target nucleic acid.

In some examples, the catalytic oligonucleotide can be configured to bind to a blocker oligonucleotide that is bound to additional catalytic oligonucleotides whose catalytic activity is inhibited by binding to a blocker oligonucleotide, thereby generating larger quantities of the catalytic oligonucleotide that can cleave the reporter molecules. Examples of this are described and illustrated in further detail elsewhere herein.

In some examples, the methods can comprise providing a circular DNAzyme precursor which can comprise RNA bases. In some cases, when the RNA bases can be cleaved, the DNAzymes can adopt a conformation or structure such as a secondary structure it can need to become active. The activated DNAzyme can cleave a reporter molecule, which can comprise RNA bases recognizable by the DNAzyme. The reporter can comprise a fluorophore and a fluorescent quencher. The reporter molecule can be cleaved by a DNAzyme and/or a Cas enzyme, and can generate a fluorescent signal.

In some cases, the method provided herein can comprise two or more signal generation steps. The first can be generated as a result of a nuclease (e.g., Cas enzyme, such as Cas13) recognizing its target nucleic acid which can activate a trans collateral cleavage and subsequent cleavage of the reporter molecule. The second signal generation step, also referred to herein as a signal amplification step, can be achieved by an active signal amplifier (e.g., DNAzyme) configured to cleave one or more (e.g., multiple) reporter substrate molecules, for example, to generate fluorescent signals. The methods of the present disclosure can be performed in a variety of ways. For example, a CRISPR-based diagnostics approach can be coupled to a signal amplifier system in a variety of ways. In some examples, a nuclease, such as a Cas enzyme can activate a catalytic oligonucleotide molecule such as a DNAzyme molecule. Alternatively, or in addition, the nuclease (e.g., a Cas enzyme) can initiate an autocatalytic cycle. For example, upon initial detection of the target nucleic acid by the nuclease (e.g., thje Cas enzyme), multiple DNAzymes can be used to activate each other and one or more fluorescent reporters of the same and/or of different times. Such methods are described in further detail elsewhere herein.

Another example of the methods and compositions of the present disclosure is provided in FIG. 2. The composition shown in FIG. 2 comprises a signal amplifier 210, a programmable nuclease 112, a guide nucleic acid 115 comprising a guide sequence 114, and a target nucleic acid 116 comprising a target sequence 117. The signal amplifier 210 may comprise a blocker oligonucleotide 212 configured to maintain the signal amplifier 210 in an inactive state until removal thereof by the programmable nuclease, activated signal amplifier, and/or other component of the signal amplification cascade and feedback system. In some embodiments, the signal amplifier 210 may comprise a catalytic oligonucleotide 211 bound to a blocker oligonucleotide 212. In this example, the catalytic oligonucleotide is a DNAzyme inactivated by a blocker oligonucleotide 212 which forces it into an inactive circular or semi-circular structure. Stated a different way, the catalytic oligonucleotide can be in an oligonucleotide complex in which the catalytic oligonucleotide (e.g., oligonucleotide 211) is bound to a blocker oligonucleotide (e.g., oligonucleotide 212). The activity of the catalytic oligonucleotide 211 can be blocked by the blocker oligonucleotide 212, for example, as long as it is bound to the blocker oligonucleotide 212. The blocker oligonucleotide 212 can comprise a cleavage sequence 214. In some examples, the cleavage sequence 214 can comprise a segment of an RNA molecule which can be configured to be recognized by and/or cleaved by a programmable nuclease (e.g., Cas13). Upon hybridization of the guide nucleic acid 115 to the target nucleic acid 116, trans-cleavage activity can be initiated in the programmable nuclease 112. The programmable nuclease 112 can cleave a reporter molecule (e.g., reporter 220 or another reporter) and generate a measurable signal. In some cases, this event can be referred to as the first signal amplification. The measurable signal can be a fluorescent signal.

The programmable nuclease 112 can proceed to cleave the blocker oligonucleotide cleavage sequence 214 (e.g., segment of RNA) and thereby modify the oligonucleotide complex such that the cleaved blocker 218 releases the inactive catalytic oligonucleotide 211. The catalytic oligonucleotide is then able form an unblocked secondary structure that has catalytic activity 216 (e.g., active DNAzyme which does not comprise the blocker oligonucleotide sequence). The active catalytic oligonucleotide 216 (e.g., active DNAzyme) can bind to a reporter molecule 220 (e.g., reporter 220). The reporter molecule 220 can comprise two or more moieties or sequences (e.g., including sequence 227) bound or conjugated to one another at a cleavage site 224. Such as is shown in FIG. 2, the reporter molecule 220 can comprise a linear structure. Alternatively, the reporter molecule can comprise a secondary structure, such as a hairpin (e.g., as shown in FIG. 1). The reporter molecule 220 can comprise a moiety 222 such as a fluorophore and/or fluorescent substrate the fluorescent activity of which can be dampened, quenched, and/or otherwise halted, decreased, and/or de-activated as long as it is bound to sequence 227. The catalytic oligonucleotide 216 which can comprise a linear structure, an active conformation, and/or a predefined secondary structure that can cleave the reporter molecule at the cleavage site 224 and thereby release the moiety 222 (e.g., separate it from sequence 227) in the composition (e.g., in form of moiety 226) which can generate a detectable signal (e.g., fluorescent signal) in the composition. In some examples, this event can be referred to as the second signal amplification.

In some cases, the combination of a first signal generation and second signal generation (e.g., signal amplification) can be detected sequentially and/or simultaneously, for example, such as to generate a stronger or more intense signal, a higher signal to noise ratio, and/or other suitable signal characteristics leading to a more sensitive detection technique. For example, the first and second signal generation events can be measured at the same wavelength. Alternatively, or in addition, in some examples, the first and second signal generations can be configured to be detected at different wavelengths (e.g., with minimal to no spectral overlap). For example, the reporter molecule generating the first signal generation event can be different from the reporter molecule generating the second signal generation event. For example, more than one reporter molecule with similar or different fluorophores (e.g., similar or different detection wavelengths) can be used.

FIG. 3A shows a schematic of activation of a catalytic oligonucleotide (310) in a signal amplifier (301) comprising a catalytic oligonucleotide/blocker oligonucleotide complex by cleavage of a programmable nuclease cleavage site (314) on a blocker oligonucleotide (312) and subsequent binding of the activate catalytic oligonucleotide (317) to a reporter molecule (318) for cleavage of the reporter molecule as described herein. In some examples, the cleavage sequence 314 can comprise a segment of RNA which can be configured to be cleaved by a programmable nuclease, for example upon binding of the guide nucleic acid 115 to the target nucleic acid 116 (e.g., as shown in FIG. 1 and FIG. 2).

FIG. 3B shows a schematic of activation of a catalytic oligonucleotide (310) in a catalytic oligonucleotide/blocker oligonucleotide complex (302) by cleavage of a programmable nuclease cleavage site (314) on the blocker oligonucleotide (312), and the subsequent multi-functional capacity of the active catalytic oligonucleotide (317) to bind to a reporter molecule (318) for cleavage of the reporter molecule and/or bind to another catalytic oligonucleotide/blocker oligonucleotide complex (303) for cleavage of a catalytic oligonucleotide recognition site (316) on the blocker oligonucleotide for activation 317 of another catalytic oligonucleotide 310. In some examples, the cleavage sequence 314 can comprise a segment of RNA which can be configured to be cleaved by a programmable nuclease, for example upon binding of the guide nucleic acid 115 to the target nucleic acid 116 (e.g., as shown in FIG. 1 and FIG. 2). In this way, the catalytic oligonucleotide may be coupled to the activity of the programmable nuclease and can stimulate generation of additional signal-generating catalytic oligonucleotides in order to enhance signal generation compared to a signal generated in a system having only the programmable nuclease.

Provided herein is a composition comprising a first catalytic oligonucleotide bound to a first blocker oligonucleotide. The first blocker oligonucleotide can comprise a cleavage site and a second catalytic oligonucleotide recognition site for binding and cleaving by a second catalytic oligonucleotide. The composition can further comprise a second catalytic oligonucleotide bound to a second blocker oligonucleotide. The second blocker oligonucleotide can comprise a first catalytic recognition site for binding and cleaving by the first catalytic oligonucleotide. Upon cleavage of the cleavage site, the first catalytic oligonucleotide can bind to the first catalytic recognition site of the second blocker oligonucleotide.

In some examples, the first catalytic oligonucleotide can be configured to form a secondary structure with catalytic activity upon cleavage of the cleavage site. The first catalytic oligonucleotide can cleave the second blocker oligonucleotide so that the second catalytic oligonucleotide forms a secondary structure with catalytic activity. In some examples, the second catalytic oligonucleotide can be configured to bind to and cleave the second catalytic oligonucleotide recognition site on a first blocker oligonucleotide of another complex comprising a first catalytic oligonucleotide and a first blocker oligonucleotide, thereby releasing an additional first catalytic oligonucleotide with catalytic activity.

FIG. 4 shows a schematic of activation of a first catalytic oligonucleotide (410) in a first signal amplifier (401) comprising a first catalytic oligonucleotide/blocker oligonucleotide complex by cleavage of a programmable nuclease cleavage site (414) on the blocker oligonucleotide (412), and the subsequent multi-functional capacity of the first catalytic oligonucleotide (417) to bind to a reporter molecule (418) for cleavage of the reporter molecule and/or bind to a second catalytic oligonucleotide/blocker oligonucleotide complex (402) for cleavage of a first catalytic oligonucleotide recognition site (424) on the second blocker oligonucleotide (422) for activation of the second catalytic oligonucleotide (420). The activated second catalytic oligonucleotide (426) can subsequently bind to and cleave a second catalytic oligonucleotide recognition site (416) on another first catalytic oligonucleotide/blocker oligonucleotide complex (401) for activation 417 of another first catalytic oligonucleotide (410).

Provided herein are systems, methods, and compositions for amplifying a signal programmable nuclease detection event via the activation of one or more signal amplifiers which can initiate additional reporter cleavage events and generate more signal compared to the signal generated by the programmable nuclease alone.

### Target Nucleic Acid Detection

The methods described herein can be used to assay for or detect the presence of a target nucleic acid as disclosed herein. In some embodiments, the target nucleic acid is in a sample. In some embodiments, the target nucleic acid can comprise a nucleic acid from a pathogen. The pathogen can be associated with a disease or infection. The pathogen can be a virus, a bacterium, a protozoan, a parasite, or a fungus. The target nucleic acid can be associated with a disease trait (e.g., antibiotic resistance). In some embodiments, the target nucleic acid can comprise a variant relative to a wild type or reference genotype. In some embodiments, the target nucleic acid is a variant of a wild-type nucleic acid sequence or a variant of a reference nucleic acid sequence. The variant target nucleic acid can comprise a single nucleotide polymorphism that affects the expression of a gene. The variant can comprise multiple variant nucleotides. The variant can comprise an insertion or a deletion of one or more nucleotides. A variant can affect the expression of a gene, RNA associated with the expression of a gene, or affect regulation of expression of a gene, such as an RNA or a promoter, enhancer, or repressor of the gene. The variant can be associated with a disease phenotype, a genetic disorder, or a predisposition to a disease (e.g., cancer). Often, the detection of a variant target nucleic acid is used to diagnose or identify diseases associated with the variant target nucleic acid. The variant target nucleic acid can be detected in a population of nucleic acids comprising the wild-type nucleic acid sequence or reference nucleic acid sequence. Detection of variant nucleic acids are applicable to a number of fields, such as clinically, as a diagnostic, in laboratories as a research tool, and in agricultural applications.

The methods for detection of a target nucleic acid described herein further can comprises reagents protease treatment of the sample. The sample can be treated with protease, such as Protease K, before amplification or before assaying for a detectable signal. Often, a protease treatment is for no more than 15 minutes. Sometimes, the protease treatment is for no more than 1, 5, 10, 15, 20, 25, 30, or more minutes, or any value from 1 to 30 minutes. Sometimes, the protease treatment is from 1 to 30, from 5 to 25, from 10 to 20, or from 10 to 15 minutes.

In some examples, the methods as disclosed herein further comprise amplifying the target nucleic acid, such as by thermal amplification or isothermal amplification. Sometimes, nucleic acid amplification of the sample improves at least one of sensitivity, specificity, or accuracy of the assay in detecting the target nucleic acid. In some embodiments, nucleic acid amplification comprises amplifying using a recombinase, a oligonucleotide primer, a single-stranded DNA binding (SSB) protein, and a polymerase. Sometimes, the nucleic acid amplification is polymerase chain reaction (PCR) amplification. Sometimes, the nucleic acid amplification is isothermal nucleic acid amplification. In some cases, the nucleic acid amplification is transcription mediated amplification (TMA). Nucleic acid amplification is helicase dependent amplification (HDA) or circular helicase dependent amplification (cHDA) in other cases. In additional cases, nucleic acid amplification is strand displacement amplification (SDA). In some cases, nucleic acid amplification is by recombinase polymerase amplification (RPA). In some cases, nucleic acid amplification is by at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value from 1 to 60 minutes. Sometimes, the nucleic acid amplification is performed for from 5 to 60, from 10 to 60, from 15 to 60, from 30 to 60, from 45 to 60, from 1 to 45, from 5 to 45, from 10 to 45, from 30 to 45, from 1 to 30, from 5 to 30, from 10 to 30, from 15 to 30, from 1 to 15, from 5 to 15, or from 10 to 15 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C. In some cases, the nucleic acid amplification reaction is performed at a temperature of from 20° C. to 45° C., from 25° C. to 40° C., from 30° C. to 40° C., or from 35° C. to 40° C. In some cases, the nucleic acid amplification reaction is performed at a temperature of from 45° C. to 65° C., from 50° C. to 65° C., from 55° C. to 65° C., or from 60° C. to 65° C. In some cases, the nucleic acid amplification reaction can be performed at a temperature that ranges from about 20° C. to 45° C., from 25° C. to 45° C., from 30° C. to 45° C., from 35° C. to 45° C., from 40° C. to 45° C., from 20° C. to 37° C., from 25° C. to 37° C., from 30° C. to 37° C., from 35° C. to 37° C., from 20° C. to 30° C., from 25° C. to 30° C., from 20° C. to 25° C., or from about 22° C. to 25° C. In some cases, the nucleic acid amplification reaction can be performed at a temperature that ranges from about 40° C. to 65° C., from 45° C. to 65° C., from 50° C. to 65° C., from 55° C. to 65° C., from 60° C. to 65° C., from 40° C. to 60° C., from 45° C. to 60° C., from 50° C. to 60° C., from 55° C. to 60° C., from 40° C. to 55° C., from 45° C. to 55° C., from 50° C. to 55° C., from 40° C. to 50° C., or from about 45° C. to 50° C. In some cases, the nucleic acid amplification is performed in a nucleic acid amplification region on a support medium. Alternatively or in combination, the nucleic acid amplification is performed in a reagent chamber, and the resulting sample is applied to the support medium.

Sometimes, the total time for the performing the method described herein is no greater than 3 hours, 2 hours, 1 hour, 50 minutes, 40 minutes, 30 minutes, 20 minutes, 15 minutes, or any value from 3 hours to 10 minutes. Often, a method of nucleic acid detection from a raw sample comprises protease treating the sample for no more than 15 minutes, amplifying (can also be referred to as pre-amplifying) the sample for no more than 15 minutes, subjecting the sample to a programmable nuclease-mediated detection, and assaying nuclease mediated detection. The total time for performing this method, sometimes, is no greater than 3 hours, 2 hours, 1 hour, 50 minutes, 40 minutes, 30 minutes, 20 minutes, 15 minutes, or any value from 3 hours to 10 minutes.

### Devices

A number of detection or visualization devices and methods are consistent with the compositions and methods as disclosed herein. As described herein, a target nucleic acid can be detected using a programmable nuclease, guide nucleic acid, catalytic oligonucleotide, optionally a blocker oligonucleotide, reporter molecule, and buffers disclosed herein. In some examples, devices for carrying out the methods of detection of a target nucleic acid described herein can further comprise reagents for nucleic acid amplification of target nucleic acids in the sample, such as thermal amplification or isothermal amplification as disclosed herein. A programmable nuclease can also be multiplexed with multiple guide nucleic acids and/or multiple programmable nucleases for detection of multiple different target nucleic acids as described herein. In some embodiments, the device is any device that can measure or detect a calorimetric, potentiometric, amperometric, optical (e.g., fluorescent, colorometric, etc.), or piezo-electric signal. Often a calorimetric signal is heat produced after cleavage of the reporter molecules. Sometimes, a calorimetric signal is heat absorbed after cleavage of the reporter molecules. A potentiometric signal, for example, is electrical potential produced after cleavage of the reporter molecules. An amperometric signal can be movement of electrons produced after the cleavage of reporter molecule. Often, the signal is an optical signal, such as a colorometric signal or a fluorescence signal. An optical signal is, for example, a light output produced after the cleavage a reporter molecule. In some examples, an optical signal is a change in light absorbance between before and after the cleavage of reporter molecules. In some cases, a piezo-electric signal is a change in mass between before and after the cleavage of the reporter molecule. In some cases, the reporter molecule is a protein-nucleic acid. In some cases, the protein-nucleic acid is an enzyme-nucleic acid.

In some instances, systems or devices for detecting a target nucleic acid comprise a support medium; a guide nucleic acid targeting a target sequence; a programmable nuclease capable of being activated when complexed with the guide nucleic acid and the target sequence; a signal amplifier; and a reporter nucleic acid comprising a detection moiety, wherein the reporter nucleic acid is capable of being cleaved by the activated programmable nuclease and/or activated signal amplifier, thereby releasing the detection moiety (or releasing a quenching moiety and exposing the detection moiety) and generating a first detectable signal.

In some instances, systems for detecting a target nucleic acid are configured to perform one or more steps of the DETECTR assay in a volume or on the support medium. In some instances, one or more steps of the DETECTR assay are performed in the same volume or at the same location on the support medium. For example, target nucleic acid amplification can occur in a separate volume before the programmable nuclease complex (also referred to herein as an RNP) is contacted to the amplified target nucleic acids. In another example, target nucleic acid amplification can occur in the same volume in which the target nucleic acids complex with the RNP (e.g., amplification can occur in a sample well or tube before the RNP is added and/or amplification and RNP complexing can occur in the sample well or tube simultaneously). In another example, the DETECTR assay can occur with prior target nucleic acid amplification. Detection of the detectable signal indicative of transcollateral cleavage of the reporter nucleic acid can occur in the same volume or location on the support medium (e.g., sample well or tube after or simultaneously with transcleavage) or in a different volume or location on the support medium (e.g., at a detection location on a lateral flow assay strip, at a detection location in a well, or at a detection spot in a microarray). In some instances, all steps of the DETECTR assay can be performed in the same volume or at the same location on the support medium. For example, optional target nucleic acid amplification, complexing of the RNP with the target nucleic acid, transcollateral cleavage of the reporter nucleic acid, signal amplification by the signal amplifier, and generation of the detectable signal can occur in the same volume (e.g., sample well or tube). Alternatively, or in combination, target nucleic acid amplification, complexing of the RNP with the target nucleic acid, transcollateral cleavage of the reporter nucleic acid, signal amplification by the signal amplifier, and generation of the detectable signal can occur at the same location on the support medium (e.g., on a bead in a well or flow channel).

The results from the detection region from a completed assay can be detected and analyzed in various ways. For example, by a glucometer. In some cases, the positive control spot and the detection spot in the detection region is visible by eye, and the results can be read by the user. In some cases, the positive control spot and the detection spot in the detection region is visualized by an imaging device or other device depending on the type of signal. Often, the imaging device is a digital camera, such a digital camera on a mobile device. The mobile device can have a software program or a mobile application that can capture an image of the support medium, identify the assay being performed, detect the detection region and the detection spot, provide image properties of the detection spot, analyze the image properties of the detection spot, and provide a result. Alternatively or in combination, the imaging device can capture fluorescence, ultraviolet (UV), infrared (IR), or visible wavelength signals. The imaging device can have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease, cancer, or genetic disorder. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, clean-up of an environment.

### Kits

The compositions as disclosed herein can be provided as kits for use in detecting any number of target nucleic acids disclosed herein in a laboratory setting (e.g., as a research tool or for clinical grade testing) or direct to consumer product. A kit can comprise a target nucleic acid, a programmable nuclease, guide nucleic acid, catalytic oligonucleotide, optionally a blocker oligonucleotide, reporter molecule, and buffers disclosed herein. In some examples, a kit further comprises reagents for nucleic acid amplification of target nucleic acids in the sample, such as thermal amplification or isothermal amplification as disclosed herein. In some embodiments, a kit comprises more than one programmable nuclease, which is multiplexed for detection of multiple different target nucleic acids as described herein, and/or comprises multiple guide nucleic acids for detection of multiple different target nucleic acids. Kits can be provided as co packs for open box instrumentation.

In other embodiments, the compositions or kits as disclosed herein can be used in a point-of-care (POC) test, which can be carried out at a decentralized location such as a hospital, POL, or clinic. These point-of-care tests can be used to diagnose any of the indications disclosed herein, such as influenza or streptococcal infections, or can be used to measure the presence or absence of a particular variant in a target nucleic acid (e.g., EGFR). POC tests can be provided as small instruments with a consumable test card, wherein the test card is any of the assay formats (e.g., a lateral flow assay) disclosed herein.

In still other embodiments, compositions or kits as described herein can be used in an over-the-counter (OTC), readerless format, which can be used at remote sites or at home to diagnose a range of indications. These indications can include influenza, streptococcal infections, or CT/NG infections. OTC products can include a consumable test card, wherein the test card is any of the assay formats (e.g., a lateral flow assay) disclosed herein. In an OTC product, the test card can be interpreted visually or using a mobile phone.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Terminology used herein is for the purpose of describing particular embodiments only and is not intended to be limiting of the invention. For example, as used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present invention.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Percent identity,” “% identity,” and % “identical” refers to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some instances, the subject is not necessarily diagnosed or suspected of being at high risk for the disease.

The term “effector protein” refers to a protein that is capable of modifying a nucleic acid molecule (e.g., by cleavage, deamination, recombination). Modifying the nucleic acid may modulate the expression of the nucleic acid molecule (e.g., increasing or decreasing the expression of a nucleic acid molecule). The effector protein may be a Cas protein (i.e., an effector protein of a CRISPR-Cas system).

### EXAMPLES

The following examples are illustrative and non-limiting to the scope of the methods, systems, devices, and kits described herein.

### Example 1

### Reporter Molecule Cleavage by LbuCas13a

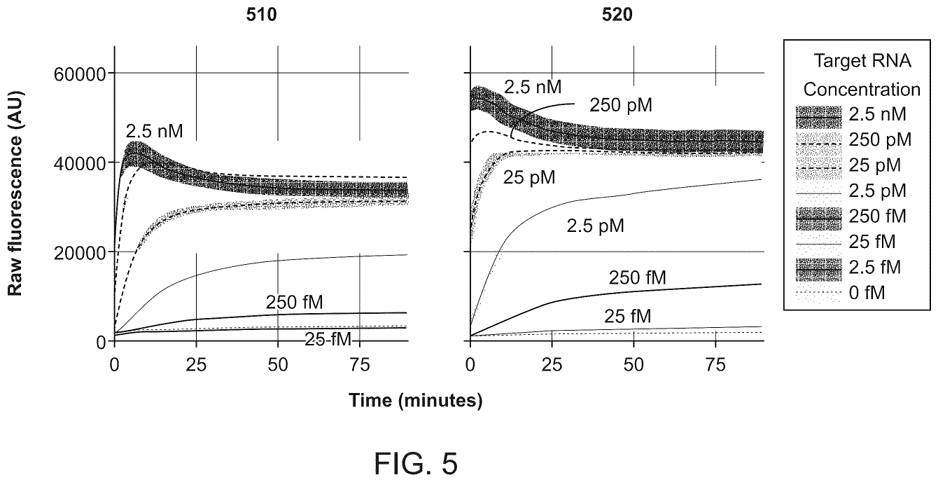

This example illustrates cleavage of two example reporter molecules by LbuCas13a (SEQ ID NO. 19) and describes that a reporter molecule which were configured to be cleaved by DNAzymes also efficiently cleaved by LbuCas13a and can be suitable for performing the methods of the present disclosure. Provided herein are such reporter molecules, DNAzymes, programmable nucleases such as LbuCas13a, and systems for performing the methods of the present disclosure. FIG. 5 shows an example experiment in which a programmable nuclease (LbuCas13a) was demonstrated to cleave two example reporter molecules (reporter molecule 510 and reporter molecule 520). The performance of the nuclease LbuCas13a in cleaving the reporter molecules were illustrated and compared. Reporter molecule 510 was a reporter molecule (DZ-beacon-1) designed for a DNAzyme. Stated a different way, reporter molecule 510 was configured to be cleaved by both a programmable nuclease such as LbuCas13a or other Cas enzymes and by a DNAzyme. Reporter molecule 520 was a reporter molecule (rep001) which was optimized for cleavage by the programmable nuclease only.

In this example, LbuCas13a complexing reaction was performed at 37° C. for about 30 minutes with 40 nanoMolar (nM) Cas protein and 40 nM CRISPR RNA (crRNA). 15 μL of LbuCas13a complexing reaction was added to 5 microLiter (μL) of target RNA with either reporter molecule 510 or reporter molecule 520 (i.e., a reporter molecule for cleavage by LbuCas13a). The reaction was allowed to proceed for about 90 minutes at about 37° C. The target nucleic acid in this example was R440, and the CRISPR RNA (crRNA) was R015, the sequences of which are provided below in TABLE 10 below.

TABLE 10
Target Nucleic Acid Sequences and cRNA Nucleic Acid Sequences
Used in Experiments

| | Sequence (SEQ ID NO) |
|---|---|
| Target | CGCUGAUGGUACUAUAUACAAGAGUAUGGGAGAGUAGGUCGUCGCCA |
| Nucleic | AGC (SEQ ID NO: 206) |
| Acid | |
| R440 | |
| cRNA | GGCCACCCCAAAAAUGAAGGGGACUAAAACACGACCUACUCUCCCAUA |
| R015 | CUC (SEQ ID NO: 207) |

Results from a dilution series of target RNA, for example at concentrations of 0 femtomolar (fM), 2.5 fM, 25 fM, 250 fM, 2.5 picomolar (pM), 25 pM, 250 pM, and 2.5 nanomolar (nM) as shown on the graphs, indicated that in this example, the programmable nuclease was capable of cleaving and/or activating both reporter molecules, with about the same or similar efficiency. In some instances, reporter molecule 510 which is configured to be cleaved by Cas enzymes as well as DNAzymes can be used to perform the methods of the present disclosure, such as the methods generally described in FIGS. 1-2, FIGS. 3A-3B, and FIG. 4.

### Example 2

### Reporter Molecule Cleavage by LbuCas13a in Two Example Buffers

This example shows the effect of buffer on reporter molecule cleavage by LbuCas13a. Two example buffers (CutSmart and MBuffer1) were used in the experiments provided in this example, and fluorescence signals generated over time were measured. The results reported in this example provided information about example buffers which can be used in the methods and systems of the present disclosure and the effects thereof on reporter molecule cleavage by LbuCas13a which can be considered in choice of buffer.

In this example, a set of experiments were performed to study the effects of assay conditions, such as assay buffers (e.g., buffer chemistry and reagents) and concentration of reagents such as MgCl₂ in example buffers (e.g., CutSmart buffer and MBuffer1) which can be used in the methods and systems of the present disclosure on the performance of an example programmable nuclease (LbuCas13a) and an example DNAzyme (DZ-act-linear). For example, the results of these experiments can be used to identify a buffer to be used in the methods of the present disclosure, such as to reach a suitable performance level for LbuCas13a.

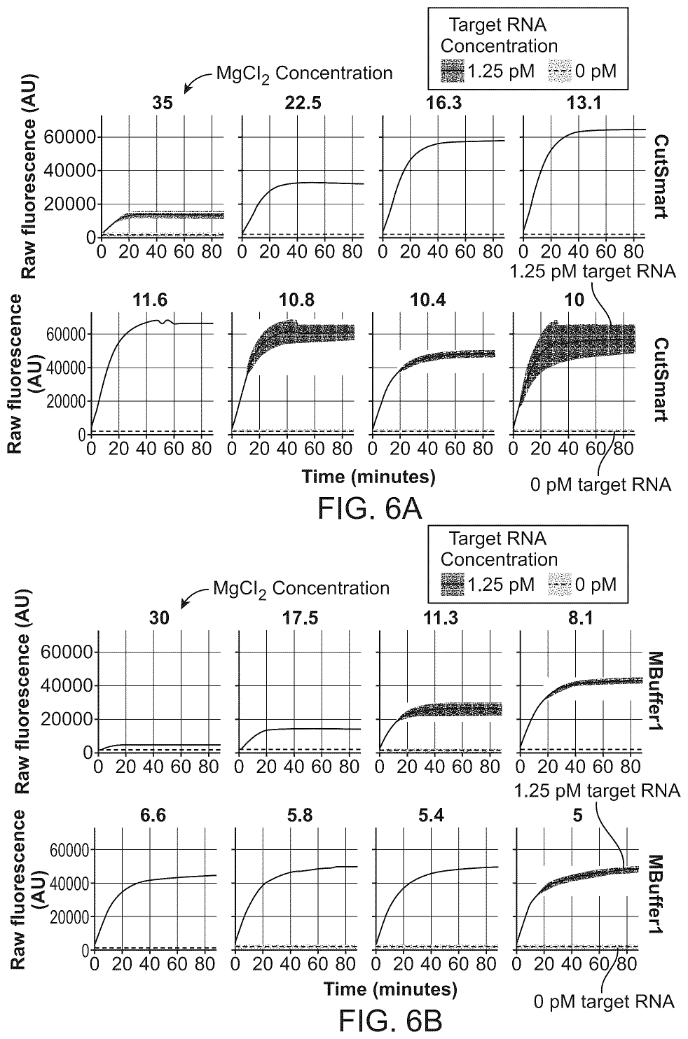

FIG. 6A and FIG. 6B show the results of a set of experiments which were performed to study the effect of buffer and components thereof on an example programmable nuclease (LbuCas13a). A complexing reaction (complexing the programmable nuclease LbuCas13a with guide nucleic acid) was performed at 37° C. for 30 minutes with 40 nanoMolar (nM) protein (e.g., Cas13) and 40 nM CRISPR RNA (crRNA). 10 μL of the LbuCas13a complexing reaction solution was added to 5 μL of buffers with varying MgCl₂ dilutions. 1.25 pM final concentration of target RNA or 0 pM target RNA (control sample) was added to the reaction. The reaction was allowed to proceed for about 90 minutes at about 37° C. Fluorescent signals generated from cleavage of reporters by LbuCas13a were measured over time and are shown in the plots of FIGS. 6A and 6B. It should be understood that these conditions are provided as examples, and various alternative experimental conditions, buffers, and incubation conditions can be used.

FIG. 6A shows fluorescent signals over time in a sample comprising CutSmart buffer with varying concentrations of MgCl₂. An example recipe for a 1× CutSmart buffer can comprise about 50 millimolar (mM) Potassium acetate, about 20 mM Tris-acetate, about 10 mM Magnesium acetate, about 100 microgram per milliliter (μg/ml) BSA, and a PH of about 7.9 at 25° C. In some cases, the CutSmart buffer can be purchased as a 10× buffer and can be diluted as needed. In some cases, the PH range of the 10× CutSmart buffer can be from about 7.8 to about 8.0. The PH of the buffer can be adjusted to any suitable value depending on the experiment. In some examples, the buffer used in the experiments of the present disclosure can comprise CutSmart buffer and Magnesium chloride (MgCl₂) at varying concentrations. The concentration of MgCl₂ can be adjusted to optimize the performance of the assays and/or the activity of the components of the compositions.

In the example shown in FIG. 6A, the MgCl₂ concentrations tested in the CutSmart buffer were 35 milliMolar (mM), 22.5 mM, 16.3 mM, 13.1 mM, 11.6 mM, 10.8 mM, 10.4 mM, and 10 mM. The results obtained for each condition (e.g., each MgCl₂ concentration) are shown in a separate plot in FIG. 6A, illustrating the effect of the concentration of MgCl₂ on the performance of the programmable nuclease (LbuCas13a). Performance of a programmable nuclease in an assay can comprise or be assessed by various factors, such as the intensity of the measured fluorescent signal (e.g., at a given time point), the rate of signal amplification (e.g., the rate of increase in the fluorescent signal over time), the signal to noise ratio, the average, standard deviation, coefficient of variability, regression value of the signal, combinations thereof, and more. A similar experiment was performed to test the performance of programmable nuclease (LbuCas13a) in another example buffer (MBuffer1), the results of which are provided in FIG. 6B.

FIG. 6B shows the measured fluorescent signals over time in a sample comprising MBuffer1 with varying concentrations of MgCl₂. An example recipe for MBuffer1 can comprise 100 mM Imidazole pH 7.5; 250 mM KCl, 25 mM MgCl₂, 50 ug/mL BSA, 0.05% Igepal Ca-630, and 25% Glycerol. The MgCl₂ concentrations tested in MBuffer1 were 30 milliMolar (mM), 17.5 mM, 11.3 mM, 8.1 mM, 6.6 mM, 5.8 mM, 5.4 mM, and 5 mM, the results of which are provided in separate plots in FIG. 6B. The top curve in each plot indicates a LbuCas13a concentration of 1.25 picoMolar (pM). The bottom curve in each plot indicates a LbuCas13a concentration of 0 (pM).

Various other buffers and reagents at various concentrations can be used to perform the methods of the present disclosure. In some examples, compositions can comprise MgCl₂ at concentrations of equal to or greater than about 20 mM. In some examples, compositions can comprise MgCl₂ at concentrations at least about 1 mM, 2 mM, 3 mM, 4 mM, 4 mM, 5 mM, 6 mM, 7 mM, 8 mM, 9 mM, 10 mM, 12 mM, 15 mM, 16 mM, 20 mM or more. In some examples, assay conditions, such as buffers and concentrations of reagents can need to be adjusted such as to optimize the performance of the programmable nuclease and/or the performance of DNAzymes, for example to reach a suitable performance level for both, and/or an overall optimized condition for both. For example, an optimal condition can comprise a buffer chemistry and concentration at which the combined performance of the DNAzyme and the programmable nuclease can be optimized, leading to a proper overall outcome for the assay.

From the experimental data shown in FIGS. 6A and 6B, it was observed that in this particular example, among the tested variations, the performance of programmable nuclease (LbuCas13a) was best while using the CutSmart buffer in MgCl₂ concentrations below 16 mM. Therefore, this particular example shows that in some instances, having an MgCl₂ concentration of at most about 16 mM in a buffer (e.g., CutSmart buffer) used in the methods of the present disclosure can be optimal for performance of LbuCas13a.

### Example 3

### Reporter Molecule Cleavage by DNAzyme in Two Example Buffers

This example shows the effect of buffer on reporter molecule cleavage by a DNAzyme (an activatable oligonucleotide which can be used in the methods of the present disclosure). Two example buffers (CutSmart and MBuffer1) were used in the experiments provided in this example, and fluorescence signals generated over time were measured. The results reported in this example can provide information about example buffers which can be used in the methods and systems of the present disclosure and the effects thereof on reporter cleavage by DNAzymes which can be considered in choice of buffer.

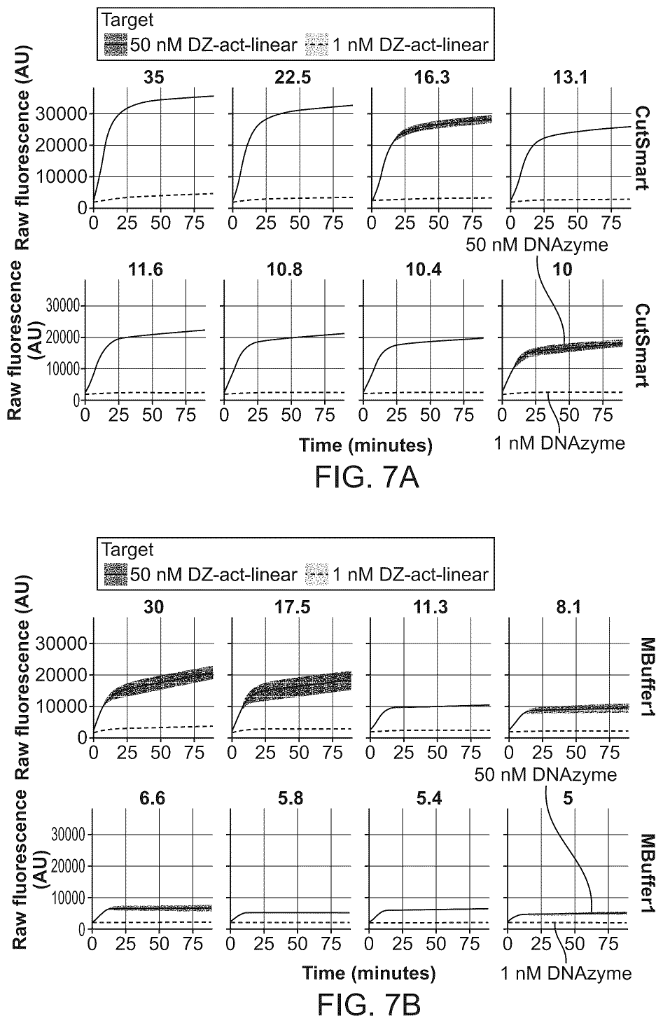

FIGS. 7A and 7B show experimental data on the performance of an example DNAzymes in two example buffers (CutSmart buffer and MBuffer1 buffer). In these experiments, 10 μL aliquots of the DNAzyme (DZ-beacon-1) at 100 nM reaction concentration were prepared in reaction buffer with murine RNase inhibitor (NEB). 5 μL of MgCl₂ serial dilutions were added to the aliquots. 5 μL of DNAzyme (DZ-act-linear) was added at a final concentration of either 50 nM or 1 nM, immediately before the start of measuring the fluorescent output of the reaction. The reaction was allowed to proceed for 90 minutes at 37° C. The performance of the DNAzyme was evaluated in both buffers (CutSmart in FIG. 7A and MBuffer1 in FIG. 7B) at varying concentrations of MgCl₂ in each buffer. The plots shown in FIG. 7A show the results of the experiments at MgCl₂ concentrations of about 35 mM, 22.5 mM, 16.3 mM, 13.1 mM, 11.6 mM, 10.8 mM, 10.4 mM, and 10 mM in CutSmart buffer. FIG. 7B shows representative plots illustrating the results of varying MgCl₂ concentrations on reporter cleavage by the tested DNAzyme. Tested MgCl₂ concentration about 30 mM, 17.5 mM, 11.3 mM, 8.1 mM, 6.6 mM, 5.8 mM, 5.4 mM, and 5 mM in MBuffer1. The top curve in each graph indicates a DNAzyme (DZ-act-linear) concentration of about 50 nM. The bottom curve in each curve indicates a DNAzyme (DZ-act-linear) concentration of about 1 nM. It was observed that in this particular example, the performance of the DNAzyme decreased below 16 mM MgCl₂ in the CutSmart buffer. Therefore, the results of this particular example show that in some instances, including MgCl₂ at a concentration of at least about 16 nM in a buffer (e.g., CutSmart buffer) used to perform the methods of the present disclosure can be optimal for the performance of DNAzymes.

### Example 4

### Activating DNAzymes Through Cleavage of Blocker Oligonucleotides by Cas Enzymes

This example illustrates inactivation of a DNAzyme using a blocker oligonucleotide and further re-activating the DNAzyme through the cleavage of the blocker oligonucleotide by a programmable nuclease, such as LbuCas13a. As explained in further detail elsewhere herein, blocker oligonucleotides can force DNAzyme into structures other than their active structure, thereby yielding an inactive DNAzyme. For example, blocker oligonucleotides can force a DNAzyme into a substantially circular structure which does not allow DNAzyme to reach its target and perform its activity. A Cas enzyme can cleave the blocker oligonucleotide and facilitate the return of the DNAzyme to its active structure, thereby activating the DNAzyme. Examples of methods comprising activating an inactive DNAzyme by nuclease-mediated cleavage are provided generally in FIGS. 1-2, FIGS. 3A-3B, and FIG. 4. The example described in this section also presents example concentrations of the blocker oligonucleotides which can be used for inactivating DNAzymes. Further, in this example, examples optimal concentration ranges for the blocker oligonucleotide, DNAzymes, and their relative ration (blocker oligonucleotide: DNAzyme ratio) are reported.

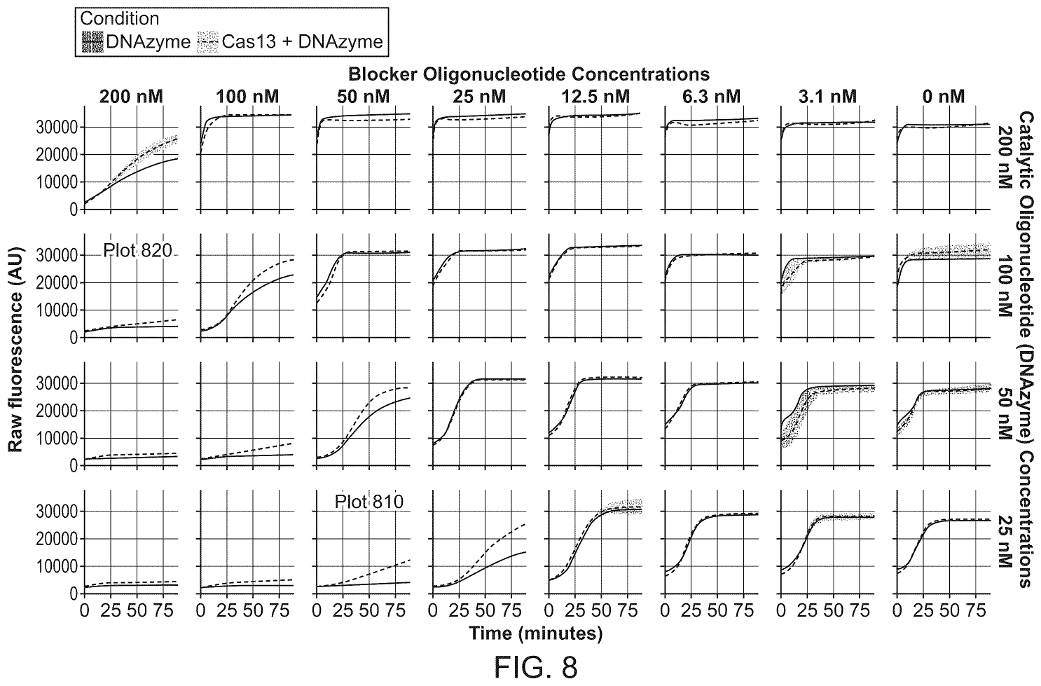

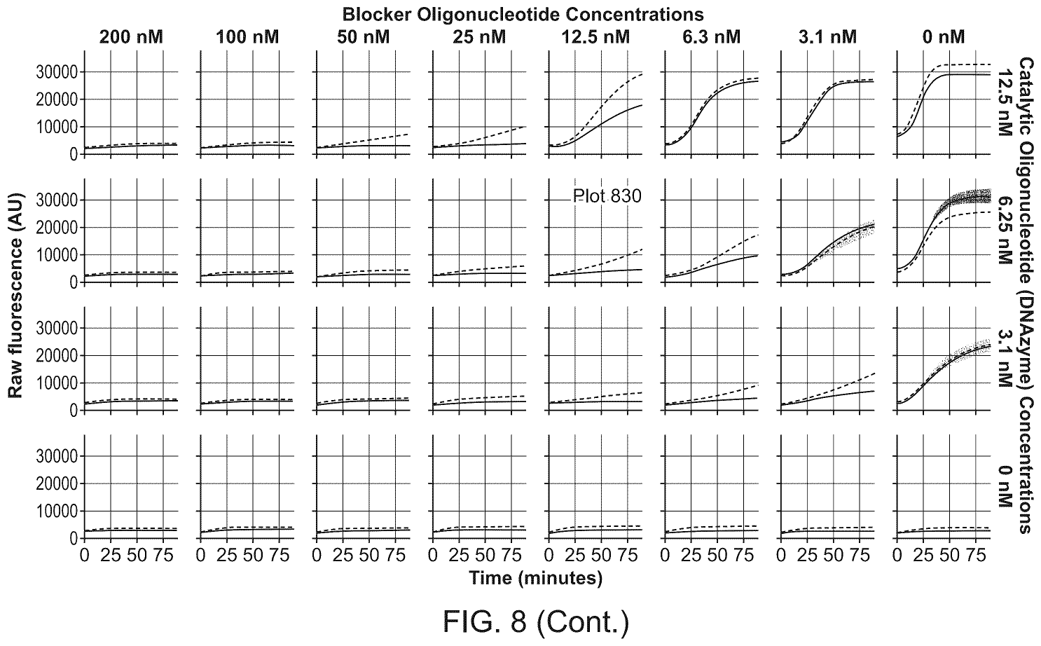

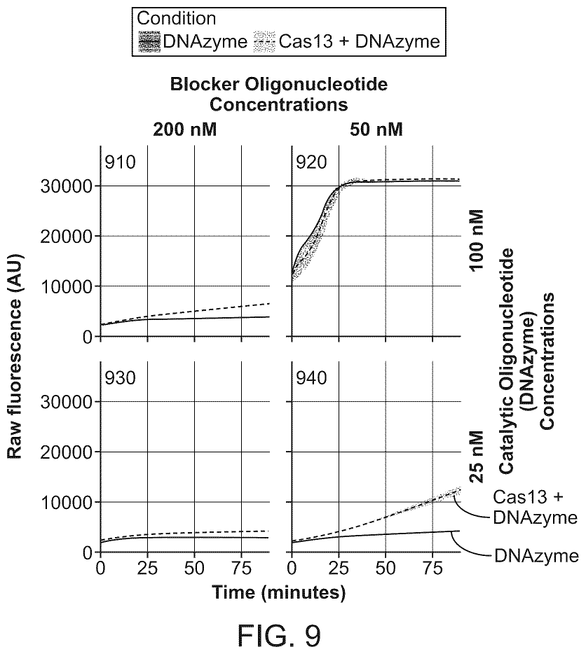

FIG. 8 and FIG. 9 show the results of a set of experiments that were performed to determine an optimal concentration of an example blocker oligonucleotide and/or its ratio relative to an example DNAzyme and an example programmable nuclease (LbuCas13a) in a composition provided herein. Various concentrations of the blocker oligonucleotide, the DNAzyme, and the programmable nuclease (LbuCas13a) were tested.

The results of these experiments are provided in the plots shown in FIG. 8. In these experiments, various dilutions of the DNAzyme (e.g., DZ-act-linear) and blocker oligonucleotide were prepared and allowed to anneal in 1× CutSmart buffer at a final MgCl₂ concentration of 20 nM. Concentrations of the blocker oligonucleotides are provided above each column of the plots shown in FIG. 8. The concentrations of the DNAzyme are provided on the right side of the rows in FIG. 8. Experiments were performed in presence and absence of programmable nuclease (LbuCas13a). LbuCas13a complexing reactions with 40 nM gRNA and 40 nM protein (e.g., Cas protein) were prepared and added to the DNAzyme-blocker dilutions. Reporter and RNA target were prepared and added to the annealed DNAzyme-blocker+LbuCas13a mixes. Reactions were allowed to proceed for 90 minutes at 37° C. The results indicated that in this particular example, an optimal performance of the assay was reached while using a blocker oligonucleotide to DNAzyme ratio of about 2:1. In an optimal condition, the ratio of the blocker oligonucleotide to DNAzyme was about 2:1, concentration of blocker oligonucleotide was about 50 nM, and concentration of DNAzyme was about 25 nM (see plot 810). In another optimal condition, the concentration of blocker oligonucleotide was about 12.5 nM and concentration of DNAzyme was about 6.3 nM (see plot 830).

In another example, the ratio of blocker oligonucleotide to DNAzyme was about 2:1, the blocker oligonucleotide concentration was about 200 nM and the DNAzyme concentration was about 100 nM (See plot 820). In this condition, there is an excess 100 nM of blocker oligonucleotide that can need to be cleaved by the programmable nuclease (LbuCas13a) in order to fully release the DNAzyme. In some cases, it may not be preferred to have significant excess of blocker oligonucleotide. Therefore, the concentration of blocker oligonucleotide can be decreased.

In other examples, various concentrations of the blocker oligonucleotide, DNAzyme, and programmable nucleases at various relative ratios can work. See, generally plots shown in FIG. 8 as examples. In some cases, optimal conditions can comprise a 2:1 ratio of blocker oligonucleotides to DNAzymes at blocker oligonucleotide concentrations of less than about 100 nM, such as 90 nM, 80 nM, 70 nM, 60 nM, 50 nM, 40 nM, 30 nM, 20 nM, 13 nM, or less. The DNAzyme concentration in each case can be about half of (50%) of the concentration of the blocker oligonucleotide.

FIG. 9 shows the performance of a method and composition/system provided herein at various concentrations of the DNAzyme and the blocker oligonucleotide in presence and absence of the programmable nuclease (Cas13). The data provided in the plots of FIG. 9 show fluorescent signals (AU) over time (minutes) in the mentioned conditions. The columns indicate the concentration of the blocker oligonucleotide, while the rows indicate the concentration of the DNAzyme. In each plot, two curves are illustrated in presence and absence of Cas13. The fluorescent signals were obtained at least partially due to the cleavage of the reporter molecule by the activated DNAzymes and/or by the programmable nuclease (LbuCas13a).

With continued reference to FIG. 9, Plot 910 is representative an experiment in which the composition comprised 200 nM blocker oligonucleotide and a 100 nM DNAzyme in presence (top curve) and absence (bottom curve) of Cas13. The results indicate the inhibition of DNAzyme by the blocker oligonucleotide. lower signals compared to other conditions (e.g., conditions shown in the rest of the plots) were measured from the Cas13-mediated cleavage of the reporter molecule under these assay conditions.

Plot 920 shows the results of an experiment in which the composition comprised 50 nM blocker oligonucleotide and 100 nM DNAzyme. The experiment was performed in presence and absence of Cas13. No significant difference was observable between the two curves. The results indicate little to no inhibition of DNAzyme (e.g., by the blocker oligonucleotide) was observed under these assay conditions. A strong signal was observed in absence of LbuCas13a (e.g., compared to the curve measured in presence of same).

Plot 930 shows the results of an experiment in which the composition or system comprised 200 nM blocker oligonucleotide and 25 nM DNAzyme in presence and absence of LbuCas13a. Minimal to no difference among the two curves was observed. The results indicate inhibition of DNAzyme and weakest performance with Cas13M36 coupling.

Plot 940 shows the results of an experiment in which the composition comprised 50 nM blocker oligonucleotide and 25 nM DNAzyme. The top curve was obtained in presence of Cas13. The bottom curve was obtained in absence of Cas13. The results indicate inhibition of DNAzymes by the blocker oligonucleotides. The strongest LbuCas13a signals was observed in plot 940 compared to the other plots. Therefore, the conditions used in plot 940 can be preferred compared to the other ones. In other examples, the conditions can be further adjusted and/or optimized to achieve suitable results.

### Example 5

### Reporter Molecule Cleavage by a Composition Comprising a Programmable Nuclease and a DNAzyme

This example illustrates the cleavage of a reporter molecule (rep091) by a programmable nuclease (LbuCas13a) and a DNAzyme (M1634 Dz2) using the methods of the present disclosure, such as the methods and systems generally described elsewhere herein, such as in FIGS. 1-2, FIGS. 3A-3B, and FIG. 4. In the example described in this section, the reporter molecule is rep091. The nuclease sequence (LbuCas13a) is provided in Table 1. The sequence of the DNAzyme (M1634 Dz2) is provided in Table 7. The sequence of the blocker oligonucleotide (Dz2-blocker-U5) is provided in Table 7. In this example, the sequence of the crRNA was R015, the sequence of which is provided in Table 10, and target nucleic acid was R440, the sequence of which is provided in Table 10.

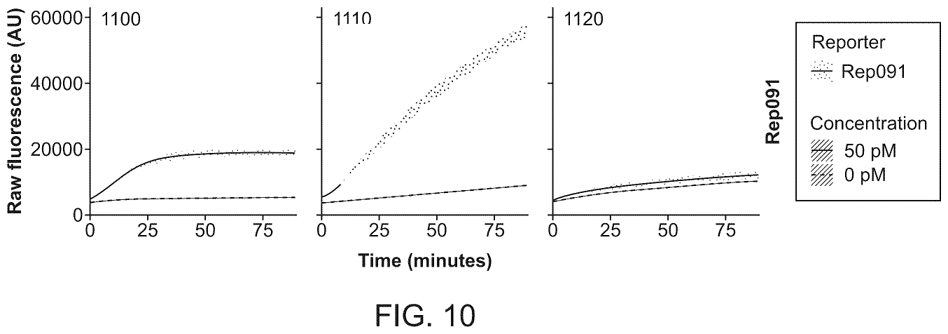

FIG. 10 shows the results of a set of experiments in which the combined effects of Cas13 coupled with a DNAzyme were tested and compared to conditions in which either the Cas13 or the DNAzyme was absent. In these experiments, 25 nM DNAzyme and 12.5 nM rU5-blocker oligonucleotide were annealed at room temperature for about 30 minutes. LbuCas13a complexing reaction was performed at 37° C. for 30 minutes with 40 nM protein and 40 nM crRNA. 5 μL of LbuCas13a complexing reaction was added to 10 μL of annealed DNAZyme and blocker oligonucleotides. 5 μL of DNAzyme reporter (rep091) and 50 pM target nucleic acid molecule (top curve in each plot) or no-target RNA were added, and the reaction was allowed to proceed for 90 minutes at 37° C. In these experiments, the Cas13 used was LbuCas13a, the DNAzyme was M1634 Dz2, the blocker oligonucleotide was Dz2-blocker-U5, the reporter molecule was rep091, crRNA was R015, and target nucleic acid was R440. The results of the experiments are shown in FIG. 10.

Plot 1100 shows the results of incubating Cas13 in absence of DNAzyme with the target nucleic acid molecule at concentrations of 50 pM (top cuve) and 0 pM (bottom curve). Plot 1110 shows the results of incubating both Cas13 and the DNAzyme with the target nucleic acid molecule at concentrations of 50 pM (top curve) and 0 pM (bottom curve). Plot 1120 shows the results of incubating DNAzyme in absence of Cas13 with the target nucleic acid molecule at molecule at concentrations of 50 pM (top curve) and 0 pM (bottom curve). Fluorescent signals generated in each case were measured over time and presented in the plots. Results indicated that in this particular example, when Cas13a was coupled to the DNAzyme system, the reaction demonstrated different kinetics, and the signal after 90 minutes at 37° C. was found to be higher than that of Cas13a in absence of DNAzymes.

While various embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A composition comprising a signal amplifier, a programmable nuclease, and a guide nucleic acid that hybridizes to a segment of a target nucleic acid.

2. The composition of claim 1, wherein the signal amplifier comprises an enzyme.

3. The composition of claim 1, wherein the signal amplifier comprises a catalytic oligonucleotide.

4. The composition of claim 3, wherein the catalytic oligonucleotide has a circular structure.

5. The composition of claim 3, wherein the catalytic oligonucleotide comprises a programmable nuclease cleavage site.

6. (canceled)

7. The composition of claim 3, further comprising a blocker oligonucleotide.

8. (canceled)

9. (canceled)

10. The composition of claim 7, wherein the blocker oligonucleotide comprises a programmable nuclease cleavage site, a catalytic oligonucleotide recognition site, or a combination thereof.

11. (canceled)

12. The composition of claim 3, wherein the catalytic oligonucleotide comprises an enzyme.

13. The composition of claim 3, wherein the catalytic oligonucleotide comprises a DNAzyme or a ribozyme.

14.-17. (canceled)

18. The composition of claim 1, wherein the programmable nuclease is a type VI CRISPR/Cas effector protein or a type V CRISPR/Cas effector protein.

19.-27. (canceled)

28. The composition of claim 1, further comprising the target nucleic acid.

29. The composition of claim 28, wherein the target nucleic acid is a target RNA or a target DNA.

30. (canceled)

31. The composition of claim 1, further comprising a reporter molecule.

32. The composition of claim 31, wherein the reporter molecule is configured to generate a signal upon cleavage by a catalytic oligonucleotide, the programmable nuclease, or both.

33. (canceled)

34. (canceled)

35. The composition of claim 1, wherein the programmable nuclease is a first programmable nuclease and the composition further comprises a second programmable nuclease.

36. A composition comprising a first signal amplifier, a second signal amplifier, a programmable nuclease, and a guide nucleic acid that hybridizes to a segment of a target nucleic acid.

37. The composition of claim 36, wherein the first signal amplifier comprises a first enzyme and the second signal amplifier comprises a second enzyme.

38. The composition of claim 36, wherein the first signal amplifier comprises a first catalytic oligonucleotide and the second signal amplifier comprises a second catalytic oligonucleotide.

39.-72. (canceled)

73. A method of nucleic acid detection comprising:
(a) contacting a sample to a composition comprising a plurality of reporter molecules and the composition of claim 1; and
(b) assaying for a signal produced by or indicative of cleavage one or more of the reporter molecules.

74.-80. (canceled)

81. A method of nucleic acid detection comprising:
(a) contacting a sample comprising a plurality of nucleic acids to a composition comprising a plurality of reporter molecules, a programmable nuclease complex comprising a programmable nuclease coupled to a guide nucleic acid that hybridizes to a segment of a target nucleic acid, and a signal amplifier;
(b) when the target nucleic acid is present in the plurality of nucleic acids, activating the programmable nuclease complex by hybridizing the target nucleic acid, or an amplicon thereof, to the guide nucleic acid;
(c) activating the signal amplifier with the activated programmable nuclease complex, wherein the activated signal amplifier is configured to cleave at least a reporter molecule of the plurality of reporter molecules; and
(d) assaying for a signal produced by or indicative of cleavage of the reporter molecule.
